(19) United States
(12) Patent Application Publication
(10) Pub. No.: US 2025/0145585 A1
(43) Pub. Date: May 8, 2025

(54) HETEROCYCLIC INHIBITORS OF IGF-1R FOR TREATMENT OF DISEASE

(71) Applicant: HORIZON THERAPEUTICS IRELAND DAC, DUBLIN 2 (IE)

(72) Inventors: ASHLEY FENWICK, NAPERVILLE, IL (US); MARTA E. WENZLER, CHICAGO, IL (US); DENIS BILLEN, BARRINGTON, IL (US); YEEMAN K. RAMTOHUL, MONTREAL (CA); WILLIAM BROWN, MONTREAL (CA); NARESH VEMULA, DORION (CA); FRANCIS BARABE, MONTREAL (CA); THOMAS PINTER, MONTREAL (CA); NICHOLAS WILLET MITCHELL MICHEL, Montreal (CA); THOMAS JOHNSON, SAINT-LAURENT (CA); JACOB SOLEY, SAINT LAURENT (CA); GEOFFREY S. SINCLAIR, MONTREAL (CA); KEN LOON CHOO, MONTREAL (CA)

(21) Appl. No.: 18/777,460

(22) Filed: Jul. 18, 2024

Related U.S. Application Data
(60) Provisional application No. 63/514,302, filed on Jul. 18, 2023.

(51) Int. Cl. C07D 401/06; A61K 9/00; A61K 9/08; A61K 9/20; A61K 9/48; A61K 31/437; A61K 31/4375; A61K 31/4439; A61K 31/444; A61K 31/4545; A61K 31/501; A61K 31/506; A61K 31/5377; A61K 31/5383; A61K 31/675; A61K 31/695; C07D 401/14; C07D 403/06; C07D 405/14; C07D 409/14; C07D 417/14; C07D 471/04; C07D 491/048; C07D 493/08; C07D 498/04; C07D 513/04; C07F 7/08; C07F 9/58

(52) U.S. Cl. CPC C07D 401/06; A61K 9/0053; A61K 9/08; A61K 9/2013; A61K 9/2054; A61K 9/4816; A61K 9/4825; A61K 9/4866; A61K 31/437; A61K 31/4375; A61K 31/4439; A61K 31/444; A61K 31/4545; A61K 31/501; A61K 31/506; A61K 31/5377; A61K 31/5383; A61K 31/675; A61K 31/695; C07D 401/14; C07D 403/06; C07D 405/14; C07D 409/14; C07D 417/14; C07D 471/04; C07D 491/048; C07D 493/08; C07D 498/04; C07D 513/04; C07F 7/0812; C07F 9/58

(57) ABSTRACT

The present invention relates to heterocyclic compounds, pharmaceutical compositions thereof, and methods which may be useful as inhibitors of IGF-1R for the treatment or prevention of thyroid eye disease (TED), also known as thyroid-associated ophthalmopathy (TAO), or Graves' ophthalmopathy or orbitopathy (GO).

### CROSS-REFERENCE TO RELATED APPLICATIONS

This application claims the benefit of priority to U.S. Provisional Patent Application No. 63/514,302, filed Jul. 18, 2023, which is hereby incorporated by reference in its entirety as if fully set forth herein.

### BACKGROUND OF THE INVENTION

Disclosed herein are novel heterocyclic compounds and compositions and their use in the treatment of diseases or disorders, including but not limited to thyroid eye disease (TED). Methods of inhibition of insulin-like growth factor 1 receptor (IGF-1R) activity in a mammal are also provided for the treatment diseases and disorders such as thyroid eye disease (TED), also known as thyroid-associated ophthalmopathy (TAO), or Graves' ophthalmopathy or orbitopathy (GO).

TED is typically associated with Graves' hyperthyroidism but can also occur as part of other autoimmune conditions that affect the thyroid gland and produce pathology in orbital and periorbital tissue, and, rarely, the pretibial skin (pretibial myxedema) or digits (thyroid acropachy). TED is an autoimmune orbitopathy in which the orbital and periocular soft tissues are primarily affected with secondary effects on the eye and vision. In TED, as a result of inflammation and expansion of orbital soft tissues, primarily eye muscles and adipose, the eyes are forced forward (bulge) out of their sockets—a phenomenon termed proptosis or exophthalmos.

TED is commonly considered to be the autoimmune orbital manifestation of Graves' Disease (GD). However, only approximately 30% of patients with Graves' hyperthyroidism manifest clinically relevant ocular pathology indicating there is mechanistic heterogeneity and differentiation between the conditions. Although the molecular mechanisms underlying TED remain unclear, it is accepted that the generation of autoantibodies that act as agonists on the thyroid-stimulating hormone receptor (TSHR) is responsible for Graves' hyperthyroidism. Pathogenic overstimulation of TSHR, leads to overproduction of thyroid hormones (T3 and T4) and accelerated metabolism of many tissues.

Antibodies that activate the insulin-like growth factor 1 receptor (IGF-1R) have also been detected and implicated in active TED. Without being bound to any theory, it is believed that TSHR and IGF-1R form a physical and functional complex in orbital fibroblasts, and that blocking IGF-1R appears to attenuate both IGF-1 and TSH-dependent signaling. It has been suggested that blocking IGF-1R activity might reduce both TSHR- and IGF-1-dependent signaling and therefore interrupt the pathological activities of autoantibodies acting as agonists on either receptor.

IGF-1R is a widely expressed heterotetrameric protein involved in the regulation of proliferation and metabolic function of many cell types. It is a tyrosine kinase receptor comprising two subunits. IGF-1Rα contains a ligand-binding domain while IGF-1Rβ is involved in signaling and contains tyrosine phosphorylation sites.

Management of hyperthyroidism due to Graves' disease is imperfect because therapies targeting the specific underlying pathogenic autoimmune mechanisms of the disease are lacking. Even more complex is the treatment of moderate-to-severe active TED. Although recent years have witnessed a better understanding of its pathogenesis, TED remains a therapeutic challenge and dilemma. Intravenous glucocorticoids (ivGCs) and oral glucocorticoids are used to treat patients with moderate-to-severe active TED, but results are seldom satisfactory. Partial responses are frequent and relapses (rebound) after drug withdrawal are not uncommon. Adverse events do occur and many patients eventually require rehabilitative surgery conducted when their condition has transitioned to inactive TED. Teprotumumab, a fully human IGF-1R inhibitory monoclonal antibody, is the only therapeutic specifically approved for patients with active, moderate-to-severe TED.

There is a need for novel therapeutics, specifically small molecules, that inhibit IGF-1R activity.

### BRIEF SUMMARY OF THE INVENTION

Compounds disclosed herein inhibit IGF-1R activity.

In one aspect, described herein is a compound of Formula (I):

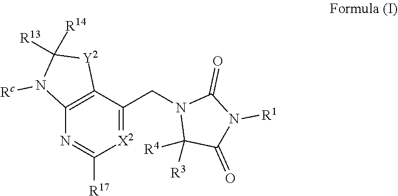

or a pharmaceutically acceptable salt or solvate thereof, wherein:
R¹ is unsubstituted or substituted phenyl, unsubstituted or substituted monocyclic heteroaryl, or unsubstituted or substituted bicyclic heteroaryl; wherein if R¹ is substituted, then it is substituted with R^{A} and 1-3 R⁵;
   R^{A} is unsubstituted or substituted C₁-C₆ alkyl, unsubstituted or substituted C₁-C₆ fluoroalkyl, unsubstituted or substituted phenyl, unsubstituted or substituted 5- or 6-membered heteroaryl, unsubstituted or substituted C₃-C₆ cycloalkyl, unsubstituted or substituted C₃-C₆ cycloalkenyl, unsubstituted or substituted 3- to 6-membered heterocycloalkyl, halogen, —CN, —SR^{A1}, —S(═O)R^{A1}, —S(═O)₂R^{A1}, —SF₅, or —SiMe₃;
      R^{A1} is C₁-C₆ alkyl, C₁-C₆ fluoroalkyl, C₃-C₆ cycloalkyl, or 3- to 6-membered heterocycloalkyl;
   or R^{A} is

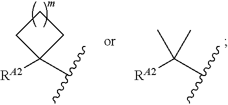

R² is halogen, —OH, —CN, C₁-C₄ alkyl, C₁-C₄ fluoroalkyl, C₁-C₄ alkoxy, C₁-C₄ fluoroalkoxyl, or C₁-C₄ hydroxyalkyl;
m is 0, 1, 2, or 3;
each R⁵ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²¹, —CO₂R²², —N(R²²)₂, —C(═O)N(R²²)₂, —NR²²C(═O)R²¹, —S(═O)₂N(R²²)₂, or —NR²²S(═O)₂R²¹;
or R¹ is

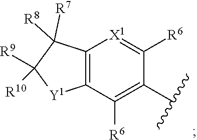

X¹ is CR⁶ or;
   each R⁶ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²¹, —CO₂R²², —N(R²²)₂, —C(═O)N(R²²)₂, —NR²²C(═O)R²¹, —S(═O)₂N(R²², or —NR²²S(═O)₂R²¹;
Y¹ is —O—, —NR¹¹—, or —SO₂—;
   R¹¹ is hydrogen, C₁-C₆ alkyl, C₁-C₆ fluoroalkyl, —C(═O)NH₂, —C(═O)R¹², or —S(═O)₂R¹²;
   R¹² is unsubstituted or substituted C₁-C₆ alkyl, unsubstituted or substituted C₃-C₆ cycloalkyl, or unsubstituted or substituted 3- to 6-membered heterocycloalkyl;
R⁷ and R⁸ are each independently hydrogen, —OH, C₁-C₆ alkyl, C₁-C₆ fluoroalkyl, or C₁-C₆ alkoxy;
or R⁷ and R⁸ are taken together with the carbon atom to which they are attached to form an unsubstituted or substituted C₃-C₆ cycloalkyl or unsubstituted or substituted 3- to 6-membered heterocycloalkyl;
R⁹ and R¹⁰ are each independently hydrogen, halogen, C₁-C₆ alkyl, C₁-C₆ fluoroalkyl, or C₁-C₆ alkoxy;
or R⁹ and R¹⁰ are taken together with the carbon atom to which they are attached to form a —C(═O)—;
or R⁹ and R¹⁰ are taken together with the carbon atom to which they are attached to form an unsubstituted or substituted C₃-C₆ cycloalkyl or unsubstituted or substituted 3- to 6-membered heterocycloalkyl;
R³ is hydrogen, unsubstituted or substituted C₁-C₆ alkyl, unsubstituted or substituted C₁-C₆ fluoroalkyl, unsubstituted or substituted C₁-C₆ alkoxy, unsubstituted or substituted phenyl, unsubstituted or substituted benzyl, unsubstituted or substituted monocyclic heteroaryl, unsubstituted or substituted C₃-C₆ cycloalkyl, or unsubstituted or substituted 3- to 6-membered heterocycloalkyl;
R⁴ is hydrogen or C₁-C₆ alkyl;
or R³ and R⁴ are taken together with the carbon atom to which they are attached to form a C₃-C₆ cycloalkyl or 3- to 6-membered heterocycloalkyl;
R^{c} is hydrogen, C₁-C₆ alkyl, C₁-C₆ fluoroalkyl, —C(═O)N(R²²)₂, —C(═O)R²¹, —C(═O)OR²², or —S(═O)₂R²¹;
X² is CR¹⁷ or N;
each R¹⁷ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²¹, —CO₂R²², —N(R²²)₂, —C(═O)N(R²²)₂, —OC(═O)R²¹, —NR²²C(═O)R²¹, —S(═O)₂N(R²²)₂, or —NR²²S(═O)₂R²¹;
R¹³ and R¹⁴ are each independently hydrogen, halogen, unsubstituted or substituted C₁-C₆ alkyl, C₁-C₆ fluoroalkyl, phenyl, monocyclic heteroaryl, C₃-C₆ cycloalkyl, 3- to 6-membered heterocycloalkyl, —CN, —OH, —OR²¹, —CO₂R²², —N(R²²)₂, —C(═O)N(R²²)₂, or —OC(═O)R²¹;
or R¹³ and R¹⁴ are taken together with the carbon atom to which they are attached to form a —C(═O)—;
or R¹³ and R¹⁴ are taken together with the carbon atom to which they are attached to form a C₃-C₆ cycloalkyl or 3- to 6-membered heterocycloalkyl;
Y² is —(CR¹⁵R¹⁶)ₙ—, O—, or —O—CR¹⁵R¹⁶—;
   each R¹⁵ and R¹⁶ is independently hydrogen, halogen, cyano, C₁-C₆ alkyl, C₁-C₆ fluoroalkyl, C₁-C₆ alkoxy, phenyl, monocyclic heteroaryl, C₃-C₆ cycloalkyl, or 3- to 6-membered heterocycloalkyl;
   or R¹⁴ and one R¹⁶ on an adjacent carbon atom are taken together to form an alkene bond;
   n is 1 or 2;
each R²¹ is independently unsubstituted or substituted C₁-C₆ alkyl, unsubstituted or substituted C₃-C₆ cycloalkyl, unsubstituted or substituted 3- to 6-membered heterocycloalkyl, unsubstituted or substituted aryl, or unsubstituted or substituted heteroaryl;
each R²² is independently hydrogen, unsubstituted or substituted C₁-C₆ alkyl, unsubstituted or substituted C₃-C₆ cycloalkyl, unsubstituted or substituted 3- to 6-membered heterocycloalkyl, unsubstituted or substituted aryl, or unsubstituted or substituted heteroaryl; or two R²² on the same N atom are taken together with the N atom to which they are attached to form an unsubstituted or substituted N-containing heterocycloalkyl;
wherein each substituted alkyl, substituted fluoroalkyl, substituted aryl, substituted heteroaryl, substituted cycloalkyl, and substituted heterocycloalkyl is substituted with one or more R⁵ groups independently selected from the group consisting of halogen, C₁-C₆ alkyl, C₁-C₆ fluoroalkyl, C₃-C₆ cycloalkyl, 3- to 6-membered heterocycloalkyl, phenyl, benzyl, 5-membered heteroaryl, 6-membered heteroaryl. —CN, —OR²³, —CO₂R²³, —C(═O)N(R²³)₂, —N(R²³)₂, —NR²³C(═O)R²⁴, —SR²³, —S(═O)R²⁴, —SO₂R²⁴, or —SO₂N(R²³)₂;
each R²³ is independently selected from hydrogen, C₁-C₆ alkyl, C₃-C₆ cycloalkyl, 3- to 6-membered heterocycloalkyl, phenyl, benzyl, 5-membered heteroaryl and 6-membered heteroaryl; or two R²³ groups are taken together with the N atom to which they are attached to form a 3- to 6-membered N-containing heterocycloalkyl;
each R²⁴ is independently selected from C₁-C₆ alkyl, C₃-C₆ cycloalkyl, 3- to 6-membered heterocycloalkyl, phenyl, benzyl, 5-membered heteroaryl and 6-membered heteroaryl.

In another aspect described herein is a compound of Formula (II):

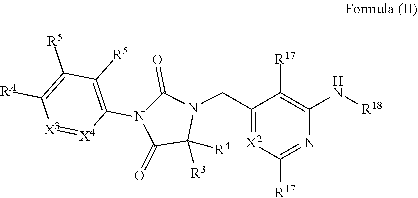

or a pharmaceutically acceptable salt or solvate thereof, wherein:
R^{A} is unsubstituted or substituted C₁-C₆ fluoroalkyl, unsubstituted or substituted phenyl, unsubstituted or substituted 5- or 6-membered heteroaryl, unsubstituted or substituted C₃-C₆ cycloalkyl, unsubstituted or substituted C₃-C₆ cycloalkenyl, or unsubstituted or substituted 3- to 6-membered heterocycloalkyl, halogen, —CN, —SF₅, or —SiMe₃;
or R^{A} is

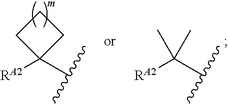

R^{A2} is halogen, —OH, —CN, C₁-C₄ alkyl, C₁-C₄ fluoroalkyl, C₁-C₄ alkoxy, C₁-C₄ fluoroalkoxyl, or C₁-C₄ hydroxyalkyl;
m is 0, 1, 2, or 3;
X³ and X⁴ are each independently CR⁵ or N;
R³ is hydrogen, unsubstituted or substituted C₁-C₆ alkyl, unsubstituted or substituted C₁-C₆ fluoroalkyl, unsubstituted or substituted C₁-C₆ alkoxy, unsubstituted or substituted phenyl, unsubstituted or substituted benzyl, unsubstituted or substituted monocyclic heteroaryl, unsubstituted or substituted C₃-C₆ cycloalkyl, or unsubstituted or substituted 3- to 6-membered heterocycloalkyl;
R⁴ is hydrogen or C₁-C₆ alkyl;
or R³ and R⁴ are taken together with the carbon atom to which they are attached to form a C₃-C₆ cycloalkyl or 3- to 6-membered heterocycloalkyl;
each R⁵ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²¹, —CO₂R²², —N(R²²)₂, —C(═O)N(R²²)₂, —NR²²C(═O)R²¹, —S(═O)₂N(R²²)₂, or —NR²²S(═O)₂R²¹;
X² is CR¹⁷ or N;
each R¹⁷ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²¹, —CO₂R²². —N(R²²)₂, —C(═O)N(R²²)₂, —NR²²C(═O)R²¹, —S(═O)₂N(R²²)₂, or —NR²²S(═O)₂R²¹;
R¹⁸ is hydrogen, unsubstituted or substituted C₁-C₆ alkyl, unsubstituted or substituted C₁-C₆ fluoroalkyl, unsubstituted or substituted C₁-C₆ heteroalkyl, unsubstituted or substituted C₁-C₆ alkenyl, unsubstituted or substituted C₁-C₆ alkynyl, unsubstituted or substituted carbocycle, unsubstituted or substituted heterocycle, —CHR²⁰-(unsubstituted or substituted carbocycle), —CHR²¹-(unsubstituted or substituted heterocycle), —C(═O)R¹⁹, —CO₂R¹⁹, or —C(═O)NHR¹⁹;
   R¹⁹ is hydrogen, unsubstituted or substituted C₁-C₆ alkyl, unsubstituted or substituted C₃-C₁₀ cycloalkyl, unsubstituted or substituted 3- to 10-membered heterocycloalkyl;
   R²⁰ is hydrogen or —CH₃;
each R²¹ is independently unsubstituted or substituted C₁-C₆ alkyl, unsubstituted or substituted C₃-C₆ cycloalkyl, unsubstituted or substituted 3- to 6-membered heterocycloalkyl, unsubstituted or substituted aryl, or unsubstituted or substituted heteroaryl;
each R²² is independently hydrogen, unsubstituted or substituted C₁-C₆ alkyl, unsubstituted or substituted C₃-C₆ cycloalkyl, unsubstituted or substituted 3- to 6-membered heterocycloalkyl, unsubstituted or substituted aryl, or unsubstituted or substituted heteroaryl, or two R²² on the same N atom are taken together with the N atom to which they are attached to form an unsubstituted or substituted N-containing heterocycloalkyl;
wherein each substituted alkyl, substituted fluoroalkyl, substituted aryl, substituted heteroaryl, substituted cycloalkyl, and substituted heterocycloalkyl is substituted with one or more R^{s} groups independently selected from the group consisting of halogen, C₁-C₆ alkyl, C₁-C₆ fluoroalkyl, C₃-C₆ cycloalkyl, 3- to 6-membered heterocycloalkyl, phenyl, benzyl, 5-membered heteroaryl, 6-membered heteroaryl, —CN, —OR²³, —CO₂²³, —C(═O)N(R²³)₂, —N(R²³)₂, —NR²³C(═O)R²⁴, —SR²¹, —S(═O)R²⁴, —SO₂R²⁴, or —SO₂N(R²³)₂;
   each R²³ is independently selected from hydrogen, C₁-C₆ alkyl, C₃-C₆ cycloalkyl, 3- to 6-membered heterocycloalkyl, phenyl, benzyl, 5-membered heteroaryl and 6-membered heteroaryl; or two R²³ groups are taken together with the N atom to which they are attached to form a 3- to 6-membered N-containing heterocycloalkyl;
each R²⁴ is independently selected from C₁-C₆ alkyl, C₃-C₆ cycloalkyl, 3- to 6-membered heterocycloalkyl, phenyl, benzyl, 5-membered heteroaryl and 6-membered heteroaryl.

In another aspect described herein is a compound of Formula (III):

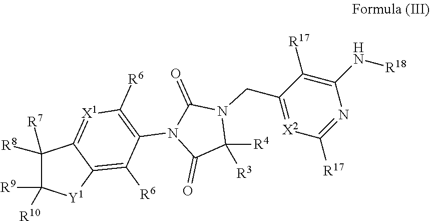

or a pharmaceutically acceptable salt or solvate thereof, wherein:
   X¹ is CR⁶ or N;
      each R⁶ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²¹, —CO₂R²², —N(R¹)₂, —C(═O)N(R²²)₂, —NR²²C(═O)R²¹, —S(═O)₂N(R²²)₂, or —NR²²S(═O)₂R²¹;
   Y¹ is —O—, —NR¹¹—, or —SO₂—;
      R¹¹ is hydrogen, C₁-C₆ alkyl, C₁-C₆ fluoroalkyl, —C(═O)NH₂, —C(═O)R¹², or —S(═O)₂R¹²,
   R¹² is unsubstituted or substituted C₁-C₆ alkyl, unsubstituted or substituted C₃-C₆ cycloalkyl, or unsubstituted or substituted 3- to 6-membered heterocycloalkyl;
   R⁷ and R⁸ are each independently hydrogen, —OH, C₁-C₆ alkyl, C₁-C₆ fluoroalkyl, or C₁-C₆ alkoxy;
   or R⁷ and R⁸ are taken together with the carbon atom to which they are attached to form an unsubstituted or substituted C₃-C₆ cycloalkyl or unsubstituted or substituted 3- to 6-membered heterocycloalkyl;
   R⁹ and R¹⁰ are each independently hydrogen, halogen, C₁-C₆ alkyl, C₁-C₆ fluoroalkyl, or C₁-C₆ alkoxy;
   or R⁹ and R¹⁰ are taken together with the carbon atom to which they are attached to form a —C(═O)—;
   or R⁹ and R¹⁰ are taken together with the carbon atom to which they are attached to form an unsubstituted or substituted C₃-C₆ cycloalkyl or unsubstituted or substituted 3- to 6-membered heterocycloalkyl;
R³ is hydrogen, unsubstituted or substituted C₁-C₆ alkyl, unsubstituted or substituted C₁-C₆ fluoroalkyl, unsubstituted or substituted C₁-C₆ alkoxy, unsubstituted or substituted phenyl, unsubstituted or substituted benzyl, unsubstituted or substituted monocyclic heteroaryl, unsubstituted or substituted C₃-C₆ cycloalkyl, or unsubstituted or substituted 3- to 6-membered heterocycloalkyl;
R⁴ is hydrogen or C₁-C₆ alkyl;
or R³ and R⁴ are taken together with the carbon atom to which they are attached to form a C₃-C₆ cycloalkyl or 3- to 6-membered heterocycloalkyl;
X² is CR¹⁷ or N;
each R¹⁷ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²¹, —CO₂R²², —N(R²²)₂, —C(═O)N(R²²)₂, —NR²²C(═O)R²¹, —S(═O)₂N(R²²)₂, or —NR²²S(═O)₂R²¹;
R¹⁸ is unsubstituted or substituted C₁-C₆ alkyl, unsubstituted or substituted C₁-C₆ fluoroalkyl, unsubstituted or substituted C₁-C₆ heteroalkyl, unsubstituted or substituted C₁-C₆ alkenyl, unsubstituted or substituted C₁-C₆ alkynyl, unsubstituted or substituted C₃-C₁₀ cycloalkyl, unsubstituted or substituted 3- to 10-membered heterocycloalkyl, —CHR²⁰-(unsubstituted or substituted carbocycle), —CHR²⁰-(unsubstituted or substituted heterocycle), —C(═O)R¹⁹, —CO₂R¹⁹, or —C(═O)NHR¹⁹;
   R¹⁹ is hydrogen, unsubstituted or substituted C₁-C₆ alkyl, unsubstituted or substituted C₃-C₁₀ cycloalkyl, or unsubstituted or substituted 3- to 10-membered heterocycloalkyl;
   R²⁰ is hydrogen or —CH₃;
each R²¹ is independently unsubstituted or substituted C₁-C₆ alkyl, unsubstituted or substituted C₃-C₆ cycloalkyl, unsubstituted or substituted 3- to 6-membered heterocycloalkyl, unsubstituted or substituted aryl, or unsubstituted or substituted heteroaryl;
each R²² is independently hydrogen, unsubstituted or substituted C₁-C₆ alkyl, unsubstituted or substituted C₃-C₆ cycloalkyl, unsubstituted or substituted 3- to 6-membered heterocycloalkyl, unsubstituted or substituted aryl, or unsubstituted or substituted heteroaryl; or two R²² on the same N atom are taken together with the N atom to which they are attached to form an unsubstituted or substituted N-containing heterocycloalkyl;
wherein each substituted alkyl, substituted fluoroalkyl, substituted aryl, substituted heteroaryl, substituted cycloalkyl, and substituted heterocycloalkyl is substituted with one or more R^{s} groups independently selected from the group consisting of halogen, C₁-C₆ alkyl, C₁-C₆ fluoroalkyl, C₃-C₆ cycloalkyl, 3- to 6-membered heterocycloalkyl, phenyl, benzyl, 5-membered heteroaryl, 6-membered heteroaryl, —CN, —OR²³, —CO₂R²³, —C(═O)N(R²³)₂, —N(R²³)₂, —NR²³C(═O)R²⁴, —SR²³, —S(═O)R²⁴, —SO₂R²⁴, or —SO₂N(R²³)₂;
   each R²³ is independently selected from hydrogen, C₁-C₆ alkyl, C₃-C₆ cycloalkyl, 3- to 6-membered heterocycloalkyl, phenyl, benzyl, 5-membered heteroaryl and 6-membered heteroaryl; or two R²³ groups are taken together with the N atom to which they are attached to form a 3- to 6-membered N-containing heterocycloalkyl;
   each R²⁴ is independently selected from C₁-C₆ alkyl, C₃-C₆ cycloalkyl, 3- to 6-membered heterocycloalkyl, phenyl, benzyl, 5-membered heteroaryl and 6-membered heteroaryl.

In another aspect described herein is a compound of Formula (IV):

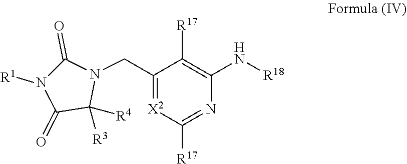

or a pharmaceutically acceptable salt or solvate thereof, wherein:
R¹ is unsubstituted or substituted bicyclic heteroaryl; wherein if R¹ is substituted, then it is substituted with R^{A} and 1-3 R⁵;
   R^{A} is unsubstituted or substituted C₁-C₆ alkyl, unsubstituted or substituted C₁-C₆ fluoroalkyl, unsubstituted or substituted C₃-C₆ cycloalkyl, unsubstituted or substituted 3- to 6-membered heterocycloalkyl, halogen, —CN, —SR^{A1}, —S(═O)R^{A1}, —S(═O)₂R^{A1}, —OR^{A1}, —SF₅, or —SiMe₃;
      R^{A1} is C₁-C₆ alkyl, C₁-C₆ fluoroalkyl, C₃-C₆ cycloalkyl, or 3- to 6-membered heterocycloalkyl:
   each R⁵ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²¹, —CO₂R²², —N(R²²)₂, —C(═O)N(R²²)₂, —NR²²C(═O)R²¹, —S(═O)₂N(R²²)₂, or —NR²²S(═O)₂R²¹;
R³ is hydrogen, unsubstituted or substituted C₁-C₆ alkyl, unsubstituted or substituted C₁-C₆ fluoroalkyl, unsubstituted or substituted C₁-C₆ alkoxy, unsubstituted or substituted phenyl, unsubstituted or substituted benzyl, unsubstituted or substituted monocyclic heteroaryl, unsubstituted or substituted C₃-C₆ cycloalkyl, or unsubstituted or substituted 3- to 6-membered heterocycloalkyl;
R⁴ is hydrogen or C₁-C₆ alkyl;
or R³ and R⁴ are taken together with the carbon atom to which they are attached to form a C₃-C₆ cycloalkyl or 3- to 6-membered heterocycloalkyl;
X² is CR¹⁷ or N;
each R¹⁷ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²¹, —CO₂R²², —N(R²²)₂, —C(═O)N(R²²)₂, —NR²²C(═O)R²¹, —S(═O)₂N(R²²)₂, or —NR²²S(═O)₂R²¹;
R¹⁸ is hydrogen, unsubstituted or substituted C₁-C₆ alkyl, unsubstituted or substituted C₁-C₆ fluoroalkyl, unsubstituted or substituted C₁-C₆ heteroalkyl, unsubstituted or substituted C₁-C₆ alkenyl, unsubstituted or substituted C₁-C₆ alkynyl, unsubstituted or substituted carbocycle, unsubstituted or substituted heterocycle, —CHR²⁰-(unsubstituted or substituted carbocycle), —CHR²⁰-(unsubstituted or substituted heterocycle), —C(═O)R¹⁹, —CO₂R¹⁹, or —C(═O)NHR¹⁹;
   R¹⁹ is hydrogen, unsubstituted or substituted C₁-C₆ alkyl, unsubstituted or substituted C₃-C₁₀ cycloalkyl, or unsubstituted or substituted 3- to 10-membered heterocycloalkyl;
   R²⁰ is hydrogen or —CH₃;
each R²¹ is independently unsubstituted or substituted C₁-C₆ alkyl, unsubstituted or substituted C₃-C₆ cycloalkyl, unsubstituted or substituted 3- to 6-membered heterocycloalkyl, unsubstituted or substituted aryl, or unsubstituted or substituted heteroaryl;
each R²² is independently hydrogen, unsubstituted or substituted C₁-C₆ alkyl, unsubstituted or substituted C₃-C₆ cycloalkyl, unsubstituted or substituted 3- to 6-membered heterocycloalkyl, unsubstituted or substituted aryl, or unsubstituted or substituted heteroaryl; or two R²² on the same N atom are taken together with the N atom to which they are attached to form an unsubstituted or substituted N-containing heterocycloalkyl;
wherein each substituted alkyl, substituted fluoroalkyl, substituted aryl, substituted heteroaryl, substituted cycloalkyl, and substituted heterocycloalkyl is substituted with one or more R^{s} groups independently selected from the group consisting of halogen, C₁-C₆ alkyl, C₁-C₆ fluoroalkyl, C₃-C₆ cycloalkyl, 3- to 6-membered heterocycloalkyl, phenyl, benzyl, 5-membered heteroaryl, 6-membered heteroaryl, —CN, —OR²³, —CO₂R²³, —C(═O)N(R²³)₂, —N(R²³)₂, —NR²³C(═O)R²⁴, —SR²³, —S(═O)R²⁴, —SO₂R²⁴, or —SO₂N(R²³)₂;
   each R²³ is independently selected from hydrogen, C₁-C₆ alkyl, C₃-C₆ cycloalkyl, 3- to 6-membered heterocycloalkyl, phenyl, benzyl, 5-membered heteroaryl and 6-membered heteroaryl; or two R²³ groups are taken together with the N atom to which they are attached to form a 3- to 6-membered N-containing heterocycloalkyl;
   each R²⁴ is independently selected from C₁-C₆ alkyl, C₃-C₆ cycloalkyl, 3- to 6-membered heterocycloalkyl, phenyl, benzyl, 5-membered heteroaryl and 6-membered heteroaryl.

In another aspect described herein is a compound of Formula (V):

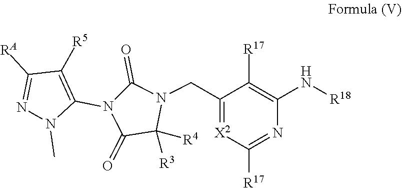

or a pharmaceutically acceptable salt or solvate thereof, wherein:
R^{A} is unsubstituted or substituted C₁-C₆ alkyl, unsubstituted or substituted C₁-C₆ fluoroalkyl, unsubstituted or substituted C₃-C₆ cycloalkyl, unsubstituted or substituted 3- to 6-membered heterocycloalkyl, halogen, —CN, —SR^{A1}, —S(═O)R^{A1}, —S(═O)₂R^{A1}, —OR^{A1}, —SF₅, or —SiMe₃;
   R^{A1} is C₁-C₆ alkyl, C₁-C₆ fluoroalkyl, C₃-C₆ cycloalkyl, or 3- to 6-membered heterocycloalkyl;
R³ is hydrogen, unsubstituted or substituted C₁-C₆ alkyl, unsubstituted or substituted C₁-C₆ fluoroalkyl, unsubstituted or substituted C₁-C₆ alkoxy, unsubstituted or substituted phenyl, unsubstituted or substituted benzyl, unsubstituted or substituted monocyclic heteroaryl, unsubstituted or substituted C₃-C₆ cycloalkyl, or unsubstituted or substituted 3- to 6-membered heterocycloalkyl;
R⁴ is hydrogen or C₁-C₆ alkyl;
or R³ and R⁴ are taken together with the carbon atom to which they are attached to form a C₃-C₆ cycloalkyl or 3- to 6-membered heterocycloalkyl;
R⁵ is hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²¹, —CO₂R²², —N(R²²)₂, —C(═O)N(R²²)₂, —NR═C(═O)R²¹, —S(═O)₂N(R²²)₂, or —NR²²S(═O)₂R²¹;
X² is CR¹⁷ or N;
each R¹⁷ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²¹, —CO₂R²², —N(R²²)₂, —C(═O)N(R²)₂, —NR═C(═O)R²¹, —S(═O)₂N(R²²)₂, or —NR²²S(═O)₂R²¹;
R¹⁸ is hydrogen, unsubstituted or substituted C₁-C₆ alkyl, unsubstituted or substituted C₁-C₆ fluoroalkyl, unsubstituted or substituted C₁-C₆ heteroalkyl, unsubstituted or substituted C₁-C₆ alkenyl, unsubstituted or substituted C₁-C₆ alkynyl, unsubstituted or substituted carbocycle, unsubstituted or substituted heterocycle, —CHR²⁰-(unsubstituted or substituted carbocycle), —CHR²⁰-(unsubstituted or substituted heterocycle), —C(═O)R¹⁹, —CO₂R¹⁹, or —C(═O)NHR¹⁹;
   R¹⁹ is hydrogen, unsubstituted or substituted C₁-C₆ alkyl, unsubstituted or substituted C₃-C₁₀ cycloalkyl, or unsubstituted or substituted 3- to 10-membered heterocycloalkyl;
   R²⁰ is hydrogen or —CH₃;
each R²¹ is independently unsubstituted or substituted C₁-C₆ alkyl, unsubstituted or substituted C₃-C₆ cycloalkyl, unsubstituted or substituted 3- to 6-membered heterocycloalkyl, unsubstituted or substituted aryl, or unsubstituted or substituted heteroaryl;
each R²² is independently hydrogen, unsubstituted or substituted C₁-C₆ alkyl, unsubstituted or substituted C₃-C₆ cycloalkyl, unsubstituted or substituted 3- to 6-membered heterocycloalkyl, unsubstituted or substituted aryl, or unsubstituted or substituted heteroaryl; or two R²² on the same N atom are taken together with the N atom to which they are attached to form an unsubstituted or substituted N-containing heterocycloalkyl;
wherein each substituted alkyl, substituted fluoroalkyl, substituted aryl, substituted heteroaryl, substituted cycloalkyl, and substituted heterocycloalkyl is substituted with one or more R groups independently selected from the group consisting of halogen, C₁-C₆ alkyl, C₁-C₆ fluoroalkyl, C₃-C₆ cycloalkyl, 3- to 6-membered heterocycloalkyl, phenyl, benzyl, 5-membered heteroaryl, 6-membered heteroaryl, —CN, —OR²³, —CO₂R²³, —C(═O)N(R²³)₂, —N(R²³)₂, —NR²³C(═O)R²⁴, —SR²³, —S(═O)R²⁴, —SO₂R²⁴, or —SO₂N(R²³)₂;
   each R²³ is independently selected from hydrogen, C₁-C₆ alkyl, C₃-C₆ cycloalkyl, 3- to 6-membered heterocycloalkyl, phenyl, benzyl, 5-membered heteroaryl and 6-membered heteroaryl; or two R² groups are taken together with the N atom to which they are attached to form a 3- to 6-membered N-containing heterocycloalkyl;
   each R²⁴ is independently selected from C₁-C₆ alkyl, C₃-C₆ cycloalkyl, 3- to 6-membered heterocycloalkyl, phenyl, benzyl, 5-membered heteroaryl and 6-membered heteroaryl.

In another aspect described herein is a compound of Formula (VI):

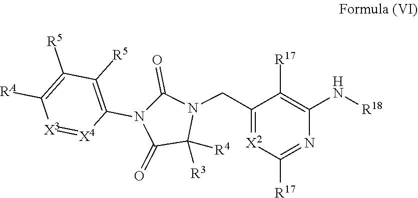

or a pharmaceutically acceptable salt or solvate thereof, wherein:
R^{A} is unsubstituted or substituted C₁-C₆ alkyl;
X³ and X⁴ are each independently CR⁵ or N,
R³ is hydrogen, unsubstituted or substituted C₁-C₆ alkyl, unsubstituted or substituted C₁-C₆ fluoroalkyl, unsubstituted or substituted C₁-C₆ alkoxy, unsubstituted or substituted phenyl, unsubstituted or substituted benzyl, unsubstituted or substituted monocyclic heteroaryl, unsubstituted or substituted C₃-C₆ cycloalkyl, or unsubstituted or substituted 3- to 6-membered heterocycloalkyl;
R⁴ is hydrogen or C₁-C₆ alkyl;
or R³ and R⁴ are taken together with the carbon atom to which they are attached to form a C₃-C₆ cycloalkyl or 3- to 6-membered heterocycloalkyl;
each R⁵ is independently hydrogen, halogen, unsubstituted or substituted C₁-C₆ alkyl, unsubstituted or substituted C₁-C₆ fluoroalkyl, —CN, —OH, —OR²¹, —CO₂R²², —N(R²²)₂, —C(═O)N(R²²)₂, —NR²²C(═O)R²¹, —S(═O)₂N(R²²)₂, or —NR²²S(═O)₂R²¹;
X² is CR¹⁷ or N;
each R¹⁷ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²¹, —CO₂R²², —N(R²²)₂, —C(═O)N(R²²)₂, —NR²²C(═O)R²¹, —S(═O)₂N(R²²)₂, or —NR²²S(═)₂R²¹;
R¹⁸ is unsubstituted or substituted C₁-C₆ alkyl, unsubstituted or substituted C₁-C₆ fluoroalkyl, unsubstituted or substituted C₁-C₆ heteroalkyl, unsubstituted or substituted C₁-C₆ alkenyl, or unsubstituted or substituted C₁-C₆ alkynyl;
or R¹⁸ is substituted cyclopropyl, unsubstituted or substituted cyclobutyl, unsubstituted or substituted cyclopentyl, or unsubstituted or substituted cyclohexyl;
or R¹⁸ is unsubstituted or substituted C₃-C₁₀ bicyclic cycloalkyl;
or R¹⁸ is unsubstituted or substituted 3- to 10-membered monocyclic or bicyclic heterocycloalkyl containing an oxygen atom;
or R¹⁸ is —CHR²-(unsubstituted or substituted carbocycle) or —CHR²⁰-(unsubstituted or substituted heterocycle);
R²⁰ is hydrogen or —CH₃;
each R²¹ is independently unsubstituted or substituted C₁-C₆ alkyl, unsubstituted or substituted C₃-C₆ cycloalkyl, unsubstituted or substituted 3- to 6-membered heterocycloalkyl, unsubstituted or substituted aryl, or unsubstituted or substituted heteroaryl;
each R²² is independently hydrogen, unsubstituted or substituted C₁-C₆ alkyl, unsubstituted or substituted C₃-C₆ cycloalkyl, unsubstituted or substituted 3- to 6-membered heterocycloalkyl, unsubstituted or substituted aryl, or unsubstituted or substituted heteroaryl; or two R²² on the same N atom are taken together with the N atom to which they are attached to form an unsubstituted or substituted N-containing heterocycloalkyl;
wherein each substituted alkyl, substituted fluoroalkyl, substituted aryl, substituted heteroaryl, substituted cycloalkyl, and substituted heterocycloalkyl is substituted with one or more R^{s} groups independently selected from the group consisting of halogen, C₁-C₆ alkyl, C₁-C₆ fluoroalkyl, C₃-C₆ cycloalkyl, 3- to 6-membered heterocycloalkyl, phenyl, benzyl, 5-membered heteroaryl, 6-membered heteroaryl, —CN, —OR²³, —CO₂R²³, —C(═O)N(R²³)₂, —N(R²³)₂, —NR²³C(═O)R²⁴, —SR²³, —S(═O)R²⁴, —SO₂R²⁴, or —SO₂N(R²³)₂;
   each R²³ is independently selected from hydrogen, C₁-C₆ alkyl, C₃-C₆ cycloalkyl, 3- to 6-membered heterocycloalkyl, phenyl, benzyl, 5-membered heteroaryl and 6-membered heteroaryl; or two R²³ groups are taken together with the N atom to which they are attached to form a 3- to 6-membered N-containing heterocycloalkyl;
   each R²⁴ is independently selected from C₁-C₆ alkyl, C₃-C₆ cycloalkyl, 3- to 6-membered heterocycloalkyl, phenyl, benzyl, 5-membered heteroaryl and 6-membered heteroaryl.

In another aspect described herein is a compound of Formula (VII):

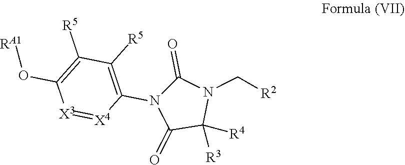

or a pharmaceutically acceptable salt or solvate thereof, wherein:
R^{A1} is C₁-C₆ alkyl, C₁-C₆ fluoroalkyl, C₃-C₆ cycloalkyl, or 3- to 6-membered heterocycloalkyl;
X³ and X⁴ are each independently CR⁵ or N;
R² is

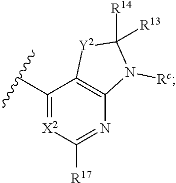

R^{c} is hydrogen, C₁-C₆ alkyl, C₁-C₆ fluoroalkyl, —C(═O)N(R²²)₂, —C(═O)R²¹, —C(═O)OR²², or —S(═O)₂R²¹;
X² is CR¹⁷ or N;
each R¹⁷ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²¹, —CO₂R²², —N(R²²)₂, —C(═O)N(R²²)₂, —OC(═O)R²¹, —NR²²C(═O)R²¹, —S(═O)₂N(R²²)₂, or —NR²²S(═O)₂R²¹;
R¹³ and R¹⁴ are each independently hydrogen, halogen, unsubstituted or substituted C₁-C₆ alkyl, C₁-C₆ fluoroalkyl, phenyl, monocyclic heteroaryl, C₃-C₆ cycloalkyl, 3- to 6-membered heterocycloalkyl, —CN, —OH, —OR²¹, —CO₂R²², —N(R²²)₂, —C(═O)N(R²²)₂, or —OC(═O)R²¹
or R¹³ and R¹⁴ are taken together with the carbon atom to which they are attached to form a —C(═O)—;
or R¹³ and R¹⁴ are taken together with the carbon atom to which they are attached to form a C₃-C₆ cycloalkyl or 3- to 6-membered heterocycloalkyl;
Y² is —(CR¹⁵R¹⁶)—, —O—, or —O—CR¹⁵R¹⁶—;
   each R¹⁵ and R¹⁶ is independently hydrogen, halogen, cyano, C₁-C₆ alkyl, C₁-C₆ fluoroalkyl, C₁-C₆ alkoxy, phenyl, monocyclic heteroaryl, C₃-C₆ cycloalkyl, or 3- to 6-membered heterocycloalkyl;
   or R¹⁴ and one R¹⁶ on an adjacent carbon atom are taken together to form an alkene bond;
   n is 1 or 2;
or R² is

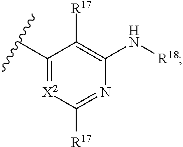

X² is CR¹⁷ or N;
each R¹⁷ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl. —CN. —OH, —OR²¹, —CO₂R²², —N(R²²)₂, —C(═O)N(R²²)₂, —NR²²C(═O)R²¹, —S(═O)₂N(R²²)₂, or —NR²²S(═O)₂R²¹;
R¹⁸ is substituted C₁-C₆ alkyl, unsubstituted or substituted C₁-C₆ fluoroalkyl, unsubstituted or substituted C₁-C₆ heteroalkyl, unsubstituted or substituted C₁-C₆ alkenyl, or unsubstituted or substituted C₁-C₆ alkynyl;
or R¹⁸ is unsubstituted or substituted cyclopropyl, unsubstituted or substituted cyclobutyl, unsubstituted or substituted cyclopentyl, or substituted cyclohexyl;
or R¹⁸ is unsubstituted or substituted C₅-C₁₀ bicyclic cycloalkyl;
or R¹⁸ is unsubstituted or substituted 3- to 10-membered monocyclic or bicyclic heterocycloalkyl;
or R¹⁸ is —CHR²⁰-(unsubstituted or substituted carbocycle) or —CHR²⁰-(unsubstituted or substituted heterocycle);
   R²⁰ is hydrogen or —CH₃;
R³ is hydrogen, unsubstituted or substituted C₁-C₆ alkyl, unsubstituted or substituted C₁-C₆ fluoroalkyl, unsubstituted or substituted C₁-C₆ alkoxy, unsubstituted or substituted phenyl, unsubstituted or substituted benzyl, unsubstituted or substituted monocyclic heteroaryl, unsubstituted or substituted C₃-C₆ cycloalkyl, or unsubstituted or substituted 3- to 6-membered heterocycloalkyl;
R⁴ is hydrogen or C₁-C₆ alkyl;
or R³ and R⁴ are taken together with the carbon atom to which they are attached to form a C₃-C₆ cycloalkyl or 3- to 6-membered heterocycloalkyl;
each R⁵ is independently hydrogen, halogen, unsubstituted or substituted C₁-C₆ alkyl, unsubstituted or substituted C₁-C₆ fluoroalkyl, —CN, —OH, —OR²¹, —CO₂R²², —N(R²²)₂, —C(═O)N(R²²)₂, —NR²²C(═O)R²², —S(═O)₂N(R²²)₂, or —NR²²S(═)₂R²¹;
each R²¹ is independently unsubstituted or substituted C₁-C₆ alkyl, unsubstituted or substituted C₃-C₆ cycloalkyl, unsubstituted or substituted 3- to 6-membered heterocycloalkyl, unsubstituted or substituted aryl, or unsubstituted or substituted heteroaryl;
each R²² is independently hydrogen, unsubstituted or substituted C₁-C₆ alkyl, unsubstituted or substituted C₃-C₆ cycloalkyl, unsubstituted or substituted 3- to 6-membered heterocycloalkyl, unsubstituted or substituted aryl, or unsubstituted or substituted heteroaryl; or two R² on the same N atom are taken together with the N atom to which they are attached to form an unsubstituted or substituted N-containing heterocycloalkyl;
wherein each substituted alkyl, substituted fluoroalkyl, substituted aryl, substituted heteroaryl, substituted cycloalkyl, and substituted heterocycloalkyl is substituted with one or more R^{s} groups independently selected from the group consisting of halogen, C₁-C₆ alkyl, C₁-C₆ fluoroalkyl, C₃-C₆ cycloalkyl, 3- to 6-membered heterocycloalkyl, phenyl, benzyl, 5-membered heteroaryl, 6-membered heteroaryl, —CN, —OR²³, —CO₂R²³, —C(═O)N(R²³)₂, —N(R²³)₂, —NR²³C(═O)R²⁴, —SR²³, —S(═O)R²⁴, —SO₂R²⁴, or —SO₂N(R²³)₂;
   each R²³ is independently selected from hydrogen, C₁-C₆ alkyl, C₃-C₆ cycloalkyl, 3- to 6-membered heterocycloalkyl, phenyl, benzyl, 5-membered heteroaryl and 6-membered heteroaryl; or two R²³ groups are taken together with the N atom to which they are attached to form a 3- to 6-membered N-containing heterocycloalkyl;
   each R²⁴ is independently selected from C₁-C₆ alkyl, C₃-C₆ cycloalkyl, 3- to 6-membered heterocycloalkyl, phenyl, benzyl, 5-membered heteroaryl and 6-membered heteroaryl.

In another aspect described herein is a compound of Formula (VIII):

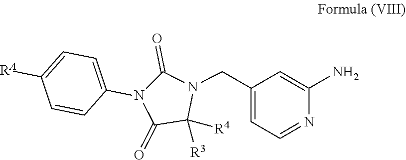

or a pharmaceutically acceptable salt or solvate thereof, wherein:
R^{A} is —SCF₃, —S(═O)CF₃, or —S(═O)₂CF₃;
R³ is C₂-C₆ alkyl which is unsubstituted or substituted by —OH, phenyl, benzyl, C₃-C₆ cycloalkyl, or —CH₂OH; and
R⁴ is hydrogen or methyl;
or R³ and R⁴ are taken together with the carbon atom to which they are attached to form a cyclobutyl, cyclopentyl, or cyclohexyl.

In another aspect described herein is a compound of Formula (IX):

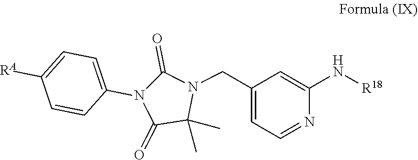

or a pharmaceutically acceptable salt or solvate thereof, wherein:
R^{A} is —SR^{A1}, —S(═O)R^{A1}, or —S(═O)₂R^{A1};
   R^{A1} is C₁-C₄ alkyl, C₁-C₄ fluoroalkyl, or C₃-C₆ cycloalkyl;
R¹⁸ is C₁-C₆ alkyl which is substituted by —CN, —OH, or —OMe;
or R¹⁹ is unsubstituted or substituted C₁-C₆ fluoroalkyl or unsubstituted or substituted C₁-C₆ heteroalkyl;
or R¹⁸ is unsubstituted or substituted 3- to 10-membered monocyclic or bicyclic heterocycloalkyl;
or R¹⁸ is —CHR²⁰-(unsubstituted or substituted C₃-C₆ cycloalkyl), —CHR²⁰-(unsubstituted or substituted 3- to 6-membered heterocycloalkyl), or —CHR²⁰-(unsubstituted or substituted 5-membered heteroaryl);
   R²⁰ is hydrogen or —CH₃;
or R¹⁸ is —C(═O)R¹⁹, where R¹⁹ is unsubstituted or substituted 3- to 10-membered heterocycloalkyl;
or R¹⁸ is —C(═O)NHR¹⁹, where R¹⁹ is unsubstituted or substituted C₃-C₁₀ cycloalkyl or unsubstituted or substituted 3- to 10-membered heterocycloalkyl;
wherein each substituted alkyl, substituted fluoroalkyl, substituted aryl, substituted heteroaryl, substituted cycloalkyl, and substituted heterocycloalkyl is substituted with one or more R^{s} groups independently selected from the group consisting of halogen, C₁-C₆ alkyl, C₁-C₆ fluoroalkyl, C₃-C₆ cycloalkyl, 3- to 6-membered heterocycloalkyl, phenyl, benzyl, 5-membered heteroaryl, 6-membered heteroaryl, —CN, —OR²³, —CO₂R²³, —C(═O)N(R²³)₂, —N(R²³)₂, —NR²³C(═O)R²⁴, —SR²³, —S(═O)R²⁴, —SO₂R²⁴, or —SO₂N(R²³)₂;
   each R²³ is independently selected from hydrogen, C₁-C₆ alkyl, C₃-C₆ cycloalkyl, 3- to 6-membered heterocycloalkyl, phenyl, benzyl, 5-membered heteroaryl and 6-membered heteroaryl; or two R²³ groups are taken together with the N atom to which they are attached to form a 3- to 6-membered N-containing heterocycloalkyl;
   each R²⁴ is independently selected from C₁-C₆ alkyl, C₃-C₆ cycloalkyl, 3- to 6-membered heterocycloalkyl, phenyl, benzyl, 5-membered heteroaryl and 6-membered heteroaryl.

Also described herein are pharmaceutical compositions comprising a compound described herein, or a pharmaceutically acceptable salt or solvate thereof, and at least one pharmaceutically acceptable excipient. In some embodiments, the pharmaceutical composition is formulated for administration to a mammal by intravenous administration, subcutaneous administration, oral administration, inhalation, nasal administration, dermal administration, or ophthalmic administration. In some embodiments, the pharmaceutical composition is formulated for administration to a mammal by oral administration. In some embodiments, the pharmaceutical composition is in the form of a tablet, a pill, a capsule, a liquid, a suspension, a gel, a dispersion, a solution, an emulsion, an ointment, or a lotion. In some embodiments, the pharmaceutical composition is in the form of a tablet, a pill, or a capsule.

In any of the aforementioned aspects are further embodiments in which the effective amount of the compound described herein, or a pharmaceutically acceptable salt thereof, is: (a) systemically administered to the mammal; and/or (b) administered orally to the mammal; and/or (c) intravenously administered to the mammal; and/or (d) administered by injection to the mammal; and/or (e) administered topically to the mammal; and/or (f) administered by ophthalmic administration; and/or (g) administered non-systemically or locally to the mammal.

In any of the aforementioned aspects are further embodiments comprising single administrations of the effective amount of the compound, including further embodiments in which the compound is administered once a day to the mammal or the compound is administered to the mammal multiple times over the span of one day. In some embodiments, the compound is administered on a continuous dosing schedule. In some embodiments, the compound is administered on a continuous daily dosing schedule.

In any of the embodiments disclosed herein, the mammal is a human.

In some embodiments, compounds provided herein are orally administered to a human.

Also described herein, in some embodiments, is a compound described herein, or a pharmaceutically acceptable salt or solvate thereof, for use as a medicament.

Also described herein, in some embodiments, is a compound described herein, or a pharmaceutically acceptable salt or solvate thereof, for use in the treatment of a disease or condition in a mammal that would benefit from the modulation of insulin-like growth factor-1 receptor (IGF-1R). In some embodiments, the disease or condition is dependent on IGF-1R activity. In some embodiments, the disease or condition is IGF-1R-mediated. In some embodiments, the disease or condition is selected from thyroid eye disease (TED), also known as thyroid-associated ophthalmopathy (TAO), or Graves' ophthalmopathy or orbitopathy (GO). In some embodiments, the disease or condition is thyroid eye disease (TED).

Also described herein, in some embodiments, is a compound described herein, or a pharmaceutically acceptable salt or solvate thereof, for use in the manufacture of a medicament for the prevention or treatment of a disease or condition ameliorated by the inhibition of IGF-1R.

Also described herein, in some embodiments, is a method of inhibition of IGF-1R comprising contacting IGF-1R with a compound described herein, or a pharmaceutically acceptable salt or solvate thereof.

Also described herein, in some embodiments, is a method of treatment of a IGF-1R-mediated disease comprising the administration of a therapeutically effective amount of a compound described herein, or a pharmaceutically acceptable salt or thereof, to a patient in need thereof. In some embodiments, said disease or condition is selected from thyroid eye disease (TED), also known as thyroid-associated ophthalmopathy (TAO), or Graves' ophthalmopathy or orbitopathy (GO). In some embodiments, said disease or condition is thyroid eye disease (TED).

Other objects, features and advantages of the compounds, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### DETAILED DESCRIPTION OF THE INVENTION

Compounds disclosed herein are IGF-1R inhibitors activity. In some embodiments, compounds disclosed herein are useful in the treatment of diseases or disorders where inhibition of IGF-1R activity is desired. Also provided is the use of certain compounds disclosed herein for use in the manufacture of a medicament for the treatment of a disease or condition ameliorated by the inhibition of IGF-1R.

### Compounds

Disclosed herein are compounds that inhibit IGF-1R activity. In some embodiments, the IGF-1R inhibitors described herein are useful in the treatment of diseases or disorders in which IGF-1R activity plays an active role or diseases or disorders which would benefit from inhibition of IGF-1R activity.

In one aspect, described herein is a compound of Formula (A):

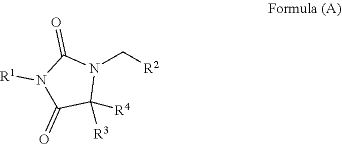

or a pharmaceutically acceptable salt or solvate thereof, wherein:
R¹ is unsubstituted or substituted phenyl, unsubstituted or substituted monocyclic heteroaryl, or unsubstituted or substituted bicyclic heteroaryl; wherein if R¹ is substituted, then it is substituted with R^{A} and 1-3 R⁵:
   R^{A} is unsubstituted or substituted C₁-C₆ alkyl, unsubstituted or substituted C₁-C₆ fluoroalkyl, unsubstituted or substituted phenyl, unsubstituted or substituted 5- or 6-membered heteroaryl, unsubstituted or substituted C₃-C₆ cycloalkyl, unsubstituted or substituted C₃-C₆ cycloalkenyl, unsubstituted or substituted 3- to 6-membered heterocycloalkyl, halogen, —CN, —SR^{A}, —S(═O)R^{A1}, —S(═O)₂R^{A1}, —OR^{A1}, —SF₅, or —SiMe₃;
   R^{A1} is C₁-C₆ alkyl, C₁-C₆ fluoroalkyl, C₃-C₆ cycloalkyl, or 3- to 6-membered heterocycloalkyl:
or R^{A} is

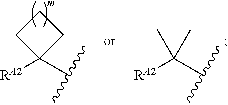

R^{A2} is halogen, —OH, —CN, C₁-C₄ alkyl, C₁-C₄ fluoroalkyl, C₁-C₄ alkoxy, C₁-C₄ fluoroalkoxyl, or C₁-C₄ hydroxyalkyl;
m is 0, 1, 2, or 3:
each R⁵ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²¹, —CO₂R²², —N(R²²)₂, —C(═O)N(R²²)₂, —NR²²C(═O)R²¹, —S(═O)₂N(R²²)₂, or —NR²²S(═O)₂R²¹;
or R¹ is

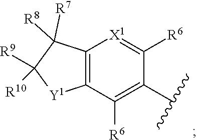

X¹ is CR⁶ or N;
   each R⁶ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²¹, —CO₂R²², —N(R²²)₂, —C(═O)N(R²²)₂, —NR²²C(═O)R²¹, —S(═O)₂N(R²²)₂, or —NR²²S(═O)₂R²¹;
Y¹ is —O—, —NR¹¹—, or —SO₂—;
   R¹¹ is hydrogen, C₁-C₆ alkyl, C₁-C₆ fluoroalkyl, —C(═O)NH₂, —C(═O)R¹², or —S(═O)₂R¹²;
   R¹² is unsubstituted or substituted C₁-C₆ alkyl, unsubstituted or substituted C₃-C₆ cycloalkyl, or unsubstituted or substituted 3- to 6-membered heterocycloalkyl:
R⁷ and R⁸ are each independently hydrogen, —OH, C₁-C₆ alkyl, C₁-C₆ fluoroalkyl, or C₁-C₆ alkoxy;
or R⁷ and R⁸ are taken together with the carbon atom to which they are attached to form an unsubstituted or substituted C₃-C₆ cycloalkyl or unsubstituted or substituted 3- to 6-membered heterocycloalkyl;
R⁹ and R¹⁰ are each independently hydrogen, halogen, C₁-C₆ alkyl, C₁-C₆ fluoroalkyl, or C₁-C₆ alkoxy;
or R⁹ and R¹⁰ are taken together with the carbon atom to which they are attached to form a —C(═O)—;
or R⁹ and R¹⁰ are taken together with the carbon atom to which they are attached to form an unsubstituted or substituted C₃-C₆ cycloalkyl or unsubstituted or substituted 3- to 6-membered heterocycloalkyl;
R² is

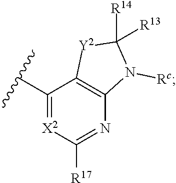

R^{c} is hydrogen, C₁-C₆ alkyl, C₁-C₆ fluoroalkyl, —C(═O)N(R²²)₂, —C(═O)R²¹, —C(═O)OR²², or —S(═O)₂R²¹;
X² is CR¹⁷ or N;
each R¹⁷ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²¹, —CO₂R²², —N(R²²)₂, —C(═O)N(R²²)₂, —OC(═O)R²¹, —NR²²C(═O)R²¹, —S(═O)₂N(R²²)₂, or —NR²²S(═O)₂R²¹;
R¹³ and R¹⁴ are each independently hydrogen, halogen, unsubstituted or substituted C₁-C₆ alkyl, C₁-C₆ fluoroalkyl, phenyl, monocyclic heteroaryl, C₃-C₆ cycloalkyl, 3- to 6-membered heterocycloalkyl, —CN, —OH, —OR²¹, —CO₂R²², —N(R²²)₂, —C(═O)N(R²²)₂, or —OC(═O)R²¹;
or R¹³ and R¹⁴ are taken together with the carbon atom to which they are attached to form a —C(═O)—;
or R¹³ and R¹⁴ are taken together with the carbon atom to which they are attached to form a C₃-C₆ cycloalkyl or 3- to 6-membered heterocycloalkyl;
Y² is —(CR¹⁵R¹⁶)ₙ—, —O—, or —O—CR¹⁵R¹⁶—;
   each R¹⁵ and R¹⁶ is independently hydrogen, halogen, cyano, C₁-C₆ alkyl, C₁-C₆ fluoroalkyl, C₁-C₆ alkoxy, phenyl, monocyclic heteroaryl, C₃-C₆ cycloalkyl, or 3- to 6-membered heterocycloalkyl;
   or R¹⁴ and one R on an adjacent carbon atom are taken together to form an alkene bond;
   n is 1 or 2;
or R² is

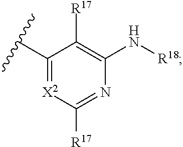

R¹⁸ is hydrogen, unsubstituted or substituted C₁-C₆ alkyl, unsubstituted or substituted C₁-C₆ fluoroalkyl, unsubstituted or substituted C₁-C₆ heteroalkyl, unsubstituted or substituted C₁-C₆ alkenyl, unsubstituted or substituted C₁-C₆ alkynyl, unsubstituted or substituted carbocycle, unsubstituted or substituted heterocycle, —CHR^{2Q}-(unsubstituted or substituted carbocycle), —CHR²⁰-(unsubstituted or substituted heterocycle), —C(═O)R¹⁹, —CO₂R¹⁹, or —C(═O)NHR¹⁹;
   R¹⁹ is hydrogen, unsubstituted or substituted C₁-C₆ alkyl, unsubstituted or substituted C₃-C₁₀ cycloalkyl, unsubstituted or substituted 3- to 10-membered heterocycloalkyl:
R²⁰ is hydrogen or —CH₃;
R³ is hydrogen, unsubstituted or substituted C₁-C₆ alkyl, unsubstituted or substituted C₁-C₆ fluoroalkyl, unsubstituted or substituted C₁-C₆ alkoxy, unsubstituted or substituted phenyl, unsubstituted or substituted benzyl, unsubstituted or substituted monocyclic heteroaryl, unsubstituted or substituted C₃-C₆ cycloalkyl, or unsubstituted or substituted 3- to 6-membered heterocycloalkyl;
R⁴ is hydrogen or C₁-C₆ alkyl;
or R³ and R⁴ are taken together with the carbon atom to which they are attached to form a C₃-C₆ cycloalkyl or 3- to 6-membered heterocycloalkyl;
each R²¹ is independently unsubstituted or substituted C₁-C₆ alkyl, unsubstituted or substituted C₃-C₆ cycloalkyl, unsubstituted or substituted 3- to 6-membered heterocycloalkyl, unsubstituted or substituted aryl, or unsubstituted or substituted heteroaryl;
each R²² is independently hydrogen, unsubstituted or substituted C₁-C₆ alkyl, unsubstituted or substituted C₃-C₆ cycloalkyl, unsubstituted or substituted 3- to 6-membered heterocycloalkyl, unsubstituted or substituted aryl, or unsubstituted or substituted heteroaryl; or two R²² on the same N atom are taken together with the N atom to which they are attached to form an unsubstituted or substituted N-containing heterocycloalkyl;
wherein each substituted alkyl, substituted fluoroalkyl, substituted aryl, substituted heteroaryl, substituted cycloalkyl, and substituted heterocycloalkyl is substituted with one or more R^{s} groups independently selected from the group consisting of halogen, C₁-C₆ alkyl, C₁-C₆ fluoroalkyl, C₃-C₆ cycloalkyl, 3- to 6-membered heterocycloalkyl, phenyl, benzyl, 5-membered heteroaryl, 6-membered heteroaryl. —CN, —OR²³, —CO₂R²³, —C(═O)N(R²³)₂, —N(R²³)₂, —NR²³C(═O)R²⁴, —SR²³, —S(═O)R²⁴, —SO₂R²⁴, or —SO₂N(R²³)₂;
   each R²³ is independently selected from hydrogen, C₁-C₆alkyl, C₃-C₆ cycloalkyl, 3- to 6-membered heterocycloalkyl, phenyl, benzyl, 5-membered heteroaryl and 6-membered heteroaryl; or two R²³ groups are taken together with the N atom to which they are attached to form a 3- to 6-membered N-containing heterocycloalkyl;
   each R²⁴ is independently selected from C₁-C₆ alkyl, C₃-C₆ cycloalkyl, 3- to 6-membered heterocycloalkyl, phenyl, benzyl, 5-membered heteroaryl and 6-membered heteroaryl.

For any and all of the embodiments, substituents are selected from among a subset of the listed alternatives. For example, in some embodiments, R⁴ is hydrogen or C₁-C₆ alkyl. In some embodiments, R⁴ is hydrogen or C₁-C₄ alkyl. In some embodiments, R⁴ is hydrogen or methyl. In some embodiments, R⁴ is methyl. In other embodiments, R⁴ is hydrogen.

In some embodiments, R³ is hydrogen, unsubstituted or substituted C₁-C₆ alkyl, unsubstituted or substituted C₁-C₆ fluoroalkyl, unsubstituted or substituted C₁-C₆ alkoxy, unsubstituted or substituted phenyl, unsubstituted or substituted benzyl, unsubstituted or substituted monocyclic heteroaryl, unsubstituted or substituted C₃-C₆ cycloalkyl, or unsubstituted or substituted 3- to 6-membered heterocycloalkyl. In some embodiments, R³ is hydrogen, unsubstituted or substituted C₁-C₆ alkyl, unsubstituted or substituted C₁-C₆ fluoroalkyl, unsubstituted or substituted phenyl, unsubstituted or substituted benzyl, or unsubstituted or substituted C₃-C₆ cycloalkyl. In some embodiments, R³ is unsubstituted or substituted C₁-C₆ alkyl, phenyl, benzyl, or C₃-C₆ cycloalkyl. In some embodiments, R³ is C₁—C₆ alkyl which is unsubstituted or substituted by —OH, phenyl, benzyl, or C₃-C₆ cycloalkyl. In some embodiments, R³ is methyl, ethyl, propyl, isopropyl, butyl, isobutyl, sec-butyl, phenyl, benzyl, cyclopropyl, cyclobutyl, cyclopentyl, cyclohexyl, or —CH₂OH. In some embodiments, R³ is C₂-C₆ alkyl which is unsubstituted or substituted by —OH, phenyl, benzyl, or C₃-C₆ cycloalkyl, or —CH₂OH. In some embodiments, R³ is ethyl, propyl, isopropyl, butyl, isobutyl, sec-butyl, phenyl, benzyl, cyclopropyl, cyclobutyl, cyclopentyl, cyclohexyl, or —CH₂OH.

In some embodiments, R³ and R⁴ are taken together with the carbon atom to which they are attached to form a C₃-C₆ cycloalkyl or 3- to 6-membered heterocycloalkyl. In some embodiments, R³ and R⁴ are taken together with the carbon atom to which they are attached to form a C₃-C₆ cycloalkyl. In some embodiments, R³ and R⁴ are taken together with the carbon atom to which they are attached to form a cyclopropyl, cyclobutyl, cyclopentyl, or cyclohexyl.

In some embodiments, R³ is C₁-C₆ alkyl; and R⁴ is C₁-C₆ alkyl; or R³ and R⁴ are taken together with the carbon atom to which they are attached to form a C₃-C₆ cycloalkyl.

In some embodiments, R¹ is unsubstituted or substituted phenyl, unsubstituted or substituted monocyclic heteroaryl, or unsubstituted or substituted bicyclic heteroaryl; wherein if R¹ is substituted, then it is substituted with R^{A} and 1-3 R³. In some embodiments, R¹ is unsubstituted or substituted phenyl or unsubstituted or substituted monocyclic heteroaryl; wherein if R¹ is substituted, then it is substituted with R^{A} and 1-3 R⁵. In some embodiments, R¹ is unsubstituted or substituted bicyclic heteroaryl, wherein if R¹ is substituted, then it is substituted with R^{A} and 1-3 R⁵.

In some embodiments, R¹ is

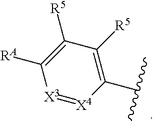

In some embodiments, each R⁵ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²¹, —CO₂R²², —N(R²²)₂, —C(═O)N(Rn)²², —NR²²C(═O)R²¹, —S(═O)₂N(R²²)₂, or —NR²²S(═O)₂R²¹. In some embodiments, each R⁵ is independently hydrogen, halogen, unsubstituted or substituted C₁-C₆ alkyl, unsubstituted or substituted C₁-C₆ fluoroalkyl, unsubstituted or substituted C₃-C₆ cycloalkyl, unsubstituted or substituted 3- to 6-membered heterocycloalkyl, —CN, —OH, —OR²¹, —N(R²²)₂, or —C(═O)N(R²²)₂. In some embodiments, each R⁵ is independently hydrogen, halogen, C₁-C₆ alkyl, C₁-C₆ fluoroalkyl, C₃-C₆ cycloalkyl, 3- to 6-membered heterocycloalkyl, —CN, or —OR²¹. In some embodiments, each R⁵ is independently hydrogen or C₁-C₆ alkyl. In some embodiments, each R⁵ is hydrogen.

In some embodiments, R¹ is

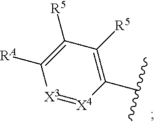

wherein X³ and X⁴ are each independently CR⁵ or N; and each R⁵ is independently hydrogen, halogen, C₁-C₆ alkyl, C₁-C₆ fluoroalkyl, C₃-C₆ cycloalkyl, 3- to 6-membered heterocycloalkyl, —CN, or —OR²¹. In some embodiments, X³ and X⁴ are each CR⁵; and each R⁵ is hydrogen.

In some embodiments, R^{A} is C₁-C₆ alkyl, C₁-C₆ fluoroalkyl, halogen, —CN, —SR^{A1}, —S(═O)R^{A}, —S(═O)₂R^{A}1, —OR^{A1}—SF₅, or —SiMe₃; R^{A} is C₁-C₄ alkyl, or C₁-C₄ fluoroalkyl; or R^{A} is

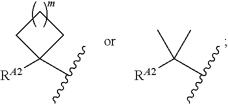

R^{A2} is halogen, —CN, C₁-C₄ alkyl, or C₁-C₄ fluoroalkyl; and m is 1. In some embodiments, R^{A} is —CH₃, —CH₂CH₃, —CH(CH₃)₂, —C(CH₃)₃, —CF₃, —F, —C₁, —Br, —CN, —SCF₃, —S(═O)CF₃, —S(═O)₂CF₃, —S(═O)₂CHF₂, —S(═O)₂C(CH₃)₃, —S(═O)₂-(cyclopropyl), —SF₅, or —SiMe₃; or R^{A} is

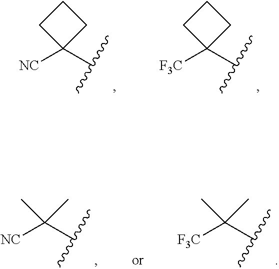

In some embodiments, R^{A} is unsubstituted or substituted C₁-C₆ alkyl, unsubstituted or substituted C₁-C₆ fluoroalkyl, unsubstituted or substituted phenyl, unsubstituted or substituted 5- or 6-membered heteroaryl, unsubstituted or substituted C₃-C₆ cycloalkyl, unsubstituted or substituted C₃-C₆ cycloalkenyl, unsubstituted or substituted 3- to 6-membered heterocycloalkyl, halogen. —CN, —SR^{A1}, —S(═O)R^{A1}, —S(═O)₂R^{A}, —SF₅, or —SiMe₃.

In some embodiments, R^{A} is

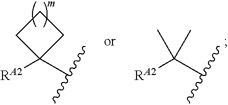

R^{A2} is halogen, —OH, —CN, C₁-C₄ alkyl, C₁-C₄ fluoroalkyl, C₁-C₄ alkoxy, C₁-C₄ fluoroalkoxyl, or C₁-C₄ hydroxyalkyl; and m is 0, 1, 2, or 3.

In some embodiments, R^{A} is unsubstituted or substituted C₁-C₆ fluoroalkyl, unsubstituted or substituted phenyl, unsubstituted or substituted 5- or 6-membered heteroaryl, unsubstituted or substituted C₃-C₆ cycloalkyl, unsubstituted or substituted C₃-C₆ cycloalkenyl, or unsubstituted or substituted 3- to 6-membered heterocycloalkyl, halogen, —CN, —SF₅, or —SiMe₃.

In some embodiments, R^{A} is unsubstituted or substituted C₁-C₆ alkyl, unsubstituted or substituted C₁-C₆ fluoroalkyl, unsubstituted or substituted C₃-C₆ cycloalkyl, unsubstituted or substituted 3- to 6-membered heterocycloalkyl, halogen, —CN, —SR^{A1}, —S(═O)R^{A1}, —S(═O)₂R^{A}, —OR^{A1}, —SF₅, or —SiMe₃.

In some embodiments, R^{A} is unsubstituted or substituted C₁-C₆ alkyl.

In some embodiments, R^{A} is —SR^{A1}, —S(═O)R^{A1}, or —S(═O)₂R^{A1}; and R^{A1} is C₁-C₄ alkyl, C₁-C₄ fluoroalkyl, or C₃-C₆ cycloalkyl. In some embodiments, R^{A} is —SCF₃, —S(═O)CF₃, —S(═O)₂CF₃, —S(═O)₂CHF₂, —S(═O)₂C(CH₃)₃, or —S(═O)₂-(cyclopropyl). In some embodiments, R^{A} is —SCF₃, —S(═O)CF₃, or —S(═O)₂CF₃.

In some embodiments, R¹ is

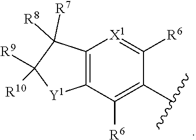

In some embodiments, X¹ is CR⁶ or N. In some embodiments, X¹ is CR⁶. In some embodiments. X¹ is N.

In some embodiments, Y¹ is —O—, —NR¹¹—, or —SO₂—. In some embodiments, Y¹ is —O— or —NR¹¹—. In some embodiments, Y¹ is —O—. In some embodiments, Y¹ is —NR¹¹—. In some embodiments, Y¹ is —SO₂—.

In some embodiments, R¹¹ is hydrogen, C₁-C₆ alkyl, C₁-C₆ fluoroalkyl, —C(═O)NH₂, —C(═O)R¹¹, or —S(═O)₂R¹². In some embodiments, R¹¹ is hydrogen or C₁-C₆ alkyl. In some embodiments, R¹¹ is hydrogen. In some embodiments, R¹¹ is —C(═O)NH₂, —C(═O)R¹², or —S(═O)₂R¹².

In some embodiments, R¹² is unsubstituted or substituted C₁-C₆ alkyl, unsubstituted or substituted C₃-C₆ cycloalkyl, or unsubstituted or substituted 3- to 6-membered heterocycloalkyl. In some embodiments, R¹² is unsubstituted or substituted C₁-C₆ alkyl or unsubstituted or substituted C₃-C₆ cycloalkyl. In some embodiments, R¹² is C₁-C₆ alkyl or C₃-C₆ cycloalkyl. In some embodiments, R¹² is C₁-C₆ alkyl. In some embodiments, R² is C₃-C₆ cycloalkyl.

In some embodiments, each R⁶ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²¹, —CO₂R²², —N(R²²)₂, —C(═O)N(R²²)₂, —NR²²C(═O)R²¹, —S(═O)₂N(R²²)₂, or —NR²²S(═O)₂R²¹. In some embodiments, each R⁶ is independently hydrogen, halogen, C₁-C₆ alkyl, C₁-C₆ fluoroalkyl, unsubstituted or substituted C₃-C₆ cycloalkyl, unsubstituted or substituted 3- to 6-membered heterocycloalkyl, —CN, —OH, —OR²¹, —CO₂R²², —N(R²²)₂, —C(═O)N(R²²)₂, —NR²²C(═O)R²¹, —S(═O)₂N(R²²)₂, or —NR²²S(═O)₂R²¹. In some embodiments, each R⁶ is independently hydrogen, halogen, C₁-C₆ alkyl, C₁-C₆ fluoroalkyl, —CN, —OH, —OR²¹, —N(R²)₂, or —C(═O)N(R²²)₂. In some embodiments, each R⁶ is independently hydrogen, halogen, or C₁-C₆ alkyl. In some embodiments, each R⁶ is hydrogen.

In some embodiments, R⁷ and R⁸ are each independently hydrogen. —OH, C₁-C₆ alkyl, C₁-C₆ fluoroalkyl, or C₁-C₆ alkoxy. In some embodiments, R⁷ and R⁸ are each independently hydrogen, —OH, or C₁-C₆ alkyl. In some embodiments, R⁷ and R⁸ are each independently hydrogen or C₁-C₆ alkyl. In some embodiments, R⁷ and R⁸ are each independently hydrogen or methyl.

In some embodiments, R⁷ and R⁸ are taken together with the carbon atom to which they are attached to form an unsubstituted or substituted C₃-C₆ cycloalkyl or unsubstituted or substituted 3- to 6-membered heterocycloalkyl. In some embodiments, R⁷ and R⁸ are taken together with the carbon atom to which they are attached to form an unsubstituted or substituted C₃-C₆ cycloalkyl. In some embodiments, R⁷ and R⁸ are taken together with the carbon atom to which they are attached to form an unsubstituted or substituted C₃-C₄ cycloalkyl. In some embodiments, R⁷ and R¹ are taken together with the carbon atom to which they are attached to form a 3- to 6-membered heterocycloalkyl. In some embodiments, R⁷ and R⁸ are taken together with the carbon atom to which they are attached to form a 3- or 4-membered heterocycloalkyl.

In some embodiments, R⁹ and R¹⁰ are each independently hydrogen, halogen, C₁-C₆ alkyl, C₁-C₆ fluoroalkyl, or C₁-C₆ alkoxy. In some embodiments, R⁹ and R¹⁰ are each independently hydrogen, halogen, C₁-C₆ alkyl, or C₁-C₆ fluoroalkyl. In some embodiments, R⁹ and R¹⁰ are each independently hydrogen or C₁-C₆ alkyl. In some embodiments, R⁹ and R¹⁰ are each independently hydrogen or methyl.

In some embodiments, R⁹ and R¹⁰ are taken together with the carbon atom to which they are attached to form a —C(═O)—.

In some embodiments, R⁹ and R¹⁰ are taken together with the carbon atom to which they are attached to form an unsubstituted or substituted C₃-C₆ cycloalkyl or unsubstituted or substituted 3- to 6-membered heterocycloalkyl. In some embodiments, R⁹ and R¹⁰ are taken together with the carbon atom to which they are attached to form an unsubstituted or substituted C₃-C₆ cycloalkyl. In some embodiments, R⁹ and R¹⁰ are taken together with the carbon atom to which they are attached to form an unsubstituted or substituted C₃-C₄ cycloalkyl. In some embodiments, R⁹ and R¹⁰ are taken together with the carbon atom to which they are attached to form a 3- to 6-membered heterocycloalkyl. In some embodiments, R⁹ and R¹⁰ are taken together with the carbon atom to which they are attached to form a 3- or 4-membered heterocycloalkyl.

In some embodiments, R¹ is

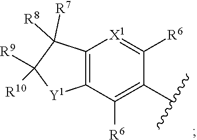

X¹ is CR⁶ or N; and each R⁶ is independently hydrogen, halogen, C₁-C₆ alkyl, C₁-C₆ fluoroalkyl, unsubstituted or substituted C₃-C₆ cycloalkyl, unsubstituted or substituted 3- to 6-membered heterocycloalkyl, —CN, —OH, —OR²¹, —CO₂R²², —N(R²²)₂, —C(═O)N(R²²)₂, —NR²²C(═O)R²¹, —S(═O)₂N(R²²)₂, or —NR²²S(═O)₂R²¹.

In some embodiments, Y¹ is —NR¹¹—, R¹¹ is hydrogen, C₁-C₆ alkyl, C₁-C₆ fluoroalkyl, —C(═O)NH₂, —C(═O)R¹², or —S(═O)₂R¹²; and R¹² is unsubstituted or substituted C₁-C₆ alkyl or unsubstituted or substituted C₃-C₆ cycloalkyl.

In some embodiments, R⁷ and R⁸ are each independently hydrogen, —OH, C₁-C₆ alkyl, C₁-C₆ fluoroalkyl, or C₁-C₆ alkoxy; or R⁷ and R⁸ are taken together with the carbon atom to which they are attached to form an unsubstituted or substituted C₃-C₄ cycloalkyl; R⁹ and R⁰ are each independently hydrogen, halogen, C₁-C₆ alkyl, C₁-C₆ fluoroalkyl, or C₁-C₆ alkoxy; or R⁹ and R¹⁰ are taken together with the carbon atom to which they are attached to form a —C(═O)—; or R⁹ and R¹⁰ are taken together with the carbon atom to which they are attached to form an unsubstituted or substituted C₃-C₄ cycloalkyl.

In some embodiments, R² is

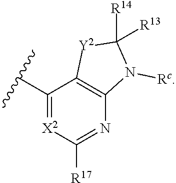

In some embodiments, R is hydrogen, C₁-C₆ alkyl, C₁-C₆ fluoroalkyl, —C(═O)N(R²²)₂, —C(═O)R²¹, —C(═O)OR²², or —S(═O)₂R²¹. In some embodiments. R is hydrogen, C₁-C₆ alkyl, C₁-C₆ fluoroalkyl, —C(═O)N(R²²)₂, —C(═O)R²¹, or —C(═)OR²². In some embodiments, R^{c} is hydrogen, C₁-C₆ alkyl, C₁-C₆ fluoroalkyl, or —C(═O)OR²². In some embodiments, R^{c} is hydrogen, C₁-C₆ alkyl, or —C(═O)OR²². In some embodiments, R^{c} is hydrogen, C₁-C₆ alkyl, or C₁-C₆ fluoroalkyl. In some embodiments, R is hydrogen or C₁-C₆ alkyl. In some embodiments, R^{c} is hydrogen.

In some embodiments, X² is CR¹⁷ or N. In some embodiments, X² is CR¹⁷. In some embodiments, X² is N.

In some embodiments, Y² is —(CR¹⁵R¹⁶)ₙ—, —O—, or —O—CR¹⁵R¹⁶—. In some embodiments, Y² is —(CR¹⁵R¹⁶)ₙ—, and n is 1 or 2. In some embodiments, Y² is —(CR¹⁵R¹⁶)— or —(CR¹⁵R¹⁶)—(CR¹⁵R¹⁶)—. In some embodiments, Y² is —O—. In some embodiments, Y² is —O—CR¹⁵R¹⁶—.

In some embodiments, R¹³ and R¹⁴ are each independently hydrogen, halogen, unsubstituted or substituted C₁-C₆ alkyl, C₁-C₆ fluoroalkyl, phenyl, monocyclic heteroaryl, C₃-C₆ cycloalkyl, 3- to 6-membered heterocycloalkyl, —CN, —OH, —OR²¹, —CO₂R²², —N(R²²)₂, —C(═O)N(R²²)₂, or —OC(═O)R²¹. In some embodiments, R¹³ and R¹⁴ are each independently hydrogen, halogen, C₁-C₆ alkyl, C₁-C₆ fluoroalkyl, C₃-C₆ cycloalkyl, or 3- to 6-membered heterocycloalkyl. In some embodiments, R¹³ and R¹⁴ are each independently hydrogen or C₁-C₆ alkyl. In some embodiments, R¹³ and R¹⁴ are each hydrogen.

In some embodiments, R¹ and R¹⁴ are taken together with the carbon atom to which they are attached to form a —C(═O)—.

In some embodiments, R¹³ and R¹⁴ are taken together with the carbon atom to which they are attached to form a C₃-C₆ cycloalkyl or 3- to 6-membered heterocycloalkyl. In some embodiments, R¹³ and R¹⁴ are taken together with the carbon atom to which they are attached to form a C₃-C₆ cycloalkyl. In some embodiments, R¹³ and R¹⁴ are taken together with the carbon atom to which they are attached to form a C₃-C₄ cycloalkyl. In some embodiments, R¹³ and R¹⁴ are taken together with the carbon atom to which they are attached to form a 3- to 6-membered heterocycloalkyl. In some embodiments, R¹³ and R¹⁴ are taken together with the carbon atom to which they are attached to form a 3- or 4-membered heterocycloalkyl.

In some embodiments, each R¹⁵ and R¹⁶ is independently hydrogen, halogen, cyano, C₁-C₆ alkyl, C₁-C₆ fluoroalkyl, C₁-C₆ alkoxy, phenyl, monocyclic heteroaryl, C₃-C₆ cycloalkyl, or 3- to 6-membered heterocycloalkyl. In some embodiments, each R¹⁵ and R¹⁶ is independently hydrogen, halogen, C₁-C₆ alkyl, C₁-C₆ fluoroalkyl, or C₃-C₆ cycloalkyl. In some embodiments, each R¹⁵ and R¹⁶ is independently hydrogen, C₁-C₆ alkyl, C₁-C₆ fluoroalkyl, or C₃-C₆ cycloalkyl. In some embodiments, each R¹⁵ and R¹⁶ is independently hydrogen or C₁-C₆ alkyl. In some embodiments, each R¹⁵ and R¹⁶ is independently hydrogen.

In some embodiments, R¹⁴ and one R¹⁶ on an adjacent carbon atom are taken together to form an alkene bond.

In some embodiments, each R¹⁷ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR^{2′}, —CO₂R²², —N(R²²)₂, —C(═O)N(R²)₂, —OC(═O)R²¹, —NR²²C(═O)R²¹, —S(═O)₂N(R²²)₂, or —NR²²S(═O)₂R²¹. In some embodiments, each R¹⁷ is independently hydrogen, halogen, unsubstituted or substituted C₁-C₆ alkyl, unsubstituted or substituted C₁-C₆ fluoroalkyl, —CN, —OH, —OR²¹, —CO₂R²², —N(R²²)₂, —C(═O)N(R²²)₂, —OC(═O)R²¹, —NR²²C(═O)R²¹, —S(═O)₂N(R²²)₂, or —NR²²S(═O)₂R²¹. In some embodiments, each R¹⁷ is independently hydrogen, halogen, C₁-C₆ alkyl, C₁-C₆ fluoroalkyl, —CN, —OH, —OR², —N(R²²)₂, or —C(═O)N(R²²)₂. In some embodiments, each R¹⁷ is independently hydrogen, halogen, C₁-C₆ alkyl, or C₁-C₆ fluoroalkyl. In some embodiments, each R¹⁷ is hydrogen.

In some embodiments, R is hydrogen, C₁-C₆ alkyl, or —C(═O)OR²²; X² is CR¹⁷; each R¹⁷ is independently hydrogen, halogen, C₁-C₆ alkyl, or C₁-C₆ fluoroalkyl; R¹³ and R¹⁴ are each independently hydrogen, halogen, C₁-C₆ alkyl, C₁-C₆ fluoroalkyl, C₃-C₆ cycloalkyl, or 3- to 6-membered heterocycloalkyl; or R¹³ and R⁴ are taken together with the carbon atom to which they are attached to form a —C(═O)—; or R¹³ and R¹⁴ are taken together with the carbon atom to which they are attached to form a C₃-C₄ cycloalkyl; Y² is —(CR¹⁵R¹⁶)ₙ—, —O—, or —O—CR¹⁵R¹⁶—; and each R¹⁵ and R¹⁶ is independently hydrogen, halogen, C₁-C₆ alkyl, C₁-C₆ fluoroalkyl, or C₃-C₆ cycloalkyl; or R¹⁴ and one R¹⁶ on an adjacent carbon atom are taken together to form an alkene bond.

In some embodiments, R^{c} is hydrogen; X² is CR¹⁷; each R¹⁷ is hydrogen; R¹³ and R¹⁴ are each hydrogen; or R¹³ and R¹⁴ are taken together with the carbon atom to which they are attached to form a —C(═O)—; and Y² is —(CR¹⁵R¹⁶)ₙ—, —O—, or —O—CR¹⁵R¹⁶—. In some embodiments, each R¹⁵ and R¹⁶ is independently hydrogen, C₁-C₆ alkyl, C₁-C₆ fluoroalkyl, or C₃-C₆ cycloalkyl.

In some embodiments,

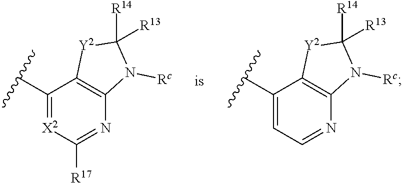

and Y² is —CR¹⁵R¹⁶— or —CR¹⁵R¹⁶—CR¹⁵R¹⁶—. In some embodiments,

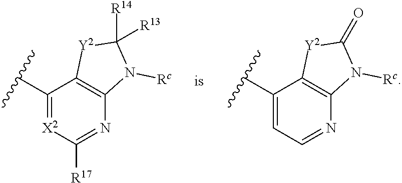

In some embodiments, R¹⁴ and one R¹⁶ on an adjacent carbon atom are taken together to form an alkene bond.

In some embodiments, R² is

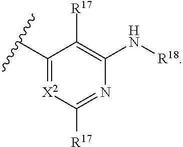

In some embodiments, X² is CR¹⁷ or N. In some embodiments, X² is CR¹⁷. In some embodiments, X² is N.

In some embodiments, each R¹⁷ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²¹, —CO₂R²², —N(R²²)₂, —C(═O)N(R²²)₂, —OC(═O)R²¹, —NR²²C(═O)R²¹. —S(═O)₂N(R²²)₂, or —NR²²S(═O)₂R²¹. In some embodiments, each R¹⁷ is independently hydrogen, halogen, unsubstituted or substituted C₁-C₆ alkyl, unsubstituted or substituted C₁-C₆ fluoroalkyl, —CN, —OH, —OR²¹, —CO₂R²², —N(R²²)₂, —C(═O)N(R²²)₂, —OC(═O)R²¹, —NR²C(═O)R²¹, —S(═O)₂N(R²²)₂, or —NR²S(═O)₂R²¹. In some embodiments, each R¹⁷ is independently hydrogen, halogen, C₁-C₆ alkyl, C₁-C₆ fluoroalkyl, —CN, —OH, —OR²¹—N(R²²)₂, or —C(═O)N(R²²)₂. In some embodiments, each R¹⁷ is independently hydrogen, halogen, C₁-C₆ alkyl, or C₁-C₆ fluoroalkyl. In some embodiments, each R¹⁷ is hydrogen.

In some embodiments, X² is CR¹⁷; and each R¹⁷ is independently hydrogen, halogen, C₁-C₆ alkyl, or C₁-C₆ fluoroalkyl.

In some embodiments, X² is CR¹⁷; and each R⁷ is hydrogen.

In some embodiments, R¹⁸ is hydrogen, unsubstituted or substituted C₁-C₆ alkyl, unsubstituted or substituted C₁-C₆ fluoroalkyl, unsubstituted or substituted C₁-C₆ heteroalkyl, unsubstituted or substituted C₁-C₆ alkenyl, unsubstituted or substituted C₁-C₆ alkynl, unsubstituted or substituted carbocycle, unsubstituted or substituted heterocycle, —CHR²⁰-(unsubstituted or substituted carbocycle), —CHR²⁰-(unsubstituted or substituted heterocycle), —C(═O)R¹⁹, —CO₂R¹⁹, or —C(═O)NHR¹⁹.

In some embodiments, R¹⁸ is hydrogen, unsubstituted or substituted C₁-C₆ alkyl, unsubstituted or substituted C₁-C₆ fluoroalkyl, unsubstituted or substituted C₁-C₆ heteroalkyl, unsubstituted or substituted C₁-C₆ alkenyl, unsubstituted or substituted C₁-C₆ alkynyl. In some embodiments, R¹⁸ is substituted C₁-C₆ alkyl, unsubstituted or substituted C₁-C₆ fluoroalkyl, or unsubstituted or substituted C₁-C₆ heteroalkyl. In some embodiments, R¹⁸ is C₁-C₆ alkyl which is substituted by —CN, —OH, or —OMe. In some embodiments, R¹⁸ is hydrogen.

In some embodiments, R¹⁸ is unsubstituted or substituted carbocycle or unsubstituted or substituted heterocycle. In some embodiments, R¹⁸ is unsubstituted or substituted C₃-C₁₀ cycloalkyl, unsubstituted or substituted 3- to 10-membered heterocycloalkyl, unsubstituted or substituted aryl, or unsubstituted or substituted heteroaryl. In some embodiments, R¹⁸ is unsubstituted or substituted C₃-C₆ monocyclic cycloalkyl, unsubstituted or substituted C₅-C₁₀ bicyclic cycloalkyl, or unsubstituted or substituted 3- to 10-membered monocyclic or bicyclic heterocycloalkyl. In some embodiments, R¹⁸ is unsubstituted or substituted 3- to 10-membered monocyclic or bicyclic heterocycloalkyl containing an oxygen atom; or R¹⁸ is unsubstituted or substituted 5- to 10-membered bicyclic heterocycloalkyl which is a bridged, fused, or spirocyclic heterocycloalkyl.

In some embodiments, R¹⁸ is —CHR²-(unsubstituted or substituted carbocycle) or —CHR⁰-(unsubstituted or substituted heterocycle). In some embodiments, R¹⁸ is —CHR²⁰-(unsubstituted or substituted C₃-C₆ cycloalkyl), —CHR²⁰-(unsubstituted or substituted 3- to 6-membered heterocycloalkyl), —CHR²⁰-(unsubstituted or substituted phenyl), or —CHR²⁰-(unsubstituted or substituted 5- or 6-membered heteroaryl); and R²⁰ is hydrogen or —CH₃.

In some embodiments, R¹⁸ is —C(═O)R¹⁹, —CO₂R¹⁹, or —C(═O)NHR¹⁹; and R¹⁹ is unsubstituted or substituted C₁-C₆ alkyl, unsubstituted or substituted C₃-C₁₀ cycloalkyl, unsubstituted or substituted 3- to 10-membered heterocycloalkyl.

In some embodiments, R¹⁸ is unsubstituted or substituted C₁-C₆ alkyl, unsubstituted or substituted C₁-C₆ fluoroalkyl, unsubstituted or substituted C₁-C₆ heteroalkyl, unsubstituted or substituted C₁-C₆ alkenyl, unsubstituted or substituted C₁-C₆ alkynyl, unsubstituted or substituted C₃-C₁₀ cycloalkyl, unsubstituted or substituted 3- to 10-membered heterocycloalkyl, —CHR²⁰-(unsubstituted or substituted carbocycle), —CHR²-(unsubstituted or substituted heterocycle), —C(═O)R¹⁹, —CO₂R¹⁹, or —C(═O)NHR¹⁹.

In some embodiments, R¹⁸ is unsubstituted or substituted C₃-C₁₀ cycloalkyl or unsubstituted or substituted 3- to 10-membered heterocycloalkyl. In some embodiments, R¹⁸ is unsubstituted or substituted C₃-C₆ monocyclic cycloalkyl, unsubstituted or substituted C₅-C₁₀ bicyclic cycloalkyl, or unsubstituted or substituted 3- to 10-membered monocyclic or bicyclic heterocycloalkyl. In some embodiments, R¹¹ is unsubstituted or substituted 3- to 10-membered monocyclic or bicyclic heterocycloalkyl containing an oxygen atom; or R¹⁸ is unsubstituted or substituted 5- to 10-membered bicyclic heterocycloalkyl which is a bridged, fused, or spirocyclic heterocycloalkyl.

In some embodiments, R¹⁸ is unsubstituted or substituted C₁-C₆ alkyl, unsubstituted or substituted C₁-C₆ fluoroalkyl, unsubstituted or substituted C₁-C₆ heteroalkyl, unsubstituted or substituted C₁-C₆ alkenyl, or unsubstituted or substituted C₁-C₆ alkynyl; or R¹⁸ is substituted cyclopropyl, unsubstituted or substituted cyclobutyl, unsubstituted or substituted cyclopentyl, or unsubstituted or substituted cyclohexyl; or R¹⁸ is unsubstituted or substituted C₅-C₁₀ bicyclic cycloalkyl; or R¹⁸ is unsubstituted or substituted 3- to 10-membered monocyclic or bicyclic heterocycloalkyl containing an oxygen atom; or R¹⁸ is —CHR²⁰-(unsubstituted or substituted carbocycle) or —CHR²⁰-(unsubstituted or substituted heterocycle); R²⁰ is hydrogen or —CH₃:

In some embodiments, R¹⁸ is substituted cyclopropyl, unsubstituted or substituted cyclobutyl, unsubstituted or substituted cyclopentyl, or unsubstituted or substituted cyclohexyl.

In some embodiments, R¹⁸ is unsubstituted or substituted C₅-C₁₀ bicyclic cycloalkyl. In some embodiments, R¹⁸ is C₅-C₁₀ cycloalkyl which is a bridged, fused, or spirocyclic cycloalkyl.

In some embodiments, R¹⁸ is unsubstituted or substituted 3- to 10-membered monocyclic or bicyclic heterocycloalkyl containing an oxygen atom. In some embodiments, R¹⁸ is unsubstituted or substituted 3- to 10-membered monocyclic or bicyclic heterocycloalkyl containing an oxygen atom which is a bridged, fused, or spirocyclic heterocycloalkyl.

In some embodiments, R¹⁸ is —CHR²⁰-(unsubstituted or substituted carbocycle) or —CHR²⁰-(unsubstituted or substituted heterocycle). In some embodiments, R¹⁸ is —CHR²⁰-(unsubstituted or substituted C₃-C₆ cycloalkyl), —CHR²⁰-(unsubstituted or substituted 3- to 6-membered heterocycloalkyl), —CHR²⁰-(unsubstituted or substituted phenyl), or —CHR²⁰-(unsubstituted or substituted 5- or 6-membered heteroaryl); and R²¹ is hydrogen or —CH₃.

In some embodiments, R¹⁸ is substituted C₁-C₆ alkyl, unsubstituted or substituted C₁-C₆ fluoroalkyl, unsubstituted or substituted C₁-C₆ heteroalkyl, unsubstituted or substituted C₁-C₆ alkenyl, or unsubstituted or substituted C₁-C₆ alkynyl; or R¹⁸ is unsubstituted or substituted cyclopropyl, unsubstituted or substituted cyclobutyl, unsubstituted or substituted cyclopentyl, or substituted cyclohexyl; or R¹⁸ is unsubstituted or substituted C₅-C₁₀ bicyclic cycloalkyl; or R¹⁸ is unsubstituted or substituted 3- to 10-membered monocyclic or bicyclic heterocycloalkyl; or R¹⁸ is —CHR²⁰-(unsubstituted or substituted carbocycle) or —CHR²-(unsubstituted or substituted heterocycle); and R²⁰ is hydrogen or —CH₃.

In some embodiments, R¹⁸ is substituted C₁-C₆ alkyl, unsubstituted or substituted C₁-C₆ fluoroalkyl, unsubstituted or substituted C₁-C₆ heteroalkyl, unsubstituted or substituted C₁-C₆ alkenyl, unsubstituted or substituted C₁-C₆ alkynyl. In some embodiments, R¹⁸ is substituted C₁-C₆ alkyl, unsubstituted or substituted C₁-C₆ fluoroalkyl, unsubstituted or substituted C₁-C₆ heteroalkyl. In some embodiments, R¹⁸ is C₁-C₆ alkyl which is substituted by —CN, —OH, or —OMe.

In some embodiments, R¹⁸ is unsubstituted or substituted cyclopropyl, unsubstituted or substituted cyclobutyl, unsubstituted or substituted cyclopentyl, or substituted cyclohexyl.

In some embodiments, R¹⁸ is unsubstituted or substituted C₅-C₁₀ bicyclic cycloalkyl which is a bridged, fused, or spirocyclic heterocycloalkyl.

In some embodiments, R¹⁸ is unsubstituted or substituted 3- to 10-membered monocyclic or bicyclic heterocycloalkyl.

In some embodiments, R¹⁸ is unsubstituted or substituted 3- to 10-membered monocyclic or bicyclic heterocycloalkyl containing an oxygen atom:

In some embodiments, R¹⁸ is unsubstituted or substituted 5- to 10-membered bicyclic heterocycloalkyl which is a bridged, fused, or spirocyclic heterocycloalkyl.

In some embodiments, R¹⁸ is —CHR²⁰-(unsubstituted or substituted C₃-C₆ cycloalkyl), —CHR²⁰-(unsubstituted or substituted 3- to 6-membered heterocycloalkyl), —CHR²⁰-(unsubstituted or substituted phenyl), or —CHR²⁰-(unsubstituted or substituted 5- or 6-membered heteroaryl); and R² is hydrogen or —CH₃.

In some embodiments, each R²¹ is independently unsubstituted or substituted C₁-C₆ alkyl, unsubstituted or substituted C₃-C₆ cycloalkyl, unsubstituted or substituted 3- to 6-membered heterocycloalkyl, unsubstituted or substituted aryl, or unsubstituted or substituted heteroaryl. In some embodiments, each R²¹ is independently unsubstituted or substituted C₁-C₆ alkyl, unsubstituted or substituted C₃-C₆ cycloalkyl, or unsubstituted or substituted 3- to 6-membered heterocycloalkyl. In some embodiments, each R²¹ is independently C₁-C₆ alkyl, C₃-C₆ cycloalkyl, or 3- to 6-membered heterocycloalkyl. In some embodiments, each R²¹ is independently C₁-C₆ alkyl or C₃-C₆ cycloalkyl. In some embodiments, each R²¹ is independently C₁-C₆ alkyl.

In some embodiments, each R²² is independently hydrogen, unsubstituted or substituted C₁-C₆ alkyl, unsubstituted or substituted C₃-C₆ cycloalkyl, unsubstituted or substituted 3- to 6-membered heterocycloalkyl, unsubstituted or substituted aryl, or unsubstituted or substituted heteroaryl; or two R² on the same N atom are taken together with the N atom to which they are attached to form an unsubstituted or substituted N-containing heterocycloalkyl. In some embodiments, each R² is independently hydrogen, unsubstituted or substituted C₁-C₆ alkyl, unsubstituted or substituted C₃-C₆ cycloalkyl, or unsubstituted or substituted 3- to 6-membered heterocycloalkyl; or two R²² on the same N atom are taken together with the N atom to which they are attached to form an unsubstituted or substituted N-containing heterocycloalkyl. In some embodiments, each R²² is independently hydrogen, C₁-C₆ alkyl, C₃-C₆ cycloalkyl, or 3- to 6-membered heterocycloalkyl; or two R²² on the same N atom are taken together with the N atom to which they are attached to form an N-containing heterocycloalkyl. In some embodiments, each R²² is independently hydrogen, C₁-C₆ alkyl, or C₃-C₆ cycloalkyl; or two R²² on the same N atom are taken together with the N atom to which they are attached to form an N-containing heterocycloalkyl. In some embodiments, each R² is independently hydrogen or C₁-C₆ alkyl; or two R²² on the same N atom are taken together with the N atom to which they are attached to form an N-containing heterocycloalkyl.

In some embodiments, each R²³ is independently selected from hydrogen, C₁-C₆ alkyl, C₃-C₆ cycloalkyl, 3- to 6-membered heterocycloalkyl, phenyl, benzyl, 5-membered heteroaryl and 6-membered heteroaryl; or two R² groups are taken together with the N atom to which they are attached to form a 3- to 6-membered N-containing heterocycloalkyl. In some embodiments, each R² is independently selected from hydrogen, C₁-C₆ alkyl, C₃-C₆ cycloalkyl, and 3- to 6-membered heterocycloalkyl; or two R²³ groups are taken together with the N atom to which they are attached to form a 3- to 6-membered N-containing heterocycloalkyl. In some embodiments, each R²³ is independently selected from hydrogen and C₁-C₆ alkyl; or two R²³ groups are taken together with the N atom to which they are attached to form a 3- to 6-membered N-containing heterocycloalkyl.

In some embodiments, each R²⁴ is independently selected from C₁-C₆ alkyl, C₃-C₆ cycloalkyl, 3- to 6-membered heterocycloalkyl, phenyl, benzyl, 5-membered heteroaryl and 6-membered heteroaryl. In some embodiments, each R²⁴ is independently selected from C₁-C₆ alkyl, C₃-C₆ cycloalkyl, and 3- to 6-membered heterocycloalkyl. In some embodiments, each R²⁴ is independently selected from C₁-C₆ alkyl.

In some embodiments, described herein is a compound of Formula (I):

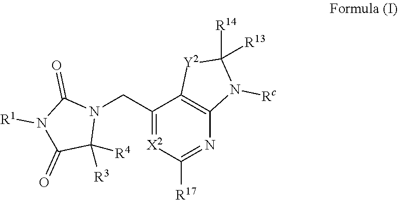

or a pharmaceutically acceptable salt or solvate thereof, wherein:
R¹ is unsubstituted or substituted phenyl, unsubstituted or substituted monocyclic heteroaryl, or unsubstituted or substituted bicyclic heteroaryl; wherein if R¹ is substituted, then it is substituted with R^{A} and 1-3 R⁵;
   R^{A} is unsubstituted or substituted C₁-C₆ alkyl, unsubstituted or substituted C₁-C₆ fluoroalkyl, unsubstituted or substituted phenyl, unsubstituted or substituted 5- or 6-membered heteroaryl, unsubstituted or substituted C₃-C₆ cycloalkyl, unsubstituted or substituted C₃-C₆ cycloalkenyl, unsubstituted or substituted 3- to 6-membered heterocycloalkyl, halogen, —CN, —SR^{A1}, —S(═O)R^{A1}, —S(═O)₂R^{A1}, —SF₅, or —SiMe₃;
   R^{A} is C₁-C₆ alkyl, C₁-C₆ fluoroalkyl, C₃-C₆ cycloalkyl, or 3- to 6-membered heterocycloalkyl;
or R^{A} is

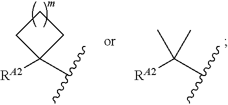

R^{A2} is halogen, —OH, —CN, C₁-C₄ alkyl, C₁-C₄ fluoroalkyl, C₁-C₄ alkoxy, C₁-C₄ fluoroalkoxyl, or C₁-C₄ hydroxyalkyl;
m is 0, 1, 2, or 3:
each R⁵ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl. —CN. —OH, —OR², —CO₂R²², —N(R²²)₂, —C(═O)N(R²²)₂, —NR²²C(═O)R²¹, —S(═O)₂N(R²²)₂, or —NR²²S(═O)₂R²¹;
or R¹ is

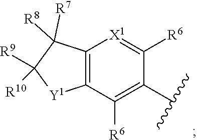

X is CR⁶ or N;
   each R⁶ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²¹, —CO₂R²², —N(R²²)₂, —C(═O)N(R²²)₂, —NR²²C(═O)R²¹, —S(═O)₂N(R²²)₂, or —NR²²S(═O)₂R²¹;
Y¹ is —O—, —NR¹¹—, or —SO₂—;
   R¹¹ is hydrogen, C₁-C₆ alkyl, C₁-C₆ fluoroalkyl, —C(═O)NH₂, —C(═O)R¹², or —S(═O)₂R¹²;
   R¹² is unsubstituted or substituted C₁-C₆ alkyl, unsubstituted or substituted C₃-C₆ cycloalkyl, or unsubstituted or substituted 3- to 6-membered heterocycloalkyl;
R⁷ and R⁸ are each independently hydrogen, —OH, C₁-C₆ alkyl, C₁-C₆ fluoroalkyl, or C₁-C₆ alkoxy;
or R⁷ and R⁸ are taken together with the carbon atom to which they are attached to form an unsubstituted or substituted C₃-C₆ cycloalkyl or unsubstituted or substituted 3- to 6-membered heterocycloalkyl;
R⁹ and R¹⁰ are each independently hydrogen, halogen, C₁-C₆ alkyl, C₁-C₆ fluoroalkyl, or C₁-C₆ alkoxy;
or R⁹ and R¹⁰ are taken together with the carbon atom to which they are attached to form a —C(═O)—.
or R⁹ and R¹⁰ are taken together with the carbon atom to which they are attached to form an unsubstituted or substituted C₃-C₆ cycloalkyl or unsubstituted or substituted 3- to 6-membered heterocycloalkyl;
R³ is hydrogen, unsubstituted or substituted C₁-C₆ alkyl, unsubstituted or substituted C₁-C₆ fluoroalkyl, unsubstituted or substituted C₁-C₆ alkoxy, unsubstituted or substituted phenyl, unsubstituted or substituted benzyl, unsubstituted or substituted monocyclic heteroaryl, unsubstituted or substituted C₃-C₆ cycloalkyl, or unsubstituted or substituted 3- to 6-membered heterocycloalkyl;
R⁴ is hydrogen or C₁-C₆ alkyl;
or R³ and R⁴ are taken together with the carbon atom to which they are attached to form a C₃-C₆ cycloalkyl or 3- to 6-membered heterocycloalkyl;
R^{c} is hydrogen, C₁-C₆ alkyl, C₁-C₆ fluoroalkyl, —C(═O)N(R²²)₂, —C(═O)R²¹, —C(═O)OR²², or —S(═O)₂R²¹;
X² is CR¹⁷ or N;
each R¹⁷ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²², —CO₂R²², —N(R²²)₂, —C(═O)N(R²², —OC(═O)R²¹, —NR²²C(═O)R²¹, —S(═O)₂N(R²²)₂, or —NR²²S(═O)₂R²¹;
R¹³ and R¹⁴ are each independently hydrogen, halogen, unsubstituted or substituted C₁-C₆ alkyl, C₁-C₆ fluoroalkyl, phenyl, monocyclic heteroaryl, C₃-C₆ cycloalkyl, 3- to 6-membered heterocycloalkyl, —CN, —OH, —OR²¹, —CO₂R²², —N(R²²)₂, —C(═O)N(R²²)₂, or —OC(═O)R²¹;
or R¹³ and R¹⁴ are taken together with the carbon atom to which they are attached to form a —C(═O)—;
or R¹³ and R¹⁴ are taken together with the carbon atom to which they are attached to form a C₃-C₆ cycloalkyl or 3- to 6-membered heterocycloalkyl;
Y² is —(CR¹⁵R¹⁶)ₙ—, —O—, or —O—CR¹⁵R¹⁶—;
   each R¹⁵ and R¹⁶ is independently hydrogen, halogen, cyano, C₁-C₆ alkyl, C₁-C₆ fluoroalkyl, C₁-C₆ alkoxy, phenyl, monocyclic heteroaryl, C₃-C₆ cycloalkyl, or 3- to 6-membered heterocycloalkyl;
   or R¹⁴ and one R¹⁶ on an adjacent carbon atom are taken together to form an alkene bond,
   n is 1 or 2;
each R²¹ is independently unsubstituted or substituted C₁-C₆ alkyl, unsubstituted or substituted C₃-C₆ cycloalkyl, unsubstituted or substituted 3- to 6-membered heterocycloalkyl, unsubstituted or substituted aryl, or unsubstituted or substituted heteroaryl;
each R²² is independently hydrogen, unsubstituted or substituted C₁-C₆ alkyl, unsubstituted or substituted C₃-C₆ cycloalkyl, unsubstituted or substituted 3- to 6-membered heterocycloalkyl, unsubstituted or substituted aryl, or unsubstituted or substituted heteroaryl; or two R²² on the same N atom are taken together with the N atom to which they are attached to form an unsubstituted or substituted N-containing heterocycloalkyl;
wherein each substituted alkyl, substituted fluoroalkyl, substituted aryl, substituted heteroaryl, substituted cycloalkyl, and substituted heterocycloalkyl is substituted with one or more R^{s} groups independently selected from the group consisting of halogen, C₁-C₆ alkyl, C₁-C₆ fluoroalkyl, C₃-C₆ cycloalkyl, 3- to 6-membered heterocycloalkyl, phenyl, benzyl, 5-membered heteroaryl, 6-membered heteroaryl. —CN, —OR²³, —CO₂R²³, —C(═O)N(R²³)₂, —N(R²³)₂, —NR²³C(═O)R²⁴, —SR²³, —S(═O)R²⁴, —SO₂R²⁴, or —SO₂N(R²³)₂;
   each R²³ is independently selected from hydrogen. C₁-C₆ alkyl, C₃-C₆ cycloalkyl, 3- to 6-membered heterocycloalkyl, phenyl, benzyl, 5-membered heteroaryl and 6-membered heteroaryl; or two R²³ groups are taken together with the N atom to which they are attached to form a 3- to 6-membered N-containing heterocycloalkyl;
   each R²⁴ is independently selected from C₁-C₆ alkyl, C₃-C₆ cycloalkyl, 3- to 6-membered heterocycloalkyl, phenyl, benzyl, 5-membered heteroaryl and 6-membered heteroaryl.

In some embodiments, described herein is a compound of Formula (I):

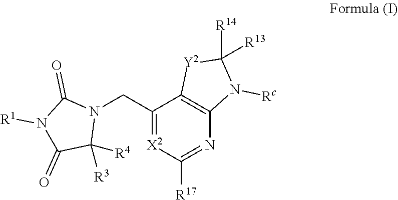

or a pharmaceutically acceptable salt or solvate thereof, wherein:
R¹ is unsubstituted or substituted phenyl, unsubstituted or substituted monocyclic heteroaryl, or unsubstituted or substituted bicyclic heteroaryl; wherein if R¹ is substituted, then it is substituted with R^{A} and 1-3 R⁵;
   R^{A} is unsubstituted or substituted C₁-C₆ alkyl, unsubstituted or substituted C₁-C₆ fluoroalkyl, unsubstituted or substituted phenyl, unsubstituted or substituted 5- or 6-membered heteroaryl, unsubstituted or substituted C₃-C₆ cycloalkyl, unsubstituted or substituted C₃-C₆ cycloalkenyl, unsubstituted or substituted 3- to 6-membered heterocycloalkyl, halogen, —CN, —SR^{A1}, —S(═O)R^{A1}, —S(═O)₂R^{A1}, —SF₅, or —SiMe₃;
   R^{A1} is C₁-C₆ alkyl, C₁-C₆ fluoroalkyl, C₃-C₆ cycloalkyl, or 3- to 6-membered heterocycloalkyl;
or R^{A} is

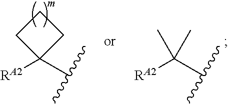

R^{A2} is halogen, —OH, —CN, C₁-C₄ alkyl, C₁-C₄ fluoroalkyl, C₁-C₄ alkoxy, C₁-C₄ fluoroalkoxyl, or C₁-C₄ hydroxyalkyl;
m is 0, 1, 2, or 3;
each R⁵ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²¹, —CO₂R²², —N(R²²)₂, —C(═O)N(R²²)₂, —NR²²C(═O)R²¹, —S(═O)₂N(R²², or —NR²²S(═O)₂R²¹;
or R¹ is

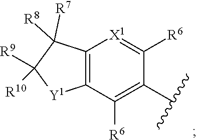

X¹ is CR⁶ or N;
   each R⁶ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²¹, —CO₂R²², —N(R²²)₂, —C(═O)N(R²²)₂, —NR²²C(═O)R²¹, —S(═O)₂N(R²²)₂, or —NR²²S(═O)₂R²¹;
Y¹ is —O—, —NR¹¹—, or —SO₂—;
   R¹¹ is hydrogen, C₁-C₆ alkyl, C₁-C₆ fluoroalkyl, —C(═O)NH₂, —C(═O)R¹², or —S(═O)₂R¹²;
   R¹² is unsubstituted or substituted C₁-C₆ alkyl, unsubstituted or substituted C₃-C₆ cycloalkyl, or unsubstituted or substituted 3- to 6-membered heterocycloalkyl;
R⁷ and R⁸ are each independently hydrogen, —OH, C₁-C₆ alkyl, C₁-C₆ fluoroalkyl, or C₁-C₆ alkoxy;
or R⁷ and R⁸ are taken together with the carbon atom to which they are attached to form an unsubstituted or substituted C₃-C₆ cycloalkyl or unsubstituted or substituted 3- to 6-membered heterocycloalkyl;
R⁹ and R¹⁰ are each independently hydrogen, halogen, C₁-C₆ alkyl, C₁-C₆ fluoroalkyl, or C₁-C₆ alkoxy;
or R⁹ and R¹⁰ are taken together with the carbon atom to which they are attached to form a —C(═O)—;
or R⁹ and R¹⁰ are taken together with the carbon atom to which they are attached to form an unsubstituted or substituted C₃-C₆ cycloalkyl or unsubstituted or substituted 3- to 6-membered heterocycloalkyl;
R³ is hydrogen, unsubstituted or substituted C₁-C₆ alkyl, unsubstituted or substituted C₁-C₆ fluoroalkyl, unsubstituted or substituted C₁-C₆ alkoxy, unsubstituted or substituted phenyl, unsubstituted or substituted benzyl, unsubstituted or substituted monocyclic heteroaryl, unsubstituted or substituted C₃-C₆ cycloalkyl, or unsubstituted or substituted 3- to 6-membered heterocycloalkyl;
R⁴ is hydrogen or C₁-C₆ alkyl;
or R³ and R⁴ are taken together with the carbon atom to which they are attached to form a C₃-C₆ cycloalkyl or 3- to 6-membered heterocycloalkyl;
R^{c} is hydrogen, C₁-C₆ alkyl, C₁-C₆ fluoroalkyl, —C(═O)N(R²²)₂, —C(═O)R²¹, —C(═O)OR²², or —S(═O)₂R²¹;
X² is CR¹⁷ or N;
each R¹⁷ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²¹, —CO₂R²², —N(R²²)₂, —C(═O)N(R²²)₂, —OC(═O)R²¹, —NR²²C(═O)R²¹, —S(═O)₂N(R²²)₂, or —NR²²S(═O)₂R²¹;
R¹³ and R¹⁴ are each independently hydrogen, halogen, unsubstituted or substituted C₁-C₆ alkyl, C₁-C₆ fluoroalkyl, phenyl, monocyclic heteroaryl, C₃-C₆ cycloalkyl, 3- to 6-membered heterocycloalkyl, —CN, —OH, —OR²¹, —CO₂R²², —N(R²²)₂, —C(═O)N(R²²)₂, or —OC(═O)R²¹;
or R¹³ and R¹⁴ are taken together with the carbon atom to which they are attached to form a —C(═O)—;
or R¹³ and R¹⁴ are taken together with the carbon atom to which they are attached to form a C₃-C₆ cycloalkyl or 3- to 6-membered heterocycloalkyl;
Y² is —(CR¹⁵R¹⁶)ₙ—, —O—, or —O—CR¹⁵R¹⁶—;
   each R¹⁵ and R¹⁶ is independently hydrogen, halogen, cyano, C₁-C₆ alkyl, C₁-C₆ fluoroalkyl, C₁-C₆ alkoxy, phenyl, monocyclic heteroaryl, C₃-C₆ cycloalkyl, or 3- to 6-membered heterocycloalkyl;
   or R¹⁴ and one R¹⁶ on an adjacent carbon atom are taken together to form an alkene bond;
   n is 1 or 2:
each R²¹ is independently unsubstituted or substituted C₁-C₆ alkyl, unsubstituted or substituted C₃-C₆ cycloalkyl, unsubstituted or substituted 3- to 6-membered heterocycloalkyl, unsubstituted or substituted aryl, or unsubstituted or substituted heteroaryl;
each R²² is independently hydrogen, unsubstituted or substituted C₁-C₆ alkyl, unsubstituted or substituted C₃-C₆ cycloalkyl, unsubstituted or substituted 3- to 6-membered heterocycloalkyl, unsubstituted or substituted aryl, or unsubstituted or substituted heteroaryl; or two R²² on the same N atom are taken together with the N atom to which they are attached to form an unsubstituted or substituted N-containing heterocycloalkyl;
wherein each substituted alkyl, substituted fluoroalkyl, substituted aryl, substituted heteroaryl, substituted cycloalkyl, and substituted heterocycloalkyl is substituted with one or more R^{s} groups independently selected from the group consisting of halogen, C₁-C₆ alkyl, C₁-C₆ fluoroalkyl, C₃-C₆ cycloalkyl, 3- to 6-membered heterocycloalkyl, phenyl, benzyl, 5-membered heteroaryl, 6-membered heteroaryl, —CN, —OR²³, —CO₂R²³—C(═O)N(R²³)₂, —N(R²³)₂, —NR²³C(═O)R²³, —SR²³, —S(═O)R²⁴, —SO₂R²⁴, or —SO₂N(R²³)₂;
   each R²³ is independently selected from hydrogen, C₁-C₆ alkyl, C₃-C₆ cycloalkyl, 3- to 6-membered heterocycloalkyl, phenyl, benzyl, 5-membered heteroaryl and 6-membered heteroaryl; or two R² groups are taken together with the N atom to which they are attached to form a 3- to 6-membered N-containing heterocycloalkyl; and
   each R²⁴ is independently selected from C₁-C₆ alkyl, C₃-C₆ cycloalkyl, 3- to 6-membered heterocycloalkyl, phenyl, benzyl, 5-membered heteroaryl and 6-membered heteroaryl,
   provided that when Y² is —O—CR¹⁵R¹⁶, then R¹³ and R¹⁴ cannot be taken together with the carbon atom to which they are attached to form a —C(═O).

In some embodiments, R is hydrogen, C₁-C₆ alkyl, C₁-C₆ fluoroalkyl, —C(═O)N(R²²)₂, —C(═O)R²¹, —C(═)OR²², or —S(═O)₂R²¹. In some embodiments, R is hydrogen, C₁-C₆ alkyl, C₁-C₆ fluoroalkyl, —C(═O)N(R²²)₂, —C(═O)R²¹, or —C(═O)OR²². In some embodiments, R^{c} is hydrogen, C₁-C₆ alkyl, C₁-C₆ fluoroalkyl, or —C(═O)OR²². In some embodiments, R^{c} is hydrogen, C₁-C₆ alkyl, or —C(═O)OR²². In some embodiments, R^{c} is hydrogen, C₁-C₆ alkyl, or C₁-C₆ fluoroalkyl. In some embodiments, R^{c} is hydrogen or C₁-C₆ alkyl. In some embodiments, R^{c} is hydrogen.

In some embodiments, X² is CR¹⁷ or N. In some embodiments, X² is CR¹⁷. In some embodiments, X² is N.

In some embodiments, Y² is —(CR¹⁵R¹⁶)₆—, —O—, or —O—CR¹⁵R¹⁶—. In some embodiments, Y² is —(CR¹⁵R¹⁶)ₙ—, and n is 1 or 2. In some embodiments, Y² is —(CR¹⁵R¹⁶)— or —(CR¹⁵R¹⁶)—(CR¹⁵R¹⁶)—. In some embodiments, Y² is —O—. In some embodiments, Y² is —O—CR¹⁵R¹⁶—.

In some embodiments, R¹³ and R¹⁴ are each independently hydrogen, halogen, unsubstituted or substituted C₁-C₆ alkyl, C₁-C₆ fluoroalkyl, phenyl, monocyclic heteroaryl, C₃-C₆ cycloalkyl, 3- to 6-membered heterocycloalkyl, —CN, —OH, —OR²¹, —CO₂R²², —N(R²)₂, —C(═O)N(R²²)₂, or —OC(═O)R²¹. In some embodiments, R¹³ and R¹⁴ are each independently hydrogen, halogen, C₁-C₆ alkyl, C₁-C₆ fluoroalkyl, C₃-C₆ cycloalkyl, or 3- to 6-membered heterocycloalkyl. In some embodiments, R¹³ and R¹⁴ are each independently hydrogen or C₁-C₆ alkyl. In some embodiments, R¹³ and R¹⁴ are each hydrogen.

In some embodiments, R¹³ and R¹⁴ are taken together with the carbon atom to which they are attached to form a —C(═O)—.

In some embodiments, R¹³ and R¹⁴ are taken together with the carbon atom to which they are attached to form a C₃-C₆ cycloalkyl or 3- to 6-membered heterocycloalkyl. In some embodiments, R¹³ and R¹⁴ are taken together with the carbon atom to which they are attached to form a C₃-C₆ cycloalkyl. In some embodiments, R¹³ and R¹⁴ are taken together with the carbon atom to which they are attached to form a C₃-C₄ cycloalkyl. In some embodiments. R¹³ and R¹⁴ are taken together with the carbon atom to which they are attached to form a 3- to 6-membered heterocycloalkyl. In some embodiments, R¹³ and R⁴ are taken together with the carbon atom to which they are attached to form a 3- or 4-membered heterocycloalkyl.

In some embodiments, each R¹⁵ and R¹⁶ is independently hydrogen, halogen, cyano, C₁-C₆ alkyl, C₁-C₆ fluoroalkyl, C₁-C₆ alkoxy, phenyl, monocyclic heteroaryl, C₃-C₆ cycloalkyl, or 3- to 6-membered heterocycloalkyl. In some embodiments, each R¹⁵ and R¹⁶ is independently hydrogen, halogen, C₁-C₆ alkyl, C₁-C₆ fluoroalkyl, or C₃-C₆ cycloalkyl. In some embodiments, each R¹⁵ and R¹⁶ is independently hydrogen, C₁-C₆ alkyl, C₁-C₆ fluoroalkyl, or C₃-C₆ cycloalkyl. In some embodiments, each R¹⁵ and R¹⁶ is independently hydrogen or C₁-C₆ alkyl. In some embodiments, each R¹⁵ and R¹⁶ is independently hydrogen.

In some embodiments, R¹⁵ and one R¹⁶ on an adjacent carbon atom are taken together to form an alkene bond.

In some embodiments, each R⁷ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²¹, —CO₂R²², —N(R²²)₂, —C(═O)N(R²²)₂, —OC(═O)R²¹, —NR²²C(═O)R²¹, —S(═O)₂N(R²²)₂, or —NR²²S(═O)₂R²¹. In some embodiments, each R¹⁷ is independently hydrogen, halogen, unsubstituted or substituted C₁-C₆ alkyl, unsubstituted or substituted C₁-C₆ fluoroalkyl, —CN, —OH, —OR²¹—CO₂R²², —N(R²²)₂, —C(═O)N(R²²)₂, —OC(═O)R²¹, —NR²²C(═O)R²¹, —S(═O)₂N(R²²)₂, or —NR²²S(═O)₂R²¹. In some embodiments, each R¹⁷ is independently hydrogen, halogen, C₁-C₆ alkyl, C₁-C₆ fluoroalkyl, —CN, —OH, —OR²¹, —N(R²²)₂, or —C(═O)N(R′)₂. In some embodiments, each R¹⁷ is independently hydrogen, halogen, C₁-C₆ alkyl, or C₁-C₆ fluoroalkyl. In some embodiments, each R¹⁷ is hydrogen.

In some embodiments, R^{c} is hydrogen, C₁-C₆ alkyl, or —C(═O)OR²; X² is CR¹⁷; each R¹⁷ is independently hydrogen, halogen, C₁-C₆ alkyl, or C₁-C₆ fluoroalkyl; R¹³ and R¹⁴ are each independently hydrogen, halogen, C₁-C₆ alkyl, C₁-C₆ fluoroalkyl, C₃-C₆ cycloalkyl, or 3- to 6-membered heterocycloalkyl; or R¹³ and R¹⁴ are taken together with the carbon atom to which they are attached to form a —C(═O)—; or R¹³ and R¹⁴ are taken together with the carbon atom to which they are attached to form a C₃-C₄ cycloalkyl; Y² is —(CR¹⁵R¹⁶)ₙ—, —O—, or —O—CR¹⁵R¹⁶—; and each R¹⁵ and R¹⁶ is independently hydrogen, halogen, C₁-C₆ alkyl, C₁-C₆ fluoroalkyl, or C₃-C₆ cycloalkyl, or R¹⁴ and one R¹⁶ on an adjacent carbon atom are taken together to form an alkene bond.

In some embodiments, R^{c} is hydrogen; X² is CR¹⁷; each R⁷ is hydrogen; R¹³ and R¹⁴ are each hydrogen; or R¹³ and R¹⁴ are taken together with the carbon atom to which they are attached to form a —C(═O)—; and Y² is —(CR¹⁵R¹⁶)ₙ—, —O—, or —O—CR¹⁵R¹⁶—. In some embodiments, each R¹⁵ and R¹⁶ is independently hydrogen, C₁-C₆ alkyl, C₁-C₆ fluoroalkyl, or C₃-C₆ cycloalkyl.

In some embodiments,

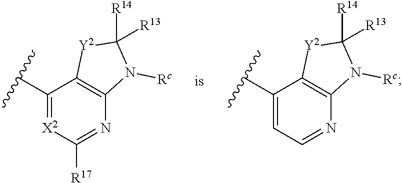

and Y² is —CR¹⁵R¹⁶- or —CR¹⁵R¹⁶—CR¹⁵R¹⁶—.

In some embodiments,

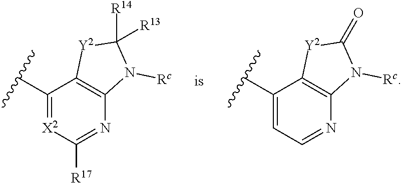

In some embodiments, R¹⁴ and one R¹⁶ on an adjacent carbon atom are taken together to form an alkene bond.

In some embodiments, R³ is hydrogen, unsubstituted or substituted C₁-C₆ alkyl, unsubstituted or substituted C₁-C₆ fluoroalkyl, unsubstituted or substituted C₁-C₆ alkoxy, unsubstituted or substituted phenyl, unsubstituted or substituted benzyl, unsubstituted or substituted monocyclic heteroaryl, unsubstituted or substituted C₃-C₆ cycloalkyl, or unsubstituted or substituted 3- to 6-membered heterocycloalkyl. In some embodiments, R³ is hydrogen, unsubstituted or substituted C₁-C₆ alkyl, unsubstituted or substituted C₁-C₆ fluoroalkyl, unsubstituted or substituted phenyl, unsubstituted or substituted benzyl, or unsubstituted or substituted C₃-C₆ cycloalkyl. In some embodiments, R³ is unsubstituted or substituted C₁-C₆ alkyl, phenyl, benzyl, or C₃-C₆ cycloalkyl. In some embodiments, R³ is C₁-C₆ alkyl which is unsubstituted or substituted by —OH, phenyl, benzyl, or C₃-C₆ cycloalkyl. In some embodiments, R³ is methyl, ethyl, propyl, isopropyl, butyl, isobutyl, sec-butyl, phenyl, benzyl, cyclopropyl, cyclobutyl, cyclopentyl, cyclohexyl, or —CH₂OH

In some embodiments, R⁴ is hydrogen or C₁-C₆ alkyl. In some embodiments, R⁴ is hydrogen or C₁-C₄ alkyl. In some embodiments, R⁴ is hydrogen or methyl.

In some embodiments, R³ and R⁴ are taken together with the carbon atom to which they are attached to form a C₃-C₆ cycloalkyl or 3- to 6-membered heterocycloalkyl. In some embodiments, R³ and R⁴ are taken together with the carbon atom to which they are attached to form a C₃-C₆ cycloalkyl. In some embodiments, R³ and R⁴ are taken together with the carbon atom to which they are attached to form a cyclopropyl, cyclobutyl, cyclopentyl, or cyclohexyl.

In some embodiments, R³ is C₁-C₆ alkyl; and R⁴ is C₁-C₆ alkyl; or R³ and R⁴ are taken together with the carbon atom to which they are attached to form a C₃-C₆ cycloalkyl.

In some embodiments, R¹ is unsubstituted or substituted phenyl or unsubstituted or substituted monocyclic heteroaryl; wherein if R¹ is substituted, then it is substituted with R^{A} and 1-3 R⁵.

In some embodiments, each R⁵ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²¹, —CO₂R²², —N(R²²)₂, —C(═O)N(R²²)₂—NR²²C(═O)R²¹, —S(═O)₂N(R²²)₂, or —NR²²S(═O)₂R²¹. In some embodiments, each R⁵ is independently hydrogen, halogen, unsubstituted or substituted C₁-C₆ alkyl, unsubstituted or substituted C₁-C₆ fluoroalkyl, unsubstituted or substituted C₃-C₆ cycloalkyl, unsubstituted or substituted 3- to 6-membered heterocycloalkyl. —CN. —OH, —OR²¹, —N(R²²)₂, or —C(═O)N(R²²)₂. In some embodiments, each R⁵ is independently hydrogen, halogen, C₁-C₆ alkyl, C₁-C₆ fluoroalkyl, C₃-C₆ cycloalkyl, 3- to 6-membered heterocycloalkyl, —CN, or —OR²¹. In some embodiments, each R⁵ is independently hydrogen or C₁-C₆ alkyl. In some embodiments, each R⁵ is hydrogen.

In some embodiments, R¹ is

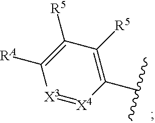

wherein X³ and X⁴ are each independently CR⁵ or N; and each R⁵ is independently hydrogen, halogen, C₁-C₆ alkyl, C₁-C₆ fluoroalkyl, C₃-C₆ cycloalkyl, 3- to 6-membered heterocycloalkyl, —CN, or —OR²¹. In some embodiments, X³ and X⁴ are each CR⁵; and each R⁵ is hydrogen.

In some embodiments, R^{A} is C₁-C₆ alkyl, C₁-C₆ fluoroalkyl, halogen, —CN, —SR^{A1}, —S(═O)R^{A1}, —S(═O)₂R^{A1}, —SF₅, or —SiMe₃; R^{A} is C₁-C₄ alkyl, or C₁-C₄ fluoroalkyl; or R^{A} is

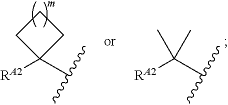

R^{A2} is halogen, —CN, C₁-C₄ alkyl, or C₁-C₄ fluoroalkyl; and m is 0 or 1. In some embodiments, R^{A} is —CH₃, —CH₂CH₃, —CH(CH₃)₂, —C(CH₃)₃, —CF₃, —F, —Cl, —Br, —CN, —SCF₃, —S(═O)CF₃, —S(═O)₂CF₃, —S(═O)₂CHF₂, —S(═O)₂C(CH₃)₃, —S(═O)₂-(cyclopropyl), —SF₅, or —SiMe₃; or R^{A} is

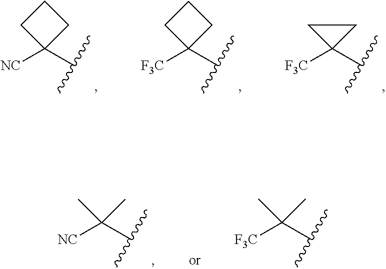

In some embodiments, R¹ is

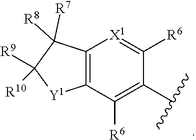

In some embodiments, X¹ is CR⁶ or N. In some embodiments, X¹ is CR⁶. In some embodiments, X¹ is N.

In some embodiments, Y¹ is —O—, —NR¹¹—, or —SO₂—. In some embodiments, Y¹ is —O— or —NR¹¹—. In some embodiments, Y¹ is —O—. In some embodiments, Y¹ is —NR¹¹—. In some embodiments, Y¹ is —SO₂—.

In some embodiments, R¹¹ is hydrogen, C₁-C₆ alkyl, C₁-C₆ fluoroalkyl, —C(═O)NH₂, —C(═O)R¹¹, or —S(═O)₂R¹². In some embodiments, R¹¹ is hydrogen or C₁-C₆ alkyl. In some embodiments, R¹¹ is hydrogen. In some embodiments, R¹¹ is —C(═O)NH₂. —C(═O)R¹², or —S(═O)₂R¹².

In some embodiments, R¹² is unsubstituted or substituted C₁-C₆ alkyl, unsubstituted or substituted C₃-C₆ cycloalkyl, or unsubstituted or substituted 3- to 6-membered heterocycloalkyl. In some embodiments, R¹² is unsubstituted or substituted C₁-C₆ alkyl or unsubstituted or substituted C₃-C₆ cycloalkyl. In some embodiments, R¹² is C₁-C₆ alkyl or C₃-C₆ cycloalkyl. In some embodiments, R¹² is C₁-C₆ alkyl. In some embodiments. R¹² is C₃-C₆ cycloalkyl.

In some embodiments, each R⁶ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH. —OR²¹, —CO₂R²², —N(R²²)₂, —C(═O)N(R²²)₂, —NR²²C(═O)R²¹. —S(═O)₂N(Ru)₂, or —NR²²S(═O)₂R²¹. In some embodiments, each R⁶ is independently hydrogen, halogen, C₁-C₆ alkyl, C₁-C₆ fluoroalkyl, unsubstituted or substituted C₃-C₆ cycloalkyl, unsubstituted or substituted 3- to 6-membered heterocycloalkyl, —CN, —OH. —OR²¹, —CO₂R². —N(R)₂, —C(═O)N(R²²)₂, —NR²²C(═O)R²¹, —S(═O)₂N(R²²)₂, or —NR²²S(═O)₂R²¹. In some embodiments, each R⁶ is independently hydrogen, halogen, C₁-C₆ alkyl, C₁-C₆ fluoroalkyl, —CN, —OH, —OR²¹, —N(R²²)₂, or —C(═O)N(R²²)₂. In some embodiments, each R⁶ is independently hydrogen, halogen, or C₁-C₆ alkyl. In some embodiments, each R⁶ is hydrogen.

In some embodiments, R⁷ and R⁸ are each independently hydrogen, —OH, C₁-C₆ alkyl, C₁-C₆ fluoroalkyl, or C₁-C₆ alkoxy. In some embodiments, R⁷ and R⁸ are each independently hydrogen, —OH, or C₁-C₆ alkyl. In some embodiments, R⁷ and R⁸ are each independently hydrogen or C₁-C₆ alkyl. In some embodiments, R⁷ and R⁸ are each independently hydrogen or methyl.

In some embodiments, R⁷ and R⁸ are taken together with the carbon atom to which they are attached to form an unsubstituted or substituted C₃-C₆ cycloalkyl or unsubstituted or substituted 3- to 6-membered heterocycloalkyl. In some embodiments, R⁷ and R⁸ are taken together with the carbon atom to which they are attached to form an unsubstituted or substituted C₃-C₆ cycloalkyl. In some embodiments, R⁷ and R⁸ are taken together with the carbon atom to which they are attached to form an unsubstituted or substituted C₃-C₄ cycloalkyl. In some embodiments, R⁷ and R⁸ are taken together with the carbon atom to which they are attached to form a 3- to 6-membered heterocycloalkyl. In some embodiments, R⁷ and R⁸ are taken together with the carbon atom to which they are attached to form a 3- or 4-membered heterocycloalkyl.

In some embodiments, R⁹ and R¹⁰ are each independently hydrogen, halogen, C₁-C₆ alkyl, C₁-C₆ fluoroalkyl, or C₁-C₆ alkoxy. In some embodiments, R⁹ and R¹⁰ are each independently hydrogen, halogen, C₁-C₆ alkyl, or C₁-C₆ fluoroalkyl. In some embodiments, R⁹ and R¹⁰ are each independently hydrogen or C₁-C₆ alkyl. In some embodiments, R⁹ and R¹⁰ are each independently hydrogen or methyl.

In some embodiments, R⁹ and R¹⁰ are taken together with the carbon atom to which they are attached to form a —C(═O)—.

In some embodiments, R⁹ and R¹⁰ are taken together with the carbon atom to which they are attached to form an unsubstituted or substituted C₃-C₆ cycloalkyl or unsubstituted or substituted 3- to 6-membered heterocycloalkyl. In some embodiments, R⁹ and R¹⁰ are taken together with the carbon atom to which they are attached to form an unsubstituted or substituted C₃-C₆ cycloalkyl. In some embodiments, R⁹ and R¹⁰ are taken together with the carbon atom to which they are attached to form an unsubstituted or substituted C₃-C₄ cycloalkyl. In some embodiments, R⁹ and R¹⁰ are taken together with the carbon atom to which they are attached to form a 3- to 6-membered heterocycloalkyl. In some embodiments, R⁹ and R¹⁰ are taken together with the carbon atom to which they are attached to form a 3- or 4-membered heterocycloalkyl.

In some embodiments, R¹ is

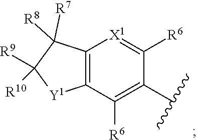

X¹ is CR⁶ or N; and each R⁶ is independently hydrogen, halogen, C₁-C₆ alkyl, C₁-C₆ fluoroalkyl, unsubstituted or substituted C₃-C₆ cycloalkyl, unsubstituted or substituted 3- to 6-membered heterocycloalkyl, —CN, —OH, —OR²¹, —CO₂R²², —N(R²²)₂, —C(═O)N(R²²)₂, —NR²²C(═O)R²¹, —S(═O)₂N(R²²)₂, or —NR²²S(═O)₂R²¹.

In some embodiments, Y¹ is —NR¹¹—; R¹¹ is hydrogen, C₁-C₆ alkyl, C₁-C₆ fluoroalkyl, —C(═O)NH₂, —C(═O)R¹², or —S(═O)R¹²; and R¹² is unsubstituted or substituted C₁-C₆ alkyl or unsubstituted or substituted C₃-C₆ cycloalkyl.

In some embodiments, R⁷ and R⁸ are each independently hydrogen, —OH, C₁-C₆ alkyl, C₁-C₆ fluoroalkyl, or C₁-C₆ alkoxy; or R⁷ and R⁸ are taken together with the carbon atom to which they are attached to form an unsubstituted or substituted C₃-C₄ cycloalkyl; R⁹ and R¹⁰ are each independently hydrogen, halogen, C₁-C₆ alkyl, C₁-C₆ fluoroalkyl, or C₁-C₆ alkoxy; or R⁹ and R¹⁰ are taken together with the carbon atom to which they are attached to form a —C(═O)—; or R⁹ and R¹⁰ are taken together with the carbon atom to which they are attached to form an unsubstituted or substituted C₃-C₄ cycloalkyl.

In some embodiments, described herein is a compound of Formula (II):

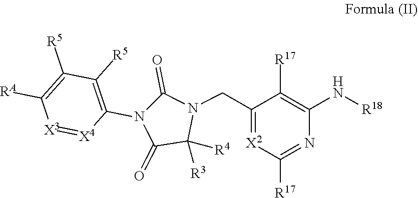

or a pharmaceutically acceptable salt or solvate thereof, wherein:
R^{A} is unsubstituted or substituted C₁-C₆ fluoroalkyl, unsubstituted or substituted phenyl, unsubstituted or substituted 5- or 6-membered heteroaryl, unsubstituted or substituted C₃-C₆ cycloalkyl, unsubstituted or substituted C₃-C₆ cycloalkenyl, or unsubstituted or substituted 3- to 6-membered heterocycloalkyl, halogen, —CN, —SF₅, or —SiMe₃;
or R^{A} is

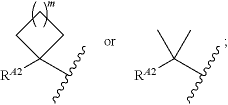

R^{A2} is halogen, —OH, —CN, C₁-C₄ alkyl, C₁-C₄ fluoroalkyl, C₁-C₄ alkoxy, C₁-C₄ fluoroalkoxyl, or C₁-C₄ hydroxyalkyl;
m is 0, 1, 2, or 3;
X³ and X⁴ are each independently CR⁵ or N;
R³ is hydrogen, unsubstituted or substituted C₁-C₆ alkyl, unsubstituted or substituted C₁-C₆ fluoroalkyl, unsubstituted or substituted C₁-C₆ alkoxy, unsubstituted or substituted phenyl, unsubstituted or substituted benzyl, unsubstituted or substituted monocyclic heteroaryl, unsubstituted or substituted C₃-C₆ cycloalkyl, or unsubstituted or substituted 3- to 6-membered heterocycloalkyl;
R⁴ is hydrogen or C₁-C₆ alkyl;
or R³ and R⁴ are taken together with the carbon atom to which they are attached to form a C₃-C₆ cycloalkyl or 3- to 6-membered heterocycloalkyl;
each R⁵ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²¹, —CO₂R²², —N(R²²)₂, —C(═O)N(R²²)₂, —NR—C(═O)R²¹, —S(═O)₂N(R²²)₂, or —NR²²S(═O)₂R²¹;
X² is CR¹⁷ or N;
each R¹⁷ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²¹, —CO₂R²², —N(R²²)₂, —C(═O)N(R²²)₂, —NR²²C(═O)R²¹, —S(═O)₂N(R²²)₂, or —NR²²S(═O)₂R²¹,
R¹⁸ is hydrogen, unsubstituted or substituted C₁-C₆ alkyl, unsubstituted or substituted C₁-C₆ fluoroalkyl, unsubstituted or substituted C₁-C₆ heteroalkyl, unsubstituted or substituted C₁-C₆ alkenyl, unsubstituted or substituted C₁-C₆ alkynyl, unsubstituted or substituted carbocycle, unsubstituted or substituted heterocycle, —CHR²-(unsubstituted or substituted carbocycle), —CHR²⁰-(unsubstituted or substituted heterocycle), —C(═O)R¹⁹, —CO₂R¹⁹, or —C(═O)NHR¹⁹;
   R¹⁹ is hydrogen, unsubstituted or substituted C₁-C₆ alkyl, unsubstituted or substituted C₃-C₁₀ cycloalkyl, unsubstituted or substituted 3- to 10-membered heterocycloalkyl;
   R²⁰ is hydrogen or —CH₃:
each R²¹ is independently unsubstituted or substituted C₁-C₆ alkyl, unsubstituted or substituted C₃-C₆ cycloalkyl, unsubstituted or substituted 3- to 6-membered heterocycloalkyl, unsubstituted or substituted aryl, or unsubstituted or substituted heteroaryl;
each R²² is independently hydrogen, unsubstituted or substituted C₁-C₆ alkyl, unsubstituted or substituted C₃-C₆ cycloalkyl, unsubstituted or substituted 3- to 6-membered heterocycloalkyl, unsubstituted or substituted aryl, or unsubstituted or substituted heteroaryl; or two R² on the same N atom are taken together with the N atom to which they are attached to form an unsubstituted or substituted N-containing heterocycloalkyl;
wherein each substituted alkyl, substituted fluoroalkyl, substituted aryl, substituted heteroaryl, substituted cycloalkyl, and substituted heterocycloalkyl is substituted with one or more R^{s} groups independently selected from the group consisting of halogen, C₁-C₆ alkyl, C₁-C₆ fluoroalkyl, C₃-C₆ cycloalkyl, 3- to 6-membered heterocycloalkyl, phenyl, benzyl, 5-membered heteroaryl, 6-membered heteroaryl, —CN, —OR²³, —CO₂R²³, —C(═O)N(R²³)₂, —N(R²³)₂, —NR²³C(═O)R²⁴, —SR²³, —S(═O)R²⁴, —SO₂R²⁴, or —SO₂N(R²³)₂;
   each R²³ is independently selected from hydrogen, C₁-C₆ alkyl, C₃-C₆ cycloalkyl, 3- to 6-membered heterocycloalkyl, phenyl, benzyl, 5-membered heteroaryl and 6-membered heteroaryl; or two R²³ groups are taken together with the N atom to which they are attached to form a 3- to 6-membered N-containing heterocycloalkyl:
each R²⁴ is independently selected from C₁-C₆ alkyl, C₃-C₆ cycloalkyl, 3- to 6-membered heterocycloalkyl, phenyl, benzyl, 5-membered heteroaryl and 6-membered heteroaryl.

### Additional Embodiments of Formula (II)

In some embodiments, R³ is hydrogen, unsubstituted or substituted C₁-C₆ alkyl, unsubstituted or substituted C₁-C₆ fluoroalkyl, unsubstituted or substituted C₁-C₆ alkoxy, unsubstituted or substituted phenyl, unsubstituted or substituted benzyl, unsubstituted or substituted monocyclic heteroaryl, unsubstituted or substituted C₃-C₆ cycloalkyl, or unsubstituted or substituted 3- to 6-membered heterocycloalkyl. In some embodiments, R³ is hydrogen, unsubstituted or substituted C₁-C₆ alkyl, unsubstituted or substituted C₁-C₆ fluoroalkyl, unsubstituted or substituted phenyl, unsubstituted or substituted benzyl, or unsubstituted or substituted C₃-C₆ cycloalkyl. In some embodiments, R³ is unsubstituted or substituted C₁-C₆ alkyl, phenyl, benzyl, or C₃-C₆ cycloalkyl. In some embodiments, R³ is C₁-C₆ alkyl which is unsubstituted or substituted by —OH, phenyl, benzyl, or C₃-C₆ cycloalkyl. In some embodiments, R³ is methyl, ethyl, propyl, isopropyl, butyl, isobutyl, sec-butyl, phenyl, benzyl, cyclopropyl, cyclobutyl, cyclopentyl, cyclohexyl, or —CH₂OH

In some embodiments, R⁴ is hydrogen or C₁-C₆ alkyl. In some embodiments, R⁴ is hydrogen or C₁-C₄ alkyl. In some embodiments, R⁴ is hydrogen or methyl.

In some embodiments, R³ and R⁴ are taken together with the carbon atom to which they are attached to form a C₃-C₆ cycloalkyl or 3- to 6-membered heterocycloalkyl. In some embodiments, R³ and R⁴ are taken together with the carbon atom to which they are attached to form a C₃-C₆ cycloalkyl. In some embodiments, R³ and R⁴ are taken together with the carbon atom to which they are attached to form a cyclopropyl, cyclobutyl, cyclopentyl, or cyclohexyl.

In some embodiments, R³ is C₁-C₆ alkyl; and R⁴ is C₁-C₆ alkyl; or R³ and R⁴ are taken together with the carbon atom to which they are attached to form a C₃-C₆ cycloalkyl.

In some embodiments, each R⁵ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²², —CO₂R²², —N(R²²)₂, —C(═O)N(R²²)₂, —NR²²C(═O)R²², —S(═O)₂N(R²²)₂, or —NR²²S(═O)₂R²¹. In some embodiments, each R⁵ is independently hydrogen, halogen, unsubstituted or substituted C₁-C₆ alkyl, unsubstituted or substituted C₁-C₆ fluoroalkyl, unsubstituted or substituted C₃-C₆ cycloalkyl, unsubstituted or substituted 3- to 6-membered heterocycloalkyl, —CN, —OH, —OR²¹, —N(R²)₂, or —C(═O)N(R²²)₂. In some embodiments, each R⁵ is independently hydrogen, halogen, C₁-C₆ alkyl, C₁-C₆ fluoroalkyl, C₃-C₆ cycloalkyl, 3- to 6-membered heterocycloalkyl, —CN, or —OR²¹. In some embodiments, each R⁵ is independently hydrogen or C₁-C₆ alkyl. In some embodiments, each R⁵ is hydrogen.

In some embodiments, each R⁵ is independently hydrogen, halogen, C₁-C₆ alkyl, C₁-C₆ fluoroalkyl, C₃-C₆ cycloalkyl, 3- to 6-membered heterocycloalkyl, —CN, or —OR²¹.

In some embodiments, X³ and X⁴ are each CR⁵; and each R⁵ is hydrogen.

In some embodiments, R^{A} is C₁-C₄ fluoroalkyl, halogen, —CN, —SF₅, or —SiMe₃; or R^{A} is

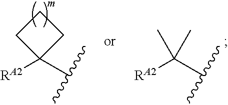

R^{A2} is halogen, —CN, C₁-C₄ alkyl, or C₁-C₄ fluoroalkyl; and m is 1.

In some embodiments, R^{A} is —CF₃, —F, —Cl, —Br, —CN, —SCF₃, —SF₅, or —SiMe₃; or R^{A} is

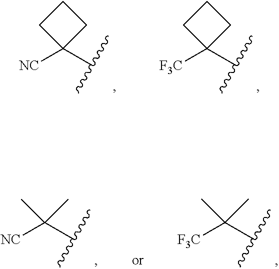

In some embodiments, X² is CR¹⁷ or N. In some embodiments, X² is CR¹⁷. In some embodiments, X² is N.

In some embodiments, each R¹⁷ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²¹, —CO₂R²¹, —N(R²²)₂, —C(═O)N(R²²)₂, —OC(═O)R²¹, —NR²²C(═O)R²¹, —S(═O)₂N(R²²)₂, or —NR²²S(═O)₂R²¹. In some embodiments, each R¹⁷ is independently hydrogen, halogen, unsubstituted or substituted C₁-C₆ alkyl, unsubstituted or substituted C₁-C₆ fluoroalkyl, —CN, —OH, —OR²¹, —CO₂R²², —N(R²²)₂, —C(═O)N(R²²)₂, —OC(═O)R²¹, —NR²C(═O)R²¹, —S(═O)₂N(R²²)₂, or —NR²²S(═O)₂R²¹. In some embodiments, each R¹⁷ is independently hydrogen, halogen, C₁-C₆ alkyl, C₁-C₆ fluoroalkyl, —CN, —OH. —OR²¹. —N(R²²)₂, or —C(═O)N(R²²)₂. In some embodiments, each R¹⁷ is independently hydrogen, halogen, C₁-C₆ alkyl, or C₁-C₆ fluoroalkyl. In some embodiments, each R¹⁷ is hydrogen.

In some embodiments, X² is CR¹⁷; and each R¹⁷ is independently hydrogen, halogen, C₁-C₆ alkyl, or C₁-C₆ fluoroalkyl.

In some embodiments, X² is CR¹⁷; and each R¹⁷ is hydrogen.

In some embodiments, R¹⁸ is hydrogen, unsubstituted or substituted C₁-C₆ alkyl, unsubstituted or substituted C₁-C₆ fluoroalkyl, unsubstituted or substituted C₁-C₆ heteroalkyl, unsubstituted or substituted C₁-C₆ alkenyl, unsubstituted or substituted C₁-C₆ alkynyl, unsubstituted or substituted carbocycle, unsubstituted or substituted heterocycle, —CHR²⁰-(unsubstituted or substituted carbocycle), —CHR²⁰-(unsubstituted or substituted heterocycle), —C(═O)R¹⁹, —CO₂R¹⁹, or —C(═O)NHR¹⁹.

In some embodiments, R¹⁸ is hydrogen, unsubstituted or substituted C₁-C₆ alkyl, unsubstituted or substituted C₁-C₆ fluoroalkyl, unsubstituted or substituted C₁-C₆ heteroalkyl, unsubstituted or substituted C₁-C₆ alkenyl, unsubstituted or substituted C₁-C₆ alkynyl. In some embodiments, R¹⁸ is substituted C₁-C₆ alkyl, unsubstituted or substituted C₁-C₆ fluoroalkyl, or unsubstituted or substituted C₁-C₆ heteroalkyl. In some embodiments, R^{1M} is C₁-C₆ alkyl which is substituted by —CN, —OH, or -Ome. In some embodiments, R¹⁸ is hydrogen.

In some embodiments, R¹⁸ is unsubstituted or substituted carbocycle or unsubstituted or substituted heterocycle. In some embodiments, R¹¹ is unsubstituted or substituted C₃-C₁₀ cycloalkyl, unsubstituted or substituted 3- to 10-membered heterocycloalkyl, unsubstituted or substituted aryl, or unsubstituted or substituted heteroaryl. In some embodiments, R¹⁸ is unsubstituted or substituted C₃-C₆ monocyclic cycloalkyl, unsubstituted or substituted C₅-C₁₀ bicyclic cycloalkyl, or unsubstituted or substituted 3- to 10-membered monocyclic or bicyclic heterocycloalkyl. In some embodiments, R¹⁸ is unsubstituted or substituted 3- to 10-membered monocyclic or bicyclic heterocycloalkyl containing an oxygen atom; or R¹⁸ is unsubstituted or substituted 5- to 10-membered bicyclic heterocycloalkyl which is a bridged, fused, or spirocyclic heterocycloalkyl.

In some embodiments, R¹⁸ is —CHR²⁰-(unsubstituted or substituted carbocycle) or —CHR²⁰-(unsubstituted or substituted heterocycle). In some embodiments, R¹⁸ is —CHR²¹-(unsubstituted or substituted C₃-C₆ cycloalkyl), —CHR²⁰-(unsubstituted or substituted 3- to 6-membered heterocycloalkyl), —CHR²⁰-(unsubstituted or substituted phenyl), or —CHR²⁰-(unsubstituted or substituted 5- or 6-membered heteroaryl); and R²⁰ is hydrogen or —CH₃.

In some embodiments, R¹⁸ is —C(═O)R¹⁹, —CO₂R¹⁹, or —C(═O)NHR¹⁹; and R¹⁹ is unsubstituted or substituted C₁-C₆ alkyl, unsubstituted or substituted C₃-C₁₀ cycloalkyl, unsubstituted or substituted 3- to 10-membered heterocycloalkyl.

In some embodiments, described herein is a compound of Formula (III):

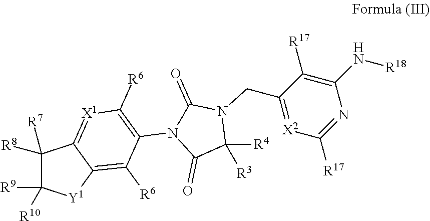

or a pharmaceutically acceptable salt or solvate thereof, wherein:
   X¹ is CR⁶ or N;
      each R⁶ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²¹, —CO₂R²², —N(R²)₂, —C(═O)N(R²²)₂, —NR²²C(═O)R²¹, —S(═O)₂N(R²²)₂, or —NR²²S(═O)₂R²¹;
   Y¹ is —O—, —NR¹¹—, or —SO₂—;
      R¹¹ is hydrogen, C₁-C₆ alkyl, C₁-C₆ fluoroalkyl, —C(═O)NH₂, —C(═O)R¹², or —S(═O)₂R¹²;
      R¹² is unsubstituted or substituted C₁-C₆ alkyl, unsubstituted or substituted C₃-C₆ cycloalkyl, or unsubstituted or substituted 3- to 6-membered heterocycloalkyl;
   R⁷ and R⁸ are each independently hydrogen, —OH, C₁-C₆ alkyl, C₁-C₆ fluoroalkyl, or C₁-C₆ alkoxy;
   or R⁷ and R⁸ are taken together with the carbon atom to which they are attached to form an unsubstituted or substituted C₃-C₆ cycloalkyl or unsubstituted or substituted 3- to 6-membered heterocycloalkyl;
   R⁹ and R¹⁰ are each independently hydrogen, halogen, C₁-C₆ alkyl, C₁-C₆ fluoroalkyl, or C₁-C₆ alkoxy;
   or R⁹ and R¹⁰ are taken together with the carbon atom to which they are attached to form a —C(═O)—;
   or R⁹ and R¹⁰ are taken together with the carbon atom to which they are attached to form an unsubstituted or substituted C₃-C₆ cycloalkyl or unsubstituted or substituted 3- to 6-membered heterocycloalkyl;
R³ is hydrogen, unsubstituted or substituted C₁-C₆ alkyl, unsubstituted or substituted C₁-C₆ fluoroalkyl, unsubstituted or substituted C₁-C₆ alkoxy, unsubstituted or substituted phenyl, unsubstituted or substituted benzyl, unsubstituted or substituted monocyclic heteroaryl, unsubstituted or substituted C₃-C₆ cycloalkyl, or unsubstituted or substituted 3- to 6-membered heterocycloalkyl;
R⁴ is hydrogen or C₁-C₆ alkyl;
or R³ and R⁴ are taken together with the carbon atom to which they are attached to form a C₃-C₆ cycloalkyl or 3- to 6-membered heterocycloalkyl; X² is CR¹⁷ or N;
each R¹⁷ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²¹, —CO₂R²², —N(R²²)₂, —C(═O)N(R²²)₂, —NR²²C(═O)R²¹, —S(═O)₂N(R²²)₂, or —NR²²S(═O)₂R²¹;
R¹⁸ is unsubstituted or substituted C₁-C₆ alkyl, unsubstituted or substituted C₁-C₆ fluoroalkyl, unsubstituted or substituted C₁-C₆ heteroalkyl, unsubstituted or substituted C₁-C₆ alkenyl, unsubstituted or substituted C₁-C₆ alkynyl, unsubstituted or substituted C₃-C₁₀ cycloalkyl, unsubstituted or substituted 3- to 10-membered heterocycloalkyl, —CHR²⁰-(unsubstituted or substituted carbocycle), —CHR²⁰-(unsubstituted or substituted heterocycle), —C(═O)R¹⁹, —CO₂R¹⁹, or —C(═O)NHR¹⁹;
   R¹⁹ is hydrogen, unsubstituted or substituted C₁-C₆ alkyl, unsubstituted or substituted C₃-C₁₀ cycloalkyl, or unsubstituted or substituted 3- to 10-membered heterocycloalkyl;
   R²⁰ is hydrogen or —CH₃;
each R²¹ is independently unsubstituted or substituted C₁-C₆ alkyl, unsubstituted or substituted C₃-C₆ cycloalkyl, unsubstituted or substituted 3- to 6-membered heterocycloalkyl, unsubstituted or substituted aryl, or unsubstituted or substituted heteroaryl;
each R²² is independently hydrogen, unsubstituted or substituted C₁-C₆ alkyl, unsubstituted or substituted C₃-C₆ cycloalkyl, unsubstituted or substituted 3- to 6-membered heterocycloalkyl, unsubstituted or substituted aryl, or unsubstituted or substituted heteroaryl; or two R²² on the same N atom are taken together with the N atom to which they are attached to form an unsubstituted or substituted N-containing heterocycloalkyl;
wherein each substituted alkyl, substituted fluoroalkyl, substituted aryl, substituted heteroaryl, substituted cycloalkyl, and substituted heterocycloalkyl is substituted with one or more R^{s} groups independently selected from the group consisting of halogen, C₁-C₆ alkyl, C₁-C₆ fluoroalkyl, C₃-C₆ cycloalkyl, 3- to 6-membered heterocycloalkyl, phenyl, benzyl, 5-membered heteroaryl, 6-membered heteroaryl, —CN, —OR²³, —CO₂R²³, —C(═O)N(R²³)₂, —N(R²³)₂, —NR²²C(═O)R²⁴, —SR²³, —S(═O)R²⁴, —SO₂R²⁴, or —SO₂N(R²³)₂;
   each R²³ is independently selected from hydrogen, C₁-C₆ alkyl, C₃-C₆ cycloalkyl, 3- to 6-membered heterocycloalkyl, phenyl, benzyl, 5-membered heteroaryl and 6-membered heteroaryl; or two R²³ groups are taken together with the N atom to which they are attached to form a 3- to 6-membered N-containing heterocycloalkyl;
each R²⁴ is independently selected from C₁-C₆ alkyl, C₃-C₆ cycloalkyl, 3- to 6-membered heterocycloalkyl, phenyl, benzyl, 5-membered heteroaryl and 6-membered heteroaryl.

### Additional Embodiments of Formula (III)

In some embodiments, R³ is hydrogen, unsubstituted or substituted C₁-C₆ alkyl, unsubstituted or substituted C₁-C₆ fluoroalkyl, unsubstituted or substituted C₁-C₆ alkoxy, unsubstituted or substituted phenyl, unsubstituted or substituted benzyl, unsubstituted or substituted monocyclic heteroaryl, unsubstituted or substituted C₃-C₆ cycloalkyl, or unsubstituted or substituted 3- to 6-membered heterocycloalkyl. In some embodiments, R³ is hydrogen, unsubstituted or substituted C₁-C₆ alkyl, unsubstituted or substituted C₁-C₆ fluoroalkyl, unsubstituted or substituted phenyl, unsubstituted or substituted benzyl, or unsubstituted or substituted C₃-C₆ cycloalkyl. In some embodiments, R³ is unsubstituted or substituted C₁-C₆ alkyl, phenyl, benzyl, or C₃-C₆ cycloalkyl. In some embodiments, R³ is C₁-C₆ alkyl which is unsubstituted or substituted by —OH, phenyl, benzyl, or C₃-C₆ cycloalkyl. In some embodiments, R³ is methyl, ethyl, propyl, isopropyl, butyl, isobutyl, sec-butyl, phenyl, benzyl, cyclopropyl, cyclobutyl, cyclopentyl, cyclohexyl, or —CH₂OH

In some embodiments, R⁴ is hydrogen or C₁-C₆ alkyl. In some embodiments, R⁴ is hydrogen or C₁-C₄ alkyl. In some embodiments, R⁴ is hydrogen or methyl.

In some embodiments, R³ and R⁴ are taken together with the carbon atom to which they are attached to form a C₃-C₆ cycloalkyl or 3- to 6-membered heterocycloalkyl. In some embodiments, R³ and R⁴ are taken together with the carbon atom to which they are attached to form a C₃-C₆ cycloalkyl. In some embodiments, R³ and R⁴ are taken together with the carbon atom to which they are attached to form a cyclopropyl, cyclobutyl, cyclopentyl, or cyclohexyl.

In some embodiments, R³ is C₁-C₆ alkyl; and R⁴ is C₁-C₆ alkyl; or R¹ and R⁴ are taken together with the carbon atom to which they are attached to form a C₃-C₆ cycloalkyl.

In some embodiments, X¹ is CR⁶ or N. In some embodiments, X¹ is CR⁶. In some embodiments, X¹ is N.

In some embodiments, Y¹ is —O—, —NR¹¹—, or —SO₂—. In some embodiments, Y¹ is —O— or —NR¹¹—. In some embodiments, Y¹ is —O—. In some embodiments, Y¹ is —NR¹¹—. In some embodiments, Y¹ is —SO₂—.

In some embodiments, R¹¹ is hydrogen, C₁-C₆ alkyl, C₁-C₆ fluoroalkyl, —C(═O)NH₂, —C(═O)R¹², or —S(═O)₂R¹². In some embodiments. R¹¹ is hydrogen or C₁-C₆ alkyl. In some embodiments, R¹¹ is hydrogen. In some embodiments, R¹¹ is —C(═O)NH₂, —C(═O)R¹², or —S(═O)₂R¹²,

In some embodiments, R¹² is unsubstituted or substituted C₁-C₆ alkyl, unsubstituted or substituted C₃-C₆ cycloalkyl, or unsubstituted or substituted 3- to 6-membered heterocycloalkyl. In some embodiments, R¹² is unsubstituted or substituted C₁-C₆ alkyl or unsubstituted or substituted C₃-C₆ cycloalkyl. In some embodiments, R¹² is C₁-C₆ alkyl or C₃-C₆ cycloalkyl. In some embodiments, R¹² is C₁-C₆ alkyl. In some embodiments, R¹² is C₃-C₆ cycloalkyl.

In some embodiments, each R⁶ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²², —CO₂R²², —N(R²²)₂, —C(═O)N(R²²)₂, —NR²²C(═O)R²¹. —S(═O)₂N(R²²)₂, or —NR²S(═O)₂R²¹. In some embodiments, each R⁶ is independently hydrogen, halogen, C₁-C₆ alkyl, C₁-C₆ fluoroalkyl, unsubstituted or substituted C₃-C₆ cycloalkyl, unsubstituted or substituted 3- to 6-membered heterocycloalkyl, —CN, —OH, —OR²¹, —CO₂R², —N(R²²)₂, —C(═O)N(R²²)₂, —NR═C(═O)R²², —S(═O)₂N(R²²)₂, or —NR²²S(═O)₂R²¹. In some embodiments, each R⁶ is independently hydrogen, halogen, C₁-C₆ alkyl, C₁-C₆ fluoroalkyl, —CN, —OH, —OR²¹, —N(R²²)₂, or —C(═O)N(R²²)₂. In some embodiments, each R⁶ is independently hydrogen, halogen, or C₁-C₆ alkyl. In some embodiments, each R⁶ is hydrogen.

In some embodiments, R⁷ and R⁸ are each independently hydrogen, —OH, C₁-C₆ alkyl, C₁-C₆ fluoroalkyl, or C₁-C₆ alkoxy. In some embodiments, R⁷ and R⁸ are each independently hydrogen, —OH, or C₁-C₆ alkyl. In some embodiments, R⁷ and R¹ are each independently hydrogen or C₁-C₆ alkyl. In some embodiments, R⁷ and R⁸ are each independently hydrogen or methyl.

In some embodiments, R⁷ and R⁸ are taken together with the carbon atom to which they are attached to form an unsubstituted or substituted C₃-C₆ cycloalkyl or unsubstituted or substituted 3- to 6-membered heterocycloalkyl. In some embodiments, R⁷ and R are taken together with the carbon atom to which they are attached to form an unsubstituted or substituted C₃-C₆ cycloalkyl. In some embodiments, R⁷ and R⁸ are taken together with the carbon atom to which they are attached to form an unsubstituted or substituted C₃-C₄ cycloalkyl. In some embodiments, R⁷ and R⁸ are taken together with the carbon atom to which they are attached to form a 3- to 6-membered heterocycloalkyl. In some embodiments, R⁷ and R⁸ are taken together with the carbon atom to which they are attached to form a 3- or 4-membered heterocycloalkyl.

In some embodiments, R⁹ and R¹⁰ are each independently hydrogen, halogen, C₁-C₆ alkyl, C₁-C₆ fluoroalkyl, or C₁-C₆ alkoxy. In some embodiments, R⁹ and R¹⁰ are each independently hydrogen, halogen, C₁-C₆ alkyl, or C₁-C₆ fluoroalkyl. In some embodiments. R⁹ and R¹⁰ are each independently hydrogen or C₁-C₆ alkyl. In some embodiments, R⁹ and R¹⁰ are each independently hydrogen or methyl.

In some embodiments, R⁹ and R¹⁰ are taken together with the carbon atom to which they are attached to form a —C(═O)—.

In some embodiments, R⁹ and R¹⁰ are taken together with the carbon atom to which they are attached to form an unsubstituted or substituted C₃-C₆ cycloalkyl or unsubstituted or substituted 3- to 6-membered heterocycloalkyl. In some embodiments, R⁹ and R¹⁰ are taken together with the carbon atom to which they are attached to form an unsubstituted or substituted C₃-C₆ cycloalkyl. In some embodiments, R⁹ and R¹⁰ are taken together with the carbon atom to which they are attached to form an unsubstituted or substituted C₃-C₄ cycloalkyl. In some embodiments, R⁹ and R¹⁰ are taken together with the carbon atom to which they are attached to form a 3- to 6-membered heterocycloalkyl. In some embodiments, R⁹ and R¹⁰ are taken together with the carbon atom to which they are attached to form a 3- or 4-membered heterocycloalkyl.

In some embodiments, R¹ is

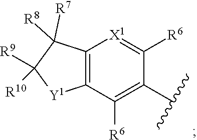

X¹ is CR⁶ or N; and each R⁶ is independently hydrogen, halogen, C₁-C₆ alkyl, C₁-C₆ fluoroalkyl, unsubstituted or substituted C₃-C₆ cycloalkyl, unsubstituted or substituted 3- to 6-membered heterocycloalkyl, —CN, —OH, —OR²¹, —CO₂R²², —N(R²²)₂, —C(═O)N(R²²)₂, —NR²²C(═O)R²¹, —S(═O)₂N(R²²)₂, or —NR²²S(═O)₂R²¹.

In some embodiments, Y¹ is —NR¹¹—; R¹¹ is hydrogen, C₁-C₆ alkyl, C₁-C₆ fluoroalkyl, —C(═O)NH₂, —C(═O)R¹², or —S(═O)₂R¹²; and R¹² is unsubstituted or substituted C₁-C₆ alkyl or unsubstituted or substituted C₃-C₆ cycloalkyl.

In some embodiments, R⁷ and R⁸ are each independently hydrogen, —OH, C₁-C₆ alkyl, C₁-C₆ fluoroalkyl, or C₁-C₆ alkoxy; or R⁷ and R⁸ are taken together with the carbon atom to which they are attached to form an unsubstituted or substituted C₃-C₄ cycloalkyl; R⁹ and R¹⁰ are each independently hydrogen, halogen, C₁-C₆ alkyl, C₁-C₆ fluoroalkyl, or C₁-C₆ alkoxy; or R⁹ and R¹⁰ are taken together with the carbon atom to which they are attached to form a —C(═O)—; or R⁹ and R¹⁰ are taken together with the carbon atom to which they are attached to form an unsubstituted or substituted C₃-C₄ cycloalkyl.

In some embodiments, X² is CR¹⁷ or N. In some embodiments, X² is CR¹⁷. In some embodiments, X² is N.

In some embodiments, each R⁷ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²¹, —CO₂R²², —N(R²²)₂, —C(═O)N(R²²)₂, —OC(═O)R²¹, —NR²²C(═O)R²¹; —S(═O)₂N(R²²)₂, or —NR²²S(═O)₂R²¹. In some embodiments, each R¹⁷ is independently hydrogen, halogen, unsubstituted or substituted C₁-C₆ alkyl, unsubstituted or substituted C₁-C₆ fluoroalkyl, —CN, —OH, —OR²¹, —CO₂R²², —N(R²²)₂, —C(═O)N(R²²)₂, —OC(═O)R²¹, —NR²C(═O)R²¹, —S(═O)₂N(R²²)₂, or —NR²²S(═O)₂R²¹. In some embodiments, each R¹⁷ is independently hydrogen, halogen, C₁-C₆ alkyl, C₁-C₆ fluoroalkyl, —CN, —OH, —OR², —N(R²²)₂, or —C(═O)N(R²²)₂. In some embodiments, each R¹⁷ is independently hydrogen, halogen, C₁-C₆ alkyl, or C₁-C₆ fluoroalkyl. In some embodiments, each R¹⁷ is hydrogen.

In some embodiments, X² is CR¹⁷; and each R¹⁷ is independently hydrogen, halogen. C₁-C₆ alkyl, or C₁-C₆ fluoroalkyl.

In some embodiments, X² is CR¹⁷; and each R¹⁷ is hydrogen.

In some embodiments, R¹⁸ is unsubstituted or substituted C₁-C₆ alkyl, unsubstituted or substituted C₁-C₆ fluoroalkyl, unsubstituted or substituted C₁-C₆ heteroalkyl, unsubstituted or substituted C₁-C₆ alkenyl, unsubstituted or substituted C₁-C₆ alkynyl, unsubstituted or substituted C₃-C₁₀ cycloalkyl, unsubstituted or substituted 3- to 10-membered heterocycloalkyl, —CHR²⁰-(unsubstituted or substituted carbocycle), —CHR²⁰-(unsubstituted or substituted heterocycle), —C(═O)R¹⁹, —CO₂R¹⁹, or —C(═O)NHR¹⁹.

In some embodiments, R¹⁸ is unsubstituted or substituted C₁-C₆ alkyl, unsubstituted or substituted C₁-C₆ fluoroalkyl, unsubstituted or substituted C₁-C₆ heteroalkyl, unsubstituted or substituted C₁-C₆ alkenyl, unsubstituted or substituted C₁-C₆ alkynyl. In some embodiments, R^{1′} is substituted C₁-C₆ alkyl, unsubstituted or substituted C₁-C₆ fluoroalkyl, or unsubstituted or substituted C₁-C₆ heteroalkyl. In some embodiments, R¹⁸ is C₁-C₆ alkyl which is substituted by —CN, —OH, or -Ome.

In some embodiments, R¹⁸ is unsubstituted or substituted C₃-C₁₀ cycloalkyl or unsubstituted or substituted 3- to 10-membered heterocycloalkyl. In some embodiments, R¹⁸ is unsubstituted or substituted C₃-C₆ monocyclic cycloalkyl, unsubstituted or substituted C₅-C₁₀ bicyclic cycloalkyl, or unsubstituted or substituted 3- to 10-membered monocyclic or bicyclic heterocycloalkyl. In some embodiments, R¹⁸ is unsubstituted or substituted 3- to 10-membered monocyclic or bicyclic heterocycloalkyl containing an oxygen atom; or R¹⁸ is unsubstituted or substituted 5- to 10-membered bicyclic heterocycloalkyl which is a bridged, fused, or spirocyclic heterocycloalkyl.

In some embodiments, R¹⁸ is —CHR²⁰-(unsubstituted or substituted carbocycle) or —CHR²⁰-(unsubstituted or substituted heterocycle). In some embodiments, R¹⁸ is —CHR²⁰-(unsubstituted or substituted C₃-C₆ cycloalkyl), —CHR²⁰-(unsubstituted or substituted 3- to 6-membered heterocycloalkyl), —CHR²⁰-(unsubstituted or substituted phenyl), or —CHR²⁰-(unsubstituted or substituted 5- or 6-membered heteroaryl); and R²⁰ is hydrogen or —CH₃.

In some embodiments, R¹⁸ is —C(═O)R¹⁹, —CO₂R¹⁹, or —C(═O)NHR¹⁹; and R¹⁹ is unsubstituted or substituted C₁-C₆ alkyl, unsubstituted or substituted C₃-C₁₀ cycloalkyl, unsubstituted or substituted 3- to 10-membered heterocycloalkyl.

In some embodiments, described herein is a compound of Formula (IV):

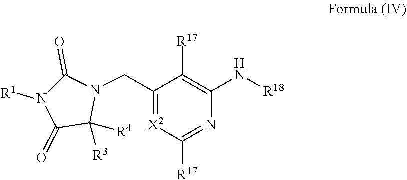

or a pharmaceutically acceptable salt or solvate thereof, wherein:
R¹ is unsubstituted or substituted bicyclic heteroaryl, wherein if R¹ is substituted, then it is substituted with R^{A} and 1-3 R⁵;
   R^{A} is unsubstituted or substituted C₁-C₆ alkyl, unsubstituted or substituted C₁-C₆ fluoroalkyl, unsubstituted or substituted C₃-C₆ cycloalkyl, unsubstituted or substituted 3- to 6-membered heterocycloalkyl, halogen, —CN, —SR^{A1}, —S(═O)R^{A1}, —S(═O)₂R^{A1}, —OR^{A1}, —SF₅, or —SiMe₃;
      R^{A1} is C₁-C₆ alkyl, C₁-C₆ fluoroalkyl, C₃-C₆ cycloalkyl, or 3- to 6-membered heterocycloalkyl;
   each R⁵ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²¹, —CO₂R²², —N(R²²)₂, —C(═O)N(R²²)₂, —NR²²C(═O)R²¹, —S(═O)₂N(R²²)₂, or —NR²²S(═O)₂R²¹
R³ is hydrogen, unsubstituted or substituted C₁-C₆ alkyl, unsubstituted or substituted C₁-C₆ fluoroalkyl, unsubstituted or substituted C₁-C₆ alkoxy, unsubstituted or substituted phenyl, unsubstituted or substituted benzyl, unsubstituted or substituted monocyclic heteroaryl, unsubstituted or substituted C₃-C₆ cycloalkyl, or unsubstituted or substituted 3- to 6-membered heterocycloalkyl;
R⁴ is hydrogen or C₁-C₆ alkyl;
or R³ and R⁴ are taken together with the carbon atom to which they are attached to form a C₃-C₆ cycloalkyl or 3- to 6-membered heterocycloalkyl;
X² is CR¹⁷ or N;
each R¹⁷ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²¹, —CO₂R²², —N(R²²)₂, —C(═O)N(R²²)₂, —NR²²C(═O)R²¹, —S(═O)₂N(R²²)₂, or —NR²²S(═O)₂R²¹;
R¹⁸ is hydrogen, unsubstituted or substituted C₁-C₆ alkyl, unsubstituted or substituted C₁-C₆ fluoroalkyl, unsubstituted or substituted C₁-C₆ heteroalkyl, unsubstituted or substituted C₁-C₆ alkenyl, unsubstituted or substituted C₁-C₆ alkynyl, unsubstituted or substituted carbocycle, unsubstituted or substituted heterocycle, —CHR²⁰-(unsubstituted or substituted carbocycle), —CHR²⁰-(unsubstituted or substituted heterocycle), —C(═O)R¹⁹, —CO₂R¹⁹, or —C(═O)NHR¹⁹;
   R¹⁹ is hydrogen, unsubstituted or substituted C₁-C₆ alkyl, unsubstituted or substituted C₃-C₁₀ cycloalkyl, or unsubstituted or substituted 3- to 10-membered heterocycloalkyl;
   R²⁰ is hydrogen or —CH₃:
each R²¹ is independently unsubstituted or substituted C₁-C₆ alkyl, unsubstituted or substituted C₃-C₆ cycloalkyl, unsubstituted or substituted 3- to 6-membered heterocycloalkyl, unsubstituted or substituted aryl, or unsubstituted or substituted heteroaryl;
each R²² is independently hydrogen, unsubstituted or substituted C₁-C₆ alkyl, unsubstituted or substituted C₃-C₆ cycloalkyl, unsubstituted or substituted 3- to 6-membered heterocycloalkyl, unsubstituted or substituted aryl, or unsubstituted or substituted heteroaryl; or two R² on the same N atom are taken together with the N atom to which they are attached to form an unsubstituted or substituted N-containing heterocycloalkyl;
wherein each substituted alkyl, substituted fluoroalkyl, substituted aryl, substituted heteroaryl, substituted cycloalkyl, and substituted heterocycloalkyl is substituted with one or more R^{s} groups independently selected from the group consisting of halogen, C₁-C₆ alkyl, C₁-C₆ fluoroalkyl, C₃-C₆ cycloalkyl, 3- to 6-membered heterocycloalkyl, phenyl, benzyl, 5-membered heteroaryl, 6-membered heteroaryl, —CN, —OR²³, —CO₂R²³—, —C(═O)N(R²³)₂, —N(R²³)₂, —NR²³C(═O)R²⁴, —SR²³, —S(═O)R²⁴, —SO₂R²⁴, or —SO₂N(R²³)₂;
   each R²³ is independently selected from hydrogen, C₁-C₆ alkyl, C₃-C₆ cycloalkyl, 3- to 6-membered heterocycloalkyl, phenyl, benzyl, 5-membered heteroaryl and 6-membered heteroaryl; or two R²³ groups are taken together with the N atom to which they are attached to form a 3- to 6-membered N-containing heterocycloalkyl;
   each R²⁴ is independently selected from C₁-C₆ alkyl, C₃-C₆ cycloalkyl, 3- to 6-membered heterocycloalkyl, phenyl, benzyl, 5-membered heteroaryl and 6-membered heteroaryl.

### Additional Embodiments of Formula (IV)

In some embodiments, R³ is hydrogen, unsubstituted or substituted C₁-C₆ alkyl, unsubstituted or substituted C₁-C₆ fluoroalkyl, unsubstituted or substituted C₁-C₆ alkoxy, unsubstituted or substituted phenyl, unsubstituted or substituted benzyl, unsubstituted or substituted monocyclic heteroaryl, unsubstituted or substituted C₃-C₆ cycloalkyl, or unsubstituted or substituted 3- to 6-membered heterocycloalkyl. In some embodiments, R³ is hydrogen, unsubstituted or substituted C₁-C₆ alkyl, unsubstituted or substituted C₁-C₆ fluoroalkyl, unsubstituted or substituted phenyl, unsubstituted or substituted benzyl, or unsubstituted or substituted C₃-C₆ cycloalkyl. In some embodiments, R³ is unsubstituted or substituted C₁-C₆ alkyl, phenyl, benzyl, or C₃-C₆ cycloalkyl. In some embodiments, R³ is C₁-C₆ alkyl which is unsubstituted or substituted by —OH, phenyl, benzyl, or C₃-C₆ cycloalkyl. In some embodiments, R³ is methyl, ethyl, propyl, isopropyl, butyl, isobutyl, sec-butyl, phenyl, benzyl, cyclopropyl, cyclobutyl, cyclopentyl, cyclohexyl, or —CH₂OH

In some embodiments, R⁴ is hydrogen or C₁-C₆ alkyl. In some embodiments, R⁴ is hydrogen or C₁-C₄ alkyl. In some embodiments, R⁴ is hydrogen or methyl.

In some embodiments, R³ and R⁴ are taken together with the carbon atom to which they are attached to form a C₃-C₆ cycloalkyl or 3- to 6-membered heterocycloalkyl. In some embodiments, R³ and R⁴ are taken together with the carbon atom to which they are attached to form a C₃-C₆ cycloalkyl. In some embodiments, R³ and R⁴ are taken together with the carbon atom to which they are attached to form a cyclopropyl, cyclobutyl, cyclopentyl, or cyclohexyl.

In some embodiments, R³ is C₁-C₆ alkyl; and R⁴ is C₁-C₆ alkyl; or R³ and R⁴ are taken together with the carbon atom to which they are attached to form a C₃-C₆ cycloalkyl.

In some embodiments, each R⁵ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²², —CO₂R²², —N(R²²)₂, —C(═O)N(R²²)₂—NR²²C(═O)R²¹, —S(═O)₂N(R²²)₂, or —NR²²S(═O)₂R²¹. In some embodiments, each R⁵ is independently hydrogen, halogen, unsubstituted or substituted C₁-C₆ alkyl, unsubstituted or substituted C₁-C₆ fluoroalkyl, unsubstituted or substituted C₃-C₆ cycloalkyl, unsubstituted or substituted 3- to 6-membered heterocycloalkyl. —CN. —OH, —OR²¹, —N(R²²)₂, or —C(═O)N(R²²)₂. In some embodiments, each R⁵ is independently hydrogen, halogen, C₁-C₆ alkyl, C₁-C₆ fluoroalkyl, C₃-C₆ cycloalkyl, 3- to 6-membered heterocycloalkyl, —CN, or —OR²¹. In some embodiments, each R⁵ is independently hydrogen or C₁-C₆ alkyl. In some embodiments, each R⁵ is hydrogen.

In some embodiments, R^{A} is unsubstituted or substituted C₁-C₆ alkyl, unsubstituted or substituted C₁-C₆ fluoroalkyl, unsubstituted or substituted C₃-C₆ cycloalkyl, unsubstituted or substituted 3- to 6-membered heterocycloalkyl, halogen, —CN, —SR^{A1}, —S(═O)R^{A1}, —S(═O)₂R^{A1}, —OR^{A1}, —SF₅, or —SiMe₃; and R^{A1} is C₁-C₆ alkyl, C₁-C₆ fluoroalkyl, C₃-C₆ cycloalkyl, or 3- to 6-membered heterocycloalkyl. In some embodiments, R^{A} is C₁-C₄ alkyl, C₁-C₄ fluoroalkyl, C₃-C₆ cycloalkyl, 3- to 6-membered heterocycloalkyl, halogen, —CN, —SR^{A1}, —S(═O)R^{A1}, —S(═O)₂R^{A1}, —OR^{A1}, —SF₅, or —SiMe₃; R^{A1} is C₁-C₆ alkyl, C₁-C₆ fluoroalkyl. C₃-C₆ cycloalkyl, or 3- to 6-membered heterocycloalkyl. In some embodiments, R^{A} is C₁-C₄ alkyl, C₁-C₄ fluoroalkyl, or C₃-C₆ cycloalkyl.

In some embodiments, R^{A} is C₁-C₄ alkyl, C₁-C₄ fluoroalkyl, C₃-C₆ cycloalkyl, 3- to 6-membered heterocycloalkyl, halogen, —CN, —SR^{A1}, —S(═O)R^{A1}, —S(═O)₂R^{A1}, —OR^{A1}, —SF₅, or —SiMe₃; R^{A1} is C₁-C₆ alkyl, C₁-C₆ fluoroalkyl, C₃-C₆ cycloalkyl, or 3- to 6-membered heterocycloalkyl; and each R⁵ is independently hydrogen, halogen, C₁-C₆ alkyl, C₁-C₆ fluoroalkyl, C₃-C₆ cycloalkyl, 3- to 6-membered heterocycloalkyl, —CN, or —OR²¹.

In some embodiments, R^{A} is C₁-C₄ alkyl, C₁-C₄ fluoroalkyl, or C₃-C₆ cycloalkyl; and each R⁵ is independently hydrogen or C₁-C₆ alkyl.

In some embodiments, X² is CR¹⁷ or N. In some embodiments, X² is CR¹⁷. In some embodiments, X² is N.

In some embodiments, each R⁷ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²¹, —CO₂R²², —N(R²²)₂, —C(═O)N(R²²)₂, —OC(═O)R²¹, —NR²²C(═O)R²¹, —S(═O)₂N(R²²)₂, or —NR²²S(═O)₂R²¹. In some embodiments, each R¹⁷ is independently hydrogen, halogen, unsubstituted or substituted C₁-C₆ alkyl, unsubstituted or substituted C₁-C₆ fluoroalkyl, —CN, —OH, —OR²¹, —CO₂R²², —N(R²²)₂, —C(═O)N(R²²)₂, —OC(═O)R²¹, —NR²²C(═O)R²¹, —S(═O)₂N(R²²)₂, or —NR²²S(═O)₂R²¹. In some embodiments, each R¹⁷ is independently hydrogen, halogen, C₁-C₆ alkyl, C₁-C₆ fluoroalkyl, —CN, —OH, —OR²¹, —N(R²²)₂, or —C(═O)N(R²²)₂. In some embodiments, each R¹⁷ is independently hydrogen, halogen, C₁—C alkyl, or C₁-C₆ fluoroalkyl. In some embodiments, each R¹⁷ is hydrogen.

In some embodiments, X² is CR¹⁷; and each R¹⁷ is independently hydrogen, halogen, C₁-C₆ alkyl, or C₁-C₆ fluoroalkyl.

In some embodiments, X² is CR¹⁷; and each R¹⁷ is hydrogen.

In some embodiments, R¹⁸ is hydrogen, unsubstituted or substituted C₁-C₆ alkyl, unsubstituted or substituted C₁-C₆ fluoroalkyl, unsubstituted or substituted C₁-C₆ heteroalkyl, unsubstituted or substituted C₁-C₆ alkenyl, unsubstituted or substituted C₁-C₆ alkynyl, unsubstituted or substituted carbocycle, unsubstituted or substituted heterocycle, —CHR²⁰-(unsubstituted or substituted carbocycle), —CHR²⁰-(unsubstituted or substituted heterocycle), —C(═O)R¹⁹, —CO₂R¹⁹, or —C(═O)NHR¹⁹.

In some embodiments, R¹⁸ is hydrogen, unsubstituted or substituted C₁-C₆ alkyl, unsubstituted or substituted C₁-C₆ fluoroalkyl, unsubstituted or substituted C₁-C₆ heteroalkyl, unsubstituted or substituted C₁-C₆ alkenyl, unsubstituted or substituted C₁-C₆ alkynyl. In some embodiments, R¹⁸ is substituted C₁-C₆ alkyl, unsubstituted or substituted C₁-C₆ fluoroalkyl, or unsubstituted or substituted C₁-C₆ heteroalkyl. In some embodiments, R¹⁸ is C₁-C₆ alkyl which is substituted by —CN, —OH, or -Ome. In some embodiments, R¹⁸ is hydrogen.

In some embodiments, R¹⁸ is unsubstituted or substituted carbocycle or unsubstituted or substituted heterocycle. In some embodiments, R¹⁸ is unsubstituted or substituted C₃-C₁₀ cycloalkyl, unsubstituted or substituted 3- to 10-membered heterocycloalkyl, unsubstituted or substituted aryl, or unsubstituted or substituted heteroaryl. In some embodiments, R¹⁸ is unsubstituted or substituted C₃-C₆ monocyclic cycloalkyl, unsubstituted or substituted C₅-C₁₀ bicyclic cycloalkyl, or unsubstituted or substituted 3- to 10-membered monocyclic or bicyclic heterocycloalkyl. In some embodiments, R¹⁸ is unsubstituted or substituted 3- to 10-membered monocyclic or bicyclic heterocycloalkyl containing an oxygen atom; or R¹⁸ is unsubstituted or substituted 5- to 10-membered bicyclic heterocycloalkyl which is a bridged, fused, or spirocyclic heterocycloalkyl.

In some embodiments, R¹⁸ is —CHR²⁰-(unsubstituted or substituted carbocycle) or —CHR²⁰-(unsubstituted or substituted heterocycle). In some embodiments, R⁵ is —CHR²⁰-(unsubstituted or substituted C₃-C₆ cycloalkyl), —CHR²⁰-(unsubstituted or substituted 3- to 6-membered heterocycloalkyl), —CHR²⁰-(unsubstituted or substituted phenyl), or —CHR²⁰-(unsubstituted or substituted 5- or 6-membered heteroaryl); and R²⁰ is hydrogen or —CH₃.

In some embodiments, R¹⁸ is —C(═O)R¹⁹, —CO₂R¹⁹, or —C(═O)NHR¹⁹; and R¹⁹ is unsubstituted or substituted C₁-C₆ alkyl, unsubstituted or substituted C₃-C₁₀ cycloalkyl, unsubstituted or substituted 3- to 10-membered heterocycloalkyl.

In some embodiments, X² is CR¹⁷; and each R¹⁷ is hydrogen.

In some embodiments, R¹⁸ is hydrogen, unsubstituted or substituted C₁-C₆ alkyl, unsubstituted or substituted C₁-C₆ fluoroalkyl, unsubstituted or substituted C₁-C₆ heteroalkyl, unsubstituted or substituted C₁-C₆ alkenyl, unsubstituted or substituted C₁-C₆ alkynyl; or R¹⁸ is substituted C₁-C₆ alkyl, unsubstituted or substituted C₁-C₆ fluoroalkyl, unsubstituted or substituted C₁-C₆ heteroalkyl; or R¹⁸ is C₁-C₆ alkyl which is substituted by —CN, —OH, or -Ome; or R¹⁸ is unsubstituted or substituted C₅-C₁₀ cycloalkyl, unsubstituted or substituted 3- to 10-membered heterocycloalkyl, unsubstituted or substituted aryl, or unsubstituted or substituted heteroaryl; or R¹⁸ is unsubstituted or substituted C₃-C₆ monocyclic cycloalkyl, unsubstituted or substituted C₅-C₁₀ bicyclic cycloalkyl, or unsubstituted or substituted 3- to 10-membered monocyclic or bicyclic heterocycloalkyl; or R¹¹ is unsubstituted or substituted 3- to 10-membered monocyclic or bicyclic heterocycloalkyl containing an oxygen atom; or R¹⁸ is unsubstituted or substituted 5- to 10-membered bicyclic heterocycloalkyl which is a bridged, fused, or spirocyclic heterocycloalkyl; or R¹⁸ is —CHR²⁰-(unsubstituted or substituted C₃-C₆ cycloalkyl), —CHR²⁰-(unsubstituted or substituted 3- to 6-membered heterocycloalkyl), —CHR²⁰-(unsubstituted or substituted phenyl), or —CHR²¹-(unsubstituted or substituted 5- or 6-membered heteroaryl); and R²⁰ is hydrogen or —CH₃; or R¹⁸ is —C(═O)R¹⁹, —CO₂R¹⁹, or —C(═O)NHR¹⁹; and R¹⁹ is unsubstituted or substituted C₁-C₆ alkyl, unsubstituted or substituted C₃-C₁₀ cycloalkyl, unsubstituted or substituted 3- to 10-membered heterocycloalkyl.

In some embodiments, described herein is a compound of Formula (V):

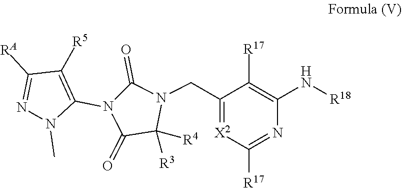

or a pharmaceutically acceptable salt or solvate thereof, wherein:
R^{A} is unsubstituted or substituted C₁-C₆ alkyl, unsubstituted or substituted C₁-C₆ fluoroalkyl, unsubstituted or substituted C₃-C₆ cycloalkyl, unsubstituted or substituted 3- to 6-membered heterocycloalkyl, halogen, —CN, —SR^{A1}, —S(═O)R^{A1}, —S(═O)₂R^{A1}, —OR^{A1}, —SF₅, or —SiMe₃;
   R^{A1} is C₁-C₆ alkyl, C₁-C₆ fluoroalkyl, C₃-C₆ cycloalkyl, or 3- to 6-membered heterocycloalkyl;
R³ is hydrogen, unsubstituted or substituted C₁-C₆ alkyl, unsubstituted or substituted C₁-C₆ fluoroalkyl, unsubstituted or substituted C₁-C₆ alkoxy, unsubstituted or substituted phenyl, unsubstituted or substituted benzyl, unsubstituted or substituted monocyclic heteroaryl, unsubstituted or substituted C₃-C₆ cycloalkyl, or unsubstituted or substituted 3- to 6-membered heterocycloalkyl;
R⁴ is hydrogen or C₁-C₆ alkyl;
or R³ and R⁴ are taken together with the carbon atom to which they are attached to form a C₃-C₆ cycloalkyl or 3- to 6-membered heterocycloalkyl;
R⁵ is hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²¹, —CO₂R²²—N(R²²)₂, —C(═O)N(R²²)₂, —NR²²C(═O)R²¹. —S(═O)₂N(R²²)₂, or —NR²²S(═O)₂R²¹;
X² is CR¹⁷ or N;
each R¹⁷ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²¹, —CO₂R²², —N(R²²)₂, —C(═O)N(R²²)₂, —NR²²C(═O)R²¹, —S(═O)₂N(R²²)₂, or —NR²²S(═O)₂R²¹;
R¹⁸ is hydrogen, unsubstituted or substituted C₁-C₆ alkyl, unsubstituted or substituted C₁-C₆ fluoroalkyl, unsubstituted or substituted C₁-C₆ heteroalkyl, unsubstituted or substituted C₁-C₆ alkenyl, unsubstituted or substituted C₁-C₆ alkynyl, unsubstituted or substituted carbocycle, unsubstituted or substituted heterocycle, —CHR²⁰-(unsubstituted or substituted carbocycle), —CHR²-(unsubstituted or substituted heterocycle), —C(═O)R¹⁹, —CO₂R¹⁹, or —C(═O)NHR¹⁹.
   R¹⁹ is hydrogen, unsubstituted or substituted C₁-C₆ alkyl, unsubstituted or substituted C₃-C₁₀ cycloalkyl, or unsubstituted or substituted 3- to 10-membered heterocycloalkyl;
   R²⁰ is hydrogen or —CH₃;
each R²¹ is independently unsubstituted or substituted C₁-C₆ alkyl, unsubstituted or substituted C₃-C₆ cycloalkyl, unsubstituted or substituted 3- to 6-membered heterocycloalkyl, unsubstituted or substituted aryl, or unsubstituted or substituted heteroaryl;
each R²² is independently hydrogen, unsubstituted or substituted C₁-C₆ alkyl, unsubstituted or substituted C₃-C₆ cycloalkyl, unsubstituted or substituted 3- to 6-membered heterocycloalkyl, unsubstituted or substituted aryl, or unsubstituted or substituted heteroaryl; or two R²² on the same N atom are taken together with the N atom to which they are attached to form an unsubstituted or substituted N-containing heterocycloalkyl;
wherein each substituted alkyl, substituted fluoroalkyl, substituted aryl, substituted heteroaryl, substituted cycloalkyl, and substituted heterocycloalkyl is substituted with one or more R^{s} groups independently selected from the group consisting of halogen, C₁-C₆ alkyl, C₁-C₆ fluoroalkyl, C₃-C₆ cycloalkyl, 3- to 6-membered heterocycloalkyl, phenyl, benzyl, 5-membered heteroaryl, 6-membered heteroaryl, —CN, —OR²³, —CO₂R²³, —C(═O)N(R²³)₂, —N(R²³)₂, —NR²³C(═O)R²⁴, —SR²³, —S(═O)R²⁴, —SO₂R²⁴, or —SO₂N(R²³)₂;
   each R²³ is independently selected from hydrogen, C₁-C₆ alkyl, C₃-C₆ cycloalkyl, 3- to 6-membered heterocycloalkyl, phenyl, benzyl, 5-membered heteroaryl and 6-membered heteroaryl; or two R² groups are taken together with the N atom to which they are attached to form a 3- to 6-membered N-containing heterocycloalkyl;
   each R²⁴ is independently selected from C₁-C₆ alkyl, C₃-C₆ cycloalkyl, 3- to 6-membered heterocycloalkyl, phenyl, benzyl, 5-membered heteroaryl and 6-membered heteroaryl.

### Additional Embodiments of Formula (V)

In some embodiments, R³ is hydrogen, unsubstituted or substituted C₁-C₆ alkyl, unsubstituted or substituted C₁-C₆ fluoroalkyl, unsubstituted or substituted C₁-C₆ alkoxy, unsubstituted or substituted phenyl, unsubstituted or substituted benzyl, unsubstituted or substituted monocyclic heteroaryl, unsubstituted or substituted C₃-C₆ cycloalkyl, or unsubstituted or substituted 3- to 6-membered heterocycloalkyl. In some embodiments, R³ is hydrogen, unsubstituted or substituted C₁-C₆ alkyl, unsubstituted or substituted C₁-C₆ fluoroalkyl, unsubstituted or substituted phenyl, unsubstituted or substituted benzyl, or unsubstituted or substituted C₃-C₆ cycloalkyl. In some embodiments, R³ is unsubstituted or substituted C₁-C₆ alkyl, phenyl, benzyl, or C₃-C₆ cycloalkyl. In some embodiments, R³ is C₁-C₆ alkyl which is unsubstituted or substituted by —OH, phenyl, benzyl, or C₃-C₆ cycloalkyl. In some embodiments, R³ is methyl, ethyl, propyl, isopropyl, butyl, isobutyl, sec-butyl, phenyl, benzyl, cyclopropyl, cyclobutyl, cyclopentyl, cyclohexyl, or —CH₂OH

In some embodiments, R⁴ is hydrogen or C₁-C₆ alkyl. In some embodiments, R⁴ is hydrogen or C₁-C₄ alkyl. In some embodiments, R⁴ is hydrogen or methyl.

In some embodiments, R³ and R⁴ are taken together with the carbon atom to which they are attached to form a C₃-C₆ cycloalkyl or 3- to 6-membered heterocycloalkyl. In some embodiments, R³ and R⁴ are taken together with the carbon atom to which they are attached to form a C₃-C₆ cycloalkyl. In some embodiments, R³ and R⁴ are taken together with the carbon atom to which they are attached to form a cyclopropyl, cyclobutyl, cyclopentyl, or cyclohexyl.

In some embodiments, R³ is C₁-C₆ alkyl; and R⁴ is C₁-C₆ alkyl; or R³ and R⁴ are taken together with the carbon atom to which they are attached to form a C₃-C₆ cycloalkyl.

In some embodiments, each R⁵ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²², —CO₂R²², —N(R²²)₂, —C(═O)N(R²²)₂, —NR²²C(═O)R²¹, —S(═O)₂N(R²²)₂, or —NR²²S(═O)₂R²¹. In some embodiments, each R⁵ is independently hydrogen, halogen, unsubstituted or substituted C₁-C₆ alkyl, unsubstituted or substituted C₁-C₆ fluoroalkyl, unsubstituted or substituted C₃-C₆ cycloalkyl, unsubstituted or substituted 3- to 6-membered heterocycloalkyl, —CN, —OH, —OR²¹, —N(R²²)₂, or —C(═O)N(R²²)₂. In some embodiments, each R⁵ is independently hydrogen, halogen, C₁-C₆ alkyl, C₁-C₆ fluoroalkyl, C₃-C₆ cycloalkyl, 3- to 6-membered heterocycloalkyl, —CN, or —OR²¹. In some embodiments, each R⁵ is independently hydrogen or C₁-C₆ alkyl. In some embodiments, each R⁵ is hydrogen.

In some embodiments, R^{A} is unsubstituted or substituted C₁-C₆ alkyl, unsubstituted or substituted C₁-C₆ fluoroalkyl, unsubstituted or substituted C₃-C₆ cycloalkyl, unsubstituted or substituted 3- to 6-membered heterocycloalkyl, halogen, —CN, —SR^{A1}, —S(═O)R^{A1}, —S(═O)₂R^{A1}, —OR^{A1}, —SF₅, or —SiMe₃; and R^{A1} is C₁-C₆ alkyl, C₁-C₆ fluoroalkyl, C₃-C₆ cycloalkyl, or 3- to 6-membered heterocycloalkyl. In some embodiments, R^{A} is C₁-C₄ alkyl, C₁-C₄ fluoroalkyl, C₃-C₆ cycloalkyl, 3- to 6-membered heterocycloalkyl, halogen, —CN, —SR^{A1}, —S(═O)R^{A1}, —S(═O)₂R^{A1}, —OR^{A1}, —SF₅, or —SiMe₃; and R^{A1} is C₁-C₆ alkyl, C₁-C₆ fluoroalkyl, C₃-C₆ cycloalkyl, or 3- to 6-membered heterocycloalkyl. In some embodiments, R^{A} is C₁-C₄ alkyl, C₁-C₄ fluoroalkyl, or C₃-C₆ cycloalkyl. In some embodiments, R^{A} is C₁-C₄ alkyl. In some embodiments, R^{A} is iso-propyl or t-butyl. In some embodiments, R^{A} is iso-propyl. In some embodiments, R^{A} is t-butyl.

In some embodiments, R^{A} is C₁-C₄ alkyl, C₁-C₄ fluoroalkyl, C₃-C₆ cycloalkyl, 3- to 6-membered heterocycloalkyl, halogen, —CN, —SR^{A1}, —S(═O)R^{A1}, —S(═O)₂R^{A1}, —OR^{A1}, —SF₅, or —SiMe₃; R^{A1} is C₁-C₆ alkyl, C₁-C₆ fluoroalkyl, C₃-C₆ cycloalkyl, or 3- to 6-membered heterocycloalkyl; and each R⁵ is independently hydrogen, halogen, C₁-C₆ alkyl, C₁-C₆ fluoroalkyl, C₃-C₆ cycloalkyl, 3- to 6-membered heterocycloalkyl, —CN, or —OR²¹. In some embodiments, R^{A} is C₁-C₄ alkyl, C₁-C₄ fluoroalkyl, or C₃-C₆ cycloalkyl; and each R⁵ is independently hydrogen or C₁-C₆ alkyl.

In some embodiments, X² is CR¹⁷ or N. In some embodiments, X² is CR¹⁷. In some embodiments, X² is N.

In some embodiments, each R⁷ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²¹, —CO₂R²², —N(R²²)₂, —C(═O)N(Rn)², —OC(═O)R²¹, —NR²²C(═O)R²¹, —S(═O)₂N(R²²)₂, or —NR²²S(═O)₂R²¹. In some embodiments, each R¹⁷ is independently hydrogen, halogen, unsubstituted or substituted C₁-C₆ alkyl, unsubstituted or substituted C₁-C₆ fluoroalkyl, —CN, —OH, —OR²¹, —CO₂R²², —N(R²²)₂, —C(═O)N(R²²)₂, —OC(═O)R²¹, —NR²²C(═O)R²¹, —S(═O)₂N(R²²)₂, or —NR²²S(═O)R²¹. In some embodiments, each R¹⁷ is independently hydrogen, halogen, C₁-C₆ alkyl, C₁-C₆ fluoroalkyl, —CN, —OH, —OR²¹, —N(R²)₂, or —C(═O)N(R²²)₂. In some embodiments, each R¹⁷ is independently hydrogen, halogen, C₁-C₆ alkyl, or C₁-C₆ fluoroalkyl. In some embodiments, each R¹⁷ is hydrogen.

In some embodiments, X² is CR¹⁷; and each R¹⁷ is independently hydrogen, halogen, C₁-C₆ alkyl, or C₁-C₆ fluoroalkyl.

In some embodiments, X² is CR¹⁷; and each R¹⁷ is hydrogen.

In some embodiments, R¹⁸ is hydrogen, unsubstituted or substituted C₁-C₆ alkyl, unsubstituted or substituted C₁-C₆ fluoroalkyl, unsubstituted or substituted C₁-C₆ heteroalkyl, unsubstituted or substituted C₁-C₆ alkenyl, unsubstituted or substituted C₁-C₆ alkynl, unsubstituted or substituted carbocycle, unsubstituted or substituted heterocycle, —CHR²⁰-(unsubstituted or substituted carbocycle), —CHR²⁰-(unsubstituted or substituted heterocycle), —C(═O)R¹⁹, —CO₂R¹⁹, or —C(═O)NHR¹⁹.

In some embodiments, R¹⁸ is hydrogen, unsubstituted or substituted C₁-C₆ alkyl, unsubstituted or substituted C₁-C₆ fluoroalkyl, unsubstituted or substituted C₁-C₆ heteroalkyl, unsubstituted or substituted C₁-C₆ alkenyl, unsubstituted or substituted C₁-C₆ alkynyl. In some embodiments, R¹⁸ is substituted C₁-C₆ alkyl, unsubstituted or substituted C₁-C₆ fluoroalkyl, or unsubstituted or substituted C₁-C₆ heteroalkyl. In some embodiments, R¹⁸ is C₁-C₆ alkyl which is substituted by —CN, —OH, or -Ome. In some embodiments, R¹⁸ is hydrogen.

In some embodiments, R¹⁸ is unsubstituted or substituted carbocycle or unsubstituted or substituted heterocycle. In some embodiments, R¹⁸ is unsubstituted or substituted C₃-C₁₀ cycloalkyl, unsubstituted or substituted 3- to 10-membered heterocycloalkyl, unsubstituted or substituted aryl, or unsubstituted or substituted heteroaryl. In some embodiments, R¹⁸ is unsubstituted or substituted C₃-C₆ monocyclic cycloalkyl, unsubstituted or substituted C₅-C₁₀ bicyclic cycloalkyl, or unsubstituted or substituted 3- to 10-membered monocyclic or bicyclic heterocycloalkyl. In some embodiments, R¹⁸ is unsubstituted or substituted 3- to 10-membered monocyclic or bicyclic heterocycloalkyl containing an oxygen atom; or R¹⁸ is unsubstituted or substituted 5- to 10-membered bicyclic heterocycloalkyl which is a bridged, fused, or spirocyclic heterocycloalkyl.

In some embodiments, R¹⁸ is —CHR²⁰-(unsubstituted or substituted carbocycle) or —CHR²⁰-(unsubstituted or substituted heterocycle). In some embodiments, R¹⁸ is —CHR²⁰-(unsubstituted or substituted C₃-C₆ cycloalkyl), —CHR²⁰-(unsubstituted or substituted 3- to 6-membered heterocycloalkyl), —CHR²⁰-(unsubstituted or substituted phenyl), or —CHR²⁰-(unsubstituted or substituted 5- or 6-membered heteroaryl); and R²⁰ is hydrogen or —CH₃.

In some embodiments, R¹⁸ is —C(═O)R¹⁹, —CO₂R¹⁹, or —C(═O)NHR¹⁹; and R¹⁹ is unsubstituted or substituted C₁-C₆ alkyl, unsubstituted or substituted C₃-C₁₀ cycloalkyl, unsubstituted or substituted 3- to 10-membered heterocycloalkyl.

In some embodiments, X² is CR¹⁷; and each R¹⁷ is hydrogen.

In some embodiments, R¹⁸ is hydrogen, unsubstituted or substituted C₁-C₆ alkyl, unsubstituted or substituted C₁-C₆ fluoroalkyl, unsubstituted or substituted C₁-C₆ heteroalkyl, unsubstituted or substituted C₁-C₆ alkenyl, unsubstituted or substituted C₁-C₆ alkynyl; or R¹⁸ is substituted C₁-C₆ alkyl, unsubstituted or substituted C₁-C₆ fluoroalkyl, unsubstituted or substituted C₁-C₆ heteroalkyl; or R¹⁸ is C₁-C₆ alkyl which is substituted by —CN, —OH, or -Ome; or R¹⁸ is unsubstituted or substituted C₃-C₁₀ cycloalkyl, unsubstituted or substituted 3- to 10-membered heterocycloalkyl, unsubstituted or substituted aryl, or unsubstituted or substituted heteroaryl; or R¹⁸ is unsubstituted or substituted C₃-C₆ monocyclic cycloalkyl, unsubstituted or substituted C₅-C₁₀ bicyclic cycloalkyl, or unsubstituted or substituted 3- to 10-membered monocyclic or bicyclic heterocycloalkyl; or R¹⁸ is unsubstituted or substituted 3- to 10-membered monocyclic or bicyclic heterocycloalkyl containing an oxygen atom; or R¹⁸ is unsubstituted or substituted 5- to 10-membered bicyclic heterocycloalkyl which is a bridged, fused, or spirocyclic heterocycloalkyl; or R^{1′} is —CHR²⁰-(unsubstituted or substituted C₃-C₆ cycloalkyl), —CHR²⁰-(unsubstituted or substituted 3- to 6-membered heterocycloalkyl), —CHR²⁰-(unsubstituted or substituted phenyl), or —CHR²⁰-(unsubstituted or substituted 5- or 6-membered heteroaryl); and R¹ is hydrogen or —CH₃; or R¹⁸ is —C(═O)R¹⁹, —CO₂R¹⁹, or —C(═O)NHR¹⁹; and R¹⁹ is unsubstituted or substituted C₁-C₆ alkyl, unsubstituted or substituted C₃-C₁₀ cycloalkyl, unsubstituted or substituted 3- to 10-membered heterocycloalkyl.

In some embodiments, described herein is a compound of Formula (VI):

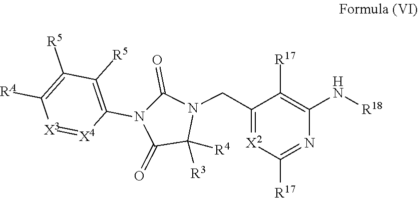

or a pharmaceutically acceptable salt or solvate thereof, wherein:
R^{A} is unsubstituted or substituted C₁-C₆ alkyl;
X³ and X⁴ are each independently CR⁵ or N;
R³ is hydrogen, unsubstituted or substituted C₁-C₆ alkyl, unsubstituted or substituted C₁-C₆ fluoroalkyl, unsubstituted or substituted C₁-C₆ alkoxy, unsubstituted or substituted phenyl, unsubstituted or substituted benzyl, unsubstituted or substituted monocyclic heteroaryl, unsubstituted or substituted C₃-C₆ cycloalkyl, or unsubstituted or substituted 3- to 6-membered heterocycloalkyl;
R⁴ is hydrogen or C₁-C₆ alkyl;
or R³ and R⁴ are taken together with the carbon atom to which they are attached to form a C₃-C₆ cycloalkyl or 3- to 6-membered heterocycloalkyl;
each R⁵ is independently hydrogen, halogen, unsubstituted or substituted C₁-C₆ alkyl, unsubstituted or substituted C₁-C₆ fluoroalkyl, —CN, —OH, —OR²¹, —CO₂R², —N(R²²)₂, —C(═O)N(R²²)₂, —NR²²C(═O)R²¹, —S(═O)₂N(R²¹)₂, or —NR²²S(═O)₂R²¹;
X² is CR¹⁷ or N;
each R¹⁷ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²¹, —CO₂R²², —N(R²²)₂, —C(═O)N(R²²)₂, —NR²²C(═O)R²¹, —S(═O)₂N(R²²)₂, or —NR²²S(═O)₂R²¹;
R¹⁸ is unsubstituted or substituted C₁-C₆ alkyl, unsubstituted or substituted C₁-C₆ fluoroalkyl, unsubstituted or substituted C₁-C₆ heteroalkyl, unsubstituted or substituted C₁-C₆ alkenyl, or unsubstituted or substituted C₁-C₆ alkynyl;
or R¹⁸ is substituted cyclopropyl, unsubstituted or substituted cyclobutyl, unsubstituted or substituted cyclopentyl, or unsubstituted or substituted cyclohexyl;
or R¹⁸ is unsubstituted or substituted C₅-C₁₀ bicyclic cycloalkyl;
or R¹⁸ is unsubstituted or substituted 3- to 10-membered monocyclic or bicyclic heterocycloalkyl containing an oxygen atom;
or R¹⁸ is —CHR²⁰-(unsubstituted or substituted carbocycle) or —CHR²⁰-(unsubstituted or substituted heterocycle);
   R²⁰ is hydrogen or —CH₃;
each R²¹ is independently unsubstituted or substituted C₁-C₆ alkyl, unsubstituted or substituted C₃-C₆ cycloalkyl, unsubstituted or substituted 3- to 6-membered heterocycloalkyl, unsubstituted or substituted aryl, or unsubstituted or substituted heteroaryl;
each R²² is independently hydrogen, unsubstituted or substituted C₁-C₆ alkyl, unsubstituted or substituted C₃-C₆ cycloalkyl, unsubstituted or substituted 3- to 6-membered heterocycloalkyl, unsubstituted or substituted aryl, or unsubstituted or substituted heteroaryl; or two R²² on the same N atom are taken together with the N atom to which they are attached to form an unsubstituted or substituted N-containing heterocycloalkyl;
wherein each substituted alkyl, substituted fluoroalkyl, substituted aryl, substituted heteroaryl, substituted cycloalkyl, and substituted heterocycloalkyl is substituted with one or more R^{s} groups independently selected from the group consisting of halogen, C₁-C₆ alkyl, C₁-C₆ fluoroalkyl, C₃-C₆ cycloalkyl, 3- to 6-membered heterocycloalkyl, phenyl, benzyl, 5-membered heteroaryl, 6-membered heteroaryl, —CN, —OR²³, —CO₂R²³—, —C(═O)N(R²³)₂, —N(R²³)₂, —NR²³C(═O)R²⁴, —SR²³, —S(═O)R²⁴, —SO₂R²⁴, or —SO₂N(R²³)₂;
   each R²³ is independently selected from hydrogen, C₁-C₆ alkyl, C₃-C₆ cycloalkyl, 3- to 6-membered heterocycloalkyl, phenyl, benzyl, 5-membered heteroaryl and 6-membered heteroaryl; or two R²³ groups are taken together with the N atom to which they are attached to form a 3- to 6-membered N-containing heterocycloalkyl;
   each R²⁴ is independently selected from C₁-C₆ alkyl, C₃-C₆ cycloalkyl, 3- to 6-membered heterocycloalkyl, phenyl, benzyl, 5-membered heteroaryl and 6-membered heteroaryl.

### Additional Embodiments of Formula (V)

In some embodiments, R³ is hydrogen, unsubstituted or substituted C₁-C₆ alkyl, unsubstituted or substituted C₁-C₆ fluoroalkyl, unsubstituted or substituted C₁-C₆ alkoxy, unsubstituted or substituted phenyl, unsubstituted or substituted benzyl, unsubstituted or substituted monocyclic heteroaryl, unsubstituted or substituted C₃-C₆ cycloalkyl, or unsubstituted or substituted 3- to 6-membered heterocycloalkyl. In some embodiments, R³ is hydrogen, unsubstituted or substituted C₁-C₆ alkyl, unsubstituted or substituted C₁-C₆ fluoroalkyl, unsubstituted or substituted phenyl, unsubstituted or substituted benzyl, or unsubstituted or substituted C₃-C₆ cycloalkyl. In some embodiments, R³ is unsubstituted or substituted C₁-C₆ alkyl, phenyl, benzyl, or C₃-C₆ cycloalkyl. In some embodiments, R³ is C₁—C alkyl which is unsubstituted or substituted by —OH, phenyl, benzyl, or C₃-C₆ cycloalkyl. In some embodiments, R³ is methyl, ethyl, propyl, isopropyl, butyl, isobutyl, sec-butyl, phenyl, benzyl, cyclopropyl, cyclobutyl, cyclopentyl, cyclohexyl, or —CH₂OH

In some embodiments, R⁴ is hydrogen or C₁-C₆ alkyl. In some embodiments, R⁴ is hydrogen or C₁-C₄ alkyl. In some embodiments, R⁴ is hydrogen or methyl.

In some embodiments, R³ and R⁴ are taken together with the carbon atom to which they are attached to form a C₃-C₆ cycloalkyl or 3- to 6-membered heterocycloalkyl. In some embodiments, R³ and R⁴ are taken together with the carbon atom to which they are attached to form a C₃-C₆ cycloalkyl. In some embodiments, R³ and R⁴ are taken together with the carbon atom to which they are attached to form a cyclopropyl, cyclobutyl, cyclopentyl, or cyclohexyl.

In some embodiments, R³ is C₁-C₆ alkyl; and R⁴ is C₁-C₆ alkyl; or R³ and R⁴ are taken together with the carbon atom to which they are attached to form a C₃-C₆ cycloalkyl.

In some embodiments, each R⁵ is independently hydrogen, halogen, unsubstituted or substituted C₁-C₆ alkyl, unsubstituted or substituted C₁-C₆ fluoroalkyl, —CN, —OH, —OR²¹, —CO₂R²², —N(R²²)₂, —C(═O)N(R²²)₂, —NR²²C(═O)R²¹, —S(═O)₂N(R²²)₂, or —NR²²S(═O)₂R²¹. In some embodiments, each R⁵ is independently hydrogen, halogen, C₁-C₆ alkyl, C₁-C₆ fluoroalkyl, C₃-C₆ cycloalkyl, 3- to 6-membered heterocycloalkyl, —CN, or —OR¹. In some embodiments, each R⁵ is independently hydrogen or C₁-C₆ alkyl. In some embodiments, each R⁵ is hydrogen.

In some embodiments, X³ and X⁴ are each CR⁵; and each R⁵ is hydrogen.

In some embodiments, R^{A} is C₁-C₄ alkyl. In some embodiments, R^{A} is iso-propyl or t-butyl. In some embodiments, R^{A} is iso-propyl. In some embodiments, R^{A} is t-butyl.

In some embodiments, X² is CR¹⁷ or N. In some embodiments, X² is CR¹⁷. In some embodiments. X² is N.

In some embodiments, each R¹⁷ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²¹, —CO₂R²², —N(R²²)₂, —C(═O)N(R²²)₂, —OC(═O)R²¹, —NR²²C(═O)R²¹—S(═O)₂N(R²²)₂, or —NR²²S(═O)₂R²¹. In some embodiments, each R¹⁷ is independently hydrogen, halogen, unsubstituted or substituted C₁-C₆ alkyl, unsubstituted or substituted C₁-C₆ fluoroalkyl, —CN, —OH, —OR²¹, —CO₂R²², —N(R²²)₂, —C(═O)N(R²²)₂, —OC(═O)R²¹, —NR²²C(═O)R²¹, —S(═O)₂N(R²²)₂, or —NR²S(═O)₂R²¹. In some embodiments, each R¹⁷ is independently hydrogen, halogen, C₁-C₆ alkyl, C₁-C₆ fluoroalkyl, —CN, —OH, —OR²¹, —N(R²²)₂, or —C(═O)N(R²²)₂. In some embodiments, each R¹⁷ is independently hydrogen, halogen, C₁-C₆ alkyl, or C₁-C₆ fluoroalkyl. In some embodiments, each R¹⁷ is hydrogen.

In some embodiments, X² is CR¹⁷; and each R¹⁷ is independently hydrogen, halogen, C₁-C₆ alkyl, or C₁-C₆ fluoroalkyl.

In some embodiments, R¹⁸ is unsubstituted or substituted C₁-C₆ alkyl, unsubstituted or substituted C₁-C₆ fluoroalkyl, unsubstituted or substituted C₁-C₆ heteroalkyl, unsubstituted or substituted C₁-C₆ alkenyl, or unsubstituted or substituted C₁-C₆ alkynyl. In some embodiments, R¹⁸ is unsubstituted or substituted C₁-C₆ alkyl, unsubstituted or substituted C₁-C₆ fluoroalkyl, unsubstituted or substituted C₁-C₆ heteroalkyl, unsubstituted or substituted C₁-C₆ alkenyl, unsubstituted or substituted C₁-C₆ alkynyl. In some embodiments, R¹⁸ is substituted C₁-C₆ alkyl, unsubstituted or substituted C₁-C₆ fluoroalkyl, or unsubstituted or substituted C₁-C₆ heteroalkyl. In some embodiments, R¹⁸ is C₁-C₆ alkyl which is substituted by —CN, —OH, or -Ome.

In some embodiments, R¹⁸ is substituted cyclopropyl, unsubstituted or substituted cyclobutyl, unsubstituted or substituted cyclopentyl, or unsubstituted or substituted cyclohexyl.

In some embodiments, R¹⁸ is unsubstituted or substituted C₅-C₁₀ bicyclic cycloalkyl. In some embodiments, R¹⁸ is C₅-C₁₀ cycloalkyl which is a bridged, fused, or spirocyclic cycloalkyl.

In some embodiments, R¹⁸ is unsubstituted or substituted 3- to 10-membered monocyclic or bicyclic heterocycloalkyl containing an oxygen atom. In some embodiments, R¹⁸ is unsubstituted or substituted 3- to 10-membered monocyclic or bicyclic heterocycloalkyl containing an oxygen atom which is a bridged, fused, or spirocyclic heterocycloalkyl.

In some embodiments, R¹⁸ is —CHR²⁰-(unsubstituted or substituted carbocycle) or —CHR²⁰-(unsubstituted or substituted heterocycle). In some embodiments, R¹⁸ is —CHR²⁰-(unsubstituted or substituted C₃-C₆ cycloalkyl), —CHR²⁰-(unsubstituted or substituted 3- to 6-membered heterocycloalkyl), —CHR²⁰-(unsubstituted or substituted phenyl), or —CHR²⁰-(unsubstituted or substituted 5- or 6-membered heteroaryl); and R²⁰ is hydrogen or —CH₃.

In some embodiments, described herein is a compound of Formula (VII):

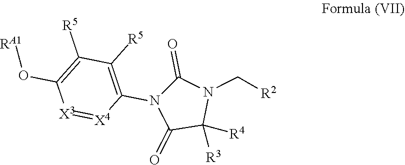

or a pharmaceutically acceptable salt or solvate thereof, wherein:
R^{A1} is C₁-C₆ alkyl, C₁-C₆ fluoroalkyl, C₃-C₆ cycloalkyl, or 3- to 6-membered heterocycloalkyl;
X³ and X⁴ are each independently CR⁵ or N;
R² is

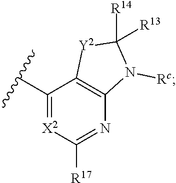

R^{c} is hydrogen, C₁-C₆ alkyl, C₁-C₆ fluoroalkyl, —C(═O)N(R²²)₂, —C(═O)R²¹, —C(═O)OR²², or —S(═O)₂R²¹;
X² is CR¹⁷ or N;
each R¹⁷ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²¹, —CO₂R²², —N(R²²)₂, —C(═O)N(R²²)₂, —OC(═O)R²¹, —NR²²C(═O)R²¹, —S(═O)₂N(R²²)₂, or —NR²²S(═O)₂R²¹;
R¹³ and R¹⁴ are each independently hydrogen, halogen, unsubstituted or substituted C₁-C₆ alkyl, C₁-C₆ fluoroalkyl, phenyl, monocyclic heteroaryl, C₃-C₆ cycloalkyl, 3- to 6-membered heterocycloalkyl, —CN, —OH, —OR²¹, —CO₂R²², —N(R²²)₂, —C(═O)N(R²²)₂, or —OC(═O)R²¹;
or R¹³ and R¹⁴ are taken together with the carbon atom to which they are attached to form a —C(═O)—;
or R¹³ and R¹⁴ are taken together with the carbon atom to which they are attached to form a C₃-C₆ cycloalkyl or 3- to 6-membered heterocycloalkyl;
Y² is —(CR¹⁵R¹⁶)ₙ—, —O—, or —O—CR¹⁵R¹⁶—;
   each R¹⁵ and R¹⁶ is independently hydrogen, halogen, cyano, C₁-C₆ alkyl, C₁-C₆ fluoroalkyl, C₁-C₆ alkoxy, phenyl, monocyclic heteroaryl, C₃-C₆ cycloalkyl, or 3- to 6-membered heterocycloalkyl;
   or R⁴ and one R¹⁶ on an adjacent carbon atom are taken together to form an alkene bond;
   n is 1 or 2;
or R² is

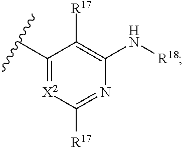

X² is CR¹⁷ or N;
each R¹⁷ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²¹, —CO₂R²², —N(R²²)₂, —C(═O)N(R²²)₂, —NR²²C(═O)R²¹, —S(═O)₂N(R²²)₂, or —NR²²S(═O)₂R²¹;
R¹⁸ is substituted C₁-C₆ alkyl, unsubstituted or substituted C₁-C₆ fluoroalkyl, unsubstituted or substituted C₁-C₆ heteroalkyl, unsubstituted or substituted C₁-C₆ alkenyl, or unsubstituted or substituted C₁-C₆ alkynyl;
or R¹⁸ is unsubstituted or substituted cyclopropyl, unsubstituted or substituted cyclobutyl, unsubstituted or substituted cyclopentyl, or substituted cyclohexyl;
or R¹⁸ is unsubstituted or substituted C₅-C₁₀ bicyclic cycloalkyl;
or R¹⁸ is unsubstituted or substituted 3- to 10-membered monocyclic or bicyclic heterocycloalkyl;
or R¹⁸ is —CHR²⁰-(unsubstituted or substituted carbocycle) or —CHR²⁰-(unsubstituted or substituted heterocycle);
   R²⁰ is hydrogen or —CH₃;
R³ is hydrogen, unsubstituted or substituted C₁-C₆ alkyl, unsubstituted or substituted C₁-C₆ fluoroalkyl, unsubstituted or substituted C₁-C₆ alkoxy, unsubstituted or substituted phenyl, unsubstituted or substituted benzyl, unsubstituted or substituted monocyclic heteroaryl, unsubstituted or substituted C₃-C₆ cycloalkyl, or unsubstituted or substituted 3- to 6-membered heterocycloalkyl;
R⁴ is hydrogen or C₁-C₆ alkyl;
or R³ and R⁴ are taken together with the carbon atom to which they are attached to form a C₃-C₆ cycloalkyl or 3- to 6-membered heterocycloalkyl;
each R⁵ is independently hydrogen, halogen, unsubstituted or substituted C₁-C₆ alkyl, unsubstituted or substituted C₁-C₆ fluoroalkyl, —CN, —OH, —OR²¹, —CO₂R²², —N(R²²)₂, —C(═O)N(R²²)₂, —NR²²C(═O)R²¹, —S(═O)₂N(R²²)₂, or —NR²²S(═O)₂R²¹;
each R²¹ is independently unsubstituted or substituted C₁-C₆ alkyl, unsubstituted or substituted C₃-C₆ cycloalkyl, unsubstituted or substituted 3- to 6-membered heterocycloalkyl, unsubstituted or substituted aryl, or unsubstituted or substituted heteroaryl;
each R²² is independently hydrogen, unsubstituted or substituted C₁-C₆ alkyl, unsubstituted or substituted C₃-C₆ cycloalkyl, unsubstituted or substituted 3- to 6-membered heterocycloalkyl, unsubstituted or substituted aryl, or unsubstituted or substituted heteroaryl; or two R²² on the same N atom are taken together with the N atom to which they are attached to form an unsubstituted or substituted N-containing heterocycloalkyl;
wherein each substituted alkyl, substituted fluoroalkyl, substituted aryl, substituted heteroaryl, substituted cycloalkyl, and substituted heterocycloalkyl is substituted with one or more R^{s} groups independently selected from the group consisting of halogen, C₁-C₆ alkyl, C₁-C₆ fluoroalkyl, C₃-C₆ cycloalkyl, 3- to 6-membered heterocycloalkyl, phenyl, benzyl, 5-membered heteroaryl, 6-membered heteroaryl, —CN, —OR²³, —CO₂R²³, —C(═O)N(R²³)₂, —N(R²³)₂, —NR²³C(═O)R²⁴, —SR²³, —S(═O)R²⁴, —SO₂R²⁴, or —SO₂N(R²³)₂;
   each R²³ is independently selected from hydrogen, C₁-C₆ alkyl, C₃-C₆ cycloalkyl, 3- to 6-membered heterocycloalkyl, phenyl, benzyl, 5-membered heteroaryl and 6-membered heteroaryl; or two R²³ groups are taken together with the N atom to which they are attached to form a 3- to 6-membered N-containing heterocycloalkyl;
      each R²⁴ is independently selected from C₁-C₆ alkyl, C₃-C₆ cycloalkyl, 3- to 6-membered heterocycloalkyl, phenyl, benzyl, 5-membered heteroaryl and 6-membered heteroaryl.

### Additional Embodiments of Formula (VII)

In some embodiments, R³ is hydrogen, unsubstituted or substituted C₁-C₆ alkyl, unsubstituted or substituted C₁-C₆ fluoroalkyl, unsubstituted or substituted C₁-C₆ alkoxy, unsubstituted or substituted phenyl, unsubstituted or substituted benzyl, unsubstituted or substituted monocyclic heteroaryl, unsubstituted or substituted C₃-C₆ cycloalkyl, or unsubstituted or substituted 3- to 6-membered heterocycloalkyl. In some embodiments, R³ is hydrogen, unsubstituted or substituted C₁-C₆ alkyl, unsubstituted or substituted C₁-C₆ fluoroalkyl, unsubstituted or substituted phenyl, unsubstituted or substituted benzyl, or unsubstituted or substituted C₃-C₆ cycloalkyl. In some embodiments, R³ is unsubstituted or substituted C₁-C₆ alkyl, phenyl, benzyl, or C₃-C₆ cycloalkyl. In some embodiments, R³ is C₁-C₆ alkyl which is unsubstituted or substituted by —OH, phenyl, benzyl, or C₃-C₆ cycloalkyl. In some embodiments, R³ is methyl, ethyl, propyl, isopropyl, butyl, isobutyl, sec-butyl, phenyl, benzyl, cyclopropyl, cyclobutyl, cyclopentyl, cyclohexyl, or —CH₂OH

In some embodiments, R⁴ is hydrogen or C₁-C₆ alkyl. In some embodiments, R⁴ is hydrogen or C₁-C₄ alkyl. In some embodiments, R⁴ is hydrogen or methyl.

In some embodiments, R³ and R⁴ are taken together with the carbon atom to which they are attached to form a C₃-C₆ cycloalkyl or 3- to 6-membered heterocycloalkyl. In some embodiments, R³ and R⁴ are taken together with the carbon atom to which they are attached to form a C₃-C₆ cycloalkyl. In some embodiments, R³ and R⁴ are taken together with the carbon atom to which they are attached to form a cyclopropyl, cyclobutyl, cyclopentyl, or cyclohexyl.

In some embodiments, R³ is C₁-C₆ alkyl; and R⁴ is C₁-C₆ alkyl; or R³ and R⁴ are taken together with the carbon atom to which they are attached to form a C₃-C₆ cycloalkyl.

In some embodiments, each R⁵ is independently hydrogen, halogen, unsubstituted or substituted C₁-C₆ alkyl, unsubstituted or substituted C₁-C₆ fluoroalkyl, —CN, —OH, —OR²¹, —CO₂R²², —N(R²²)₂, —C(═O)N(R²²)₂, —NR²²C(═O)R²¹, —S(═O)₂N(R²²)₂, or —NR²²S(═O)₂R²². In some embodiments, each R⁵ is independently hydrogen, halogen, C₁-C₆ alkyl, C₁-C₆ fluoroalkyl, C₃-C₆ cycloalkyl, 3- to 6-membered heterocycloalkyl, —CN, or —OR²¹. In some embodiments, each R⁵ is independently hydrogen or C₁-C₆ alkyl. In some embodiments, each R⁵ is hydrogen.

In some embodiments, X³ and X⁴ are each CR⁵; and each R⁵ is hydrogen.

In some embodiments, R^{A} is C₁-C₄ alkyl or C₁-C₄ fluoroalkyl. In some embodiments, R^{A1} is C₁-C₄ fluoroalkyl. In some embodiments, R^{A1} is —CF₃.

In some embodiments, R² is

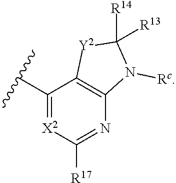

In some embodiments, R is hydrogen, C₁-C₆ alkyl, C₁-C₆ fluoroalkyl, —C(═O)N(R²²)₂, —C(═O)R²¹, —C(═O)OR²², or —S(═O)₂R²¹. In some embodiments. R is hydrogen, C₁-C₆ alkyl, C₁-C₆ fluoroalkyl, —C(═O)N(R²²)₂, —C(═O)R²¹, or —C(═O)OR²². In some embodiments, R^{c} is hydrogen, C₁-C₆ alkyl, C₁-C₆ fluoroalkyl, or —C(═O)OR². In some embodiments, R^{c} is hydrogen, C₁-C₆ alkyl, or —C(═O)OR²². In some embodiments, R^{c} is hydrogen, C₁-C₆ alkyl, or C₁-C₆ fluoroalkyl. In some embodiments, R is hydrogen or C₁-C₆ alkyl. In some embodiments, R^{c} is hydrogen.

In some embodiments, X² is CR¹⁷ or N. In some embodiments, X² is CR¹⁷. In some embodiments, X² is N.

In some embodiments, Y² is —(CR¹⁵R¹⁶)ₙ—, —O—, or —O—CR¹⁵R¹⁶—. In some embodiments, Y² is —(CR¹⁵R¹⁶)ₙ—, and n is 1 or 2. In some embodiments, Y² is —(CR¹⁵R¹⁶)— or —(CR¹⁵R¹⁶)—(CR¹⁵R¹⁶)—. In some embodiments, Y² is —O—. In some embodiments, Y² is —O—CR¹⁵R¹⁶—.

In some embodiments, R¹³ and R¹⁴ are each independently hydrogen, halogen, unsubstituted or substituted C₁-C₆ alkyl, C₁-C₆ fluoroalkyl, phenyl, monocyclic heteroaryl, C₃-C₆ cycloalkyl, 3- to 6-membered heterocycloalkyl, —CN, —OH, —OR²¹, —CO₂R²², —N(R²²)₂, —C(═O)N(R²²)₂, or —OC(═O)R²¹. In some embodiments, R¹³ and R¹⁴ are each independently hydrogen, halogen, C₁-C₆ alkyl, C₁-C₆ fluoroalkyl, C₃-C₆ cycloalkyl, or 3- to 6-membered heterocycloalkyl. In some embodiments, R¹³ and R¹⁴ are each independently hydrogen or C₁-C₆ alkyl. In some embodiments, R¹³ and R¹⁴ are each hydrogen.

In some embodiments, R¹³ and R¹⁴ are taken together with the carbon atom to which they are attached to form a —C(═O)—.

In some embodiments, R¹³ and R¹⁴ are taken together with the carbon atom to which they are attached to form a C₃-C₆ cycloalkyl or 3- to 6-membered heterocycloalkyl. In some embodiments, R¹³ and R¹⁴ are taken together with the carbon atom to which they are attached to form a C₃-C₆ cycloalkyl. In some embodiments, R¹³ and R¹⁴ are taken together with the carbon atom to which they are attached to form a C₃-C₄ cycloalkyl. In some embodiments, R¹³ and R¹⁴ are taken together with the carbon atom to which they are attached to form a 3- to 6-membered heterocycloalkyl. In some embodiments, R³ and R¹⁴ are taken together with the carbon atom to which they are attached to form a 3- or 4-membered heterocycloalkyl.

In some embodiments, each R¹⁵ and R¹⁶ is independently hydrogen, halogen, cyano, C₁-C₆ alkyl, C₁-C₆ fluoroalkyl, C₁-C₆ alkoxy, phenyl, monocyclic heteroaryl, C₃-C₆ cycloalkyl, or 3- to 6-membered heterocycloalkyl. In some embodiments, each R¹⁵ and R¹⁶ is independently hydrogen, halogen, C₁-C₆ alkyl, C₁-C₆ fluoroalkyl, or C₃-C₆ cycloalkyl. In some embodiments, each R¹⁵ and R¹⁶ is independently hydrogen, C₁-C₆ alkyl, C₁-C₆ fluoroalkyl, or C₃-C₆ cycloalkyl. In some embodiments, each R¹⁵ and R¹⁶ is independently hydrogen or C₁-C₆ alkyl. In some embodiments, each R¹⁵ and R¹⁶ is independently hydrogen.

In some embodiments, R¹⁴ and one R¹⁶ on an adjacent carbon atom are taken together to form an alkene bond.

In some embodiments, each R¹⁷ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²¹, —CO₂R²², —N(R²²)₂, —C(═O)N(R²², —OC(═O)R²¹, —NR²²C(═O)R²¹, —S(═O)₂N(R²²)₂, or —NR²S(═O)₂R²¹. In some embodiments, each R¹⁷ is independently hydrogen, halogen, unsubstituted or substituted C₁-C₆ alkyl, unsubstituted or substituted C₁-C₆ fluoroalkyl, —CN, —OH, —OR²¹, —CO₂R²², —N(R²²)₂, —C(═O)N(R²²)₂, —OC(═O)R²¹, —NR²²C(═O)R²¹, —S(═O)₂N(R²²)₂, or —NR²²S(═O)₂R²¹. In some embodiments, each R¹⁷ is independently hydrogen, halogen, C₁-C₆ alkyl, C₁-C₆ fluoroalkyl. —CN, —OH, —OR²¹, —N(R²²)₂, or —C(═O)N(R²²)₂. In some embodiments, each R⁷ is independently hydrogen, halogen, C₁-C₆ alkyl, or C₁-C₆ fluoroalkyl. In some embodiments, each R¹⁷ is hydrogen.

In some embodiments, R^{c} is hydrogen, C₁-C₆ alkyl, or —C(═O)OR²²; X² is CR¹⁷; each R¹⁷ is independently hydrogen, halogen, C₁-C₆ alkyl, or C₁-C₆ fluoroalkyl; R¹³ and R¹⁴ are each independently hydrogen, halogen, C₁-C₆ alkyl, C₁-C₆ fluoroalkyl, C₃-C₆ cycloalkyl, or 3- to 6-membered heterocycloalkyl; or R¹³ and R¹⁴ are taken together with the carbon atom to which they are attached to form a —C(═O)—; or R¹³ and R¹⁴ are taken together with the carbon atom to which they are attached to form a C₃-C₄ cycloalkyl; Y² is —(CR¹⁵R¹⁶)ₙ—, —O—, or —O—CR¹⁵R¹⁶—; and each R¹⁵ and R¹⁶ is independently hydrogen, halogen, C₁-C₆ alkyl, C₁-C₆ fluoroalkyl, or C₃-C₆ cycloalkyl; or R¹⁴ and one R¹⁶ on an adjacent carbon atom are taken together to form an alkene bond.

In some embodiments, R^{c} is hydrogen; X² is CR¹⁷; each R¹⁷ is hydrogen; R¹³ and R¹⁴ are each hydrogen; or R³ and R¹⁴ are taken together with the carbon atom to which they are attached to form a —C(═O)—; and Y² is —(CR¹⁵R¹⁶). —O—, or —O—CR¹⁵R¹⁶—. In some embodiments, each R¹⁵ and R¹⁶ is independently hydrogen, C₁-C₆ alkyl, C₁-C₆ fluoroalkyl, or C₃-C₆ cycloalkyl.

In some embodiments,

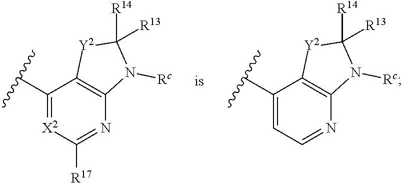

and Y² is —CR¹⁵R¹⁶— or —CR¹⁵R¹⁶—CR¹⁵R¹⁶—. In some embodiments,

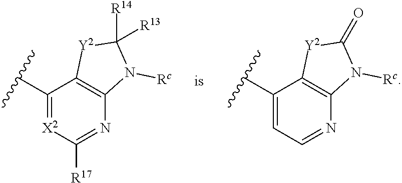

In some embodiments, R¹⁴ and one R¹⁶ on an adjacent carbon atom are taken together to form an alkene bond.

In some embodiments, R² is

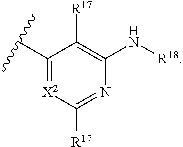

In some embodiments, X² is CR¹⁷ or N. In some embodiments, X² is CR¹⁷. In some embodiments, X² is N.

In some embodiments, each R¹⁷ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²¹, —CO₂R²², —N(R²²)₂, —C(═O)N(R²²)₂—OC(═O)R²¹, —NR²²C(═O)R²¹, —S(═O)₂N(R²²)₂, or —NR²²S(═O)₂R²¹. In some embodiments, each R¹⁷ is independently hydrogen, halogen, unsubstituted or substituted C₁-C₆ alkyl, unsubstituted or substituted C₁—C fluoroalkyl, —CN, —OH, —OR²¹, —CO₂R²², —N(R²²)₂, —C(═O)N(R²²)₂, —OC(═O)R²¹, —NR²²C(═O)R²¹, —S(═O)₂N(R²²)₂, or —NR²²S(═O)₂R²¹. In some embodiments, each R¹⁷ is independently hydrogen, halogen, C₁-C₆ alkyl, C₁-C₆ fluoroalkyl, —CN, —OH, —OR²¹, —N(R²²)₂, or —C(═O)N(R²²)₂. In some embodiments, each R⁷ is independently hydrogen, halogen, C₁-C₆ alkyl, or C₁-C₆ fluoroalkyl. In some embodiments, each R¹⁷ is hydrogen.

In some embodiments, X² is CR¹⁷; and each R¹⁷ is independently hydrogen, halogen, C₁-C₆ alkyl, or C₁-C₆ fluoroalkyl.

In some embodiments, R¹⁸ is substituted C₁-C₆ alkyl, unsubstituted or substituted C₁-C₆ fluoroalkyl, unsubstituted or substituted C₁-C₆ heteroalkyl, unsubstituted or substituted C₁-C₆ alkenyl, unsubstituted or substituted C₁-C₆ alkynyl. In some embodiments, R¹⁸ is substituted C₁-C₆ alkyl, unsubstituted or substituted C₁-C₆ fluoroalkyl, unsubstituted or substituted C₁-C₆ heteroalkyl. In some embodiments, R¹⁸ is C₁-C₆ alkyl which is substituted by —CN, —OH, or -Ome.

In some embodiments, R¹⁸ is unsubstituted or substituted cyclopropyl, unsubstituted or substituted cyclobutyl, unsubstituted or substituted cyclopentyl, or substituted cyclohexyl.

In some embodiments, R¹⁸ is unsubstituted or substituted C₅-C₁₀ bicyclic cycloalkyl which is a bridged, fused, or spirocyclic heterocycloalkyl.

In some embodiments, R¹⁸ is unsubstituted or substituted 3- to 10-membered monocyclic or bicyclic heterocycloalkyl.

In some embodiments, R¹⁸ is unsubstituted or substituted 3- to 10-membered monocyclic or bicyclic heterocycloalkyl containing an oxygen atom:

In some embodiments, R¹⁸ is unsubstituted or substituted 5- to 10-membered bicyclic heterocycloalkyl which is a bridged, fused, or spirocyclic heterocycloalkyl.

In some embodiments, R¹⁸ is —CHR²⁰-(unsubstituted or substituted C₃-C₆ cycloalkyl), —CHR²⁰-(unsubstituted or substituted 3- to 6-membered heterocycloalkyl), —CHR²⁰-(unsubstituted or substituted phenyl), or —CHR²⁰-(unsubstituted or substituted 5- or 6-membered heteroaryl); and R²⁰ is hydrogen or —CH₃.

In some embodiments, described herein is a compound of Formula (VII):

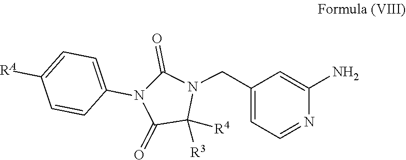

or a pharmaceutically acceptable salt or solvate thereof, wherein:
R¹ is —SCF₃, —S(═O)CF₃, or —S(═O)₂CF₃;
R³ is C₂-C₆ alkyl which is unsubstituted or substituted by —OH, phenyl, benzyl, C₃-C₆ cycloalkyl, or —CH₂OH; and
R⁴ is hydrogen or methyl;
or R³ and R⁴ are taken together with the carbon atom to which they are attached to form a cyclobutyl, cyclopentyl, or cyclohexyl.

### Additional Embodiments of Formula (VIII)

In some embodiments, R^{A} is —SCF₃ or —S(═O)₂CF₃. In some embodiments, R^{A} is —SCF₃. In some embodiments, R^{A} is —S(═O)CF₃. In some embodiments, R^{A} is —S(═O)₂CF₃.

In some embodiments, R³ is C₂-C₆ alkyl which is unsubstituted or substituted by —OH, phenyl, benzyl, C₃-C₆ cycloalkyl, or —CH₂OH; and R⁴ is hydrogen or methyl. In some embodiments, R³ is ethyl, propyl, isopropyl, butyl, isobutyl, sec-butyl, phenyl, benzyl, cyclopropyl, cyclobutyl, cyclopentyl, cyclohexyl, or —CH₂OH; and R⁴ is hydrogen or methyl.

In some embodiments, R³ and R⁴ are taken together with the carbon atom to which they are attached to form a cyclobutyl, cyclopentyl, or cyclohexyl.

In some embodiments, described herein is a compound of Formula (IX):

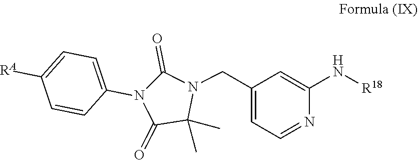

or a pharmaceutically acceptable salt or solvate thereof, wherein:
R^{A1} is —SR^{A1}, —S(═O)R^{A1}, or —S(═O)₂R^{A1};
   R^{A1} is C₁-C₄ alkyl, C₁-C₄ fluoroalkyl, or C₃-C₆ cycloalkyl:
R¹⁸ is C₁-C₆ alkyl which is substituted by —CN, —OH, or -Ome;
or R¹⁸ is unsubstituted or substituted C₁-C₆ fluoroalkyl or unsubstituted or substituted C₁-C₆ heteroalkyl;
or R¹⁸ is unsubstituted or substituted 3- to 10-membered monocyclic or bicyclic heterocycloalkyl;
or R¹⁸ is —CHR²⁰-(unsubstituted or substituted C₃-C₆ cycloalkyl), —CHR²⁰-(unsubstituted or substituted 3- to 6-membered heterocycloalkyl), or —CHR²⁰-(unsubstituted or substituted 5-membered heteroaryl);
   R²⁰ is hydrogen or —CH₃;
or R¹⁸ is —C(═O)R¹⁹, where R¹⁹ is unsubstituted or substituted 3- to 10-membered heterocycloalkyl;
or R¹⁸ is —C(═O)NHR¹⁹, where R¹⁹ is unsubstituted or substituted C₃-C₁₀ cycloalkyl or unsubstituted or substituted 3- to 10-membered heterocycloalkyl;
wherein each substituted alkyl, substituted fluoroalkyl, substituted aryl, substituted heteroaryl, substituted cycloalkyl, and substituted heterocycloalkyl is substituted with one or more R^{s} groups independently selected from the group consisting of halogen, C₁-C₆ alkyl, C₁-C₆ fluoroalkyl, C₃-C₆ cycloalkyl, 3- to 6-membered heterocycloalkyl, phenyl, benzyl, 5-membered heteroaryl, 6-membered heteroaryl. —CN, —OR²³, —CO₂R²³, —C(═O)N(R²³)₂, —N(R²³)₂, —NR²³C(═O)R²⁴, —SR²³, —S(═O)R²⁴, —SO₂R²⁴, or —SO₂N(R²³)₂;
   each R²³ is independently selected from hydrogen. C₁-C₆ alkyl, C₃-C₆ cycloalkyl, 3- to 6-membered heterocycloalkyl, phenyl, benzyl, 5-membered heteroaryl and 6-membered heteroaryl; or two R³ groups are taken together with the N atom to which they are attached to form a 3- to 6-membered N-containing heterocycloalkyl;
each R²⁴ is independently selected from C₁-C₆ alkyl, C₃-C₆ cycloalkyl, 3- to 6-membered heterocycloalkyl, phenyl, benzyl, 5-membered heteroaryl and 6-membered heteroaryl.

### Additional Embodiments of Formula (IX)

In some embodiments, R^{A} is —SR^{A1} or —S(═O)R^{A1}. In some embodiments, R^{A} is —SR^{A1}. In some embodiments, R^{A} is —S(═O)R^{A1}. In some embodiments, R^{A} is —S(═O)₂R^{A1}.

In some embodiments, R^{A1} is C₁-C₄ alkyl, C₁-C₄ fluoroalkyl, or C₃-C₆ cycloalkyl. In some embodiments, R^{A1} is C₁-C₄ alkyl or C₁-C₄ fluoroalkyl. In some embodiments, R^{A1} is C₁-C₄ fluoroalkyl. In some embodiments, R^{A1} is —CF₃, —CHF₂, —C(CH₃)₃, or -(cyclopropyl).

In some embodiments, R^{A} is —SCF₃, —S(═O)CF₃, —S(═O)₂CF₃, —S(═O)₂CHF₂, —S(═O)₂C(CH₃)₃, or —S(═O)₂-(cyclopropyl).

In some embodiments, R¹⁸ is C₁-C₆ alkyl which is substituted by —CN, —OH, or -Ome.

In some embodiments, R¹⁸ is unsubstituted or substituted C₁-C₆ fluoroalkyl or unsubstituted or substituted C₁-C₆ heteroalkyl.

In some embodiments, R¹⁸ is unsubstituted or substituted 3- to 10-membered monocyclic or bicyclic heterocycloalkyl.

In some embodiments, R¹⁸ is unsubstituted or substituted 3- to 10-membered monocyclic or bicyclic heterocycloalkyl containing an oxygen atom:

In some embodiments, R¹⁸ is unsubstituted or substituted 5- to 10-membered bicyclic heterocycloalkyl which is a bridged, fused, or spirocyclic heterocycloalkyl.

In some embodiments, R¹⁸ is —CHR²-(unsubstituted or substituted C₃-C₆ cycloalkyl), —CHR²⁰-(unsubstituted or substituted 3- to 6-membered heterocycloalkyl), or —CHR²¹-(unsubstituted or substituted 5-membered heteroaryl); and R²⁰ is hydrogen or —CH₃.

In some embodiments, R¹⁸ is —C(═O)R¹⁹, where R¹⁹ is unsubstituted or substituted 3- to 10-membered heterocycloalkyl.

In some embodiments, R¹⁸ is —C(═O)NHR¹⁹, where R¹⁹ is unsubstituted or substituted C₃-C₁₀ cycloalkyl or unsubstituted or substituted 3- to 10-membered heterocycloalkyl.

Also provided are embodiments wherein any embodiment above may be combined with any one or more of these embodiments, provided the combination is not mutually exclusive.

As used herein, two embodiments are “mutually exclusive” when one is defined to be something which is different than the other. For example, an embodiment wherein two groups combine to form a cycloalkyl is mutually exclusive with an embodiment in which one group is ethyl the other group is hydrogen. Similarly, an embodiment wherein one group is CH₂ is mutually exclusive with an embodiment wherein the same group is NH.

That is, any combination of the groups described above for the various variables is contemplated herein. Throughout the specification, groups and substituents thereof are chosen by one skilled in the field to provide stable moieties and compounds.

Exemplary compounds of the invention include the compounds described in the following Tables:

TABLE 1

| Cmpd | | |
|---|---|---|
| # | Structure | Name |
| 1 | 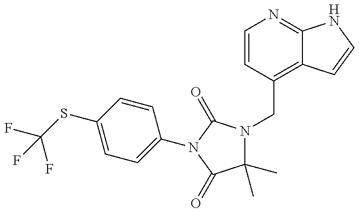 [SMILES: CC1(C)C(=O)N(c2ccc(SC(F)(F)F)cc2)C(=O)N1Cc1ccnc2[nH]ccc12] | 1-((1H-pyrrolo[2,3-b]pyridin-4-yl)methyl)- 5,5-dimethyl-3-(4- ((trifluoromethyl)thio)phenyl)imidazolidine- 2,4-dione |
| 2 | 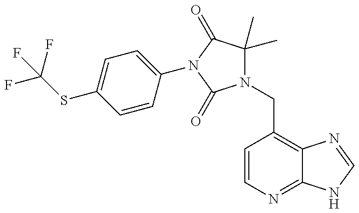 [SMILES: CC1(C)C(=O)N(c2ccc(SC(F)(F)F)cc2)C(=O)N1Cc1ccnc2[nH]cnc12] | 1-((3H-imidazo[4,5-b]pyridin-7-yl)methyl)- 5,5-dimethyl-3-(4- ((trifluoromethyl)thio)phenyl)imidazolidine- 2,4-dione |
| 3 | 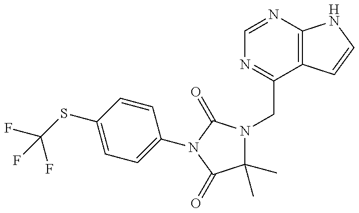 [SMILES: CC1(C)C(=O)N(c2ccc(SC(F)(F)F)cc2)C(=O)N1Cc1ncnc2[nH]ccc12] | 1-((7H-pyrrolo[2,3-d]pyrimidin-4- yl)methyl)-5,5-dimethyl-3-(4- ((trifluoromethyl)thio)phenyl)imidazolidine- 2,4-dione |
| 4 | 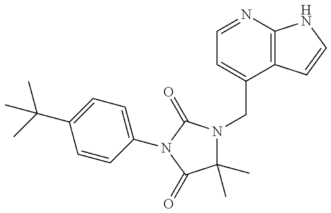 [SMILES: CC(C)(C)c1ccc(N2C(=O)N(Cc3ccnc4[nH]ccc34)C(C)(C)C2=O)cc1] | 1-((1H-pyrrolo[2,3-b]pyridin-4-yl)methyl)- 3-(4-tert-butyl)phenyl)-5,5- dimethylimidazolidine-2,4-dione |
| 5 | 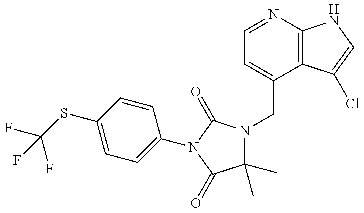 [SMILES: CC1(C)C(=O)N(c2ccc(SC(F)(F)F)cc2)C(=O)N1Cc1ccnc2[nH]cc(Cl)c12] | 1-((3-chloro-1H-pyrrolo[2,3-b]pyridin-4- yl)methyl)-5,5-dimethyl-3-(4- ((trifluoromethyl)thio)phenyl)imidazolidine- 2,4-dione |
| 6 | 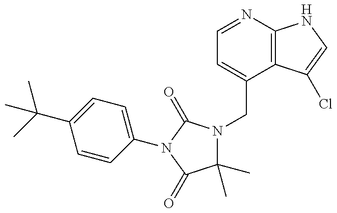 [SMILES: CC(C)(C)c1ccc(N2C(=O)N(Cc3ccnc4[nH]cc(Cl)c34)C(C)(C)C2=O)cc1] | 3-(4-(tert-butyl)phenyl)-1-((3-chloro-1H- pyrrolo[2,3-b]pyridin-4-yl)methyl)-5,5- dimethylimidazolidine-2,4-dione |
| 7 | 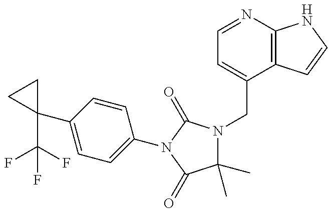 [SMILES: CC1(C)C(=O)N(c2ccc(C3(C(F)(F)F)CC3)cc2)C(=O)N1Cc1ccnc2[nH]ccc12] | 1-((1H-pyrrolo[2,3-b]pyridin-4-yl)methyl)- 5,5-dimethyl-3-(4-(1- (trifluoromethyl)cyclopropyl)phenyl) imidazolidine-2,4-dione |
| 8 | 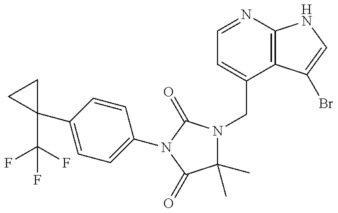 [SMILES: CC1(C)C(=O)N(c2ccc(C3(C(F)(F)F)CC3)cc2)C(=O)N1Cc1ccnc2[nH]cc(Br)c12] | 1-((3-bromo-1H-pyrrolo[2,3-b]pyridin-4- yl)methyl)-5,5-dimethyl-3-(4-(1- (trifluoromethyl)cyclopropyl)phenyl) imidazolidine-2,4-dione |
| 9 | 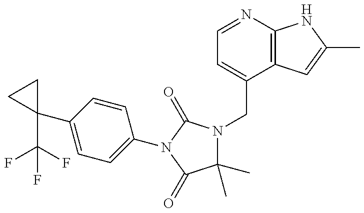 [SMILES: Cc1cc2c(CN3C(=O)N(c4ccc(C5(C(F)(F)F)CC5)cc4)C(=O)C3(C)C)ccnc2[nH]1] | 5,5-dimethyl-1-((2-methyl-1H-pyrrolo[2,3- b]pyridin-4-yl)methyl)-3-(4-(1- (trifluoromethyl)cyclopropyl)phenyl) imidazolidine-2,4-dione |
| 10 | 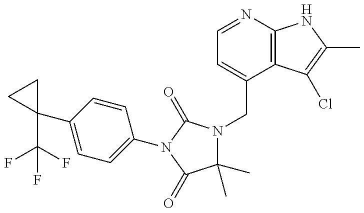 [SMILES: Cc1[nH]c2nccc(CN3C(=O)N(c4ccc(C5(C(F)(F)F)CC5)cc4)C(=O)C3(C)C)c2c1Cl] | 1-((3-chloro-2-methyl-1H-pyrrolo[2,3- b]pyridin-4-yl)methyl)-5,5-dimethyl-3-(4- (1-(trifluoromethyl)cyclopropyl)phenyl) imidazolidine-2,4-dione |
| 11 | 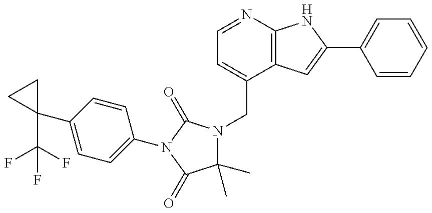 [SMILES: CC1(C)C(=O)N(c2ccc(C3(C(F)(F)F)CC3)cc2)C(=O)N1Cc1ccnc2[nH]c(-c3ccccc3)cc12] | 5,5-dimethyl-1-((2-phenyl-1H-pyrrolo[2,3- b]pyridin-4-yl)methyl)-3-(4-(1- (trifluoromethyl)cyclopropyl)phenyl) imidazolidine-2,4-dione |
| 12 | 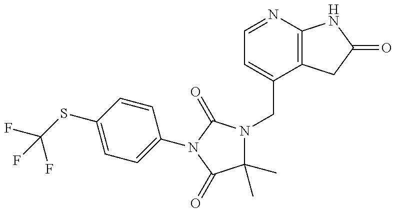 [SMILES: CC1(C)C(=O)N(c2ccc(SC(F)(F)F)cc2)C(=O)N1Cc1ccnc2c1CC(=O)N2] | 5,5-dimethyl-1-((2-oxo-2,3-dihydro-1H- pyrrolo[2,3-b]pyridin-4-yl)methyl)-3-(4- ((trifluoromethyl)thio)phenyl)imidazolidine- 2,4-dione |
| 13 | 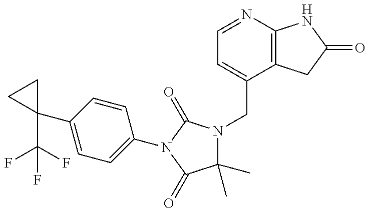 [SMILES: CC1(C)C(=O)N(c2ccc(C3(C(F)(F)F)CC3)cc2)C(=O)N1Cc1ccnc2c1CC(=O)N2] | 5,5-dimethyl-1-((2-oxo-2,3-dihydro-1H- pyrrolo[2,3-b]pyridin-4-yl)methyl)-3-(4-(1- (trifluoromethyl)cyclopropyl)phenyl) imidazolidine-2,4-dione |
| 14 | 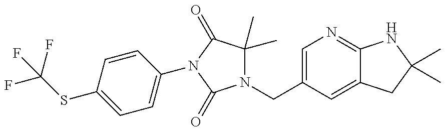 [SMILES: CC1(C)Cc2cc(CN3C(=O)N(c4ccc(SC(F)(F)F)cc4)C(=O)C3(C)C)cnc2N1] | 1-((2,2-dimethyl-2,3-dihydro-1H- pyrrolo[2,3-b]pyridin-5-yl)methyl)-5,5- dimethyl-3-(4- ((trifluoromethyl)thio)phenyl)imidazolidine- 2,4-dione |
| 15 | 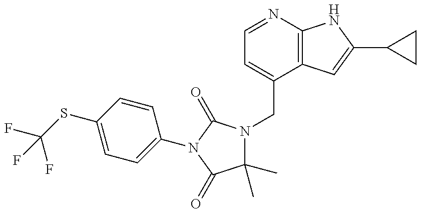 [SMILES: CC1(C)C(=O)N(c2ccc(SC(F)(F)F)cc2)C(=O)N1Cc1ccnc2[nH]c(C3CC3)cc12] | 1-((2-cyclopropyl-1H-pyrrolo[2,3-b]pyridin- 4-yl)methyl)-5,5-dimethyl-3-(4- ((trifluoromethyl)thio)phenyl)imidazolidine- 2,4-dione |
| 16 | 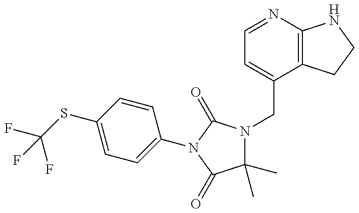 [SMILES: CC1(C)C(=O)N(c2ccc(SC(F)(F)F)cc2)C(=O)N1Cc1ccnc2c1CCN2] | 1-((2,3-dihydro-1H-pyrrolo[2,3-b]pyridin-4- yl)methyl)-5,5-dimethyl-3-(4- ((trifluoromethyl)thio)phenyl)imidazolidine- 2,4-dione |
| 17 | 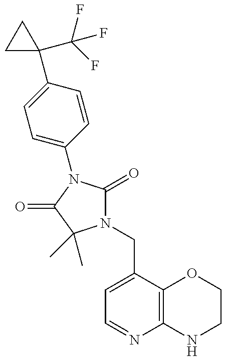 [SMILES: CC1(C)C(=O)N(c2ccc(C3(C(F)(F)F)CC3)cc2)C(=O)N1Cc1ccnc2c1OCCN2] | 1-((3,4-dihydro-2H-pyrido[3,2- b][1,4]oxazin-8-yl)methyl)-5,5-dimethyl-3- (4-(1- (trifluoromethyl)cyclopropyl)phenyl) imidazolidine-2,4-dione |
| 18 | 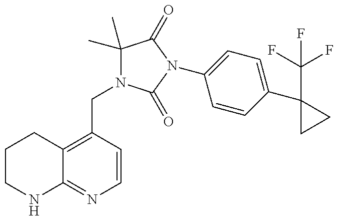 [SMILES: CC1(C)C(=O)N(c2ccc(C3(C(F)(F)F)CC3)cc2)C(=O)N1Cc1ccnc2c1CCCN2] | 5,5-dimethyl-1-((5,6,7,8-tetrahydro-1,8- naphthyridin-4-yl)methyl)-3-(4-(1- (trifluoromethyl)cyclopropyl)phenyl) imidazolidine-2,4-dione |
| 19 | 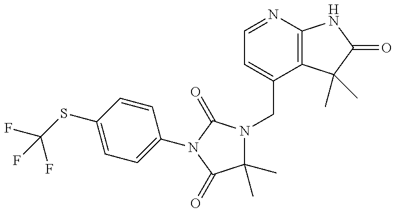 [SMILES: CC1(C)C(=O)Nc2nccc(CN3C(=O)N(c4ccc(SC(F)(F)F)cc4)C(=O)C3(C)C)c21] | 1-((3,3-dimethyl-2-oxo-2,3-dihydro-1H- pyrrolo[2,3-b]pyridin-4-yl)methyl)-5,5- dimethyl-3-(4- ((trifluoromethyl)thio)phenyl)imidazolidine- 2,4-dione |
| 20 | 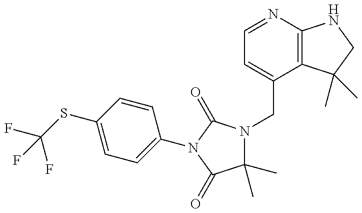 [SMILES: CC1(C)CNc2nccc(CN3C(=O)N(c4ccc(SC(F)(F)F)cc4)C(=O)C3(C)C)c21] | 1-((3,3-dimethyl-2,3-dihydro-1H- pyrrolo[2,3-b]pyridin-4-yl)methyl)-5,5- dimethyl-3-(4- ((trifluoromethyl)thio)phenyl)imidazolidine- 2,4-dione |
| 21 | 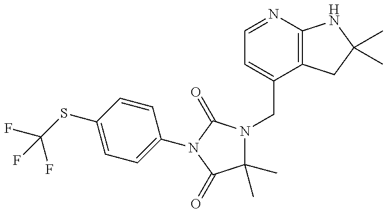 [SMILES: CC1(C)Cc2c(CN3C(=O)N(c4ccc(SC(F)(F)F)cc4)C(=O)C3(C)C)ccnc2N1] | 1-((2,2-dimethyl-2,3-dihydro-1H- pyrrolo[2,3-b]pyridin-4-yl)methyl)-5,5- dimethyl-3-(4- ((trifluoromethyl)thio)phenyl)imidazolidine- 2,4-dione |
| 22 | 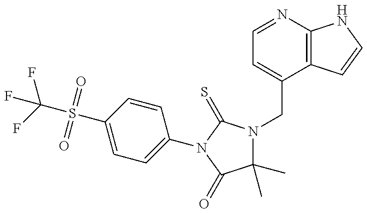 [SMILES: CC1(C)C(=O)N(c2ccc(S(=O)(=O)C(F)(F)F)cc2)C(=S)N1Cc1ccnc2[nH]ccc12] | 1-((1H-pyrrolo[2,3-b]pyridin-4-yl)methyl)- 5,5-dimethyl-3-(4- ((trifluoromethyl)sulfonyl)phenyl) imidazolidine-2,4-dione |
| 23 | 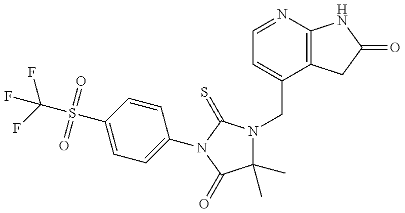 [SMILES: CC1(C)C(=O)N(c2ccc(S(=O)(=O)C(F)(F)F)cc2)C(=S)N1Cc1ccnc2c1CC(=O)N2] | 5,5-dimethyl-1-((2-oxo-2,3-dihydro-1H- pyrrolo[2,3-b]pyridin-4-yl)methyl)-3-(4- ((trifluoromethyl)sulfonyl)phenyl) imidazolidine-2,4-dione |
| 24 | 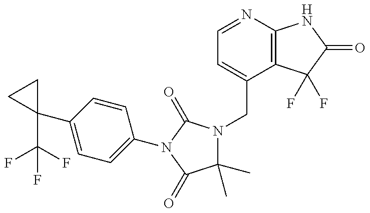 [SMILES: CC1(C)C(=O)N(c2ccc(C3(C(F)(F)F)CC3)cc2)C(=O)N1Cc1ccnc2c1C(F)(F)C(=O)N2] | 1-((3,3-difluoro-2-oxo-2,3-dihydro-1H- pyrrolo[2,3-b]pyridin-4-yl)methyl)-5,5- dimethyl-3-(4-(1- (trifluoromethyl)cyclopropyl)phenyl) imidazolidine-2,4-dione |
| 25 | 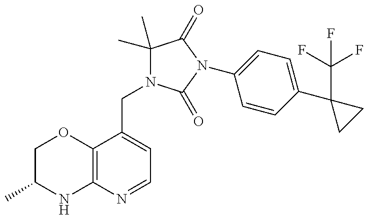 [SMILES: C[C@@H]1COc2c(CN3C(=O)N(c4ccc(C5(C(F)(F)F)CC5)cc4)C(=O)C3(C)C)ccnc2N1] | (R)-5,5-dimethyl-1-((3-methyl-3,4-dihydro- 2H-pyrido[3,2-b][1,4]oxazin-8-yl)methyl)-3-(4-(1- (trifluoromethyl)cyclopropyl)phenyl) imidazolidine-2,4-dione |
| 26 | 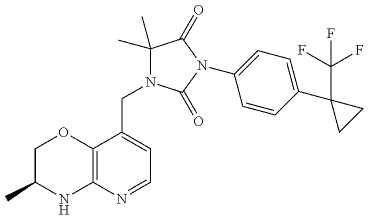 [SMILES: C[C@H]1COc2c(CN3C(=O)N(c4ccc(C5(C(F)(F)F)CC5)cc4)C(=O)C3(C)C)ccnc2N1] | (S)-5,5-dimethyl-1-((3-methyl-3,4-dihydro- 2H-pyrido[3,2-b][1,4]oxazin-8-yl)methyl)- 3-(4-(1- (trifluoromethyl)cyclopropyl)phenyl) imidazolidine-2,4-dione |
| 27 | 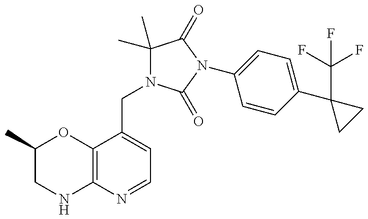 [SMILES: C[C@@H]1CNc2nccc(CN3C(=O)N(c4ccc(C5(C(F)(F)F)CC5)cc4)C(=O)C3(C)C)c2O1] | (R)-5,5-dimethyl-1-((2-methyl-3,4-dihydro- 2H-pyrido[3,2-b][1,4]oxazin-8-yl)methyl)- 3-(4-(1- (trifluoromethyl)cyclopropyl)phenyl) imidazolidine-2,4-dione |
| 28 | 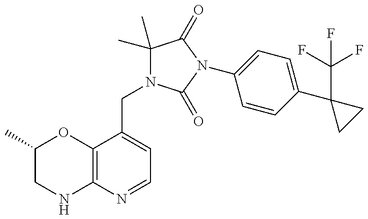 [SMILES: C[C@H]1CNc2nccc(CN3C(=O)N(c4ccc(C5(C(F)(F)F)CC5)cc4)C(=O)C3(C)C)c2O1] | (S)-5,5-dimethyl-1-((2-methyl-3,4-dihydro- 2H-pyrido[3,2-b][1,4]oxazin-8-yl)methyl)- 3-(4-(1- (trifluoromethyl)cyclopropyl)phenyl) imidazolidine-2,4-dione |
| 29 | 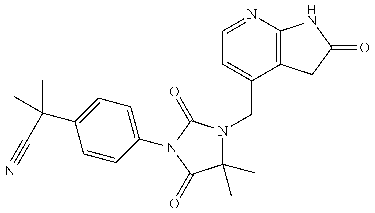 [SMILES: CC(C)(C#N)c1ccc(N2C(=O)N(Cc3ccnc4c3CC(=O)N4)C(C)(C)C2=O)cc1] | 2-(4-(4,4-dimethyl-2,5-dioxo-3-((2-oxo-2,3- dihydro-1H-pyrrolo[2,3-b]pyridin-4- yl)methyl)imidazolidin-1-yl)phenyl)-2- methylpropanenitrile |
| 30 | 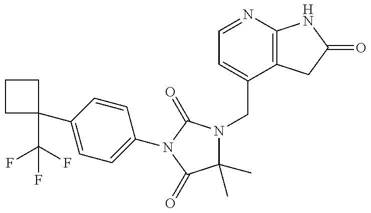 [SMILES: CC1(C)C(=O)N(c2ccc(C3(C(F)(F)F)CCC3)cc2)C(=O)N1Cc1ccnc2c1CC(=O)N2] | 5,5-dimethyl-1-((2-oxo-2,3-dihydro-1H- pyrrolo[2,3-b]pyridin-4-yl)methyl)-3-(4-(1- (trifluoromethyl)cyclobutyl)phenyl) imidazolidine-2,4-dione |
| 31 | 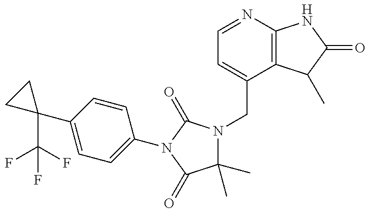 [SMILES: CC1C(=O)Nc2nccc(CN3C(=O)N(c4ccc(C5(C(F)(F)F)CC5)cc4)C(=O)C3(C)C)c21] | 5,5-dimethyl-1-((3-methyl-2-oxo-2,3- dihydro-1H-pyrrolo[2,3-b]pyridin-4- yl)methyl)-3-(4-(1- (trifluoromethyl)cyclopropyl)phenyl) imidazolidine-2,4-dione |
| 32 | 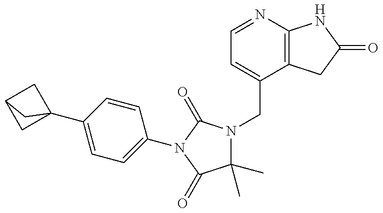 [SMILES: CC1(C)C(=O)N(c2ccc(C34CC(C3)C4)cc2)C(=O)N1Cc1ccnc2c1CC(=O)N2] | 3-(4-(bicyclo[1.1.1]pentan-1-yl)phenyl)-5,5- dimethyl-1-((2-oxo-2,3-dihydro-1H- pyrrolo[2,3-b]pyridin-4- yl)methyl)imidazolidine-2,4-dione |
| 33 | 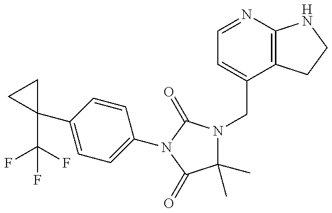 [SMILES: CC1(C)C(=O)N(c2ccc(C3(C(F)(F)F)CC3)cc2)C(=O)N1Cc1ccnc2c1CCN2] | 1-((2,3-dihydro-1H-pyrrolo[2,3-b]pyridin-4- yl)methyl)-5,5-dimethyl-3-(4-(1- (trifluoromethyl)cyclopropyl)phenyl) imidazolidine-2,4-dione |
| 34 | 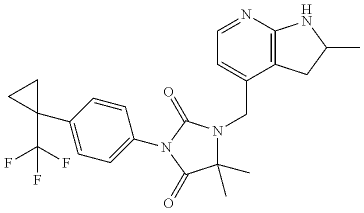 [SMILES: CC1Cc2c(CN3C(=O)N(c4ccc(C5(C(F)(F)F)CC5)cc4)C(=O)C3(C)C)ccnc2N1] | 5,5-dimethyl-1-((2-methyl-2,3-dihydro-1H- pyrrolo[2,3-b]pyridin-4-yl)methyl)-3-(4-(1- (trifluoromethyl)cyclopropyl)phenyl) imidazolidine-2,4-dione |
| 35 | 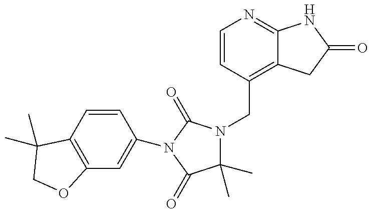 [SMILES: CC1(C)COc2cc(N3C(=O)N(Cc4ccnc5c4CC(=O)N5)C(C)(C)C3=O)ccc21] | 3-(3,3-dimethyl-2,3-dihydrobenzofuran-6- yl)-5,5-dimethyl-1-((2-oxo-2,3-dihydro-1H- pyrrolo[2,3-b]pyridin-4- yl)methyl)imidazolidine-2,4-dione |
| 36 | 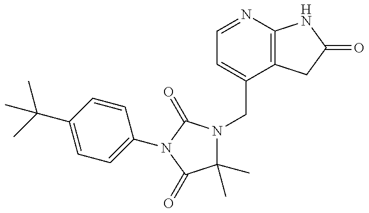 [SMILES: CC(C)(C)c1ccc(N2C(=O)N(Cc3ccnc4c3CC(=O)N4)C(C)(C)C2=O)cc1] | 3-(4-(tert-butyl)phenyl)-5,5-dimethyl-1-((2- oxo-2,3-dihydro-1H-pyrrolo[2,3-b]pyridin- 4-yl)methyl)imidazolidine-2,4-dione |
| 37 | 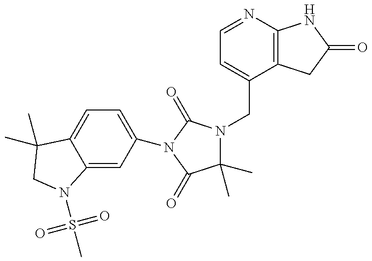 [SMILES: CC1(C)CN(S(C)(=O)=O)c2cc(N3C(=O)N(Cc4ccnc5c4CC(=O)N5)C(C)(C)C3=O)ccc21] | 3-(3,3-dimethyl-1-(methylsulfonyl)indolin- 6-yl)-5,5-dimethyl-1-((2-oxo-2,3-dihydro- 1H-pyrrolo[2,3-b]pyridin-4- yl)methyl)imidazolidine-2,4-dione |
| 38 | 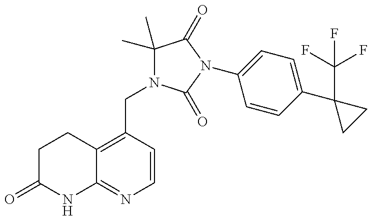 [SMILES: CC1(C)C(=O)N(c2ccc(C3(C(F)(F)F)CC3)cc2)C(=O)N1Cc1ccnc2c1CCC(=O)N2] | 5,5-dimethyl-1-((7-oxo-5,6,7,8-tetrahydro- 1,8-naphthyridin-4-yl)methyl)-3-(4-(1- (trifluoromethyl)cyclopropyl)phenyl) imidazolidine-2,4-dione |
| 39 | 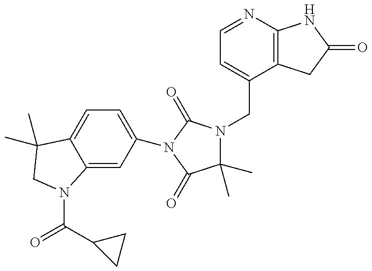 [SMILES: CC1(C)CN(C(=O)C2CC2)c2cc(N3C(=O)N(Cc4ccnc5c4CC(=O)N5)C(C)(C)C3=O)ccc21] | 3-(1-(cyclopropanecarbonyl)-3,3- dimethylindolin-6-yl)-5,5-dimethyl-1-((2- oxo-2,3-dihydro-1H-pyrrolo[2,3-b]pyridin- 4-yl)methyl)imidazolidine-2,4-dione |
| 40 | 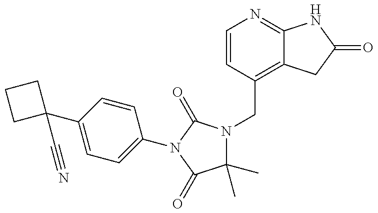 [SMILES: CC1(C)C(=O)N(c2ccc(C3(C#N)CCC3)cc2)C(=O)N1Cc1ccnc2c1CC(=O)N2] | 1-(4-(4,4-dimethyl-2,5-dioxo-3-((2-oxo-2,3- dihydro-1H-pyrrolo[2,3-b]pyridin-4- yl)methyl)imidazolidin-1- yl)phenyl)cyclobutane-1-carbonitrile |
| 41 | 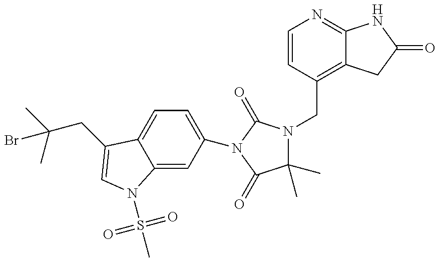 [SMILES: CC(C)(Br)Cc1cn(S(C)(=O)=O)c2cc(N3C(=O)N(Cc4ccnc5c4CC(=O)N5)C(C)(C)C3=O)ccc12] | 3-(3-(2-bromo-2-methylpropyl)-1- (methylsulfonyl)-1H-indol-6-yl)-5,5- dimethyl-1-((2-oxo-2,3-dihydro-1H- pyrrolo[2,3-b]pyridin-4- yl)methyl)imidazolidine-2,4-dione |
| 42 | 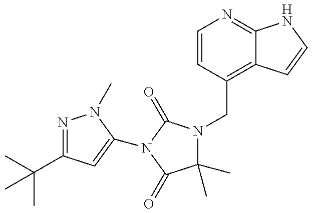 [SMILES: Cn1nc(C(C)(C)C)cc1N1C(=O)N(Cc2ccnc3[nH]ccc23)C(C)(C)C1=O] | 1-((1H-pyrrolo[2,3-b]pyridin-4-yl)methyl)- 3-(3-(tert-butyl)-1-methyl-1H-pyrazol-5-yl)- 5,5-dimethylimidazolidine-2,4-dione |
| 43 | 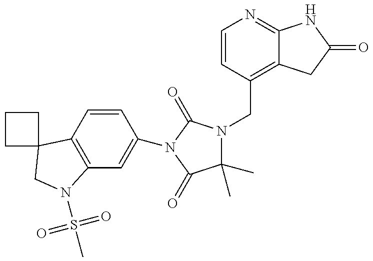 [SMILES: CC1(C)C(=O)N(c2ccc3c(c2)N(S(C)(=O)=O)CC32CCC2)C(=O)N1Cc1ccnc2c1CC(=O)N2] | 5,5-dimethyl-3-(1′- (methylsulfonyl)spiro[cyclobutane-1,3′- indolin]-6′-yl)-1-((2-oxo-2,3-dihydro-1H- pyrrolo[2,3-b]pyridin-4- yl)methyl)imidazolidine-2,4-dione |
| 44 | 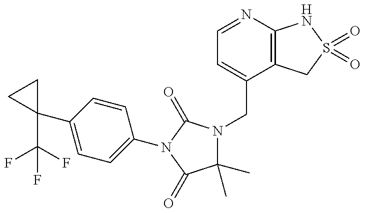 [SMILES: CC1(C)C(=O)N(c2ccc(C3(C(F)(F)F)CC3)cc2)C(=O)N1Cc1ccnc2c1CS(=O)(=O)N2] | 1-((2,2-dioxido-1,3-dihydroisothiazolo[3,4- b]pyridin-4-yl)methyl)-5,5-dimethyl-3-(4- (1-(trifluoromethyl)cyclopropyl)phenyl) imidazolidine-2,4-dione |
| 45 | 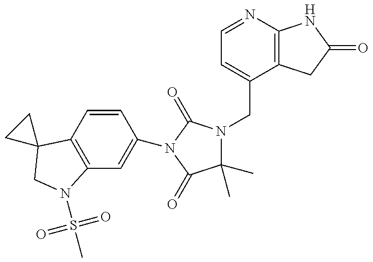 [SMILES: CC1(C)C(=O)N(c2ccc3c(c2)N(S(C)(=O)=O)CC32CC2)C(=O)N1Cc1ccnc2c1CC(=O)N2] | 5,5-dimethyl-3-(1′- (methylsulfonyl)spiro[cyclopropane-1,3′- indolin]-6′-yl)-1-((2-oxo-2,3-dihydro-1H- pyrrolo[2,3-b]pyridin-4- yl)methyl)imidazolidine-2,4-dione |
| 46 | 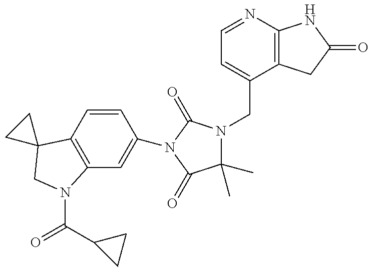 [SMILES: CC1(C)C(=O)N(c2ccc3c(c2)N(C(=O)C2CC2)CC32CC2)C(=O)N1Cc1ccnc2c1CC(=O)N2] | 3-(1′- (cyclopropanecarbonyl)spiro[cyclopropane- 1,3′-indolin]-6′-yl)-5,5-dimethyl-1-((2- oxo-2,3-dihydro-1H-pyrrolo[2,3-b]pyridin- 4-yl)methyl)imidazolidine-2,4-dione |
| 47 | 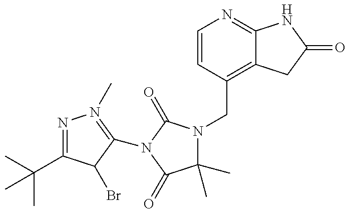 | 3-(4-bromo-3-(tert-butyl)-1-methyl-1H- pyrazol-5-yl)-5,5-dimethyl-1-((2-oxo-2,3- dihydro-1H-pyrrolo[2,3-b]pyridin-4- yl)methyl)imidazolidine-2,4-dione |
| 48 | 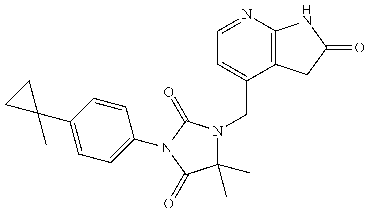 [SMILES: CC1(c2ccc(N3C(=O)N(Cc4ccnc5c4CC(=O)N5)C(C)(C)C3=O)cc2)CC1] | 5,5-dimethyl-3-(4-(1- methylcyclopropyl)phenyl)-1-((2-oxo-2,3- dihydro-1H-pyrrolo[2,3-b]pyridin-4- yl)methyl)imidazolidine-2,4-dione |
| 49 | 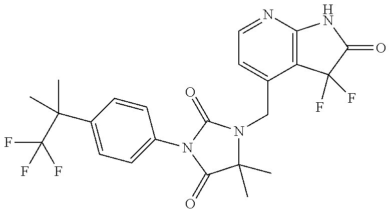 [SMILES: CC1(C)C(=O)N(c2ccc(C(C)(C)C(F)(F)F)cc2)C(=O)N1Cc1ccnc2c1C(F)(F)C(=O)N2] | 1-((3,3-difluoro-2-oxo-2,3-dihydro-1H- pyrrolo[2,3-b]pyridin-4-yl)methyl)-5,5- dimethyl-3-(4-(1,1,1-trifluoro-2- methylpropan-2-yl)phenyl)imidazolidine- 2,4-dione |
| 50 | 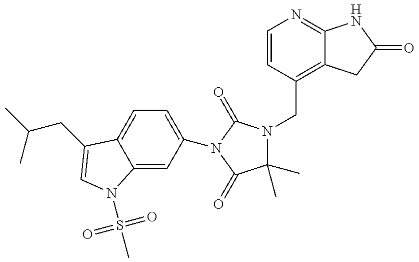 [SMILES: CC(C)Cc1cn(S(C)(=O)=O)c2cc(N3C(=O)N(Cc4ccnc5c4CC(=O)N5)C(C)(C)C3=O)ccc12] | 3-(3-isobutyl-1-(methylsulfonyl)-1H-indol- 6-yl)-5,5-dimethyl-1-((2-oxo-2,3-dihydro- 1H-pyrrolo[2,3-b]pyridin-4- yl)methyl)imidazolidine-2,4-dione |
| 51 | 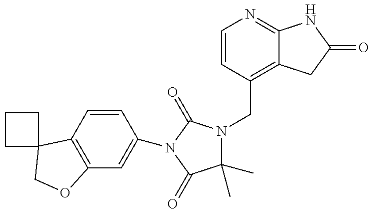 [SMILES: CC1(C)C(=O)N(c2ccc3c(c2)OCC32CCC2)C(=O)N1Cc1ccnc2c1CC(=O)N2] | 5,5-dimethyl-1-((2-oxo-2,3-dihydro-1H- pyrrolo[2,3-b]pyridin-4-yl)methyl)-3-(2H- spiro[benzofuran-3,1′-cyclobutan]-6- yl)imidazolidine-2,4-dione |
| 52 | 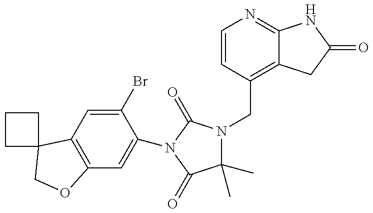 [SMILES: CC1(C)C(=O)N(c2cc3c(cc2Br)C2(CCC2)CO3)C(=O)N1Cc1ccnc2c1CC(=O)N2] | 3-(5-bromo-2H-spiro[benzofuran-3,1′- cyclobutan]-6-yl)-5,5-dimethyl-1-((2-oxo- 2,3-dihydro-1H-pyrrolo[2,3-b]pyridin-4- yl)methyl)imidazolidine-2,4-dione |
| 53 | 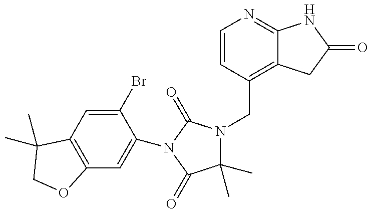 [SMILES: CC1(C)COc2cc(N3C(=O)N(Cc4ccnc5c4CC(=O)N5)C(C)(C)C3=O)c(Br)cc21] | 3-(5-bromo-3,3-dimethyl-2,3- dihydrobenzofuran-6-yl)-5,5-dimethyl-1- ((2-oxo-2,3-dihydro-1H-pyrrolo[2,3-b] pyridin-4-yl)methyl)imidazolidine-2,4- dione |
| 55 | 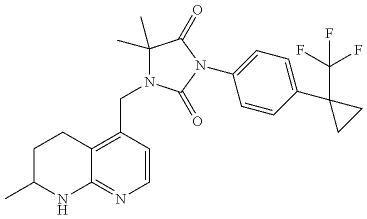 [SMILES: CC1CCc2c(CN3C(=O)N(c4ccc(C5(C(F)(F)F)CC5)cc4)C(=O)C3(C)C)ccnc2N1] | 5,5-dimethyl-1-((7-methyl-5,6,7,8- tetrahydro-1,8-naphthyridin-4-yl)methyl)-3- (4-(1- (trifluoromethyl)cyclopropyl)phenyl) imidazolidine-2,4-dione |
| 56 | 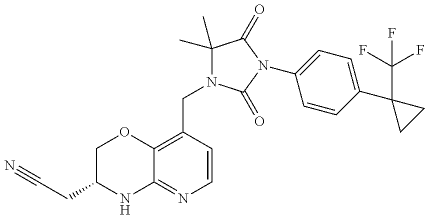 [SMILES: CC1(C)C(=O)N(c2ccc(C3(C(F)(F)F)CC3)cc2)C(=O)N1Cc1ccnc2c1OC[C@@H](CC#N)N2] | (R)-2-(8-((5,5-dimethyl-2,4-dioxo-3-(4-(1- (trifluoromethyl)cyclopropyl)phenyl) imidazolidin-1-yl)methyl)-3,4-dihydro- 2H-pyrido[3,2-b][1,4]oxazin-3-yl) acetonitrile |
| 57 | 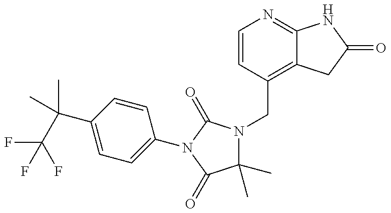 [SMILES: CC1(C)C(=O)N(c2ccc(C(C)(C)C(F)(F)F)cc2)C(=O)N1Cc1ccnc2c1CC(=O)N2] | 5,5-dimethyl-1-((2-oxo-2,3-dihydro-1H- pyrrolo[2,3-b]pyridin-4-yl)methyl)-3-(4- (1,1,1-trifluoro-2-methylpropan-2- yl)phenyl)imidazolidine-2,4-dione |
| 58 | 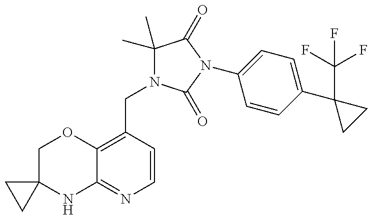 [SMILES: CC1(C)C(=O)N(c2ccc(C3(C(F)(F)F)CC3)cc2)C(=O)N1Cc1ccnc2c1OCC1(CC1)N2] | 1-((2′H,4′H-spiro[cyclopropane-1,3′- pyrido[3,2-b][1,4]oxazin]-8′-yl)methyl)- 5,5-dimethyl-3-(4-(1- (trifluoromethyl)cyclopropyl)phenyl) imidazolidine-2,4-dione |
| 59 | 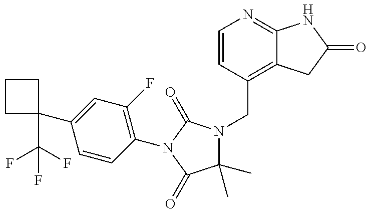 [SMILES: CC1(C)C(=O)N(c2ccc(C3(C(F)(F)F)CCC3)cc2F)C(=O)N1Cc1ccnc2c1CC(=O)N2] | 3-(2-fluoro-4-(1- (trifluoromethyl)cyclobutyl)phenyl)-5,5- dimethyl-1-((2-oxo-2,3-dihydro-1H- pyrrolo[2,3-b]pyridin-4- yl)methyl)imidazolidine-2,4-dione |
| 60 | 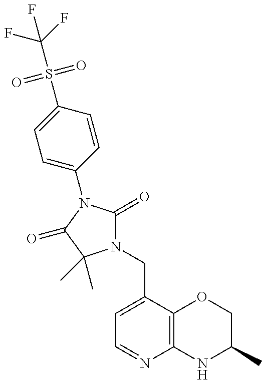 [SMILES: C[C@@H]1COc2c(CN3C(=O)N(c4ccc(S(=O)(=O)C(F)(F)F)cc4)C(=O)C3(C)C)ccnc2N1] | (R)-5,5-dimethyl-1-((3-methyl-3,4-dihydro- 2H-pyrido[3,2-b][1,4]oxazin-8-yl)methyl)- 3-(4-((trifluoromethyl)sulfonyl)phenyl) imidazolidine-2,4-dione |
| 61 | 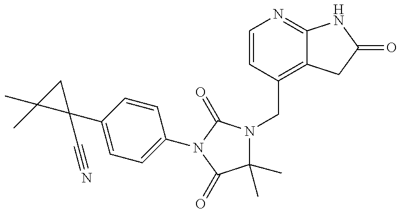 [SMILES: CC1(C)C(=O)N(c2ccc(C3(C#N)CC3(C)C)cc2)C(=O)N1Cc1ccnc2c1CC(=O)N2] | 1-(4-(4,4-dimethyl-2,5-dioxo-3-((2-oxo-2,3- dihydro-1H-pyrrolo[2,3-b]pyridin-4- yl)methyl)imidazolidin-1-yl)phenyl)-2,2- dimethylcyclopropane-1-carbonitrile |
| 62 | 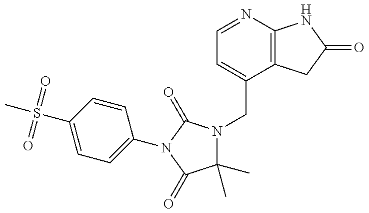 [SMILES: CC1(C)C(=O)N(c2ccc(S(C)(=O)=O)cc2)C(=O)N1Cc1ccnc2c1CC(=O)N2] | 5,5-dimethyl-3-(4-(methylsulfonyl)phenyl)- 1-((2-oxo-2,3-dihydro-1H-pyrrolo[2,3- b]pyridin-4-yl)methyl)imidazolidine-2,4- dione |
| 63 | 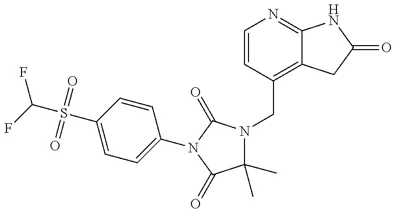 [SMILES: CC1(C)C(=O)N(c2ccc(S(=O)(=O)C(F)F)cc2)C(=O)N1Cc1ccnc2c1CC(=O)N2] | 3-(4-((difluoromethyl)sulfonyl)phenyl)-5,5- dimethyl-1-((2-oxo-2,3-dihydro-1H- pyrrolo[2,3-b]pyridin-4- yl)methyl)imidazolidine-2,4-dione |
| 64 | 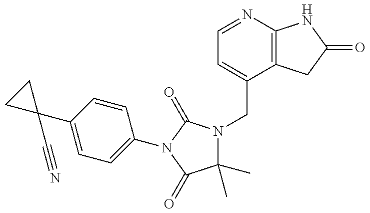 [SMILES: CC1(C)C(=O)N(c2ccc(C3(C#N)CC3)cc2)C(=O)N1Cc1ccnc2c1CC(=O)N2] | 1-(4-(4,4-dimethyl-2,5-dioxo-3-((2-oxo-2,3- dihydro-1H-pyrrolo[2,3-b]pyridin-4- yl)methyl)imidazolidin-1- yl)phenyl)cyclopropane-1-carbonitrile |
| 65 | 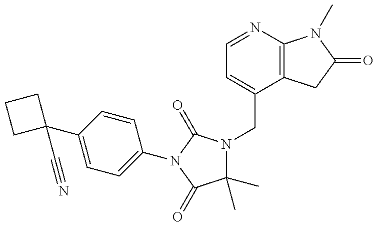 [SMILES: CN1C(=O)Cc2c(CN3C(=O)N(c4ccc(C5(C#N)CCC5)cc4)C(=O)C3(C)C)ccnc21] | 1-(4-(4,4-dimethyl-3-((1-methyl-2-oxo-2,3- dihydro-1H-pyrrolo[2,3-b]pyridin-4- yl)methyl)-2,5-dioxoimidazolidin-1- yl)phenyl)cyclobutane-1-carbonitrile |
| 66 | 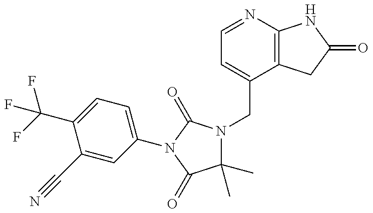 [SMILES: CC1(C)C(=O)N(c2ccc(C(F)(F)F)c(C#N)c2)C(=O)N1Cc1ccnc2c1CC(=O)N2] | 5-(4,4-dimethyl-2,5-dioxo-3-((2-oxo-2,3- dihydro-1H-pyrrolo[2,3-b]pyridin-4- yl)methyl)imidazolidin-1-yl)-2- (trifluoromethyl)benzonitrile |
| 67 | 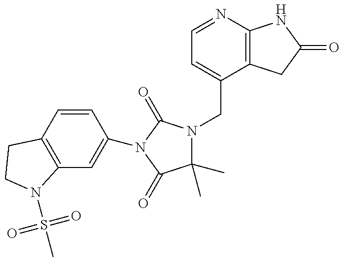 [SMILES: CC1(C)C(=O)N(c2ccc3c(c2)N(S(C)(=O)=O)CC3)C(=O)N1Cc1ccnc2c1CC(=O)N2] | 5,5-dimethyl-3-(1-(methylsulfonyl)indolin- 6-yl)-1-((2-oxo-2,3-dihydro-1H-pyrrolo [2,3-b]pyridin-4-yl)methyl)imidazolidine- 2,4-dione |
| 68 | 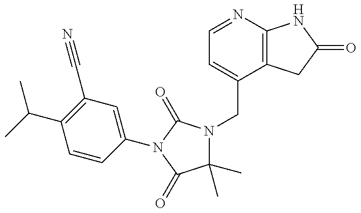 [SMILES: CC(C)c1ccc(N2C(=O)N(Cc3ccnc4c3CC(=O)N4)C(C)(C)C2=O)cc1C#N] | 5-(4,4-dimethyl-2,5-dioxo-3-((2-oxo-2,3- dihydro-1H-pyrrolo[2,3-b]pyridin-4- yl)methyl)imidazolidin-1-yl)-2- isopropylbenzonitrile |
| 69 | 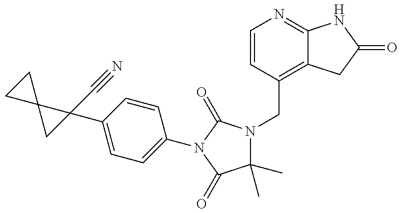 [SMILES: CC1(C)C(=O)N(c2ccc(C3(C#N)CC34CC4)cc2)C(=O)N1Cc1ccnc2c1CC(=O)N2] | 1-(4-(4,4-dimethyl-2,5-dioxo-3-((2-oxo-2,3- dihydro-1H-pyrrolo[2,3-b]pyridin-4- yl)methyl)imidazolidin-1- yl)phenyl)spiro[2.2]pentane-1- carbonitrile |
| 70 | 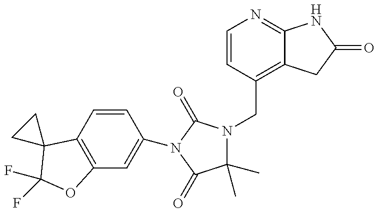 [SMILES: CC1(C)C(=O)N(c2ccc3c(c2)OC(F)(F)C32CC2)C(=O)N1Cc1ccnc2c1CC(=O)N2] | 3-(2,2-difluoro-2H-spiro[benzofuran-3,1′- cyclopropan]-6-yl)-5,5-dimethyl-1-((2- oxo-2,3-dihydro-1H-pyrrolo[2,3-b]pyridin- 4-yl)methyl)imidazolidine-2,4-dione |
| 71 | 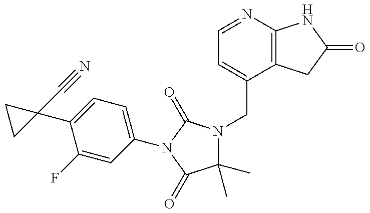 [SMILES: CC1(C)C(=O)N(c2ccc(C3(C#N)CC3)c(F)c2)C(=O)N1Cc1ccnc2c1CC(=O)N2] | 1-(4-(4,4-dimethyl-2,5-dioxo-3-((2-oxo-2,3- dihydro-1H-pyrrolo[2,3-b]pyridin-4- yl)methyl)imidazolidin-1-yl)-2- fluorophenyl)cyclopropane-1-carbonitrile |
| 72 | 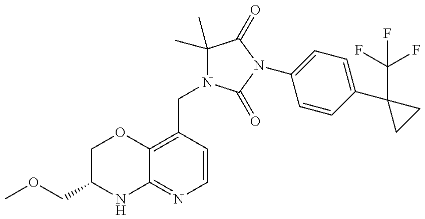 [SMILES: COC[C@@H]1COc2c(CN3C(=O)N(c4ccc(C5(C(F)(F)F)CC5)cc4)C(=O)C3(C)C)ccnc2N1] | (R)-1-((3-(methoxymethyl)-3,4-dihydro-2H- pyrido[3,2-b][1,4]oxazin-8-yl)methyl)-5,5- dimethyl-3-(4-(1- (trifluoromethyl)cyclopropyl)phenyl) imidazolidine-2,4-dione |
| 76 | 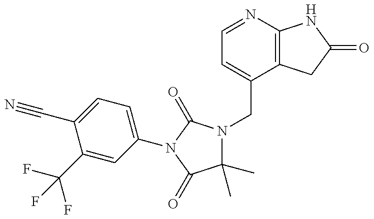 [SMILES: CC1(C)C(=O)N(c2ccc(C#N)c(C(F)(F)F)c2)C(=O)N1Cc1ccnc2c1CC(=O)N2] | 4-(4,4-dimethyl-2,5-dioxo-3-((2-oxo-2,3- dihydro-1H-pyrrolo[2,3-b]pyridin-4- yl)methyl)imidazolidin-1-yl)-2- (trifluoromethyl)benzonitrile |
| 77 | 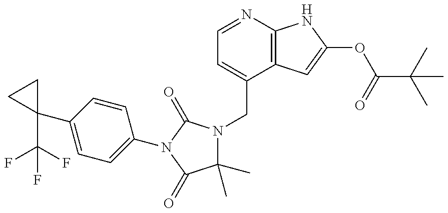 [SMILES: CC(C)(C)C(=O)Oc1cc2c(CN3C(=O)N(c4ccc(C5(C(F)(F)F)CC5)cc4)C(=O)C3(C)C)ccnc2[nH]1] | 4-((5,5-dimethyl-2,4-dioxo-3-(4-(1- (trifluoromethyl)cyclopropyl)phenyl) imidazolidin-1-yl)methyl)-1H-pyrrolo [2,3-b]pyridin-2-yl pivalate |
| 78 | 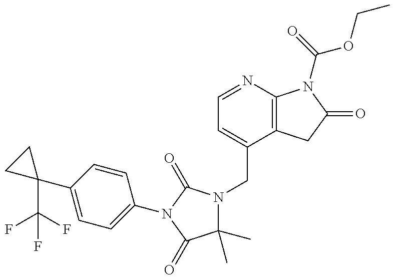 [SMILES: CCOC(=O)N1C(=O)Cc2c(CN3C(=O)N(c4ccc(C5(C(F)(F)F)CC5)cc4)C(=O)C3(C)C)ccnc21] | ethyl 4-((5,5-dimethyl-2,4-dioxo-3-(4-(1- (trifluoromethyl)cyclopropyl)phenyl) imidazolidin-1-yl)methyl)-2-oxo-2,3- dihydro-1H-pyrrolo[2,3-b]pyridine-1- carboxylate |
| 79 | 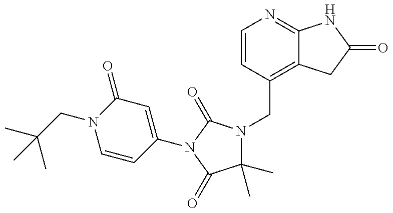 [SMILES: CC(C)(C)Cn1ccc(N2C(=O)N(Cc3ccnc4c3CC(=O)N4)C(C)(C)C2=O)cc1=O] | 5,5-dimethyl-3-(1-neopentyl-2-oxo-1,2- dihydropyridin-4-yl)-1-((2-oxo-2,3- dihydro-1H-pyrrolo[2,3-b]pyridin-4- yl)methyl)imidazolidine-2,4-dione |

In some embodiments, the compound is a pharmaceutically acceptable salt of a compound described in Table 1.

TABLE 2

| Cmpd # | Structure | Name |
|---|---|---|
| 80 | 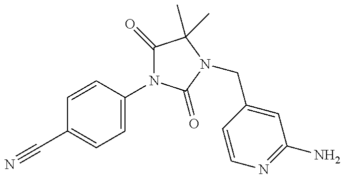 [SMILES: CC1(C)C(=O)N(c2ccc(C#N)cc2)C(=O)N1Cc1ccnc(N)c1] | 4-(3-((2-aminopyridin-4-yl)methyl)-4,4- dimethyl-2,5-dioxoimidazolidin-1- yl)benzonitrile |
| 81 | 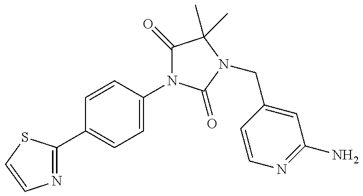 [SMILES: CC1(C)C(=O)N(c2ccc(-c3nccs3)cc2)C(=O)N1Cc1ccnc(N)c1] | 1-((2-aminopyridin-4-yl)methyl)-5,5- dimethyl-3-(4-(thiazol-2- yl)phenyl)imidazolidine-2,4-dione |
| 82 | 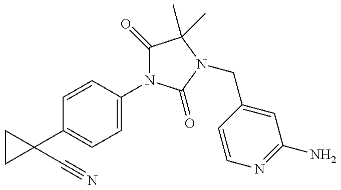 [SMILES: CC1(C)C(=O)N(c2ccc(C3(C#N)CC3)cc2)C(=O)N1Cc1ccnc(N)c1] | 1-(4-(3-((2-aminopyridin-4-yl)methyl)-4,4- dimethyl-2,5-dioxoimidazolidin-1- yl)phenyl)cyclopropane-1-carbonitrile |
| 83 | 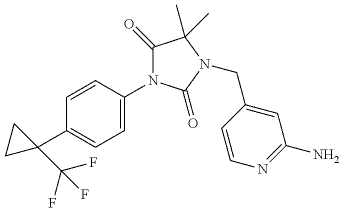 [SMILES: CC1(C)C(=O)N(c2ccc(C3(C(F)(F)F)CC3)cc2)C(=O)N1Cc1ccnc(N)c1] | 1-((2-aminopyridin-4-yl)methyl)-5,5- dimethyl-3-(4-(1- (trifluoromethyl)cyclopropyl)phenyl) imidazolidine-2,4-dione |
| 84 | 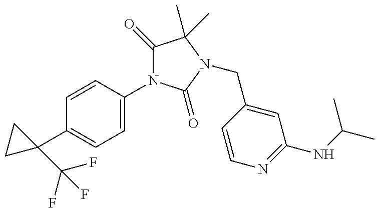 [SMILES: CC(C)Nc1cc(CN2C(=O)N(c3ccc(C4(C(F)(F)F)CC4)cc3)C(=O)C2(C)C)ccn1] | 1-((2-(isopropylamino)pyridin-4-yl)methyl)- 5,5-dimethyl-3-(4-(1- (trifluoromethyl)cyclopropyl)phenyl) imidazolidine-2,4-dione |
| 85 | 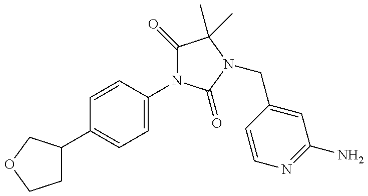 [SMILES: CC1(C)C(=O)N(c2ccc(C3CCOC3)cc2)C(=O)N1Cc1ccnc(N)c1] | 1-((2-aminopyridin-4-yl)methyl)-5,5- dimethyl-3-(4-(tetrahydrofuran-3- yl)phenyl)imidazolidine-2,4-dione |
| 86 | 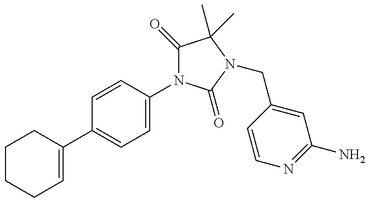 [SMILES: CC1(C)C(=O)N(c2ccc(C3=CCCCC3)cc2)C(=O)N1Cc1ccnc(N)c1] | 1-((2-aminopyridin-4-yl)methyl)-5,5- dimethyl-3-(2′,3′,4′,5′-tetrahydro-[1,1′- biphenyl]-4-yl)imidazolidine-2,4-dione |
| 87 | 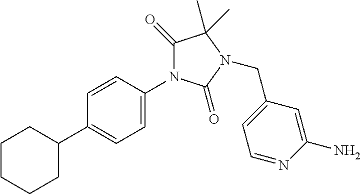 [SMILES: CC1(C)C(=O)N(c2ccc(C3CCCCC3)cc2)C(=O)N1Cc1ccnc(N)c1] | 1-((2-aminopyridin-4-yl)methyl)-3-(4- cyclohexylphenyl)-5,5- dimethylimidazolidine-2,4-dione |
| 88 | 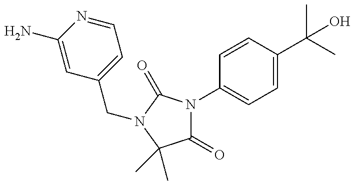 [SMILES: CC(C)(O)c1ccc(N2C(=O)N(Cc3ccnc(N)c3)C(C)(C)C2=O)cc1] | 1-((2-aminopyridin-4-yl)methyl)-3-(4-(2- hydroxypropan-2-yl)phenyl)-5,5- dimethylimidazolidine-2,4-dione |
| 89 | 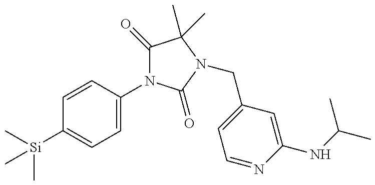 [SMILES: CC(C)Nc1cc(CN2C(=O)N(c3ccc([Si](C)(C)C)cc3)C(=O)C2(C)C)ccn1] | 1-((2-(isopropylamino)pyridin-4-yl)methyl)- 5,5-dimethyl-3-(4- (trimethylsilyl)phenyl)imidazolidine-2,4- dione |
| 90 | 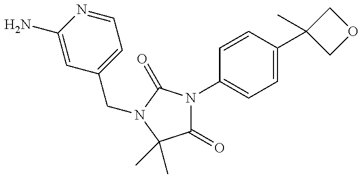 [SMILES: CC1(c2ccc(N3C(=O)N(Cc4ccnc(N)c4)C(C)(C)C3=O)cc2)COC1] | 1-((2-aminopyridin-4-yl)methyl)-5,5- dimethyl-3-(4-(3-methyloxetan-3- yl)phenyl)imidazolidine-2,4-dione |
| 91 | 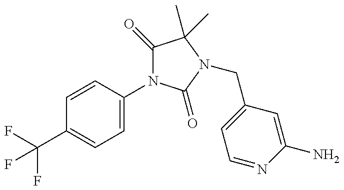 [SMILES: CC1(C)C(=O)N(c2ccc(C(F)(F)F)cc2)C(=O)N1Cc1ccnc(N)c1] | 1-((2-aminopyridin-4-yl)methyl)-5,5- dimethyl-3-(4- (trifluoromethyl)phenyl)imidazolidine-2,4- dione |
| 92 | 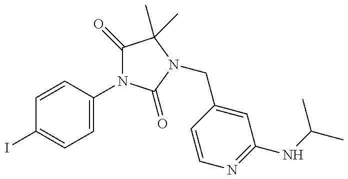 [SMILES: CC(C)Nc1cc(CN2C(=O)N(c3ccc(I)cc3)C(=O)C2(C)C)ccn1] | 3-(4-iodophenyl)-1-((2- (isopropylamino)pyridin-4-yl)methyl)-5,5- dimethylimidazolidine-2,4-dione |
| 93 | 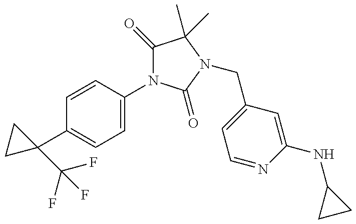 [SMILES: CC1(C)C(=O)N(c2ccc(C3(C(F)(F)F)CC3)cc2)C(=O)N1Cc1ccnc(NC2CC2)c1] | 1-((2-(cyclopropylamino)pyridin-4- yl)methyl)-5,5-dimethyl-3-(4-(1- (trifluoromethyl)cyclopropyl)phenyl) imidazolidine-2,4-dione |
| 94 | 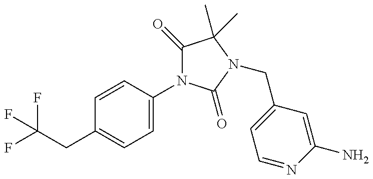 [SMILES: CC1(C)C(=O)N(c2ccc(CC(F)(F)F)cc2)C(=O)N1Cc1ccnc(N)c1] | 1-((2-aminopyridin-4-yl)methyl)-5,5- dimethyl-3-(4-(2,2,2- trifluoroethyl)phenyl)imidazolidine-2,4- dione |
| 95 | 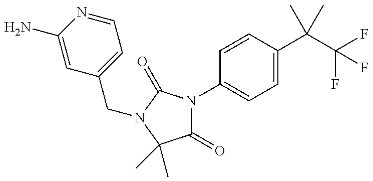 [SMILES: CC1(C)C(=O)N(c2ccc(C(C)(C)C(F)(F)F)cc2)C(=O)N1Cc1ccnc(N)c1] | 1-((2-aminopyridin-4-yl)methyl)-5,5- dimethyl-3-(4-(1,1,1-trifluoro-2- methylpropan-2-yl)phenyl)imidazolidine- 2,4-dione |
| 96 | 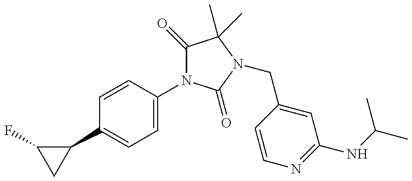 [SMILES: CC(C)Nc1cc(CN2C(=O)N(c3ccc([C@H]4C[C@@H]4F)cc3)C(=O)C2(C)C)ccn1] | 3-(4-((1R,2S)-2-fluorocyclopropyl)phenyl)- 1-((2-(isopropylamino)pyridin-4-yl)methyl)- 5,5-dimethylimidazolidine-2,4-dione |
| 97 | 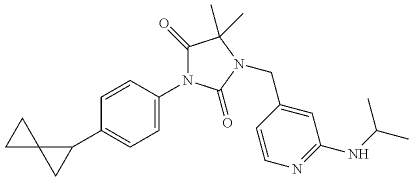 [SMILES: CC(C)Nc1cc(CN2C(=O)N(c3ccc(C4CC45CC5)cc3)C(=O)C2(C)C)ccn1] | 1-((2-(isopropylamino)pyridin-4-yl)methyl)- 5,5-dimethyl-3-(4-(spiro[2.2]pentan-1- yl)phenyl)imidazolidine-2,4-dione |
| 98 | 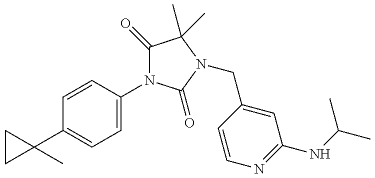 [SMILES: CC(C)Nc1cc(CN2C(=O)N(c3ccc(C4(C)CC4)cc3)C(=O)C2(C)C)ccn1] | 1-((2-(isopropylamino)pyridin-4-yl)methyl)- 5,5-dimethyl-3-(4-(1- methylcyclopropyl)phenyl)imidazolidine- 2,4-dione |
| 99 | 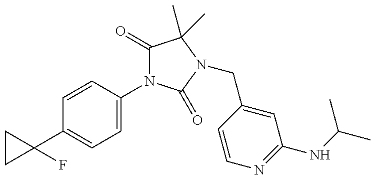 [SMILES: CC(C)Nc1cc(CN2C(=O)N(c3ccc(C4(F)CC4)cc3)C(=O)C2(C)C)ccn1] | 3-(4-(1-fluorocyclopropyl)phenyl)-1-((2- (isopropylamino)pyridin-4-yl)methyl)-5,5- dimethylimidazolidine-2,4-dione |
| 100 | 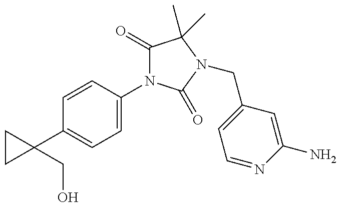 [SMILES: CC1(C)C(=O)N(c2ccc(C3(CO)CC3)cc2)C(=O)N1Cc1ccnc(N)c1] | 1-((2-aminopyridin-4-yl)methyl)-3-(4-(1- (hydroxymethyl)cyclopropyl)phenyl)-5,5- dimethylimidazolidine-2,4-dione |
| 101 | 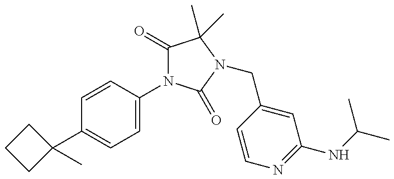 [SMILES: CC(C)Nc1cc(CN2C(=O)N(c3ccc(C4(C)CCC4)cc3)C(=O)C2(C)C)ccn1] | 1-((2-(isopropylamino)pyridin-4-yl)methyl)- 5,5-dimethyl-3-(4-(1- methylcyclobutyl)phenyl)imidazolidine-2,4- dione |
| 102 | 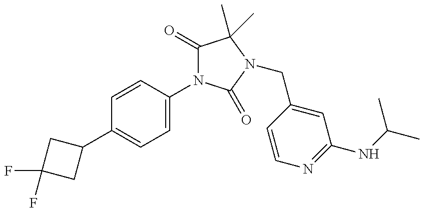 [SMILES: CC(C)Nc1cc(CN2C(=O)N(c3ccc(C4CC(F)(F)C4)cc3)C(=O)C2(C)C)ccn1] | 3-(4-(3,3-difluorocyclobutyl)phenyl)-1-((2- (isopropylamino)pyridin-4-yl)methyl)-5,5- dimethylimidazolidine-2,4-dione |
| 103 | 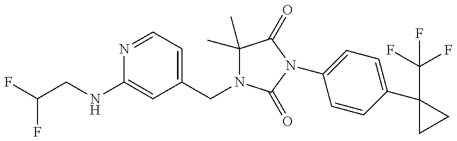 [SMILES: CC1(C)C(=O)N(c2ccc(C3(C(F)(F)F)CC3)cc2)C(=O)N1Cc1ccnc(NCC(F)F)c1] | 1-((2-((2,2-difluoroethyl)amino)pyridin-4- yl)methyl)-5,5-dimethyl-3-(4-(1- (trifluoromethyl)cyclopropyl)phenyl) imidazolidine-2,4-dione |
| 104 | 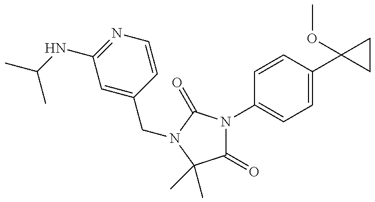 [SMILES: COC1(c2ccc(N3C(=O)N(Cc4ccnc(NC(C)C)c4)C(C)(C)C3=O)cc2)CC1] | 1-((2-(isopropylamino)pyridin-4-yl)methyl)- 3-(4-(1-methoxycyclopropyl)phenyl)-5,5- dimethylimidazolidine-2,4-dione |
| 105 | 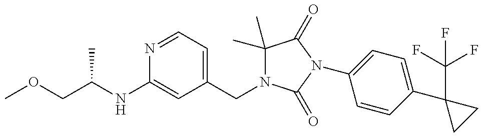 [SMILES: COC[C@H](C)Nc1cc(CN2C(=O)N(c3ccc(C4(C(F)(F)F)CC4)cc3)C(=O)C2(C)C)ccn1] | (S)-1-((2-((1-methoxypropan-2- yl)amino)pyridin-4-yl)methyl)-5,5-dimethyl- 3-(4-(1- (trifluoromethyl)cyclopropyl)phenyl) imidazolidine-2,4-dione |
| 106 | 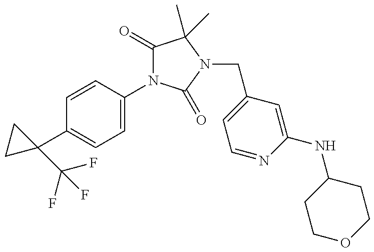 [SMILES: CC1(C)C(=O)N(c2ccc(C3(C(F)(F)F)CC3)cc2)C(=O)N1Cc1ccnc(NC2CCOCC2)c1] | 5,5-dimethyl-1-((2-((tetrahydro-2H-pyran-4- yl)amino)pyridin-4-yl)methyl)-3-(4-(1- (trifluoromethyl)cyclopropyl)phenyl) imidazolidine-2,4-dione |
| 107 | 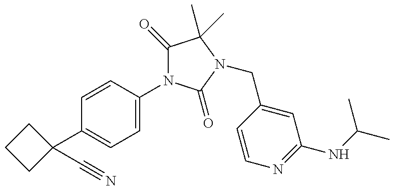 [SMILES: CC(C)Nc1cc(CN2C(=O)N(c3ccc(C4(C#N)CCC4)cc3)C(=O)C2(C)C)ccn1] | 1-(4-(3-((2-(isopropylamino)pyridin-4- yl)methyl)-4,4-dimethyl-2,5- dioxoimidazolidin-1-yl)phenyl)cyclobutane- 1-carbonitrile |
| 108 | 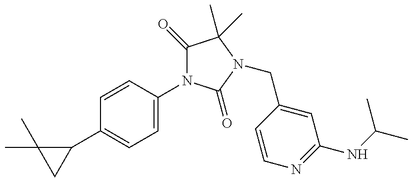 [SMILES: CC(C)Nc1cc(CN2C(=O)N(c3ccc(C4CC4(C)C)cc3)C(=O)C2(C)C)ccn1] | 3-(4-(2,2-dimethylcyclopropyl)phenyl)-1- ((2-(isopropylamino)pyridin-4-yl)methyl)- 5,5-dimethylimidazolidine-2,4-dione |
| 109 | 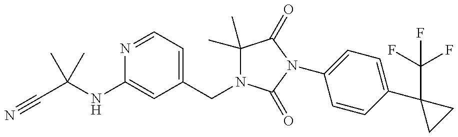 [SMILES: CC(C)(C#N)Nc1cc(CN2C(=O)N(c3ccc(C4(C(F)(F)F)CC4)cc3)C(=O)C2(C)C)ccn1] | 2-((4-((5,5-dimethyl-2,4-dioxo-3-(4-(1- (trifluoromethyl)cyclopropyl)phenyl) imidazolidin-1-yl)methyl) pyridin-2-yl)amino)-2-methylpropanenitrile |
| 110 | 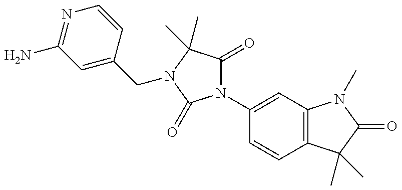 [SMILES: CN1C(=O)C(C)(C)c2ccc(N3C(=O)N(Cc4ccnc(N)c4)C(C)(C)C3=O)cc21] | 1-((2-aminopyridin-4-yl)methyl)-5,5- dimethyl-3-(1,3,3-trimethyl-2-oxoindolin-6- yl)imidazolidine-2,4-dione |
| 111 | 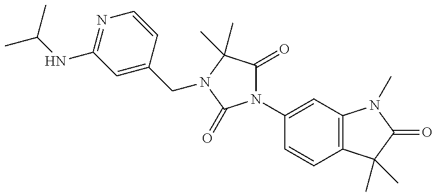 [SMILES: CC(C)Nc1cc(CN2C(=O)N(c3ccc4c(c3)N(C)C(=O)C4(C)C)C(=O)C2(C)C)ccn1] | 1-((2-(isopropylamino)pyridin-4-yl)methyl)- 5,5-dimethyl-3-(1,3,3-trimethyl-2- oxoindolin-6-yl)imidazolidine-2,4-dione |
| 112 | 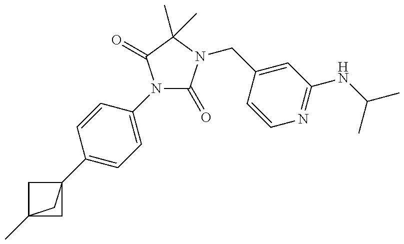 [SMILES: CC(C)Nc1cc(CN2C(=O)N(c3ccc(C45CC(C)(C4)C5)cc3)C(=O)C2(C)C)ccn1] | 1-((2-(isopropylamino)pyridin-4-yl)methyl)- 5,5-dimethyl-3-(4-(3- methylbicyclo[1.1.1]pentan-1- yl)phenyl)imidazolidine-2,4-dione |
| 113 | 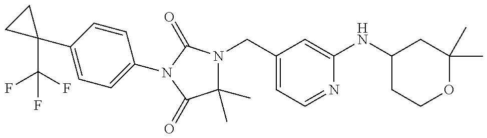 [SMILES: CC1(C)CC(Nc2cc(CN3C(=O)N(c4ccc(C5(C(F)(F)F)CC5)cc4)C(=O)C3(C)C)ccn2)CCO1] | 1-((2-((2,2-dimethyltetrahydro-2H-pyran-4- yl)amino)pyridin-4-yl)methyl)-5,5-dimethyl- 3-(4-(1- (trifluoromethyl)cyclopropyl)phenyl) imidazolidine-2,4-dione |
| 114 | 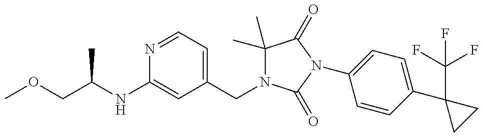 [SMILES: COC[C@@H](C)Nc1cc(CN2C(=O)N(c3ccc(C4(C(F)(F)F)CC4)cc3)C(=O)C2(C)C)ccn1] | (R)-1-((2-((1-methoxypropan-2- yl)amino)pyridin-4-yl)methyl)-5,5-dimethyl- 3-(4-(1- (trifluoromethyl)cyclopropyl)phenyl) imidazolidine-2,4-dione |
| 115 | 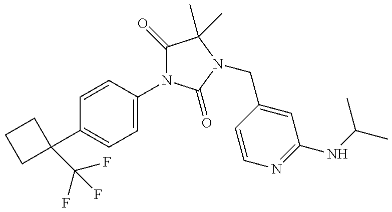 [SMILES: CC(C)Nc1cc(CN2C(=O)N(c3ccc(C4(C(F)(F)F)CCC4)cc3)C(=O)C2(C)C)ccn1] | 1-((2-(isopropylamino)pyridin-4-yl)methyl)- 5,5-dimethyl-3-(4-(1- (trifluoromethyl)cyclobutyl)phenyl) imidazolidine-2,4-dione |
| 116 | 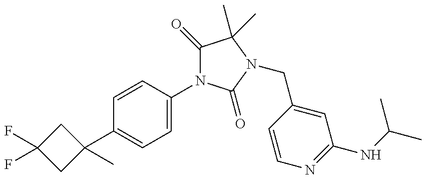 [SMILES: CC(C)Nc1cc(CN2C(=O)N(c3ccc(C4(C)CC(F)(F)C4)cc3)C(=O)C2(C)C)ccn1] | 3-(4-(3,3-difluoro-1- methylcyclobutyl)phenyl)-1-((2- (isopropylamino)pyridin-4-yl)methyl)-5,5- dimethylimidazolidine-2,4-dione |
| 117 | 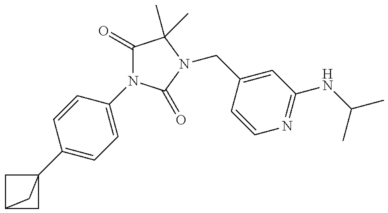 [SMILES: CC(C)Nc1cc(CN2C(=O)N(c3ccc(C45CC(C4)C5)cc3)C(=O)C2(C)C)ccn1] | 3-(4-(bicyclo[1.1.1]pentan-1-yl)phenyl)-1- ((2-(isopropylamino)pyridin-4-yl)methyl)- 5,5-dimethylimidazolidine-2,4-dione |
| 118 | 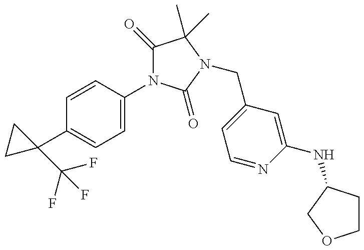 [SMILES: CC1(C)C(=O)N(c2ccc(C3(C(F)(F)F)CC3)cc2)C(=O)N1Cc1ccnc(N[C@@H]2CCOC2)c1] | (R)-5,5-dimethyl-1-((2-((tetrahydrofuran-3- yl)amino)pyridin-4-yl)methyl)-3-(4-(1- (trifluoromethyl)cyclopropyl)phenyl) imidazolidine-2,4-dione |
| 119 | 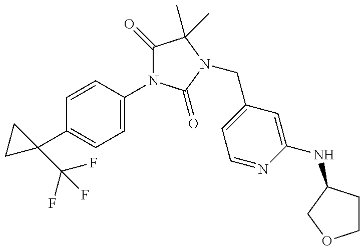 [SMILES: CC1(C)C(=O)N(c2ccc(C3(C(F)(F)F)CC3)cc2)C(=O)N1Cc1ccnc(N[C@H]2CCOC2)c1] | (S)-5,5-dimethyl-1-((2-((tetrahydrofuran-3- yl)amino)pyridin-4-yl)methyl)-3-(4-(1- (trifluoromethyl)cyclopropyl)phenyl) imidazolidine-2,4-dione |
| 120 | 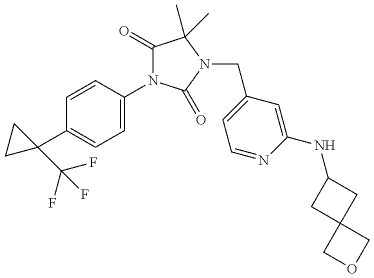 [SMILES: CC1(C)C(=O)N(c2ccc(C3(C(F)(F)F)CC3)cc2)C(=O)N1Cc1ccnc(NC2CC3(COC3)C2)c1] | 1-((2-((2-oxaspiro[3.3]heptan-6- yl)amino)pyridin-4-yl)methyl)-5,5-dimethyl- 3-(4-(1- (trifluoromethyl)cyclopropyl)phenyl) imidazolidine-2,4-dione |
| 121 | 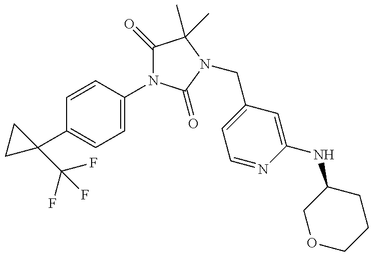 [SMILES: CC1(C)C(=O)N(c2ccc(C3(C(F)(F)F)CC3)cc2)C(=O)N1Cc1ccnc(N[C@H]2CCCOC2)c1] | (S)-5,5-dimethyl-1-((2-((tetrahydro-2H- pyran-3-yl)amino)pyridin-4-yl)methyl)-3-(4- (1- (trifluoromethyl)cyclopropyl)phenyl) imidazolidine-2,4-dione |
| 122 | 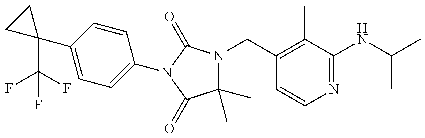 [SMILES: Cc1c(CN2C(=O)N(c3ccc(C4(C(F)(F)F)CC4)cc3)C(=O)C2(C)C)ccnc1NC(C)C] | 1-((2-(isopropylamino)-3-methylpyridin-4- yl)methyl)-5,5-dimethyl-3-(4-(1- (trifluoromethyl)cyclopropyl)phenyl) imidazolidine-2,4-dione |
| 123 | 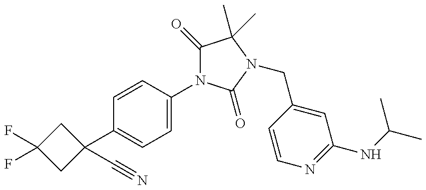 [SMILES: CC(C)Nc1cc(CN2C(=O)N(c3ccc(C4(C#N)CC(F)(F)C4)cc3)C(=O)C2(C)C)ccn1] | 3,3-difluoro-1-(4-(3-((2- (isopropylamino)pyridin-4-yl)methyl)-4,4- dimethyl-2,5-dioxoimidazolidin-1- yl)phenyl)cyclobutane-1-carbonitrile |
| 124 | 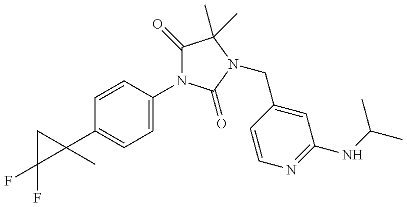 [SMILES: CC(C)Nc1cc(CN2C(=O)N(c3ccc(C4(C)CC4(F)F)cc3)C(=O)C2(C)C)ccn1] | 3-(4-(2,2-difluoro-1- methylcyclopropyl)phenyl)-1-((2- (isopropylamino)pyridin-4-yl)methyl)-5,5- dimethylimidazolidine-2,4-dione |
| 125 | 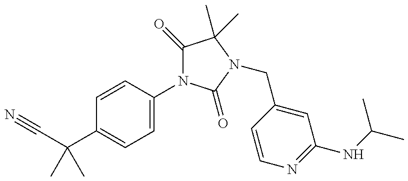 [SMILES: CC(C)Nc1cc(CN2C(=O)N(c3ccc(C(C)(C)C#N)cc3)C(=O)C2(C)C)ccn1] | 2-(4-(3-((2-(isopropylamino)pyridin-4- yl)methyl)-4,4-dimethyl-2,5- dioxoimidazolidin-1-yl)phenyl)-2- methylpropanenitrile |
| 126 | 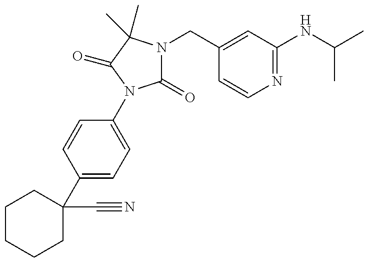 [SMILES: CC(C)Nc1cc(CN2C(=O)N(c3ccc(C4(C#N)CCCCC4)cc3)C(=O)C2(C)C)ccn1] | 1-(4-(3-((2-(isopropylamino)pyridin-4- yl)methyl)-4,4-dimethyl-2,5- dioxoimidazolidin-1-yl)phenyl)cyclohexane- 1-carbonitrile |
| 127 | 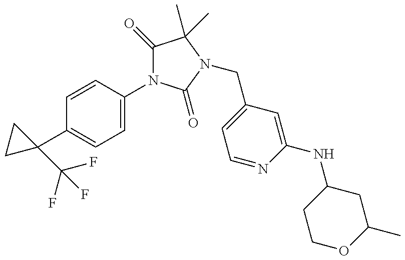 [SMILES: CC1CC(Nc2cc(CN3C(=O)N(c4ccc(C5(C(F)(F)F)CC5)cc4)C(=O)C3(C)C)ccn2)CCO1] | 5,5-dimethyl-1-((2-((2-methyltetrahydro-2H- pyran-4-yl)amino)pyridin-4-yl)methyl)-3-(4- (1- (trifluoromethyl)cyclopropyl)phenyl) imidazolidine-2,4-dione |
| 128 | 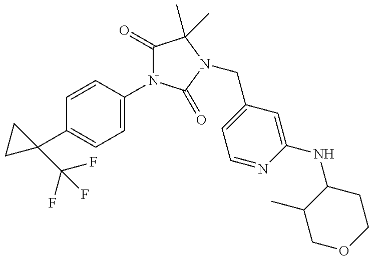 [SMILES: CC1COCCC1Nc1cc(CN2C(=O)N(c3ccc(C4(C(F)(F)F)CC4)cc3)C(=O)C2(C)C)ccn1] | 5,5-dimethyl-1-((2-((3-methyltetrahydro-2H- pyran-4-yl)amino)pyridin-4-yl)methyl)-3-(4- (1- (trifluoromethyl)cyclopropyl)phenyl) imidazolidine-2,4-dione |
| 129 | 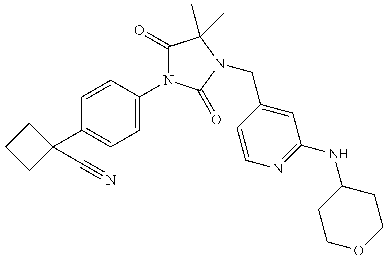 [SMILES: CC1(C)C(=O)N(c2ccc(C3(C#N)CCC3)cc2)C(=O)N1Cc1ccnc(NC2CCOCC2)c1] | 1-(4-(4,4-dimethyl-2,5-dioxo-3-((2- ((tetrahydro-2H-pyran-4-yl)amino)pyridin-4- yl)methyl)imidazolidin-1- yl)phenyl)cyclobutane-1-carbonitrile |
| 130 | 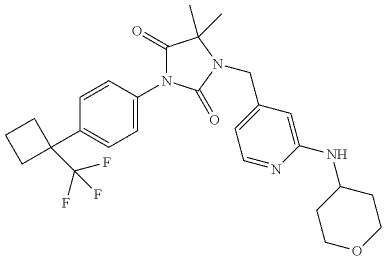 [SMILES: CC1(C)C(=O)N(c2ccc(C3(C(F)(F)F)CCC3)cc2)C(=O)N1Cc1ccnc(NC2CCOCC2)c1] | 5,5-dimethyl-1-((2-((tetrahydro-2H-pyran-4- yl)amino)pyridin-4-yl)methyl)-3-(4-(1- (trifluoromethyl)cyclobutyl)phenyl) imidazolidine-2,4-dione |
| 131 | 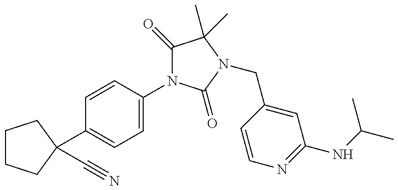 [SMILES: CC(C)Nc1cc(CN2C(=O)N(c3ccc(C4(C#N)CCCC4)cc3)C(=O)C2(C)C)ccn1] | 1-(4-(3-((2-(isopropylamino)pyridin-4- yl)methyl)-4,4-dimethyl-2,5- dioxoimidazolidin-1- yl)phenyl)cyclopentane-1-carbonitrile |
| 132 | 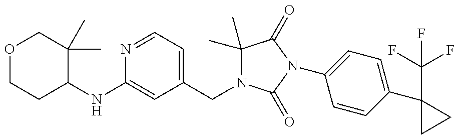 [SMILES: CC1(C)COCCC1Nc1cc(CN2C(=O)N(c3ccc(C4(C(F)(F)F)CC4)cc3)C(=O)C2(C)C)ccn1] | 1-((2-((3,3-dimethyltetrahydro-2H-pyran-4- yl)amino)pyridin-4-yl)methyl)-5,5-dimethyl- 3-(4-(1- (trifluoromethyl)cyclopropyl)phenyl) imidazolidine-2,4-dione |
| 133 | 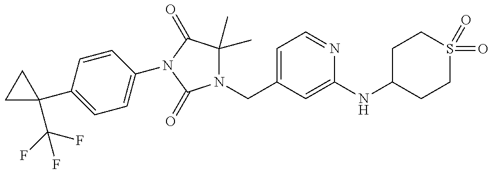 [SMILES: CC1(C)C(=O)N(c2ccc(C3(C(F)(F)F)CC3)cc2)C(=O)N1Cc1ccnc(NC2CCS(=O)(=O)CC2)c1] | 1-((2-((1,1-dioxidotetrahydro-2H-thiopyran- 4-yl)amino)pyridin-4-yl)methyl)-5,5- dimethyl-3-(4-(1- (trifluoromethyl)cyclopropyl)phenyl) imidazolidine-2,4-dione |
| 134 | 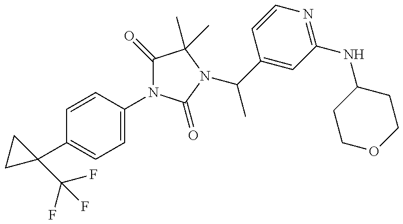 [SMILES: CC(c1ccnc(NC2CCOCC2)c1)N1C(=O)N(c2ccc(C3(C(F)(F)F)CC3)cc2)C(=O)C1(C)C] | 5,5-dimethyl-1-(1-(2-((tetrahydro-2H-pyran- 4-yl)amino)pyridin-4-yl)ethyl)-3-(4-(1- (trifluoromethyl)cyclopropyl)phenyl) imidazolidine-2,4-dione |
| 135 | 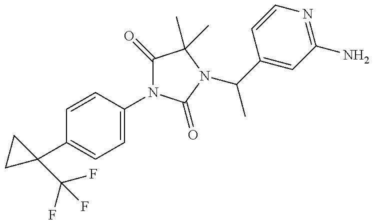 [SMILES: CC(c1ccnc(N)c1)N1C(=O)N(c2ccc(C3(C(F)(F)F)CC3)cc2)C(=O)C1(C)C] | 1-(1-(2-aminopyridin-4-yl)ethyl)-5,5- dimethyl-3-(4-(1- (trifluoromethyl)cyclopropyl)phenyl) imidazolidine-2,4-dione |
| 136 | 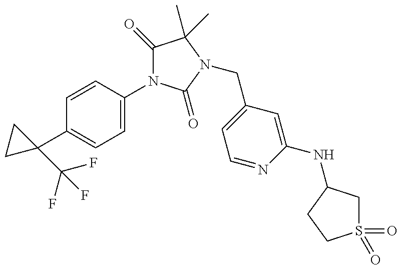 [SMILES: CC1(C)C(=O)N(c2ccc(C3(C(F)(F)F)CC3)cc2)C(=O)N1Cc1ccnc(NC2CCS(=O)(=O)C2)c1] | 1-((2-((1,1-dioxidotetrahydrothiophen-3- yl)amino)pyridin-4-yl)methyl)-5,5-dimethyl- 3-(4-(1- (trifluoromethyl)cyclopropyl)phenyl) imidazolidine-2,4-dione |
| 137 | 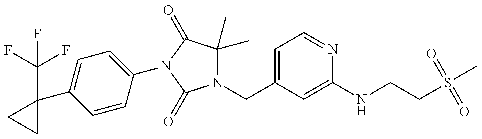 [SMILES: CC1(C)C(=O)N(c2ccc(C3(C(F)(F)F)CC3)cc2)C(=O)N1Cc1ccnc(NCCS(C)(=O)=O)c1] | 5,5-dimethyl-1-((2-((2- (methylsulfonyl)ethyl)amino)pyridin-4- yl)methyl)-3-(4-(1- (trifluoromethyl)cyclopropyl)phenyl) imidazolidine-2,4-dione |
| 138 | 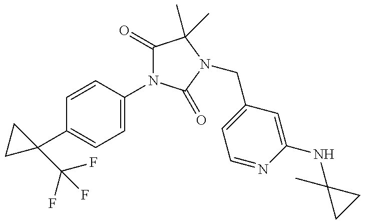 [SMILES: CC1(Nc2cc(CN3C(=O)N(c4ccc(C5(C(F)(F)F)CC5)cc4)C(=O)C3(C)C)ccn2)CC1] | 5,5-dimethyl-1-((2-((1- methylcyclopropyl)amino)pyridin-4- yl)methyl)-3-(4-(1- (trifluoromethyl)cyclopropyl)phenyl) imidazolidine-2,4-dione |
| 139 | 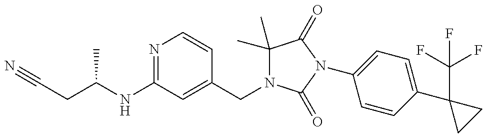 [SMILES: C[C@@H](CC#N)Nc1cc(CN2C(=O)N(c3ccc(C4(C(F)(F)F)CC4)cc3)C(=O)C2(C)C)ccn1] | (S)-3-((4-((5,5-dimethyl-2,4-dioxo-3-(4-(1- (trifluoromethyl)cyclopropyl)phenyl) imidazolidin-1-yl)methyl)pyridin-2- yl)amino)butanenitrile |
| 140 | 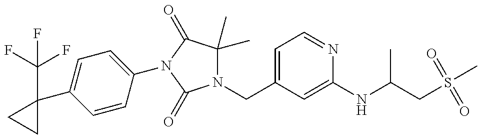 [SMILES: CC(CS(C)(=O)=O)Nc1cc(CN2C(=O)N(c3ccc(C4(C(F)(F)F)CC4)cc3)C(=O)C2(C)C)ccn1] | 5,5-dimethyl-1-((2-((1- (methylsulfonyl)propan-2-yl)amino)pyridin- 4-yl)methyl)-3-(4-(1- (trifluoromethyl)cyclopropyl)phenyl) imidazolidine-2,4-dione |
| 141 | 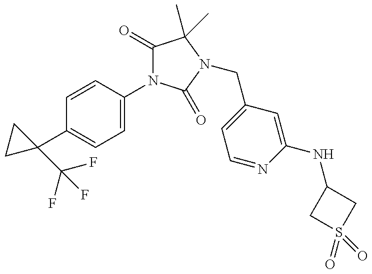 [SMILES: CC1(C)C(=O)N(c2ccc(C3(C(F)(F)F)CC3)cc2)C(=O)N1Cc1ccnc(NC2CS(=O)(=O)C2)c1] | 1-((2-((1,1-dioxidothietan-3- yl)amino)pyridin-4-yl)methyl)-5,5-dimethyl- 3-(4-(1- (trifluoromethyl)cyclopropyl)phenyl) imidazolidine-2,4-dione |
| 142 | 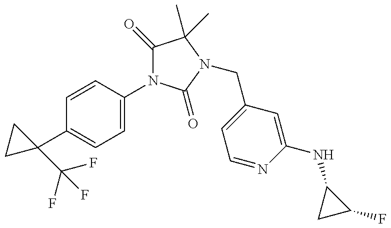 [SMILES: CC1(C)C(=O)N(c2ccc(C3(C(F)(F)F)CC3)cc2)C(=O)N1Cc1ccnc(N[C@H]2C[C@H]2F)c1] | 1-((2-(((1S,2R)-2- fluorocyclopropyl)amino)pyridin-4- yl)methyl)-5,5-dimethyl-3-(4-(1- (trifluoromethyl)cyclopropyl)phenyl) imidazolidine-2,4-dione |
| 143 | 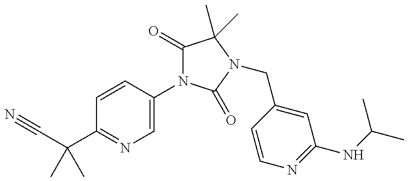 [SMILES: CC(C)Nc1cc(CN2C(=O)N(c3ccc(C(C)(C)C#N)nc3)C(=O)C2(C)C)ccn1] | 2-(5-(3-((2-(isopropylamino)pyridin-4- yl)methyl)-4,4-dimethyl-2,5- dioxoimidazolidin-1-yl)pyridin-2-yl)-2- methylpropanenitrile |
| 144 | 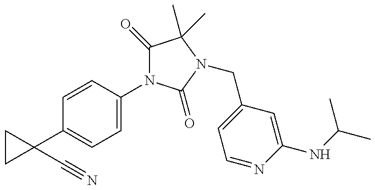 [SMILES: CC(C)Nc1cc(CN2C(=O)N(c3ccc(C4(C#N)CC4)cc3)C(=O)C2(C)C)ccn1] | 1-(4-(3-((2-(isopropylamino)pyridin-4- yl)methyl)-4,4-dimethyl-2,5- dioxoimidazolidin-1- yl)phenyl)cyclopropane-1-carbonitrile |
| 145 | 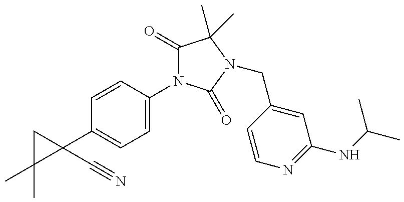 [SMILES: CC(C)Nc1cc(CN2C(=O)N(c3ccc(C4(C#N)CC4(C)C)cc3)C(=O)C2(C)C)ccn1] | 1-(4-(3-((2-(isopropylamino)pyridin-4- yl)methyl)-4,4-dimethyl-2,5- dioxoimidazolidin-1-yl)phenyl)-2,2- dimethylcyclopropane-1-carbonitrile |
| 146 | 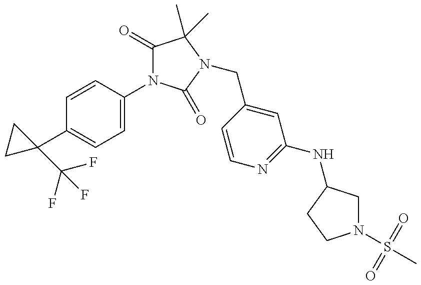 [SMILES: CC1(C)C(=O)N(c2ccc(C3(C(F)(F)F)CC3)cc2)C(=O)N1Cc1ccnc(NC2CCN(S(C)(=O)=O)C2)c1] | 5,5-dimethyl-1-((2-((1- (methylsulfonyl)pyrrolidin-3- yl)amino)pyridin-4-yl)methyl)-3-(4-(1- (trifluoromethyl)cyclopropyl)phenyl) imidazolidine-2,4-dione |
| 147 | 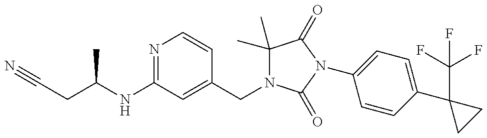 [SMILES: C[C@H](CC#N)Nc1cc(CN2C(=O)N(c3ccc(C4(C(F)(F)F)CC4)cc3)C(=O)C2(C)C)ccn1] | (R)-3-((4-((5,5-dimethyl-2,4-dioxo-3-(4-(1- (trifluoromethyl)cyclopropyl)phenyl) imidazolidin-1-yl)methyl)pyridin-2- yl)amino)butanenitrile |
| 148 | 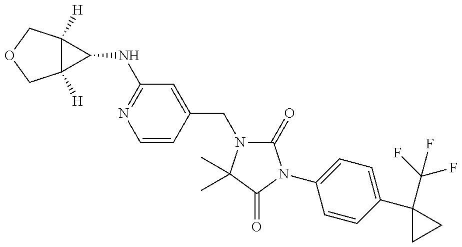 [SMILES: CC1(C)C(=O)N(c2ccc(C3(C(F)(F)F)CC3)cc2)C(=O)N1Cc1ccnc(N[C@H]2[C@@H]3COC[C@@H]32)c1] | 1-((2-(((1R,5S,6r)-3- oxabicyclo[3.1.0]hexan-6-yl)amino)pyridin- 4-yl)methyl)-5,5-dimethyl-3-(4-(1- (trifluoromethyl)cyclopropyl)phenyl) imidazolidine-2,4-dione |
| 149 | 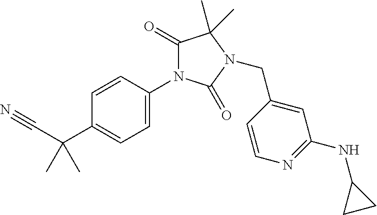 [SMILES: CC(C)(C#N)c1ccc(N2C(=O)N(Cc3ccnc(NC4CC4)c3)C(C)(C)C2=O)cc1] | 2-(4-(3-((2-(cyclopropylamino)pyridin-4- yl)methyl)-4,4-dimethyl-2,5- dioxoimidazolidin-1-yl)phenyl)-2- methylpropanenitrile |
| 150 | 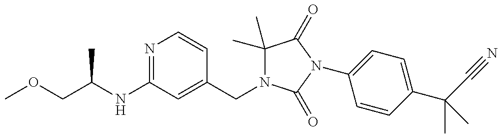 [SMILES: COC[C@@H](C)Nc1cc(CN2C(=O)N(c3ccc(C(C)(C)C#N)cc3)C(=O)C2(C)C)ccn1] | (R)-2-(4-(3-((2-((1-methoxypropan-2- yl)amino)pyridin-4-yl)methyl)-4,4-dimethyl- 2,5-dioxoimidazolidin-1-yl)phenyl)-2- methylpropanenitrile |
| 151 | 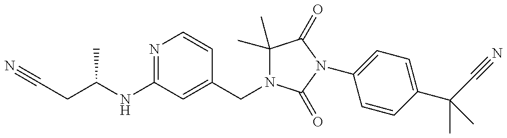 [SMILES: C[C@@H](CC#N)Nc1cc(CN2C(=O)N(c3ccc(C(C)(C)C#N)cc3)C(=O)C2(C)C)ccn1] | (S)-3-((4-((3-(4-(2-cyanopropan-2- yl)phenyl)-5,5-dimethyl-2,4- dioxoimidazolidin-1-yl)methyl)pyridin-2- yl)amino)butanenitrile |
| 152 | 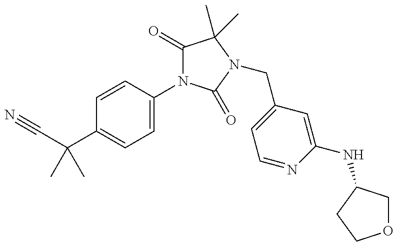 [SMILES: CC(C)(C#N)c1ccc(N2C(=O)N(Cc3ccnc(N[C@H]4CCOC4)c3)C(C)(C)C2=O)cc1] | (S)-2-(4-(4,4-dimethyl-2,5-dioxo-3-((2- ((tetrahydrofuran-3-yl)amino)pyridin-4- yl)methyl)imidazolidin-1-yl)phenyl)-2- methylpropanenitrile |
| 153 | 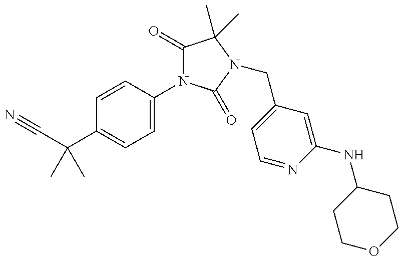 [SMILES: CC(C)(C#N)c1ccc(N2C(=O)N(Cc3ccnc(NC4CCOCC4)c3)C(C)(C)C2=O)cc1] | 2-(4-(4,4-dimethyl-2,5-dioxo-3-((2- ((tetrahydro-2H-pyran-4-yl)amino)pyridin-4- yl)methyl)imidazolidin-1-yl)phenyl)-2- methylpropanenitrile |
| 154 | 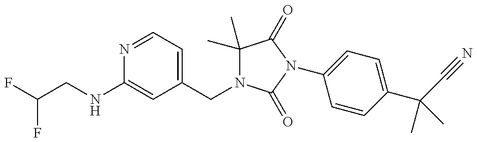 [SMILES: CC(C)(C#N)c1ccc(N2C(=O)N(Cc3ccnc(NCC(F)F)c3)C(C)(C)C2=O)cc1] | 2-(4-(3-((2-((2,2- difluoroethyl)amino)pyridin-4-yl)methyl)- 4,4-dimethyl-2,5-dioxoimidazolidin-1- yl)phenyl)-2-methylpropanenitrile |
| 155 | 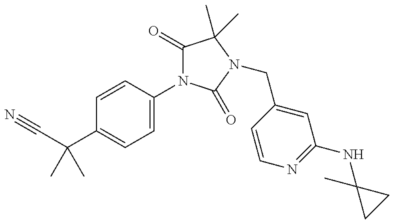 [SMILES: CC1(Nc2cc(CN3C(=O)N(c4ccc(C(C)(C)C#N)cc4)C(=O)C3(C)C)ccn2)CC1] | 2-(4-(4,4-dimethyl-3-((2-((1- methylcyclopropyl)amino)pyridin-4- yl)methyl)-2,5-dioxoimidazolidin-1- yl)phenyl)-2-methylpropanenitrile |
| 156 | 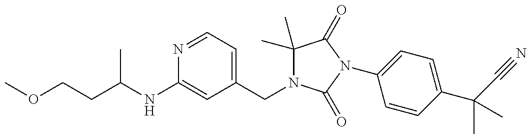 [SMILES: COCCC(C)Nc1cc(CN2C(=O)N(c3ccc(C(C)(C)C#N)cc3)C(=O)C2(C)C)ccn1] | 2-(4-(3-((2-((4-methoxybutan-2- yl)amino)pyridin-4-yl)methyl)-4,4-dimethyl- 2,5-dioxoimidazolidin-1-yl)phenyl)-2- methylpropanenitrile |
| 157 | 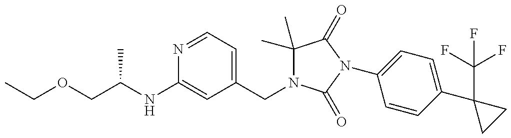 [SMILES: CCOC[C@H](C)Nc1cc(CN2C(=O)N(c3ccc(C4(C(F)(F)F)CC4)cc3)C(=O)C2(C)C)ccn1] | (S)-1-((2-((1-ethoxypropan-2- yl)amino)pyridin-4-yl)methyl)-5,5-dimethyl- 3-(4-(1- (trifluoromethyl)cyclopropyl)phenyl) imidazolidine-2,4-dione |
| 158 | 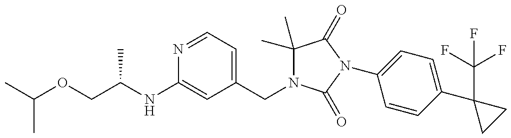 [SMILES: CC(C)OC[C@H](C)Nc1cc(CN2C(=O)N(c3ccc(C4(C(F)(F)F)CC4)cc3)C(=O)C2(C)C)ccn1] | (S)-1-((2-((1-isopropoxypropan-2- yl)amino)pyridin-4-yl)methyl)-5,5-dimethyl- 3-(4-(1- (trifluoromethyl)cyclopropyl)phenyl) imidazolidine-2,4-dione |
| 159 | 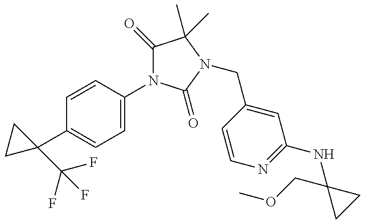 [SMILES: COCC1(Nc2cc(CN3C(=O)N(c4ccc(C5(C(F)(F)F)CC5)cc4)C(=O)C3(C)C)ccn2)CC1] | 1-((2-((1- (methoxymethyl)cyclopropyl)amino)pyridin- 4-yl)methyl)-5,5-dimethyl-3-(4-(1- (trifluoromethyl)cyclopropyl)phenyl) imidazolidine-2,4-dione |
| 160 | 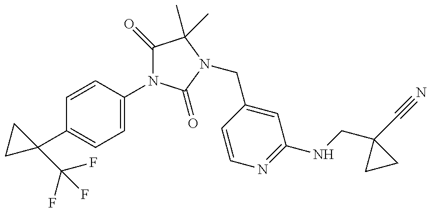 [SMILES: CC1(C)C(=O)N(c2ccc(C3(C(F)(F)F)CC3)cc2)C(=O)N1Cc1ccnc(NCC2(C#N)CC2)c1] | 1-(((4-((5,5-dimethyl-2,4-dioxo-3-(4-(1- (trifluoromethyl)cyclopropyl)phenyl) imidazolidin-1-yl)methyl)pyridin-2- yl)amino)methyl)cyclopropane-1- carbonitrile |
| 161 | 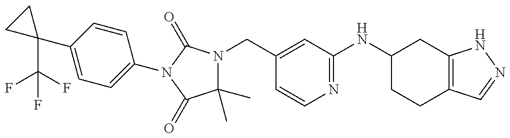 [SMILES: CC1(C)C(=O)N(c2ccc(C3(C(F)(F)F)CC3)cc2)C(=O)N1Cc1ccnc(NC2CCc3cn[nH]c3C2)c1] | 5,5-dimethyl-1-((2-((4,5,6,7-tetrahydro-1H- indazol-6-yl)amino)pyridin-4-yl)methyl)-3- (4-(1- (trifluoromethyl)cyclopropyl)phenyl) imidazolidine-2,4-dione |
| 162 | 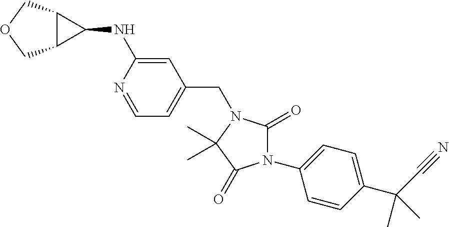 [SMILES: CC(C)(C#N)c1ccc(N2C(=O)N(Cc3ccnc(N[C@H]4[C@@H]5COC[C@@H]54)c3)C(C)(C)C2=O)cc1] | 2-(4-(3-((2-(((1R,5S,6r)-3- oxabicyclo[3.1.0]hexan-6-yl)amino)pyridin- 4-yl)methyl)-4,4-dimethyl-2,5- dioxoimidazolidin-1-yl)phenyl)-2- methylpropanenitrile |
| 163 | 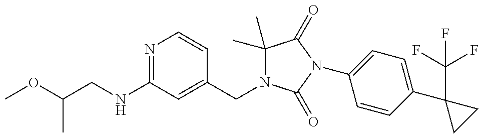 [SMILES: COC(C)CNc1cc(CN2C(=O)N(c3ccc(C4(C(F)(F)F)CC4)cc3)C(=O)C2(C)C)ccn1] | 1-((2-((2-methoxypropyl)amino)pyridin-4- yl)methyl)-5,5-dimethyl-3-(4-(1- (trifluoromethyl)cyclopropyl)phenyl) imidazolidine-2,4-dione |
| 164 | 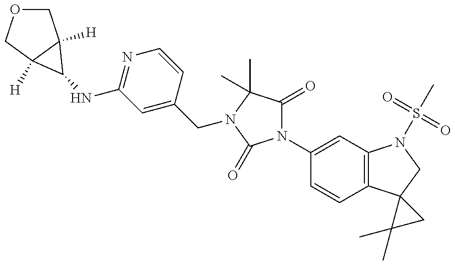 [SMILES: CC1(C)C(=O)N(c2ccc3c(c2)N(S(C)(=O)=O)CC32CC2(C)C)C(=O)N1Cc1ccnc(N[C@H]2[C@@H]3COC[C@@H]32)c1] | 1-((2-(((1R,5S,6r)-3- oxabicyclo[3.1.0]hexan-6-yl)amino)pyridin- 4-yl)methyl)-3-(2,2-dimethyl-1′- (methylsulfonyl)spiro[cyclopropane-1,3′- indolin]-6′-yl)-5,5-dimethylimidazolidine- 2,4-dione |
| 165 | 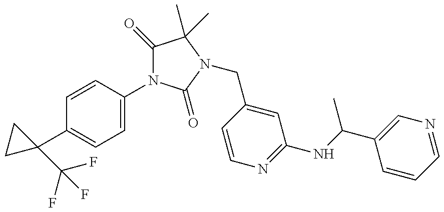 [SMILES: CC(Nc1cc(CN2C(=O)N(c3ccc(C4(C(F)(F)F)CC4)cc3)C(=O)C2(C)C)ccn1)c1cccnc1] | 5,5-dimethyl-1-((2-((1-(pyridin-3- ylethylamino)pyridin-4-yl)methyl)-3-(4-(1- (trifluoromethyl)cyclopropyl)phenyl) imidazolidine-2,4-dione |
| 166 | 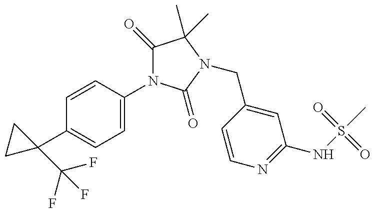 [SMILES: CC1(C)C(=O)N(c2ccc(C3(C(F)(F)F)CC3)cc2)C(=O)N1Cc1ccnc(NS(C)(=O)=O)c1] | N-(4-((5,5-dimethyl-2,4-dioxo-3-(4-(1- (trifluoromethyl)cyclopropyl)phenyl) imidazolidin-1-yl)methyl)pyridin-2- yl)methanesulfonamide |
| 167 | 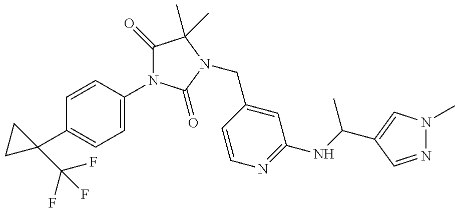 [SMILES: CC(Nc1cc(CN2C(=O)N(c3ccc(C4(C(F)(F)F)CC4)cc3)C(=O)C2(C)C)ccn1)c1cnn(C)c1] | 5,5-dimethyl-1-((2-((1-(1-methyl-1H- pyrazol-4-yl)ethyl)amino)pyridin-4- yl)methyl)-3-(4-(1- (trifluoromethyl)cyclopropyl)phenyl) imidazolidine-2,4-dione |
| 168 | 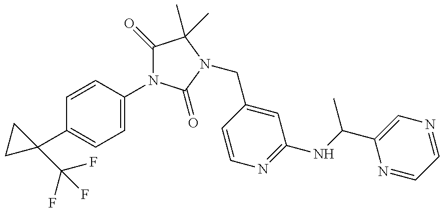 [SMILES: CC(Nc1cc(CN2C(=O)N(c3ccc(C4(C(F)(F)F)CC4)cc3)C(=O)C2(C)C)ccn1)c1cnccn1] | 5,5-dimethyl-1-((2-((1-(pyrazin-2- yl)ethyl)amino)pyridin-4-yl)methyl)-3-(4-(1- (trifluoromethyl)cyclopropyl)phenyl) imidazolidine-2,4-dione |
| 169 | 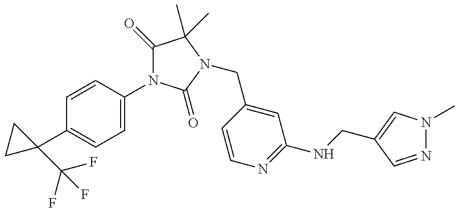 [SMILES: Cn1cc(CNc2cc(CN3C(=O)N(c4ccc(C5(C(F)(F)F)CC5)cc4)C(=O)C3(C)C)ccn2)cn1] | 5,5-dimethyl-1-((2-(((1-methyl-1H-pyrazol- 4-yl)methyl)amino)pyridin-4-yl)methyl)-3- (4-(1- (trifluoromethyl)cyclopropyl)phenyl) imidazolidine-2,4-dione |
| 170 | 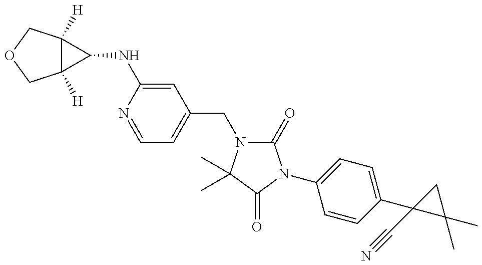 [SMILES: CC1(C)C(=O)N(c2ccc(C3(C#N)CC3(C)C)cc2)C(=O)N1Cc1ccnc(N[C@H]2[C@@H]3COC[C@@H]32)c1] | 1-(4-(3-((2-(((1R,5S,6r)-3- oxabicyclo[3.1.0]hexan-6-yl)amino)pyridin- 4-yl)methyl)-4,4-dimethyl-2,5- dioxoimidazolidin-1-yl)phenyl)-2,2- dimethylcyclopropane-1-carbonitrile |
| 171 | 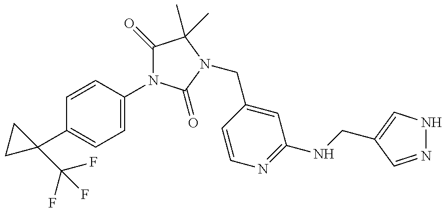 [SMILES: CC1(C)C(=O)N(c2ccc(C3(C(F)(F)F)CC3)cc2)C(=O)N1Cc1ccnc(NCc2cn[nH]c2)c1] | 1-((2-(((1H-pyrazol-4- yl)methyl)amino)pyridin-4-yl)methyl)-5,5- dimethyl-3-(4-(1- (trifluoromethyl)cyclopropyl)phenyl) imidazolidine-2,4-dione |
| 172 | 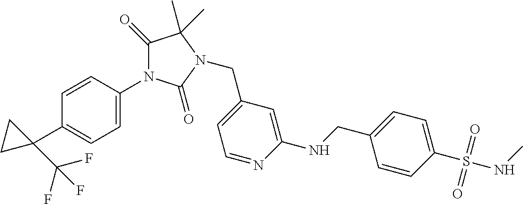 [SMILES: CNS(=O)(=O)c1ccc(CNc2cc(CN3C(=O)N(c4ccc(C5(C(F)(F)F)CC5)cc4)C(=O)C3(C)C)ccn2)cc1] | 4-(((4-((5,5-dimethyl-2,4-dioxo-3-(4-(1- (trifluoromethyl)cyclopropyl)phenyl) imidazolidin-1-yl)methyl)pyridin-2- yl)amino)methyl)-N- methylbenzenesulfonamide |
| 173 | 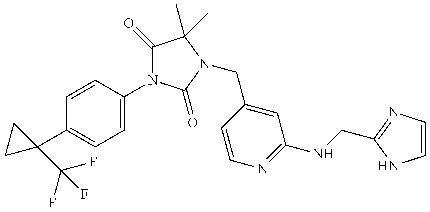 [SMILES: CC1(C)C(=O)N(c2ccc(C3(C(F)(F)F)CC3)cc2)C(=O)N1Cc1ccnc(NCc2ncc[nH]2)c1] | 1-((2-(((1H-imidazol-2- yl)methyl)amino)pyridin-4-yl)methyl)-5,5- dimethyl-3-(4-(1- (trifluoromethyl)cyclopropyl)phenyl) imidazolidine-2,4-dione |
| 174 | 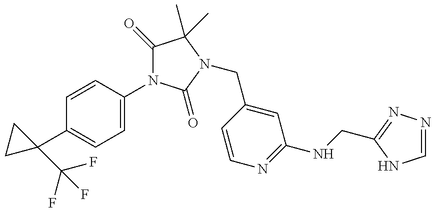 [SMILES: CC1(C)C(=O)N(c2ccc(C3(C(F)(F)F)CC3)cc2)C(=O)N1Cc1ccnc(NCc2nnc[nH]2)c1] | 1-((2-(((4H-1,2,4-triazol-3- yl)methyl)amino)pyridin-4-yl)methyl)-5,5- dimethyl-3-(4-(1- (trifluoromethyl)cyclopropyl)phenyl) imidazolidine-2,4-dione |
| 175 | 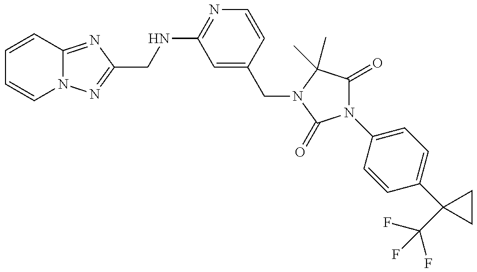 [SMILES: CC1(C)C(=O)N(c2ccc(C3(C(F)(F)F)CC3)cc2)C(=O)N1Cc1ccnc(NCc2nc3ccccn3n2)c1] | 1-((2-(([1,2,4]triazolo[1,5-a]pyridin-2- ylmethyl)amino)pyridin-4-yl)methyl)-5,5- dimethyl-3-(4-(1- (trifluoromethyl)cyclopropyl)phenyl) imidazolidine-2,4-dione |
| 176 | 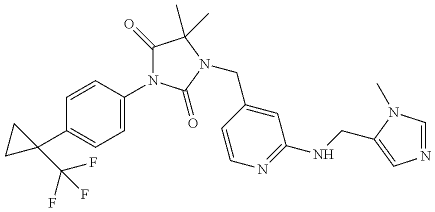 [SMILES: Cn1cncc1CNc1cc(CN2C(=O)N(c3ccc(C4(C(F)(F)F)CC4)cc3)C(=O)C2(C)C)ccn1] | 5,5-dimethyl-1-((2-(((1-methyl-1H-imidazol- 5-yl)methyl)amino)pyridin-4-yl)methyl)-3- (4-(1- (trifluoromethyl)cyclopropyl)phenyl) imidazolidine-2,4-dione |
| 177 | 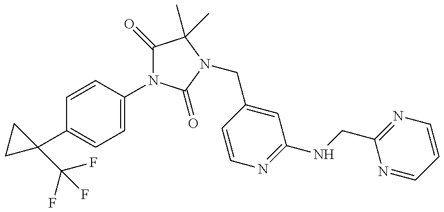 [SMILES: CC1(C)C(=O)N(c2ccc(C3(C(F)(F)F)CC3)cc2)C(=O)N1Cc1ccnc(NCc2ncccn2)c1] | 5,5-dimethyl-1-((2-((pyrimidin-2- ylmethyl)amino)pyridin-4-yl)methyl)-3-(4- (1- (trifluoromethyl)cyclopropyl)phenyl) imidazolidine-2,4-dione |
| 178 | 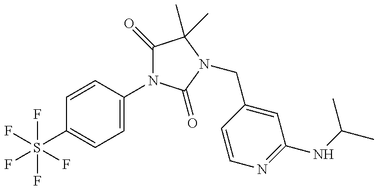 [SMILES: CC(C)Nc1cc(CN2C(=O)N(c3ccc(S(F)(F)(F)(F)F)cc3)C(=O)C2(C)C)ccn1] | 1-((2-(isopropylamino)pyridin-4-yl)methyl)- 5,5-dimethyl-3-(4-(pentafluoro-16- sulfaneyl)phenyl)imidazolidine-2,4-dione |
| 179 | 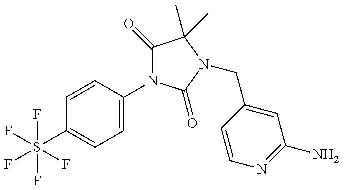 [SMILES: CC1(C)C(=O)N(c2ccc(S(F)(F)(F)(F)F)cc2)C(=O)N1Cc1ccnc(N)c1] | 1-((2-aminopyridin-4-yl)methyl)-5,5- dimethyl-3-(4-(pentafluoro-16- sulfaneyl)phenyl)imidazolidine-2,4-dione |
| 180 | 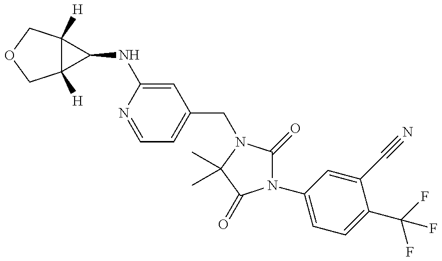 [SMILES: CC1(C)C(=O)N(c2ccc(C(F)(F)F)c(C#N)c2)C(=O)N1Cc1ccnc(N[C@H]2[C@@H]3COC[C@@H]32)c1] | 5-(3-((2-(((1R,5S,6r)-3- oxabicyclo[3.1.0]hexan-6-yl)amino)pyridin- 4-yl)methyl)-4,4-dimethyl-2,5- dioxoimidazolidin-1-yl)-2- (trifluoromethyl)benzonitrile |
| 181 | 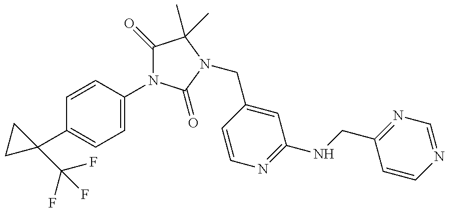 [SMILES: CC1(C)C(=O)N(c2ccc(C3(C(F)(F)F)CC3)cc2)C(=O)N1Cc1ccnc(NCc2ccncn2)c1] | 5,5-dimethyl-1-((2-((pyrimidin-4- ylmethyl)amino)pyridin-4-yl)methyl)-3-(4- (1- (trifluoromethyl)cyclopropyl)phenyl) imidazolidine-2,4-dione |
| 182 | 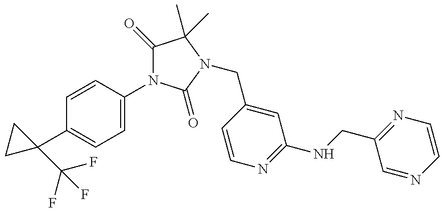 [SMILES: CC1(C)C(=O)N(c2ccc(C3(C(F)(F)F)CC3)cc2)C(=O)N1Cc1ccnc(NCc2cnccn2)c1] | 5,5-dimethyl-1-((2-((pyrazin-2- ylmethyl)amino)pyridin-4-yl)methyl)-3-(4- (1- (trifluoromethyl)cyclopropyl)phenyl) imidazolidine-2,4-dione |
| 183 | 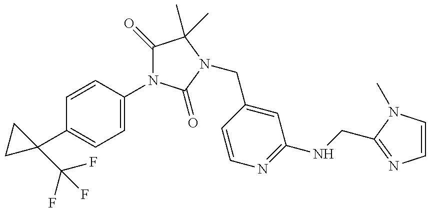 [SMILES: Cn1ccnc1CNc1cc(CN2C(=O)N(c3ccc(C4(C(F)(F)F)CC4)cc3)C(=O)C2(C)C)ccn1] | 5,5-dimethyl-1-((2-(((1-methyl-1H-imidazol- 2-yl)methyl)amino)pyridin-4-yl)methyl)-3- (4-(1- (trifluoromethyl)cyclopropyl)phenyl) imidazolidine-2,4-dione |
| 184 | 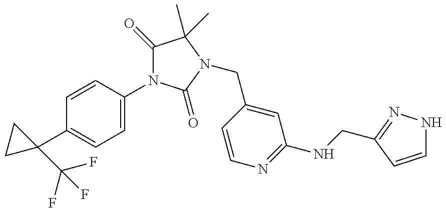 [SMILES: CC1(C)C(=O)N(c2ccc(C3(C(F)(F)F)CC3)cc2)C(=O)N1Cc1ccnc(NCc2cc[nH]n2)c1] | 1-((2-(((1H-pyrazol-3- yl)methyl)amino)pyridin-4-yl)methyl)-5,5- dimethyl-3-(4-(1- (trifluoromethyl)cyclopropyl)phenyl) imidazolidine-2,4-dione |

In some embodiments, the compound is a pharmaceutically acceptable salt of a compound described in Table 2.

TABLE 3

| Cmpd | | |
|---|---|---|
| # | Structure | Name |
| 185 | 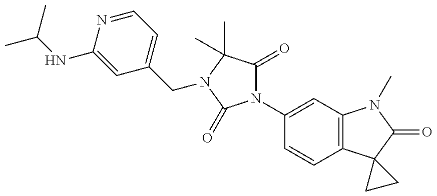 [SMILES: CC(C)Nc1cc(CN2C(=O)N(c3ccc4c(c3)N(C)C(=O)C43CC3)C(=O)C2(C)C)ccn1] | 1-((2-(isopropylamino)pyridin-4- yl)methyl)-5,5-dimethyl-3-(1′- methyl-2′-oxospiro[cyclopropane- 1,3′-indolin]-6′-yl)imidazolidine-2,4- dione |
| 186 | 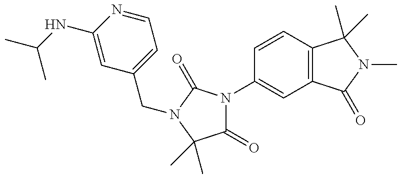 [SMILES: CC(C)Nc1cc(CN2C(=O)N(c3ccc4c(c3)C(=O)N(C)C4(C)C)C(=O)C2(C)C)ccn1] | 1-((2-(isopropylamino)pyridin-4- yl)methyl)-5,5-dimethyl-3-(1,1,2- trimethyl-3-oxoisoindolin-5- yl)imidazolidine-2,4-dione |
| 187 | 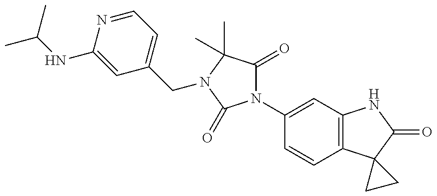 [SMILES: CC(C)Nc1cc(CN2C(=O)N(c3ccc4c(c3)NC(=O)C43CC3)C(=O)C2(C)C)ccn1] | 1-((2-(isopropylamino)pyridin-4- yl)methyl)-5,5-dimethyl-3-(2′- oxospiro[cyclopropane-1,3′- indolin]-6′-yl)imidazolidine-2,4-dione |
| 188 | 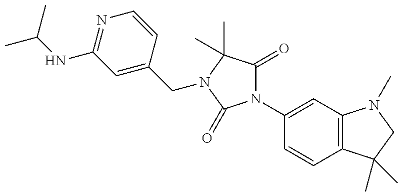 [SMILES: CC(C)Nc1cc(CN2C(=O)N(c3ccc4c(c3)N(C)CC4(C)C)C(=O)C2(C)C)ccn1] | 1-((2-(isopropylamino)pyridin-4- yl)methyl)-5,5-dimethyl-3-(1,3,3- trimethylindolin-6-yl)imidazolidine-2,4- dione |
| 189 | 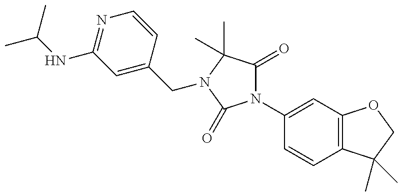 [SMILES: CC(C)Nc1cc(CN2C(=O)N(c3ccc4c(c3)OCC4(C)C)C(=O)C2(C)C)ccn1] | 3-(3,3-dimethyl-2,3- dihydrobenzofuran-6-yl)-1-((2- (isopropylamino)pyridin-4- yl)methyl)-5,5- dimethylimidazolidine-2,4-dione |
| 190 | 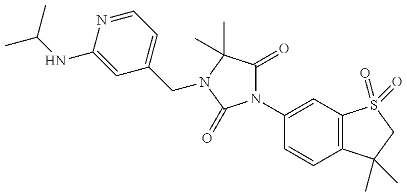 [SMILES: CC(C)Nc1cc(CN2C(=O)N(c3ccc4c(c3)S(=O)(=O)CC4(C)C)C(=O)C2(C)C)ccn1] | 3-(3,3-dimethyl-1,1-dioxido-2,3- dihydrobenzo[b]thiophen-6-yl)-1- ((2-(isopropylamino)pyridin-4- yl)methyl)-5,5- dimethylimidazolidine-2,4-dione |
| 191 | 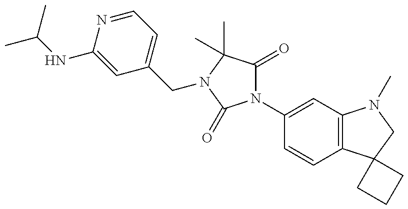 [SMILES: CC(C)Nc1cc(CN2C(=O)N(c3ccc4c(c3)N(C)CC43CCC3)C(=O)C2(C)C)ccn1] | 1-((2-(isopropylamino)pyridin-4- yl)methyl)-5,5-dimethyl-3-(1′- methylspiro[cyclobutane-1,3′- indolin]-6′-yl)imidazolidine-2,4- dione |
| 192 | 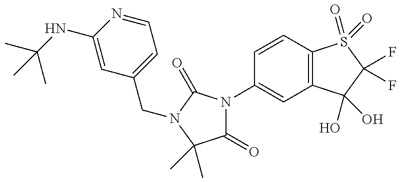 [SMILES: CC(C)(C)Nc1cc(CN2C(=O)N(c3ccc4c(c3)C(O)(O)C(F)(F)S4(=O)=O)C(=O)C2(C)C)ccn1] | 3-(2,2-difluoro-3,3-dihydroxy- 1,1-dioxido-2,3- dihydrobenzo[b]thiophen-5-yl)-1- ((2-(isopropylamino)pyridin-4- yl)methyl)-5,5- dimethylimidazolidine-2,4-dione |
| 193 | 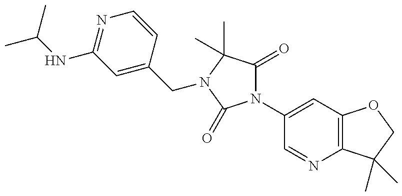 [SMILES: CC(C)Nc1cc(CN2C(=O)N(c3cnc4c(c3)OCC4(C)C)C(=O)C2(C)C)ccn1] | 3-(3,3-dimethyl-2,3- dihydrofuro[3,2-b]pyridin-6-yl)- 1-((2-(isopropylamino)pyridin-4- yl)methyl)-5,5- dimethylimidazolidine-2,4-dione |
| 194 | 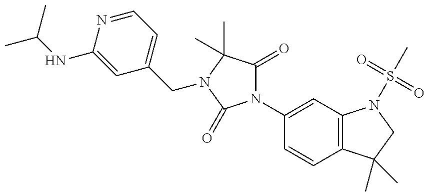 [SMILES: CC(C)Nc1cc(CN2C(=O)N(c3ccc4c(c3)N(S(C)(=O)=O)CC4(C)C)C(=O)C2(C)C)ccn1] | 3-(3,3-dimethyl-1- (methylsulfonyl)indolin-6-yl)-1- ((2-(isopropylamino)pyridin-4- yl)methyl)-5,5- dimethylimidazolidine-2,4-dione |
| 195 | 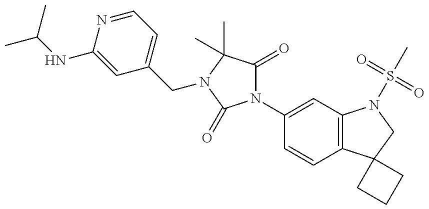 [SMILES: CC(C)Nc1cc(CN2C(=O)N(c3ccc4c(c3)N(S(C)(=O)=O)CC43CCC3)C(=O)C2(C)C)ccn1] | 1-((2-(isopropylamino)pyridin-4- yl)methyl)-5,5-dimethyl-3-(1′- (methylsulfonyl)spiro[cyclobutane- 1,3′-indolin]-6′- yl)imidazolidine-2,4-dione |
| 196 | 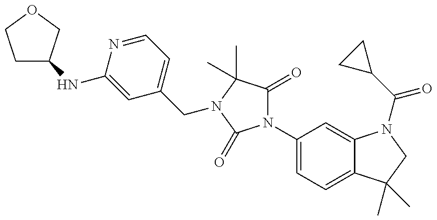 [SMILES: CC1(C)CN(C(=O)C2CC2)c2cc(N3C(=O)N(Cc4ccnc(N[C@H]5CCOC5)c4)C(C)(C)C3=O)ccc21] | (S)-3-(1-(cyclopropanecarbonyl)- 3,3-dimethylindolin-6-yl)-5,5- dimethyl-1-((2-((tetrahydrofuran- 3-yl)amino)pyridin-4- yl)methyl)imidazolidine-2,4- dione |
| 197 | 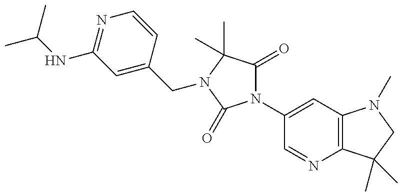 [SMILES: CC(C)Nc1cc(CN2C(=O)N(c3cnc4c(c3)N(C)CC4(C)C)C(=O)C2(C)C)ccn1] | 1-((2-(isopropylamino)pyridin-4- yl)methyl)-5,5-dimethyl-3-(1,3,3- trimethyl-2,3-dihydro-1H- pyrrolo[3,2-b]pyridin-6- yl)imidazolidine-2,4-dione |
| 198 | 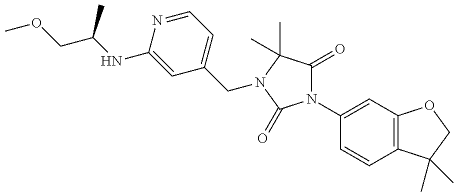 | (R)-3-(3,3-dimethyl-2,3- dihydrobenzofuran-6-yl)-1-((2- ((1-methoxypropan-2- yl)amino)pyridin-4-yl)methyl)- 5,5-dimethylimidazolidine-2,4- dione |
| 199 | 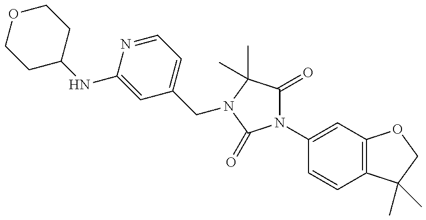 [SMILES: CC1(C)COc2cc(N3C(=O)N(Cc4ccnc(NC5CCOCC5)c4)C(C)(C)C3=O)ccc21] | 3-(3,3-dimethyl-2,3- dihydrobenzofuran-6-yl)-5,5- dimethyl-1-((2-((tetrahydro-2H- pyran-4-yl)amino)pyridin-4- yl)methyl)imidazolidine-2,4- dione |
| 200 | 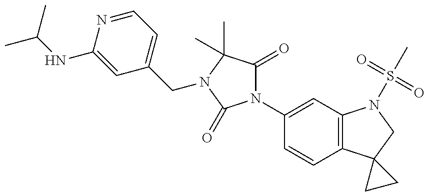 [SMILES: CC(C)Nc1cc(CN2C(=O)N(c3ccc4c(c3)N(S(C)(=O)=O)CC43CC3)C(=O)C2(C)C)ccn1] | 1-((2-(isopropylamino)pyridin-4- yl)methyl)-5,5-dimethyl-3-(1′- (methylsulfonyl)spiro[cyclopropane- 1,3′-indolin]-6′- yl)imidazolidine-2,4-dione |
| 201 | 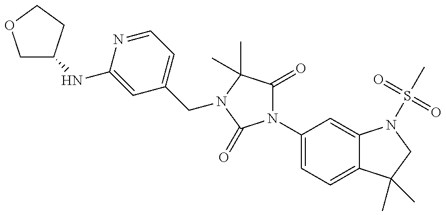 [SMILES: CC1(C)CN(S(C)(=O)=O)c2cc(N3C(=O)N(Cc4ccnc(N[C@H]5CCOC5)c4)C(C)(C)C3=O)ccc21] | (S)-3-(3,3-dimethyl-1- (methylsulfonyl)indolin-6-yl)-5,5- dimethyl-1-((2-((tetrahydrofuran- 3-yl)amino)pyridin-4- yl)methyl)imidazolidine-2,4- dione |
| 202 | 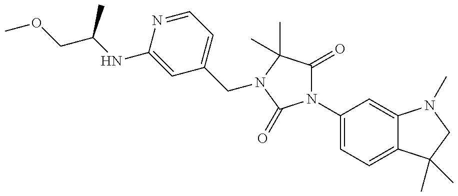 | (R)-1-((2-((1-methoxypropan-2- yl)amino)pyridin-4-yl)methyl)- 5,5-dimethyl-3-(1,3,3- trimethylindolin-6- yl)imidazolidine-2,4-dione |
| 203 | 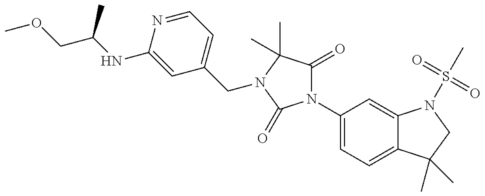 [SMILES: COC[C@@H](C)Nc1cc(CN2C(=O)N(c3ccc4c(c3)N(S(C)(=O)=O)CC4(C)C)C(=O)C2(C)C)ccn1] | (R)-3-(3,3-dimethyl-1- (methylsulfonyl)indolin-6-yl)-1- ((2-((1-methoxypropan-2- yl)amino)pyridin-4-yl)methyl)- 5,5-dimethylimidazolidine-2,4- dione |
| 204 | 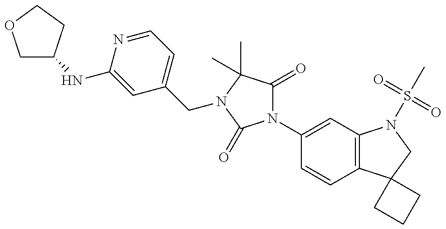 [SMILES: CC1(C)C(=O)N(c2ccc3c(c2)N(S(C)(=O)=O)CC32CCC2)C(=O)N1Cc1ccnc(N[C@H]2CCOC2)c1] | (S)-5,5-dimethyl-3-(1′- (methylsulfonyl)spiro[cyclobutane- 1,3′-indolin]-6′-yl)-1-((2- ((tetrahydrofuran-3- yl)amino)pyridin-4- yl)methyl)imidazolidine-2,4- dione |
| 205 | 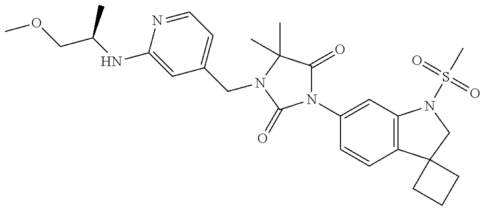 [SMILES: COC[C@@H](C)Nc1cc(CN2C(=O)N(c3ccc4c(c3)N(S(C)(=O)=O)CC43CCC3)C(=O)C2(C)C)ccn1] | (R)-1-((2-((1-methoxypropan-2- yl)amino)pyridin-4-yl)methyl)- 5,5-dimethyl-3-(1′- (methylsulfonyl)spiro[cyclobutane- 1,3′-indolin]-6′- yl)imidazolidine-2,4-dione |
| 206 | 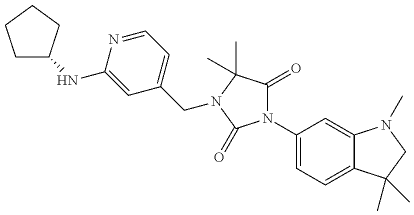 | (S)-5,5-dimethyl-1-((2- ((tetrahydrofuran-3- yl)amino)pyridin-4-yl)methyl)-3- (1,3,3-trimethylindolin-6- yl)imidazolidine-2,4-dione |
| 207 | 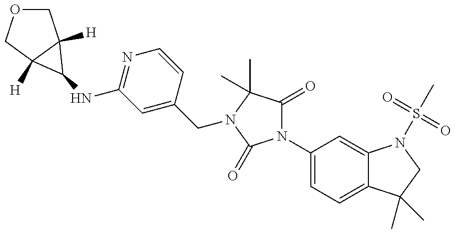 [SMILES: CC1(C)CN(S(C)(=O)=O)c2cc(N3C(=O)N(Cc4ccnc(N[C@H]5[C@@H]6COC[C@@H]65)c4)C(C)(C)C3=O)ccc21] | 1-((2-(((1R,5S,6r)-3- oxabicyclo[3.1.0]hexan-6- yl)amino)pyridin-4-yl)methyl)-3- (3,3-dimethyl-1- (methylsulfonyl)indolin-6-yl)-5,5- dimethylimidazolidine-2,4-dione |
| 208 | 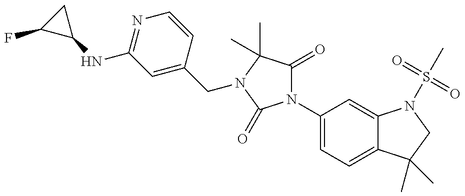 [SMILES: CC1(C)CN(S(C)(=O)=O)c2cc(N3C(=O)N(Cc4ccnc(N[C@@H]5C[C@@H]5F)c4)C(C)(C)C3=O)ccc21] | 3-(3,3-dimethyl-1- (methylsulfonyl)indolin-6-yl)-1- ((2-(((1R,2S)-2- fluorocyclopropyl)amino)pyridin- 4-yl)methyl)-5,5- dimethylimidazolidine-2,4-dione |
| 209 | 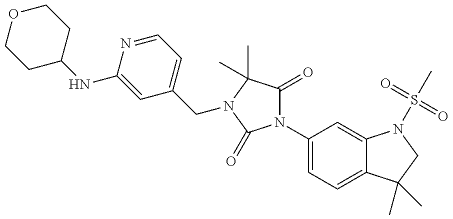 [SMILES: CC1(C)CN(S(C)(=O)=O)c2cc(N3C(=O)N(Cc4ccnc(NC5CCOCC5)c4)C(C)(C)C3=O)ccc21] | 3-(3,3-dimethyl-1- (methylsulfonyl)indolin-6-yl)-5,5- dimethyl-1-((2-((tetrahydro-2H- pyran-4-yl)amino)pyridin-4- yl)methyl)imidazolidine-2,4- dione |
| 210 | 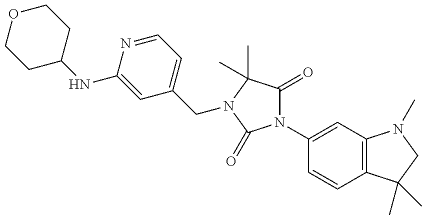 [SMILES: CN1CC(C)(C)c2ccc(N3C(=O)N(Cc4ccnc(NC5CCOCC5)c4)C(C)(C)C3=O)cc21] | 5,5-dimethyl-1-((2-((tetrahydro- 2H-pyran-4-yl)amino)pyridin-4- yl)methyl)-3-(1,3,3- trimethylindolin-6- yl)imidazolidine-2,4-dione |
| 211 | 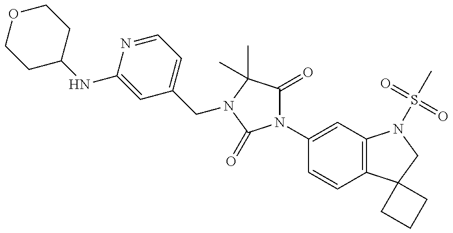 [SMILES: CC1(C)C(=O)N(c2ccc3c(c2)N(S(C)(=O)=O)CC32CCC2)C(=O)N1Cc1ccnc(NC2CCOCC2)c1] | 5,5-dimethyl-3-(1′- (methylsulfonyl)spiro[cyclobutane- 1,3′-indolin]-6′-yl)-1-((2- ((tetrahydro-2H-pyran-4- yl)amino)pyridin-4- yl)methyl)imidazolidine-2,4- dione |
| 212 | 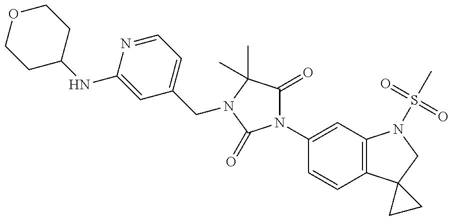 [SMILES: CC1(C)C(=O)N(c2ccc3c(c2)N(S(C)(=O)=O)CC32CC2)C(=O)N1Cc1ccnc(NC2CCOCC2)c1] | 5,5-dimethyl-3-(1′- (methylsulfonyl)spiro[cyclopropane- 1,3′-indolin]-6′-yl)-1-((2- ((tetrahydro-2H-pyran-4- yl)amino)pyridin-4- yl)methyl)imidazolidine-2,4- dione |
| 213 | 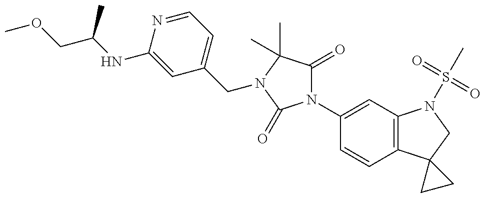 [SMILES: COC[C@@H](C)Nc1cc(CN2C(=O)N(c3ccc4c(c3)N(S(C)(=O)=O)CC43CC3)C(=O)C2(C)C)ccn1] | (R)-1-((2-((1-methoxypropan-2- yl)amino)pyridin-4-yl)methyl)- 5,5-dimethyl-3-(1′- (methylsulfonyl)spiro[cyclopropane- 1,3′-indolin]-6′- yl)imidazolidine-2,4-dione |
| 214 | 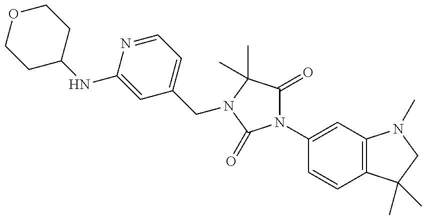 [SMILES: CN1CC(C)(C)c2ccc(N3C(=O)N(Cc4ccnc(NC5CCOCC5)c4)C(C)(C)C3=O)cc21] | 5,5-dimethyl-3-(1′- methylspiro[cyclobutane-1,3′- indolin]-6′-yl)-1-((2-((tetrahydro- 2H-pyran-4-yl)amino)pyridin-4- yl)methyl)imidazolidine-2,4- dione |
| 215 | 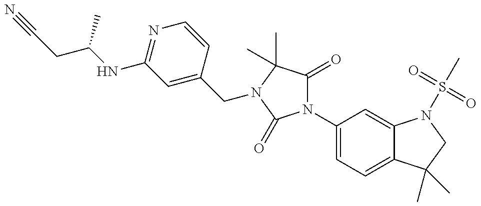 [SMILES: C[C@@H](CC#N)Nc1cc(CN2C(=O)N(c3ccc4c(c3)N(S(C)(=O)=O)CC4(C)C)C(=O)C2(C)C)ccn1] | (S)-3-((4-((3-(3,3-dimethyl-1- (methylsulfonyl)indolin-6-yl)-5,5- dimethyl-2,4-dioxoimidazolidin- 1-yl)methyl)pyridin-2- yl)amino)butanenitrile |
| 216 | 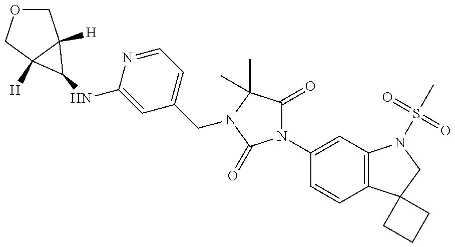 [SMILES: CC1(C)C(=O)N(c2ccc3c(c2)N(S(C)(=O)=O)CC32CCC2)C(=O)N1Cc1ccnc(N[C@H]2[C@@H]3COC[C@@H]32)c1] | 1-((2-(((1R,5S,6r)-3- oxabicyclo[3.1.0]hexan-6- yl)amino)pyridin-4-yl)methyl)- 5,5-dimethyl-3-(1′- (methylsulfonyl)spiro[cyclobutane- 1,3′-indolin]-6′- yl)imidazolidine-2,4-dione |
| 217 | 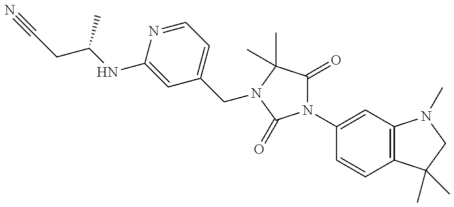 [SMILES: C[C@@H](CC#N)Nc1cc(CN2C(=O)N(c3ccc4c(c3)N(C)CC4(C)C)C(=O)C2(C)C)ccn1] | (S)-3-((4-((5,5-dimethyl-2,4- dioxo-3-(1,3,3-trimethylindolin-6- yl)imidazolidin-1- yl)methyl)pyridin-2- yl)amino)butanenitrile |
| 218 | 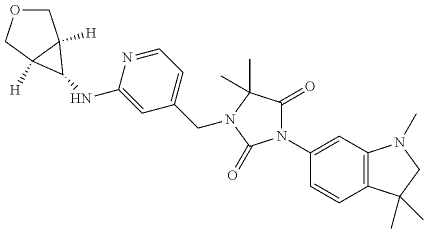 [SMILES: CN1CC(C)(C)c2ccc(N3C(=O)N(Cc4ccnc(N[C@H]5[C@@H]6COC[C@@H]65)c4)C(C)(C)C3=O)cc21] | 1-((2-(((1R,5S,6r)-3- oxabicyclo[3.1.0]hexan-6- yl)amino)pyridin-4-yl)methyl)- 5,5-dimethyl-3-(1,3,3- trimethylindolin-6- yl)imidazolidine-2,4-dione |
| 219 | 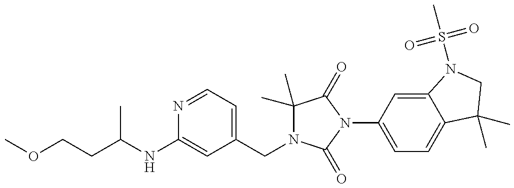 | 3-(3,3-dimethyl-1- (methylsulfonyl)indolin-6-yl)-1- ((2-((4-methoxybutan-2- yl)amino)pyridin-4-yl)methyl)- 5,5-dimethylimidazolidine-2,4- dione |
| 220 | 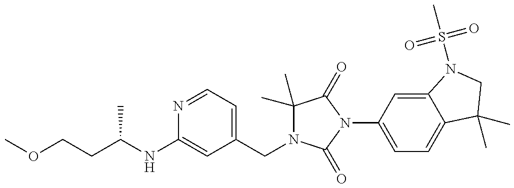 | (S)-3-(3,3-dimethyl-1- (methylsulfonyl)indolin-6-yl)-1- ((2-((1-ethoxypropan-2- yl)amino)pyridin-4-yl)methyl)- 5,5-dimethylimidazolidine-2,4- dione |
| 221 | 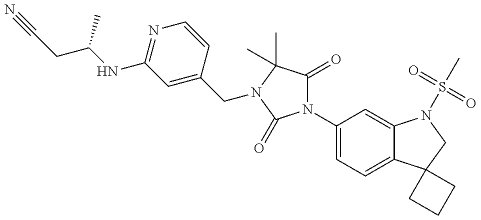 [SMILES: C[C@@H](CC#N)Nc1cc(CN2C(=O)N(c3ccc4c(c3)N(S(C)(=O)=O)CC43CCC3)C(=O)C2(C)C)ccn1] | (S)-3-((4-((5,5-dimethyl-3-(1′- (methylsulfonyl)spiro[cyclobutane- 1,3′-indolin]-6′-yl)-2,4- dioxoimidazolidin-1- yl)methyl)pyridin-2- yl)amino)butanenitrile |
| 222 | 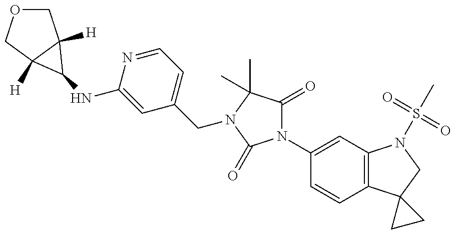 [SMILES: CC1(C)C(=O)N(c2ccc3c(c2)N(S(C)(=O)=O)CC32CC2)C(=O)N1Cc1ccnc(N[C@H]2[C@@H]3COC[C@@H]32)c1] | 1-((2-(((1R,5S,6r)-3- oxabicyclo[3.1.0]hexan-6- yl)amino)pyridin-4-yl)methyl)- 5,5-dimethyl-3-(1′- (methylsulfonyl)spiro[cyclopropane- 1,3′-indolin]-6′- yl)imidazolidine-2,4-dione |
| 223 | 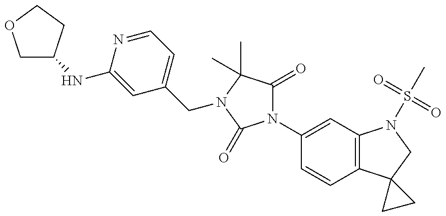 [SMILES: CC1(C)C(=O)N(c2ccc3c(c2)N(S(C)(=O)=O)CC32CC2)C(=O)N1Cc1ccnc(N[C@H]2CCOC2)c1] | (S)-5,5-dimethyl-3-(1′- (methylsulfonyl)spiro[cyclopropane- 1,3′-indolin]-6′-yl)-1-((2- ((tetrahydrofuran-3- yl)amino)pyridin-4- yl)methyl)imidazolidine-2,4- dione |
| 224 | 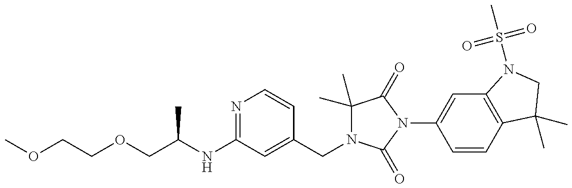 | (R)-3-(3,3-dimethyl-1- (methylsulfonyl)indolin-6-yl)-1- ((2-((1-(2-methoxyethoxy)propan- 2-yl)amino)pyridin-4-yl)methyl)- 5,5-dimethylimidazolidine-2,4- dione |
| 225 | 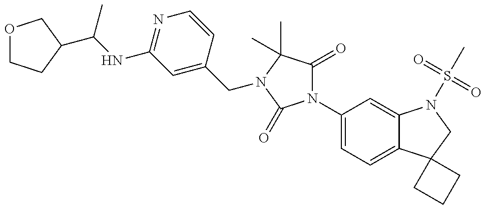 [SMILES: CC(Nc1cc(CN2C(=O)N(c3ccc4c(c3)N(S(C)(=O)=O)CC43CCC3)C(=O)C2(C)C)ccn1)C1CCOC1] | 5,5-dimethyl-3-(1′- (methylsulfonyl)spiro[cyclobutane- 1,3′-indolin]-6′-yl)-1-((2-((1- (tetrahydrofuran-3- yl)ethyl)amino)pyridin-4- yl)methyl)imidazolidine-2,4- dione |
| 226 | 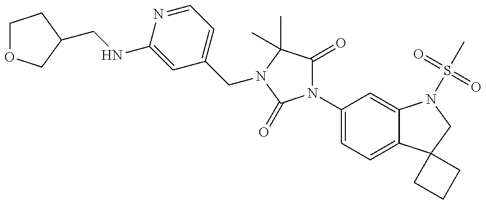 [SMILES: CC1(C)C(=O)N(c2ccc3c(c2)N(S(C)(=O)=O)CC32CCC2)C(=O)N1Cc1ccnc(NCC2CCOC2)c1] | 5,5-dimethyl-3-(1′- (methylsulfonyl)spiro[cyclobutane- 1,3′-indolin]-6′-yl)-1-((2- (((tetrahydrofuran-3- yl)methyl)amino)pyridin-4- yl)methyl)imidazolidine-2,4- dione |
| 227 | 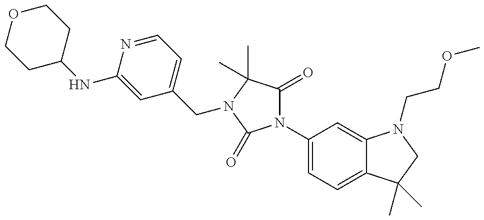 [SMILES: COCCN1CC(C)(C)c2ccc(N3C(=O)N(Cc4ccnc(NC5CCOCC5)c4)C(C)(C)C3=O)cc21] | 3-(1-(2-methoxyethyl)-3,3- dimethylindolin-6-yl)-5,5- dimethyl-1-((2-((tetrahydro-2H- pyran-4-yl)amino)pyridin-4- yl)methyl)imidazolidine-2,4- dione |
| 228 | 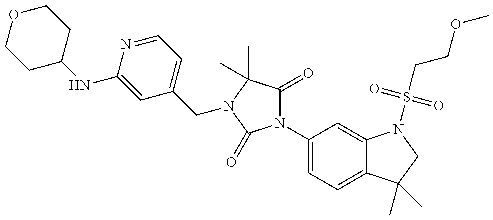 [SMILES: COCCS(=O)(=O)N1CC(C)(C)c2ccc(N3C(=O)N(Cc4ccnc(NC5CCOCC5)c4)C(C)(C)C3=O)cc21] | 3-(1-((2-methoxyethyl)sulfonyl)- 3,3-dimethylindolin-6-yl)-5,5- dimethyl-1-((2-((tetrahydro-2H- pyran-4-yl)amino)pyridin-4- yl)methyl)imidazolidine-2,4- dione |
| 229 | 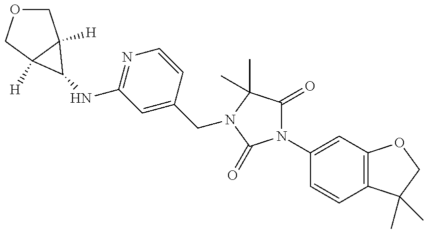 [SMILES: CC1(C)COc2cc(N3C(=O)N(Cc4ccnc(N[C@H]5[C@@H]6COC[C@@H]65)c4)C(C)(C)C3=O)ccc21] | 1-((2-(((1R,5S,6r)-3- oxabicyclo[3.1.0]hexan-6- yl)amino)pyridin-4-yl)methyl)-3- (3,3-dimethyl-2,3- dihydrobenzofuran-6-yl)-5,5- dimethylimidazolidine-2,4-dione |
| 230 | 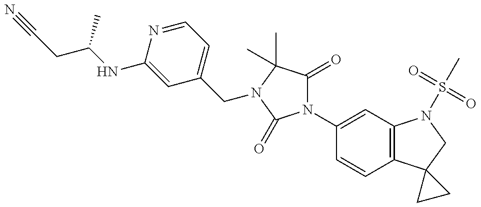 [SMILES: C[C@@H](CC#N)Nc1cc(CN2C(=O)N(c3ccc4c(c3)N(S(C)(=O)=O)CC43CC3)C(=O)C2(C)C)ccn1] | (S)-3-((4-((5,5-dimethyl-3-(1′- (methylsulfonyl)spiro[cyclopropane- 1,3′-indolin]-6′-yl)-2,4- dioxoimidazolidin-1- yl)methyl)pyridin-2- yl)amino)butanenitrile |
| 231 | 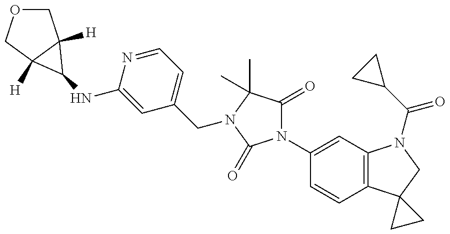 [SMILES: CC1(C)C(=O)N(c2ccc3c(c2)N(C(=O)C2CC2)CC32CC2)C(=O)N1Cc1ccnc(N[C@H]2[C@@H]3COC[C@@H]32)c1] | 1-((2-(((1R,5S,6r)-3- oxabicyclo[3.1.0]hexan-6- yl)amino)pyridin-4-yl)methyl)-3- (1′- (cyclopropanecarbonyl)spiro [cyclopropane-1,3′-indolin]-6′-yl)-5,5- dimethylimidazolidine-2,4-dione |
| 232 | 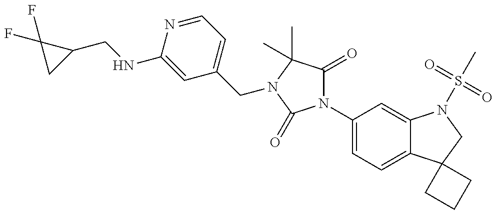 [SMILES: CC1(C)C(=O)N(c2ccc3c(c2)N(S(C)(=O)=O)CC32CCC2)C(=O)N1Cc1ccnc(NCC2CC2(F)F)c1] | 1-((2-(((2,2- difluorocyclopropyl)methyl)amino) pyridin-4-yl)methyl)-5,5- dimethyl-3-(1′- (methylsulfonyl)spiro[cyclobutane- 1,3′-indolin]-6′- yl)imidazolidine-2,4-dione |
| 233 | 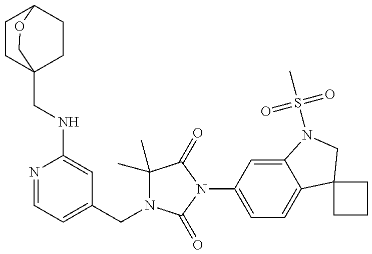 [SMILES: CC1(C)C(=O)N(c2ccc3c(c2)N(S(C)(=O)=O)CC32CCC2)C(=O)N1Cc1ccnc(NCC23CCC(CC2)OC3)c1] | 1-((2-(((2-oxabicyclo[2.2.2]octan- 4-yl)methyl)amino)pyridin-4- yl)methyl)-5,5-dimethyl-3-(1′- (methylsulfonyl)spiro[cyclobutane- 1,3′-indolin]-6′- yl)imidazolidine-2,4-dione |
| 234 | 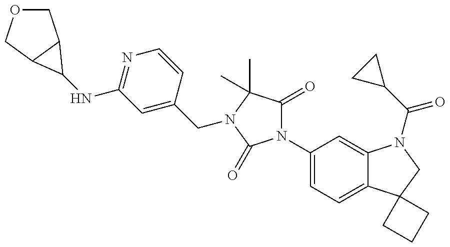 [SMILES: CC1(C)C(=O)N(c2ccc3c(c2)N(C(=O)C2CC2)CC32CCC2)C(=O)N1Cc1ccnc(NC2C3COCC32)c1] | 1-((2-((3-oxabicyclo[3.1.0]hexan- 6-yl)amino)pyridin-4-yl)methyl)- 3-(1′- (cyclopropanecarbonyl)spiro [cyclobutane-1,3′-indolin]-6′-yl)-5,5- dimethylimidazolidine-2,4-dione |
| 235 | 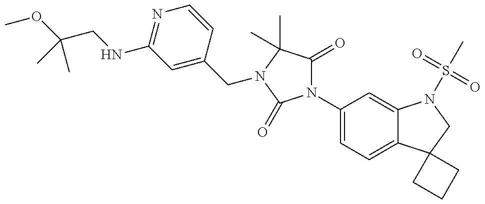 [SMILES: COC(C)(C)CNc1cc(CN2C(=O)N(c3ccc4c(c3)N(S(C)(=O)=O)CC43CCC3)C(=O)C2(C)C)ccn1] | 1-((2-((2-methoxy-2- methylpropyl)amino)pyridin-4- yl)methyl)-5,5-dimethyl-3-(1′- (methylsulfonyl)spiro[cyclobutane- 1,3′-indolin]-6′- yl)imidazolidine-2,4-dione |
| 236 | 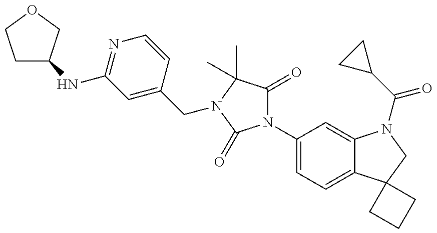 [SMILES: CC1(C)C(=O)N(c2ccc3c(c2)N(C(=O)C2CC2)CC32CCC2)C(=O)N1Cc1ccnc(N[C@H]2CCOC2)c1] | (S)-3-(1′- (cyclopropanecarbonyl)spiro [cyclobutane-1,3′-indolin]-6′-yl)-5,5- dimethyl-1-((2-((tetrahydrofuran- 3-yl)amino)pyridin-4- yl)methyl)imidazolidine-2,4- dione |
| 237 | 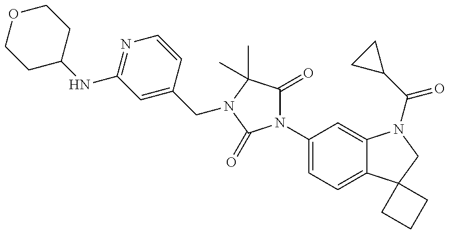 [SMILES: CC1(C)C(=O)N(c2ccc3c(c2)N(C(=O)C2CC2)CC32CCC2)C(=O)N1Cc1ccnc(NC2CCOCC2)c1] | 3-(1′- (cyclopropanecarbonyl)spiro [cyclobutane-1,3′-indolin]-6′-yl)-5,5- dimethyl-1-((2-((tetrahydro-2H- pyran-4-yl)amino)pyridin-4- yl)methyl)imidazolidine-2,4- dione |
| 238 | 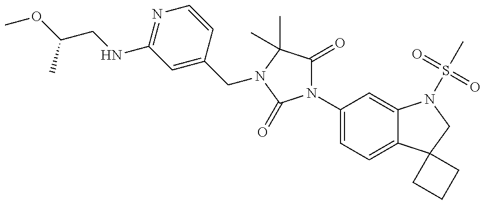 [SMILES: CO[C@@H](C)CNc1cc(CN2C(=O)N(c3ccc4c(c3)N(S(C)(=O)=O)CC43CCC3)C(=O)C2(C)C)ccn1] | (S)-1-((2-((2- methoxypropyl)amino)pyridin-4- yl)methyl)-5,5-dimethyl-3-(1′- (methylsulfonyl)spiro[cyclobutane- 1,3′-indolin]-6′- yl)imidazolidine-2,4-dione |
| 239 | 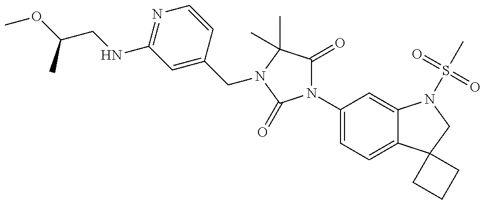 [SMILES: CO[C@H](C)CNc1cc(CN2C(=O)N(c3ccc4c(c3)N(S(C)(=O)=O)CC43CCC3)C(=O)C2(C)C)ccn1] | (R)-1-((2-((2- methoxypropyl)amino)pyridin-4- yl)methyl)-5,5-dimethyl-3-(1′- (methylsulfonyl)spiro[cyclobutane- 1,3′-indolin]-6′- yl)imidazolidine-2,4-dione |
| 240 | 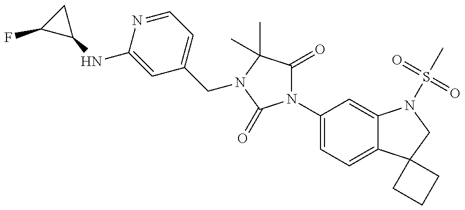 [SMILES: CC1(C)C(=O)N(c2ccc3c(c2)N(S(C)(=O)=O)CC32CCC2)C(=O)N1Cc1ccnc(N[C@@H]2C[C@@H]2F)c1] | 1-((2-(((1R,2S)-2- fluorocyclopropyl)amino)pyridin- 4-yl)methyl)-5,5-dimethyl-3-(1′- (methylsulfonyl)spiro[cyclobutane- 1,3′-indolin]-6′- yl)imidazolidine-2,4-dione |
| 241 | 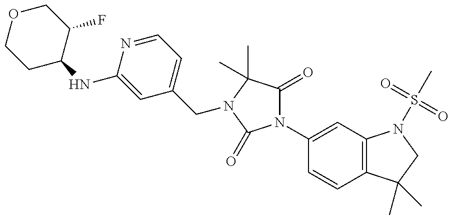 [SMILES: CC1(C)CN(S(C)(=O)=O)c2cc(N3C(=O)N(Cc4ccnc(N[C@H]5CCOC[C@@H]5F)c4)C(C)(C)C3=O)ccc21] | 3-(3,3-dimethyl-1- (methylsulfonyl)indolin-6-yl)-1- ((2-(((3R,4S)-3-fluorotetrahydro- 2H-pyran-4-yl)amino)pyridin-4- yl)methyl)-5,5- dimethylimidazolidine-2,4-dione |
| 242 | 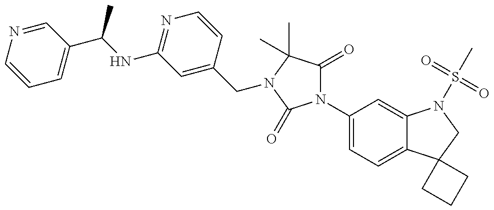 [SMILES: C[C@@H](Nc1cc(CN2C(=O)N(c3ccc4c(c3)N(S(C)(=O)=O)CC43CCC3)C(=O)C2(C)C)ccn1)c1cccnc1] | (R)-5,5-dimethyl-3-(1′- (methylsulfonyl)spiro[cyclobutane- 1,3′-indolin]-6′-yl)-1-((2-((1- (pyridin-3- yl)ethyl)amino)pyridin-4- yl)methyl)imidazolidine-2,4- dione |
| 243 | 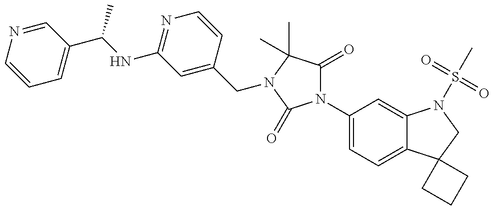 [SMILES: C[C@H](Nc1cc(CN2C(=O)N(c3ccc4c(c3)N(S(C)(=O)=O)CC43CCC3)C(=O)C2(C)C)ccn1)c1cccnc1] | (S)-5,5-dimethyl-3-(1′- (methylsulfonyl)spiro[cyclobutane- 1,3′-indolin]-6′-yl)-1-((2-((1- (pyridin-3- yl)ethyl)amino)pyridin-4- yl)methyl)imidazolidine-2,4- dione |
| 244 | 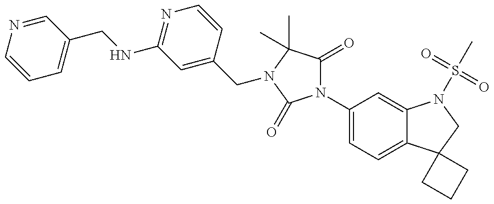 [SMILES: CC1(C)C(=O)N(c2ccc3c(c2)N(S(C)(=O)=O)CC32CCC2)C(=O)N1Cc1ccnc(NCc2cccnc2)c1] | 5,5-dimethyl-3-(1′- (methylsulfonyl)spiro[cyclobutane- 1,3′-indolin]-6′-yl)-1-((2- ((pyridin-3- ylmethyl)amino)pyridin-4- yl)methyl)imidazolidine-2,4- dione |
| 245 | 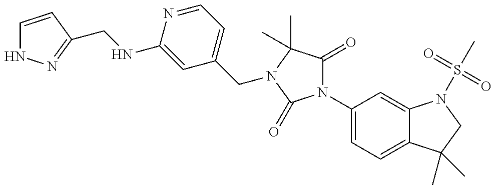 [SMILES: CC1(C)CN(S(C)(=O)=O)c2cc(N3C(=O)N(Cc4ccnc(NCc5cc[nH]n5)c4)C(C)(C)C3=O)ccc21] | 1-((2-(((1H-pyrazol-3- yl)methyl)amino)pyridin-4- yl)methyl)-3-(3,3-dimethyl-1- (methylsulfonyl)indolin-6-yl)-5,5- dimethylimidazolidine-2,4-dione |
| 246 | 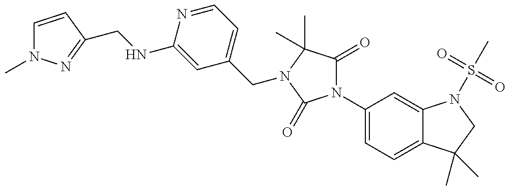 [SMILES: Cn1ccc(CNc2cc(CN3C(=O)N(c4ccc5c(c4)N(S(C)(=O)=O)CC5(C)C)C(=O)C3(C)C)ccn2)n1] | 3-(3,3-dimethyl-1- (methylsulfonyl)indolin-6-yl)-5,5- dimethyl-1-((2-(((1-methyl-1H- pyrazol-3- yl)methyl)amino)pyridin-4- yl)methyl)imidazolidine-2,4- dione |
| 247 | 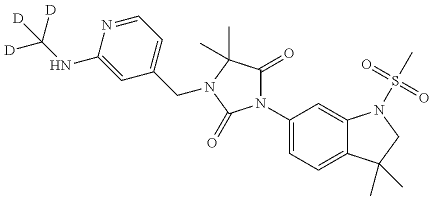 [SMILES: [2H]C([2H])([2H])Nc1cc(CN2C(=O)N(c3ccc4c(c3)N(S(C)(=O)=O)CC4(C)C)C(=O)C2(C)C)ccn1] | 3-(3,3-dimethyl-1- (methylsulfonyl)indolin-6-yl)-5,5- dimethyl-1-((2-((methyl- d3)amino)pyridin-4- yl)methyl)imidazolidine-2,4- dione |
| 248 | 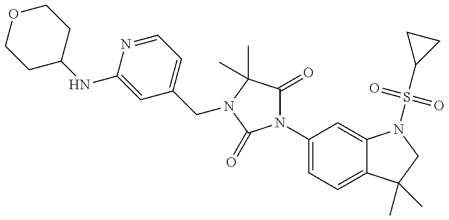 [SMILES: CC1(C)CN(S(=O)(=O)C2CC2)c2cc(N3C(=O)N(Cc4ccnc(NC5CCOCC5)c4)C(C)(C)C3=O)ccc21] | 3-(1-(cyclopropylsulfonyl)-3,3- dimethylindolin-6-yl)-5,5- dimethyl-1-((2-((tetrahydro-2H- pyran-4-yl)amino)pyridin-4- yl)methyl)imidazolidine-2,4- dione |
| 249 | 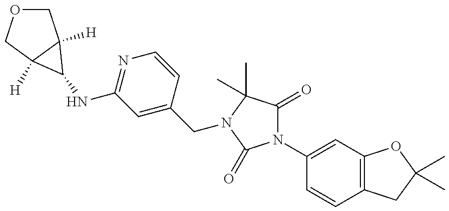 [SMILES: CC1(C)Cc2ccc(N3C(=O)N(Cc4ccnc(N[C@H]5[C@@H]6COC[C@@H]65)c4)C(C)(C)C3=O)cc2O1] | 1-((2-(((1R,5S,6r)-3- oxabicyclo[3.1.0]hexan-6- yl)amino)pyridin-4-yl)methyl)-3- (2,2-dimethyl-2,3- dihydrobenzofuran-6-yl)-5,5- dimethylimidazolidine-2,4-dione |
| 250 | 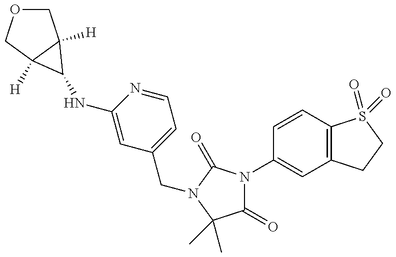 [SMILES: CC1(C)C(=O)N(c2ccc3c(c2)CCS3(=O)=O)C(=O)N1Cc1ccnc(N[C@H]2[C@@H]3COC[C@@H]32)c1] | 1-((2-(((1R,5S,6r)-3- oxabicyclo[3.1.0]hexan-6- yl)amino)pyridin-4-yl)methyl)-3- (1,1-dioxido-2,3- dihydrobenzo[b]thiophen-5-yl)- 5,5-dimethylimidazolidine-2,4- dione |
| 251 | 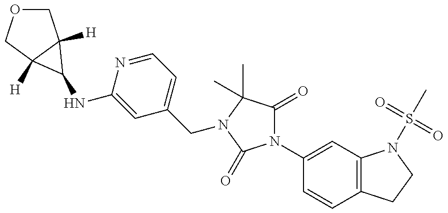 [SMILES: CC1(C)C(=O)N(c2ccc3c(c2)N(S(C)(=O)=O)CC3)C(=O)N1Cc1ccnc(N[C@H]2[C@@H]3COC[C@@H]32)c1] | 1-((2-(((1R,5S,6r)-3- oxabicyclo[3.1.0]hexan-6- yl)amino)pyridin-4-yl)methyl)- 5,5-dimethyl-3-(1- (methylsulfonyl)indolin-6- yl)imidazolidine-2,4-dione |
| 252 | 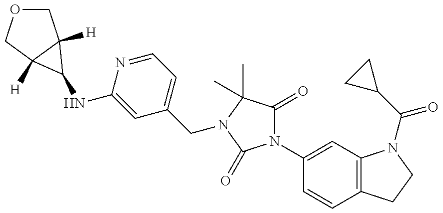 [SMILES: CC1(C)C(=O)N(c2ccc3c(c2)N(C(=O)C2CC2)CC3)C(=O)N1Cc1ccnc(N[C@H]2[C@@H]3COC[C@@H]32)c1] | 1-((2-(((1R,5S,6r)-3- oxabicyclo[3.1.0]hexan-6- yl)amino)pyridin-4-yl)methyl)-3- (1- (cyclopropanecarbonyl)indolin-6- yl)-5,5-dimethylimidazolidine- 2,4-dione |
| 253 | 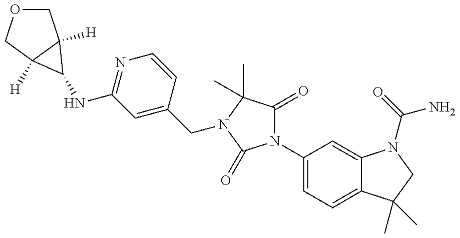 [SMILES: CC1(C)CN(C(N)=O)c2cc(N3C(=O)N(Cc4ccnc(N[C@H]5[C@@H]6COC[C@@H]65)c4)C(C)(C)C3=O)ccc21] | 6-(3-((2-(((1R,5S,6r)-3- oxabicyclo[3.1.0]hexan-6- yl)amino)pyridin-4-yl)methyl)- 4,4-dimethyl-2,5- dioxoimidazolidin-1-yl)-3,3- dimethylindoline-1-carboxamide |

In some embodiments, the compound is a pharmaceutically acceptable salt of a compound described in Table 3.

TABLE 4

| Cmpd # | Structure | Name |
|---|---|---|
| 254 | 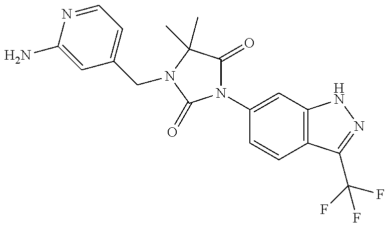 [SMILES: CC1(C)C(=O)N(c2ccc3c(C(F)(F)F)n[nH]c3c2)C(=O)N1Cc1ccnc(N)c1] | 1-((2-aminopyridin-4-yl)methyl)-5,5- dimethyl-3-(3-(trifluoromethyl)-1H- indazol-6-yl)imidazolidine-2,4-dione |
| 255 | 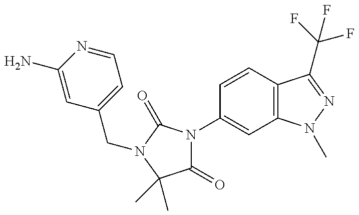 [SMILES: Cn1nc(C(F)(F)F)c2ccc(N3C(=O)N(Cc4ccnc(N)c4)C(C)(C)C3=O)cc21] | 1-((2-aminopyridin-4-yl)methyl)-5,5- dimethyl-3-(1-methyl-3-(trifluoromethyl)- 1H-indazol-6-yl)imidazolidine-2,4-dione |
| 256 | 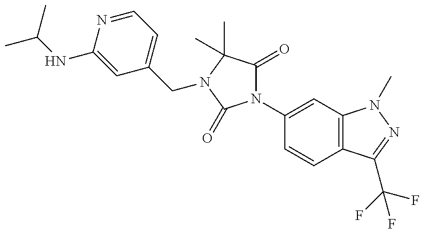 [SMILES: CC(C)Nc1cc(CN2C(=O)N(c3ccc4c(C(F)(F)F)nn(C)c4c3)C(=O)C2(C)C)ccn1] | 1-((2-(isopropylamino)pyridin-4- yl)methyl)-5,5-dimethyl-3-(1-methyl-3- (trifluoromethyl)-1H-indazol-6- yl)imidazolidine-2,4-dione |
| 257 | 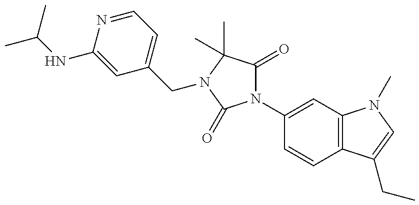 [SMILES: CCc1cn(C)c2cc(N3C(=O)N(Cc4ccnc(NC(C)C)c4)C(C)(C)C3=O)ccc12] | 3-(3-ethyl-1-methyl-1H-indol-6-yl)-1-((2- (isopropylamino)pyridin-4-yl)methyl)-5,5- dimethylimidazolidine-2,4-dione |
| 258 | 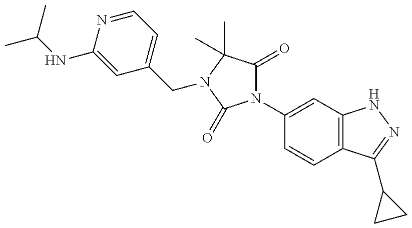 [SMILES: CC(C)Nc1cc(CN2C(=O)N(c3ccc4c(C5CC5)n[nH]c4c3)C(=O)C2(C)C)ccn1] | 3-(3-cyclopropyl-1H-indazol-6-yl)-1-((2- (isopropylamino)pyridin-4-yl)methyl)-5,5- dimethylimidazolidine-2,4-dione |

In some embodiments, the compound is a pharmaceutically acceptable salt of a compound described in Table 4.

TABLE 5

| Cmpd # | Structure | Name |
|---|---|---|
| 259 | 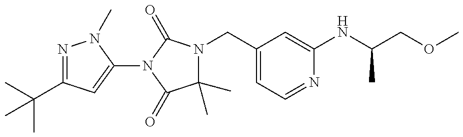 [SMILES: COC[C@@H](C)Nc1cc(CN2C(=O)N(c3cc(C(C)(C)C)nn3C)C(=O)C2(C)C)ccn1] | (R)-3-(3-(tert-butyl)-1-methyl-1H- pyrazol-5-yl)-1-((2-((1- methoxypropan-2-yl)amino)pyridin-4- yl)methyl)-5,5-dimethylimidazolidine- 2,4-dione |
| 260 | 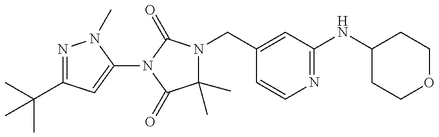 [SMILES: Cn1nc(C(C)(C)C)cc1N1C(=O)N(Cc2ccnc(NC3CCOCC3)c2)C(C)(C)C1=O] | 3-(3-(tert-butyl)-1-methyl-1H-pyrazol- 5-yl)-5,5-dimethyl-1-((2-((tetrahydro- 2H-pyran-4-yl)amino)pyridin-4- yl)methyl)imidazolidine-2,4-dione |
| 261 | 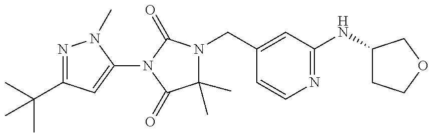 [SMILES: Cn1nc(C(C)(C)C)cc1N1C(=O)N(Cc2ccnc(N[C@H]3CCOC3)c2)C(C)(C)C1=O] | (S)-3-(3-(tert-butyl)-1-methyl-1H- pyrazol-5-yl)-5,5-dimethyl-1-((2- ((tetrahydrofuran-3-yl)amino)pyridin- 4-yl)methyl)imidazolidine-2,4-dione |
| 262 | 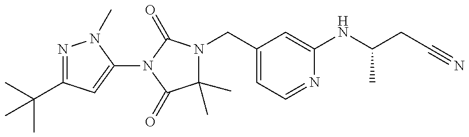 [SMILES: C[C@@H](CC#N)Nc1cc(CN2C(=O)N(c3cc(C(C)(C)C)nn3C)C(=O)C2(C)C)ccn1] | (S)-3-((4-((3-(3-(tert-butyl)-1-methyl- 1H-pyrazol-5-yl)-5,5-dimethyl-2,4- dioxoimidazolidin-1- yl)methyl)pyridin-2- yl)amino)butanenitrile |

In some embodiments, the compound is a pharmaceutically acceptable salt of a compound described in Table 5.

TABLE 6

| Cmpd # | Structure | Name |
|---|---|---|
| 263 | 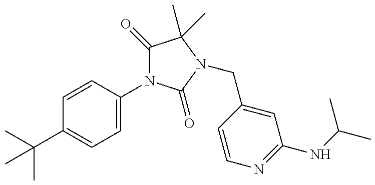 [SMILES: CC(C)Nc1cc(CN2C(=O)N(c3ccc(C(C)(C)C)cc3)C(=O)C2(C)C)ccn1] | 3-(4-(tert-butyl)phenyl)-1-((2- (isopropylamino)pyridin-4- yl)methyl)-5,5- dimethylimidazolidine-2,4- dione |
| 264 | 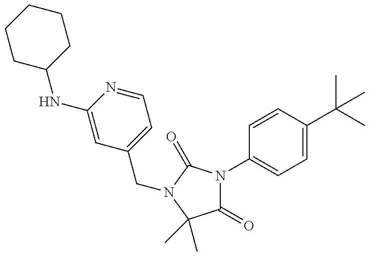 [SMILES: CC(C)(C)c1ccc(N2C(=O)N(Cc3ccnc(NC4CCCCC4)c3)C(C)(C)C2=O)cc1] | 3-(4-(tert-butyl)phenyl)-1-((2- (cyclohexylamino)pyridin-4- yl)methyl)-5,5- dimethylimidazolidine-2,4- dione |
| 265 | 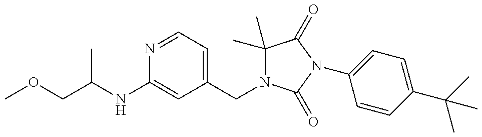 [SMILES: COCC(C)Nc1cc(CN2C(=O)N(c3ccc(C(C)(C)C)cc3)C(=O)C2(C)C)ccn1] | 3-(4-(tert-butyl)phenyl)-1-((2- ((1-methoxypropan-2- yl)amino)pyridin-4-yl)methyl)- 5,5-dimethylimidazolidine-2,4- dione |
| 266 | 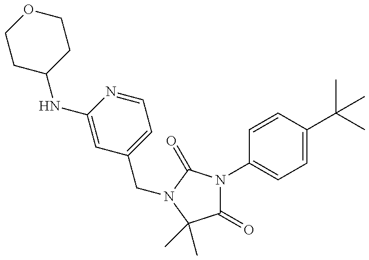 [SMILES: CC(C)(C)c1ccc(N2C(=O)N(Cc3ccnc(NC4CCOCC4)c3)C(C)(C)C2=O)cc1] | 3-(4-(tert-butyl)phenyl)-5,5- dimethyl-1-((2-((tetrahydro- 2H-pyran-4-yl)amino)pyridin- 4-yl)methyl)imidazolidine-2,4- dione |
| 267 | 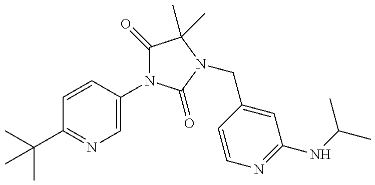 [SMILES: CC(C)Nc1cc(CN2C(=O)N(c3ccc(C(C)(C)C)nc3)C(=O)C2(C)C)ccn1] | 3-(6-(tert-butyl)pyridin-3-yl)- 1-((2-(isopropylamino)pyridin- 4-yl)methyl)-5,5- dimethylimidazolidine-2,4- dione |
| 268 | 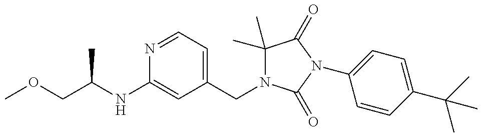 [SMILES: COC[C@@H](C)Nc1cc(CN2C(=O)N(c3ccc(C(C)(C)C)cc3)C(=O)C2(C)C)ccn1] | (R)-3-(4-(tert-butyl)phenyl)-1- ((2-((1-methoxypropan-2- yl)amino)pyridin-4-yl)methyl)- 5,5-dimethylimidazolidine-2,4- dione |
| 269 | 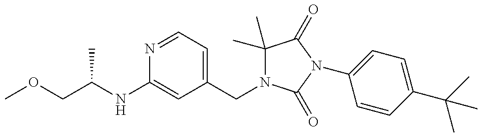 [SMILES: COC[C@H](C)Nc1cc(CN2C(=O)N(c3ccc(C(C)(C)C)cc3)C(=O)C2(C)C)ccn1] | (S)-3-(4-(tert-butyl)phenyl)-1- ((2-((1-methoxypropan-2- yl)amino)pyridin-4-yl)methyl)- 5,5-dimethylimidazolidine-2,4- dione |
| 270 | 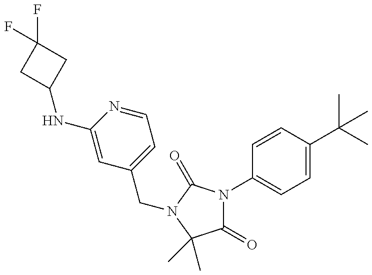 [SMILES: CC(C)(C)c1ccc(N2C(=O)N(Cc3ccnc(NC4CC(F)(F)C4)c3)C(C)(C)C2=O)cc1] | 3-(4-(tert-butyl)phenyl)-1-((2- ((3,3- difluorocyclobutyl)amino) pyridin-4-yl)methyl)-5,5- dimethylimidazolidine-2,4- dione |
| 271 | 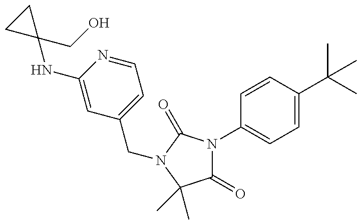 [SMILES: CC(C)(C)c1ccc(N2C(=O)N(Cc3ccnc(NC4(CO)CC4)c3)C(C)(C)C2=O)cc1] | 3-(4-(tert-butyl)phenyl)-1-((2- ((1- (hydroxymethyl)cyclopropyl) amino)pyridin-4-yl)methyl)- 5,5-dimethylimidazolidine-2,4- dione |
| 272 | 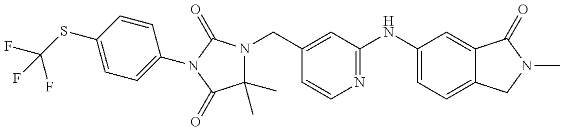 [SMILES: CN1Cc2ccc(Nc3cc(CN4C(=O)N(c5ccc(SC(F)(F)F)cc5)C(=O)C4(C)C)ccn3)cc2C1=O] | 5,5-dimethyl-1-((2-((2-methyl- 3-oxoisoindolin-5- yl)amino)pyridin-4-yl)methyl)- 3-(4- ((trifluoromethyl)thio)phenyl) amidazolidine-2,4-dione |
| 273 | 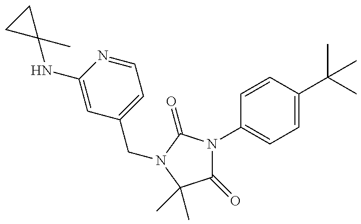 [SMILES: CC1(Nc2cc(CN3C(=O)N(c4ccc(C(C)(C)C)cc4)C(=O)C3(C)C)ccn2)CC1] | 3-(4-(tert-butyl)phenyl)-5,5- dimethyl-1-((2-((1- methylcyclopropyl)amino) pyridin-4-yl)methyl) imidazolidine-2,4-dione |
| 274 | 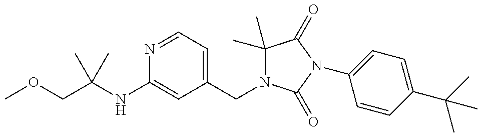 [SMILES: COCC(C)(C)Nc1cc(CN2C(=O)N(c3ccc(C(C)(C)C)cc3)C(=O)C2(C)C)ccn1] | 3-(4-(tert-butyl)phenyl)-1-((2- ((1-methoxy-2-methylpropan- 2-yl)amino)pyridin-4- yl)methyl)-5,5- dimethylimidazolidine-2,4- dione |
| 275 | 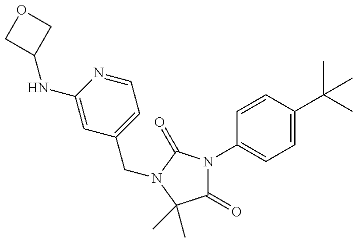 [SMILES: CC(C)(C)c1ccc(N2C(=O)N(Cc3ccnc(NC4COC4)c3)C(C)(C)C2=O)cc1] | 3-(4-(tert-butyl)phenyl)-5,5- dimethyl-1-((2-(oxetan-3- ylamino)pyridin-4- yl)methyl)imidazolidine-2,4- dione |
| 276 | 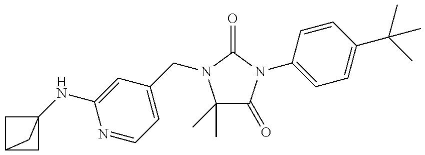 [SMILES: CC(C)(C)c1ccc(N2C(=O)N(Cc3ccnc(NC45CC(C4)C5)c3)C(C)(C)C2=O)cc1] | 1-((2-(bicyclo[1.1.1]pentan-1- ylamino)pyridin-4-yl)methyl)- 3-(4-(tert-butyl)phenyl)-5,5- dimethylimidazolidine-2,4- dione |
| 277 | 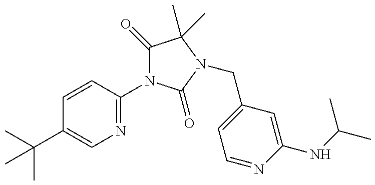 [SMILES: CC(C)Nc1cc(CN2C(=O)N(c3ccc(C(C)(C)C)cn3)C(=O)C2(C)C)ccn1] | 3-(5-(tert-butyl)pyridin-2-yl)- 1-((2-(isopropylamino)pyridin- 4-yl)methyl)-5,5- dimethylimidazolidine-2,4- dione |
| 278 | 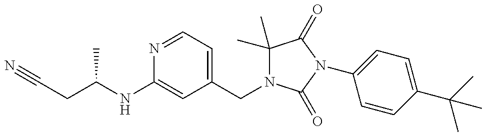 [SMILES: C[C@@H](CC#N)Nc1cc(CN2C(=O)N(c3ccc(C(C)(C)C)cc3)C(=O)C2(C)C)ccn1] | (S)-3-((4-((3-(4-(tert- butyl)phenyl)-5,5-dimethyl- 2,4-dioxoimidazolidin-1- yl)methyl)pyridin-2- yl)amino)butanenitrile |
| 279 | 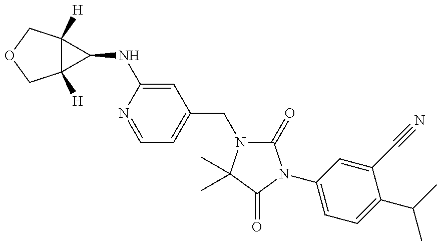 [SMILES: CC(C)c1ccc(N2C(=O)N(Cc3ccnc(N[C@H]4[C@@H]5COC[C@@H]54)c3)C(C)(C)C2=O)cc1C#N] | 5-(3-((2-(((1R,5S,6r)-3- oxabicyclo[3.1.0]hexan-6- yl)amino)pyridin-4-yl)methyl)- 4,4-dimethyl-2,5- dioxoimidazolidin-1-yl)-2- isopropylbenzonitrile |
| 280 | 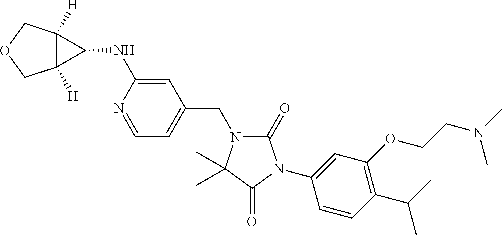 [SMILES: CC(C)c1ccc(N2C(=O)N(Cc3ccnc(N[C@H]4[C@@H]5COC[C@@H]54)c3)C(C)(C)C2=O)cc1OCCN(C)C] | 1-((2-(((1R,5S,6r)-3- oxabicyclo[3.1,0]hexan-6- yl)amino)pyridin-4-yl)methyl)- 3-(3-(2- (dimethylamino)ethoxy)-4- isopropylphenyl)-5,5- dimethylimidazolidine-2,4- dione |

In some embodiments, the compound is a pharmaceutically acceptable salt of a compound described in Table 6.

TABLE 7

| Cmpd | | |
|---|---|---|
| # | Structure | Name |
| 281 | 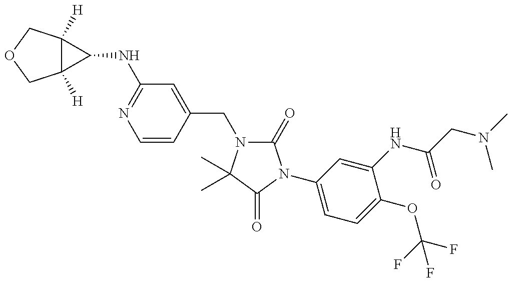 [SMILES: CN(C)CC(=O)Nc1cc(N2C(=O)N(Cc3ccnc(N[C@H]4[C@@H]5COC[C@@H]54)c3)C(C)(C)C2=O)ccc1OC(F)(F)F] | N-(5-(3-((2-(((1R,5S,6r)-3- oxabicyclo[3.1.0]hexan-6-yl)amino) pyridin-4-yl)methyl)-4,4-dimethyl- 2.5-dioxoimidazolidin-1-yl)-2- (trifluoromethoxy)phenyl)-2- (dimethylamino)acetamide |
| 282 | 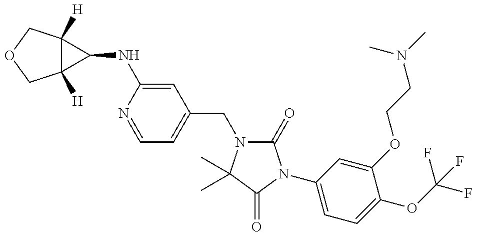 [SMILES: CN(C)CCOc1cc(N2C(=O)N(Cc3ccnc(N[C@H]4[C@@H]5COC[C@@H]54)c3)C(C)(C)C2=O)ccc1OC(F)(F)F] | 1-((2-(((1R,5S,6r)-3- oxabicyclo[3.1.0]hexan-6- yl)amino)pyridin-4-yl)methyl)-3-(3- (2-(dimethylamino)ethoxy)-4- (trifluoromethoxy)phenyl)-5,5- dimethylimidazolidine-2,4-dione |
| 283 | 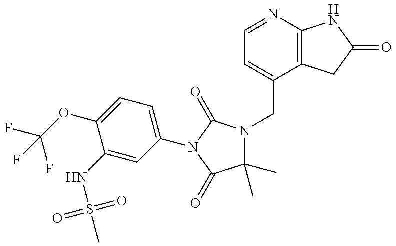 [SMILES: CC1(C)C(=O)N(c2ccc(OC(F)(F)F)c(NS(C)(=O)=O)c2)C(=O)N1Cc1ccnc2c1CC(=O)N2] | N-(5-(4,4-dimethyl-2,5-dioxo-3-((2- oxo-2,3-dihydro-1H-pyrrolo[2,3- b]pyridin-4-yl)methyl)imidazolidin-1- yl)-2-(trifluoromethoxy)phenyl) methanesulfonamide |
| 284 | 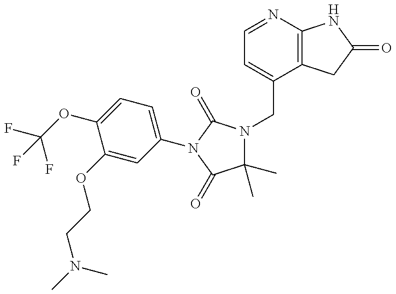 [SMILES: CN(C)CCOc1cc(N2C(=O)N(Cc3ccnc4c3CC(=O)N4)C(C)(C)C2=O)ccc1OC(F)(F)F] | 3-(3-(2-(dimethylamino)ethoxy)-4- (trifluoromethoxy)phenyl)-5,5- dimethyl-1-((2-oxo-2,3-dihydro-1H- pyrrolo[2,3-b]pyridin-4- yl)methyl)imidazolidine-2,4-dione |
| 285 | 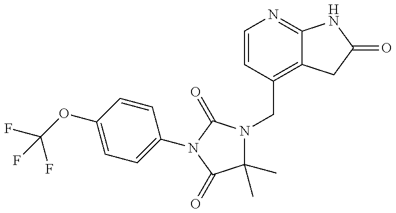 [SMILES: CC1(C)C(=O)N(c2ccc(OC(F)(F)F)cc2)C(=O)N1Cc1ccnc2c1CC(=O)N2] | 5,5-dimethyl-1-((2-oxo-2,3-dihydro- 1H-pyrrolo[2,3-b]pyridin-4- yl)methyl)-3-(4- (trifluoromethoxy)phenyl) imidazolidine-2,4-dione |
| 286 | 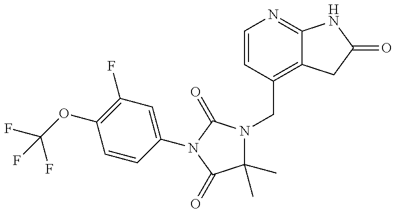 [SMILES: CC1(C)C(=O)N(c2ccc(OC(F)(F)F)c(F)c2)C(=O)N1Cc1ccnc2c1CC(=O)N2] | 3-(3-fluoro-4- (trifluoromethoxy)phenyl)-5,5- dimethyl-1-((2-oxo-2,3-dihydro-1H- pyrrolo[2,3-b]pyridin-4- yl)methyl)imidazolidine-2,4-dione |
| 287 | 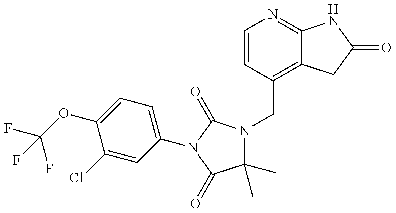 [SMILES: CC1(C)C(=O)N(c2ccc(OC(F)(F)F)c(Cl)c2)C(=O)N1Cc1ccnc2c1CC(=O)N2] | 3-(3-chloro-4- (trifluoromethoxy)phenyl)-5,5- dimethyl-1-((2-oxo-2,3-dihydro-1H- pyrrolo[2,3-b]pyridin-4- yl)methyl)imidazolidine-2,4-dione |
| 288 | 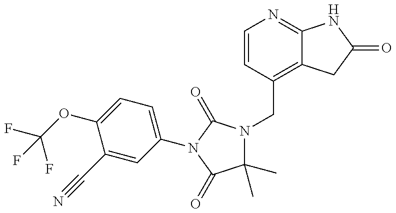 [SMILES: CC1(C)C(=O)N(c2ccc(OC(F)(F)F)c(C#N)c2)C(=O)N1Cc1ccnc2c1CC(=O)N2] | 5-(4,4-dimethyl-2,5-dioxo-3-((2-oxo- 2,3-dihydro-1H-pyrrolo[2,3- b]pyridin-4-yl)methyl)imidazolidin-1- yl)-2-(trifluoromethoxy)benzonitrile |
| 54 | 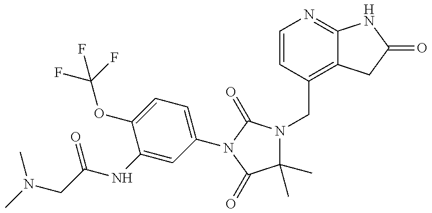 [SMILES: CN(C)CC(=O)Nc1cc(N2C(=O)N(Cc3ccnc4c3CC(=O)N4)C(C)(C)C2=O)ccc1OC(F)(F)F] | N-(5-(4,4-dimethyl-2,5-dioxo-3-((2- oxo-2,3-dihydro-1H-pyrrolo[2,3- b]pyridin-4-yl)methyl)imidazolidin-1- yl)-2-(trifluoromethoxy)phenyl)-2- (dimethylamino)acetamide |
| 74 | 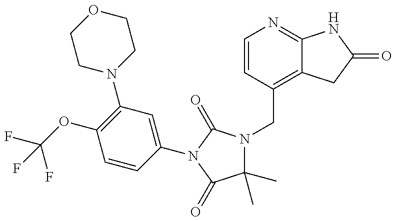 [SMILES: CC1(C)C(=O)N(c2ccc(OC(F)(F)F)c(N3CCOCC3)c2)C(=O)N1Cc1ccnc2c1CC(=O)N2] | 5,5-dimethyl-3-(3-morpholino-4- (trifluoromethoxy)phenyl)-1-((2-oxo- 2,3-dihydro-1H-pyrrolo[2,3- b]pyridin-4-yl)methyl)imidazolidine- 2,4-dione |
| 75 | 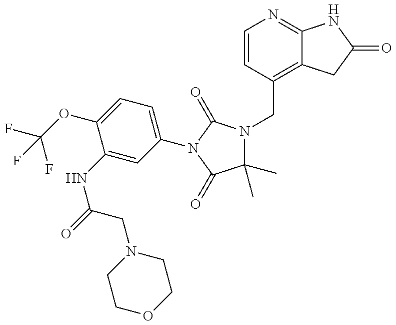 [SMILES: CC1(C)C(=O)N(c2ccc(OC(F)(F)F)c(NC(=O)CN3CCOCC3)c2)C(=O)N1Cc1ccnc2c1CC(=O)N2] | N-(5-(4,4-dimethyl-2,5-dioxo-3-((2- oxo-2,3-dihydro-1H-pyrrolo[2,3- b]pyridin-4-yl)methyl)imidazolidin-1- yl)-2-(trifluoromethoxy)phenyl)-2- morpholinoacetamide |

In some embodiments, the compound is a pharmaceutically acceptable salt of a compound described in Table 7.

TABLE 8

| Cmpd | | |
|---|---|---|
| # | Structure | Name |
| 289 | 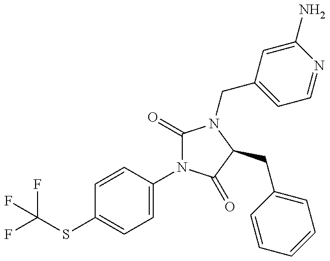 [SMILES: Nc1cc(CN2C(=O)N(c3ccc(SC(F)(F)F)cc3)C(=O)[C@@H]2Cc2ccccc2)ccn1] | (S)-1-((2-aminopyridin-4-yl)methyl)-5-benzyl- 3-(4- ((trifluoromethyl)thio)phenyl)imidazolidine-2,4- dione |
| 290 | 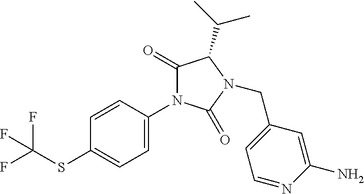 [SMILES: CC(C)[C@H]1C(=O)N(c2ccc(SC(F)(F)F)cc2)C(=O)N1Cc1ccnc(N)c1] | (S)-1-((2-aminopyridin-4-yl)methyl)-5- isopropyl-3-(4- ((trifluoromethyl)thio)phenyl)imidazolidine-2,4- dione |
| 291 | 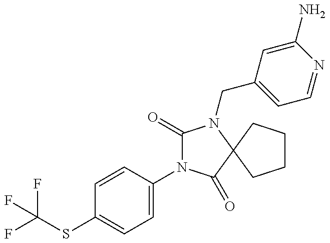 [SMILES: Nc1cc(CN2C(=O)N(c3ccc(SC(F)(F)F)cc3)C(=O)C23CCCC3)ccn1] | 1-(2-aminopyridin-4-yl)methyl)-3-(4- ((trifluoromethyl)thio)phenyl)-1,3- diazaspiro[4.4]nonane-2,4-dione |
| 292 | 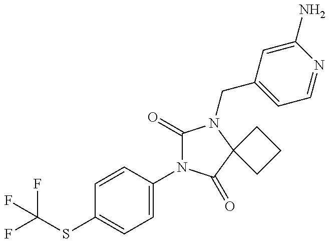 [SMILES: Nc1cc(CN2C(=O)N(c3ccc(SC(F)(F)F)cc3)C(=O)C23CCC3)ccn1] | 5-((2-aminopyridin-4-yl)methyl)-7-(4- ((trifluoromethyl)thio)phenyl)-5,7- diazaspiro[3.4]octane-6,8-dione |
| 293 | 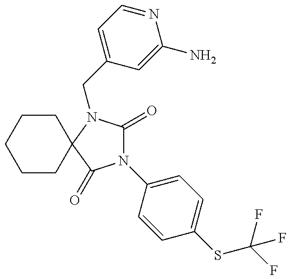 [SMILES: Nc1cc(CN2C(=O)N(c3ccc(SC(F)(F)F)cc3)C(=O)C23CCCCC3)ccn1] | 1-((2-aminopyridin-4-yl)methyl)-3-(4- ((trifluoromethyl)thio)phenyl)-1,3- diazaspiro[4.5]decane-2,4-dione |
| 294 | 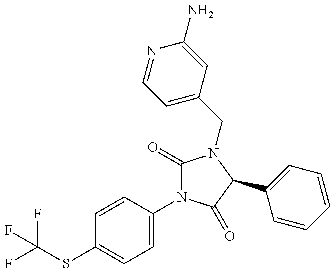 [SMILES: Nc1cc(CN2C(=O)N(c3ccc(SC(F)(F)F)cc3)C(=O)[C@@H]2c2ccccc2)ccn1] | (S)-1-((2-aminopyridin-4-yl)methyl)-5-phenyl- 3-(4- ((trifluoromethyl)thio)phenyl)imidazolidine-2,4- dione |
| 295 | 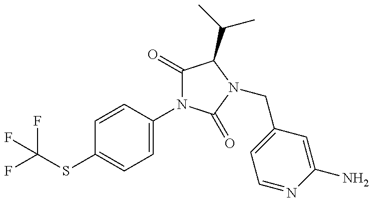 [SMILES: CC(C)[C@@H]1C(=O)N(c2ccc(SC(F)(F)F)cc2)C(=O)N1Cc1ccnc(N)c1] | (R)-1-((2-aminopyridin-4-yl)methyl)-5- isopropyl-3-(4- ((trifluoromethyl)thio)phenyl)imidazolidine-2,4- dione |
| 296 | 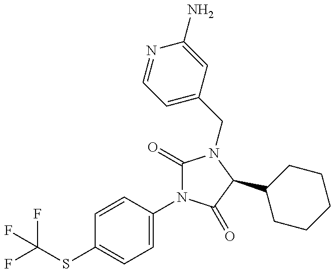 [SMILES: Nc1cc(CN2C(=O)N(c3ccc(SC(F)(F)F)cc3)C(=O)[C@@H]2C2CCCCC2)ccn1] | (S)-1-((2-aminopyridin-4-yl)methyl)-5- cyclohexyl-3-(4- ((trifluoromethyl)thio)phenyl)imidazolidine-2,4- dione |
| 297 | 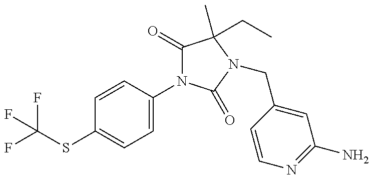 [SMILES: CCC1(C)C(=O)N(c2ccc(SC(F)(F)F)cc2)C(=O)N1Cc1ccnc(N)c1] | 1-((2-aminopyridin-4-yl)methyl)-5-ethyl-5- methyl-3-(4- ((trifluoromethyl)thio)phenyl)imidazolidine-2,4- dione |
| 298 | 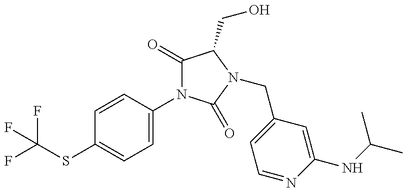 [SMILES: CC(C)Nc1cc(CN2C(=O)N(c3ccc(SC(F)(F)F)cc3)C(=O)[C@@H]2CO)ccn1] | (S)-5-(hydroxymethyl)-1-((2- (isopropylamino)pyridin-4-yl)methyl)-3-(4- ((trifluoromethyl)thio)phenyl)imidazolidine-2,4- dione |
| 299 | 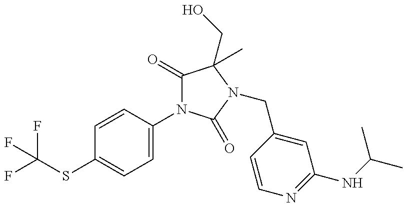 [SMILES: CC(C)Nc1cc(CN2C(=O)N(c3ccc(SC(F)(F)F)cc3)C(=O)C2(C)CO)ccn1] | 5-(hydroxymethyl)-1-((2- (isopropylamino)pyridin-4-yl)methyl)-5-methyl- 3-(4- ((trifluoromethyl)thio)phenyl)imidazolidine-2,4- dione |
| 300 | 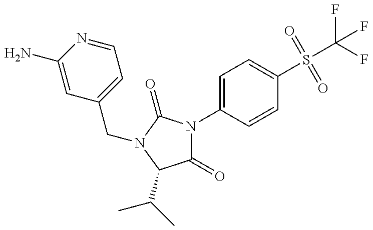 [SMILES: CC(C)[C@H]1C(=O)N(c2ccc(S(=O)(=O)C(F)(F)F)cc2)C(=O)N1Cc1ccnc(N)c1] | (S)-1-((2-aminopyridin-4-yl)methyl)-5- isopropyl-3-(4- ((trifluoromethyl)sulfonyl)phenyl)imidazolidine- 2,4-dione |

In some embodiments, the compound is a pharmaceutically acceptable salt of a compound described in Table 8.

TABLE 9

| Cmpd # | Structure | Name |
|---|---|---|
| 301 | 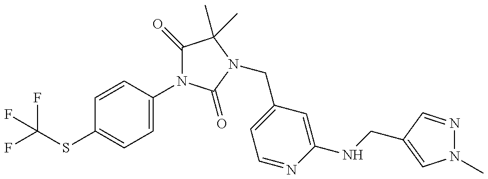 [SMILES: Cn1cc(CNc2cc(CN3C(=O)N(c4ccc(SC(F)(F)F)cc4)C(=O)C3(C)C)ccn2)cn1] | 5,5-dimethyl-1-((2-(((1-methyl-1H- pyrazol-4-yl)methyl)amino)pyridin- 4-yl)methyl)-3-(4- ((trifluoromethyl)thio)phenyl) imidazolidine-2,4-dione |
| 302 | 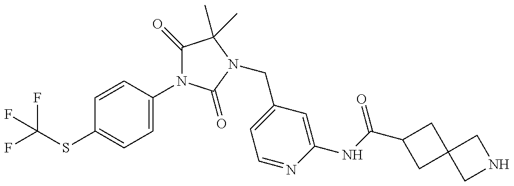 [SMILES: CC1(C)C(=O)N(c2ccc(SC(F)(F)F)cc2)C(=O)N1Cc1ccnc(NC(=O)C2CC3(CNC3)C2)c1] | N-(4-((5,5-dimethyl-2,4-dioxo-3-(4- ((trifluoromethyl)thio)phenyl) imidazolidin-1-yl) methyl)pyridin-2-yl)-2- azaspiro[3.3]heptane-6-carboxamide |
| 303 | 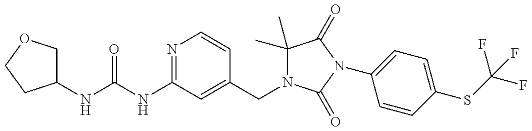 [SMILES: CC1(C)C(=O)N(c2ccc(SC(F)(F)F)cc2)C(=O)N1Cc1ccnc(NC(=O)NC2CCOC2)c1] | 1-(4-((5,5-dimethyl-2,4-dioxo-3-(4- ((trifluoromethyl)thio)phenyl) imidazolidin-1-yl) methyl)pyridin-2-yl)-3- (tetrahydrofuran-3-yl)urea |
| 304 | 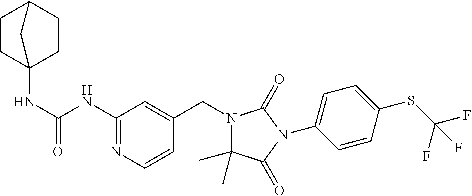 [SMILES: CC1(C)C(=O)N(c2ccc(SC(F)(F)F)cc2)C(=O)N1Cc1ccnc(NC(=O)NC23CCC(CC2)C3)c1] | 1-(bicyclo[2.2.1]heptan-1-yl)-3-(4- ((5,5-dimethyl-2,4-dioxo-3-(4- ((trifluoromethyl)thio)phenyl) imidazolidin-1-yl)methyl) pyridin-2-yl)urea |
| 305 | 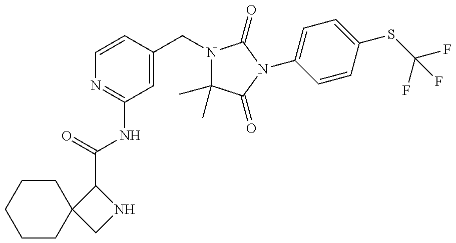 [SMILES: CC1(C)C(=O)N(c2ccc(SC(F)(F)F)cc2)C(=O)N1Cc1ccnc(NC(=O)C2NCC23CCCCC3)c1] | N-(4-((5,5-dimethyl-2,4-dioxo-3-(4- ((trifluoromethyl)thio)phenyl) imidazolidin-1-yl)methyl) pyridin-2-yl)-2- azaspiro[3.5]nonane-1-carboxamide |
| 306 | 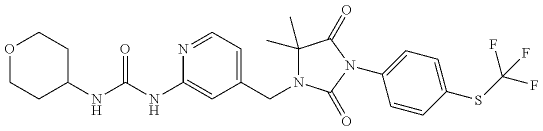 [SMILES: CC1(C)C(=O)N(c2ccc(SC(F)(F)F)cc2)C(=O)N1Cc1ccnc(NC(=O)NC2CCOCC2)c1] | 1-(4-((5,5-dimethyl-2,4-dioxo-3-(4- ((trifluoromethyl)thio)phenyl) imidazolidin-1-yl)methyl) pyridin-2-yl)-3- (tetrahydro-2H-pyran-4-yl)urea |
| 307 | 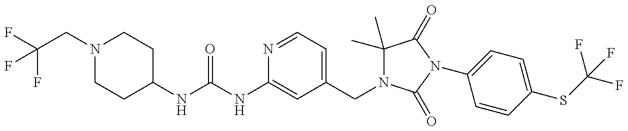 [SMILES: CC1(C)C(=O)N(c2ccc(SC(F)(F)F)cc2)C(=O)N1Cc1ccnc(NC(=O)NC2CCN(CC(F)(F)F)CC2)c1] | 1-(4-((5,5-dimethyl-2,4-dioxo-3-(4- ((trifluoromethyl)thio)phenyl) imidazolidin-1-yl)methyl) pyridin-2-yl)-3- (1-(2,2,2-trifluoroethyl)piperidin-4- yl)urea |
| 308 | 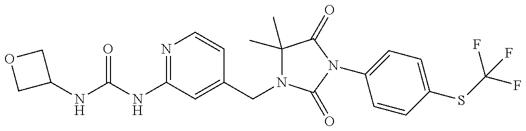 [SMILES: CC1(C)C(=O)N(c2ccc(SC(F)(F)F)cc2)C(=O)N1Cc1ccnc(NC(=O)NC2COC2)c1] | 1-(4-((5,5-dimethyl-2,4-dioxo-3-(4- ((trifluoromethyl)thio)phenyl) imidazolidin-1-yl)methyl) pyridin-2-yl)-3- (oxetan-3-yl)urea |
| 309 | 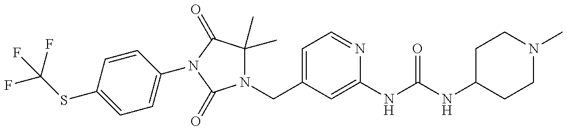 [SMILES: CN1CCC(NC(=O)Nc2cc(CN3C(=O)N(c4ccc(SC(F)(F)F)cc4)C(=O)C3(C)C)ccn2)CC1] | 1-(4-((5,5-dimethyl-2,4-dioxo-3-(4- ((trifluoromethyl)thio)phenyl) imidazolidin-1-yl)methyl) pyridin-2-yl)-3- (1-methylpiperidin-4-yl)urea |
| 310 | 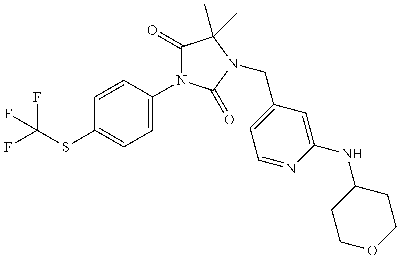 [SMILES: CC1(C)C(=O)N(c2ccc(SC(F)(F)F)cc2)C(=O)N1Cc1ccnc(NC2CCOCC2)c1] | 5,5-dimethyl-1-((2-((tetrahydro-2H- pyran-4-yl)amino)pyridin-4- yl)methyl)-3-(4- ((trifluoromethyl)thio)phenyl) imidazolidine-2,4-dione |
| 311 | 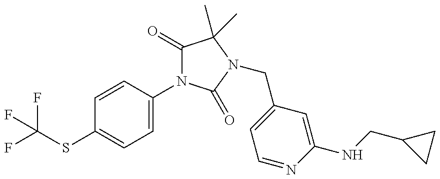 [SMILES: CC1(C)C(=O)N(c2ccc(SC(F)(F)F)cc2)C(=O)N1Cc1ccnc(NCC2CC2)c1] | 1-((2- ((cyclopropylmethyl)amino)pyridin- 4-yl)methyl)-5,5-dimethyl-3-(4- ((trifluoromethyl)thio)phenyl) imidazolidine-2,4-dione |
| 312 | 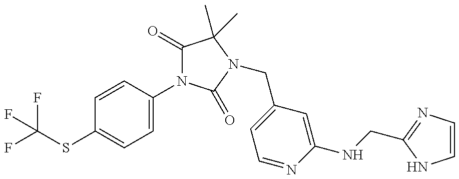 [SMILES: CC1(C)C(=O)N(c2ccc(SC(F)(F)F)cc2)C(=O)N1Cc1ccnc(NCc2ncc[nH]2)c1] | 1-((2-(((1H-imidazol-2- yl)methyl)amino)pyridin-4- yl)methyl)-5,5-dimethyl-3-(4- ((trifluoromethyl)thio)phenyl) imidazolidine-2,4-dione |
| 313 | 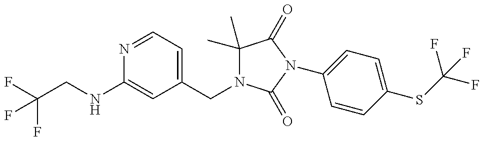 [SMILES: CC1(C)C(=O)N(c2ccc(SC(F)(F)F)cc2)C(=O)N1Cc1ccnc(NCC(F)(F)F)c1] | 5,5-dimethyl-1-((2-((2,2,2- trifluoroethyl)amino)pyridin-4- yl)methyl)-3-(4- ((trifluoromethyl)thio)phenyl) imidazolidine-2,4-dione |
| 314 | 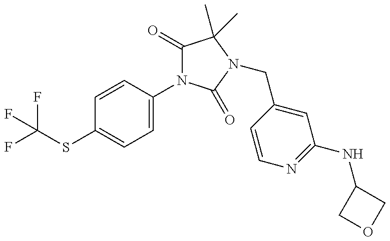 [SMILES: CC1(C)C(=O)N(c2ccc(SC(F)(F)F)cc2)C(=O)N1Cc1ccnc(NC2COC2)c1] | 5,5-dimethyl-1-((2-(oxetan-3- ylamino)pyridin-4-yl)methyl)-3-(4- ((trifluoromethyl)thio)phenyl) imidazolidine-2,4-dione |
| 315 | 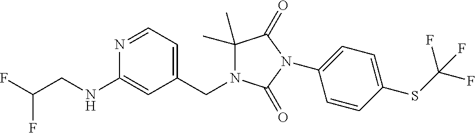 [SMILES: CC1(C)C(=O)N(c2ccc(SC(F)(F)F)cc2)C(=O)N1Cc1ccnc(NCC(F)F)c1] | 1-((2-((2,2- difluoroethyl)amino)pyridin-4- yl)methyl)-5,5-dimethyl-3-(4- ((trifluoromethyl)thio)phenyl) imidazolidine-2,4-dione |
| 316 | 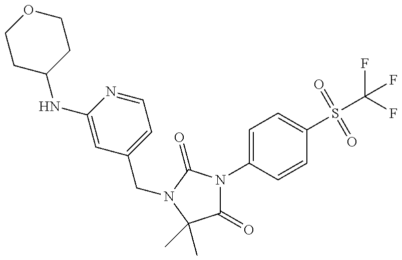 [SMILES: CC1(C)C(=O)N(c2ccc(S(=O)(=O)C(F)(F)F)cc2)C(=O)N1Cc1ccnc(NC2CCOCC2)c1] | 5,5-dimethyl-1-((2-((tetrahydro-2H- pyran-4-yl)amino)pyridin-4- yl)methyl)-3-(4- ((trifluoromethyl)sulfonyl)phenyl) imidazolidine-2,4-dione |
| 317 | 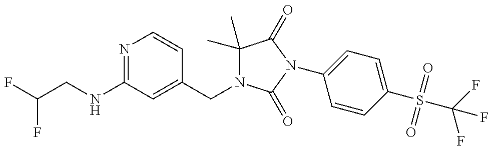 [SMILES: CC1(C)C(=O)N(c2ccc(S(=O)(=O)C(F)(F)F)cc2)C(=O)N1Cc1ccnc(NCC(F)F)c1] | 1-((2-((2,2- difluoroethyl)amino)pyridin-4- yl)methyl)-5,5-dimethyl-3-(4- ((trifluoromethyl)sulfonyl)phenyl) imidazolidine-2,4-dione |
| 318 | 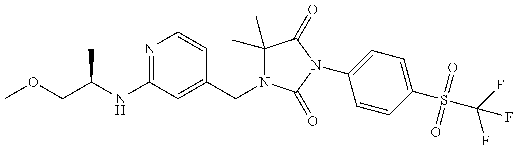 [SMILES: COC[C@@H](C)Nc1cc(CN2C(=O)N(c3ccc(S(=O)(=O)C(F)(F)F)cc3)C(=O)C2(C)C)ccn1] | (R)-1-((2-((1-methoxypropan-2- yl)amino)pyridin-4-yl)methyl)-5,5- dimethyl-3-(4- ((trifluoromethyl)sulfonyl)phenyl) imidazolidine-2,4-dione |
| 319 | 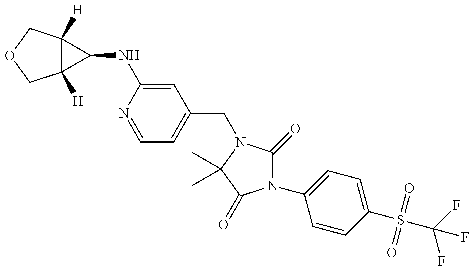 [SMILES: CC1(C)C(=O)N(c2ccc(S(=O)(=O)C(F)(F)F)cc2)C(=O)N1Cc1ccnc(N[C@H]2[C@@H]3COC[C@@H]32)c1] | 1-((2-(((1R,5S,6r)-3- oxabicyclo[3.1.0]hexan-6- yl)amino)pyridin-4-yl)methyl)-5,5- dimethyl-3-(4- ((trifluoromethyl)sulfonyl)phenyl) imidazolidine-2,4-dione |
| 320 | 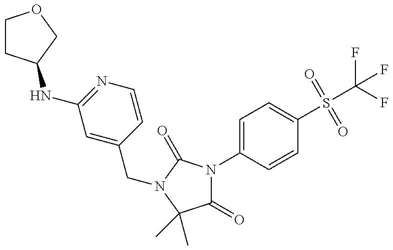 [SMILES: CC1(C)C(=O)N(c2ccc(S(=O)(=O)C(F)(F)F)cc2)C(=O)N1Cc1ccnc(N[C@H]2CCOC2)c1] | (S)-5,5-dimethyl-1-((2- ((tetrahydrofuran-3- yl)amino)pyridin-4-yl)methyl)-3-(4- ((trifluoromethyl)sulfonyl)phenyl) imidazolidine-2,4-dione |
| 321 | 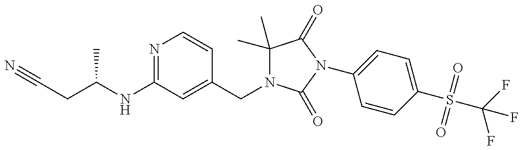 [SMILES: C[C@@H](CC#N)Nc1cc(CN2C(=O)N(c3ccc(S(=O)(=O)C(F)(F)F)cc3)C(=O)C2(C)C)ccn1] | (S)-3-((4-((5,5-dimethyl-2,4-dioxo- 3-(4- ((trifluoromethyl)sulfonyl)phenyl) imidazolidin-1-yl)methyl)pyridin-2- yl)amino)butanenitrile |
| 322 | 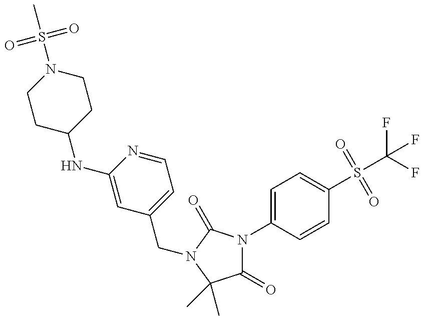 [SMILES: CC1(C)C(=O)N(c2ccc(S(=O)(=O)C(F)(F)F)cc2)C(=O)N1Cc1ccnc(NC2CCN(S(C)(=O)=O)CC2)c1] | 5,5-dimethyl-1-((2-((1- (methylsulfonyl)piperidin-4- yl)amino)pyridin-4-yl)methyl)-3-(4- ((trifluoromethyl)sulfonyl)phenyl) imidazolidine-2,4-dione |
| 323 | 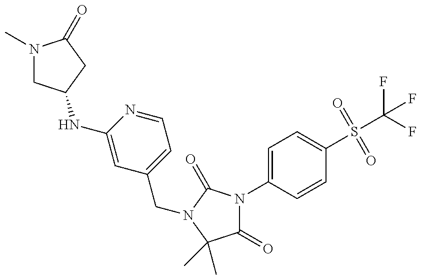 [SMILES: CN1C[C@@H](Nc2cc(CN3C(=O)N(c4ccc(S(=O)(=O)C(F)(F)F)cc4)C(=O)C3(C)C)ccn2)CC1=O] | (S)-5,5-dimethyl-1-((2-((1-methyl- 5-oxopyrrolidin-3-yl)amino)pyridin- 4-yl)methyl)-3-(4- ((trifluoromethyl)sulfonyl)phenyl) imidazolidine-2,4-dione |
| 324 | 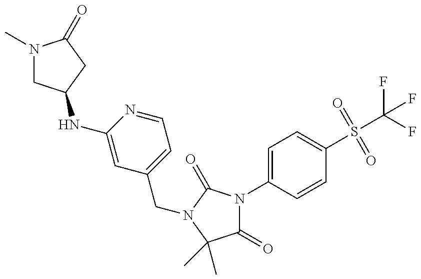 [SMILES: CN1C[C@H](Nc2cc(CN3C(=O)N(c4ccc(S(=O)(=O)C(F)(F)F)cc4)C(=O)C3(C)C)ccn2)CC1=O] | (R)-5,5-dimethyl-1-((2-((1-methyl- 5-oxopyrrolidin-3-yl)amino)pyridin- 4-yl)methyl)-3-(4- ((trifluoromethyl)sulfonyl)phenyl) imidazolidine-2,4-dione |
| 325 | 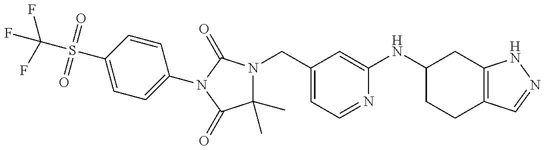 [SMILES: CC1(C)C(=O)N(c2ccc(S(=O)(=O)C(F)(F)F)cc2)C(=O)N1Cc1ccnc(NC2CCc3cn[nH]c3C2)c1] | 5,5-dimethyl-1-((2-((4,5,6,7- tetrahydro-1H-indazol-6- yl)amino)pyridin-4-yl)methyl)-3-(4- ((trifluoromethyl)sulfonyl)phenyl) imidazolidine-2,4-dione |
| 326 | 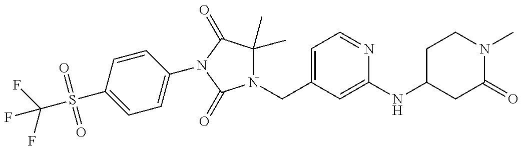 [SMILES: CN1CCC(Nc2cc(CN3C(=O)N(c4ccc(S(=O)(=O)C(F)(F)F)cc4)C(=O)C3(C)C)ccn2)CC1=O] | 5,5-dimethyl-1-((2-((1-methyl-2- oxopiperidin-4-yl)amino)pyridin-4- yl)methyl)-3-(4- ((trifluoromethyl)sulfonyl)phenyl) imidazolidine-2,4-dione |
| 327 | 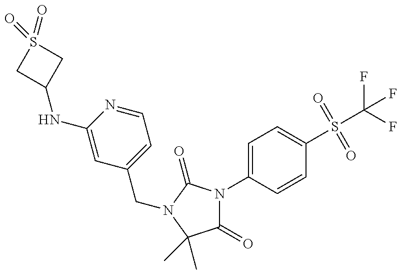 [SMILES: CC1(C)C(=O)N(c2ccc(S(=O)(=O)C(F)(F)F)cc2)C(=O)N1Cc1ccnc(NC2CS(=O)(=O)C2)c1] | 1-((2-((1,1-dioxidothietan-3- yl)amino)pyridin-4-yl)methyl)-5,5- dimethyl-3-(4- ((trifluoromethyl)sulfonyl)phenyl) imidazolidine-2,4-dione |
| 328 | 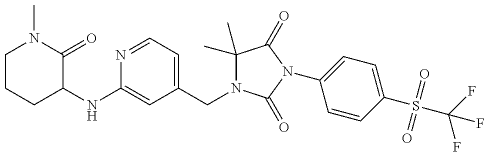 [SMILES: CN1CCCC(Nc2cc(CN3C(=O)N(c4ccc(S(=O)(=O)C(F)(F)F)cc4)C(=O)C3(C)C)ccn2)C1=O] | 5,5-dimethyl-1-((2-((1-methyl-2- oxopiperidin-3-yl)amino)pyridin-4- yl)methyl)-3-(4- ((trifluoromethyl)sulfonyl)phenyl) imidazolidine-2,4-dione |
| 329 | 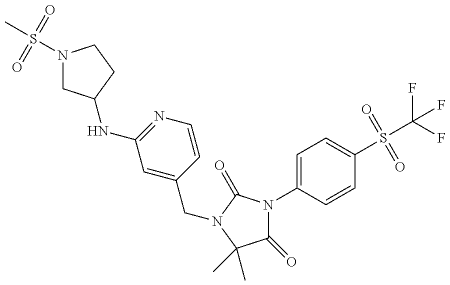 [SMILES: CC1(C)C(=O)N(c2ccc(S(=O)(=O)C(F)(F)F)cc2)C(=O)N1Cc1ccnc(NC2CCN(S(C)(=O)=O)C2)c1] | 5,5-dimethyl-1-((2-((1- (methylsulfonyl)pyrrolidin-3- yl)amino)pyridin-4-yl)methyl)-3-(4- ((trifluoromethyl)sulfonyl)phenyl) imidazolidine-2,4-dione |
| 330 | 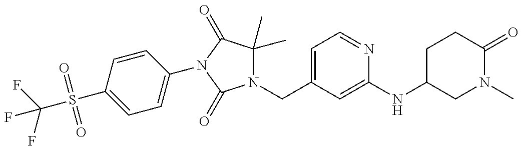 [SMILES: CN1CC(Nc2cc(CN3C(=O)N(c4ccc(S(=O)(=O)C(F)(F)F)cc4)C(=O)C3(C)C)ccn2)CCC1=O] | 5,5-dimethyl-1-((2-((1-methyl-6- oxopiperidin-3-yl)amino)pyridin-4- yl)methyl)-3-(4- ((trifluoromethyl)sulfonyl)phenyl) imidazolidine-2,4-dione |
| 331 | 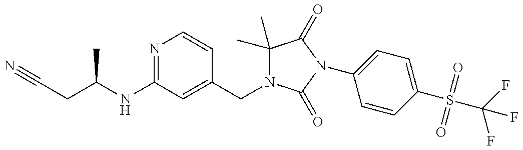 [SMILES: C[C@H](CC#N)Nc1cc(CN2C(=O)N(c3ccc(S(=O)(=O)C(F)(F)F)cc3)C(=O)C2(C)C)ccn1] | (R)-3-((4-((5,5-dimethyl-2,4-dioxo- 3-(4- ((trifluoromethyl)sulfonyl)phenyl) imidazolidin-1-yl)methyl)pyridin-2- yl)amino)butanenitrile |
| 332 | 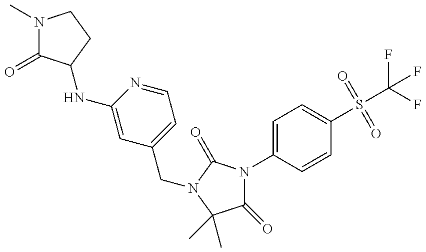 [SMILES: CN1CCC(Nc2cc(CN3C(=O)N(c4ccc(S(=O)(=O)C(F)(F)F)cc4)C(=O)C3(C)C)ccn2)C1=O] | 5,5-dimethyl-1-((2-((1-methyl-2- oxopyrrolidin-3-yl)amino)pyridin-4- yl)methyl)-3-(4- ((trifluoromethyl)sulfonyl)phenyl) imidazolidine-2,4-dione |
| 333 | 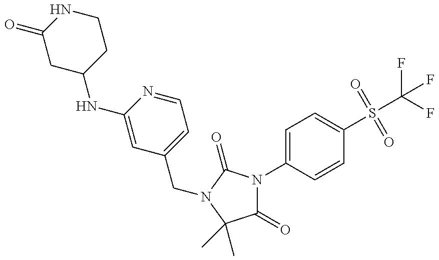 [SMILES: CC1(C)C(=O)N(c2ccc(S(=O)(=O)C(F)(F)F)cc2)C(=O)N1Cc1ccnc(NC2CCNC(=O)C2)c1] | 5,5-dimethyl-1-((2-((2-oxopiperidin- 4-yl)amino)pyridin-4-yl)methyl)-3- (4- ((trifluoromethyl)sulfonyl)phenyl) imidazolidine-2,4-dione |
| 334 | 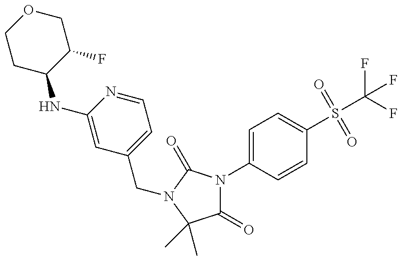 [SMILES: CC1(C)C(=O)N(c2ccc(S(=O)(=O)C(F)(F)F)cc2)C(=O)N1Cc1ccnc(N[C@H]2CCOC[C@@H]2F)c1] | 1-((2-(((3R,4S)-3-fluorotetrahydro- 2H-pyran-4-yl)amino)pyridin-4- yl)methyl)-5,5-dimethyl-3-(4- ((trifluoromethyl)sulfonyl)phenyl) imidazolidine-2,4-dione |
| 335 | 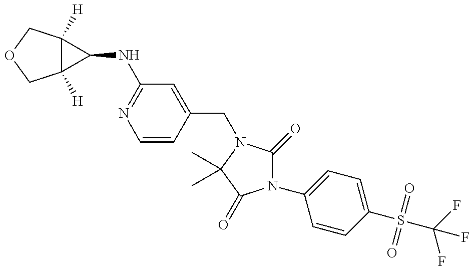 [SMILES: CC1(C)C(=O)N(c2ccc(S(=O)(=O)C(F)(F)F)cc2)C(=O)N1Cc1ccnc(N[C@@H]2[C@@H]3COC[C@@H]32)c1] | 1-((2-(((1R,5S,6s)-3- oxabicyclo[3.1.0]hexan-6- yl)amino)pyridin-4-yl)methyl)-5,5- dimethyl-3-(4- ((trifluoromethyl)sulfonyl)phenyl) imidazolidine-2,4-dione |
| 336 | 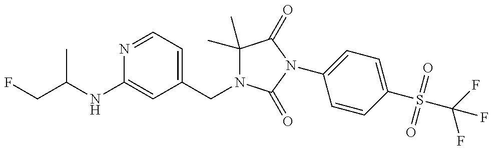 [SMILES: CC(CF)Nc1cc(CN2C(=O)N(c3ccc(S(=O)(=O)C(F)(F)F)cc3)C(=O)C2(C)C)ccn1] | 1-((2-((1-fluoropropan-2- yl)amino)pyridin-4-yl)methyl)-5,5- dimethyl-3-(4- ((trifluoromethyl)sulfonyl)phenyl) imidazolidine-2,4-dione |
| 337 | 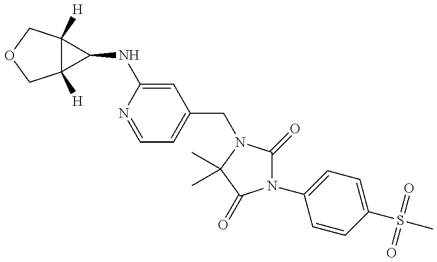 [SMILES: CC1(C)C(=O)N(c2ccc(S(C)(=O)=O)cc2)C(=O)N1Cc1ccnc(N[C@H]2[C@@H]3COC[C@@H]32)c1] | 1-((2-(((1R,SS,6r)-3- oxabicyclo[3.1.0]hexan-6- yl)amino)pyridin-4-yl)methyl)-5,5- dimethyl-3-(4- (methylsulfonyl)phenyl) imidazolidine-2,4-dione |
| 338 | 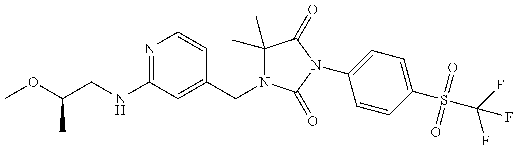 [SMILES: CO[C@H](C)CNc1cc(CN2C(=O)N(c3ccc(S(=O)(=O)C(F)(F)F)cc3)C(=O)C2(C)C)ccn1] | (R)-1-((2-((2- methoxypropyl)amino)pyridin-4- yl)methyl)-5,5-dimethyl-3-(4- ((trifluoromethyl)sulfonyl)phenyl) imidazolidine-2,4-dione |
| 339 | 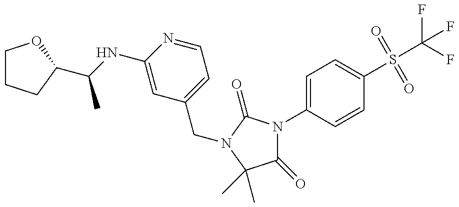 [SMILES: C[C@H](Nc1cc(CN2C(=O)N(c3ccc(S(=O)(=O)C(F)(F)F)cc3)C(=O)C2(C)C)ccn1)[C@@H]1CCCO1] | 5,5-dimethyl-1-((2-(((S)-1-((S)- tetrahydrofuran-2- yl)ethyl)amino)pyridin-4- yl)methyl)-3-(4- ((trifluoromethyl)sulfonyl)phenyl) imidazolidine-2,4-dione |
| 340 | 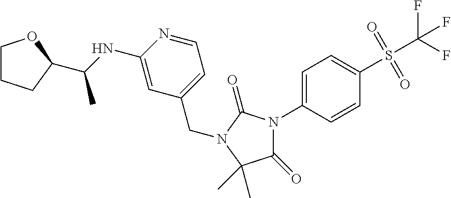 [SMILES: C[C@H](Nc1cc(CN2C(=O)N(c3ccc(S(=O)(=O)C(F)(F)F)cc3)C(=O)C2(C)C)ccn1)[C@H]1CCCO1] | 5,5-dimethyl-1-((2-(((S)-1-((R)- tetrahydrofuran-2- yl)ethyl)amino)pyridin-4- yl)methyl)-3-(4- ((trifluoromethyl)sulfonyl)phenyl) imidazolidine-2,4-dione |
| 341 | 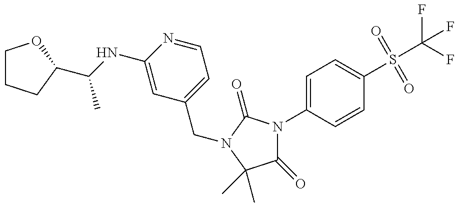 [SMILES: C[C@@H](Nc1cc(CN2C(=O)N(c3ccc(S(=O)(=O)C(F)(F)F)cc3)C(=O)C2(C)C)ccn1)[C@@H]1CCCO1] | 5,5-dimethyl-1-((2-(((R)-1-((S)- tetrahydrofuran-2- yl)ethyl)amino)pyridin-4- yl)methyl)-3-(4- ((trifluoromethyl)sulfonyl)phenyl) imidazolidine-2,4-dione |
| 342 | 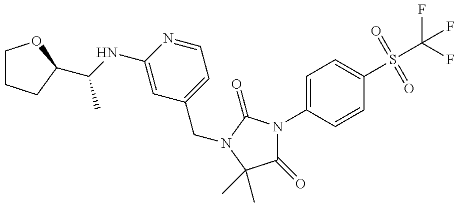 [SMILES: C[C@@H](Nc1cc(CN2C(=O)N(c3ccc(S(=O)(=O)C(F)(F)F)cc3)C(=O)C2(C)C)ccn1)[C@H]1CCCO1] | 5,5-dimethyl-1-((2-(((R)-1-((R)- tetrahydrofuran-2- yl)ethyl)amino)pyridin-4- yl)methyl)-3-(4- ((trifluoromethyl)sulfonyl)phenyl) imidazolidine-2,4-dione |
| 343 | 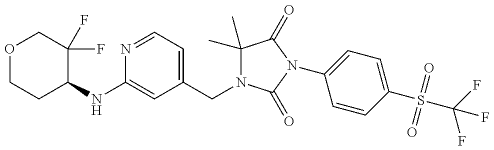 [SMILES: CC1(C)C(=O)N(c2ccc(S(=O)(=O)C(F)(F)F)cc2)C(=O)N1Cc1ccnc(N[C@H]2CCOCC2(F)F)c1] | (S)-1-((2-((3,3-difluorotetrahydro- 2H-pyran-4-yl)amino)pyridin-4- yl)methyl)-5,5-dimethyl-3-(4- ((trifluoromethyl)sulfonyl)phenyl) imidazolidine-2,4-dione |
| 344 | 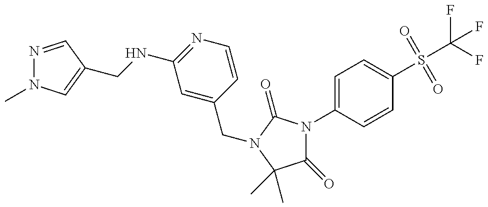 [SMILES: Cn1cc(CNc2cc(CN3C(=O)N(c4ccc(S(=O)(=O)C(F)(F)F)cc4)C(=O)C3(C)C)ccn2)cn1] | 5,5-dimethyl-1-((2-(((1-methyl-1H- pyrazol-4-yl)methyl)amino)pyridin- 4-yl)methyl)-3-(4- ((trifluoromethyl)sulfonyl)phenyl) imidazolidine-2,4-dione |

In some embodiments, the compound is a pharmaceutically acceptable salt of a compound described in Table 9.

TABLE 10

| Cmpd | | |
|---|---|---|
| # | Structure | Name |
| 345 | 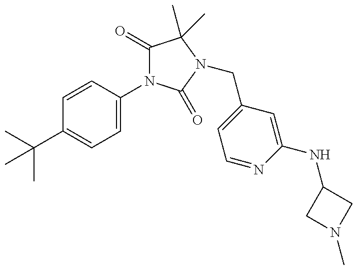 [SMILES: CN1CC(Nc2cc(CN3C(=O)N(c4ccc(C(C)(C)C)cc4)C(=O)C3(C)C)ccn2)C1] | 3-(4-(tert-butyl)phenyl)-5,5- dimethyl-1-((2-((1- methylazetidin-3- yl)amino)pyridin-4- yl)methyl)imidazolidine-2,4- dione |
| 346 | 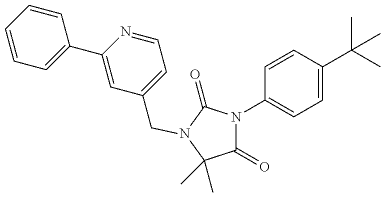 [SMILES: CC(C)(C)c1ccc(N2C(=O)N(Cc3ccnc(-c4ccccc4)c3)C(C)(C)C2=O)cc1] | 3-(4-(tert-butyl)phenyl)-5,5- dimethyl-1-((2-phenylpyridin-4-yl) methyl)imidazoline-2,4-dione |
| 347 | 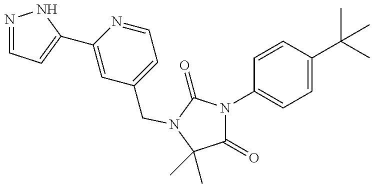 [SMILES: CC(C)(C)c1ccc(N2C(=O)N(Cc3ccnc(-c4ccn[nH]4)c3)C(C)(C)C2=O)cc1] | 1-((2-(1H-pyrazol-5-yl)pyridin- 4-yl)methyl)-3-(4-(tert- butyl)phenyl)-5,5- dimethylimidazoline-2,4-dione |
| 348 | 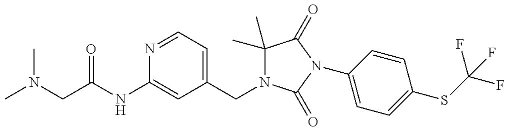 [SMILES: CN(C)CC(=O)Nc1cc(CN2C(=O)N(c3ccc(SC(F)(F)F)cc3)C(=O)C2(C)C)ccn1] | N-(4-((5,5-dimethyl-2,4-dioxo-3- (4-((trifluoromethyl)thio)phenyl) imidazolidin-1-yl)methyl)pyridin- 2-yl)-2-(dimethylamino)acetamide |
| 349 | 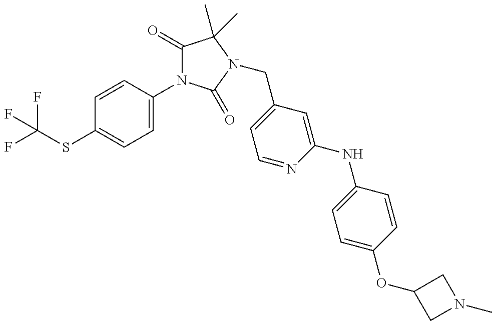 [SMILES: CN1CC(Oc2ccc(Nc3cc(CN4C(=O)N(c5ccc(SC(F)(F)F)cc5)C(=O)C4(C)C)ccn3)cc2)C1] | 5,5-dimethyl-1-((2-((4-((1- methylazetidin-3- yl)oxy)phenyl)amino)pyridin-4- yl)methyl)-3-(4- ((trifluoromethyl)thio)phenyl) imidazolidine-2,4-dione |
| 350 | 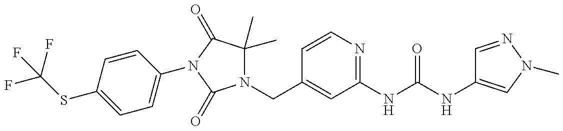 [SMILES: Cn1cc(NC(=O)Nc2cc(CN3C(=O)N(c4ccc(SC(F)(F)F)cc4)C(=O)C3(C)C)ccn2)cn1] | 1-(4-((5,5-dimethyl-2,4-dioxo-3- (4- ((trifluoromethyl)thio)phenyl) imidazolidin-1-yl)methyl)pyridin-2- yl)-3-(1-methyl-1H-pyrazol-4- yl)urea |
| 351 | 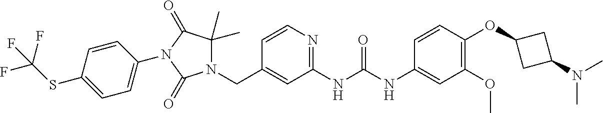 [SMILES: COc1cc(NC(=O)Nc2cc(CN3C(=O)N(c4ccc(SC(F)(F)F)cc4)C(=O)C3(C)C)ccn2)ccc1O[C@H]1C[C@@H](N(C)C)C1] | 1-(4-((5,5-dimethyl-2,4-dioxo-3- (4-((trifluoromethyl)thio)phenyl) imidazolidin-1-yl)methyl)pyridin- 2-yl)-3-(4-((1s,3s)-3- (dimethylamino)cyclobutoxy)-3- methoxyphenyl)urea |
| 352 | 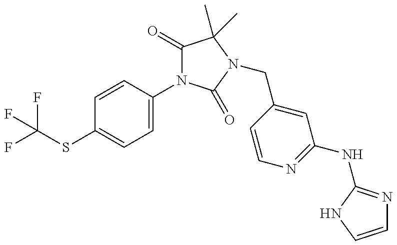 [SMILES: CC1(C)C(=O)N(c2ccc(SC(F)(F)F)cc2)C(=O)N1Cc1ccnc(Nc2ncc[nH]2)c1] | 1-((2-((1H-imidazol-2- yl)amino)pyridin-4-yl)methyl)- 5,5-dimethyl-3-(4- ((trifluoromethyl)thio)phenyl) imidazolidine-2,4-dione |
| 353 | 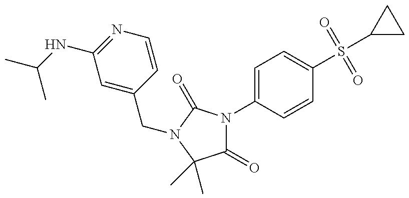 [SMILES: CC(C)Nc1cc(CN2C(=O)N(c3ccc(S(=O)(=O)C4CC4)cc3)C(=O)C2(C)C)ccn1] | 3-(4- (cyclopropylsulfonyl)phenyl)-1- ((2-(isopropylamino)pyridin-4- yl)methyl)-5,5- dimethylimidazolidine-2,4-dione |
| 354 | 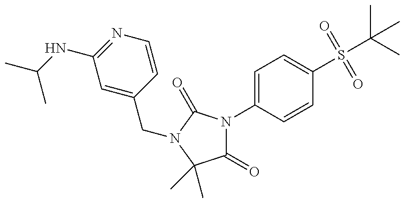 [SMILES: CC(C)Nc1cc(CN2C(=O)N(c3ccc(S(=O)(=O)C(C)(C)C)cc3)C(=O)C2(C)C)ccn1] | 3-(4-(tert-butylsulfonyl)phenyl)- 1-((2-(isopropylamino)pyridin-4- yl)methyl)-5,5- dimethylimidazolidine-2,4-dione |
| 355 | 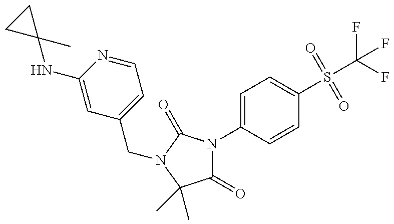 [SMILES: CC1(Nc2cc(CN3C(=O)N(c4ccc(S(=O)(=O)C(F)(F)F)cc4)C(=O)C3(C)C)ccn2)CC1] | 5,5-dimethyl-1-((2-((1- methylcyclopropyl)amino) pyridin-4-yl)methyl)-3-(4- ((trifluoromethyl)sulfonyl) phenyl)imidazolidine-2,4-dione |
| 356 | 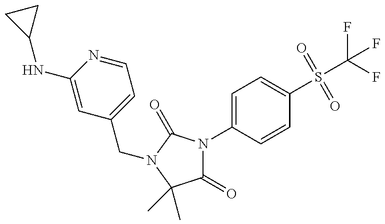 [SMILES: CC1(C)C(=O)N(c2ccc(S(=O)(=O)C(F)(F)F)cc2)C(=O)N1Cc1ccnc(NC2CC2)c1] | 1-((2- (cyclopropylamino)pyridin-4- yl)methyl)-5,5-dimethyl-3-(4- ((trifluoromethyl)sulfonyl) phenyl)imidazolidine-2,4-dione |
| 357 | 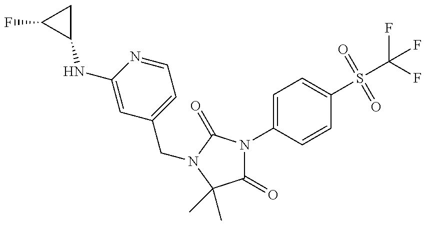 [SMILES: CC1(C)C(=O)N(c2ccc(S(=O)(=O)C(F)(F)F)cc2)C(=O)N1Cc1ccnc(N[C@H]2C[C@H]2F)c1] | 1-((2-(((1S,2R)-2- fluorocyclopropyl)amino)pyridin- 4-yl)methyl)-5,5-dimethyl-3-(4- ((trifluoromethyl)sulfonyl) phenyl)imidazolidine-2,4-dione |
| 358 | 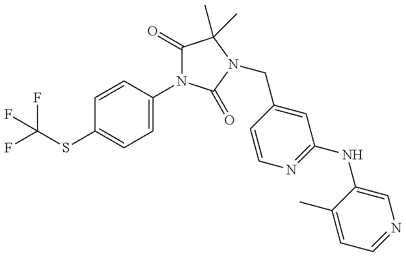 [SMILES: Cc1ccncc1Nc1cc(CN2C(=O)N(c3ccc(SC(F)(F)F)cc3)C(=O)C2(C)C)ccn1] | 5,5-dimethyl-1-((2-((4- methylpyridin-3- yl)amino)pyridin-4-yl)methyl)-3- (4- ((trifluoromethyl)thio)phenyl) imidazolidine-2,4-dione |
| 359 | 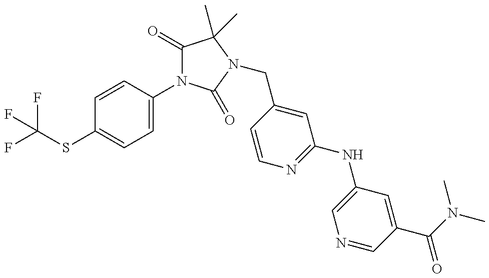 [SMILES: CN(C)C(=O)c1cncc(Nc2cc(CN3C(=O)N(c4ccc(SC(F)(F)F)cc4)C(=O)C3(C)C)ccn2)c1] | 5-((4-((5,5-dimethyl-2,4-dioxo- 3-(4- ((trifluoromethyl)thio)phenyl) imidazolidin-1-yl)methyl)pyridin-2- yl)amino)-N,N- dimethylnicotinamide |
| 360 | 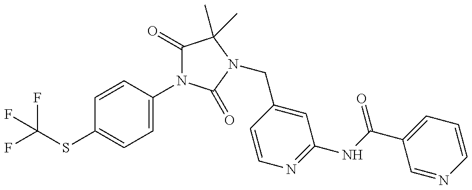 [SMILES: CC1(C)C(=O)N(c2ccc(SC(F)(F)F)cc2)C(=O)N1Cc1ccnc(NC(=O)c2cccnc2)c1] | N-(4-((5,5-dimethyl-2,4-dioxo-3- (4- ((trifluoromethyl)thio)phenyl) imidazolidin-1-yl)methyl)pyridin-2- yl)nicotinamide |
| 361 | 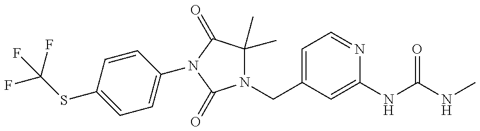 [SMILES: CNC(=O)Nc1cc(CN2C(=O)N(c3ccc(SC(F)(F)F)cc3)C(=O)C2(C)C)ccn1] | 1-(4-((5,5-dimethyl-2,4-dioxo-3- (4- ((trifluoromethyl)thio)phenyl) imidazolidin-1-yl)methyl)pyridin-2- yl)-3-methylurea |
| 362 | 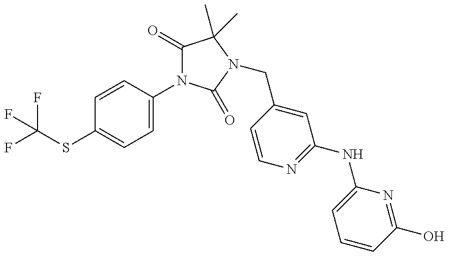 [SMILES: CC1(C)C(=O)N(c2ccc(SC(F)(F)F)cc2)C(=O)N1Cc1ccnc(Nc2cccc(O)n2)c1] | 1-((2-((6-hydroxypyridin-2- yl)amino)pyridin-4-yl)methyl)- 5,5-dimethyl-3-(4- ((trifluoromethyl)thio)phenyl) imidazolidine-2,4-dione |
| 363 | 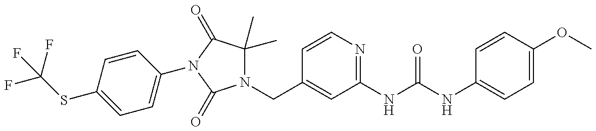 [SMILES: COc1ccc(NC(=O)Nc2cc(CN3C(=O)N(c4ccc(SC(F)(F)F)cc4)C(=O)C3(C)C)ccn2)cc1] | 1-(4-((5,5-dimethyl-2,4-dioxo-3- (4- ((trifluoromethyl)thio)phenyl) imidazolidin-1-yl)methyl)pyridin-2- yl)-3-(4-methoxyphenyl)urea |
| 364 | 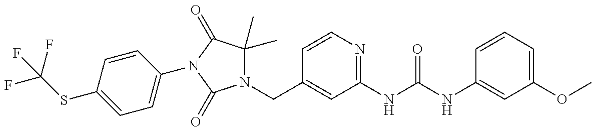 [SMILES: COc1cccc(NC(=O)Nc2cc(CN3C(=O)N(c4ccc(SC(F)(F)F)cc4)C(=O)C3(C)C)ccn2)c1] | 1-(4-((5,5-dimethyl-2,4-dioxo-3- (4- ((trifluoromethyl)thio)phenyl) imidazolidin-1-yl)methyl)pyridin-2- yl)-3-(3-methoxyphenyl)urea |
| 365 | 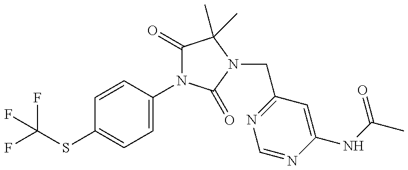 [SMILES: CC(=O)Nc1cc(CN2C(=O)N(c3ccc(SC(F)(F)F)cc3)C(=O)C2(C)C)ncn1] | N-(6-((5,5-dimethyl-2,4-dioxo-3- (4- ((trifluoromethyl)thio)phenyl) imidazolidin-1-yl)methyl)pyrimidin- 4-yl)acetamide |
| 366 | 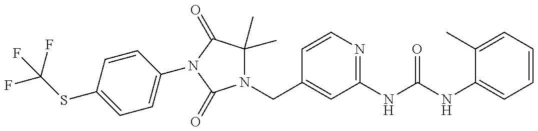 [SMILES: Cc1ccccc1NC(=O)Nc1cc(CN2C(=O)N(c3ccc(SC(F)(F)F)cc3)C(=O)C2(C)C)ccn1] | 1-(4-((5,5-dimethyl-2,4-dioxo-3- (4- ((trifluoromethyl)thio)phenyl) imidazolidin-1-yl)methyl)pyridin- 2-yl)-3-(o-tolyl)urea |
| 367 | 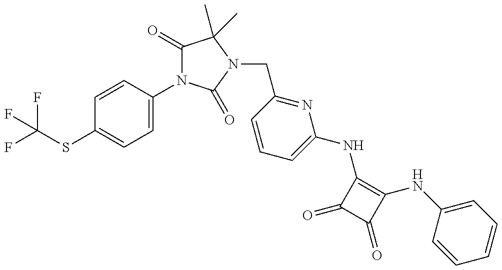 [SMILES: CC1(C)C(=O)N(c2ccc(SC(F)(F)F)cc2)C(=O)N1Cc1cccc(Nc2c(Nc3ccccc3)c(=O)c2=O)n1] | 1-((6-((3,4-dioxo-2- (phenylamino)cyclobut-1-en-1- yl)amino)pyridin-2-yl)methyl)- 5,5-dimethyl-3-(4- ((trifluoromethyl)thio)phenyl) imidazolidine-2,4-dione |
| 368 | 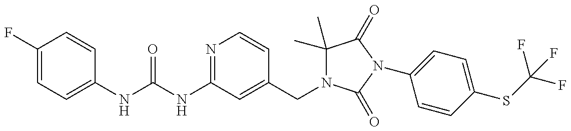 [SMILES: CC1(C)C(=O)N(c2ccc(SC(F)(F)F)cc2)C(=O)N1Cc1ccnc(NC(=O)Nc2ccc(F)cc2)c1] | 1-(4-((5,5-dimethyl-2,4-dioxo-3- (4- ((trifluoromethyl)thio)phenyl) imidazolidin-1-yl)methyl)pyridin-2- yl)-3-(4-fluorophenyl)urea |
| 369 | 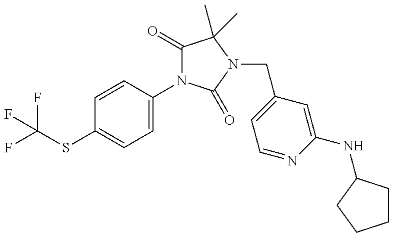 [SMILES: CC1(C)C(=O)N(c2ccc(SC(F)(F)F)cc2)C(=O)N1Cc1ccnc(NC2CCCC2)c1] | 1-((2-(cyclopentylamino)pyridin- 4-yl)methyl)-5,5-dimethyl-3-(4- ((trifluoromethyl)thio)phenyl) imidazolidine-2,4-dione |
| 370 | 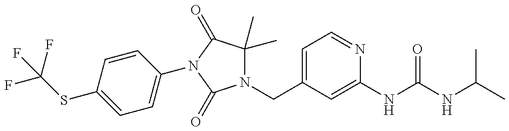 [SMILES: CC(C)NC(=O)Nc1cc(CN2C(=O)N(c3ccc(SC(F)(F)F)cc3)C(=O)C2(C)C)ccn1] | 1-(4-((5,5-dimethyl-2,4-dioxo-3- (4- ((trifluoromethyl)thio)phenyl) imidazolidin-1-yl)methyl)pyridin-2- yl)-3-isopropylurea |
| 371 | 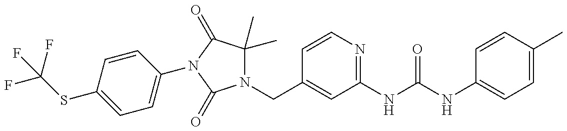 [SMILES: Cc1ccc(NC(=O)Nc2cc(CN3C(=O)N(c4ccc(SC(F)(F)F)cc4)C(=O)C3(C)C)ccn2)cc1] | 1-(4-((5,5-dimethyl-2,4-dioxo-3- (4- ((trifluoromethyl)thio)phenyl) imidazolidin-1-yl)methyl)pyridin-2- yl)-3-(p-tolyl)urea |
| 372 | 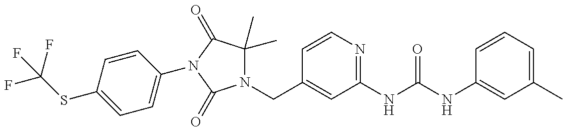 [SMILES: Cc1cccc(NC(=O)Nc2cc(CN3C(=O)N(c4ccc(SC(F)(F)F)cc4)C(=O)C3(C)C)ccn2)c1] | 1-(4-((5,5-dimethyl-2,4-dioxo-3- (4- ((trifluoromethyl)thio)phenyl) imidazolidin-1-yl)methyl)pyridin-2- yl)-3-(m-tolyl)urea |
| 373 | 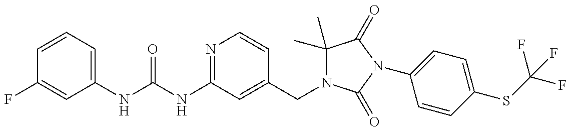 [SMILES: CC1(C)C(=O)N(c2ccc(SC(F)(F)F)cc2)C(=O)N1Cc1ccnc(NC(=O)Nc2cccc(F)c2)c1] | 1-(4-((5,5-dimethyl-2,4-dioxo-3- (4- ((trifluoromethyl)thio)phenyl) imidazolidin-1-yl)methyl)pyridin-2- yl)-3-(3-fluorophenyl)urea |
| 374 | 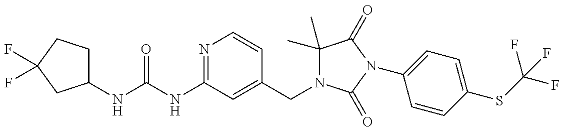 [SMILES: CC1(C)C(=O)N(c2ccc(SC(F)(F)F)cc2)C(=O)N1Cc1ccnc(NC(=O)NC2CCC(F)(F)C2)c1] | 1-(3,3-difluorocyclopenty)-3-(4- ((5,5-dimethyl-2,4-dioxo-3-(4- ((trifluoromethyl)thio)phenyl) imidazolidin-1-yl)methyl)pyridin-2- yl)urea |
| 375 | 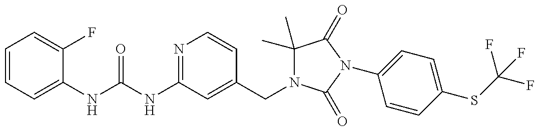 [SMILES: CC1(C)C(=O)N(c2ccc(SC(F)(F)F)cc2)C(=O)N1Cc1ccnc(NC(=O)Nc2ccccc2F)c1] | 1-(4-((5,5-dimethyl-2,4-dioxo-3- (4- ((trifluoromethyl)thio)phenyl) imidazolidin-1-yl)methyl)pyridin-2- yl)-3-(2-fluorophenyl)urea |
| 376 | 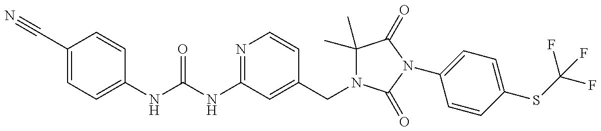 [SMILES: CC1(C)C(=O)N(c2ccc(SC(F)(F)F)cc2)C(=O)N1Cc1ccnc(NC(=O)Nc2ccc(C#N)cc2)c1] | 1-(4-cyanophenyl)-3-(4-((5,5- dimethyl-2,4-dioxo-3-(4- ((trifluoromethyl)thio)phenyl) imidazolidin-1-yl)methyl)pyridin-2- yl)urea |
| 377 | 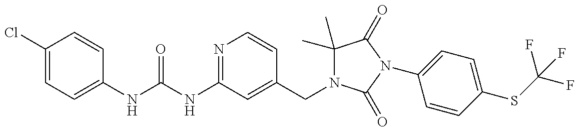 [SMILES: CC1(C)C(=O)N(c2ccc(SC(F)(F)F)cc2)C(=O)N1Cc1ccnc(NC(=O)Nc2ccc(Cl)cc2)c1] | 1-(4-chlorophenyl)-3-(4-((5,5- dimethyl-2,4-dioxo-3-(4- ((trifluoromethyl)thio)phenyl) imidazolidin-1-yl)methyl)pyridin-2- yl)urea |
| 378 | 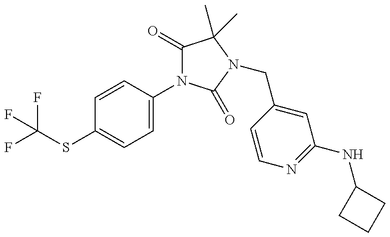 [SMILES: CC1(C)C(=O)N(c2ccc(SC(F)(F)F)cc2)C(=O)N1Cc1ccnc(NC2CCC2)c1] | 1-((2-(cyclobutylamino)pyridin- 4-yl)methyl)-5,5-dimethyl-3-(4- ((trifluoromethyl)thio)phenyl) imidazolidine-2,4-dione |
| 379 | 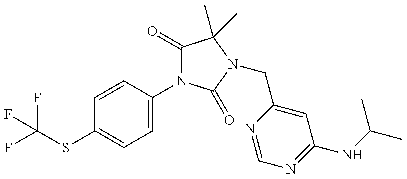 [SMILES: CC(C)Nc1cc(CN2C(=O)N(c3ccc(SC(F)(F)F)cc3)C(=O)C2(C)C)ncn1] | 1-((6- (isopropylamino)pyrimidin-4- yl)methyl)-5,5-dimethyl-3-(4- ((trifluoromethyl)thio)phenyl) imidazolidine-2,4-dione |
| 380 | 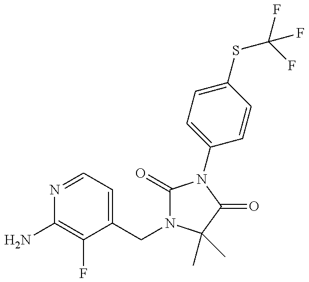 [SMILES: CC1(C)C(=O)N(c2ccc(SC(F)(F)F)cc2)C(=O)N1Cc1ccnc(N)c1F] | 1-((2-amino-3-fluoropyridin-4- yl)methyl)-5,5-dimethyl-3-(4- ((trifluoromethyl)thio)phenyl) imidazolidine-2,4-dione |
| 381 | 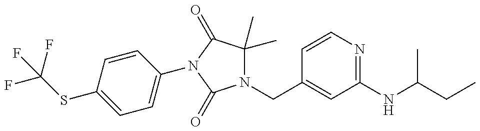 [SMILES: CCC(C)Nc1cc(CN2C(=O)N(c3ccc(SC(F)(F)F)cc3)C(=O)C2(C)C)ccn1] | 1-((2-(sec-butylamino)pyridin-4- yl)methyl)-5,5-dimethyl-3-(4- ((trifluoromethyl)thio)phenyl) imidazolidine-2,4-dione |
| 382 | 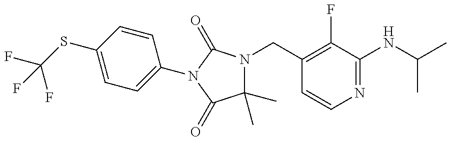 [SMILES: CC(C)Nc1nccc(CN2C(=O)N(c3ccc(SC(F)(F)F)cc3)C(=O)C2(C)C)c1F] | 1-((3-fluoro-2- (isopropylamino)pyridin-4- yl)methyl)-5,5-dimethyl-3-(4- ((trifluoromethyl)thio)phenyl) imidazolidine-2,4-dione |
| 383 | 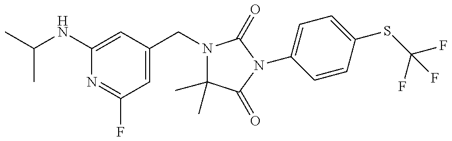 [SMILES: CC(C)Nc1cc(CN2C(=O)N(c3ccc(SC(F)(F)F)cc3)C(=O)C2(C)C)cc(F)n1] | 1-((2-fluoro-6- (isopropylamino)pyridin-4- yl)methyl)-5,5-dimethyl-3-(4- ((trifluoromethyl)thio)phenyl) imidazolidine-2,4-dione |
| 384 | 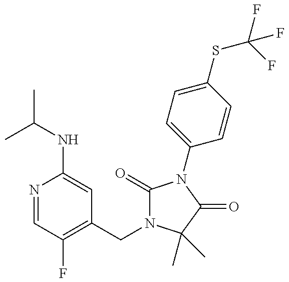 [SMILES: CC(C)Nc1cc(CN2C(=O)N(c3ccc(SC(F)(F)F)cc3)C(=O)C2(C)C)c(F)cn1] | 1-((5-fluoro-2- (isopropylamino)pyridin-4- yl)methyl)-5,5-dimethyl-3-(4- ((trifluoromethyl)thio)phenyl) imidazolidine-2,4-dione |
| 385 | 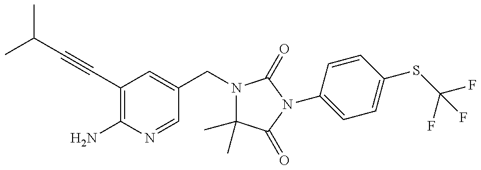 [SMILES: CC(C)C#Cc1cc(CN2C(=O)N(c3ccc(SC(F)(F)F)cc3)C(=O)C2(C)C)cnc1N] | 1-((6-amino-5-(3-methylbut-1- yn-1-yl)pyridin-3-yl)methyl)- 5,5-dimethyl-3-(4- ((trifluoromethyl)thio)phenyl) imidazolidine-2,4-dione |
| 386 | 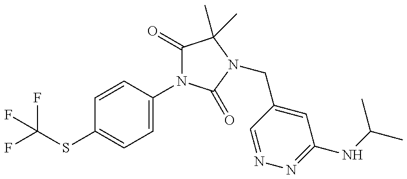 [SMILES: CC(C)Nc1cc(CN2C(=O)N(c3ccc(SC(F)(F)F)cc3)C(=O)C2(C)C)cnn1] | 1-((6-(isopropylamino)pyridazin- 4-yl)methyl)-5,5-dimethyl-3-(4- ((trifluoromethyl)thio)phenyl) imidazolidine-2,4-dione |
| 387 | 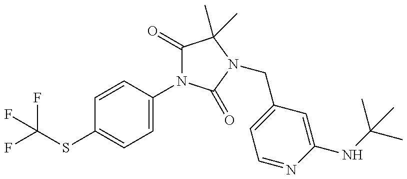 [SMILES: CC(C)(C)Nc1cc(CN2C(=O)N(c3ccc(SC(F)(F)F)cc3)C(=O)C2(C)C)ccn1] | 1-((2-(tert-butylamino)pyridin-4- yl)methyl)-5,5-dimethyl-3-(4- ((trifluoromethyl)thio)phenyl) imidazolidine-2,4-dione |
| 388 | 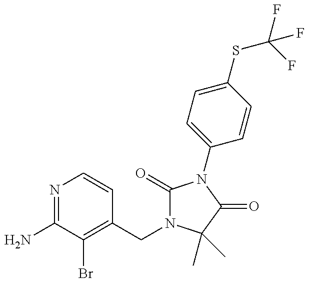 [SMILES: CC1(C)C(=O)N(c2ccc(SC(F)(F)F)cc2)C(=O)N1Cc1ccnc(N)c1Br] | 1-((2-amino-3-bromopyridin-4- yl)methyl)-5,5-dimethyl-3-(4- ((trifluoromethyl)thio)phenyl) imidazolidine-2,4-dione |
| 389 | 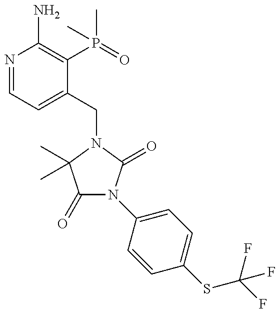 [SMILES: CC1(C)C(=O)N(c2ccc(SC(F)(F)F)cc2)C(=O)N1Cc1ccnc(N)c1P(C)(C)=O] | 1-((2-amino-3- (dimethylphosphoryl)pyridin-4- yl)methyl)-5,5-dimethyl-3-(4- ((trifluoromethyl)thio)phenyl) imidazolidine-2,4-dione |
| 390 | 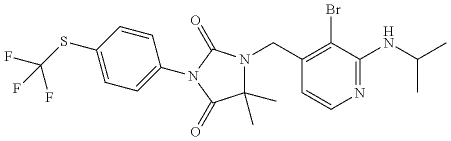 [SMILES: CC(C)Nc1nccc(CN2C(=O)N(c3ccc(SC(F)(F)F)cc3)C(=O)C2(C)C)c1Br] | 1-((3-bromo-2- (isopropylamino)pyridin-4- yl)methyl)-5,5-dimethyl-3-(4- ((trifluoromethyl)thio)phenyl) imidazolidine-2,4-dione |
| 391 | 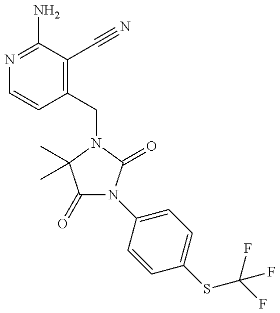 [SMILES: CC1(C)C(=O)N(c2ccc(SC(F)(F)F)cc2)C(=O)N1Cc1ccnc(N)c1C#N] | 2-amino-4-((5,5-dimethyl-2,4- dioxo-3-(4- ((trifluoromethyl)thio)phenyl) imidazolidin-1- yl)methyl)nicotinonitrile |
| 392 | 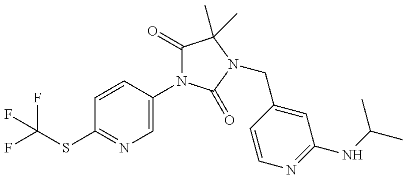 [SMILES: CC(C)Nc1cc(CN2C(=O)N(c3ccc(SC(F)(F)F)nc3)C(=O)C2(C)C)ccn1] | 1-((2-(isopropylamino)pyridin-4- yl)methyl)-5,5-dimethyl-3-(6- ((trifluoromethyl)thio)pyridin-3- yl)imidazolidine-2,4-dione |
| 393 | 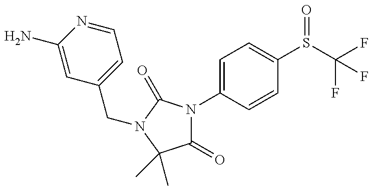 [SMILES: CC1(C)C(=O)N(c2ccc(S(=O)C(F)(F)F)cc2)C(=O)N1Cc1ccnc(N)c1] | 1-((2-aminopyridin-4- yl)methyl)-5,5-dimethyl-3-(4- ((trifluoromethyl)sulfinyl)phenyl) imidazolidine-2,4-dione |
| 394 | 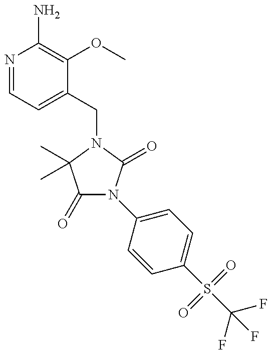 [SMILES: COc1c(CN2C(=O)N(c3ccc(S(=O)(=O)C(F)(F)F)cc3)C(=O)C2(C)C)ccnc1N] | 1-((2-amino-3-methoxypyridin- 4-yl)methyl)-5,5-dimethyl-3-(4- ((trifluoromethyl)sulfonyl)phenyl) imidazolidine-2,4-dione |
| 395 | 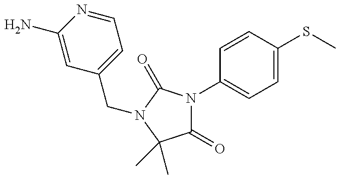 [SMILES: CSc1ccc(N2C(=O)N(Cc3ccnc(N)c3)C(C)(C)C2=O)cc1] | 1-((2-aminopyridin-4- yl)methyl)-5,5-dimethyl-3-(4- (methylthio)phenyl)imidazolidine- 2,4-dione |
| 396 | 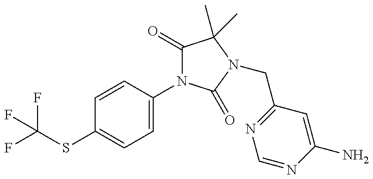 [SMILES: CC1(C)C(=O)N(c2ccc(SC(F)(F)F)cc2)C(=O)N1Cc1cc(N)ncn1] | 1-((6-aminopyrimidin-4- yl)methyl)-5,5-dimethyl-3-(4- ((trifluoromethyl)thio)phenyl) imidazolidine-2,4-dione |
| 397 | 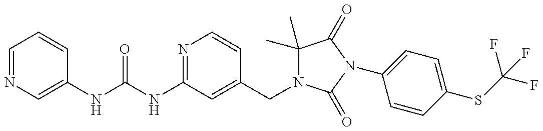 [SMILES: CC1(C)C(=O)N(c2ccc(SC(F)(F)F)cc2)C(=O)N1Cc1ccnc(NC(=O)Nc2cccnc2)c1] | 1-(4-((5,5-dimethyl-2,4-dioxo-3- (4- ((trifluoromethyl)thio)phenyl) imidazolidin-1-yl)methyl)pyridin-2- yl)-3-(pyridin-3-yl)urea |
| 398 | 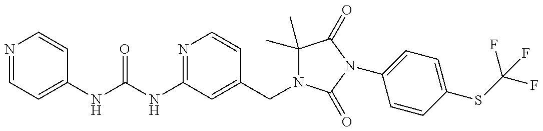 [SMILES: CC1(C)C(=O)N(c2ccc(SC(F)(F)F)cc2)C(=O)N1Cc1ccnc(NC(=O)Nc2ccncc2)c1] | 1-(4-((5,5-dimethyl-2,4-dioxo-3- (4- ((trifluoromethyl)thio)phenyl) imidazolidin-1-yl)methyl)pyridin-2- yl)-3-(pyridin-4-yl)urea |
| 399 | 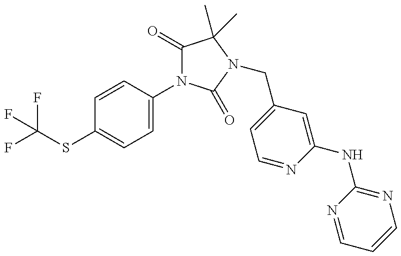 [SMILES: CC1(C)C(=O)N(c2ccc(SC(F)(F)F)cc2)C(=O)N1Cc1ccnc(Nc2ncccn2)c1] | 5,5-dimethyl-1-((2-(pyrimidin-2- ylamino)pyridin-4-yl)methyl)-3- (4- ((trifluoromethyl)thio)phenyl) imidazolidine-2,4-dione |
| 400 | 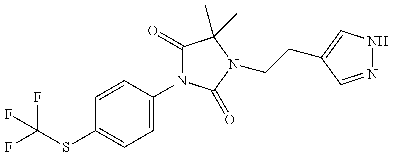 [SMILES: CC1(C)C(=O)N(c2ccc(SC(F)(F)F)cc2)C(=O)N1CCc1cn[nH]c1] | 1-(2-(1H-pyrazol-4-yl)ethyl)- 5,5-dimethyl-3-(4- ((trifluoromethyl)thio)phenyl) imidazolidine-2,4-dione |
| 401 | 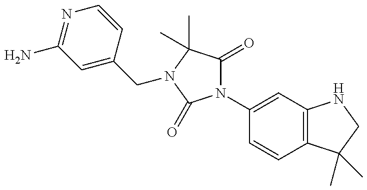 [SMILES: CC1(C)CNc2cc(N3C(=O)N(Cc4ccnc(N)c4)C(C)(C)C3=O)ccc21] | 1-((2-aminopyridin-4- yl)methyl)-3-(3,3- dimethylindolin-6-yl)-5,5- dimethylimidazolidine-2,4-dione |
| 402 | 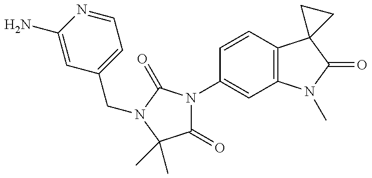 [SMILES: CN1C(=O)C2(CC2)c2ccc(N3C(=O)N(Cc4ccnc(N)c4)C(C)(C)C3=O)cc21] | 1-((2-aminopyridin-4- yl)methyl)-5,5-dimethyl-3-(1′- methyl-2′- oxospiro[cyclopropane-1,3′- indolin]-6′-yl)imidazolidine-2,4- dione |
| 403 | 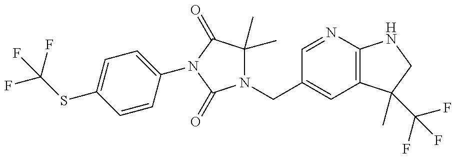 [SMILES: CC1(C)C(=O)N(c2ccc(SC(F)(F)F)cc2)C(=O)N1Cc1cnc2c(c1)C(C)(C(F)(F)F)CN2] | 5,5-dimethyl-1-((3-methyl-3- (trifluoromethyl)-2,3-dihydro- 1H-pyrrolo[2,3-b]pyridin-5- yl)methyl)-3-(4- ((trifluoromethyl)thio)phenyl) imidazolidine-2,4-dione |
| 405 | 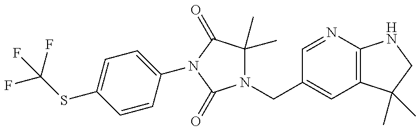 [SMILES: CC1(C)CNc2ncc(CN3C(=O)N(c4ccc(SC(F)(F)F)cc4)C(=O)C3(C)C)cc21] | 1-((3,3-dimethyl-2,3-dihydro- 1H-pyrrolo[2,3-b]pyridin-5- yl)methyl)-5,5-dimethyl-3-(4- ((trifluoromethyl)thio)phenyl) imidazolidine-2,4-dione |
| 406 | 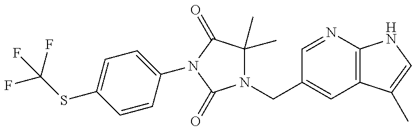 [SMILES: Cc1c[nH]c2ncc(CN3C(=O)N(c4ccc(SC(F)(F)F)cc4)C(=O)C3(C)C)cc12] | 5,5-dimethyl-1-((3-methyl-1H- pyrrolo[2,3-b]pyridin-5- yl)methyl)-3-(4- ((trifluoromethyl)thio)phenyl) imidazolidine-2,4-dione |
| 407 | 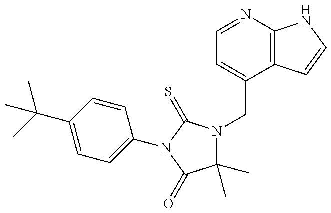 [SMILES: CC(C)(C)c1ccc(N2C(=O)C(C)(C)N(Cc3ccnc4[nH]ccc34)C2=S)cc1] | 1-((1H-pyrrolo[2,3-b]pyridin-4- yl)methyl)-3-(4-(tert- butyl)phenyl)-5,5-dimethyl-2- thioxoimidazolidin-4-one |
| 408 | 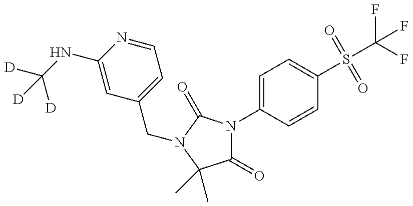 [SMILES: [2H]C([2H])([2H])Nc1cc(CN2C(=O)N(c3ccc(S(=O)(=O)C(F)(F)F)cc3)C(=O)C2(C)C)ccn1] | 5,5-dimethyl-1-((2-((methyl- d3)amino)pyridin-4-yl)methyl)- 3-(4- ((trifluoromethyl)sulfonyl)phenyl) imidazolidine-2,4-dione |

In some embodiments, the compound is a pharmaceutically acceptable salt of a compound described in Table 10.

Also provided herein is a compound selected from the Examples disclosed herein.

### Further Forms of Compounds

The compounds disclosed herein can exist as therapeutically acceptable salts. The present invention includes compounds listed above in the form of salts, including acid addition salts. Suitable salts include those formed with both organic and inorganic acids. Such acid addition salts will normally be pharmaceutically acceptable. However, salts of non-pharmaceutically acceptable salts may be of utility in the preparation and purification of the compound in question. Basic addition salts may also be formed and be pharmaceutically acceptable. For a more complete discussion of the preparation and selection of salts, refer to Pharmaceutical Salts: Properties, Selection, and Use (Stahl, P. Heinrich. Wiley-VCHA, Zurich, Switzerland, 2002).

The term “therapeutically acceptable salt,” as used herein, represents salts or zwitterionic forms of the compounds disclosed herein which are water or oil-soluble or dispersible and therapeutically acceptable as defined herein. The salts can be prepared during the final isolation and purification of the compounds or separately by reacting the appropriate compound in the form of the free base with a suitable acid. Representative acid addition salts include acetate, adipate, alginate. L-ascorbate, aspartate, benzoate, benzenesulfonate (besylate), bisulfate, butyrate, camphorate, camphorsulfonate, citrate, digluconate, formate, fumarate, gentisate, glutarate, glycerophosphate, glycolate, hemisulfate, heptanoate, hexanoate, hippurate, hydrochloride, hydrobromide, hydroiodide, 2-hydroxyethansulfonate (isethionate), lactate, maleate, malonate, DL-mandelate, mesitylenesulfonate, methanesulfonate, naphthylenesulfonate, nicotinate, 2-naphthalenesulfonate, oxalate, pamoate, pectinate, persulfate, 3-phenylproprionate, phosphonate, picrate, pivalate, propionate, pyroglutamate, succinate, sulfonate, tartrate, L-tartrate, trichloroacetate, trifluoroacetate, phosphate, glutamate, bicarbonate, para-toluenesulfonate (p-tosylate), and undecanoate. Also, basic groups in the compounds disclosed herein can be quaternized with methyl, ethyl, propyl, and butyl chlorides, bromides, and iodides; dimethyl, diethyl, dibutyl, and diamyl sulfates; decyl, lauryl, myristyl, and steryl chlorides, bromides, and iodides; and benzyl and phenethyl bromides. Examples of acids which can be employed to form therapeutically acceptable addition salts include inorganic acids such as hydrochloric, hydrobromic, sulfuric, and phosphoric, and organic acids such as oxalic, maleic, succinic, and citric. Salts can also be formed by coordination of the compounds with an alkali metal or alkaline earth ion. Hence, the present invention contemplates sodium, potassium, magnesium, and calcium salts of the compounds disclosed herein, and the like.

Basic addition salts can be prepared during the final isolation and purification of the compounds by reacting a carboxy group with a suitable base such as the hydroxide, carbonate, or bicarbonate of a metal cation or with ammonia or an organic primary, secondary, or tertiary amine. The cations of therapeutically acceptable salts include lithium, sodium, potassium, calcium, magnesium, and aluminum, as well as nontoxic quaternary amine cations such as ammonium, tetramethylammonium, tetraethylammonium, methylamine, dimethylamine, trimethylamine, triethylamine, diethylamine, ethylamine, tributylamine, pyridine, N,N-dimethylaniline, N-methylpiperidine, N-methylmorpholine, dicyclohexylamine, procaine, dibenzylamine. N,N-dibenzylphenethylamine, 1-ephenamine, and ‘,N′-dibenzylethylenediamine. Other representative organic amines useful for the formation of base addition salts include ethylenediamine, ethanolamine, diethanolamine, piperidine, and piperazine.

A salt of a compound can be made by reacting the appropriate compound in the form of the free base with the appropriate acid.

The te“m” prod“ug” refers to a compound that is made more active in vivo. Certain compounds disclosed herein may also exist as prodrugs, as described in Hydrolysis in Drug and Prodrug Metabolism: Chemistry. Biochemistry, and Enzymology (Testa, Bernard and Mayer, Joachim M. Wiley-VHCA, Zurich, Switzerland 2003). Prodrugs of the compounds described herein are structurally modified forms of the compound that readily undergo chemical changes under physiological conditions to provide the compound. Additionally, prodrugs can be converted to the compound by chemical or biochemical methods in an ex vivo environment.

### Definitions

As used herein, the terms below have the meanings indicated.

When ranges of values are disclosed, and the notation “from n₁ . . . to n₂” or “between n₁ . . . and n₂” is used, where n₁ and n₂ are the numbers, then unless otherwise specified, this notation is intended to include the numbers themselves and the range between them. This range may be integral or continuous between and including the end values. By way of example, the range “from 2 to 6 carbons” is intended to include two, three, four, five, and six carbons, since carbons come in integer units. Compare, by way of example, the range “from 1 to 3 μM (micromolar),” which is intended to include 1 μM, 3 μM, and everything in between to any number of significant figures (e.g., 1.255 μM, 2.1 μM, 2.9999 μM, etc.).

The term “about,” as used herein, is intended to qualify the numerical values which it modifies, denoting such a value as variable within a range. When no particular range, such as a margin of error or a standard deviation to a mean value given in a chart or table of data, is recited, the term “about” should be understood to mean the greater of the range which would encompass the recited value and the range which would be included by rounding up or down to that figure as well, taking into account significant figures, and the range which would encompass the recited value plus or minus 20%.

The term “acyl,” as used herein refers to the group —C(═O)—R, where R is alkyl, alkenyl, alkynyl, aryl, cycloalkyl, heteroaryl, heterocycle, or any other moiety were the atom attached to the carbonyl is carbon. An “acetyl” group refers to a —C(═O)CH₃ group.

The term “alkenyl,” as used herein refers to a straight-chain or branched-chain hydrocarbon radical having one or more double bonds and containing from 2 to 20 carbon atoms. In certain embodiments, alkenyl includes 2 to 6 carbon atoms. The term “alkenylene” refers to a divalent alkenyl. In some embodiments, an alkenyl is selected from ethenyl (i.e., vinyl), propenyl (i.e., allyl), butenyl, pentenyl, pentadienyl, and the like. Non-limiting examples of an alkenyl group include —CH═CH₂, —C(CH₃)═CH₂, —CH═CHCH₃, —C(CH₃)═CHCH₃, and —CH₂CH═CH₂.

The term “alkoxy” refers to a (alkyl)-O— group, where alkyl is as defined herein. In some embodiments, the alkoxy group is a C₁-C₆alkoxy, which refers to a (C₁-C₆alkyl)-O-group. Examples of alkyl groups include methoxy, ethoxy, n-propoxy, isopropoxy, n-butoxy, iso-butoxy, sec-butoxy, tert-butoxy, and the like.

An “alkyl” group refers to an aliphatic hydrocarbon group. In some embodiments, the alkyl is a straight-chain or branched-chain aliphatic hydrocarbon group containing from 1 to 20 carbon atoms. In certain embodiments, alkyl includes 1 to 10 carbon atoms. In further embodiments, the alkyl includes 1 to 8 carbon atoms. Examples of alkyl radicals include methyl, ethyl, n-propyl, isopropyl, n-butyl, isobutyl, sec-butyl, tert-butyl, pentyl, iso-amyl, hexyl, octyl, nonyl and the like. In some embodiments, an alkyl is a C₁-C₆alkyl. In one aspect the alkyl is methyl, ethyl, propyl, iso-propyl, n-butyl, iso-butyl, sec-butyl, or t-butyl. The term “alkylene” refers to a divalent alkyl, such as methylene (—CH₂—). In some embodiments, an alkylene is a C₁-C₆alkylene. In other embodiments, an alkylene is a C₁-C₄alkylene. Typical alkylene groups include, but are not limited to, —CH₂—, —CH₂CH₂—, —CH₂CH₂CH₂—, —CH₂CH₂CH₂CH₂—, and the like.

The term “amino,” as used herein refers to —NRR′, wherein R and R′ are independently selected from hydrogen, alkyl, acyl, heteroalkyl, aryl, cycloalkyl, heteroaryl, and heterocycloalkyl, any of which may themselves be optionally substituted. Additionally. R and R′ may combine to form heterocycloalkyl, either of which may be optionally substituted. In one aspect, “amino” as used herein refers to an —NH₂ group.

The term “alkylthio,” as used herein refers to an alkyl thioether (R—S—) radical wherein the term alkyl is as defined above and wherein the sulfur may be singly or doubly oxidized. Examples of suitable alkyl thioether radicals include methylthio, ethylthio, n-propylthio, isopropylthio, n-butylthio, iso-butylthio, sec-butylthio, tert-butylthio, methanesulfonyl, ethanesulfinyl, and the like. In some embodiments, the term “alkylthio” refers to the —S-(alkyl) group. In some embodiments, the alkylthio group is a C₁-C₆alkylthio, which refers to the —S—(C₁-C₆alkyl) group.

The term “alkynyl,” as used herein refers to a straight-chain or branched chain hydrocarbon radical having one or more triple bonds and containing from 2 to 20 carbon atoms. In certain embodiments, said alkynyl comprises from 2 to 6 carbon atoms. In further embodiments, said alkynyl comprises from 2 to 4 carbon atoms. In one embodiment, an alkenyl group has the formula —C≡C—R, wherein R refers to the remaining portions of the alkynyl group. In some embodiments, R is H or an alkyl. In some embodiments, an alkynyl is selected from ethynyl, propynyl, butynyl, pentynyl, hexynyl, and the like. Non-limiting examples of an alkynyl group include —C≡CH, —C≡CCH₃—C≡CCH₂CH₃, —CH₂C≡CH. The term “alkynylene” refers to a carbon-carbon triple bond attached at two positions such as ethynylene (—C≡C—). Examples of alkynyl radicals include ethynyl, propynyl, hydroxypropynyl, butyn-1-yl, butyn-2-yl, pentyn-1-yl, 3-methylbutyn-1-yl, hexyn-2-yl, and the like. Unless otherwise specified, the term “alkynyl” may include “alkynylene” groups.

The term “aromatic” refers to a planar ring having a delocalized n-electron system containing 4n+2 π electrons, where n is an integer. The term “aromatic” includes both carbocyclic aryl (“aryl”, e.g., phenyl) and heterocyclic aryl (or “heteroaryl” or “heteroaromatic”) groups (e.g., pyridine). The term includes monocyclic or fused-ring polycyclic (i.e., rings which share adjacent pairs of carbon atoms) groups.

The term “carbocyclic” or “carbocycle” refers to a ring or ring system where the atoms forming the backbone of the ring are all carbon atoms. The term thus distinguishes carbocyclic from “heterocyclic” rings or “heterocycles” in which the ring backbone contains at least one atom which is different from carbon. In some embodiments, at least one of the two rings of a bicyclic carbocycle is aromatic. In some embodiments, both rings of a bicyclic carbocycle are aromatic. Carbocycles include aryls and cycloalkyls.

The term “aryl” as used herein means a carbocyclic aromatic system containing one, two or three rings wherein such polycyclic ring systems are fused together. The te“rm” a“yl” embraces aromatic groups such as phenyl, naphthyl, anthracenyl, and phenanthryl. In one aspect, aryl is phenyl or a naphthyl. In some embodiments, an aryl is a phenyl. In some embodiments, an aryl is a phenyl, naphthyl, indanyl, indenyl, or tetrahydronaphthyl. In some embodiments, an aryl is a C₆-C₁₀aryl. Depending on the structure, an aryl group is a monoradical or a diradical (i.e., an arylene group).

The term “aryloxy” as used herein refers to an aryl group attached to the parent molecular moiety through an oxy. In some embodiments, aryloxy as used herein refers to the aryl-O— group. In some embodiments, aryloxy is phenoxy, or a phenyl-O— group

The terms “benzo” and “benz,” as used herein refer to fused bicyclic or polycyclic ring system that is formed with benzene as one of the rings. Examples include benzofuran, benzothiophene, and benzimidazole.

The term “cycloalkyl,” as used herein refers to a saturated or partially saturated monocyclic, bicyclic or tricyclic alkyl group wherein each cyclic moiety contains from 3 to 12 carbon atom ring members and which may optionally be a benzo fused ring system which is optionally substituted as defined herein. In some embodiments, cycloalkyl groups include groups having from 3 to 10 ring atoms. In certain embodiments, said cycloalkyl will comprise from 5 to 7 carbon atoms. In certain embodiments, said cycloalkyl will comprise from 3 to 6 carbon atoms. Examples of such cycloalkyl groups include cyclopropyl, cyclobutyl, cyclopentyl, cyclohexyl, cycloheptyl, tetrahydronaphthyl, indanyl, octahydronaphthyl, 2,3-dihydro-1H-indenyl, adamantyl and the like. “Bicyclic” and “tricyclic” as used herein are intended to include both fused ring systems, such as decahydronaphthalene, octahydronaphthalene as well as the multicyclic (multicentered) saturated or partially unsaturated type. The latter type of isomer is exemplified in general by, bicyclo[1,1,1]pentane, camphor, adamantane, and bicyclo[3,2,1]octane. In some embodiments, a cycloalkyl is a C₃-C₆cycloalkyl. In some embodiments, a cycloalkyl is a C₃-C₄cycloalkyl.

The term “heterocycle” or “heterocyclic” refers to heteroaromatic rings (also known as heteroaryls) and heterocycloalkyl rings containing one to four heteroatoms in the ring(s), where each heteroatom in the ring(s) is selected from O, S and N, wherein each heterocyclic group has from 3 to 10 atoms in its ring system, and with the proviso that any ring does not contain two adjacent O or S atoms. Non-aromatic heterocyclic groups (also known as heterocycloalkyls) include rings having 3 to 10 atoms in its ring system and aromatic heterocyclic groups include rings having 5 to 10 atoms in its ring system. The heterocyclic groups include benzo-fused ring systems. Examples of non-aromatic heterocyclic groups are pyrrolidinyl, tetrahydrofuranyl, dihydrofuranyl, tetrahydrothienyl, oxazolidinonyl, tetrahydropyranyl, dihydropyranyl, tetrahydrothiopyranyl, piperidinyl, morpholinyl, thiomorpholinyl, thioxanyl, piperazinyl, aziridinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, pyrrolin-2-yl, pyrrolin-3-yl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl, indolin-2-onyl, isoindolin-1-onyl, isoindoline-1,3-dionyl, 3,4-dihydroisoquinolin-1(2H)-onyl, 3,4-dihydroquinolin-2(1H)-onyl, isoindoline-1,3-dithionyl, benzo[d]oxazol-2(3H)-onyl, 1H-benzo[d]imidazol-2(3H)-onyl, benzo[d]thiazol-2(3H)-onyl, and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups are either C-attached (or C-linked) or N-attached where such is possible. For instance, a group derived from pyrrole includes both pyrrol-1-yl (N-attached) or pyrrol-3-yl (C-attached). Further, a group derived from imidazole includes imidazol-1-yl or imidazol-3-yl (both N-attached) or imidazol-2-yl, imidazol-4-yl or imidazol-5-yl (all C-attached). The heterocyclic groups include benzo-fused ring systems. Non-aromatic heterocycles are optionally substituted with one or two oxo (═O) moieties, such as pyrrolidin-2-one. In some embodiments, at least one of the two rings of a bicyclic heterocycle is aromatic. In some embodiments, both rings of a bicyclic heterocycle are aromatic.

The terms “heteroaryl” or, alternatively, “heteroaromatic” refers to an aryl group that includes one or more ring heteroatoms selected from nitrogen, oxygen and sulfur. In some embodiments, the te“m “heteroar“l,” as used herein refers to a 3 to 15 membered unsaturated heteromonocyclic ring, or a fused monocyclic, bicyclic, or tricyclic ring system in which at least one of the fused rings is aromatic, which contains at least one atom selected from N. O, and S. In certain embodiments, said heteroaryl will comprise from 1 to 4 heteroatoms as ring members. In further embodiments, said heteroaryl will comprise from 1 to 2 heteroatoms as ring members. In certain embodiments, said heteroaryl will comprise from 5 to 7 atoms. The term also embraces fused polycyclic groups wherein heterocyclic rings are fused with aryl rings, wherein heteroaryl rings are fused with other heteroaryl rings, wherein heteroaryl rings are fused with heterocycloalkyl rings, or wherein heteroaryl rings are fused with cycloalkyl rings. Examples of heteroaryl groups include pyrrolyl, imidazolyl, pyrazolyl, pyridyl, pyrimidinyl, pyrazinyl, pyridazinyl, triazinyl, triazolyl, furyl, thienyl, oxazolyl, isoxazolyl, oxadiazolyl, thiazolyl, thiadiazolyl, isothiazolyl, indolyl, isoindolyl, indolizinyl, benzimidazolyl, quinolyl, isoquinolyl, quinoxalinyl, quinazolinyl, indazolyl, benzotriazolyl, benzodioxolyl, benzopyranyl, benzoxazolyl, benzoxadiazolyl, benzothiazolyl, benzothiadiazolyl, benzofuranyl, benzothienyl, chromonyl, coumarinyl, benzopyranyl, tetrahydroquinolinyl, tetrazolopyridazinyl, tetrahydroisoquinolinyl, thienopyridinyl, furopyridinyl, pyrrolopyridinyl and the like. Exemplary tricyclic heterocyclic groups include carbazolyl, benzidolyl, phenanthrolinyl, dibenzofuranyl, acridinyl, phenanthridinyl, xanthenyl and the like. In some embodiments, a heteroaryl contains 0-4 N atoms in the ring. In some embodiments, a heteroaryl contains 1-4 N atoms in the ring. In some embodiments, a heteroaryl contains 0-4 N atoms, 0-1 O atoms, and 0-1 S atoms in the ring. In some embodiments, a heteroaryl contains 1-4 N atoms, 0-1 O atoms, and 0-1 S atoms in the ring. In some embodiments, heteroaryl is a C₁-C₉heteroaryl. In some embodiments, monocyclic heteroaryl is a C₁-C₅heteroaryl. In some embodiments, monocyclic heteroaryl is a 5-membered or 6-membered heteroaryl. In some embodiments, bicyclic heteroaryl is a C₆-C₉heteroaryl.

A “heterocycloalkyl” group refers to a cycloalkyl group that includes at least one heteroatom selected from nitrogen, oxygen and sulfur. In some embodiments, the term “heterocycloalkyl” as used herein each refer to a saturated, partially unsaturated, or fully unsaturated (but nonaromatic) monocyclic, bicyclic, or tricyclic heterocyclic group containing at least one heteroatom as a ring member, wherein each said heteroatom may be independently selected from nitrogen, oxygen, and sulfur. In certain embodiments, said heterocycloalkyl will comprise from 1 to 4 heteroatoms as ring members. In further embodiments, said heterocycloalkyl will comprise from 1 to 2 heteroatoms as ring members. In certain embodiments, said heterocycloalkyl will comprise from 3 to 8 ring members in each ring. In further embodiments, said heterocycloalkyl will comprise from 3 to 7 ring members in each ring. In yet further embodiments, said heterocycloalkyl will comprise from 5 to 6 ring members in each ring. “Heterocycloalkyl” and “heterocycle” are intended to include sulfones, sulfoxides, N-oxides of tertiary nitrogen ring members, and carbocyclic fused and benzo fused ring systems; additionally, both terms also include systems where a heterocycle ring is fused to an aryl group, as defined herein, or an additional heterocycle group. Examples of heterocycle groups include aziridinyl, azetidinyl, 1,3-benzodioxolyl, dihydroisoindolyl, dihydroisoquinolinyl, dihydrocinnolinyl, dihydrobenzodioxinyl, dihydro[1.3]oxazolo[4,5-b]pyridinyl, benzothiazolyl, dihydroindolyl, dihydropyridinyl, 1,3-dioxanyl, 1,4-dioxanyl, 1,3-dioxolanyl, isoindolinyl, morpholinyl, piperazinyl, pyrrolidinyl, tetrahydropyridinyl, piperidinyl, thiomorpholinyl, and the like. The heterocycle groups may be optionally substituted unless specifically prohibited. In one aspect, a heterocycloalkyl is a C₂-C₁₀heterocycloalkyl. In another aspect, a heterocycloalkyl is a C₄-C₁₀heterocycloalkyl. In some embodiments, a heterocycloalkyl is monocyclic or bicyclic. In some embodiments, a heterocycloalkyl is monocyclic and is a 3, 4, 5, 6, 7, or 8-membered ring. In some embodiments, a heterocycloalkyl is monocyclic and is a 3, 4, 5, or 6-membered ring. In some embodiments, a heterocycloalkyl is monocyclic and is a 3 or 4-membered ring. In some embodiments, a heterocycloalkyl contains 0-2 N atoms in the ring. In some embodiments, a heterocycloalkyl contains 0-2 N atoms, 0-2 O atoms and 0-1 S atoms in the ring.

The term “carbamate,” as used herein refers to an ester of carbamic acid (—NHCOO—) which may be attached to the parent molecular moiety from either the nitrogen or acid end, and which may be optionally substituted as defined herein.

The term “carboxyl” or “carboxy,” as used herein, refers to —C(═O)OH or the corresponding “carboxylate” anion, such as is in a carboxylic acid salt.

The term “cyano,” as used herein refers to —CN.

The term “halo,” or “halogen,” as used herein refers to fluorine, chlorine, bromine, or iodine. In some embodiments, halo is fluoro, chloro, or bromo.

The term “haloalkyl,” as used herein refers to an alkyl radical having the meaning as defined above wherein one or more hydrogens are replaced with a halogen. Specifically embraced are monohaloalkyl, dihaloalkyl and polyhaloalkyl radicals. A monohaloalkyl radical, for one example, may have an iodo, bromo, chloro or fluoro atom within the radical Dihalo and polyhaloalkyl radicals may have two or more of the same halo atoms or a combination of different halo radicals. Examples of haloalkyl radicals include fluoromethyl, difluoromethyl, trifluoromethyl, chloromethyl, dichloromethyl, trichloromethyl, pentafluoroethyl, heptafluoropropyl, difluorochloromethyl, dichlorofluoromethyl, difluoroethyl, difluoropropyl, dichloroethyl and dichloropropyl. “Haloalkylene” refers to a haloalkyl group attached at two or more positions. Examples include fluoromethylene (—CFH—), difluoromethylene (—CF₂—), chloromethylene (—CHCl—) and the like. In one aspect, a haloalkyl is a C₁-C₆haloalkyl. In another aspect, a haloalkyl is a C₁-C₄haloalkyl.

The term “haloalkoxy,” as used herein refers to a haloalkyl group attached to the parent molecular moiety through an oxygen atom. In one aspect, the haloalkoxy is a C₁-C₆haloalkoxy, which refers to a (C₁-C₆haloalkyl)-O— group. In another aspect, the haloalkoxy is a C₁-C₆haloalkoxy, which refers to a (C₁-C₄haloalkyl)-O— group.

The term “heteroalkyl” refers to an alkyl wherein 1 or more carbon atoms are replaced with a heteroatom. In some embodiments, “heteroalkyl” refers to an alkyl wherein 1 or more carbon atoms are replaced with one or more heteroatoms that are independently selected from NH, —N(alkyl), O, S, S(═O) and S(═O)₂. The attachment of the heteroatom(s) to the remainder of the compound is at a carbon atoms of the heteroalkyl. In some embodiments, up to two heteroatoms may be consecutive, such as, for example, —CH₂—NH—OCH₃. In some embodiments. “heteroalkyl” is an “alkoxyalkyl”, “alkylthioalkyl”, or “alkylaminoalkyl”. “Alkoxyalkyl” refers to an alkyl in which one hydrogen atom is replaced by an alkoxy group, as defined herein. In some embodiments, an alkoxyalkyl is a (C₁-C₆alkoxy)-C₁-C₆alkyl. Typical alkoxyalkyl groups include, but are not limited to, —CH₂OCH₃, —CH₂CH₂OCH₃, —CH₂CH₂CH₂OCH₃, —CH₂CH₂CH₂CH₂OCH₃, —CH₂OCH₂CH₃, —CH₂CH₂OCH₂CH₃, —CH₂CH₂CH₂OCH₂CH₃, —CH₂CH₂CH₂CH₂OCH₂CH₃, and the like. “Alkylthioalkyl” refers to an alkyl in which one hydrogen atom is replaced by an alkylthio group, as defined herein. In some embodiments, an alkoxyalkyl is a (C₁-C₆ alkylthio)-C₁-C₆alkyl. Typical alkoxyalkyl groups include, but are not limited to, —CH₂SCH₃, —CH₂CH₂SCH₃, —CH₂CH₂CH₂SCH₃, —CH₂CH₂CH₂CH₂SCH₃, —CH₂SCH₂CH₃, —CH₂CH₂SCH₂CH₃, —CH₂CH₂CH₂SCH₂CH₃, —CH₂CH₂CH₂CH₂SCH₂CH₃, and the like. “Alkylaminoalkyl” refers to an alkyl in which one hydrogen atom is replaced by an alkylamino group, as defined herein. In some embodiments, an alkoxyalkyl is a (C₁-C₆alkylamino)-C₁-C₆alkyl. Typical alkoxyalkyl groups include, but are not limited to, —CH₂NHCH₃, —CH₂CH₂NHCH₃, —CH₂CH₂CH₂NHCH₃, —CH₂CH₂CH₂CH₂NHCH₃, —CH₂NHCH₂CH₃, —CH₂CH₂NHCH₂CH₃, —CH₂CH₂CH₂NHCH₂CH₃, —CH₂CH₂CH₂CH₂NHCH₂CH₃, and the like.

The term “hydroxy,” or “hydroxyl,” as used herein refers to —OH.

The term “hydroxyalkyl,” as used herein refers to a hydroxy group attached to the parent molecular moiety through an alkyl group. In some embodiments, a hydroxyalkyl is a C₁-C₄hydroxyalkyl. Typical hydroxyalkyl groups include, but are not limited to, —CH₂OH, —CH₂CH₂OH, —CH₂CH₂CH₂OH, —CH₂CH₂CH₂CH₂OH, and the like.

The term “isocyanato” refers to a —NCO group.

The term “isothiocyanato” refers to a —NCS group.

The phrase “linear chain of atoms” refers to the longest straight chain of atoms independently selected from carbon, nitrogen, oxygen and sulfur.

The term “nitro.” as used herein refers to —NO₂.

The term “oxo,” as used herein refers to ═O.

The term “sulfanyl,” as used herein refers to —S—.

The term “sulfinyl,” as used herein refers to —S(═O)—.

The term “sulfonyl,” as used herein refers to a —S(═O)₂—, —S(═O)₂R, or —S(═O)₂R-group, with R as defined herein.

Any definition herein may be used in combination with any other definition to describe a composite structural group. By convention, the trailing element of any such definition is that which attaches to the parent moiety. For example, the composite group alkylamido would represent an alkyl group attached to the parent molecule through an amido group, and the term alkoxyalkyl would represent an alkoxy group attached to the parent molecule through an alkyl group.

When an outer variable of a parent moiety in this disclosure is defined by a chemical group that is preceded by a single, double or triple bond, the bond preceding this chemical group is attached to the parent moiety so long as the substitution results in a stable compound. By way of example, if a variable —Y of compound P—Y is defined as substituent —CH₂—C(═O)NH₂, then this substituent would be attached to the parent moiety (P) through the single bond on the methylene group on the left side of this chemical group.

When a variable of this disclosure is divalent which may include substituents preceded and followed by single, double, or triple bonds, or combinations thereof, then this divalent substituent can be orientated either from left to right or from right to left at the variable position so long as the substitution results in a stable compound. By way of example, when Y² of Formula (I) of this disclosure is —O—CH₂—, then Formula (I) can be either of the following two structures:

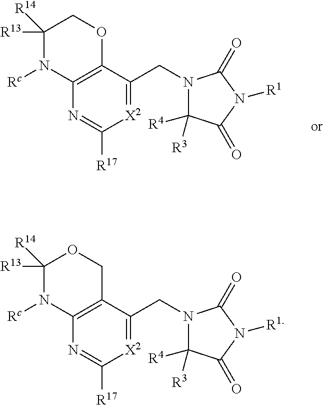

When a group is defined to be “null,” what is meant is that said group is absent.

In some embodiments, the term “optionally substituted” or “substituted” means that the referenced group is optionally substituted with one or more additional group(s) individually and independently selected from halogen. —CN, —NH₂. —NH(alkyl), —N(alkyl)₂, —OH, —CO₂H, —CO₂alkyl, —C(═O)NH₂, —C(═O)NH(alkyl), —C(═O)N(alkyl)₂, —S(═O)₂NH₂, —S(═O)₂NH(alkyl), —S(═O)₂N(alkyl)₂, alkyl, cycloalkyl, fluoroalkyl, heteroalkyl, alkoxy, fluoroalkoxy, heterocycloalkyl, aryl, heteroaryl, aryloxy, alkylthio, arylthio, alkylsulfoxide, arylsulfoxide, alkylsulfone, and arylsulfone. In some other embodiments, optional substituents are independently selected from halogen, —CN, —NH₂, —NH(CH₃), —N(CH₃)₂, —OH, —CO₂H, —CO₂(C₁-C₄alkyl), —C(═O)NH₂, —C(═O)NH(C₁-C₄alkyl), —C(═O)N(C₁-C₄alkyl)₂, —S(═O)₂NH₂, —S(═O)₂NH(C₁-C₄alkyl), —S(═O)₂N(C₁-C₄alkyl)₂, C₁-C₄alkyl, C₃-C₆cycloalkyl, C₁-C₄fluoroalkyl, C₁-C₄heteroalkyl, C₁-C₄alkoxy, C₁-C₄fluoroalkoxy. —SC₁-C₄alkyl, —S(═O)C₁-C₄alkyl, and —S(═O)₂C₁-C₄alkyl. In some embodiments, optional substituents are independently selected from halogen, —CN, —NH₂. —OH, —NH(CH₃), —N(CH₃)₂. —CH₃, —CH₂CH₃, —CHF₂, —CF₃, —OCH₃, —OCHF₂, and —OCF₃. In some embodiments, the “optionally substituted” or “substituted groups” defined herein are alkyl or heterocycloalkyl groups further substituted with one or two groups independently selected from halogen, —CN, —NH₂, —OH, —NH(CH₃), —N(CH₃)₂, —CH₃, —CH₂CH₃, —CHF₂, —CF₃, —OCH₃, —OCHF₂, and —OCF₃. In some embodiments, an optional substituent on an aliphatic carbon atom (acyclic or cyclic) includes oxo (═O).

Asymmetric centers exist in the compounds disclosed herein. These centers are designated by the symbols “R” or “S,” depending on the configuration of substituents around the chiral carbon atom. It should be understood that the invention encompasses all stereochemical isomeric forms, including diastereomeric, enantiomeric, and epimeric forms, as well as d-isomers and l-isomers, and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds disclosed herein may exist as geometric isomers. The present invention includes all cis, trans, syn, anti, entgegen (E), and zusammen (Z) isomers as well as the appropriate mixtures thereof. Additionally, compounds may exist as tautomers; all tautomeric isomers are provided by this invention.

Additionally, the compounds disclosed herein can exist in unsolvated as well as solvated forms with pharmaceutically acceptable solvents such as water, ethanol, and the like. In general, the solvated forms are considered equivalent to the unsolvated forms.

The term “bond” refers to a covalent linkage between two atoms, or two moieties when the atoms joined by the bond are considered to be part of larger substructure. A bond may be single, double, or triple unless otherwise specified. A dashed line between two atoms in a drawing of a molecule indicates that an additional bond may be present or absent at that position.

In some cases, the scope of the present disclosure includes pharmaceutically acceptable isotopically-labelled compounds of the compounds disclosed herein, wherein one or more atoms are replaced by atoms having the same atomic number, but an atomic mass or mass number different from the atomic mass or mass number usually found in nature. Examples of isotopes suitable for inclusion in the compounds disclosed herein include isotopes of hydrogen, such as ²H and ³H, carbon, such as ¹¹C, ¹³C and ¹⁴C, chlorine, such as ³⁶Cl, fluorine, such as ¹⁸F, iodine, such as ¹²³I and ¹²⁵I, nitrogen, such as ¹³N and ¹⁵N, oxygen, such as ¹⁵O, ¹⁷O and ¹⁸O, phosphorus, such as ³²P, and sulfur, such as ³⁵S. Certain isotopically-labelled compounds of the compounds disclosed herein, such as those incorporating a radioactive isotope, are useful in drug and/or substrate tissue distribution studies. The radioactive isotopes tritium (³H) and carbon-14 (¹⁴C) are particularly useful for this purpose in view of their ease of incorporation and ready means of detection. Substitution with isotopes such as deuterium (²H or D) may afford certain therapeutic advantages resulting from greater metabolic stability, for example, increased in vivo half-life or reduced dosage requirements, and hence may be advantageous in some circumstances. As such, the term “deuterated” refers to the substitution of one or more hydrogen atoms with one or more deuterium atoms on a particular structure or functional group. Substitution with positron emitting isotopes, such as ¹¹C, ¹⁸F, ¹⁵O and ¹³N, can be useful in Positron Emission Topography (PET) studies, for example, for examining target occupancy. Isotopically-labelled compounds of the compounds disclosed herein can generally be prepared by conventional techniques known to those skilled in the art or by processes analogous to those described in the accompanying SYNTHETIC PROCEDURES and EXAMPLES described herein using an appropriate isotopically-labelled reagent in place of the non-labelled reagent previously employed.

The term “disease” or “disorder” as used herein refers to any condition that impairs the normal functioning of the body, such as a functional abnormality or disturbance that impairs normal functioning.

The term “combination therapy” means the administration of two or more therapeutic agents to treat a therapeutic condition or disorder described in the present disclosure.

“IGF-1R inhibitor” is used herein to refer to a compound that exhibits an IC₅₀ with respect to IGF-1R activity of no more than about 100 μM and more typically not more than about 50 μM, as measured in the IGF-1R assay described generally herein. “IC₅₀” is that concentration of inhibitor which reduces the activity of an enzyme (e.g., IGF-1R) to half-maximal level. Certain compounds disclosed herein have been discovered to exhibit inhibition against IGF-1R. In certain embodiments, compounds will exhibit an IC₅₀ with respect to IGF-1R of no more than about 2 μM; in yet further embodiments, compounds will exhibit an IC₅₀ with respect to IGF-1R of not more than about 1 μM; in yet further embodiments, compounds will exhibit an IC₅₀ with respect to IGF-1R of not more than about 500 nM, as measured in the IGF-1R assay described herein.

The phrase “therapeutically effective” is intended to qualify the amount of active ingredients used in the treatment of a disease or disorder or on the effecting of a clinical endpoint.

The term “therapeutically acceptable” refers to those compounds (or salts, prodrugs, tautomers, zwitterionic forms, etc.) which are suitable for use in contact with the tissues of patients without undue toxicity, irritation, and allergic response, are commensurate with a reasonable benefit/risk ratio, and are effective for their intended use.

As used herein, “treating,” “treatment,” and the like means ameliorating a disease, so as to reduce, ameliorate, or eliminate its cause, its progression, its severity, or one or more of its symptoms, or otherwise beneficially alter the disease in a subject. In certain embodiments, reference to “treating” or “treatment” of a subject at risk for developing a disease, or at risk of disease progression to a worse state, is intended to include prophylaxis. Prevention of a disease may involve complete protection from disease, or may involve prevention of disease progression. Prevention of diseases may also mean prevention of progression of a disease to a later stage of the disease.

The term “patient” is generally synonymous with the term “subject” and includes all mammals including humans. Examples of patients include humans, non-human primates such as chimpanzees, and other apes and monkey species; livestock such as cows, goats, sheep, pigs, and rabbits, and companion animals such as dogs, cats, rabbits, and horses. Preferably, the patient is a human.

### Pharmaceutical Compositions and Formulations

Formulations may be prepared by any suitable method, typically by uniformly mixing the active compound(s) with liquids or finely divided solid carriers, or both, in the required proportions and then, if necessary, forming the resulting mixture into a desired shape.

Conventional excipients, such as binding agents, fillers, acceptable wetting agents, tableting lubricants and disintegrants may be used in tablets and capsules for oral administration. Liquid preparations for oral administration may be in the form of solutions, emulsions, aqueous or oily suspensions and syrups. Alternatively, the oral preparations may be in the form of dry powder that can be reconstituted with water or another suitable liquid vehicle before use. Additional additives such as suspending or emulsifying agents, non-aqueous vehicles (including edible oils), preservatives and flavorings and colorants may be added to the liquid preparations. Parenteral dosage forms may be prepared by dissolving the compound provided herein in a suitable liquid vehicle and filter sterilizing the solution before filling and sealing an appropriate vial or ampule. These are just a few examples of the many appropriate methods well known in the art for preparing dosage forms.

A compound of the present invention can be formulated into pharmaceutical compositions using techniques well known to those in the art. Suitable pharmaceutically-acceptable carriers, outside those mentioned herein, are known in the art; for example, see Remington, The Science and Practice of Pharmacy, 20th Edition, 2000, Lippincott Williams & Wilkins. (Editors: Gennaro et. al.).

The compounds provided herein, together with a conventional adjuvant, carrier, or diluent, may thus be placed into the form of pharmaceutical formulations and unit dosages thereof and in such form may be employed as solids, such as tablets or filled capsules, or liquids such as solutions, suspensions, emulsions, elixirs, gels or capsules filled with the same, all for oral use, or in the form of sterile injectable solutions for parenteral (including subcutaneous) use. Such pharmaceutical compositions and unit dosage forms thereof may comprise conventional ingredients in conventional proportions, with or without additional active compounds or principles and such unit dosage forms may contain any suitable effective amount of the active ingredient commensurate with the intended daily dosage range to be employed.

For oral administration, the pharmaceutical composition may be in the form of, for example, a tablet, capsule, suspension or liquid. The pharmaceutical composition is preferably made in the form of a dosage unit containing a particular amount of the active ingredient. Examples of such dosage units are capsules, tablets, powders, granules or a suspension, with conventional additives such as lactose, mannitol, corn starch or potato starch; with binders such as crystalline cellulose, cellulose derivatives, acacia, corn starch or gelatins, with disintegrators such as corn starch, potato starch or sodium carboxymethyl-cellulose; and with lubricants such as talc or magnesium stearate. The active ingredient may also be administered by injection as a composition wherein, for example, saline, dextrose or water may be used as a suitable pharmaceutically acceptable carrier.

Compounds provided herein or a salt, solvate, or hydrate thereof can be used as active ingredients in pharmaceutical compositions, specifically as IGF-1R inhibitors. The term “active ingredient”, defined in the context of a “pharmaceutical composition”, refers to a component of a pharmaceutical composition that provides the primary pharmacological effect, as opposed to an “inactive ingredient” which would generally be recognized as providing no pharmaceutical benefit.

The dose when using the compounds provided herein can vary within wide limits and as is customary and is known to the physician or other clinician, it is to be tailored to the individual conditions in each individual case. It depends, for example, on the nature and severity of the illness to be treated, on the condition of the patient, on the compound employed or on whether an acute or chronic disease state is treated, or prophylaxis conducted, or on whether further active compounds are administered in addition to the compounds provided herein. Representative doses include, but are not limited to, about 0.001 mg to about 5000 mg, about 0.001 mg to about 2500 mg, about 0.001 mg to about 1000 mg, about 0.001 mg to about 500 mg, about 0.001 mg to about 250 mg, about 0.001 mg to 100 mg, about 0.001 mg to about 50 mg and about 0.001 mg to about 25 mg. Multiple doses may be administered during the day, especially when relatively large amounts are deemed to be needed, for example 2, 3, or 4 doses. Depending on the individual and as deemed appropriate from the healthcare provider it may be necessary to deviate upward or downward from the doses described herein.

The amount of active ingredient, or an active salt or derivative thereof, required for use in treatment will vary not only with the particular salt selected but also with the route of administration, the nature of the condition being treated and the age and condition of the patient and will ultimately be at the discretion of the attendant physician or clinician. In general, one skilled in the art understands how to extrapolate in vivo data obtained in a model system, typically an animal model, to another, such as a human. In some circumstances, these extrapolations may merely be based on the weight of the animal model in comparison to another, such as a mammal, preferably a human, however, more often, these extrapolations are not simply based on weights, but rather incorporate a variety of factors. Representative factors include the type, age, weight, sex, diet and medical condition of the patient, the severity of the disease, the route of administration, pharmacological considerations such as the activity, efficacy, pharmacokinetic and toxicology profiles of the particular compound employed, whether a drug delivery system is utilized, on whether an acute or chronic disease state is being treated, or prophylaxis conducted, or on whether further active compounds are administered in addition to the compounds provided herein and as part of a drug combination. The dosage regimen for treating a disease condition with the compounds and/or compositions provided herein is selected in accordance with a variety factors as cited above. Thus, the actual dosage regimen employed may vary widely and therefore may deviate from a preferred dosage regimen and one skilled in the art will recognize that dosage and dosage regimen outside these typical ranges can be tested and, where appropriate, may be used in the methods provided herein.

Liquid form preparations include solutions, suspensions, and emulsions, for example, water or water-propylene glycol solutions. For example, parenteral injection liquid preparations can be formulated as solutions in aqueous polyethylene glycol solution. Injectable preparations, for example, sterile injectable aqueous or oleaginous suspensions may be formulated according to the known art using suitable dispersing or wetting agents and suspending agents.

Aqueous formulations suitable for oral use can be prepared by dissolving or suspending the active component in water and adding suitable colorants, flavors, stabilizing and thickening agents, as desired.

Also included are solid form preparations which are intended to be converted, shortly before use, to liquid form preparations for oral administration. Such liquid forms include solutions, suspensions, and emulsions. These preparations may contain, in addition to the active component, colorants, flavors, stabilizers, buffers, artificial and natural sweeteners, dispersants, thickeners, solubilizing agents and the like.

For topical administration to the epidermis the compounds provided herein may be formulated as ointments, creams, or lotions, or as a transdermal patch.

Ointments and creams may, for example, be formulated with an aqueous or oily base with the addition of suitable thickening and/or gelling agents. Lotions may be formulated with an aqueous or oily base and will in general also contain one or more emulsifying agents, stabilizing agents, dispersing agents, suspending agents, thickening agents, or coloring agents.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules and powders in vials or ampoules. Also, the unit dosage form can be a capsule, tablet, cachet, or lozenge itself, or it can be the appropriate number of any of these in packaged form.

Tablets or capsules for oral administration and liquids for intravenous administration are preferred compositions.

### Indications and Methods of Treatment

Also provided herein are methods for treating IGF-1R-mediated disorders in a mammal in need of such treatment comprising administering to the mammal an amount of a compound disclosed herein effective to halt the further progression of, reduce the severity of, undo the effect of, or prevent the return of the disease or disorder in the subject. In a related aspect, certain embodiments provide therapeutic compositions comprising at least one compound disclosed herein in combination with one or more additional agents for the treatment of IGF-1R-mediated disorders.

Specific diseases to be treated by the compounds, compositions, and methods disclosed herein include thyroid eye disease (TED), also known as thyroid-associated ophthalmopathy (TAO), or Graves' ophthalmopathy or orbitopathy (GO).

Besides being useful for human treatment, certain compounds and formulations disclosed herein may also be useful for veterinary treatment of companion animals (e.g., horses, dogs, and cats), exotic animals farm animals, rodents, and the like.

Accordingly, the present invention also relates to a method of inhibiting at least one IGF-1R function comprising the step of contacting IGF-1R with a compound as described herein. The cell phenotype, cell proliferation, activity of IGF-1R, change in biochemical output produced by active IGF-1R, expression of IGF-1R, or binding of IGF-1R with a natural binding partner may be monitored. Such methods may be modes of treatment of disease, biological assays, cellular assays, biochemical assays, or the like.

Also provided herein is a method of treatment of a IGF-1R-mediated disease comprising the administration of a therapeutically effective amount of a compound as disclosed herein, or a salt thereof, to a patient in need thereof.

### Thyroid Eye Disease (TED)

As used herein, “Thyroid Eye Disease” (TED), “Thyroid-associated Ophthalmopathy” (TAO), “Thyroid Inflammatory Eye Disease (TIED),” “Graves' Ophthalmopathy” (GO) or “Graves' Orbitopathy” (GO) refer to the same disease or disorder. These terms refer to the inflammatory orbital pathology associated with some autoimmune thyroid disorders, most commonly with “Graves' Disease” (GD), but sometimes with other diseases, e.g., Hashimoto's thyroiditis.

The cause of TED is unknown. TED is typically associated with Graves' hyperthyroidism but can also occur as part of other autoimmune conditions that affect the thyroid gland and produce pathology in orbital and periorbital tissue, and, rarely, the pretibial skin (pretibial myxedema) or digits (thyroid acropachy). TED is an autoimmune orbitopathy in which the orbital and periocular soft tissues are primarily affected with secondary effects on the eye and vision. In TED, as a result of inflammation and expansion of orbital soft tissues, primarily eye muscles and adipose, the eyes are forced forward (bulge) out of their sockets—a phenomenon termed proptosis or exophthalmos.

The annual incidence rate of TED has been estimated at 16 cases per 100,000 women and 2.9 cases per 100.000 men from a study based in one largely rural Minnesota community. There appears to be a female preponderance in which women are affected 2.5-6 times more frequently than men; however, severe cases occur more often in men than in women. In addition, most patients are aged 30-50 years, with severe cases appearing to be more frequent in those older than 50 years. Although most cases of TED do not result in loss of vision, this condition can cause vision-threatening exposure keratopathy, troublesome diplopia (double vision), and compressive dysthyroid optic neuropathy.

TED may precede, coincide with, or follow the systemic complications of dysthyroidism. The ocular manifestations of TED include upper eyelid retraction, lid lag, swelling, redness (erythema), conjunctivitis, and bulging eyes (exophthalmos or proptosis), chemosis, periorbital edema, and altered ocular motility with significant functional, social, and cosmetic consequences.

Many of the signs and symptoms of TED, including proptosis and ocular congestion, result from expansion of the orbital adipose tissue and periocular muscles. The adipose tissue volume increases owing in part to new fat cell development (adipogenesis) within the orbital fat. The accumulation of hydrophilic glycosaminoglycans, primarily hyaluronic acid, within the orbital adipose tissue and the perimysial connective tissue between the extraocular muscle fibers, further expands the fat compartments and enlarges the extraocular muscle bodies. Hyaluronic acid is produced by fibroblasts residing within the orbital fat and extraocular muscles, and its synthesis in vitro is stimulated by several cytokines and growth factors, including IL-1β, interferon-γ, platelet-derived growth factor, thyroid stimulating hormone (TSH), and insulin-like growth factor 1 (IGF-1).

TED is commonly considered to be the autoimmune orbital manifestation of Graves' Disease (GD). However, only approximately 30% of patients with Graves' hyperthyroidism manifest clinically relevant ocular pathology indicating there is mechanistic heterogeneity and differentiation between the conditions. The molecular mechanisms underlying TED remain unclear. It is accepted that the generation of autoantibodies that act as agonists on the thyroid-stimulating hormone receptor (TSHR) is responsible for Graves' hyperthyroidism. Pathogenic overstimulation of TSHR, leads to overproduction of thyroid hormones (T3 and T4) and accelerated metabolism of many tissues.

Antibodies that activate the insulin-like growth factor 1 receptor (IGF-1R) have also been detected and implicated in active TED. Without being bound to any theory, it is believed that TSHR and IGF-1R form a physical and functional complex in orbital fibroblasts, and that blocking IGF-1R appears to attenuate both IGF-1 and TSH-dependent signaling. It has been suggested that blocking IGF-1R using an antibody antagonist might reduce both TSHR- and IGF-1-dependent signaling and therefore interrupt the pathological activities of autoantibodies acting as agonists on either receptor.

Immunoglobulins that activate IGF-1R signaling have been detected in patients with GD and TED. Furthermore, IGF-1 synergistically enhances the actions of thyrotropin. IGF-1R, a membrane-spanning tyrosine kinase receptor with roles in development and metabolism, also stimulates immune function and thus might be targeted therapeutically in autoimmune diseases. IGF-1R is overexpressed by orbital fibroblasts and by T cells and B cells in persons with GD and TED. It forms a signaling complex with TSHR through which it is transactivated. In vitro studies of orbital fibroblasts and fibrocytes show that IGF-1R-inhibitory antibodies can attenuate the actions of IGF-1, thyrotropin, thyroid-stimulating immunoglobulins, and immunoglobulins isolated from patients with GD and TED. These observations prompted a trial of teprotumumab, a fully human IGF-1R-inhibitory monoclonal antibody, in patients with active, moderate-to-severe TED.

The terms “proptosis” and “exophthalmos” (also known as exophthalmus, exophthalmia, or exorbitism) refer to the forward projection, displacement, bulging, or protrusion of an organ. As used herein, the terms refer to the forward projection, displacement, bulging, or protrusion of the eye anteriorly out of the orbit. Proptosis and exophthalmos are considered by some of skill in the art to have the same meaning and are often used interchangeably, while others attribute subtle differences to their meanings. Exophthalmos is used by some to refer to severe proptosis; or to refer to endocrine-related proptosis. Yet others use the term exophthalmos when describing proptosis associated with the eye, in, for example, subjects with TED (TAO or GO).

As used herein, the terms “proptosis” and “exophthalmos” are used interchangeably and refer to the forward projection, displacement, bulging, or protrusion of the eye anteriorly out of the orbit. Owing to the rigid bony structure of the orbit with only anterior opening for expansion, any increase in orbital soft tissue contents taking place from the side or from behind will displace the eyeball forward. Proptosis or exophthalmos can be the result of a several disease processes including infections, inflammations, tumors, trauma, metastases, endocrine lesions, vascular diseases & extra orbital lesions. TED (TAO or GO) is currently recognized as the most common cause of proptosis in adults. Exophthalmos can be either bilateral, as is often seen in TED (TAO or GO), or unilateral (as is often seen in an orbital tumor).

Measurement of the degree of exophthalmos can be performed using an exophthalmometer, an instrument used for measuring the degree of forward displacement of the eye. The device allows measurement of the forward distance of the lateral orbital rim to the front of the cornea.

Computed tomography (CT) scanning and Magnetic resonance imaging (MRI) may also be used in evaluating the degree of exophthalmos or proptosis. CT scanning is an excellent imaging modality for the diagnosis of TED (TAO or GO). In addition to allowing visualization of the enlarged extraocular muscles, CT scans provide the surgeon or clinician with depictions of the bony anatomy of the orbit when an orbital decompression is required. MRI, with its multi-planar and inherent contrast capabilities, provides excellent imaging of the orbital contents without the radiation exposure associated with CT scan studies. MRI provides better imaging of the optic nerve, orbital fat, and extraocular muscle, but CT scans provide better views of the bony architecture of the orbit.

Orbital ultrasonography can also be a used for the diagnosis and evaluation of TED (TAO or GO), because it can be performed quickly and with a high degree of confidence. High reflectivity and enlargement of the extraocular muscles are assessed easily, and serial ultrasonographic examinations can also be used to assess progression or stability of the ophthalmopathy.

Based on the technologies currently available, or that will become available in the future, one of skill in the art would be capable of determining the best modality for diagnosing and evaluating the extent of proptosis or exophthalmos.

Although it is generally accepted that the normal range of proptosis is 12-21 mm, it must be noted that the value for a normal person varies by age, gender and race. For example, in normal adult white males, the average distance of globe protrusion is 16.5 mm, with the upper limit of normal at 21.7 mm. In adult African Americans it averages 18.2 mm, with an upper normal limit of 24.1 mm in males and 22.7 mm in females. In Mexican adults, males averaged 15.2 mm and females averaged 14.8 mm and in Iran, for the age group of 20-70 years, the average was 14.7 mm. In Taiwanese adults, comparing normal subjects to those with Graves' Ophthalmopathy, the normal group had an average reading of 13.9 mm versus 18.3 mm for the TED group.

Even within a group of people, there can be variability. Four ethnic groups in Southern Thailand had exophthalmometry measurement averages ranging from 15.4 mm to 16.6 mm. In 2477 Turkish patients, the median measurement was 13 mm, with an upper limit of 17 mm; and in a Dutch study, the upper limit was 20 mm in males and 16 mm in females.

Although the average and upper limits for exophthalmos or proptosis vary widely, it is accepted in the field that a difference greater than 2 mm between the eyes is significant and not normal.

One of skill in the art, for example an ophthalmologist, surgeon or other clinician skilled in the knowledge and treatment of eye disorders would know what a normal value of proptosis is based on the age, gender and race of the subject and have the ability to diagnose or evaluate the presence or absence of proptosis as well as track its progression.

### Activity Measures or Assessments

Several classification systems have been conceived to assess the clinical manifestations of TED (TAO or GO). In 1969, Werner reported the NOSPECS Classification (No physical signs or symptoms, Only signs, Soft tissue involvement, Proptosis, Extraocular muscle signs, Corneal involvement, and Sight loss) (Werner, S. C. American Journal of Ophthalmology, 1969, 68, no. 4, (46-648.)

The modified NOSPECS was also published by Werner in 1977 and has been broadly used since then (Weiner, S. C. American Journal of Ophthalmology, 1977, 83, no. 5, 725-727). This classification grades for clinical severity and does not provide a means of distinguishing active TED (inflammatory progressive) from inactive TED (noninflammatory stationary). Therefore, the indication for treatments used to be based exclusively in the severity of symptoms without consideration whether the disease was active or inactive. In 1989, Mourits et al. described the Clinical Activity Score (CAS) (Mourits et al., British Journal of Ophthalmology, 1989, 73, no. 8, 639-644) as a way of assessing the degree of active disease. This score, based on the classical signs of acute inflammation (pain, redness, swelling, and impaired function) was proposed as a clinical classification to discriminate easily between active and inactive disease and was modified in 1997 (Mounts et al., Clinical Endocrinology, 1997. 47, no. 1, 9-14). This protocol is further described below.

As used herein, the term CAS refers to the protocol described and scored as disclosed below. According to this protocol, one point is given for the presence of each of the parameters assessed in the list below. The sum of all points defines clinical activity and provides the CAS. For patients assessed for the first time only items 1-7 are scored. A CAS≥3/7 indicates active GO. For patients that are assessed for the second or subsequent time (typically, 1-3 months later), items 8-10 are also scored; and a CAS≥4/10 indicates active disease. A ten-item CAS scale exists as well, but in clinical trials, the 7-item scale is generally used, being more amenable to longitudinal studies involving multiple assessments.

The CAS consists of seven components:
1. spontaneous retrobulbar pain,
2. pain on attempted eye movements (upward, side-to-side, and downward gazes),
3. conjunctival redness,
4. redness of the eyelids,
5. chemosis (conjunctival swelling/edema),
6. swelling of the caruncle/plica, and
7. swelling of the eyelids.

Each component is scored as present (1 point) or absent (0 points). The score at each efficacy assessment is the sum of all items present; giving a range of 0-7, where 0 or 1 constitutes inactive disease and 7 severe active ophthalmopathy. A change of >2 points is considered clinically meaningful.

Item 1, spontaneous orbital pain could be a painful, or oppressive feeling on, or behind, the globe. This pain may be caused by the rise in intraorbital pressure, when the orbital tissues volume increases through excess synthesis of extracellular matrix, fluid accumulation, and cellular infiltration and expansion. Item 2, gaze evoked orbital pain, could be pain in the eyes when looking, or attempting to look, up, down or sideways. i.e., pain with upward, downward, or lateral eye movement, or when attempting upward, downward, or lateral gaze. This kind of pain could arise from the stretching of the inflamed muscle(s), especially on attempted up-gaze. The ‘stretching pain’ cannot be provoked by digital pressing on the eyeball, as would be expected if it were a manifestation of the raised intraorbital pressure. Both kinds of pain can be reduced after anti-inflammatory treatment. These kinds of pain are therefore considered to be directly related to autoimmune inflammation in the orbit and thus useful in assessing TED activity.

Swelling in TED (TAO or GO) is seen as chemosis (edema of the conjunctiva) and swelling of the caruncule and/or plica semilunaris. Both are signs of TED activity. Swollen eyelids can be caused by edema, fat prolapse through the orbital septum, or fibrotic degeneration. In addition to swelling, other symptoms indicative of active TED include redness and/or pain of the conjunctiva, eyelid, caruncule and/or plica semilunaris.

Other grading systems have also been developed for the assessment of TED (TAO or GO). The VISA Classification (vision, inflammation, strabismus, and appearance) (Dolman, P. J., and Rootman, J., Ophthalmic Plastic and Reconstructive Surgery, 2006, 22, no. 5, 319-324 and Dolman, P. J., Best Practice & Research Clinical Endocrinology & Metabolism, 2012, 26, no. 3, 229-248) and the European Group of Graves' Orbitopathy (EUGOGO) Classification (Bartalena, L., et al., European Journal of Endocrinology, 2008, 158, no. 3, 273-285) are two such examples. Both systems are grounded in the NO SPECS and CAS classifications and use indicators to assess the signs of activity and the degree of severity. More importantly, they allow the clinician to guide the treatment of the patient with GO. VISA is more commonly used in North America and Canada while EUGOGO is in Europe. Since the VISA and EUGOGO protocols are not interchangeable, only one of them should be employed as a reference in a specific patient.

### Graves Ophthalmopathy Quality of Life (GO-QoL)

In addition to proptosis (or exophthalmos) and CAS, quality of life (QoL) was also evaluated with the use of the Graves' ophthalmopathy quality of life (GO-QoL) questionnaire. This questionnaire is designed to determine the improved quality of life after treatment. In some embodiments, questionnaire may determine the decreased or lack of side effects after being treated with compound disclosed herein, or a pharmaceutically acceptable salt or solvate thereof, according to the methods disclosed herein, as compared to treatment with glucocorticoids.

The GO-QoL questionnaire has two self-assessment subscales. The first relates to the impact of visual function on daily activities, while the second relates to the impact of self-perceived appearance. Each subscale has 8 questions which are answered with: (i) yes—very much so; (ii) yes—a little; or (iii) no—not at all. Each question is scored 0-2, respectively, and the total raw score is then mathematically transformed to a 0-100 scale, where 0 represents the most negative impact on quality of life, and 100 represents no impact. A change of >8 points on the 0-100 scale is considered to be clinically meaningful. The combined score takes raw scores from both subscales and again transforms them to a single 0-100 scale.

### Severity Measures

For lid aperture, the distance between the lid margins are measured (in mm) with the patient looking in the primary position, sitting relaxed, and with distant fixation. For swelling of the eyelids, the measure/evaluation is either “absent/equivocal,” “moderate,” or “severe.” Redness of the eyelids is either absent or present. Redness of the conjunctivae is either absent or present. Conjunctival edema is either absent or present. Inflammation of the caruncle or plica is either absent or present. Exophthalmos was measured in millimeter using the same Hertel exophthalmometer and same intercanthal distance for an individual patient. Subjective diplopia is scored from 0 to 3 (0=no diplopia; 1=intermittent, i.e., diplopia in primary position of gaze, when tired or when first awakening; 2=inconstant, i.e., diplopia at extremes of gaze; 3=constant, i.e., continuous diplopia in primary or reading position). For eye muscle involvement, the ductions are measured in degrees. Corneal involvement is either absent/punctate or keratopathy/ulcer. For optic nerve involvement, i.e., best-corrected visual acuity, color vision, optic disc, relative afferent pupillary defect, the condition is either absent or present. In addition, visual fields are checked if optic nerve compression was suspected.

### Severity Classification

Sight-threatening thyroid eye disease; Patients with dysthyroid optic neuropathy (DON) and/or corneal breakdown. This category warranted immediate intervention.

Moderate-to-severe thyroid eye disease: Patients without sight-threatening disease whose eye disease had sufficient impact on daily life to justify the risks of immunosuppression (if active) or surgical intervention (if inactive). Patients with moderate-to-severe thyroid eye disease usually had any one or more of the following: lid retraction≥2 mm, moderate or severe soft tissue involvement, exophthalmos≥3 mm above normal for race and gender, inconstant or constant diplopia.

Mild thyroid eye disease: Patients whose features of thyroid eye disease have only a minor impact on daily life insufficient to justify immunosuppressive or surgical treatment. They usually have only one or more of the following; minor lid retraction (<2 mm), mild soft tissue involvement, exophthalmos<3 mm above normal for race and gender, transient or no diplopia, and corneal exposure responsive to lubricants.

### Assessment of Gorman Grading of Diplopia

The Gorman assessment of subjective diplopia includes four categories: no diplopia (absent), diplopia when the patient is tired or awakening (intermittent), diplopia at extremes of gaze (inconstant), and continuous diplopia in the primary or reading position (constant). Patients are scored according to which grade of diplopia they are experiencing. An improvement of ≥1 grade is considered clinically meaningful.

Additional testing, including clinical trial protocols and criteria and the lead-in study, which can be performed to determine efficacy for the treatment of TED can be found in US20190225696A1.

Further, the IGR-1R inhibitors described herein may be useful for the treatment of TED in subjects who were either proptosis non-responders (<2 mm reduction in proptosis in the study eye) in the lead-in study or were proptosis responders in the lead-in study but meet the criteria for re-treatment due to relapse.

### Methods of Treatment

Accordingly, provided herein are methods for treating IGF-1R-mediated disorders in a mammal in need of such treatment comprising administering to said subject an amount of a compound disclosed herein effective to reduce or prevent said disorder in the subject. In a related aspect, certain embodiments provide therapeutic compositions comprising at least one compound disclosed herein in combination with one or more additional agents for the treatment of IGF-1R-mediated disorders. Specific diseases to be treated by the compounds, compositions, and methods disclosed herein include thyroid eye disease (TED), also known as thyroid-associated ophthalmopathy (TAO), or Graves' ophthalmopathy or orbitopathy (GO).

In some embodiments, the disease is thyroid eye disease (TED).

Also provided herein is a compound disclosed herein, or a pharmaceutically acceptable salt or solvate thereof, for use as a medicament.

Also provided herein is a compound disclosed herein, or a pharmaceutically acceptable salt or solvate thereof, for use as a medicament for the treatment of a IGF-1R-mediated disease.

Also provided is a compound disclosed herein, or a pharmaceutically acceptable salt or solvate thereof, for use in the manufacture of a medicament for the treatment of a IGF-1R-mediated disease.

Also provided is the use of a compound disclosed herein, or a pharmaceutically acceptable salt or solvate thereof, as disclosed herein for the treatment of a IGF-1R-mediated disease.

Also provided herein is a method of inhibition of IGF-1R comprising contacting IGF-1R with a compound as disclosed herein, or a pharmaceutically acceptable salt or solvate thereof.

### Combination Therapy

In certain instances, it may be appropriate to administer at least one of the compounds described herein (or a pharmaceutically acceptable salt, ester, or prodrug thereof) in combination with another therapeutic agent.

### EXAMPLES

The following examples are provided for illustrative purposes only and not to limit the scope of the claims provided herein. All IUPAC names were generated using CambridgeSoft's ChemDraw Professional 22.0.0.22.

### R. I. Chemical Synthesis

As used above, and throughout the description of the invention, the following abbreviations, unless otherwise indicated, shall be understood to have the following meanings:

### List of Commonly Used Abbreviations

Ac (acetyl);
ACN, CH₃CN (acetonitnile);
AcOH, HOAc (acetic acid);
atm (atmosphere);
BH₃-DMS complex, BMS (borane dimethylsulfide);
BINAP (2,2′-bis(diphenylphosphino)-1,1′-binaphthyl);
Boc (tert-butoxycarbonyl);
Boc₂O (di-tert-butyl dicarbonate);
BuLi, n-BuLi (n-buyllithium);
DCM (dichloromethane);
DIPEA (diisopropylethylamine);
DMAP (4-dimethylaminopyridine);
DME (dimethyl ether).
DMF (N,N-dimethylformamide);
DMSO (dimethylsulfoxide);
equiv. (equivalents);
Et (ethyl);
Et₃N, TEA (triethylamine);
EtOAc (ethyl acetate);
EtOH (ethanol);
g (grams); mg (milligrams); μg (micrograms);
h, hr, hrs (hours);
HCl (hydrochloric acid);
HFIP (hexafluoro-2-propanol, hexafluoroisopropanol);
HPLC (high-performance liquid chromatography);
IGF-1R (insulin like growth factor 1 receptor);
i-Pr (isopropyl);
i-PrOH, IPA (isopropanol);
L (liters); mL (milliliters); μL (microliters);
LAH (lithium aluminum hydride);
LC-MS (liquid chromatography-mass spectrometry);
LDA (lithium diisopropylamide);
M (molar); mM (millimolar); μM (micromolar);
Me (methyl);
Mel (methyl iodide, iodomethane);
MeLi (methyllithium);
MeOH (methanol);
min, mins (minute(s));
mol (moles); mmol (millimoles); μmol (micromoles);
MS (mass spectrometry);
MsCl (methanesulfonyl chloride, mesyl chloride);
N₂(g) (nitrogen gas);
NaOtBu (sodium tert-butoxide);
NBS (N-bromosuccinimide);
NMP (N-methyl-2-pyrrolidine)
NMR (nuclear magnetic resonance);
Pd(OAc)₂ (palladium(II) acetate);
Pd₂(dba)₃ (tris(dibenzylideneacetone)dipalladium(0));
PE (petroleum ether);
PhMe (toluene);
Pr (propyl);
psi (pounds per square inch);
PTFE (polytetrafluoroethylene, Teflon-1);
PyBrop (bromo-tris-pyrrolidino-phosphonium hexafluorophosphate);
RBF (round bottom flask);
Rt, RT (room temperature);
SiO₂ (silica);
SOCl₂ (thionyl chloride)
TBABr (tetrabutylammonium bromide);
t-Bu (tert-butyl);
tBu-Josiphos ((R)-(−)-1-[(S)-2-(dicyclohexyl-phosphino)ferrocenyl]ethyl-di-t-butyl phosphine);
tBuOH (tert-butyl alcohol, tert-butanol);
TCFH (N′-tetramethylformamidinium hexafluorophosphate);
THF (tetrahydrofuran);
TLC (thin layer chromatography);
ρW (microwave);
vol. (volume equivalents);
Xantphos (4,5-Bis(diphenylphosphino)-9,9-dimethylxanthene).

### Synthetic Methods

The following intermediates and examples can be synthesized using the general synthetic procedures set forth in the following Examples.

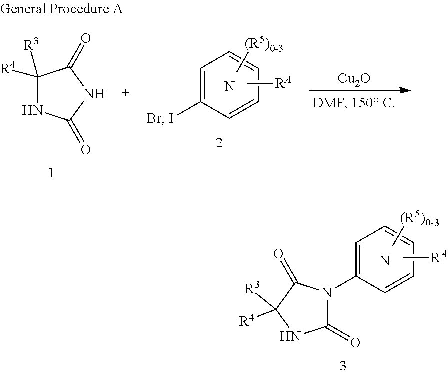

A mixture of hydantoin 1 (1.0 equiv.), aryl halide 2 (1.0 equiv.) and Cu₂O (1.0 equiv.) in DMF (0.4 M) was heated at 150° C. under N₂ for 16 h. After cooling to rt, DMF was removed under reduced pressure. Water (15 vol.) was added and the resulting slurry was stirred at rt for 30 min. Then, NH₄OH (5 vol.) was added and stirring continued for 30 min. The precipitate was collected by filtration, washed with water, and dried under vacuum. The hydantoin product 3 was generally >95% pure, or further purified by flash chromatography (SiO₂, heptanes/EtOAc) as needed.

The following compounds are prepared according to General Procedure A.

| Name | Structure | MS (m/z) |
|---|---|---|
| 5,5-dimethyl-3-(4- (trimethylsilyl)phenyl) imidazolidine-2,4-dione | 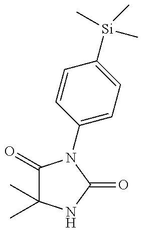 [SMILES: CC1(C)NC(=O)N(c2ccc([Si](C)(C)C)cc2)C1=O] | [M + H]⁺ = 277.0 |
| 5,5-dimethyl-3-(4- (3-methyloxetan-3- yl)phenyl)imidazolidine- 2,4-dione | 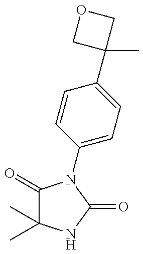 [SMILES: CC1(C)NC(=O)N(c2ccc(C3(C)COC3)cc2)C1=O] | [M − H]⁻ = 273.0 |
| 3-(4-(tert-butyl)phenyl)- 5,5-dimethylimidazolidine- 2,4-dione | 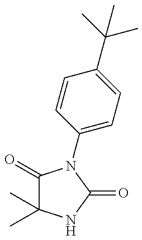 [SMILES: CC1(C)NC(=O)N(c2ccc(C(C)(C)C)cc2)C1=O] | [M − H]⁻ = 261.0 |
| 5,5-dimethyl-3-(4-(1- (trifluoromethyl)cyclopropyl) phenyl)imidazolidine- 2,4-dione | 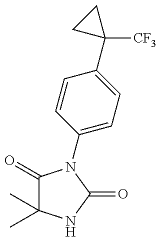 [SMILES: CC1(C)NC(=O)N(c2ccc(C3(C(F)(F)F)CC3)cc2)C1=O] | [M − H]⁻ = 311.0 |
| (1-(4-(4,4-dimethyl- 2,5-dioxoimidazolidin-1- yl)phenyl)cyclopropane- 1-carbonitrile) | 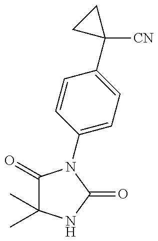 [SMILES: CC1(C)NC(=O)N(c2ccc(C3(C#N)CC3)cc2)C1=O] | [M − H]⁻ = 268.2 |
| 3-(5-(tert-butyl) pyridin-2-yl)-5,5- dimethylimidazolidine- 2,4-dione | 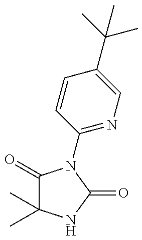 [SMILES: CC1(C)NC(=O)N(c2ccc(C(C)(C)C)cn2)C1=O] | [M + H]⁺ = 262.2 |

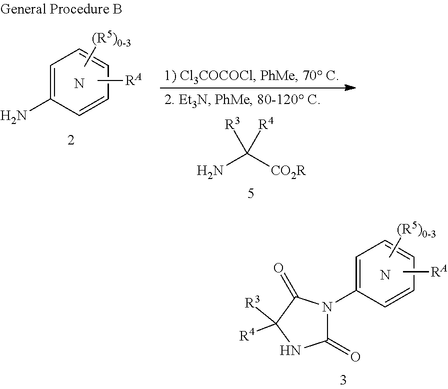

Step 1: A mixture of an appropriately substituted aryl or heteroaryl aniline 4 (1 equiv.) and toluene (0.05-0.1 M) was cooled in an ice bath with stirring. Diphosgene (1 equiv.) was added dropwise. The mixture was allowed to warm to room temperature and then was heated to 70° C. until LC-MS analysis showed full conversion to the corresponding isocyanate (˜3 h).

Step 2: The mixture from Step 1 was cooled to room temperature and triethylamine (3 equiv.) and a substituted amino-ester 5 (1.5 equiv.) were added with stirring. The reaction was heated (80-120° C.) for 6 h with stirring in a sealed tube. The reaction was cooled to ambient temperature and diluted with EtOAc. The organic layer was washed with saturated aqueous NH₄Cl, saturated aqueous NaHCO, then brine. The organic layer was separated, dried over sodium sulfate, filtered, and concentrated in vacuo. This crude material was generally used as-is in the next step, or purified by flash chromatography (SiO₂, Heptanes/EtOAc) as needed affording the corresponding hydantoin product 3.

The following compounds are prepared according to General Procedure B.

| Name | Structure | MS (m/z) |
|---|---|---|
| 1-(4-(4,4-dimethyl-2,5-dioxoimidazolidin-1- yl)phenyl)cyclopropanecarbonitrile | 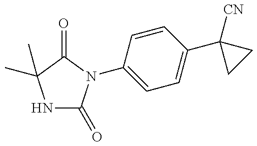 [SMILES: CC1(C)NC(=O)N(c2ccc(C3(C#N)CC3)cc2)C1=O] | [M − H]⁻ = 311 |
| 3-(6-(tert-butyl)pyridin-3-yl)-5,5- dimethylimidazolidine-2,4-dione | 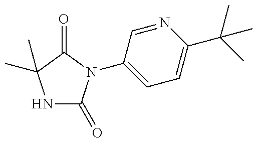 [SMILES: CC1(C)NC(=O)N(c2ccc(C(C)(C)C)nc2)C1=O] | [M + H]⁻ = 262.2 |
| 5,5-dimethyl-3-(4-(1- (trifluoromethyl)cyclopropyl)phenyl) imidazolidine-2,4-dione | 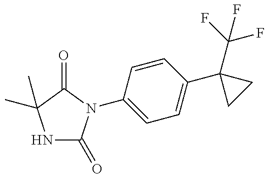 [SMILES: CC1(C)NC(=O)N(c2ccc(C3(C(F)(F)F)CC3)cc2)C1=O] | [M + H]⁻ = 311.1 |
| 5,5-dimethyl-3-(4-(pentafluoro-l6- sulfaneyl)phenyl)imidazolidine-2,4-dione | 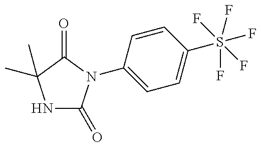 [SMILES: CC1(C)NC(=O)N(c2ccc(S(F)(F)(F)(F)F)cc2)C1=O] | [M − H]⁻ = 329.4 |
| 1-(4-(4,4-dimethyl-2,5-dioxoimidazolidin-1- yl)phenyl)cyclobutane-1-carbonitrile | 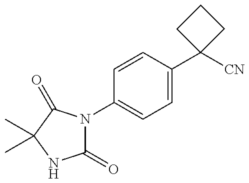 [SMILES: CC1(C)NC(=O)N(c2ccc(C3(C#N)CCC3)cc2)C1=O] | [M − H]⁻ 282.2 |
| 3-(3-(tert-butyl)-1-methyl-1H-pyrazol-5-yl)-5,5- dimethylimidazolidine-2,4-dione | 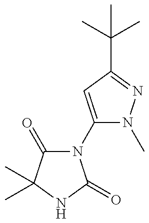 | [M + H]⁺ = 265 |

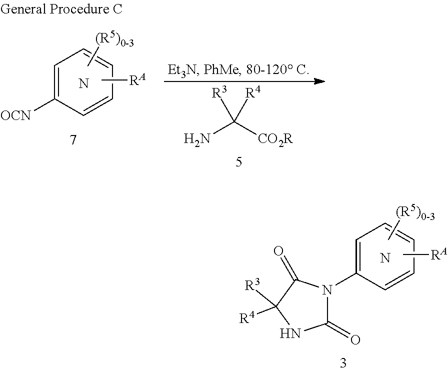

Commercially available isocyanate 7 (1 equiv.) was stirred in toluene (0.05-0.1 M) at room temperature and triethylamine (3 equiv.) and a substituted amino-ester 5 (1.5 equiv.) were added with stirring. The reaction was heated to (80-120° C.) for 6 h with stirring in a sealed tube. The reaction was cooled to ambient temperature and diluted with EtOAc. The organic layer was washed with saturated aqueous NH₄Cl, saturated aqueous NaHCO₃, and brine. The organic layer was separated, dried over sodium sulfate, filtered, and concentrated in vacuo. This crude material was generally used as-is in the next step, or purified by flash chromatography (SiO₂, Heptanes/EtOAc) as needed affording the corresponding hydantoin product 3.

The following compounds are prepared according to General Procedure C.

| Name | Structure | MS (m/z) |
|---|---|---|
| 5,5-dimethyl-3-(4- ((trifluoromethyl)thio)phenyl)imidazolidine-2,4- dione | 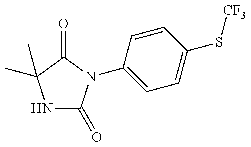 [SMILES: CC1(C)NC(=O)N(c2ccc(SC(F)(F)F)cc2)C1=O] | [M − H]⁻ = 303.4 |
| 5,5-dimethyl-3-(4- ((trifluoromethyl)sulfinyl)phenyl)imidazolidine- 2,4-dione *obtained from oxone oxidation of above compound | 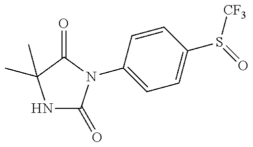 [SMILES: CC1(C)NC(=O)N(c2ccc(S(=O)C(F)(F)F)cc2)C1=O] | [M + H]⁺ = 319 |
| 5,5-dimethyl-3-(4- ((trifluoromethyl)sulfonyl)phenyl)imidazolidine- 2,4-dione *obtained from oxone oxidation of above compound | 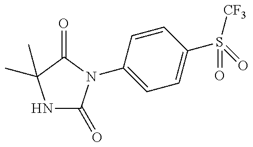 [SMILES: CC1(C)NC(=O)N(c2ccc(S(=O)(=O)C(F)(F)F)cc2)C1=O] | [M + H]⁺ = 335 |
| 5,5-dimethyl-3-(4- (trifluoromethoxy)phenyl)imidazolidine-2,4- dione | 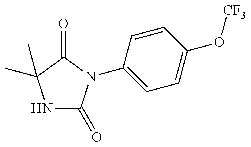 [SMILES: CC1(C)NC(=O)N(c2ccc(OC(F)(F)F)cc2)C1=O] | [M − H]⁻ = 287.0 |
| 3-(4-bromophenyl)-5,5-dimethylimidazolidine- 2,4-dione | 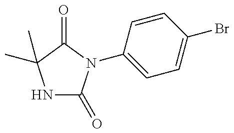 [SMILES: CC1(C)NC(=O)N(c2ccc(Br)cc2)C1=O] | [M + H]⁺ = 283.0 |
| 3-(4-acetylphenyl)-5,5-dimethylimidazolidine- 2,4-dione | 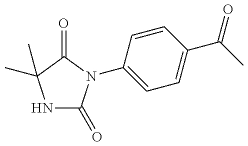 [SMILES: CC(=O)c1ccc(N2C(=O)NC(C)(C)C2=O)cc1] | [M + H]⁺ = 247.1 |
| 6-(4-((trifluoromethyl)thio)phenyl)-4,6- diazaspiro[2.4]heptane-5,7-dione | 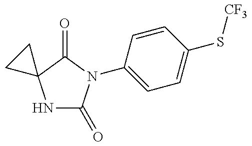 [SMILES: O=C1NC2(CC2)C(=O)N1c1ccc(SC(F)(F)F)cc1] | [M + H]⁺ = 301 |
| 3-(4-(tert-butyl)phenyl)-5,5- dimethylimidazolidine-2,4-dione | 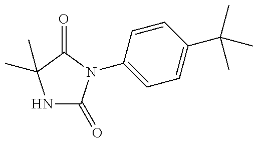 [SMILES: CC1(C)NC(=O)N(c2ccc(C(C)(C)C)cc2)C1=O] | [M + H]⁺ = 261.0 |
| 5-ethyl-5-methyl-3-(4- ((trifluoromethyl)thio)phenyl)imidazolidine-2,4- dione | 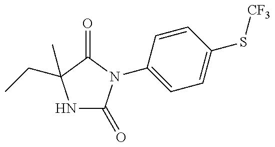 [SMILES: CCC1(C)NC(=O)N(c2ccc(SC(F)(F)F)cc2)C1=O] | [M + H]⁺ = 317 |
| 3-(4-((trifluoromethyl)thio)phenyl)-1,3- diazaspiro[4.4]nonane-2,4-dione | 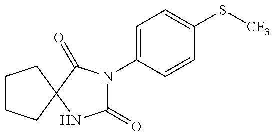 [SMILES: O=C1NC2(CCCC2)C(=O)N1c1ccc(SC(F)(F)F)cc1] | [M − H]⁻ = 329.1 |
| 3-(4-iodophenyl)-5,5-dimethylimidazolidine-2,4- dione | 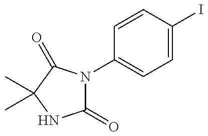 [SMILES: CC1(C)NC(=O)N(c2ccc(I)cc2)C1=O] | [M − H]⁻ = 329.0 |

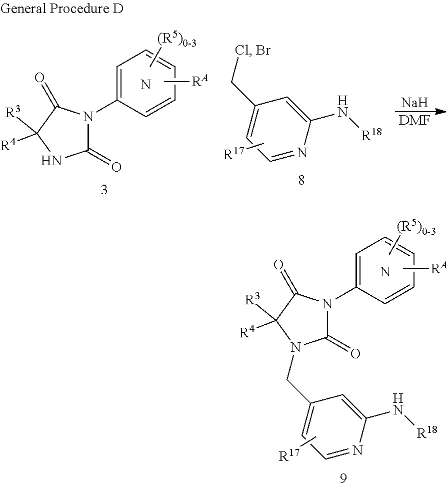

Hydantoin 3 (1 equiv.) was dissolved in DMF (0.1 M). Sodium hydride (3.5 equiv.) was added under N₂ and the resulting mixture was stirred at rt for 30 min. Then, a solution of benzyl halide 8 (1.1 equiv.) in DMF (0.5 M) was added dropwise. The resulting mixture was stirred at rt for 30 min, before carefully quenching the reaction with water. The reaction was partitioned between EtOAc and water. The aqueous layer was extracted with EtOAc (3×). The combined organic layers were washed with water, brine, and dried over Na₂SO₄. The crude mixture was purified by flash chromatography (SiO₂, EtOAc/heptanes) to afford product 9.

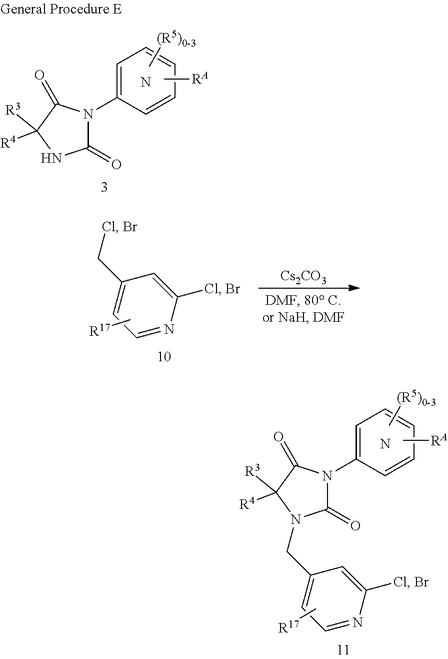

Hydantoin 3 (1 equiv.) was dissolved in DMF (0.3 M). Cesium carbonate (2.0 equiv.) was added, followed by benzyl halide 10 (1.1 equiv.). The resulting mixture was stirred at 80° C. for 1 h. After cooling to rt, the reaction was partitioned between EtOAc and water. The aqueous layer was extracted with EtOAc (2×). The combined organic layers were washed with water, brine, and dried over Na₂SO₄. The crude mixture was purified by flash chromatography (SiO₂, heptanes/EtOAc) to afford product 11.

Alternatively, hydantoin 3 (1 equiv.) was dissolved in DMF (0.1 M). Sodium hydride (3.5 equiv.) was added under N₂ and the resulting mixture was stirred at rt for 10 min. Then, a solution of benzyl halide 10 (1.1 equiv.) in DMF (0.5 M) was added dropwise. The resulting mixture was stirred at rt for 75 min, before carefully quenching the reaction with water. The reaction was partitioned between EtOAc and water. The aqueous layer was extracted with EtOAc (3×). The combined organic layers were washed with water, brine, and dried over Na₂SO₄. The crude mixture was purified by flash chromatography (SiO₂, EtOAc/heptanes) to afford product 11.

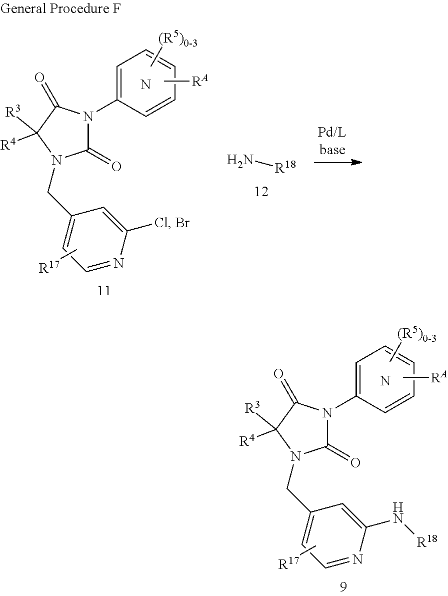

A mixture of an appropriately substituted halopyridine 11 (1 equiv.), amine 12 (1.2 equiv.), a palladium source chosen from Pd(OAc)₂ or Pd₂(dba)₃ (0.05 equiv Pd), a ligand chosen from BINAP, Xantphos or tBu-Josiphos (0.05 equiv.) and NaOtBu (1.4 equiv.) in a solvent chosen from dioxane, toluene or DME (0.1 M) was purged with N₂. The reaction mixture was heated at a temperature ranging from 70 to 100° C. until LC-MS analysis showed the reaction was complete. The reaction was diluted with EtOAc, washed with water and brine, and concentrated. The crude product was purified either by flash chromatography (SiO₂, eluting with heptanes/EtOAc) or by reversed phase flash chromatography (C18, eluting with 10 mM ammonium bicarbonate or ammonium formate (aq.) in MeCN) to afford the desired product 9.

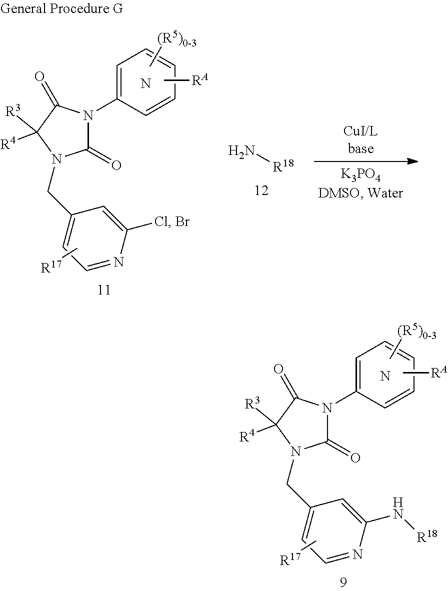

A mixture of an appropriately substituted halopyridine 11 (1 equiv.) and a primary amine 12 (3 equiv.), or its hydrochloride salt, were dissolved in DMSO (0.1 M) and water (10.1 ratio). The mixture was degassed for 5 minutes with nitrogen. Potassium phosphate tribasic (3 equiv for free amine base or 6 equiv. for hydrochloride salt), ligand [(2,6-dimethylphenyl)amino](oxo)acetic acid (0.6 equiv.) and copper iodide (0.25 equiv.) were added and the mixture was heated at 85° C. with stirring for 3-12 hours. The reaction mixture was cooled to RT, filtered, and concentrated in vacuo. The crude product was purified by reverse phase flash chromatography (C18, eluting with MeCN/10 mM ammonium bicarbonate (pH 10.3) or ammonium formate (pH 3.8) buffer) to afford the desired product 9.

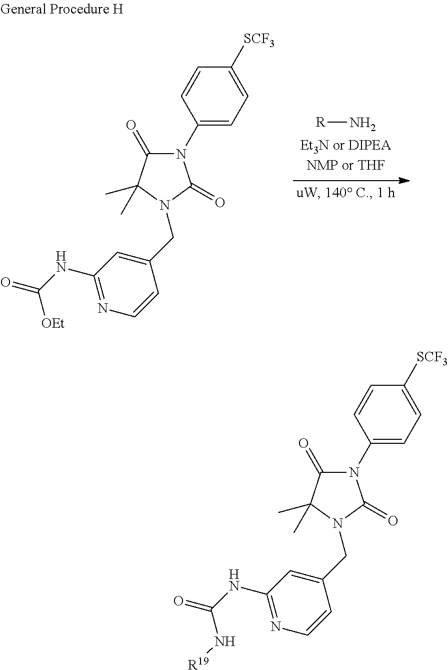

Ethyl(4-((5,5-dimethyl-2,4-dioxo-3-(4-((trifluoromethyl)thio)phenyl)imidazolidin-1-yl)methyl)pyridin-2-yl)carbamate (1 equiv.), an appropriately substituted amine (2-5 equiv.) and NMP (0.5-1 M) were heated in a microwave reactor at 140° C. for 1 h. The reaction was cooled to RT and diluted with EtOAc. The organic layer was washed with aqueous NH₄Cl, aqueous NaHCO₃, then brine. The organic layer was separated, dried over sodium sulfate, filtered, and concentrated in vacuo. The crude product was purified reverse phase flash chromatography (C₁₈, eluting with MeCN/10 mM ammonium bicarbonate (pH 10.3) or ammonium formate (pH 3.8) buffer) to afford the desired product.

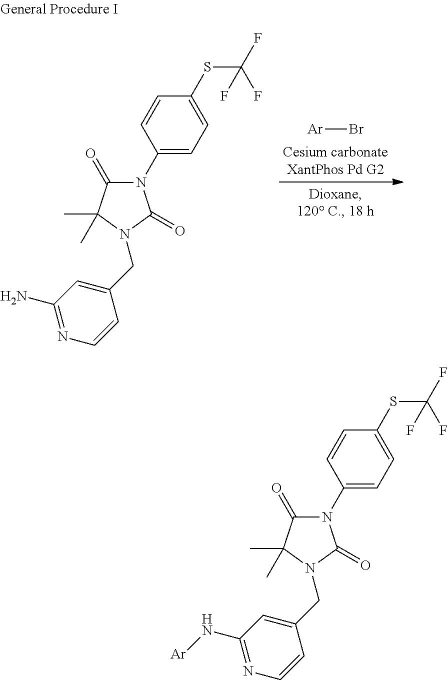

A mixture of 1-((2-aminopyridin-4-yl)methyl)-5,5-dimethyl-3-(4-((trifluoromethyl)thio)phenyl)imidazolidine-2,4-dione, corresponding aryl bromide (1-1.5 equiv.), cesium carbonate (3 equiv.) and dioxane (50-100 mM) was degassed. XantPhos Pd-G2 (0.1-0.15 equiv.) was added and reaction degassed. The reaction mixture was heated at 120° C. for 18 h with stirring. The reaction was cooled to rt and filtered through a plug of silica gel mixed with celite, cake was washed with EtOAc, then product eluted with 20% MeOH in DCM. The filtrate was concentrated in vacuo and purified by reverse phase flash chromatography (C18, eluting with MeCN/10 mM ammonium bicarbonate (pH 10.3) or ammonium formate (pH 3.8) buffer) to afford the desired product.

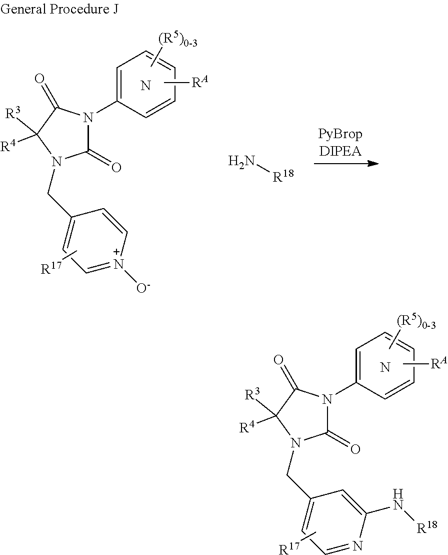

In a reaction vessel was combined pyridine-N-oxide (1 equiv), a primary amine (1.2 equiv), N,N-Diisopropylethylamine (5 equiv) and DCM (0.3 M). The solution was treated with PyBrop (1.3 equiv) at 0° C. The reaction was capped and stirred at room temperature overnight. Upon completion (LCMS analysis), the reaction was concentrated under reduced pressure. The crude was purified by column chromatography to give the desired product.

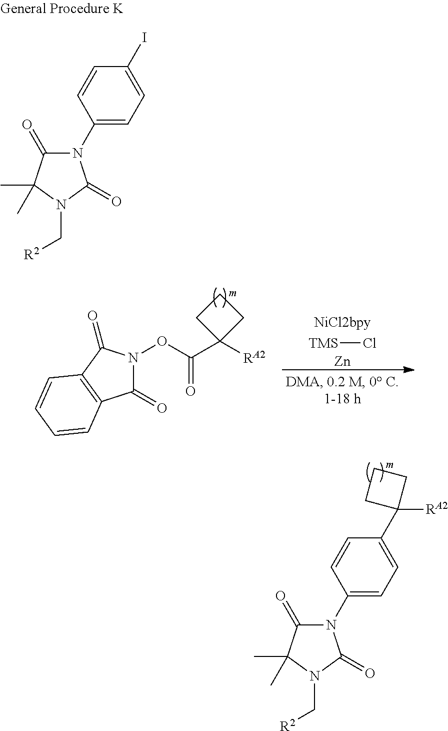

A reaction vessel containing Ar—I (1 equiv), NHP ester (1.2-2 equiv), NiCl₂bpy (15 mol %), and Zn (8 equiv) was evacuated and backfilled with N₂ (×3) before DMA (0.2 M) was added followed by TMS-Cl (3 equiv). The reaction vessel was quickly placed at 0° C. and stirred vigorous-y for 1-18 h. The reaction mixture was diluted with EtOAc and passed over a plug of silica gel or celite (eluting with EtOAc). The volatiles were removed under reduced pressure, and the crude material was purified by column chromatography to afford the desired product.

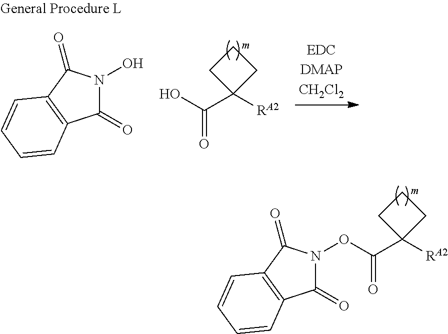

EDC (1.10 equivs) was added to a stirring solution of carboxylic acid (1.00 equivs), N-hydroxyphthalimide (1.05 equivs), and DMAP (0.200 equivs) at room temperature and stirred overnight. The reaction mixture was diluted with DCM and the organic layer was washed with 1 M HCl (×1), brine (×1), dried over MgSO₄, filtered, and concentrated under reduced pressure to afford the desired product which was of sufficient purity for use directly in the next step.

The following compounds are prepared according to General Procedure L.

| Name | Structure | Characterization Data |
|---|---|---|
| 1,3-dioxoisoindolin-2-yl spiro[2.2]pentane-1- carboxylate | 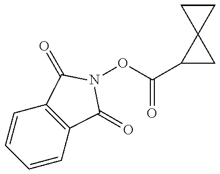 [SMILES: O=C(ON1C(=O)c2ccccc2C1=O)C1CC12CC2] | ¹H NMR (400 MHz, CDCl₃) δ (ppm): 7.98-7.83 (m, 2H), 7.81- 7.66 (m, 2H), 2.31 (dd, J = 7.5, 4.1 Hz, 1H), 1.76 (t, J = 4.1 Hz, 1H), 1.68 (dd, J = 7.5, 4.1 Hz, 1H), 1.16-0.98 (m, 4H). |
| 1,3-dioxoisoindolin-2-yl 1- methylcyclopropane-1- carboxylate | 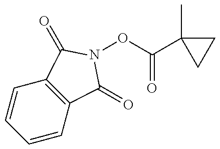 [SMILES: CC1(C(=O)ON2C(=O)c3ccccc3C2=O)CC1] | 1H NMR (400 MHz, CDCl3) δ 7.91-7.85 (m, 2H), 7.81-7.75 (m, 2H), 1.59-1.54 (m, 2H), 1.48 (s, 3H), 0.99-0.94 (m, 2H). |
| 1,3-dioxoisoindolin-2-yl 1- methoxycyclopropane-1- carboxylate | 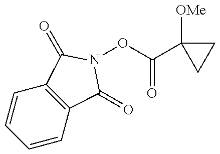 [SMILES: COC1(C(=O)ON2C(=O)c3ccccc3C2=O)CC1] | ¹H NMR (400 MHz, CDCl₃) δ (ppm): 7.93-7.85 (m, 2H), 7.84- 7.76 (m, 2H), 3.55 (s, 3H), 1.59 (dd, J = 8.5, 5.1 Hz, 2H), 1.42 (dd, J = 8.5, 5.1 Hz, 2H). |
| 1,3-dioxoisoindolin-2-yl 1- cyanocyclobutane-1- carboxylate | 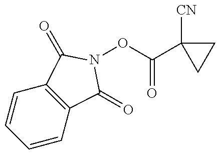 [SMILES: N#CC1(C(=O)ON2C(=O)c3ccccc3C2=O)CC1] | ¹H NMR (400 MHz, CDCl₃) δ (ppm): 7.95-7.89 (m, 2H), 7.85- 7.80 (m, 2H), 3.02-2.92 (m, 2H), 2.91-2.82 (m, 2H), 2.45- 2.27 (m, 2H). |
| 1,3-dioxoisoindolin-2-yl 2,2- difluoro-1- methylcyclopropane-1- carboxylate | 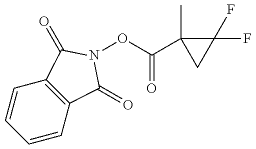 [SMILES: CC1(C(=O)ON2C(=O)c3ccccc3C2=O)CC1(F)F] | 1H NMR (400 MHz, CDCl3) δ 7.93-7.86 (m, 2H), 7.83-7.77 (m, 2H), 2.42 (ddd, J = 12.1, 8.0, 7.1 Hz, 1H), 1.66 (t, J = 2.3 Hz, 3H), 1.60 (ddd, J = 10.5, 8.2, 6.0 Hz, 1H). |
| 1,3-dioxoisoindolin-2-yl 1- (trifluoromethyl)cyclobutane- 1-carboxylate | 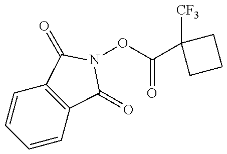 [SMILES: O=C1c2ccccc2C(=O)N1OC(=O)C1(C(F)(F)F)CCC1] | ¹H NMR (400 MHz, CDCl₃) δ (ppm): 7.94-7.89 (m, 2H), 7.84- 7.79 (m, 2H), 2.92-2.79 (m, 2H), 2.73-2.62 (m, 2H), 2.29- 2.14 (m, 2H). |
| 1,3-dioxoisoindolin-2-yl bicyclo[1.1.1]pentane-1- carboxylate | 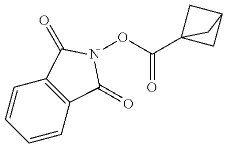 [SMILES: O=C1c2ccccc2C(=O)N1OC(=O)C12CC(C1)C2] | ¹H NMR (400 MHz, CDCl₃) δ (ppm): 7.91-7.85 (m, 2H), 7.81- 7.76 (m, 2H), 2.55 (s, 1H), 2.33 (s, 6H). |

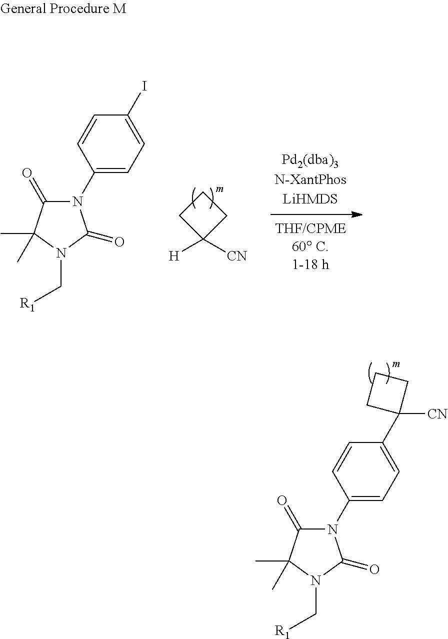

Bis(diphenylphosphino)phenoxazine (20 mol %), and evacuated and backfilled with N₂ (×3), and then freshly degassed (sparged with N₂ for 15 minutes) THF (175 mM) was added under an inert atmosphere and was stirred for 15 minutes at room temperature. Then, solid Ar—I (1.0 equivs) was quickly added, followed by the nitrile (2 equivs), freshly degassed (sparged with N₂ for 15 min) CPME (175 mM) and LiHMDS (I M in THF, 3.6 equivs), and the reaction was placed in a pre-heated oil bath at 60° C. for 1-18 h. The reaction mixture was cooled to ambient temperature before MeOH was added and stirred for 15 min. The volatiles were removed under reduced pressure, and the obtained crude product was purified by column chromatography to afford the desired product.

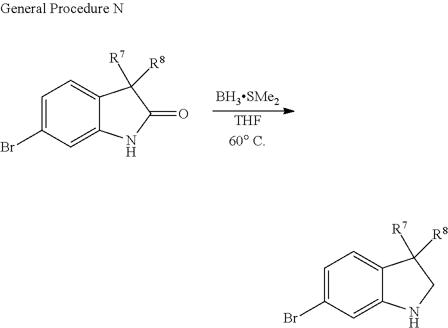

To a solution of the substituted indolone (1 equiv.) in THF (0.25 M) at 0° C. was added BH₃·SMe₂ (4 equiv.) dropwise. The reaction mixture was then refluxed at 60° C. for 16 h. The reaction mixture was quenched with MeOH and then concentrated to dryness. This process was repeated 5 times. The crude product was purified by normal phase chromatography (SiO₂, eluting with Hexanes/EtOAc) to afford the desired product.

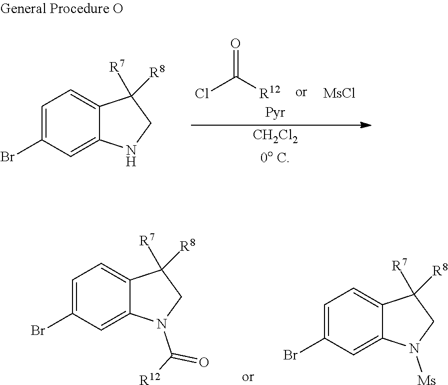

To a solution of the substituted indoline (1 equiv.) in CH₂Cl₂ (0.25 M) at 0° C. was added pyridine (2 equiv.) and the appropriate acyl chloride or MsCl (1.1 equiv.). The reaction mixture was stirred at 0° C. for 1 h, then RT for 1 h. The reaction mixture was quenched with water and extracted with CH₂Cl₂. The combined organics was washed with water, brine, dried over anhydrous Na₂SO₄, and concentrated to dryness. The crude product was purified by normal phase chromatography (SiO₂, eluting with Hexanes/EtOAc) to afford the desired product.

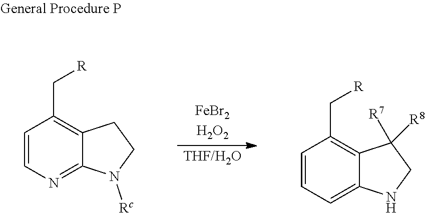

To a solution of the substituted aza-indole (1 equiv.) in THF (0.2 M) and H₂O (2 M) was added FeBr₂ (0.1 equiv.). H₂O₂ (4.4 equiv., 30% in water) was added and the reaction mixture was stirred for 1 h. The reaction mixture was quenched with sat. Na₂S₂O₃ and extracted with EtOAc. The combined organics was washed with water, brine, dried over anhydrous Na₂SO₄, and concentrated to dryness. The crude product was purified by reverse phase chromatography to afford the desired product.

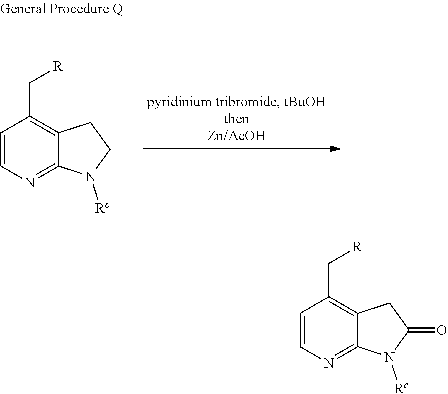

A flask containing aza-indole (1 equivs), pyridinium tribromide (6 equivs), and tBuOH (55 mM) were stirred at 35 C for 1 h. The reaction was diluted with EtOAc and the organic layer was washed with saturated ammonium chloride (×1), brine (×1), dried over MgSO₄, filtered, and concentrated under reduced pressure to afford a solid. This solid was suspended in AcOH (45 mM) and Zinc (6 equivs) was added. The reaction was stirred at room temperature for 30 min, before being diluted with EtOAc, and filtered over a short pad of celite (washing with EtOAc). The volatiles were removed under reduced pressure and dried under vacuum overnight. The crude material was purified by reverse phase column chromatography on C18 to afford the product.

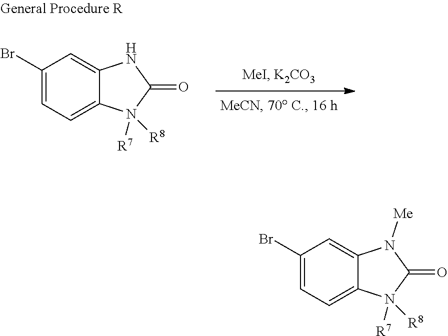

In a sealable reaction vessel was added the substituted 6-bromoindolin-2-one (or 6-bromoindoline) (1.0 equiv) and potassium carbonate (3.0 equiv) and dissolved in MeCN (0.25 M). The reaction vessel was sealed and purged with an atmosphere of nitrogen. Iodomethane (2.5 equiv) was added dropwise and the reaction was heated to 70° C. for 16 h. The reaction mixture was diluted with water and extracted with EtOAc (3×). The combined organic layers were dried with Na₂SO₄ and concentrated onto silica. The reaction was purified by flash column chromatography on silica gel column, eluting with a solvent gradient of 0-30% EtOAc/hexanes to afford the N-methylated product.

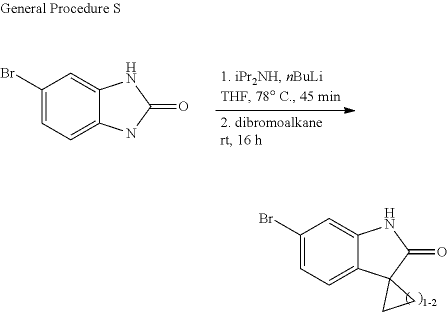

n-Butyllithium (3.0 equiv) was added dropwise at −78° C. to a stirring solution of 6-bromoindolin-2-one (1.0 equiv) and diisopropylamine (3.0 equiv) in THF (0.2 M). The mixture was stirred for 45 min while maintaining this temperature, before adding either 1,2-dibromoethane or 1,3-dibromopropane (3.0 equiv) dropwise. Stirring was continued for 16 h while the temperature was allowed to slowly raised to room temperature. The reaction mixture was quenched with 4 N HCl and the aqueous portion was separated and extracted further with EtOAc (3×). The combined organic layers were washed with water, and brine before drying over Na₂SO₄. The material was concentrated on silica for purification. The crude reaction was purified by flash column chromatography on silica gel column, eluting with a solvent gradient of 0-40% EtOAc/hexanes to afford the spirocyclic product.

### Example A-1: Preparation of 5,5-dimethyl-3-(1′-methylspiro[cyclobutane-1,3′-indolin]-6′-yl)imidazolidine-2,4-dione

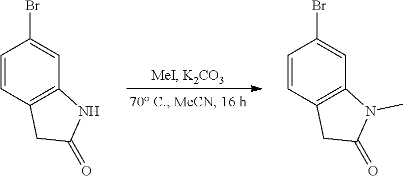

Step 1: 6-bromo-1-methylindolin-2-one: The title compound was prepared from 6-bromoindolin-2-one according to General Procedure R. MS (m/z): [M+H]⁺: 226.1.

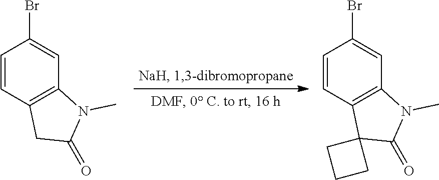

Step 2: 6′-bromo-1′-methylspiro[cyclobutane-1,3′-indolin]-2′-one: 6-Bromo-1-methylindolin-2-one (323 mg, 1.43 mmol) was dissolved in DMF (25.0 mL) under an atmosphere of nitrogen. The reaction was cooled to 0° C. and sodium hydride (60% in dispersion in mineral oil) (98 mg, 4.09 mmol) was added and reaction stirred for 30 min while warming to room temperature, 1,3-dibromopropane (417 mg, 2.04 mmol) was added dropwise and the reaction was left to stir for 16 h. The crude material was purified by flash column chromatography (SiO₂, 0-20% EtOAc/hexanes) to deliver 6′-bromo-F′-methylspiro[cyclobutane-1,3′-indolin]-2′-one (191 mg, 35%). MS (m/z): [M+H]⁺: 266.1.

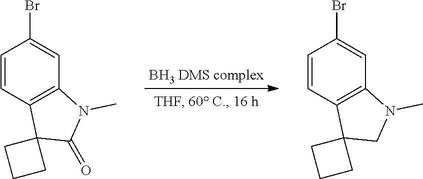

Step 3: 6′-bromo-F′-methylspiro[cyclobutane-1,3′-indoline]: The title compound was prepared from 6′-bromo-1′-methylspiro[cyclobutane-1,3′-indolin]-2′-one according to General Procedure N. MS (m/z): [M+H]⁺; 252.1, 254.1.

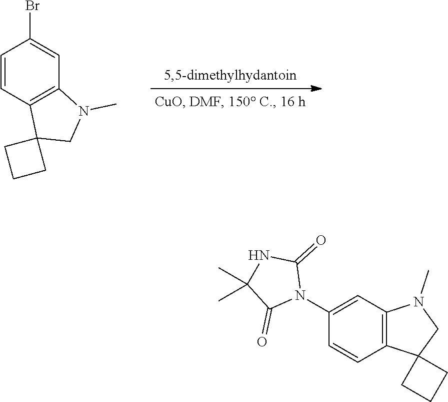 [SMILES: CN1CC2(CCC2)c2ccc(Br)cc21 ; CN1CC2(CCC2)c2ccc(N3C(=O)NC(C)(C)C3=O)cc21]

Step 4: 5.5-dimethyl-3-(1′-methylspiro[cyclobutane-1,3′-indolin]-6′-yl)imidazolidine-2,4-dione: The title compound was prepared from 6′-bromo-1′-methylspiro[cyclobutane-1,3′-indoline]according to General Procedure A. MS (m/z): [M+H]⁺: 300.2.

### Example A-2: Preparation of 3-(3,3-dimethyl-1-(methylsulfonyl)indolin-6-yl)-5,5-dimethylimidazolidine-2,4-dione

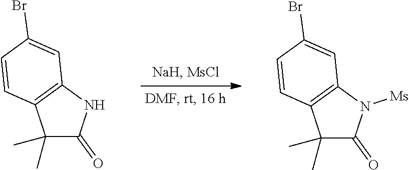 [SMILES: CC1(C)C(=O)Nc2cc(Br)ccc21 ; CC1(C)C(=O)N(S(C)(=O)=O)c2cc(Br)ccc21]

Step 1: 6-bromo-3,3-dimethyl-1-(methylsulfonyl)indolin-2-one: A flask was charged with 6-bromo-3.3-dimethylindolin-2-one (300 mg, 1.21 mmol) followed by DMF (5.8 mL, 0.2 M), and the suspension cooled to 0° C. Sodium hydride (60% in dispersion in mineral oil) (58 mg, 2.42 mmol) was added, and the reaction was stirred for 5 min before the addition of methanesulfonyl chloride (188 μL, 2.42 mmol). The reaction was warmed to room temperature over 16 h, at which point the reaction was quenched with water and extracted with EtOAc (3×). The combined organic layers were washed with brine, dried over Na₂SO₄, filtered, and concentrated. The crude reaction was purified by reverse phase (C₁₈ column, 35-55% MeCN/0.1% of 10 mM ammonium formate buffer) to afford the 6-bromo-3,3-dimethyl-1-(methylsulfonyl)indolin-2-one (258 mg, 67%). MS (m/z): [M+H]⁺: 319.9.

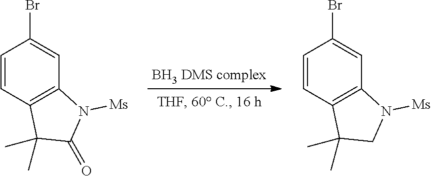 [SMILES: CC1(C)C(=O)N(S(C)(=O)=O)c2cc(Br)ccc21 ; CC1(C)CN(S(C)(=O)=O)c2cc(Br)ccc21]

Step 2: 6-bromo-3.3-dimethyl-1-(methylsulfonyl)indoline: The title compound was prepared from 6-bromo-3,3-dimethyl-1-(methylsulfonyl)indolin-2-one according to General Procedure N. MS (m/z): [M+H]⁺: 304.2.

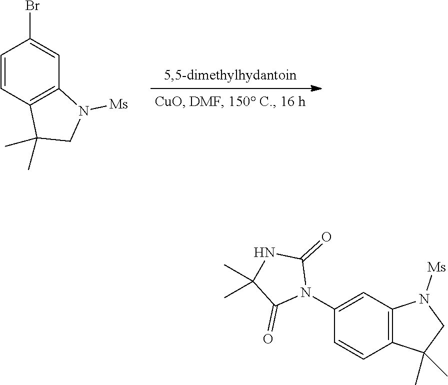

Step 3: 3-(3,3-dimethyl-1-(methylsulfonyl)indolin-6-yl)-5.5-dimethylimidazolidine-2.4-dione: The title compound was prepared from 6-bromo-3,3-dimethyl-1-(methylsulfonyl)indoline according to General Procedure A. MS (m/z): [M+H]⁺: 352.2.

### Example A-3: Preparation of 1-(4-(4,4-dimethyl-2,5-dioxoimidazolidin-1-yl)phenyl)cyclobutane-1-carbonitrile

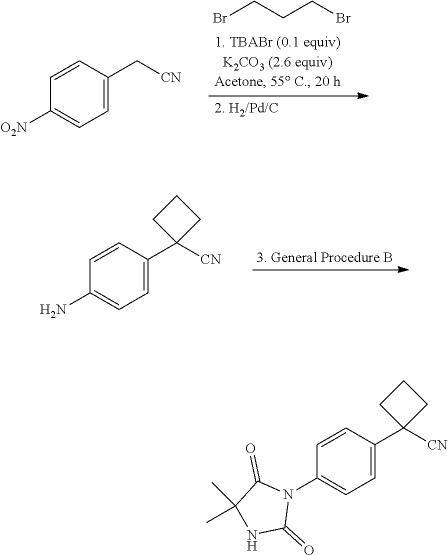 [SMILES: BrCCCBr ; N#CCc1ccc([N+](=O)[O-])cc1 ; N#CC1(c2ccc(N)cc2)CCC1 ; CC1(C)NC(=O)N(c2ccc(C3(C#N)CCC3)cc2)C1=O]

Step 1: Acetone (100 mL) was added to 4-nitrophenylacetonitrile (5.00 g, 30.2 mmol), potassium carbonate (10.9 g, 78.6 mmol), and tetrabutylammonium bromide (974 mg, 3.02 mmol), 1,3-Dibromopropane (3.73 mL, 36.3 mmol) was then added and the reaction was heated 55° C. and left to stir for 20 h. The reaction was cooled to rt and the precipitate was filtered over celite, eluting with acetone, and the filtrate concentrated under reduced pressure. The crude material was purified by column chromatography (SiO₂, 0-50% EtOAc/heptanes) to afford the product (3.00 g, 49%). ¹H NMR (400 MHz, CDCl₃) δ (ppm): 8.30-8.23 (m, 2H), 7.64-7.56 (m, 2H), 2.95-2.85 (m, 2H), 2.71-2.59 (m, 2H), 2.57-2.45 (m, 1H), 2.20-2.07 (m, 1H).

Step 2: The material from the previous step (1.00 g, 4.95 mmol) was hydrogenated with 10% Pd/C (100 mg, 0.940 mmol) under an atmosphere of H₂ in MeOH (50.0 mL) to afford the product (0.763 g, 87%).

Step 3: The title compound was synthesized according to General Procedure B using the material from the previous step (763 mg, 4.43 mmol), toluene (44.3 mL), trichloromethyl chloroformate (428 μL, 3.54 mmol), triethylamine (2.56 mL, 18.2 mmol), and—ethyl α—aminoisobutyrate hydrochloride (730 mg, 4.61 mmol). The crude material was purified by column chromatography (SiO₂, 0-100% EtOAc/heptanes) to afford the title compound (721 mg, 57%). LCMS: MS (m/z): [M−H]⁻=282.2.

### Example 1: Preparation of 1-((2-aminopyridin-4-yl)methyl)-3-(4-(tert-butyl)phenyl)-5,5-dimethylimidazolidine-2,4-dione

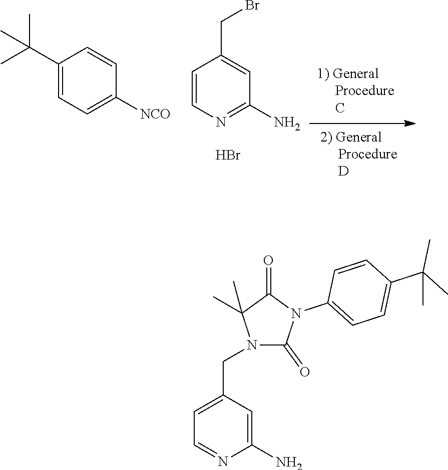

Step 1: Hydantoin core was prepared according to the General Procedure C with 4-tert-butylphenyl isocyanate and Methyl α-aminoisobutyrate hydrochloride. The crude material was carried forward as is.

Step 2: The title compound was prepared according to the General Procedure D with the hydrobromide salt of 4-(bromomethyl)pyridin-2-amine. MS (m/z): [M+H]⁺=367.6, ¹H NMR (400 MHz, DMSO) δ (ppm): 7.82 (d, J=5.2 Hz, 1H), 7.55-7.45 (m, 2H), 7.37-7.28 (m, 2H), 6.46 (dd, J=5.3, 1.4 Hz, 1H), 6.40 (s, 1H), 5.87 (s, 2H), 4.41 (s, 2H), 1.36 (s, 6H), 1.29 (s, 9H).

### Example 2: Preparation of 1-((1H-pyrrolo[2,3-b]pyridin-4-yl)methyl)-5,5-dimethyl-3-(4-(1-(trifluoromethyl)cyclopropyl)phenyl)imidazolidine-2,4-dione (Compound 7)

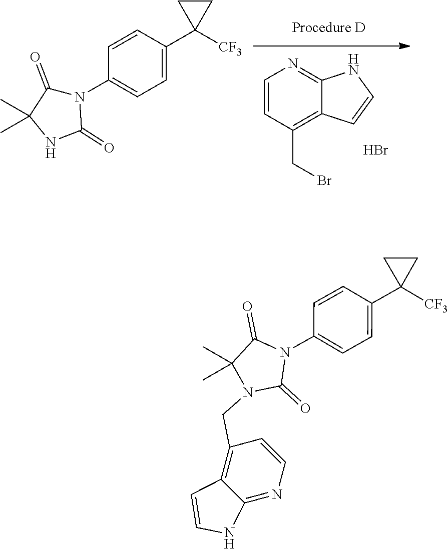 [SMILES: CC1(C)NC(=O)N(c2ccc(C3(C(F)(F)F)CC3)cc2)C1=O ; BrCc1ccnc2[nH]ccc12 ; Br ; CC1(C)C(=O)N(c2ccc(C3(C(F)(F)F)CC3)cc2)C(=O)N1Cc1ccnc2[nH]ccc12]

The title compound was prepared according to the General Procedure D using 4-(bromomethyl)-1H-pyrrolo[2,3-b]pyridine hydrobromide. MS (m/z): [M+H]⁺=443.2, ¹H NMR (400 MHz, DMSO) δ (ppm): 11.69 (s, 1H), 8.16 (d, J=4.9 Hz, 1H), 7.58 (d, J=8.4 Hz, 2H), 7.52-7.42 (m, 3H), 7.10 (d, J=4.9 Hz, 1H), 6.62 (d, J=3.4 Hz, 1H), 4.86 (s, 2H), 1.39-1.30 (m, 8H), 1.19-1.14 (m, 2H).

### Example 3: Preparation of 1-((3-bromo-1H-pyrrolo[2,3-b]pyridin-4-yl)methyl)-5,5-dimethyl-3-(4-(1-(trifluoromethyl)cyclopropyl)phenyl)imidazolidine-2,4-dione (Compound 8)

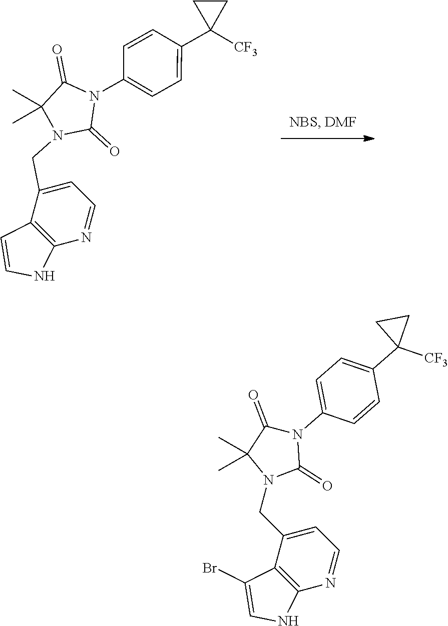 [SMILES: CC1(C)C(=O)N(c2ccc(C3(C(F)(F)F)CC3)cc2)C(=O)N1Cc1ccnc2[nH]ccc12 ; CC1(C)C(=O)N(c2ccc(C3(C(F)(F)F)CC3)cc2)C(=O)N1Cc1ccnc2[nH]cc(Br)c12]

1-((1H-pyrrolo[2,3-b]pyridin-4-yl)methy)-5,5-dimethyl-3-(4-(1-(trifluoromethyl)cyclopropyl)phenyl)imidazolidine-2,4-dione (from Example 2) and NBS (20.1 mg, 113 μmol) were stirred in DMF (1.70 mL) for 2 h. The mixture was filtered and purified by reverse phase (C18, 10 mM Ammonium Bicarbonate Buffer pH 10.3/MeCN, 30%-50%) to afford the title compound (6.1 mg, 10%) MS (m/z): [M+H]⁺=521.1/523.0. ¹H NMR (400 MHz, DMSO) δ (ppm): 8.21 (d, J=4.9 Hz, 1H), 7.71 (s, 1H), 7.58 (d, J=8.4 Hz, 2H), 7.49 (d, J=8.5 Hz, 2H), 7.15 (d, J=4.9 Hz, 1H), 5.21 (s, 2H), 1.42 (s, 6H), 1.38-1.31 (m, 2H), 1.20-1.13 (m, 2H).

### Example 4: Preparation of 5,5-dimethyl-1-((2-oxo-2,3-dihydro-1H-pyrrolo[2,3-b]pyridin-4-yl)methyl)-3-(4-(1-(trifluoromethyl)cyclopropyl)phenyl)imidazolidine-2,4-dione (Compound 13)

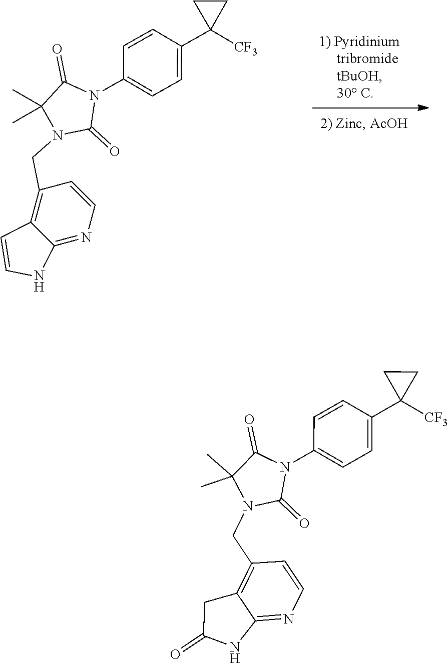 [SMILES: CC1(C)C(=O)N(c2ccc(C3(C(F)(F)F)CC3)cc2)C(=O)N1Cc1ccnc2[nH]ccc12 ; CC1(C)C(=O)N(c2ccc(C3(C(F)(F)F)CC3)cc2)C(=O)N1Cc1ccnc2c1CC(=O)N2]

The compound was prepared by following General Procedure Q.

Step 1: 1-((1H-pyrrolo[2,3-b]pyridin-4-yl)methyl)-5,5-dimethyl-3-(4-(1-(trifluoromethyl)cyclopropyl)phenyl)imidazolidine-2,4-dione (110 mg, 69.1 μmol), pyridinium tribromide (353 mg, 994 μmol) and tBuOH (5.4 mL) were stirred at 30° C. for 1 h. The reaction was diluted with EtOAc and washed with saturated ammonium chloride and brine, dried over sodium sulfate, filtered, and concentrated in vacuo affording a brown semi-solid carried forward as-is.

Step 2: The residue from step 1 was taken up in AcOH (10 mL) and Zinc dust (163 mg, 2.49 mmol) was added. The reaction was stirred at room temperature for 30 min. The mixture was filtered and the AcOH removed in vacuo. The residue was taken up in EtOAc and added to saturated sodium bicarbonate. The aqueous was extracted with EtOAc, the combined organics were dried over sodium sulfate, filtered, and concentrated in vacuo. The crude product was purified by reverse phase (C18, 10 mM Ammonium Formate Buffer pH 3.8/MeCN, 25%-45%) to afford the title compound (10.0 mg, 40%). MS (m/z): [M+H]⁺=459.2, ¹H NMR (400 MHz, DMSO) δ (ppm): 10.99 (s, 1H), 8.00 (d, J=5.5 Hz, 1H), 7.57 (d, J=8.4 Hz, 2H), 7.51-7.42 (m, 2H), 6.96 (d, 3=5.5 Hz, 1H), 4.51 (s, 2H), 3.62 (s, 2H), 1.41-1.33 (m, 8H), 1.15 (d, J=5.5 Hz, 2H).

### Example 5: Preparation of 1-((2,3-dihydro-1H-pyrrolo[2,3-b]pyridin-4-yl)methyl)-5,5-dimethyl-3-(4-((trifluoromethyl)thio)phenyl)imidazolidine-2,4-dione (Compound 16)

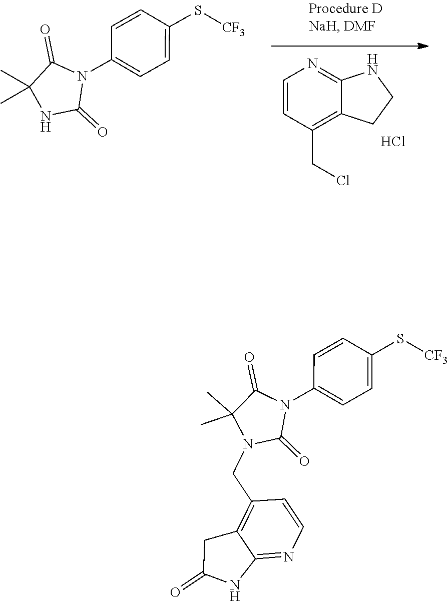 [SMILES: CC1(C)NC(=O)N(c2ccc(SC(F)(F)F)cc2)C1=O ; ClCc1ccnc2c1CCN2 ; Cl ; CC1(C)C(=O)N(c2ccc(SC(F)(F)F)cc2)C(=O)N1Cc1ccnc2c1CC(=O)N2]

The title compound was prepared according to the General Procedure D using 5,5-dimethyl-3-(4-((trifluoromethyl)thio)phenyl)imidazolidine-2,4-dione and 4-(chloromethyl)-2,3-dihydro-1H-pyrrolo[2,3-b]pyridine hydrochloride. MS (m/z). [M+H]⁺=437.2, ¹H NMR (400 MHz, DMSO) δ (ppm): 7.89-7.80 (m, 2H), 7.69-7.60 (m, 3H), 6.43 (d, J=5.5 Hz, 1H), 6.33 (s, 1H), 4.40 (s, 2H), 3.46 (t, J=8.6 Hz, 2H), 3.01 (t, J=8.4 Hz, 2H), 1.38 (s, 6H).

### Example 6: Preparation of 5,5-dimethyl-1-((2-oxo-2,3-dihydro-1H-pyrrolo[2,3-b]pyridin-4-yl)methyl)-3-(4-((trifluoromethyl)sulfonyl)phenyl)imidazolidine-2,4-dione (Compound 23)

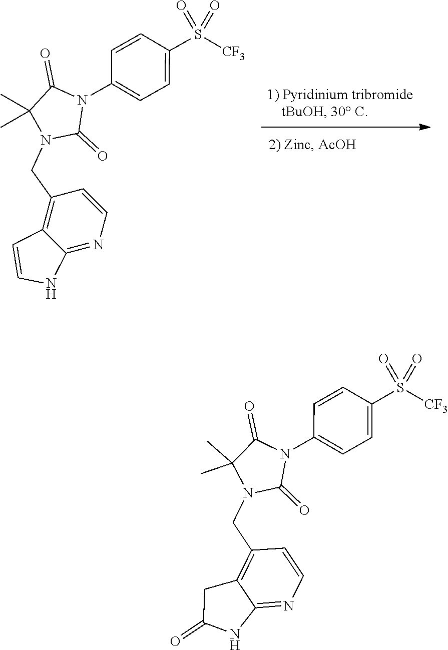 [SMILES: CC1(C)C(=O)N(c2ccc(S(=O)(=O)C(F)(F)F)cc2)C(=O)N1Cc1ccnc2[nH]ccc12 ; CC1(C)C(=O)N(c2ccc(S(=O)(=O)C(F)(F)F)cc2)C(=O)N1Cc1ccnc2c1CC(=O)N2]

The title compound was prepared according to the procedures of Example 4 (General Procedure Q) (15.0 mg, 29%). MS (m/z): [M+H]⁺=483.1. ¹H NMR (400 MHz, DMSO) δ (ppm): 11.00 (s, 1H), 8.30 (d, J=8.8 Hz, 2H), 8.07-8.01 (m, 2H), 8.00 (d, J=5.5 Hz, 1H), 7.03 (d, J=5.5 Hz, 1H), 4.54 (s, 2H), 3.65 (s, 2H), 1.40 (s, 6H).

### Example 7: Preparation of 2-(4-(4,4-dimethyl-2,5-dioxo-3-((2-oxo-2,3-dihydro-1H-pyrrolo[2,3-b]pyridin-4-yl)methyl)imidazolidin-1-yl)phenyl)-2-methylpropanenitrile (Compound 29)

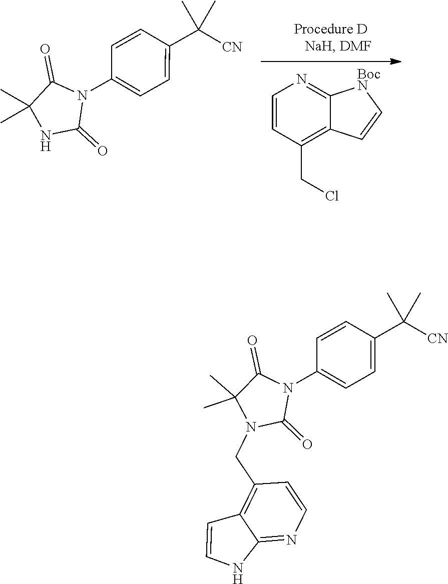 [SMILES: CC1(C)NC(=O)N(c2ccc(C(C)(C)C#N)cc2)C1=O ; CC(C)(C)OC(=O)n1ccc2c(CCl)ccnc21 ; CC(C)(C#N)c1ccc(N2C(=O)N(Cc3ccnc4[nH]ccc34)C(C)(C)C2=O)cc1]

Step 1: 2-(4-(3-((1H-pyrrolo[2,3-b]pyridin-4-yl)methyl)-4,4-dimethyl-2,5-dioxoimidazolidin-1-yl)phenyl)-2-methylpropanenitrile: The title compound was prepared according to the General Procedure D using 2-(4-(4,4-dimethyl-2,5-dioxoimidazolidin-1-yl)phenyl)-2-methylpropanenitrile and tert-butyl 4-(chloromethyl)-1H-pyrrolo[2,3-b]pyridine-1-carboxylate. The Boc group was removed using 4 M HCl in dioxane solution (15 eq.) MS (m/z): [M+H]⁺=402.

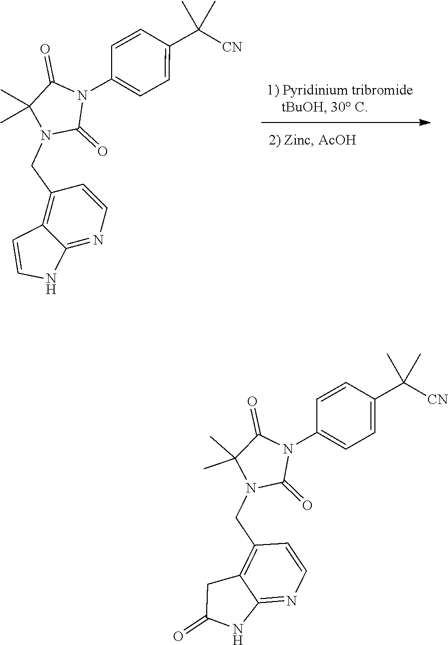 [SMILES: CC(C)(C#N)c1ccc(N2C(=O)N(Cc3ccnc4[nH]ccc34)C(C)(C)C2=O)cc1 ; CC(C)(C#N)c1ccc(N2C(=O)N(Cc3ccnc4c3CC(=O)N4)C(C)(C)C2=O)cc1]

Step 2: The title compound was prepared according to the procedures of Example 4 (General Procedure Q). MS (m/z): [M+H]⁺=418.4. ¹H NMR (400 MHz, DMSO) δ (ppm): 11.00 (s, 1H), 8.01 (d, J=5.5 Hz, 1H), 7.67-7.61 (m, 2H), 7.53-7.47 (m, 2H), 6.96 (d, J=5.5 Hz, 1H), 4.51 (s, 2H), 3.63 (s, 2H), 1.70 (s, 6H), 1.38 (s, 6H).

### Example 8: Preparation of 5,5-dimethyl-1-((2-oxo-2,3-dihydro-1H-pyrrolo[2,3-b]pyridin-4-yl)methyl)-3-(4-(1-(trifluoromethyl)cyclobutyl)phenyl)imidazolidine-2,4-dione hydrochloride (Compound 30)

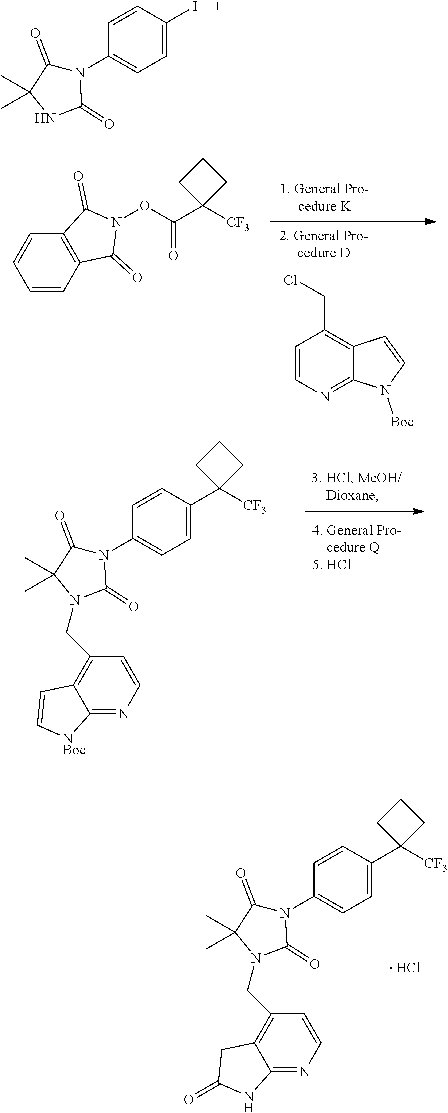

Step 1: The intermediate dione is prepared according to General Procedure K using zinc (1.27 g, 19.4 mmol), NiCl₂bpy (104 mg, 0.364 mmol), 3-(4-iodophenyl)-5,5-dimethylimidazolidine-2,4-dione, (800 mg, 2.42 mmol), and 1,3-dioxoisoindolin-2-yl 1-(trifluoromethyl)cyclobutane-1-carboxylate (1.14 g, 3.64 mmol), DMA (12.0 mL), chlorotrimethylsilane (942 μL, 7.27 mmol). The crude material was purified by column chromatography (SiO₂, 0-100% EtOAc/Heptanes) to afford the product as an oil (629 mg, 64% yield). LCMS MS (m/z): [M−H]⁻=325.1 ¹H NMR (400 MHz, CDCl₃) δ (ppm): 7.44-7.32 (m, 4H), 6.48-6.13 (m, 11H), 2.81-2.70 (m, 2H), 2.62-2.47 (m, 2H), 2.25-2.12 (m, 1H), 1.97-1.84 (m, 1H), 1.52 (s, 6H).

Step 2: tert-butyl 4-((5,5-dimethyl-2,4-dioxo-3-(4-(1-(trifluoromethyl)cyclobutyl)phenyl)imidazolidin-1-yl)methyl)-1H-pyrrolo[2,3-b]pyridine-1-carboxylate: Intermediate compound is prepared according to General Procedure D using the material from the previous step (629 mg, 1.93 mmol), tert-butyl 4-(chloromethyl)-1H-pyrrolo[2,3-b]pyridine-1-carboxylate (514 mg, 1.93 mmol), cesium carbonate (1.92 g, 5.78 mmol), DMF (5.00 mL) to afford intermediate (1.00 g, 93%). LCMS MS (m/z): [M+H-Boc]⁺=457.2.

Step 3: Intermediate compound from step 3 (1.00 g, 1.8 mmol) was dissolved in MeOH (3.00 mL) and hydrochloric acid (4.50 mL, 18.0 mmol, 4 M in dioxanes) was added. The reaction was stirred overnight at rt, then heated at 40° C. for 2 hours. The volatiles were removed under reduced pressure to afford the product (822 mg, >99% yield).

Step 4: The final compound was synthesized according to the procedures of Example 4 (General Procedure Q) using the material from the previous step (822 mg, 1.80 mmol), pyridinium tribromide (3.84 g, 10.8 mmol), tBuOH (18.0 mL) followed by AcOH (18.0 mL), zinc (707 mg, 10.8 mmol). The crude material was purified by column chromatography (SiO₂, 0-100% (2.5% NH₄OH, 25% MeOH in EtOAc)/EtOAc) then reverse phase chromatography (C18, 10-100% MeCN/0.1% of 10 mM ammonium bicarbonate buffer). Crude material was then dissolved in MeOH/DCM and 2 equiv of HCl (1 M) was added. The volatiles were removed under reduced pressure and the residue was dried to afford the title compound (251 mg, 27%). LCMS MS (m/z): [M+H; freebase]⁺=473.3. ¹H NMR (400 MHz, DMSO) δ (ppm): 11.08 (s, 1H), 8.01 (d, J=5.6 Hz, 1H), 7.53-7.40 (m, 4H), 6.98 (d, J=5.6 Hz, 1H), 5.74 (s, 1H), 4.53 (s, 2H), 3.64 (s, 2H), 2.75-2.52 (m, 4H), 2.15-1.99 (m, 1H), 1.96-1.81 (m, 1H), 1.38 (s, 6H). ¹H NMR data for free base: 1H NMR (400 MHz, DMSO) δ (ppm): 11.00 (brs, 1H), 8.00 (d, J=5.5 Hz, 1H), 7.52-7.31 (m, 4H), 6.96 (d, J=5.5 Hz, 1H), 4.52 (s, 2H), 3.63 (s, 2H), 2.73-2.64 (m, 2H), 2.64-2.52 (m, 2H), 2.15-2.02 (m, 1H), 1.96-1.84 (m, 1H), 1.38 (s, 6H).

### Example 9: Preparation of (1-(4-(4,4-dimethyl-2.5-dioxo-3-((2-oxo-2,3-dihydro-1H-pyrrolo[2,3-b]pyridin-4-yl)methyl)imidazolidin-1-yl)phenyl)cyclobutane-1-carbonitrile) (Compound 40)

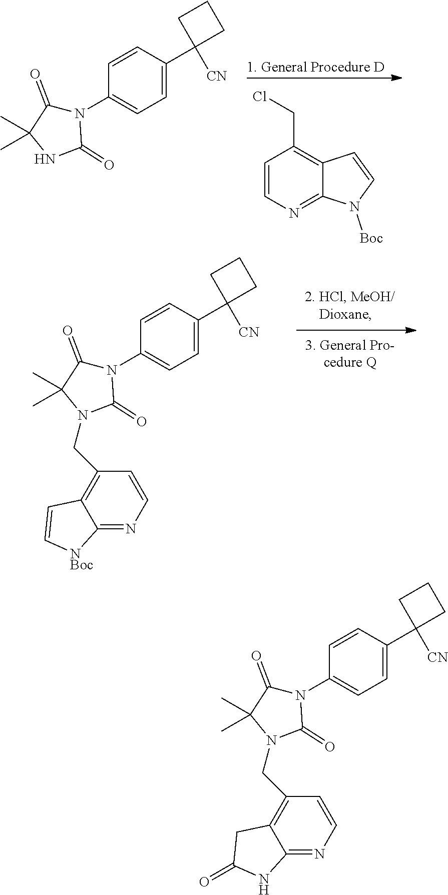 [SMILES: CC1(C)NC(=O)N(c2ccc(C3(C#N)CCC3)cc2)C1=O ; CC(C)(C)OC(=O)n1ccc2c(CCl)ccnc21 ; CC(C)(C)OC(=O)n1ccc2c(CN3C(=O)N(c4ccc(C5(C#N)CCC5)cc4)C(=O)C3(C)C)ccnc21 ; CC1(C)C(=O)N(c2ccc(C3(C#N)CCC3)cc2)C(=O)N1Cc1ccnc2c1CC(=O)N2]

Step 1: tert-butyl 4-((3-(4-(1-cyanocyclobutyl)phenyl)-5,5-dimethyl-2,4-dioxoimidazolidin-1-yl)methyl)-1H-pyrrolo[2,3-b]pyridine-1-carboxylate: Prepared according to General Procedure D using DMF (3.00 mL), 1-(4-(4,4-dimethyl-2,5-dioxoimidazolidin-1-yl)phenyl)cyclobutane-1-carbonitrile (Example A-3, 150 mg, 0.529 mmol), cesium carbonate (704 mg, 2.12 mmol), and tert-butyl 4-(chloromethyl)-1H-pyrrolo[2.3-b]pyridine-1-carboxylate (141 mg, 0.529 mmol) to afford the product (248 mg, 91%). LCMS MS (m/z). [M+H-Boc]⁺=414.3.

Step 2: The crude material from step 1 (248 mg, 0.483 mmol) was dissolved in MeOH (2.41 mL), and hydrochloric acid (1.21 mL, 4.83 mmol, 4 M in dioxanes) was added dropwise. The resulting reaction was stirred overnight at rt. The volatiles were removed under reduced pressure to afford the product (221 mg, >99%). LCMS MS (m/z) [M+H]⁺=414.4.

Step 3: The title product was synthesized according to the procedures of Example 4 (General Procedure Q) using the crude material from the previous step (200 mg, 0.445 mmol), pyridinium tribromide (948 mg, 2.67 mmol), and tBuOH (7.87 mL) followed by AcOH (9.83 mL) and zinc (175 mg, 2.67 mmol). The crude material was purified by reverse phase chromatography (C18, 10-100% MeCN/0.1% of 10 mM ammonium bicarbonate buffer to afford the title product (38.5 mg, 20%). LCMS MS (m/z): [M+H]⁺=430.4. ¹H NMR (400 MHz, DMSO) δ (ppm): 11.00 (s, 1H), 8.01 (d, J=5.5 Hz, 1H), 7.60-7.56 (m, 2H), 7.53-7.48 (m, 2H), 6.97 (d, J=5.5 Hz, 1H), 4.51 (s, 2H), 3.63 (s, 2H), 2.79-2.70 (m, 2H), 2.68-2.58 (m, 2H), 2.33-2.20 (m, 1H), 2.07-1.94 (m, 1H), 1.38 (s, 6H).

### Example 10: Preparation of 1-((2-aminopyridin-4-yl)methyl)-5,5-dimethyl-3-(4-(1-(trifluoromethyl)cyclopropyl)phenyl)imidazolidine-2,4-dione (Compound 83)

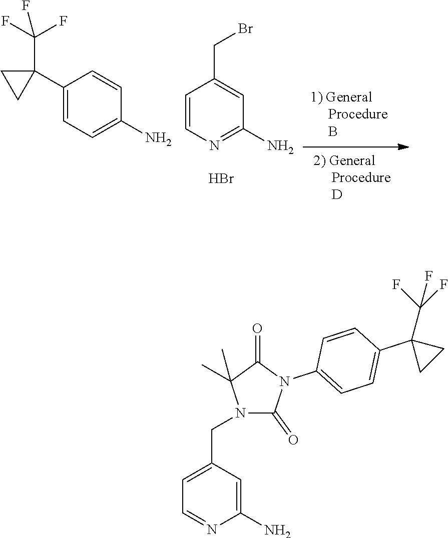

Step 1: Hydantoin core was prepared according to the General Procedure B with 441-(trifluoromethy)cyclopropyl)aniline and methyl α-aminoisobutyrate hydrochloride. The crude material was carried forward as-is.

Step 2: The title compound was prepared according to the General Procedure D with the hydrobromide salt of 4-(bromomethyl)pyridin-2-amine. MS (m/z): [M+H]⁺=419.6, ¹H NMR (400 MHz, DMSO) δ (ppm): 7.84 (d, J=5.3 Hz, 1H), 7.59 (d, J=8.4 Hz, 2H), 7.51-7.42 (m, 2H), 6.49 (dd, J=5.3, 1.4 Hz, 1H), 6.42 (s, 1H), 5.87 (s, 2H), 4.43 (s, 2H), 1.44-1.33 (m, 8H), 1.20-1.14 (m, 2H).

### Example 11: Preparation of 1-((2-(isopropylamino)pyridin-4-yl)methyl)-5,5-dimethyl-3-(4-(1-(trifluoromethyl)cyclopropyl)phenyl)imidazolidine-2,4-dione (Compound 84)

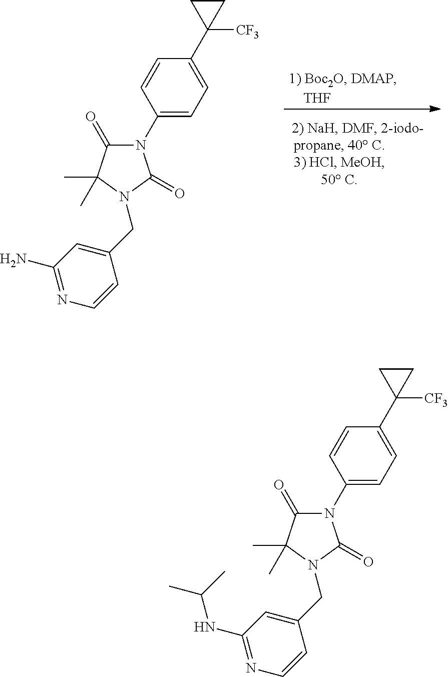 [SMILES: CC1(C)C(=O)N(c2ccc(C3(C(F)(F)F)CC3)cc2)C(=O)N1Cc1ccnc(N)c1 ; CC(C)Nc1cc(CN2C(=O)N(c3ccc(C4(C(F)(F)F)CC4)cc3)C(=O)C2(C)C)ccn1]

Step 1: 1-((2-aminopyridin-4-yl)methyl)-5,5-dimethyl-3-(4-(1-(trifluoromethyl)cyclopropyl)phenyl)imidazolidine-2,4-dione (Example 10, 150 mg, 358 μmol), Boc-anhydride (86.1 mg, 394 μmol), and 4-dimethylaminopyridine (8.85 mg, 71.7 μmol) were stirred in THF (5.00 mL) at rt for 18 h. The reaction was diluted with EtOAc and washed with sat. NH₄Cl and brine. The organics were dried over sodium sulfate, filtered, and concentrated in vacuo. Carried forward as-is MS (m/z): [M+H]⁺=519.

Step 2: tert-butyl (4-((5,5-dimethyl-2,4-dioxo-3-(4-(1-(trifluoromethyl)cyclopropyl)phenyl)imidazolidin-1-yl)methyl)pyridin-2-yl)(isopropyl)carbamate: The crude product from step 1 was taken up in DMF (5 mL) and NaH (35.8 mg, 896 μmol) was added at room temperature. After 20 mins 2-iodopropane (181 μL, 1.79 mmol) was added and the reaction heated to 40° C. for 2 h. The solvents were removed in vacuo and the residue taken up in EtOAc. The organic portion was washed with sat. NH₄Cl, brine, then dried over sodium sulfate, filtered, and purified by flash chromatography (SiO₂. Heptanes/EtOAc 2%-60%) to afford the title compound as a yellow oil (100 mg, 50%). MS (m/z): [M+H]⁺=561.

Step 3: 1-((2-(isopropylamino)pyridin-4-yl)methyl)-5,5-dimethyl-3-(4-(1-(trifluoromethyl)cyclopropyl)phenyl)imidazolidine-2,4-dione: The compound from step 2 (100 mg, 178 μmol) was stirred in MeOH (3.31 mL) at room temperature and HCl (669 μL, 2.68 mmol, 4M dioxane) was added dropwise. The reaction was heated to 50° C. for 2 h. Reaction was cooled to room temperature and diluted with EtOAc, washed with sat. NaHCO₃ and brine. The organics were dried over sodium sulfate, filtered, and concentrated in vacuo. The residue was purified (C18, 45-65% 10 mM ammonium bicarbonate in MeCN) to afford the title compound (32.0 mg, 39%). MS (m/z): [M+H]⁺−461.4, ¹H NMR (400 MHz, DMSO) δ (ppm): 7.87 (d, J=5.2 Hz, 1H), 7.58 (d, J=8.2 Hz, 2H), 7.50-7.39 (m, 2H), 6.42 (d, J=5.2 Hz, 1H), 6.38 (s, 1H), 6.25 (d, J=7.4 Hz, 1H), 4.41 (s, 2H), 4.03-3.86 (m, 1H), 1.43-1.32 (m, 8H), 1.19-1.13 (m, 2H), 1.10 (d, J=6.4 Hz, 6H).

### Example 12: Preparation of 1-((2-(isopropylamino)pyridin-4-yl)methyl)-5,5-dimethyl-3-(4-(trimethylsilyl)phenyl)imidazolidine-2,4-dione (Compound 89)

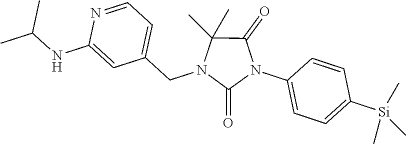 [SMILES: CC(C)Nc1cc(CN2C(=O)N(c3ccc([Si](C)(C)C)cc3)C(=O)C2(C)C)ccn1]

The title compound was prepared from 5,5-dimethyl-3-(4-(trimethylsilyl)phenyl)imidazolidine-2,4-dione and 4-(chloromethyl)-N-isopropylpyridin-2-amine hydrochloride according to General Procedure D. MS (m/z): [M+H]⁺=425.6.

### Example 13: Preparation of 1-((2-(cyclopropylamino)pyridin-4-yl)methyl)-5,5-dimethyl-3-(4-(1-(trifluoromethyl)cyclopropyl)phenyl)imidazolidine-2,4-dione (Compound 93)

### Step 1:

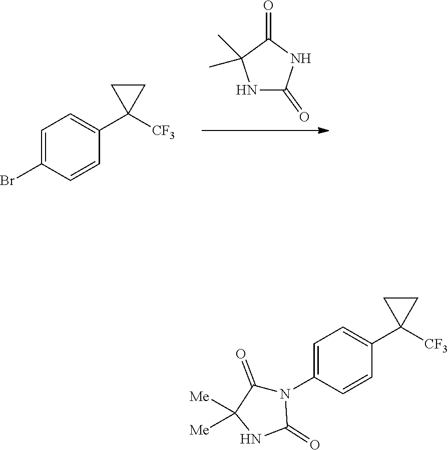

To 1-Bromo-4-(1-(trifluoromethyl)cyclopropyl)benzene in DMF (110 mL) was charged 5,5-dimethylhydantoin and Copper(I) oxide. The reaction was heated to 150° C. for 16 h. When the reaction was complete the reaction mixture was cooled to RT and filtered. The solids were washed with DMF. To the combined filtrates was charged a solution of water and ammonium hydroxide (28%) and the suspension was stirred 90 min at room temperature. The suspension was filtered, and the solids were washed twice with a solution of ammonium hydroxide (28%) in water. The solids were collected and dried to provide 5,5-dimethyl-3-(4-(1-(trifluoromethyl)cyclopropyl)phenyl)imidazolidine-2,4-dione and used in the next step without further purifications. MS (m/z): (M+H)=313.3, ¹H NMR (400 MHz, DMSO-d₆): δ 8.56 (s, 1H), 7.58-7.53 (m, 2H), 7.42-7.37 (m, 2H), 1.40 (s, 6H), 1.36 (q, J=5.1 Hz, 2H), 1.19-1.14 (m, 2H).

### Step 2:

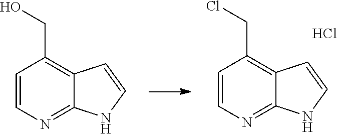 [SMILES: OCc1ccnc2[nH]ccc12 ; ClCc1ccnc2[nH]ccc12 ; Cl]

To a solution of (1H-pyrrolo[2,3-b]pyridin-4-yl)methanol in toluene was charged SOCl₂ and the reaction mixture was heated to 50° C. for 2 h. The reaction mixture was cooled to RT and the resulting solids were filtered and washed with toluene (2×) to provide 4-(chloromethyl)-1H-pyrrolo[2,3-b]pyridine HCl salt and used in the next steps without further purifications. MS (m/z): (M+H)=166.9. ¹H NMR (400 MHz, DMSO-d₆): δ 12.29 (s, 1H), 8.33 (d, J=5.2 Hz, 1H), 7.66-7.60 (m, 1H), 7.31 (d, J=5.2 Hz, 1H), 6.77 (dd, J=3.5, 1.7 Hz, 1H), 5.12 (s, 2H).

### Step 3:

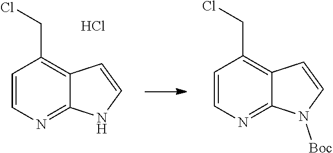 [SMILES: ClCc1ccnc2[nH]ccc12 ; Cl ; CC(C)(C)OC(=O)n1ccc2c(CCl)ccnc21]

To a solution of 4-(chloromethyl)-1H-pyrrolo[2,3-b]pyridine HCl salt and triethylamine in THF was charged 4-Dimethylaminopyridine and Boc-Anhydride and the reaction heated to 35° C. for 2 h. The reaction mixture was cooled and the suspension filtered, and the solids were washed with THF. The combined filtrates were concentrated and dried to provide tert-butyl 4-(chloromethyl)-1H-pyrrolo[2,3-b]pyridine-1-carboxylate and used in the next step without further purifications. MS (m/z): (M+H-tBu)=210.9, 212.9. ¹H NMR (400 MHz, DMSO-d₆): δ 8.40 (d, J=4.9 Hz, 1H), 7.85 (d, J=4.1 Hz, 1H), 7.35 (d, J=4.9 Hz, 1H), 6.89 (d, J=4.1 Hz, 1H), 5.06 (s, 2H), 1.61 (s, 9H).

### Step 4:

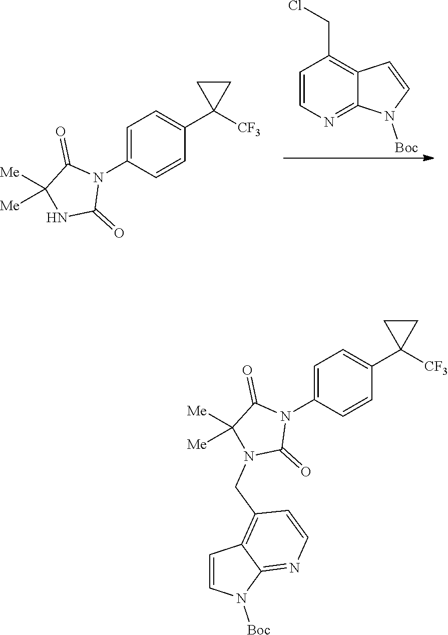 [SMILES: CC1(C)NC(=O)N(c2ccc(C3(C(F)(F)F)CC3)cc2)C1=O ; CC(C)(C)OC(=O)n1ccc2c(CCl)ccnc21 ; CC(C)(C)OC(=O)n1ccc2c(CN3C(=O)N(c4ccc(C5(C(F)(F)F)CC5)cc4)C(=O)C3(C)C)ccnc21]

To 5,5-dimethyl-3-(4-(1-(trifluoromethyl)cyclopropyl)phenyl)imidazolidine-2,4-dione in DMF was charged tert-butyl 4-(chloromethyl)-1H-pyrrolo[2,3-b]pyridine-1-carboxylate and Cesium carbonate and the reaction was heated to 50° C. for 2 h. The reaction mixture was cooled to RT and filtered and washed with DMF. To the filtrate was added saturated aqueous NH₄Cl and the suspension stirred for 30 min. The suspension was filtered and washed with water. The solids were collected and dried to provide tert-butyl 4-((5,5-dimethyl-2,4-dioxo-3-(4-(1-(trifluoromethyl)cyclopropyl)phenyl)imidazolidin-1-yl)methyl)-1H-pyrrolo[2,3-b]pyridine-1-carboxylate is isolated and used in the next step without further purifications. MS (m/z): (M+H)=543.4. ¹H NMR (400 MHz, DMSO-d₆): δ 8.36 (d, J=4.9 Hz, 1H), 7.84 (d, J=4.1 Hz, 1H), 7.63-7.57 (m, 2H), 7.53-7.46 (m, 2H), 7.36 (d, J=5.0 Hz, 1H), 6.93 (d, J=4.1 Hz, 1H), 4.90 (s, 2H), 1.61 (s, 9H), 1.41-1.31 (m, 8H), 1.22-1.14 (m, 2H).

### Step 5:

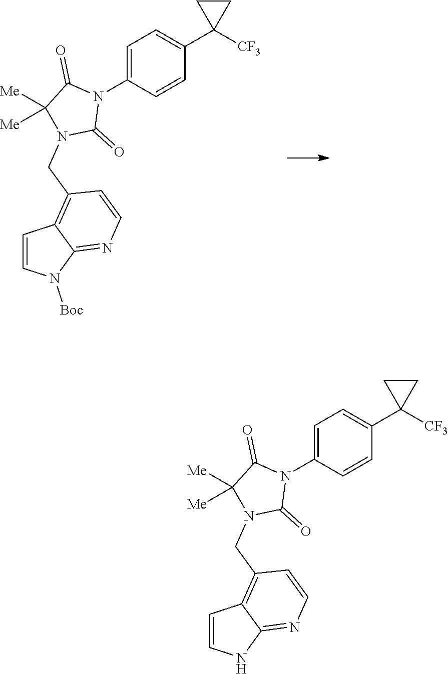 [SMILES: CC(C)(C)OC(=O)n1ccc2c(CN3C(=O)N(c4ccc(C5(C(F)(F)F)CC5)cc4)C(=O)C3(C)C)ccnc21 ; CC1(C)C(=O)N(c2ccc(C3(C(F)(F)F)CC3)cc2)C(=O)N1Cc1ccnc2[nH]ccc12]

To tert-butyl 4-((5,5-dimethyl-2,4-dioxo-3-(4-(1-(trifluoromethyl)cyclopropyl)phenyl)imidazolidin-1-yl)methyl)-1H-pyrrolo[2,3-b]pyridine-1-carboxylate in MeOH was charged HCl (4M in dioxane) and the reaction heated to 50° C. for 2 h. The reaction mixture was cooled and concentrated. The residue is diluted with water and saturated NaHCO₃. The suspension was stirred, filtered, and the solids washed with water (2×) then EtOAc. The solids were collected and dried to provide 1-((1H-pyrrolo[2,3-b]pyridin-4-yl)methyl)-5,5-dimethyl-3-(4-(1-(trifluoromethyl)cyclopropyl)phenyl)imidazolidine-2,4-dione and used in the next step without further purifications. MS (m/z): (M+H)=443.3. ¹H NMR (400 MHz, DMSO-d₆): δ 12.36 (s, 1H), 8.32 (d, J=5.5 Hz, 1H), 7.68-7.63 (m, 1H), 7.63-7.57 (m, 2H), 7.53-7.46 (m, 2H), 7.37 (d, J=5.5 Hz, 1H), 6.85 (dd, J=3.4, 1.6 Hz, 1H), 4.99 (s, 2H), 1.42-1.35 (m, 8H), 1.21-1.15 (m, 2H).

### Step 6:

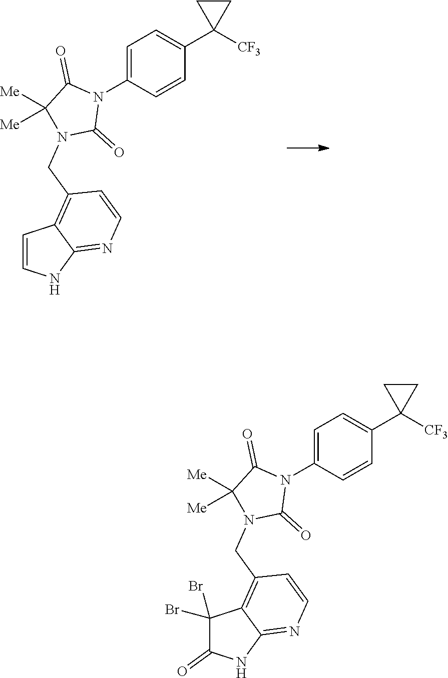 [SMILES: CC1(C)C(=O)N(c2ccc(C3(C(F)(F)F)CC3)cc2)C(=O)N1Cc1ccnc2[nH]ccc12 ; CC1(C)C(=O)N(c2ccc(C3(C(F)(F)F)CC3)cc2)C(=O)N1Cc1ccnc2c1C(Br)(Br)C(=O)N2]

To a solution of 1-((1H-pyrrolo[2,3-b]pyridin-4-yl)methyl)-5,5-dimethyl-3-(4-(1-(trifluoromethyl)cyclopropyl)phenyl)imidazolidine-2,4-dione in THF and H₂O was charged Pyridinium tribromide and the reaction was stirred at RT 2.5 h. The reaction was diluted with water and the resulting precipitate was collected and dried to provide 1-((3,3-dibromo-2-oxo-2,3-dihydro-1H-pyrrolo[2,3-b]pyridin-4-yl)methyl)-5,5-dimethyl-3-(4-(1-(trifluoromethyl)cyclopropyl)phenyl)imidazolidine-2,4-dione and used in the next step without further purifications. MS (m/z): (M−H)=615.1. ¹H NMR (400 MHz, DMSO-d₆): δ 1H NMR (400 MHz, DMSO d₆) δ 12.14 (s, 1H), 8.21 (d, J=5.6 Hz, 1H), 7.61 (app d, J=8.4 Hz, 2H), 7.53 (app d, J=8.4 Hz, 2H), 7.25 (d, J=5.6 Hz, 1H), 4.88 (s, 2H), 1.52-1.43 (m, 6H), 1.37 (app t, J=5.9 Hz, 2H), 1.23-1.13 (m, 2H).

### Step 7:

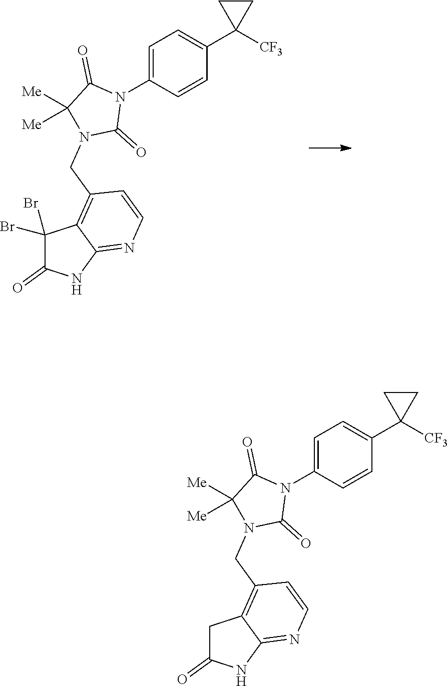 [SMILES: CC1(C)C(=O)N(c2ccc(C3(C(F)(F)F)CC3)cc2)C(=O)N1Cc1ccnc2c1C(Br)(Br)C(=O)N2 ; CC1(C)C(=O)N(c2ccc(C3(C(F)(F)F)CC3)cc2)C(=O)N1Cc1ccnc2c1CC(=O)N2]

To a solution of 1-((3,3-dibromo-2-oxo-2,3-dihydro-1H-pyrrolo[2,3-b]pyridin-4-yl)methyl)-5,5-dimethyl-3-(4-(1-(trifluoromethyl)cyclopropyl)phenyl)imidazolidine-2,4-dione in acetic acid was charged zinc powder and the reaction was stirred at RT 8 h. The reaction mixture was filtered and the solids washed with water then acetone. The solids were collected and dried to provide 5,5-dimethyl-1-((2-oxo-2,3-dihydro-1H-pyrrolo[2,3-b]pyridin-4-yl)methyl)-3-(4-(1-(trifluoromethyl)cyclopropyl)phenyl)imidazolidine-2,4-dione. MS (m/z): (M+H)=459.1. ¹H NMR (400 MHz, DMSO-d₆): δ 11.01 (s, 1H), 8.02 (d, J=5.5 Hz, 1H), 7.59 (d, J=8.4 Hz, 2H), 7.48 (d, J=8.4 Hz, 2H), 6.97 (d, J=5.5 Hz, 1H), 4.53 (s, 2H), 3.64 (s, 2H), 1.91 (s, AcOH), 1.43-1.34 (m, 8H), 1.21-1.15 (m, 2H).

### Example 14: Preparation of (R)-1-((2-((1-methoxypropan-2-yl)amino)pyridin-4-yl)methyl)-5,5-dimethyl-3-(4-(1-(trifluoromethyl)cyclopropyl)phenyl)imidazolidine-2,4-dione (Compound 114)

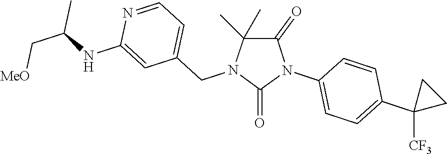 [SMILES: COC[C@@H](C)Nc1cc(CN2C(=O)N(c3ccc(C4(C(F)(F)F)CC4)cc3)C(=O)C2(C)C)ccn1]

The title compound was prepared according to General Procedure F, starting from 5,5-dimethyl-3-(4-(1-(trifluoromethyl)cyclopropyl)phenyl)imidazolidine-2,4-dione and (R)-1-Methoxy-2-propylamine. MS (m/z): [M−H]⁺=491.3.

### Example 15: Preparation of 1-((2-(isopropylamino)-3-methylpyridin-4-yl)methyl)-5,5-dimethyl-3-(4-(1-(trifluoromethyl)cyclopropyl)phenyl)imidazolidine-2,4-dione (Compound 122)

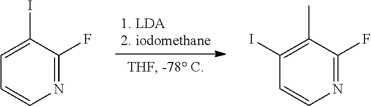

Step 1: 2-fluoro-4-iodo-3-methylpyridine: A solution of diisopropylamine (381 μL, 2.69 mmol) in THF (11.2 mL) was cooled to −78° C. and n-butyllithium (2.5 M in hexanes, 1.08 mL, 2.69 mmol) was added. Then 2-fluoro-3-iodopyridine (499 mg, 2.24 mmol) was added, the reaction was stirred for 5 minutes at room temperature then 1 hour at −78° C. Iodomethane (282 μL, 4.48 mmol) was added and the mixture was warmed to room temperature and stirred for 20 minutes. The reaction was filtered through a plug of silica and eluted with DCM to afford the title compound as a colorless oil (438 mg, 82%) MS (m/z): [M+H]+=238.0.

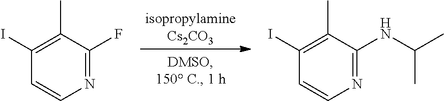

Step 2: 4-iodo-N-isopropyl-3-methylpyridin-2-amine: To a solution of 2-fluoro-4-iodo-3-methylpyridine (438 mg, 1.85 mmol) and isopropylamine (633 μL, 7.39 mmol) in DMSO (7.4 mL) was added cesium carbonate (608 mg, 1.85 mmol). The mixture was sealed and heated to 150° C. under microwave conditions for 1 hour. The reaction was diluted with EtOAc and washed with water (3×), then dried over MgSO₄, filtered, and concentrated. The residue was purified by flash chromatography (SiO₂, 2-15% EtOAc in hexanes) to give the title compound (75.0 mg, 15%) as a colorless oil. MS (m/z): [M+H]⁺=277.0.

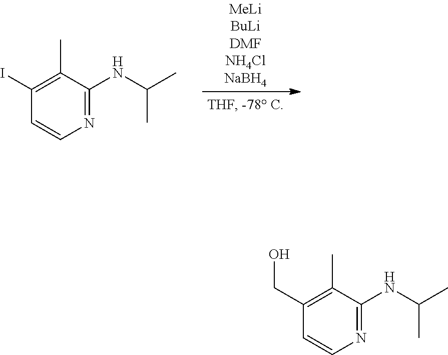

Step 3: (2-(isopropylamino)-3-methylpyridin-4-yl)methanol: A solution of 4-iodo-N-isopropyl-3-methylpyridin-2-amine (75.0 mg, 272 μmol) in THF (3.00 mL) was cooled to −78° C. To the reaction mixture was added sequentially MeLi (3 M in DME, 87.6 μL, 272 μmol), n-butyllithium (217 μL, 543 μmol), then dimethylformamide (211 μL, 2.72 mmol). The reaction was then warmed to room temperature before adding sat. NH₄Cl (100 μL) followed by NaBH₄ (10×) mg, 2.6 mmol). The reaction was stirred for 5 minutes then quenched with 10% citric acid, concentrated and purified by flash chromatography (SiO₂, 0-25% MeOH in DCM containing 2% NH₄OH) to give the title compound (6.00 mg, 12%) as a colorless oil. MS (m/z): [M+H]⁺=181.1.

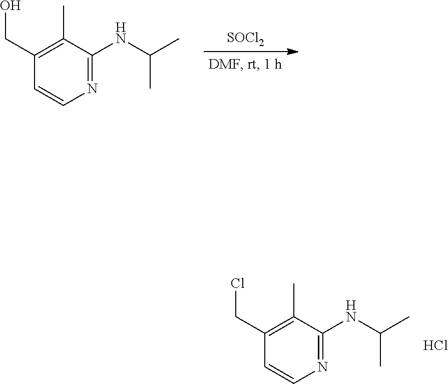

Step 4: 4-(chloromethyl)-N-isopropyl-3-methylpyridin-2-amine hydrochloride: To a solution of (2-(isopropylamino)-3-methylpyridin-4-yl)methanol (6.00 mg, 33.3 μmol) in CH₃CN (1.00 mL) was added thionyl chloride (12.5 μL, 166 μmol). The mixture was stirred for 1 hour then the volatiles were removed in vacuo to afford the title compound which was used without further purification. MS (m/z): [M+H]⁺=199.2.

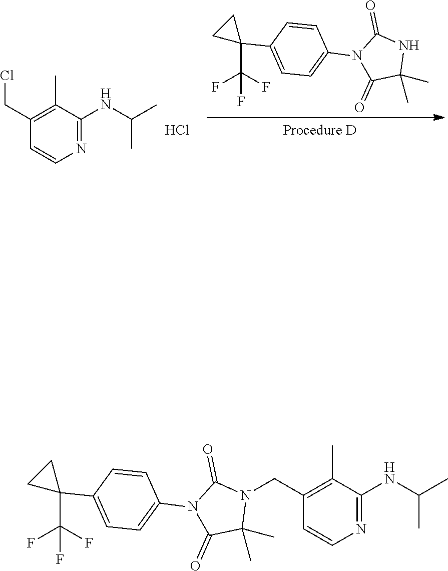 [SMILES: Cc1c(CCl)ccnc1NC(C)C ; Cl ; CC1(C)NC(=O)N(c2ccc(C3(C(F)(F)F)CC3)cc2)C1=O ; Cc1c(CN2C(=O)N(c3ccc(C4(C(F)(F)F)CC4)cc3)C(=O)C2(C)C)ccnc1NC(C)C]

Step 5: 1-((2-(isopropylamino)-3-methylpyridin-4-yl)methyl)-5,5-dimethyl-3-(4-(1-(trifluoromethyl)cyclopropyl)phenyl)imidazolidine-2,4-dione: The title compounds was prepared according to the General Procedure D using the hydrochloride salt of 4-(chloromethyl)-N-isopropyl-3-methylpyridin-2-amine (6 mg, 0.033 mmol) and 5,5-dimethyl-3-(4-(1-(trifluoromethyl)cyclopropyl)phenyl)imidazolidine-2,4-dione (26 mg, 0.083 mmol). The product was purified by flash chromatography (SiO₂, 0-15% MeOH in DCM) to give the title compound (15.0 mg, 91%). MS (m/z): [M+H]⁺=475.5 ¹H NMR (500 MHz, DMSO-d₆) δ (ppm): 7.83 (d, J=5.4 Hz, 1H), 7.59 (d, J=8.4 Hz, 2H), 7.48-7.45 (m, 2H), 6.54 (d, J=3.9 Hz, 1H), 5.53 (broad s, 1H), 4.50 (s, 2H), 4.19 (m, 1H), 2.06 (s, 3H), 1.42-1.34 (m, 8H), 1.23-1.15 (m, 8H).

### Example 16: Preparation of 1-((2-(isopropylamino)pyridin-4-yl)methyl)-5,5-dimethyl-3-(1′-(methylsulfonyl)spiro[cyclobutane-1,3′-indolin]-6′-yl)imidazolidine-2,4-dione (Compound 195)

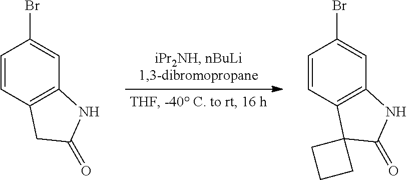 [SMILES: O=C1Cc2ccc(Br)cc2N1 ; O=C1Nc2cc(Br)ccc2C12CCC2]

Step 1: 6′-bromospiro[cyclobutane-1,3′-indolin]-2′-one: The title compound was prepared from 6-bromoindolin-2-one according to General Procedure S. MS (m/z): [M+H]⁺: 252.0.

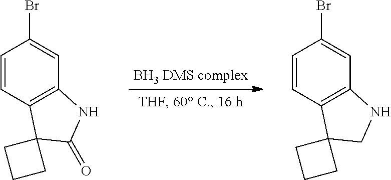 [SMILES: O=C1Nc2cc(Br)ccc2C12CCC2 ; Brc1ccc2c(c1)NCC21CCC1]

Step 2: 6′-bromospiro[cyclobutane-1,3′-indoline]: The title compound was prepared from 6′-bromospiro[cyclobutane-1,3′-indolin]-2′-one according to General Procedure N. MS (m/z): [M+H]₊: 240.1.

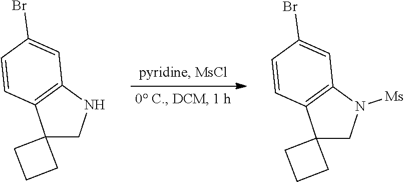 [SMILES: Brc1ccc2c(c1)NCC21CCC1 ; CS(=O)(=O)N1CC2(CCC2)c2ccc(Br)cc21]

Step 3: 6′-bromo-1′-(methylsulfonyl)spiro[cyclobutane-1,3′-indoline]: The title compound was prepared from 6′-bromospiro[cyclobutane-1,3′-indoline] according to General Procedure O. MS (m/z): [M+H]: No ionization. ¹H NMR (400 MHz, CDCl3) δ 7.53 (d, J=1.7 Hz, 1H), 7.28 (d, J=8.0 Hz, 1H), 7.23 (dd, J=8.0, 1.7 Hz, 1H), 4.01 (s, 3H), 2.88 (s, 4H), 2.43-2.20 (m, 6H), 2.12-1.92 (m, 3H).

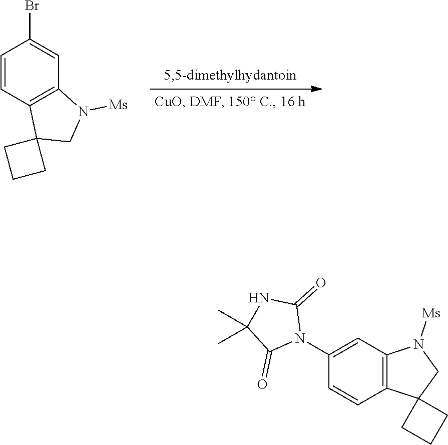 [SMILES: CS(=O)(=O)N1CC2(CCC2)c2ccc(Br)cc21 ; CC1(C)NC(=O)N(c2ccc3c(c2)N(S(C)(=O)=O)CC32CCC2)C1=O]

Step 4: 5.5-dimethyl-3-(1′-(methylsulfonyl[spirolcyclobutane-1,3′-indolin]-6′-yl)imidazolidine-2.4-dione: The title compound was prepared from 6′-bromo-F′-(methylsulfonyl)spiro[cyclobutane-1,3′-indoline] according to General Procedure A. MS (m/z): [M+H]⁺: 364.2.

Step 5: 1-((2-(isopropylamino)pyridin-4-yl)methyl)-5,5-dimethyl-3-(1′-(methylsulfonyl)spiro[cyclobutane-1,3′-indolin]-6′-yl)imidazolidine-2,4-dione

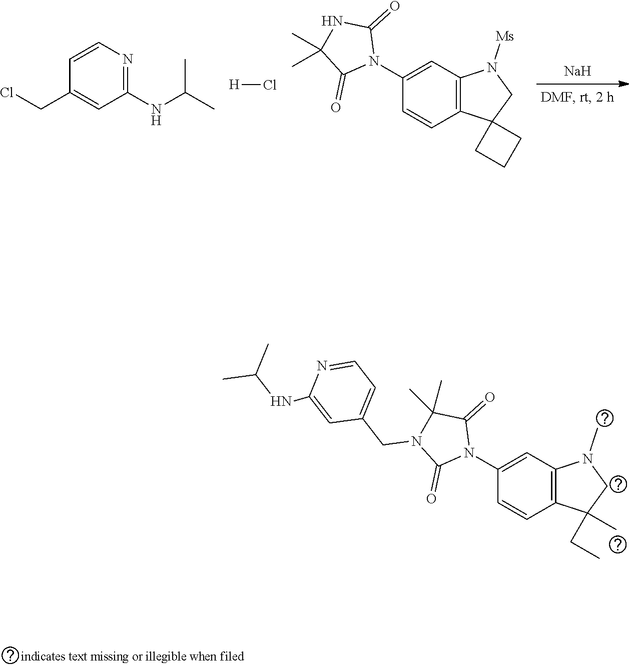

The title compound was prepared from 5,5-dimethyl-3-(1′-(methylsulfonyl)spiro[cyclobutane-1,3′-indolin]-6′-yl)imidazolidine-2,4-dione and 4-(chloromethyl)-N-isopropylpyridin-2-amine hydrochloride according to General Procedure D. MS (m/z): [M+H]⁺: 512.4.

### Example 17: Preparation of 1-((2-((2-methoxy-2-methylpropyl)amino)pyridin-4-yl)methyl)-5,5-dimethyl-3-(1′-(methylsulfonyl)spiro[cyclobutane-1,3′-indolin]-6′-yl)imidazolidine-2,4-dione (Compound 235)

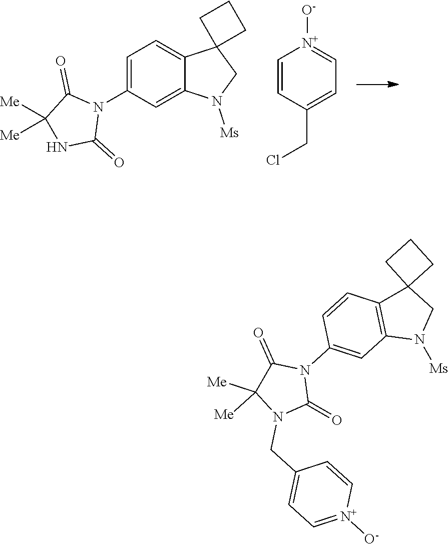

Step-1: 5,5-dimethyl-3-(1′-(methylsulfonyl)spiro[cyclobutane-1,3′-indolin]-6′-yl)imidazolidine-2,4-dione (Example 16, 800 mg, 2.20 mmol), 4-(chloromethyl)pyridine N-oxide (348 mg, 2.42 mmol), and cesium carbonate (1.46 g, 4.40 mmol) in DMF (11.0 mL) was heated to 60° C. for one hour. The reaction mixture was diluted with EtOAc (50 mL), poured into aqueous NH₄Cl, layers separated, and the organic layer was washed with water (25 mL), then brine (25 mL), dried over MgSO₄, filtered, and concentrated under reduced pressure to afford 4-((5,5-dimethyl-3-(1′-(methylsulfonyl)spiro[cyclobutane-1,3′-indolin]-6′-yl)-2,4-dioxoimidazolidin-1-yl)methyl)pyridine 1-oxide (1.00 g, 97%) as a pale tan powder. MS (m/z): [M+H]⁺=471.3.

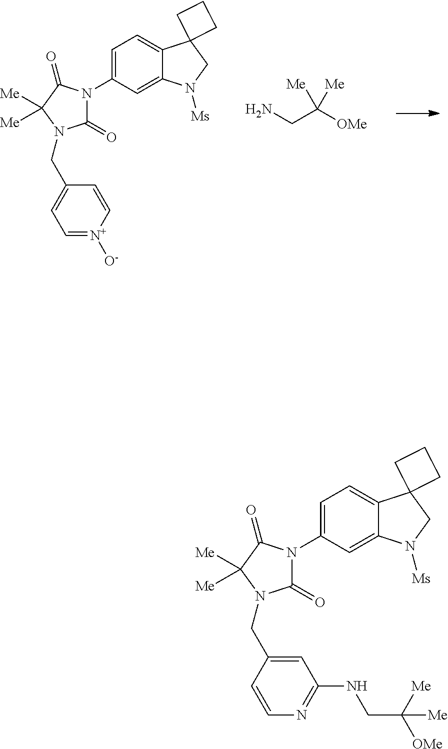 [SMILES: CC1(C)C(=O)N(c2ccc3c(c2)N(S(C)(=O)=O)CC32CCC2)C(=O)N1Cc1cc[n+]([O-])cc1 ; COC(C)(C)CN ; COC(C)(C)CNc1cc(CN2C(=O)N(c3ccc4c(c3)N(S(C)(=O)=O)CC43CCC3)C(=O)C2(C)C)ccn1]

Step-2: Step-1 product (50.0 mg, 106 μmol), 2-methoxy-2-methylpropan-1-amine (57.1 mg, 531 μmol), and N,N-diisopropylethylamine (93.0 μL, 531 μmol) were charged to DCM (2.13 mL) and cooled to 0° C. PyBrop (75.1 mg, 159 μmol) was then added. The reaction was warmed to rt overnight. The reaction mixture was concentrated under reduced pressure and purified by reverse phase chromatography (C18, 5-100% ACN in 10 mM ammonium bicarbonate) to afford the title compound (26.0 mg, 43%) as a white powder. MS (m/z): [M+H]⁺=556.4. ¹H NMR (400 MHz, DMSO) δ (ppm): 7.87 (d, J=5.3 Hz, 1H), 7.66 (d, J=8.1 Hz, 1H), 7.28 (d, J=1.7 Hz, 1H), 7.17 (dd, J=8.0, 1.8 Hz, 1H), 6.60 (s, 1H), 6.44 (dd, J=5.3, 1.2 Hz, 1H), 6.23 (t, J=5.7 Hz, 1H), 4.42 (s, 2H), 4.07 (s, 2H), 3.3-3.31 (overlapped m, 2H), 3.12 (s, 3H), 3.02 (s, 3H), 2.36-2.29 (m, 4H), 2.09-1.97 (m, 2H), 1.40 (s, 6H), 1.11 (s, 6H).

### Example 18: Preparation of 1-((2-(((1S,2R)-2-fluorocyclopropyl)amino)pyridin-4-yl)methyl)-5,5-dimethyl-3-(4-(1-(trifluoromethyl)cyclopropyl)phenyl)imidazolidine-2,4-dione (Compound 142)

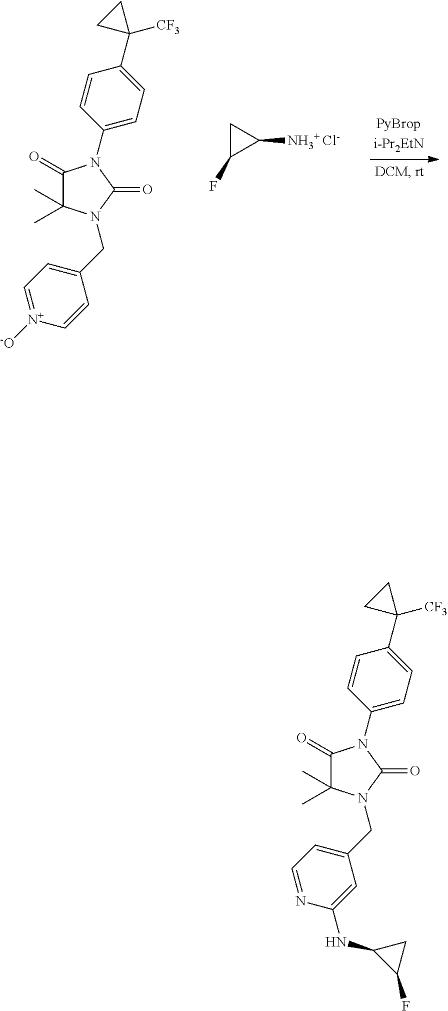

To 1-((2-chloropyridin-4-yl)methyl)-5,5-dimethyl-3-(4-(1-(trifluoromethyl)cyclopropyl)phenyl)imidazolidin-1-yl)methyl)pyridine 1-oxide (100 mg, 238 μmol) (made by procedures analogous to Example 17, Step 1) and (1R,2S)-2-Fluorocyclopropan-1-amine hydrochloride (84 mg, 715 μmol) in DCM (1.19 mL) was added PyBrop (170 mg, 358 μmol) and the mixture was left to stir for 15 minutes. N,N-diisopropylethylamine (209 μL, 1.19 mmol) was added and the mixture was left to stir at room temperature for 4 days. The solvent was removed under vacuum and the crude residue was purified using reverse phase chromatography (C18, 5-100% ACN in 10 mM ammonium bicarbonate) to afford the title compound (4.63 mg, 4%). MS (m/z): [M+H]⁺=477.5 ¹H NMR (400 MHz, DMSO) δ (ppm): 7.95 (d, J=5.7 Hz, 1H), 7.59 (d, J=8.4 Hz, 2H), 7.46 (d, J=8.5 Hz, 2H), 6.80 (d, J=2.9 Hz, 1H), 6.59 (d, J=2.0 Hz, 2H), 4.89-4.61 (m, 1H), 4.48 (s, 2H), 2.68-2.56 (m, 1H), 1.40 (s, 6H), 1.39-1.34 (m, 2H), 1.18 (s, 2H), 1.14-1.02 (m, 1H), 0.95-0.81 (m, 1H).

### Example 19: Preparation of 5,5-dimethyl-1-((2-((1-(pyridin-3-yl)ethyl)amino)pyridin-4-yl)methyl)-3-(4-(1-(trifluoromethyl)cyclopropyl)phenyl)imidazolidine-2,4-dione (Compound 165)

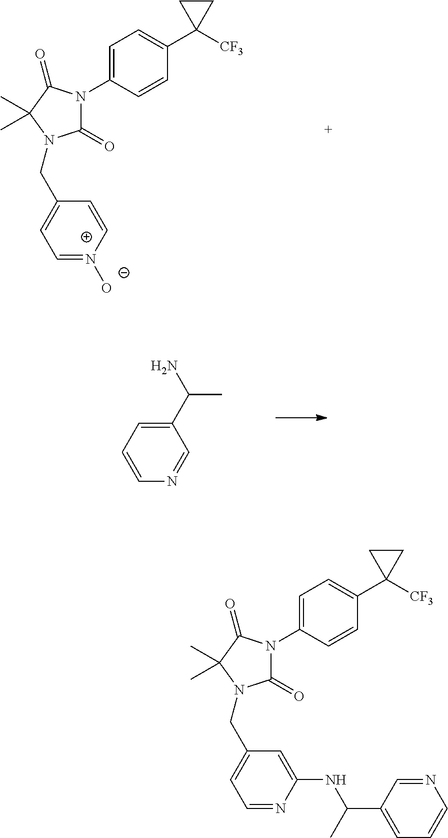

To a solution of 4-((5,5-dimethyl-2,4-dioxo-3-(4-(1-(trifluoromethyl)cyclopropyl)phenyl)imidazolidin-1-yl)methyl)pyridine N-oxide (100 mg, 238 μmol) (made by procedures analogous to Example 17, Step 1) and 1-pyridin-3-yl-ethylamine (89.2 mg, 715 μmol) in DCM (1.20 mL) was added N,N-diisopropylethylamine (209 μL, 1.19 mmol). The reaction mixture was stirred at room temperature for 15 min before the addition of PyBrop (168 mg, 358 μmol). The reaction mixture was stirred at room temperature overnight, concentrated under vacuum, and purified by reverse phase flash chromatography (C₁₈, ammonium bicarbonate buffer/ACN 95/5 to 50/50) to afford the title compound (24 mg, 19%). MS (m/z): [M+H]⁺=524.3. ¹H NMR (400 MHz, DMSO-d₆) δ (ppm): 8.56 (d, J=2.1 Hz, 1H), 8.38 (dd, J=4.7, 1.6 Hz, 1H), 7.84 (d, J=5.2 Hz, 1H), 7.72 (dt, J=7.9, 1.9 Hz, 1H), 7.64-7.56 (m, 2H), 7.50-7.44 (m, 2H), 7.29 (ddd, J=7.9, 4.8, 0.5 Hz, 1H), 7.05 (d, J=7.6 Hz, 1H), 6.49-6.44 (m, 2H), 5.02 (p, J=6.8 Hz, 1H), 4.42 (s, 2H), 1.43 (d, J=7.0 Hz, 3H), 1.38-1.35 (m, 5H), 1.31 (s, 3H), 1.21-1.13 (m, 2H).

### Example 20: Preparation of 1-((2-aminopyridin-4-yl)methyl)-5,5-dimethyl-3-(4-(pentafluoro-16-sulfanyl)phenyl)imidazolidine-2,4-dione (Compound 179)

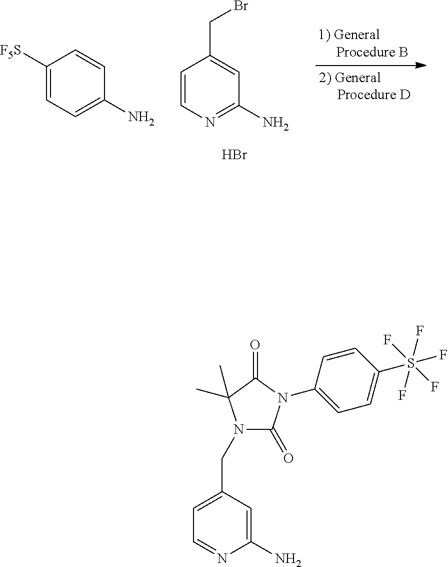

Step 1: Hydantoin core was prepared according to the General Procedure B with 4-aminobenzensulfur pentafluoride and methyl α-aminoisobutyrate hydrochloride. The crude material was carried forward as-is.

Step 2: The title compound was prepared according to the General Procedure D with the hydrobromide salt of 4-(bromomethyl)pyridin-2-amine as alkylating agent. MS (m/z): [M+H]⁺−437.3, ¹H NMR (400 MHz, DMSO) δ (ppm): 8.11-8.02 (m, 2H), 7.82 (d, J=5.2 Hz, 1H), 7.74 (d, J=8.8 Hz, 2H), 6.50 (dd, J=5.3, 1.2 Hz, 1H), 6.42 (s, 1H), 5.85 (s, 2H), 4.43 (s, 2H), 1.39 (s, 6H).

### Example 21: Preparation of 1-((2-(isopropylamino)pyridin-4-yl)methyl)-5,5-dimethyl-3-(4-(pentafluoro-16-sulfanyl)phenyl)imidazolidine-2,4-dione (Compound 178)

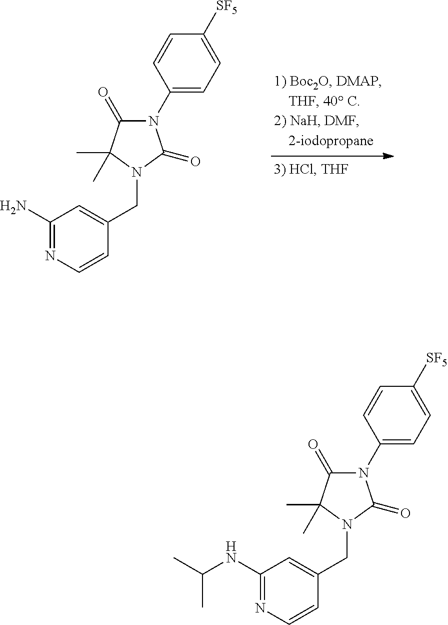

Step 1: Compound 179 (Example 20) (50.0 mg, 115 μmol), Boc-anhydride (30.0 mg, 137 μmol) and 4-Dimethylaminopyridine (4.24 mg, 34.4 μmol) were stirred in THF (3.00 mL) at 40° C. overnight. Reaction was diluted with EtOAc, washed with sat. NH₄Cl and brine. The organics were dried over sodium sulfate, filtered, concentrated in vacuo, and used used without purification. MS (m/z): [M+H]⁺=537.

Step 2: The solid from step 1 was taken up in DMF (3.00 mL) and NaH (9.17 mg, 229 μmol) was added at room temperature. The reaction was stirred for 15 mins and then 2-iodopropane (1.00 mL, 9.90 mmol) was added. Stirring continued for 3 h then the reaction was quenched with MeOH. The volatiles were removed in vacuo and the residue taken up in EtOAc, washed with sat. NH₄Cl and brine. The organics were dried over sodium sulfate, filtered, concentrated in vacuo, and carried forward as-is. MS (m/z): [M+H]⁺=579.

Step 3: The residue from step 2 was taken up in THF (3.00 mL) and HCl (1.00 mL, 4.00 mmol, 4M in dioxane) was added. The reaction was stirred overnight at room temperature and then poured over saturated sodium bicarbonate. The aqueous portion was extracted with EtOAc. The combined organics were washed with brine, dried over sodium sulfate, filtered, and purified by reverse phase chromatography (C18, 10 mM Ammonium Bicarbonate pH 10.3/MeCN, 40%-60%) to afford the title compound (19.2 mg, 35%). MS (m/z): [M+H]⁺=479.2. ¹H NMR (400 MHz, DMSO) δ (ppm): 8.14-8.04 (m, 2H), 7.89 (d, J=5.3 Hz, 1H), 7.76 (d, J=8.9 Hz, 2H), 6.46 (dd, J=5.3, 1.2 Hz, 1H), 6.41 (s, 1H), 6.25 (d, J=7.6 Hz, 1H), 4.44 (s, 2H), 4.05-3.87 (m, 1H), 1.42 (s, 6H), 1.12 (d, J=6.4 Hz, 6H).

### Example 22: Preparation of 1-((2-(((1R,5S,6r)-3-oxabicyclo[3.1.0]hexan-6-yl)amino)pyridin-4-yl)methyl)-3-(3,3-dimethyl-2,3-dihydrobenzofuran-6-yl)-5,5-dimethylimidazolidine-2,4-dione (Compound 229)

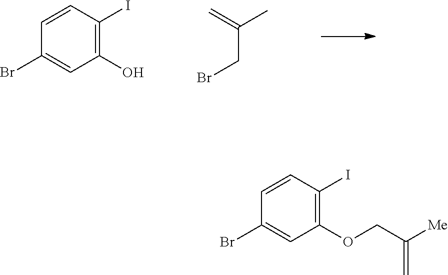

Step-1: To 5-bromo-2-iodophenol (3.00 g, 9.84 mmol), in acetone (32.8 mL) was added potassium carbonate (3.47 g, 24.6 mmol) and potassium iodide (1.83 g, 10.8 mmol). The reaction mixture was stirred for 20 minutes, then 3-bromo-2-methylpropene (1.53 mL, 14.8 mmol) was added and the reaction heated to 65° C. for 3 h. The precipitate removed and washed with acetone (˜25 mL). The combined organics were concentrated under reduced pressure. The resulting residue was dissolved in EtOAc (100 mL), washed with water (2×25 mL), organics dried over MgSO₄, filtered, and concentrated under reduced pressure to afford 4-bromo-1-iodo-2-((2-methylallyl)oxy)benzene (3.45 g, 98%) as a tan powder. MS (m/z): no-ionization. ¹H NMR (400 MHz, CDCl₃) δ (ppm): 7.61 (d, J=8.3 Hz, 1H), 6.91 (d, J=2.1 Hz, 1H), 6.86 (dd, J=8.3, 2.1 Hz, 1H), 5.21-5.18 (m, 1H), 5.05-5.03 (m, 1H), 4.46 (s, 2H), 1.88-1.86 (m, 3H).

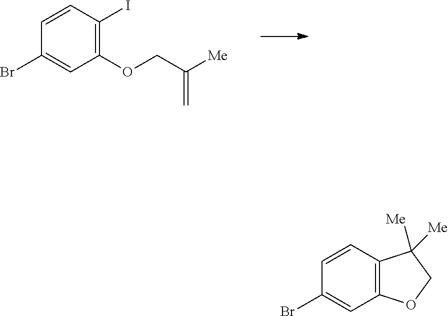

Step-2: To a degassed solution of step-1 product (3.45 g, 9.77 mmol), tetraethylammonium iodide (2.82 g, 10.8 mmol), sodium formate (739 mg, 10.8 mmol), potassium acetate (2.42 g, 24.4 mmol) in DMF (97.7 mL) was added Pd(OAc)₂ (110 mg, 489 μmol). The reaction mixture was heated to 75° C. for 18 h. The reaction mixture was diluted with EtOAc (100 mL), washed with water (2×25 mL). Aqueous layer extracted once more with EtOAc (25 mL), combined organics dried over MgSO₄, filtered, and concentrated under reduced pressure. The crude material was purified by flash chromatography (SiO₂, 0-100% ethyl acetate in hexanes) to afford 6-bromo-3,3-dimethyl-2,3-dihydrobenzofuran (300 mg, 12%) as a pale tan oil. MS (m/z): no ionization. ¹H NMR (400 MHz, CDCl₃) δ (ppm): 7.00 (dd, J=7.9, 1.7 Hz, 1H), 6.96-6.93 (m, 2H), 4.24 (s, 2H), 1.32 (s, 6H).

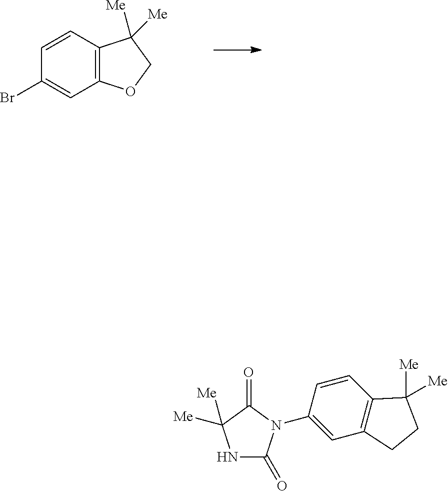 [SMILES: CC1(C)COc2cc(Br)ccc21 ; CC1(C)NC(=O)N(c2ccc3c(c2)CCC3(C)C)C1=O]

Step-3: To step-2 product (300 mg, 1.32 mmol) in DMF (6.61 mL) was added 5,5-dimethylhydantoin (339 mg, 2.64 mmol) and copper(I) oxide (189 mg, 1.32 mmol). The reaction mixture was degassed and heated to 155° C. for 18 h. The reaction mixture was filtered, washed with methanol, volatiles removed under vacuum, and the resulting residue was purified by reverse phase chromatography (C18, 5-100% ACN in 10 mM ammonium bicarbonate) to afford 3-(3,3-dimethyl-2,3-dihydrobenzofuran-6-yl)-5,5-dimethylimidazolidine-2,4-dione (90.0 mg, 25%) as an off-white powder. MS (m/z): [M−H]—=273.0. ¹H NMR (400 MHz, CDCl₃) δ (ppm): 7.15 (d, J=7.9 Hz, 1H), 6.89 (dd, J=7.9, 1.8 Hz, 1H), 6.80 (d, J=1.8 Hz, 1H), 5.48 (s, 1H), 4.27 (s, 2H), 1.54 (s, 6H), 1.34 (s, 6H).

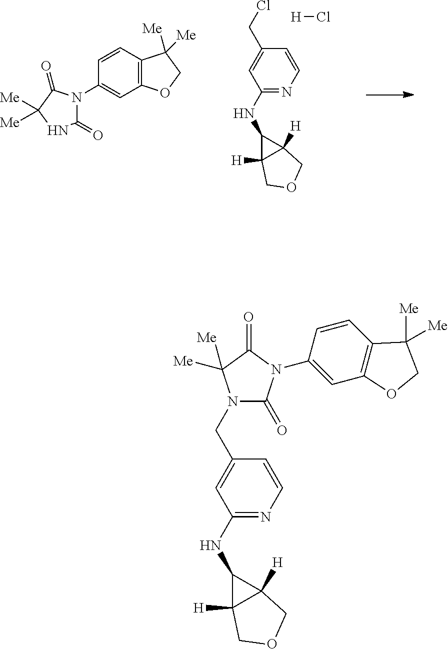

Step-4 To step-3 product (50.0 mg, 182 μmol) in DMF (1.82 mL) was added cesium carbonate (121 mg, 365 μmol), N-((1R,5S,6r)-3-oxabicyclo[3.1.0]hexan-6-yl)-4-(chloromethyl)pyridin-2-amine hydrochloride (47.6 mg, 182 μmol), and the reaction mixture was heated to 55° C. for 1 hour. The reaction mixture was directly purified by reverse phase chromatography (C18, 5-100% ACN in 10 mM ammonium bicarbonate) to afford an off-white material further purified by Prep-HPLC (CSH column, ammonium bicarbonate method) to afford the title compound (36.0 mg, 42%) as a white powder. MS (m/z): [M+H]⁺=463.3. ¹H NMR (400 MHz, DMSO) δ (ppm): 7.94 (d, J=5.1 Hz, 1H), 7.30 (d, J=7.9 Hz, 1H), 6.88 (dd, J=7.9, 1.8 Hz, 1H), 6.81 (d, J=1.7 Hz, 1H), 6.75 (d, J=2.2 Hz, 1H), 6.57 (dd, J=5.2, 1.3 Hz, 1H), 6.50 (s, 1H), 4.48 (s, 2H), 4.28 (s, 2H), 3.86 (d, J=8.4 Hz, 2H), 3.63 (d, J=8.2 Hz, 2H), 2.26-2.23 (m, 1H), 1.75 (s, 2H), 1.37 (s, 6H), 1.32 (s, 6H).

### Example 23: Preparation of 3-(3,3-dimethyl-2,3-dihydrobenzofuran-6-yl)-1-((2-(isopropylamino)pyridin-4-yl)methyl)-5,5-dimethylimidazolidine-2,4-dione (Compound 189)

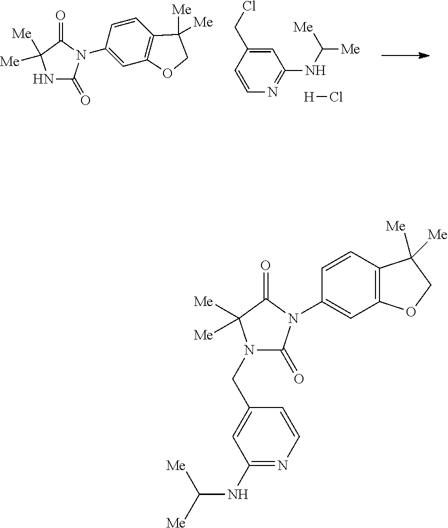

To 3-(3,3-dimethyl-2,3-dihydrobenzofuran-6-yl)-5,5-dimethylimidazolidine-2,4-dione (Example 22, 24.0 mg, 77.0 μmol) in DMF (577 μL) was added NaH (9.24 mg, 231 μmol, 60% dispersion). The slurry was stirred at room temperature for 15 min. Then, a solution of 4-(chloromethyl)-N-isopropylpyridin-2-anine hydrochloride (17.0 mg, 77.0 μmol) in DMF (192 μL) was added dropwise and stirred 1 hour. The reaction mixture was carefully quenched with a few drops of MeOH, and filtered. The volatiles were removed under reduced pressure, and the residue was purified by reverse phase chromatography (C18, 30-50% ACN in 10 mM ammonium bicarbonate) to afford the title compound (8.00 mg, 24%) as a white powder. MS (m/z): [M+H]⁺=423.4. ¹H NMR (400 MHz, DMSO) δ (ppm): 7.88 (d, J=5.2 Hz, 1H), 7.30 (d, J=7.9 Hz, 1H), 6.89 (dd, J=7.9, 1.8 Hz, 1H), 6.82 (d, J=1.7 Hz, 1H), 6.42 (dd, J=5.3, 1.4 Hz, 1H), 6.38 (s, 1H), 6.29 (d, J=7.5 Hz, 1H), 4.40 (s, 2H), 4.28 (s, 2H), 4.01-3.92 (m, 1H), 1.37 (s, 6H), 1.32 (s, 6H), 1.12 (d, J=6.4 Hz, 6H).

### Example 24: Preparation of (S)-3-(3,3-dimethyl-1-(methylsulfonyl)indolin-6-yl)-5,5-dimethyl-1-((2-((tetrahydrofuran-3-yl)amino)pyridin-4-yl)methyl)imidazolidine-2,4-dione (Compound 201)

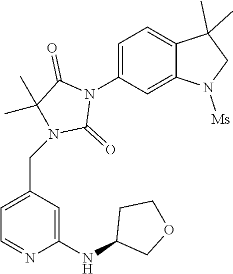 [SMILES: CC1(C)CN(S(C)(=O)=O)c2cc(N3C(=O)N(Cc4ccnc(N[C@H]5CCOC5)c4)C(C)(C)C3=O)ccc21]

The title compound was prepared using the General Procedure J with 3-(3,3-dimethyl-1-(methylsulfonyl)indolin-6-yl)-5,5-dimethylimidazolidine-2,4-dione and (S)-4-(chloromethyl)-N-(tetrahydrofuran-3-yl)pyridin-2-amine. MS (m/z): [M+H]⁺=528.5.

### Example 25: Preparation of (S)-3-((4-((5,5-dimethyl-3-(1′-(methylsulfonyl)spiro[cyclobutane-1,3′-indolin]-6′-yl)-2,4-dioxoimidazolidin-1-yl)methyl)pyridin-2-yl)amino)butanenitrile (Compound 221)

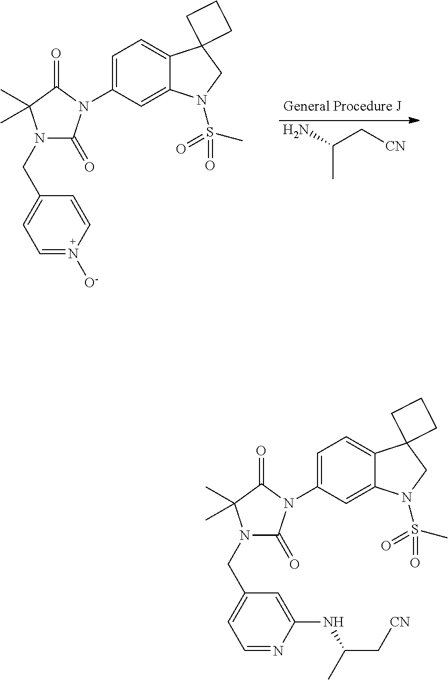

The title product was synthesized according to General Procedure J using 4-((5,5-dimethyl-3-(1′-(methylsulfonyl)spiro[cyclobutane-1,3′-indolin]-6′-yl)-2,4-dioxoimidazolidin-1-yl)methyl)pyridine 1-oxide (129 mg, 0.274 mmol) (made by procedures analogous to Example 17, Step 1), (S)-3-aminobutanenitrile hydrochloride (75.0 mg, 0.603 mmol), DCM (1.50 mL), N,N-diisopropylethylamine (240 μL, 1.37 mmol) and PyBrop (194 mg, 0.411 mmol). The crude material was purified by reverse phase chromatography (C18, 10-100% MeCN/10 mM ammonium bicarbonate) to afford the title product (16.3 mg, 11%). MS (m/z): [M+H]⁺=537.4. ¹H NMR (400 MHz, DMSO) δ (ppm): 7.92 (d, J=5.3 Hz, 1H), 7.64 (d, J=8.1 Hz, 1H), 7.26 (d, J=1.8 Hz, 1H), 7.15 (dd, J=8.0, 1.8 Hz, 1H), 6.72 (s, 1H), 6.57-6.44 (m, 2H), 4.44 (s, 2H), 4.19-4.09 (m, 1H), 4.05 (s, 2H), 3.02-2.97 (m, 3H), 2.89-2.78 (m, 1H), 2.76-2.66 (m, 1H), 2.35-2.26 (m, 4H), 2.05-1.96 (m, 2H), 1.41-1.34 (m, 6H), 1.28-1.18 (m, 3H).

### Example 26: Preparation of 1-((2-((1R,5S,6r)-3-oxabicyclo[3.1.0]hexan-6-ylamino)pyridin-4-yl)methyl)-3-(1′-(cyclopropanecarbonyl)spiro[cyclopropane-1,3′-indolin]-6′-yl)-5,5-dimethylimidazolidine-2,4-dione (Example 231)

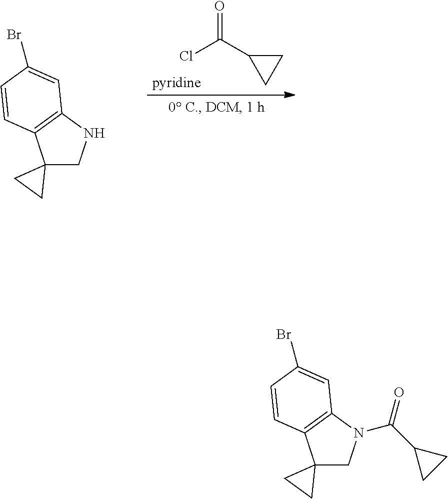 [SMILES: Brc1ccc2c(c1)NCC21CC1 ; O=C(Cl)C1CC1 ; O=C(C1CC1)N1CC2(CC2)c2ccc(Br)cc21]

Step 1: 6′-bromo-1′-(cyclopropanecarbonyl)spiro[cyclopropane-1,3′-indolin]-2′-one: The title compound was prepared from 6′-bromospiro[cyclopropane-1,3′-indoline]according to General Procedure O. MS (m/z): [M+H]⁺=292.1.

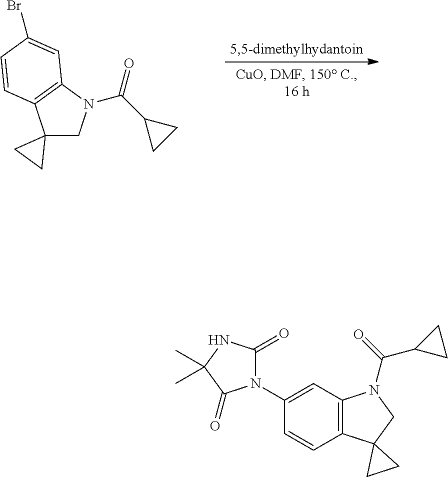 [SMILES: O=C(C1CC1)N1CC2(CC2)c2ccc(Br)cc21 ; CC1(C)NC(=O)N(c2ccc3c(c2)N(C(=O)C2CC2)CC32CC2)C1=O]

Step 2: 3-(1′-(cyclopropanecarbonyl)spiro[cyclopropane-1,3′-indolin]-6′-yl)-5.5-dimethylimidazolidine-2,4-dione: The title compound was prepared from 6′-bromo-1′-(cyclopropanecarbonyl)spiro[cyclopropane-1,3′-indolin]-2′-one according to General Procedure A. MS m/z): [M+H]⁺=340.3.

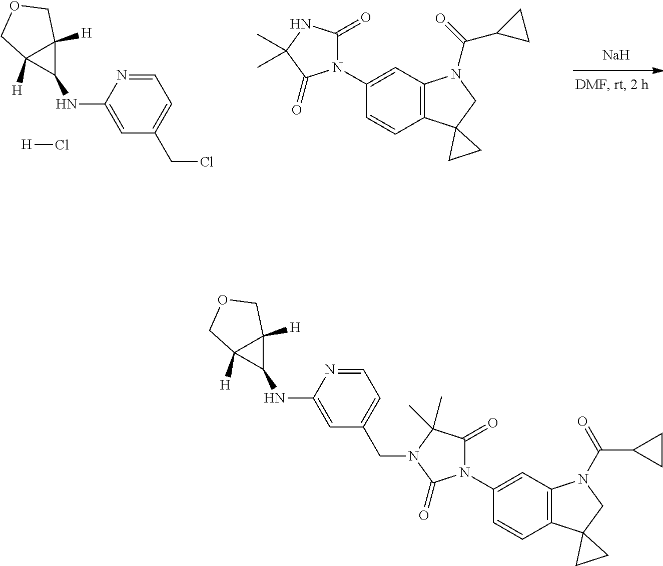

Step 3: 1-((2-((1R,5S,6r)-3-oxabicyclo[3.1.0]hexan-6-ylamino)pyridin-4-yl)methyl)-3-(1′-(cyclopropanecarbonyl)spiro[cyclopropane-1,3′-indolin]-6′-yl)-5,5-dimethylimidazolidine-2,4-dione: The title compound was prepared from 3-(1′-(cyclopropanecarbonyl)spiro[cyclopropane-1,3′-indolin]-6′-yl)-5,5-dimethylimidazolidine-2,4-dione and N-((1R,5S,6r)-3-oxabicyclo[3.1.0]hexan-6-yl)-4-(chloromethyl)pyridin-2-amine hydrochloride according to General Procedure D. MS (m/z): [M+H]⁺=528.4.

### Example 27: Preparation of (R)-3-(3-(tert-butyl)-1-methyl-1H-pyrazol-5-yl)-1-((2-((1-methoxypropan-2-yl)amino)pyridin-4-yl)methyl)-5,5-dimethylimidazolidine-2,4-dione (Compound 259)

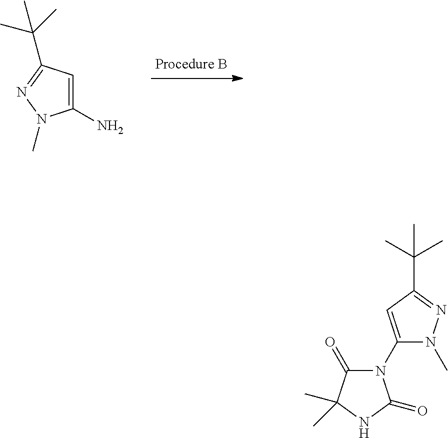

Step 1: 3-(3-(tert-butyl)-1-methyl-1H-pyrazol-5-yl)-5,5-dimethylimidazolidine-2.4-dione: Hydantoin core was prepared according to the General Procedure B with 5-Amino-3-tert-butyl-1-methylpyrazole and methyl α-aminoisobutyrate hydrochloride. The crude material was purified by chromatography (SiO₂, with DCM/20% MeOH in DCM, 2%-50%). MS (m/z): [M+H]⁺=265.

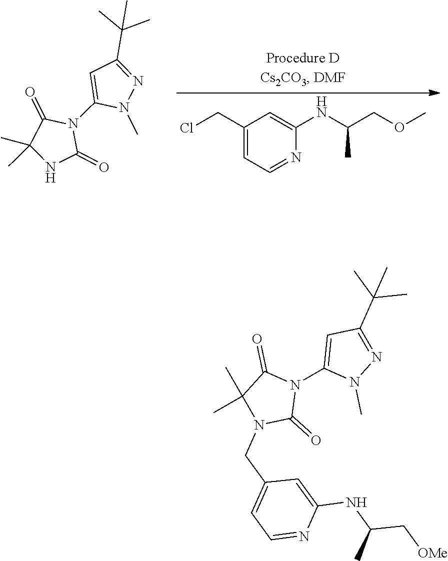 [SMILES: Cn1nc(C(C)(C)C)cc1N1C(=O)NC(C)(C)C1=O ; COC[C@@H](C)Nc1cc(CCl)ccn1 ; COC[C@@H](C)Nc1cc(CN2C(=O)N(c3cc(C(C)(C)C)nn3C)C(=O)C2(C)C)ccn1]

Step 2: The title compound was prepared according to the General Procedure D using 3-(3-(tert-butyl)-1-methyl-1H-pyrazol-5-yl)-5,5-dimethylimidazolidine-2,4-dione, (R)-4-(chloromethyl)-N-(1-methoxypropan-2-yl)pyridin-2-amine as alkylating agent and cesium carbonate as base. MS (m/z): [M+H]⁺=443.3. ¹H NMR (400 MHz, DMSO) δ (ppm): 7.88 (d, J=6.0 Hz, 1H), 6.47-6.39 (m, 2H), 6.31 (d, J=7.9 Hz, 1H), 6.24 (s, 1H), 4.41 (s, 2H), 4.15-4.00 (m, 1H), 3.58 (s, 3H), 3.36 (dd, J=9.2, 5.1 Hz, 1H), 3.23 (s, 3H), 3.19 (dd, J=9.2, 6.2 Hz, 1H), 1.41 (s, 3H), 1.40 (s, 3H), 1.23 (s, 9H), 1.09 (d, J=6.6 Hz, 3H).

### Example 28: Preparation of 3-(4-(tert-butyl)phenyl)-1-((2-(isopropylamino)pyridin-4-yl)methyl)-5,5-dimethylimidazolidine-2,4-dione (Compound 263)

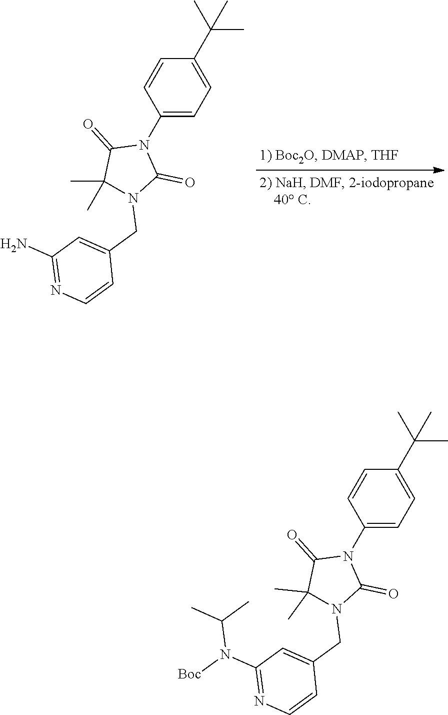 [SMILES: CC(C)(C)c1ccc(N2C(=O)N(Cc3ccnc(N)c3)C(C)(C)C2=O)cc1 ; CC(C)N(C(=O)OC(C)(C)C)c1cc(CN2C(=O)N(c3ccc(C(C)(C)C)cc3)C(=O)C2(C)C)ccn1]

Step 1: 1-((2-aminopyridin-4-yl)methyl)-3-(4-(tert-butyl)phenyl)-5,5-dimethylimidazolidine-2,4-dione, Boc-anhydride (78.6 mg, 360 μmol), and 4-Dimethylaminopyridine (8.08 mg, 65.5 μmol) were stirred in THF (5.00 mL) at room temperature for 18 h. The reaction was diluted with EtOAc and washed with sat. NH₄Cl and brine. Organic portion was separated and dried over sodium sulfate, filtered, and concentrated in vacuo. Carried forward as-is MS (m/z): [M+H]⁺=467.

Step 2: tert-butyl (4-((3-(4-(tert-butyl)phenyl)-5,5-dimethyl-2,4-dioxoimidazolidin-1-yl)methyl)pyridin-2-yl)(isopropyl)carbamate: The product from step 1 was taken up in DMF (5 mL) and NaH (32.7 mg, 819 μmol) was added at room temperature with stirring. After 20 minutes 2-iodopropane (165 μL, 1.64 mmol) was added and the reaction heated to 40° C. for 2 h. The solvents were removed in vacuo and the residue taken up in EtOAc. The organic portion was washed with sat. NH₄Cl and brine, dried over sodium sulfate, filtered, and purified by chromatography (SiO₂, Heptanes/EtOAc: 2%-100%) to afford the title compound (60 mg, 36%). MS (m/z): [M+H]⁺=509.

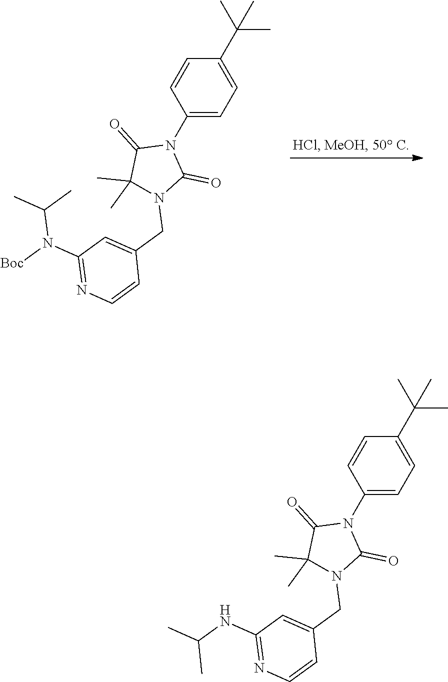 [SMILES: CC(C)N(C(=O)OC(C)(C)C)c1cc(CN2C(=O)N(c3ccc(C(C)(C)C)cc3)C(=O)C2(C)C)ccn1 ; CC(C)Nc1cc(CN2C(=O)N(c3ccc(C(C)(C)C)cc3)C(=O)C2(C)C)ccn1]

Step 3: 3-(4-(tert-butyl)phenyl)-1-((2-(isopropylamino)pyridin-4-yl)methyl)-5.5-dimethylimidazolidine-2,4-dione: tert-butyl (4-((3-(4-(tert-butyl)phenyl)-5,5-dimethyl-2,4-dioxoimidazolidin-1-yl)methyl)pyridin-2-yl)(isopropyl)carbamate (60.0 mg, 118 μmol) was stirred in MeOH (2.19 mL) at room temperature and HCl (442 μL, 1.77 mmol, 4M in dioxane) was added dropwise. The reaction was heated to 50° C. for 3 h. Reaction was cooled to room temperature and diluted with EtOAc, washed with sat. NaHCO₃ and brine. The organic portion was separated and dried over sodium sulfate, filtered, and concentrated in vacuo and purified by reverse phase chromatography (C18, 45-65% 10 mM ammonium bicarbonate in MeCN) to afford the title compound (21.9 mg, 45%). MS(m/z): [M+H]⁺=409.5, ¹H NMR (400 MHz, DMSO) δ (ppm): 7.87 (d, J=5.3 Hz, 1H), 7.54-7.45 (m, 2H), 7.38-7.27 (m, 2H), 6.41 (dd, J=5.3, 1.3 Hz, 1H), 6.38 (s, 1H), 6.27 (d, J=7.6 Hz, 1H), 4.40 (s, 2H), 4.01-3.88 (m, 1H), 1.37 (s, 6H), 1.29 (s, 9H), 1.10 (d, J=6.4 Hz, 6H)

### Example 29: Preparation of (S)-3-((4-((3-(4-(tert-butyl)phenyl)-5,5-dimethyl-2,4-dioxoimidazolidin-1-yl)methyl)pyridin-2-yl)amino)butanenitrile (Compound 278)

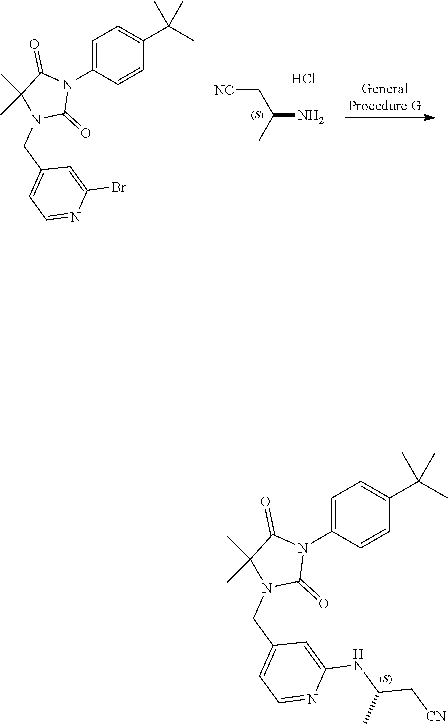

The title compound was prepared according to General Procedure G using (S)-3-aminobutanenitrile hydrochloride (2.2 equiv.) MS (m/z): [M+H]⁺=434.3, ¹H NMR (400 MHz, DMSO) δ (ppm): 7.92 (d, J=5.2 Hz, 1H), 7.62-7.41 (m, 2H), 7.39-7.19 (m, 2H), 6.71 (d, J=6.9 Hz, 1H), 6.57-6.40 (m, 2H), 4.43 (s, 2H), 4.16-4.10 (m, 1H), 2.86-2.68 (m, 2H), 1.37 (d, J=2.9 Hz, 6H), 1.29 (s, 9H), 1.24 (d, J=6.7 Hz, 3H).

### Example 30: Preparation of ((2-aminopyridin-4-yl)methyl)-3-(4-((trifluoromethyl)thio)phenyl)-1,3-diazaspiro[4.4]nonane-2,4-dione (Compound 291)

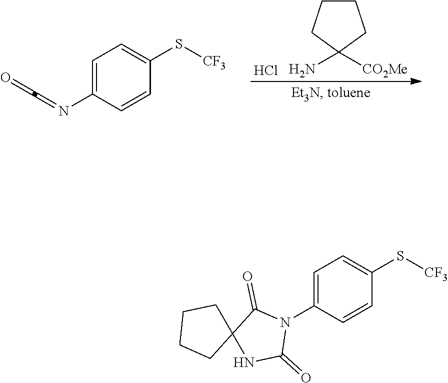

Step 1: 3-(4-((trifluoromethyl)thio)phenyl)-1,3-diazaspiro[4.4]nonane-2,4-dione: The title compound was prepared according to the General Procedure C using cycloleucine HCl salt (1.2 equiv.). MS (m/z): [M−H]⁻=329.1.

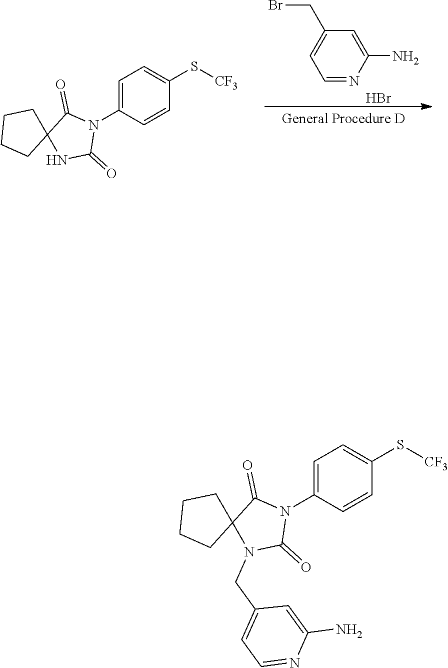 [SMILES: O=C1NC2(CCCC2)C(=O)N1c1ccc(SC(F)(F)F)cc1 ; Nc1cc(CBr)ccn1 ; Br ; Nc1cc(CN2C(=O)N(c3ccc(SC(F)(F)F)cc3)C(=O)C23CCCC3)ccn1]

Step 2: The title compound was prepared according to the General Procedure D using 4-(bromomethyl)pyridin-2-amine hydrobromide (1 equiv.). MS (m/z): [M+H]⁺=437.2. H NMR (400 MHz, DMSO) δ (ppm): 7.85-7.81 (m, 31H), 7.65 (d, J=8.6 Hz, 2H), 6.45 (dd, J=5.3, 1.1 Hz, 1H), 6.37 (s, 1H), 5.89 (s, 21), 4.42 (s, 2H), 2.05-1.99 (m, 2H), 1.95-1.81 (m, 2H), 1.75-1.70 (m, 4H).

### Example 31. Preparation of 1-((2-aminopyridin-4-yl)methyl)-5-ethyl-5-methyl-3-(4-((trifluoromethyl)thio)phenyl)imidazolidine-2,4-dione (Compound 297)

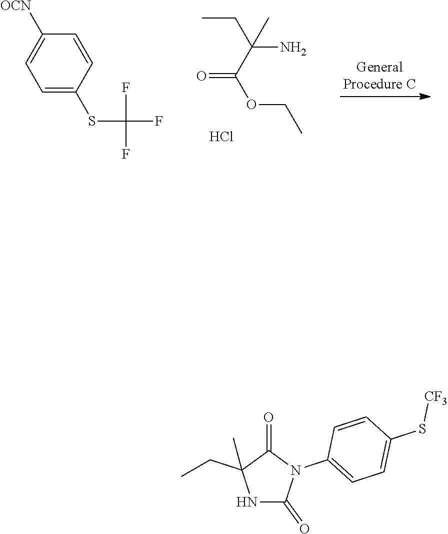

Step 1: Hydantoin core was prepared according to the General Procedure C with 4-(trifluoromethythio)phenyl isocyanate (400 mg) and ethyl 2-amino-2-methylbutanoate hydrochloride (1 equiv.). This reaction required stirring at 80° C. in for 4 h. The crude material was used as-is. MS (m/z): [M+H]⁺=317.

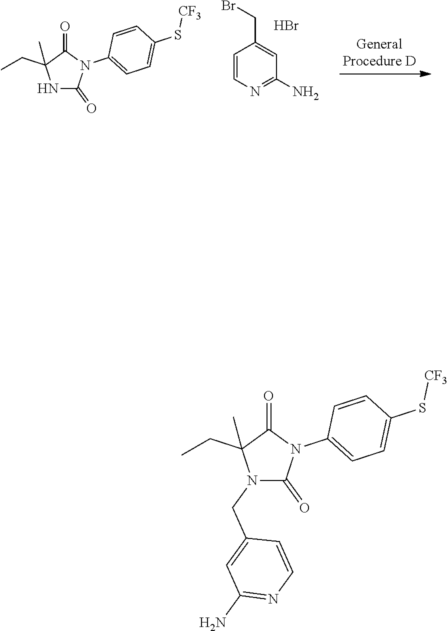 [SMILES: CCC1(C)NC(=O)N(c2ccc(SC(F)(F)F)cc2)C1=O ; Nc1cc(CBr)ccn1 ; Br ; CCC1(C)C(=O)N(c2ccc(SC(F)(F)F)cc2)C(=O)N1Cc1ccnc(N)c1]

Step 2: The title compound was prepared according to the General Procedure D with 5-ethyl-5-methyl-3-(4-((trifluoromethyl)thio)phenyl)imidazolidine-2,4-dione and 4-(bromomethyl)pyridin-2-amine hydrobromide (1 equiv.). MS (m/z): [M+H]⁺=425.5, ¹H NMR (400 MHz, DMSO) δ (ppm): 7.84 (dd, J=10.6, 7.0 Hz, 3H), 7.67-7.56 (m, 2H), 6.52 (dd, J=5.3, 1.4 Hz, 1H), 6.45 (s, 1H), 5.87 (s, 2H), 4.47 (d, J=16.5 Hz, 1H), 4.32 (d, J=16.5 Hz, 1H), 1.93-1.68 (m, 2H), 1.37 (s, 3H), 0.68 (t, J=7.3 Hz, 3H).

### Example 32: Preparation of 5,5-dimethyl-1-((2-(((1-methyl-1H-pyrazol-4-yl)methyl)amino)pyridin-4-yl)methyl)-3-(4-((trifluoromethyl)thio)phenyl)imidazolidine-2,4-dione (Compound 301)

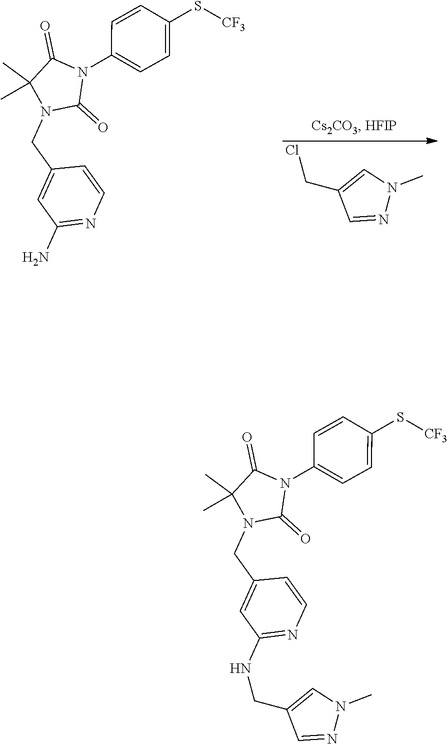

1-((2-aminopyridin-4-yl)methyl)-5,5-dimethyl-3-(4-((trifluoromethyl)thio)phenyl)imidazolidine-2,4-dione (40.0 mg, 97.5 μmol) was dissolved in HFIP (1.32 mL) and 4-(chloromethyl)-1-methyl-1H-pyrazole (25.5 mg, 195 μmol) and Cesium carbonate (96.2 mg, 292 μmol) were added and the mixture was heated at 85° C. for 12 hours. The reaction mixture was concentrated and the residue was purified by reverse phase chromatography (C18, 35%-55% 10 mM ammonium formate buffer pH 3.8/MeCN) to afford the title compound (1.9 mg, 3.6%). MS (m/z): [M+H]⁺=505.3, ¹H NMR (400 MHz, DMSO) δ (ppm): 7.91 (d, J=5.3 Hz, 1H), 7.85 (d, J=8.5 MHz, 2H), 7.71-7.60 (m, 2H), 7.54 (s, 1H), 7.31 (s, 1H), 6.62 (s, 1H), 6.49 (d, J=5.3 Hz, 1H), 6.45 (s, 1H), 4.43 (s, 2H), 4.22 (d, J=5.5 Hz, 2H), 3.73 (s, 3H), 1.37 (s, 6H).

### Example 33: Preparation of N-(4-((5,5-dimethyl-2,4-dioxo-3-(4-((trifluoromethyl)thio)phenyl)imidazolidin-1-yl)methyl)pyridin-2-yl)-2-azaspiro[3.3]heptane-6-carboxamide (Compound 302)

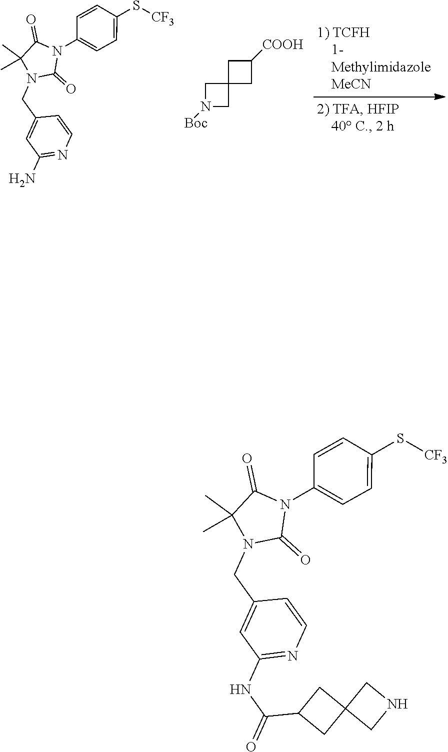 [SMILES: CC1(C)C(=O)N(c2ccc(SC(F)(F)F)cc2)C(=O)N1Cc1ccnc(N)c1 ; CC(C)(C)OC(=O)N1CC2(CC(C(=O)O)C2)C1 ; CC1(C)C(=O)N(c2ccc(SC(F)(F)F)cc2)C(=O)N1Cc1ccnc(NC(=O)C2CC3(CNC3)C2)c1]

Step 1: 1-((2-aminopyridin-4-yl)methyl)-5,5-dimethyl-3-(4-((trifluoromethyl)thio)phenyl)imidazolidine-2,4-dione (100 mg, 244 μmol), 2-(tert-Butoxycarbonyl)-2-azaspiro[3.3]heptane-6-carboxylic acid (61.9 mg, 244 μmol) and 1-Methylimidazole (80.9 μL, 975 μmol) in MeCN (6.00 mL) were stirred at room temperature for 5 mins. TCFH (138 mg, 487 μmol) was added to the suspension and the reaction heated to 40° C. for 3 h. Reaction was cooled to ambient temperature and diluted with EtOAc, washed with saturated sodium bicarbonate and brine, then dried over sodium sulfate, filtered, and concentrated in vacuo. The crude material was used as-is in step 2.

Step 2: The residue from step 1 was suspended in HFIP (3.15 mL) and TFA (18.7 μL, 244 μmol) was added. The reaction was stirred at 40° C. for 2 h and the solvent removed in vacuo. The residue was purified by reverse phase chromatography (C18, 30-50% MeCN, 10 mM Ammonium Formate pH 3.8) to afford the title compound (67.5 mg, 59%). MS (m/z): [M+H]⁺=435.2, ¹H NMR (400 MHz, DMSO) δ (ppm): 11.69 (s, 1H), 8.16 (d, J=4.9 Hz, 1H), 7.86 (d, J=8.5 Hz, 2H), 7.67 (d, J=8.5 Hz, 2H), 7.47 (d, J=3.3 Hz, 1H), 7.12 (d, J=4.9 Hz, 1H), 6.63 (d, J=3.4 Hz, 1H), 4.87 (s, 2H), 1.35 (s, 6H).

### Example 34: Preparation of 1-(4-((5,5-dimethyl-2,4-dioxo-3-(4-((trifluoromethyl)thio)phenyl)imidazolidin-1-yl)methyl)pyridin-2-yl)-3-(tetrahydrofuran-3-yl)urea (Compound 303)

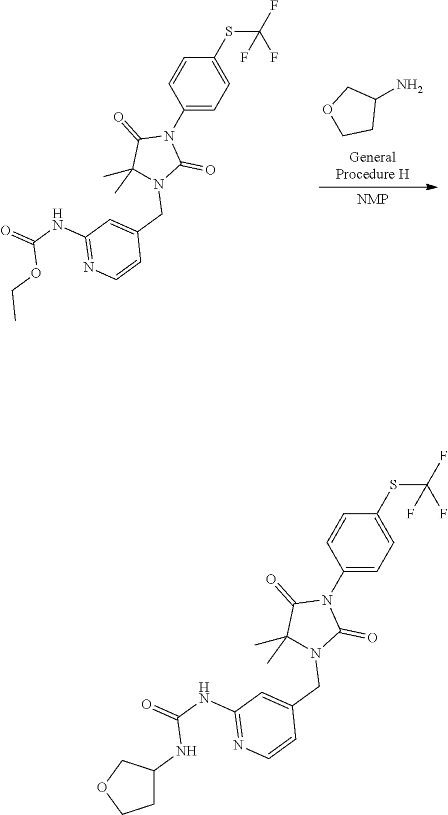 [SMILES: CCOC(=O)Nc1cc(CN2C(=O)N(c3ccc(SC(F)(F)F)cc3)C(=O)C2(C)C)ccn1 ; NC1CCOC1 ; CC1(C)C(=O)N(c2ccc(SC(F)(F)F)cc2)C(=O)N1Cc1ccnc(NC(=O)NC2CCOC2)c1]

The title compound was prepared according to the General Procedure H using excess aniline (51 equiv.). MS (m/z): [M+H]⁺=524.2. ¹H NMR (400 MHz, DMSO) δ (ppm): 9.12 (s, 1H), 8.40 (d, J=5.6 Hz, 1H), 8.11 (d, J=5.3 Hz, 1H), 7.86 (d, J=8.5 Hz, 2H), 7.65 (d, J=8.5 Hz, 2H), 7.34 (s, 1H), 6.95 (d, J=5.2 Hz, 1H), 4.57 (s, 2H), 4.33-4.17 (m, 1H), 3.86-3.63 (m, 3H), 3.50 (dd, J=8.9, 3.3 Hz, 1H), 2.21-2.08 (m, 1H), 1.80-1.65 (m, 1H), 1.40 (s, 6H).

### Example 35: Preparation of 5,5-dimethyl-1-((2-((tetrahydro-2H-pyran-4-yl)amino)pyridin-4-yl)methyl)-3-(4-((trifluoromethyl)thio)phenyl)imidazolidine-2,4-dione (Compound 310)

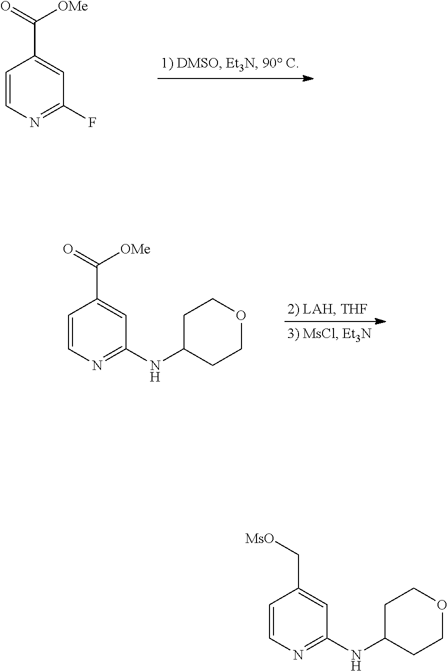 [SMILES: COC(=O)c1ccnc(F)c1 ; COC(=O)c1ccnc(NC2CCOCC2)c1 ; CS(=O)(=O)OCc1ccnc(NC2CCOCC2)c1]

Step 1: In a vial, methyl-2-fluoroisonicotinate (220 mg, 1.42 mmol), triethylamine (1 mL, 7.09 mmol) and 4-aminotetrahydropyran (296 mg, 2.84 mmol) were dissolved in DMSO (7.09 mL). The vial was sealed and heated at 100° C. for 4 hours under microwave irradiation. The reaction was added to a separatory funnel with water and DCM. The layer were separated and the aqueous phase was extracted two more time with DCM. The organic layer were combined, dried over Na2SO4, filtered and evaporated. Silica and DCM were added and the mixture was concentrated under reduce pressure. The residue was purified by silica gel chromatography eluting with a gradient of EtOAc (10 to 100%) in Heptane (100%). Desired fractions were combined and evaporated to afford methyl 2-((tetrahydro-2H-pyran-4-yl)amino)isonicotinate (250 mg, 75% yield) as a clear oil. MS (m/z): [M+H]+=237.2.

Step 2: In a flame-dried flask, methyl 2-((tetrahydro-2H-pyran-4-yl)amino)isonicotinate (200 mg, 0.83 mmol) was dissolved in THF (4.1 mL) and cooled down to −40° C. Lithium aluminum hydride (0.83 mL, 1.66 mmol) (2 M in THF) was added dropwise and the solution was stirred at −40° C. for 2 hours. The reaction was quenched with water (2 mL), NaOH 1M (2 mL) was added and some more water (2 mL). The reaction was allowed to warm-up to room temperature and stirred for another 30 minutes. The suspension was filtered on Celite and washed with EtOAc. The organic phase was separated and evaporated to afford a yellow gum. MS (m/z): [M+H]+=209.2.

Step 3: The crude product from step 2 was dissolved in DCM (4.1 mL) and cooled down to 0° C. Triethylamine (186 mL, 1.33 mmol) was added, followed by dropwise addition of methanesulfonyl chloride (97 mL, 1.24 mmol). The mixture was stirred at 0° C. for 2 hours. EtOAc was then added to crash out the triethylamine salt. The suspension was filtered on a short silica pad and washed with EtOAc. The filtrate was evaporated to afford (2-((tetrahydro-2H-pyran-4-yl)amino)pyridin-4-yl)methyl methanesulfonate (150 mg, 63% yield). MS (m/z): [M+H]+=287.1.

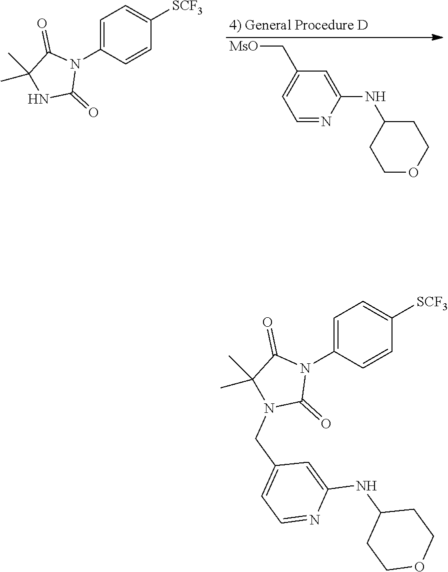

Step 4: The title compound was prepared according to the General Procedure D using (2-((tetrahydro-2H-pyran-4-yl)amino)pyridin-4-yl)methyl methanesulfonate (1.2 equiv.). [M+H]⁺=495.4, ¹H NMR (400 MHz, DMSO) δ (ppm): 7.88-7.84 (m, 3H), 7.69-7.56 (m, 2H), 6.55-6.33 (m, 3H), 4.42 (s, 2H), 3.93-3.73 (m, 3H), 3.41-3.34 (m, 2H), 1.85-1.82 (m, 2H), 1.45-1.26 (m, 8H).

### Example 36: Preparation of 1-((2-(sec-butylamino)pyridin-4-yl)methyl)-5,5-dimethyl-3-(4-((trifluoromethyl)thio)phenyl)imidazolidine-2,4-dione (Compound 381)

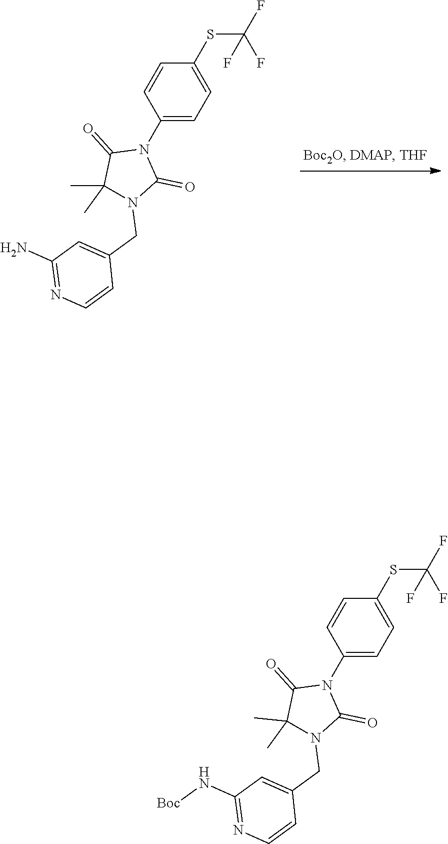 [SMILES: CC1(C)C(=O)N(c2ccc(SC(F)(F)F)cc2)C(=O)N1Cc1ccnc(N)c1 ; CC(C)(C)OC(=O)Nc1cc(CN2C(=O)N(c3ccc(SC(F)(F)F)cc3)C(=O)C2(C)C)ccn1]

Step 1: tert-butyl (4-((5,5-dimethyl-2,4-dioxo-3-(4-((trifluoromethyl)thio)phenyl)imidazolidin-1-yl)methyl)pyridin-2-yl)carbamate: 1-((2-aminopyridin-4-yl)methyl)-5,5-dimethyl-3-(4-((trifluoromethyl)thio)phenyl)imidazolidine-2,4-dione, Boc-anhydride (76.6 mg, 351 μmol), and 4-Dimethylaminopyridine (7.22 mg, 58.5 μmol) were stirred in THF (4.00 mL) at room temperature for 3 h. Crude product purified by chromatography (SiO₂, Heptanes/EtOAc: 2%-50%) to afford the title compound (110 mg, 74%). MS (m/z): [M+H]⁺=411.

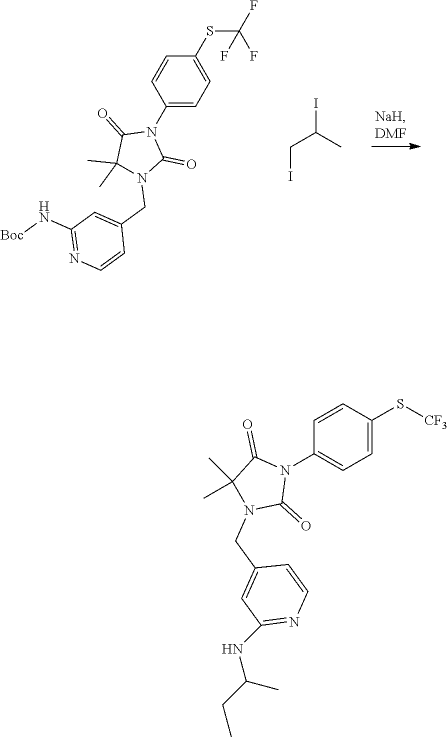

Step 2: 1-((2-sec-butylamino)pyridin-4-yl)methyl)-5,5-dimethyl-3-(4-((trifluoromethyl)thio)phenyl)imidazolidine-2.4-dione: To the compound from Step 1 was stirred in DMF (1.36 mL) at room temperature and NaH (60% dispersion, 7.8 mg, 196 μmol) was added. After 10 mins of stirring 2-iodobutane (57.5 μL, 490 μmol) was added. The reaction was heated to 40° C. overnight. The reaction was cooled to room temperature and diluted with EtOAc, washed with sat. NH₄Cl and brine. The organic portion was dried over sodium sulfate, filtered, and concentrated in vacuo. The residue was purified by reverse phase chromatography (C18, 45-65% 10 mM ammonium bicarbonate in MeCN). The appropriate fractions were combined and lyophilized affording the title compound (3.2 mg, 7.0%). MS (m/z): [M+H]⁺=467.6, 1H NMR (400 MHz, DMSO) δ (ppm): 7.89-7.80 (m, 3H), 7.69-7.61 (m, 2H), 6.46-6.36 (m, 2H), 6.20 (d, J=8.0 Hz, 1H), 4.41 (s, 2H), 3.89-3.72 (m, 1H), 1.56-1.41 (m, 2H), 1.39 (s, 6H), 1.06 (d, J=6.5 Hz, 3H), 0.84 (t, J=7.4 Hz, 3H).

### Example 37: Preparation of 1-((2-amino-3-bromopyridin-4-yl)methyl)-5,5-dimethyl-3-(4-((trifluoromethyl)thio)phenyl)imidazolidine-2,4-dione (Compound 388)

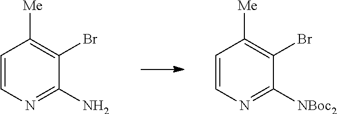

Step 1: To 2-amino-3-bromo-4-picoline (2.00 g, 10.4 mmol) in THF (34.6 mL) was added triethylamine (4.36 mL, 31.1 mmol), 4-dimethylaminopyridine (129 mg, 1.04 mmol), and di-tert-butyl dicarbonate (5.96 mL, 25.9 mmol). The reaction was stirred 2 days then diluted with DCM (50 mL), washed with half saturated NH₄Cl (50 mL), dried over MgSO₄, filtered, and concentrated to afford bis-Boc protected 3-bromo-4-methylpyridin-2-amine (4.34 g, 94%) as a red viscous oil. MS (m/z): no ionization. ¹H NMR (400 MHz, CDCl₃) δ (ppm): 8.30 (d, J=4.9 Hz, 1H), 7.13 (dd, J=4.9, 0.5 Hz, 1H), 2.47 (s, 3H), 1.41 (s, 18H).

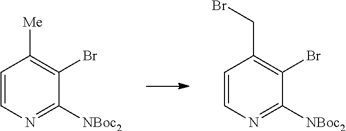

Step 2: To step-1 product (1.00 g, 2.58 mmol) in CCl₄ (12.9 mL) was added, n-bromosuccinimide (928 mg, 5.16 mmol), followed by AIBN (8.28 mg, 516 μmol). The reaction mixture was then heated to 85° C. for 36 h. The reaction mixture was diluted with DCM (50 mL) and washed with water (3×25 mL). Combined aqueous layer extracted with DCM (25 mL), combined organics dried over MgSO₄, filtered, and purified by chromatography (SiO₂, 0-100% ethyl acetate in hexanes) to afford the corresponding brominated compound (570 mg, 30%) as a brown foam. MS (m/z): no ionization. ¹H NMR (400 MHz, CDCl₃) δ (ppm): 8.42 (d, J=4.9 Hz, 1H), 7.34 (d, J=4.9 Hz, 1H), 4.55 (s, 2H), 1.40 (s, 18H).

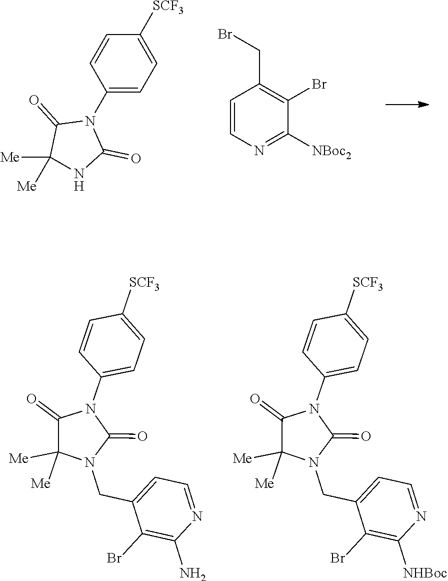

Step 3: To 5,5-dimethyl-3-(4-((trifluoromethyl)thio)phenyl)imidazolidine-2,4-dione (1.08 g, 3.54 mmol) in DMF (8.85 mL) was added NaH (60%, 425 mg, 10.6 mmol) and the reaction was stirred at room temperature for 15 min. Then a solution of above step-2 product (2.20 g, 3.54 mmol) in DMF (2.95 mL) was then added dropwise, stirring continued for 30 min. The reaction mixture was quenched with a few drops of 4M HCl in MeOH, stirred for 5 min, volatiles removed under reduced pressure, and the resulting residue was purified by reverse phase chromatography (C18 column, 5-100% ACN in 10 mM ammonium bicarbonate) to afford the title compound (700 mg, 38%) as an off-white powder. MS (m/z): [M+H]⁺=489.0. ¹H NMR (400 MHz, DMSO) δ (ppm): 7.90-7.84 (m, 3H), 7.70-7.65 (m, 2H), 6.68 (d, J=5.1 Hz, 1H), 6.27 (s, 2H), 4.50 (s, 2H), 1.44 (s, 6H).

Along with this, tert-butyl (3-bromo-4-((5,5-dimethyl-2,4-dioxo-3-(4-((trifluoromethyl)thio)phenyl)imidazolidin-1-yl)methyl)pyridin-2-yl)carbamate was also isolated as a by-product (400 mg, 18%) as an off-white powder. MS (m/z): [M−H+2]⁺=589.2. ¹H NMR (4(0) MHz, CDCl₃) δ (ppm): 8.38 (d, J=5.0 Hz, 1H), 7.79-7.74 (m, 2H), 7.66-7.61 (m, 2H), 7.41 (s, 1H), 7.02 (d, J=5.0 Hz, 1H), 4.71 (s, 2H), 1.55 (s, 9H), 1.45 (s, 6H).

### Example 38: Preparation of 1-((3-bromo-2-(isopropylamino)pyridin-4-yl)methyl)-5,5-dimethyl-3-(4-((trifluoromethyl)thio)phenyl)imidazolidine-2,4-dione (Compound 390)

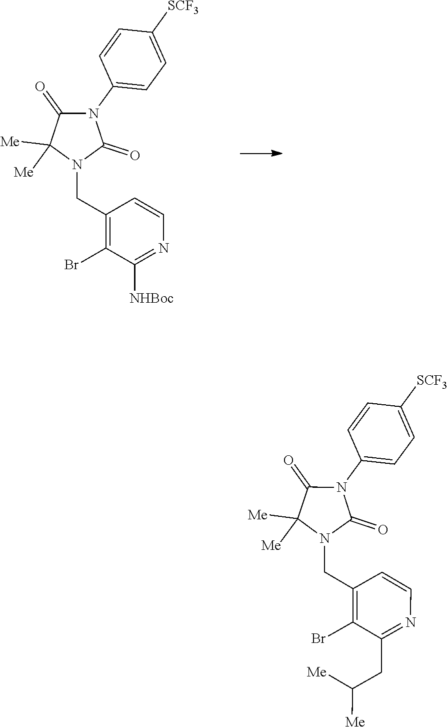

To tert-butyl (3-bromo-4-((5,5-dimethyl-2,4-dioxo-3-(4-((trifluoromethyl)thio)phenyl)imidazolidin-1-yl)methyl)pyridin-2-yl)carbamate (Example 37, 50.0 mg, 84.8 μmol) in DMF (679 μL) was charged NaH (60%, 10.2 mg, 254 μmol). The reaction mixture was stirred at room temperature for 15 min, and a solution of 2-iodopropane (42.8 μL, 424 μmol) in DMF (170 μL) was added dropwise. After addition, the reaction mixture was stirred 10 min, then heated to 85° C. for 1 h. The reaction mixture was quenched with MeOH, volatiles removed under vacuum to afford a brown oil. Which was stirred with 3M HCl in dioxane (2.18 mL, 6.54 mmol) for 1 h at 80° C. Volatiles were removed under vacuum, and the resulting residue was purified by reverse phase chromatography (C18, 5-100% ACN in 10 mM ammonium bicarbonate), to afford the title compound, HT-0002902 (11.0 mg, 24%) as a white powder. MS (m/z): [M+H]⁺=531.2. ¹H NMR (400 MHz, DMSO) δ (ppm): 7.96 (d, J=5.1 Hz, 1H), 7.89-7.84 (m, 2H), 7.70-7.64 (m, 2H), 6.68 (d, J=5.1 Hz, 1H), 5.74 (d, J=7.9 Hz, 1H), 4.50 (s, 2H), 4.26-4.15 (m, 1H), 1.44 (s, 6H), 1.19 (d, J=6.5 Hz, 6H).

### Example 39: Preparation of 1-((2-(isopropylamino)pyridin-4-yl)methyl)-5,5-dimethyl-3-(1′-(methylsulfonyl)spiro[cyclopropane-1,3′-indolin]-6′-yl)imidazolidine-2,4-dione (Compound 200)

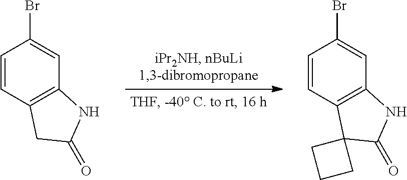 [SMILES: O=C1Cc2ccc(Br)cc2N1 ; O=C1Nc2cc(Br)ccc2C12CCC2]

Step 1: 6′-bromospiro[cyclopropane-1,3′-indolin]-2′-one: The title compound was prepared from 6-bromoindolin-2-one according to General Procedure S. MS (m/z): [M+H]⁺=240.0.

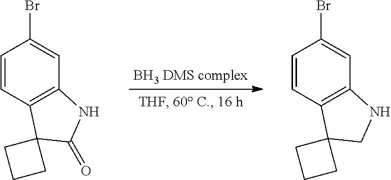 [SMILES: O=C1Nc2cc(Br)ccc2C12CCC2 ; Brc1ccc2c(c1)NCC21CCC1]

Step 2: 6′-bromospiro[cyclopropane-1,3′-indoline]: The title compound was prepared from 6′-bromospiro[cyclopropane-1,3′-indolin]-2′-one according to General Procedure N. MS (m/z): [M+H]⁺=226.1.

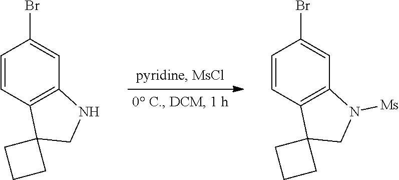 [SMILES: Brc1ccc2c(c1)NCC21CCC1 ; CS(=O)(=O)N1CC2(CCC2)c2ccc(Br)cc21]

Step 3: 6′-bromo-1′-(methylsulfonyl)spiro[cyclopropane-1,3′-indoline]: The title compound was prepared from 6′-bromospiro[cyclopropane-1,3′-indoline] according to General Procedure O. MS (m/z): [M+H]⁺: No ionization.

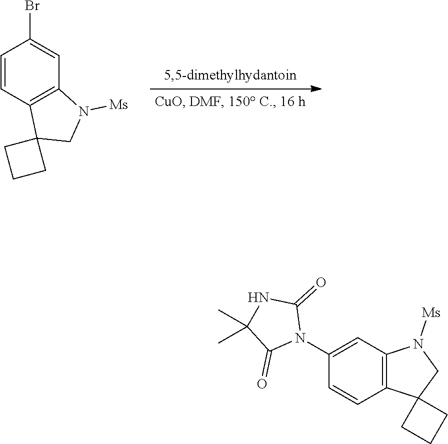 [SMILES: CS(=O)(=O)N1CC2(CCC2)c2ccc(Br)cc21 ; CC1(C)NC(=O)N(c2ccc3c(c2)N(S(C)(=O)=O)CC32CCC2)C1=O]

Step 4: 5,5-dimethyl-3-(1′-(methylsulfonyl)spiro[cyclopropane-1,3′-indolin]-6′-yl)imidazolidine-2,4-dione: The title compound was prepared from 6′-bromo-1′-(methylsulfonyl)spiro[cyclopropane-1,3′-indoline] according to General Procedure A. MS (m/z): [M+H]⁺=350.2.

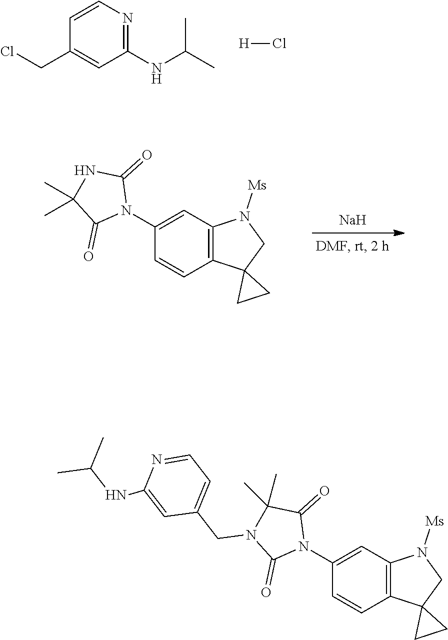

Step 5: 1-((2-(isopropylamino)pyridin-4-yl)methyl)-5,5-dimethyl-3-(1′-(methylsulfonyl)spiro[cyclopropane-1,3′-indolin]-6′-yl)imidazolidine-2,4-dione: The title compound was prepared from 5,5-dimethyl-3-(1′-(methylsulfonyl)spiro[cyclopropane-1,3′-indolin]-6′-yl)imidazolidine-2,4-dione and 4-(chloromethyl)-N-isopropylpyridin-2-amine hydrochloride according to General Procedure D. MS (m/z): [M+H]⁺=498.3.

### Example 40: Preparation of 5,5-dimethyl-1-((2-((methyl-d3)amino)pyridin-4-yl)methyl)-3-4-((trifluoromethyl)thio)phenyl)imidazolidine-2,4-dione Compound 408

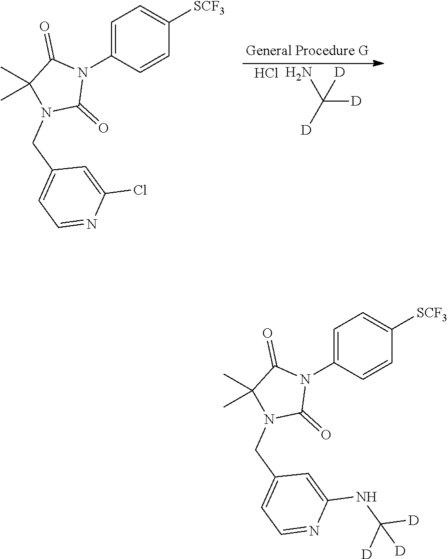

The title compound was prepared according to the General Procedure G using methyl-d3-amine hydrochloride (1.2 equiv.). MS (m/z): [M+H]⁺=460.2; ¹H NMR (400 MHz, DMSO) δ 8.29 (d, J=8.8 Hz, 2H), 8.06-7.99 (m, 21H), 7.89 (d, J=5.5 Hz, 1H), 6.57 (d, J=5.5 Hz, 1H), 6.52 (s, 1H), 4.49 (s, 2H), 1.41 (s, 6H). N−H missing.

### Example 41: Preparation of N-(5-(3-((2-(((1R,5S,6r)-3-oxabicyclo[3.1.0]hexan-6-yl)amino)pyridin-4-yl)methyl)-4,4-dimethyl-2,5-dioxoimidazolidin-1-yl)-2-(trifluoromethoxy)phenyl)-2-(dimethylamino)acetamide (Compound 281)

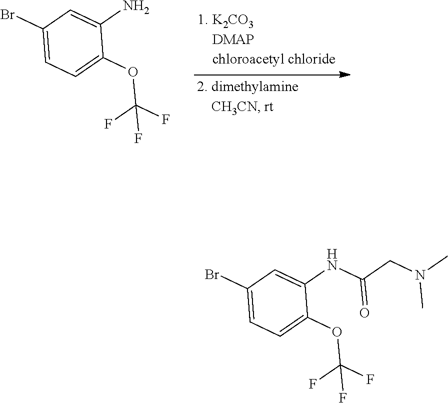 [SMILES: Nc1cc(Br)ccc1OC(F)(F)F ; CN(C)CC(=O)Nc1cc(Br)ccc1OC(F)(F)F]

Step 1: N-(5-bromo-2-(trifluoromethoxy)phenyl)-2-(dimethylamino)acetamide: To a solution of 5-Bromo-2-(trifluoromethoxy)aniline (1.00 g, 3.91 mmol) in CH₃CN (19.5 mL) was added K₂CO₃ (1.62 g, 11.7 mmol), DMAP (96.4 mg, 781 umol) and finally chloroacetyl chloride (942 uL, 11.7 mmol). The mixture was stirred at room temperature for 2 hours then dimethylamine (2.59 mL, 39.1 mmol) was added and the reaction was stirred for an additional 1 hour. The mixture was diluted with EtOAc and washed three times with water. The organic layer was dried over MgSO₄, filtered, and concentrated on silica and purified by flash chromotography using 0 to 100% DCM in hexanes to afford the title compound, 801 mg, 61%. MS (M/Z): [M+H]⁺=341.0 (M+H).

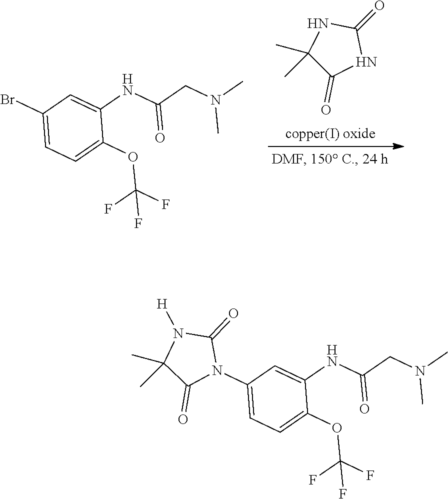

Step 2: N-(5-(4.4-dimethyl-2.5-dioxoimidazolidin-1-yl)-2-(trifluoromethoxy)phenyl)-2-(dimethylamino)acetamide: A suspension of N-(5-bromo-2-(trifluoromethoxy)phenyl)-2-(dimethylamino)acetamide (1.62 g, 4.75 mmol), Copper(I) oxide (1.05 g, 7.12 mmol) and 5,5-dimethylhydantoin (1.25 g, 9.50 mmol) was prepared at room temperature then stirred overnight at 140° C. Additional Copper(I) oxide (1.05 g, 7.12 mmol) and 5,5-dimethylhydantoin (1.25 g, 9.50 mmol) were added and the reaction was heated to 150° C. for an additional 8 hours. The reaction was cooled to room temperature and diluted with EtOAc then filtered through a plug of celite. The filtrate was washed with 5% LiCl, 10% aq NH₃, water and brine. The organic layer was dried over MgSO₄, filtered and concentrated and purified by flash column chromatography with a solvent gradient of 0 to 5% MeOH in EtOAc. Fractions containing the desired product were pooled, concentrated, then taken up in 1:1 EtOAc and heptanes. The product was extracted into 0.1 M HCl three times then the combined aqueous layers were basified with Na₂CO₃ and back extracted three times with DCM. The combined DCM layers were dried over MgSO₄, filtered, and concentrated to afford the title compound (209 mg, 11%). MS (M/Z): [M+H]⁺=389.1 (M+H).

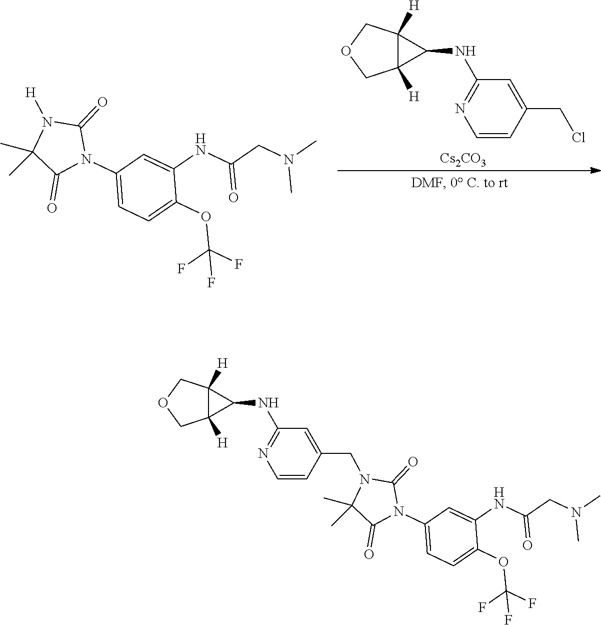

Step 3: N-(5-(3-((2-((1R,5S,6r)-3-oxabicyclo[3.1.0]hexan-6-ylamino)pyridin-4-yl)methyl)-4,4-dimethyl-2,5-dioxoimidazolidin-1-yl)-2-(trifluoromethoxy)phenyl)-2-(dimethylamino)acetamide: A solution of N-(5-(4,4-dimethyl-2,5-dioxoimidazolidin-1-yl)-2-(trifluoromethoxy)phenyl)-2-(dimethylamino)acetamide (50.0 mg, 129 umol) was cooled to 0° C., then cesium carbonate (169 mg, 515 umol) was added followed by N-((1R,5S,6r)-3-oxabicyclo[3.1.0]hexan-6-yl)-4-(chloromethyl)pyridin-2-amine (28.9 mg, 129 umol). The reaction mixture was slowly warmed to room temperature and stirred for 3 days. The reaction mixture was filtered and purified directly by reverse phase flash chromatography using 10 to 90% CH₃CN in 10 mM AMB. Fractions containing the desired product were combined and lyophilized to afford HT-0003457, 20 mg, 26%. MS (M/Z): [M+H]+=577.1 (M+H). ¹H NMR (500 MHz, MeOD) δ 8.44 (d, J=2.5 Hz, 1H), 7.93 (d, J=5.6 Hz, 1H), 7.51 (dd, J=8.8, 1.4 Hz, 1H), 7.34 (dd, J=8.8, 2.5 Hz, 1H), 6.69 (m, 2H), 4.58 (s, 2H), 4.56 (s, 1H), 4.00 (d, J=8.4 Hz, 2H), 3.73 (d, J=8.3 Hz, 2H), 3.18 (s, 2H), 2.42 (s, 6H), 2.35 (t, J=2.2 Hz, 1H), 1.82 (s, 2H), 1.47 (s, 6H).

### Example 42: Preparation of 3-(4-(2,2-difluoro-1-methylcyclopropyl)phenyl)-1-((2-(isopropylamino)pyridin-4-yl)methyl)-5,5-dimethylimidazolidine-2,4-dione (Compound 124)

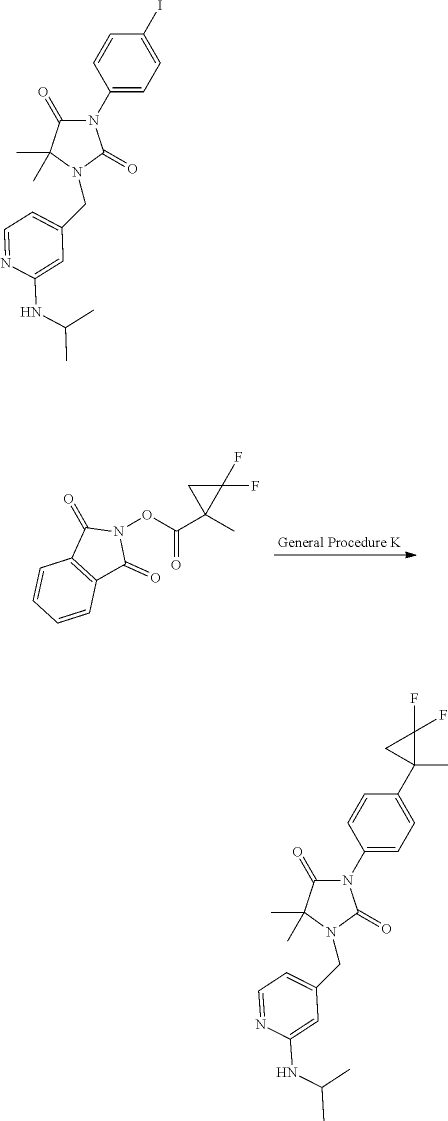 [SMILES: CC(C)Nc1cc(CN2C(=O)N(c3ccc(I)cc3)C(=O)C2(C)C)ccn1 ; CC1(C(=O)ON2C(=O)c3ccccc3C2=O)CC1(F)F ; CC(C)Nc1cc(CN2C(=O)N(c3ccc(C4(C)CC4(F)F)cc3)C(=O)C2(C)C)ccn1]

The title product was synthesized according to General Procedure K using 3-(4-iodophenyl)-1-((2-(isopropylamino)pyridin-4-yl)methyl)-5,5-dimethylimidazolidine-2,4-dione (100 mg, 0.209 mmol), 1,3-dioxoisoindolin-2-yl 2,2-difluoro-1-methylcyclopropane-1-carboxylate (118 mg, 0.418 mmol), Zinc (110 mg, 1.67 mmol), NiCl₂bpy (8.96 mg, 0.0314 mmol), chlorotrimethylsilane (81.2 uL, 0.627 mmol) in DMA. The crude material was purified by successive rounds of column chromatography 1^{st}: reverse phase chromatography on C18 column, eluting with a solvent gradient of 10-100% MeCN/H2O (0.1% of 10 mM ammonium bicarbonate buffer), 2^{nd} reverse phase chromatography on C18 column, eluting with a solvent gradient of 10-100% MeCN/H₂O (0.1% of 10 mM ammonium formate buffer), 3^{rd}: reverse phase chromatography on C18 column, eluting with a solvent gradient of 10-100% MeCN/H₂O (0.1% of 10 mM ammonium bicarbonate buffer) to afford the title product (1.98 mg, 2.1%). (1.00 mL) to afford 1.98 mg (2.1% yield) of the title product. LCMS MS (m/z). [M+H]⁺=443.4. ¹H NMR (400 MHz, DMSO) δ 7.87 (d, J=5.3 Hz, 1H), 7.47 (d, J=8.6 Hz, 2H), 7.40 (d, J=8.6 Hz, 2H), 6.41 (d, J=5.3 Hz, 1H), 6.38 (s, 1H), 6.27 (d, J=7.6 Hz, 1H), 4.40 (s, 2H), 3.99-3.89 (m, 1H), 2.01-1.91 (m, 1H), 1.69-1.59 (m, 1H), 1.49 (s, 3H), 1.37 (s, 6H), 1.10 (d, J=6.4 Hz, 6H).

### Example 43: Preparation of 3-(4-(bicyclo[1.1.1]pentan-1-yl)phenyl)-1-((2-(isopropylamino)pyridin-4-yl)methyl)-5,5-dimethylimidazolidine-2,4-dione (Compound 117)

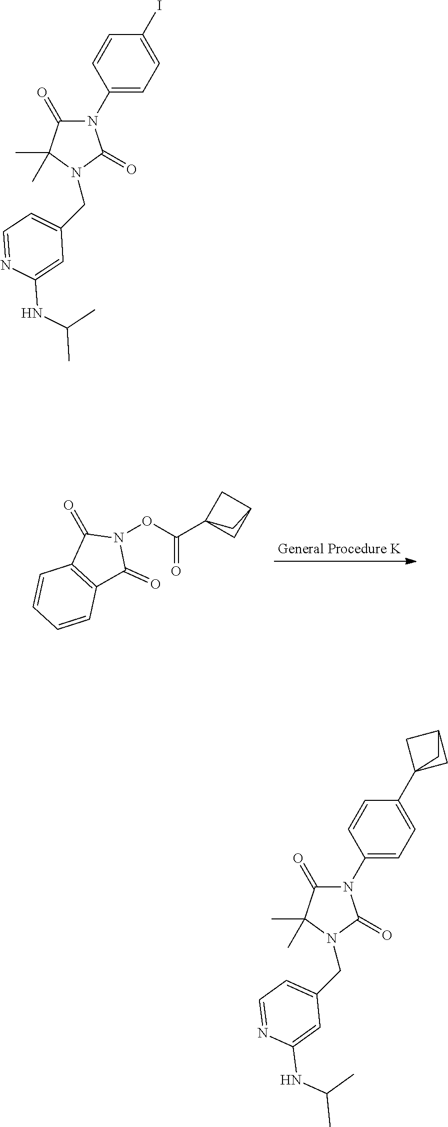 [SMILES: CC(C)Nc1cc(CN2C(=O)N(c3ccc(I)cc3)C(=O)C2(C)C)ccn1 ; O=C1c2ccccc2C(=O)N1OC(=O)C12CC(C1)C2 ; CC(C)Nc1cc(CN2C(=O)N(c3ccc(C45CC(C4)C5)cc3)C(=O)C2(C)C)ccn1]

The title product was synthesized according to General Procedure K using 3-(4-iodophenyl)-1-((2-(isopropylamino)pyridin-4-yl)methyl)-5,5-dimethylimidazolidine-2,4-dione (100 mg, 0.209 mmol), 1,3-dioxoisoindolin-2-yl bicyclo[1.1.1]pentane-1-carboxylate (108 mg, 0.418 mmol), Zinc (110 mg, 1.67 mmol), NiCl2bpy (8.96 mg, 0.0314 mmol), chlorotrimethylsilane (81.2 uL, 0.627 mmol) in DMA (1.00 mL). The crude material was purified by successive rounds of column chromatography 1^{st}: reverse phase chromatography on C18 column, eluting with a solvent gradient of 10-100% MeCN/H₂O (0.1% of 10 mM ammonium bicarbonate buffer), 2^{nd} column chromatography on silica gel, eluting with a solvent gradient of 20-70% EtOAc/heptanes to afford 32.26 mg (37% yield) of the title product. LCMS MS (m/z): [M+H]⁺: 419.3. H NMR (400 MHz, DMSO) δ 7.87 (d, J=5.4 Hz, 1H), 7.36-7.27 (m, 411), 6.48-6.38 (m, 2H), 4.41 (s, 2H), 3.99-3.87 (m, 1.1), 2.55 (s, 1H), 2.116 (s, 6H), 1.37 (s, 6H), 1.11 (d, J=6.4 Hz, 6H).

### Example 44: Preparation of 1-((2-aminopyridin-4-yl)methyl)-5,5-dimethyl-3-(3-(trifluoromethyl)-1H-indazol-6-yl)imidazolidine-2,4-dione (Compound 254)

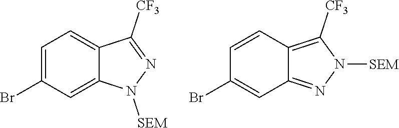 [SMILES: C[Si](C)(C)CCOCn1nc(C(F)(F)F)c2ccc(Br)cc21 ; C[Si](C)(C)CCOCn1nc2cc(Br)ccc2c1C(F)(F)F]

Step 1: Mixture of 6-bromo-3-(trifluoromethyl)-1-((2-(trimethylsilyl)ethoxy)methyl)-1H-indazole and 6-bromo-3-(trifluoromethyl)-2-((2-(trimethylsilyl)ethoxy)methyl)-2H-indazole: To a solution of 6-Bromo-3-(trifluoromethyl)-1H-indazole (500 mg, 1.89 mmol) in DMF (5.0 mL) was added sodium hydride (113 mg, 2.83 mmol) portionwise. The mixture was stirred at rt for 30 min. Then, SEMCl (0.40 mL, 2.26 mmol) was added dropwise. The reaction was stirred at rt for 30 min before quenching with water. The mixture was partitioned between water (30 mL) and EtOAc (30 mL). The aqueous layer was extracted with EtOAc (3×30 mL). The combined organic layer were washed with water (3×30 mL), brine, dried (Na₂SO₄) and concentrated. The crude product was purified by flash chromatography (Hex/EtOAc 85:15) to afford the title product as a mixture of isomers (631 mg, 85% yield).

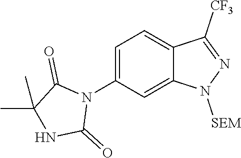 [SMILES: CC1(C)NC(=O)N(c2ccc3c(C(F)(F)F)nn(COCC[Si](C)(C)C)c3c2)C1=O]

Step 2: 5,5-dimethyl-3-(3-(trifluoromethyl)-1-((2-(trimethylsilyl)ethoxy)methyl)-1H-indazol-6-yl)imidazolidine-2,4-dione: The title compound was prepared according to General Procedure A, followed by purification by flash chromatography, starting from 5,5-dimethylhydantoin and the product from step 1. MS (m/z): [M+H]⁺=441.1.

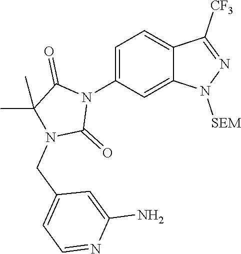 [SMILES: CC1(C)C(=O)N(c2ccc3c(C(F)(F)F)nn(COCC[Si](C)(C)C)c3c2)C(=O)N1Cc1ccnc(N)c1]

Step 3: 1-((2-aminopyridin-4-yl)methyl)-5,5-dimethyl-3-(3-(trifluoromethyl)-1-((2-(trimethylsilyl)ethoxy)methyl)-1H-indazol-6-yl)imidazolidine-2,4-dione: The title compound was prepared according to General Procedure D, starting from 5,5-dimethyl-3-(3-(trifluoromethyl)-1-((2-(trimethylsilyl)ethoxy)methyl)-1H-indazol-6-yl)imidazolidine-2,4-dione and 4-(bromomethyl)pyridine-2-amine hydrobromide. MS (m/z): [M+H]⁺=549.2.

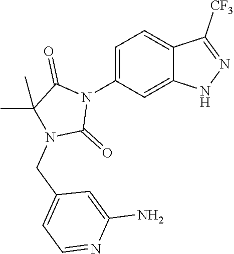 [SMILES: CC1(C)C(=O)N(c2ccc3c(C(F)(F)F)n[nH]c3c2)C(=O)N1Cc1ccnc(N)c1]

Step 4: 1-((2-aminopyridin-4-yl)methyl)-5.5-dimethyl-3-(3-(trifluoromethyl)-1H-indazol-6-yl)imidazolidine-2,4-dione: To a solution of 1-((2-aminopyridin-4-yl)methyl)-5,5-dimethyl-3-(3-(trifluoromethyl)-1-((2-(trimethylsilyl)ethoxy)methyl)-1H-indazol-6-yl)imidazolidine-2,4-dione (310 mg, 565 umol) in EtOH (4.00 mL) was added HCl 3 M aq. (4.0 mL). The mixture was heated in a screw-cap tube at 82° C. for 5 h. EtOH was removed under reduced pressure and water (10 mL) was added to the residue, which was neutralized with NH₄OH. EtOAc (20 mL) was added and the layers were separated. The aqueous layer was extracted with EtOAc (3×10 mL). The combined organic layers were washed with brine, dried over Na₂SO₄ and concentrated. Purification by reversed phase flash chromatography (25 to 45% MeCN in 10 mM AmB) followed by lyophilization to afford the title product. MS (m/z): [M+H]⁺=419.4.
The following compounds were prepared similarly to the examples above substituting the appropriate reagents and intermediates. Some examples may require additional functional group transformations or deprotection to arrive at the final compounds.

TABLE A

| Cmpd # | MS (m/z) [M + H]⁺ | ¹H NMR |
|---|---|---|
| 1 | 435.2 | ¹H NMR (400 MHz, DMSO) δ (ppm): 11.70 (s, 1H), 8.16 (d, J = 4.9 Hz, |
| | | 1H), 7.86 (d, J = 8.5 Hz, 2H), 7.67 (d, J = 8.6 Hz, 2H), 7.47 (d, J = 3.4 Hz, |
| | | 1H), 7.12 (d, J = 4.9 Hz, 1H), 6.63 (d, J = 3.4 Hz, 1H), 4.87 (s, 2H), 1.35 |
| | | (s, 6H) |
| 2 | 436.2 | ¹H NMR (400 MHz, DMSO) δ (ppm): 13.00 (2 × singlets, 1H), 8.44 (s, |
| | | 1H), 8.28 (s, 1H), 7.85 (d, J = 8.5 Hz, 2H), 7.72-7.63 (m, 2H), 7.29 (d, J = |
| | | 4.9 Hz, 1H), 4.98 (2 × singlets, 2H), 1.41 (s, 6H) |
| 3 | 436.3 | ¹H NMR (400 MHz, DMSO-d6) δ (ppm): 8.70 (s, 1H), 7.88-7.83 (m, |
| | | 2H), 7.68-7.64 (m, 2H), 7.57 (d, J = 3.5 Hz, 1H), 6.74 (d, J = 3.5 Hz, |
| | | 1H), 4.97 (s, 2H), 1.40 (s, 6H). |
| 4 | 391.7 | ¹H NMR (400 MHz, DMSO) δ (ppm): 11.69 (s, 1H), 8.17 (d, J = 4.9 Hz, |
| | | 1H), 7.53-7.46 (m, 3H), 7.38-7.31 (m, 2H), 7.09 (d, J = 4.9 Hz, 1H), |
| | | 6.62 (dd, J = 3.4, 1.4 Hz, 1H), 4.86 (s, 2H), 1.32 (s, 6H), 1.30 (s, 9H). |
| 5 | 469.2 | ¹H NMR (400 MHz, DMSO) δ (ppm): 12.05 (s, 1H), 8.21 (d, J = 4.9 Hz, |
| | | 1H), 7.86 (d, J = 8.5 Hz, 2H), 7.75-7.63 (m, 3H), 7.19 (d, J = 4.9 Hz, |
| | | 1H), 5.16 (s, 2H), 1.44 (s, 6H). |
| 6 | 425.3 | ¹H NMR (400 MHz, DMSO) δ (ppm): 12.05 (br s, 1H), 8.22 (d, J = 4.9 |
| | | Hz, 1H), 7.67 (s, 1H), 7.54-7.47 (m, 2H), 7.39-7.32 (m, 2H), 7.14 (d, J = |
| | | 4.9 Hz, 1H), 5.14 (s, 2H), 1.41 (s, 6H), 1.30 (s, 9H). |
| 7 | 443.2 | ¹H NMR (400 MHz, DMSO) δ (ppm): 11.69 (s, 1H), 8.16 (d, J = 4.9 Hz, |
| | | 1H), 7.58 (d, J = 8.4 Hz, 2H), 7.52-7.42 (m, 3H), 7.10 (d, J = 4.9 Hz, 1H), |
| | | 6.62 (d, J = 3.4 Hz, 1H), 4.86 (s, 2H), 1.39-1.30 (m, 8H), 1.19-1.14 (m, 2H). |
| 8 | 521.1/523.0 | ¹H NMR (400 MHz, DMSO) δ (ppm): 8.21 (d, J = 4.9 Hz, 1H), 7.71 (s, |
| | | 1H), 7.58 (d, J = 8.4 Hz, 2H), 7.49 (d, J = 8.5 Hz, 2H), 7.15 (d, J = 4.9 Hz, |
| | | 1H), 5.21 (s, 2H), 1.42 (s, 6H), 1.38-1.31 (m, 2H), 1.20-1.13 (m, 2H). |
| 9 | 457.3 | ¹H NMR (400 MHz, DMSO) δ (ppm): 11.49 (s, 1H), 8.03 (d, J = 4.9 Hz, |
| | | 1H), 7.58 (d, J = 8.4 Hz, 2H), 7.45 (d, J = 8.5 Hz, 2H), 7.01 (d, J = 4.9 Hz, |
| | | 1H), 6.29 (s, 1H), 4.77 (s, 2H), 2.38 (s, 3H), 1.39-1.32 (m, 2H), 1.30 (s, |
| | | 6H), 1.18-1.10 (m, 2H). |
| 10 | 491.2 | ¹H NMR (400 MHz, DMSO) δ (ppm): 11.96 (s, 1H), 8.11 (d, J = 5.0 Hz, |
| | | 1H), 7.58 (d, J = 8.4 Hz, 2H), 7.53-7.44 (m, 2H), 7.10 (d, J = 5.0 Hz, |
| | | 1H), 5.11 (s, 2H), 2.37 (s, 3H), 1.45-1.33 (m, 8H), 1.19-1.12 (m, 2H). |
| 11 | 519.45 | ¹H NMR (400 MHz, DMSO) δ (ppm): 12.22 (s, 1H), 8.17 (d, J = 4.9 Hz, |
| | | 1H), 7.93 (d, J = 7.3 Hz, 2H), 7.59 (d, J = 8.4 Hz, 2H), 7.53-7.43 (m, |
| | | 4H), 7.34 (t, J = 7.4 Hz, 1H), 7.17-7.08 (m, 2H), 4.89 (s, 2H), 1.42- |
| | | 1.33 (m, 8H), 1.20-1.13 (m, 2H). |
| 12 | 451.3 | ¹H NMR (400 MHz, DMSO) δ (ppm): 10.96 (s, 1H), 8.00 (d, J = 5.5 Hz, |
| | | 1H), 7.85 (d, J = 8.5 Hz, 2H), 7.70-7.62 (m, 2H), 6.99 (d, J = 5.5 Hz, |
| | | 1H), 4.52 (s, 2H), 3.64 (s, 2H), 1.39 (s, 6H). |
| 13 | 459.2 | ¹H NMR (400 MHz, DMSO) δ (ppm): 10.99 (s, 1H), 8.00 (d, J = 5.5 Hz, |
| | | 1H), 7.57 (d, J = 8.4 Hz, 2H), 7.51-7.42 (m, 2H), 6.96 (d, J = 5.5 Hz, |
| | | 1H), 4.51 (s, 2H), 3.62 (s, 2H), 1.41-1.33 (m, 8H), 1.15 (d, J = 5.5 Hz, 2H). |
| 14 | 465.1 | ¹H NMR (400 MHz, DMSO) δ 7.83 (d, J = 8.4 Hz, 2H), 7.74 (s, 1H), 7.63 |
| | | (d, J = 8.5 Hz, 2H), 7.28 (s, 1H), 6.49 (s, 1H), 4.37 (s, 2H), 2.71 (s, 2H), |
| | | 1.36 (s, 6H), 1.20 (s, 6H). |
| 15 | 475.2 | ¹H NMR (400 MHz, DMSO) δ 11.43 (s, 1H), 8.16 (d, J = 2.0 Hz, 1H), |
| | | 7.88-7.78 (m, 3H), 7.71-7.59 (m, 2H), 6.05 (d, J = 1.9 Hz, 1H), 4.64 (s, |
| | | 2H), 2.04-1.94 (m, 1H), 1.35 (s, 6H), 1.00-0.91 (m, 2H), 0.85-0.77 |
| | | (m, 2H). |
| 16 | 437.2 | 1H NMR (400 MHz, DMSO) δ (ppm): 7.89-7.80 (m, 2H), 7.69-7.60 |
| | | (m, 3H), 6.43 (d, J = 5.5 Hz, 1H), 6.33 (s, 1H), 4.40 (s, 2H), 3.46 (t, J = |
| | | 8.6 Hz, 2H), 3.01 (t, J = 8.4 Hz, 2H), 1.38 (s, 6H). |
| 17 | 461.4 | ¹H NMR (500 MHz, DMSO) δ 7.58 (d, J = 8.4 Hz, 2H), 7.51 (d, J = 5.2 |
| | | Hz, 1H), 7.48-7.43 (m, 2H), 6.68 (s, 1H), 6.52 (d, J = 5.2 Hz, 1H), 4.45 |
| | | (s, 2H), 4.22-4.14 (m, 2H), 3.41 (dd, J = 7.0, 4.3 Hz, 2H), 1.38 (s, 6H), |
| | | 1.37-1.31 (m, 2H), 1.20-1.15 (m, 2H). |
| 18 | 459.4 | 1H NMR (500 MHz, CDCl3) δ (ppm): 7.71 (d, J = 5.2 Hz, 1H), 7.59 (d, J = |
| | | 8.4 Hz, 2H), 7.47 (d, J = 8.5 Hz, 2H), 6.45 (d, J = 5.2 Hz, 1H), 6.37 (s, |
| | | 1H), 4.43 (s, 2H), 3.26-3.20 (m, 2H), 2.69 (t, J = 6.3 Hz, 2H), 1.85- |
| | | 1.77 (m, 2H), 1.41-1.34 (m, 8H), 1.20-1.14 (m, 2H). |
| 19 | 479.2 | 1H NMR (400 MHz, DMSO) δ (ppm): 11.06 (s, 1H), 8.00 (d, J = 5.6 Hz, |
| | | 1H), 7.90-7.79 (m, 2H), 7.71-7.61 (m, 2H), 7.00 (d, J = 5.6 Hz, 1H), |
| | | 4.62 (s, 2H), 1.43 (s, 6H), 1.41 (s, 6H). |
| 20 | 465.1 | 1H NMR (400 MHz, cd3cn) δ (ppm): 7.88-7.81 (m, 2H), 7.74 (d, J = 5.5 |
| | | Hz, 1H), 7.71-7.65 (m, 2H), 6.56 (d, J = 5.5 Hz, 1H), 5.08 (s, 1H), 4.62 |
| | | (s, 2H), 3.28 (s, 2H), 1.47 (s, 6H), 1.45 (s, 6H). |
| 21 | 465.2 | 1H NMR (400 MHz, DMSO) δ (ppm): 7.84 (d, J = 8.5 Hz, 2H), 7.70- |
| | | 7.60 (m, 3H), 6.51 (s, 1H), 6.42 (d, J = 5.5 Hz, 1H), 4.37 (s, 2H), 2.80 (s, |
| | | 2H), 1.37 (s, 6H), 1.23 (s, 6H). |
| 22 | 467.2 | 1H NMR (400 MHz, DMSO) δ (ppm): 11.70 (s, 1H), 8.30 (d, J = 8.8 Hz, |
| | | 2H), 8.16 (d, J = 4.9 Hz, 1H), 8.08-8.02 (m, 2H), 7.48 (dd, J = 3.2, 2.7 |
| | | Hz, 1H), 7.15 (d, J = 4.9 Hz, 1H), 6.64 (dd, J = 3.4, 1.8 Hz, 1H), 4.88 (s, |
| | | 2H), 1.38 (s, 6H). |
| 23 | 483.1 | 1H NMR (400 MHz, DMSO) δ (ppm): 11.00 (s, 1H), 8.30 (d, J = 8.8 Hz, |
| | | 2H), 8.07-8.01 (m, 2H), 8.00 (d, J = 5.5 Hz, 1H), 7.03 (d, J = 5.5 Hz, |
| | | 1H), 4.54 (s, 2H), 3.65 (s, 2H), 1.40 (s, 6H). |
| 24 | 495.5 | 1H NMR (500 MHz, DMSO) δ (ppm): 8.34 (d, J = 5.6 Hz, 1H), 7.64- |
| | | 7.57 (m, 2H), 7.51-7.45 (m, 2H), 7.25 (d, J = 5.6 Hz, 1H), 4.69 (s, 2H), |
| | | 1.44 (s, 6H), 1.40-1.34 (m, 2H), 1.21-1.14 (m, 2H). |
| 25 | 475.1 | ¹H NMR (400 MHz, DMSO): δ 7.54 (d, J = 8.4 Hz, 2H), 7.48 (d, J = 5.2 |
| | | Hz, 1H), 7.44-7.39 (m, 2H), 6.70 (s, 1H), 6.49 (d, J = 5.2 Hz, 1H), 4.41 |
| | | (s, 2H), 4.19 (dd, J = 10.5, 1.9 Hz, 1H), 3.68 (dd, J = 10.5, 7.3 Hz, 1H), |
| | | 3.56-3.49 (m, 1H), 1.34 (d, J = 4.3 Hz, 6H), 1.33-1.31 (m, 2H), 1.16- |
| | | 1.11 (m, 2H), 1.09 (d, J = 6.4 Hz, 3H). |
| 26 | 474.7 | 1H NMR (400 MHz, DMSO): δ (ppm): 7.58 (d, J = 8.4 Hz, 2H), 7.52 (d, J = |
| | | 5.2 Hz, 1H), 7.48-7.43 (m, 2H), 6.77 (s, 1H), 6.52 (d, J = 5.2 Hz, 1H), |
| | | 4.45 (s, 2H), 4.23 (dd, J = 10.4, 2.0 Hz, 1H), 3.72 (dd, J = 10.5, 7.4 Hz, |
| | | 1H), 3.61-3.51 (m, 1H), 1.40-1.33 (m, 8H), 1.19-1.15 (m, 2H), 1.13 |
| | | (d, J = 6.4 Hz, 3H). |
| 27 | 475.3 | ¹H NMR (500 MHz, DMSO): δ 7.58 (d, J = 8.4 Hz, 2H), 7.51 (d, J = 5.2 |
| | | Hz, 1H), 7.47-7.43 (m, 2H), 6.67 (d, J = 3.0 Hz, 1H), 6.53 (d, J = 5.2 Hz, |
| | | 1H), 4.47 (d, J = 16.5 Hz, 1H), 4.43 (d, J = 17.0 Hz, 1H), 4.20-4.12 (m, |
| | | 1H), 3.44 (ddd, J = 12.2, 3.6, 2.7 Hz, 1H), 3.11-3.05 (m, 1H), 1.39- |
| | | 1.35 (m, 8H), 1.33 (d, J = 6.3 Hz, 3H), 1.17 (s, 2H). |
| 28 | 475.3 | ¹H NMR (500 MHz, DMSO): δ (ppm): 7.59 (d, J = 8.5 Hz, 2H), 7.51 (d, J = |
| | | 5.2 Hz, 1H), 7.48-7.44 (m, 2H), 6.67 (d, J = 3.0 Hz, 1H), 6.54 (d, J = |
| | | 5.2 Hz, 1H), 4.48 (d, J = 16.7 Hz, 1H), 4.44 (d, J = 16.3 Hz, 1H), 4.21- |
| | | 4.14 (m, 1H), 3.48-3.43 (m, 1H), 3.09 (dd, J = 12.3, 9.0 Hz, 1H), 1.41- |
| | | 1.35 (m, 8H), 1.34 (d, J = 6.3 Hz, 3H), 1.21-1.15 (m, 2H). |
| 29 | 418.4 | 1H NMR (400 MHz, DMSO) δ (ppm): 11.00 (s, 1H), 8.01 (d, J = 5.5 Hz, |
| | | 1H), 7.67-7.61 (m, 2H), 7.53-7.47 (m, 2H), 6.96 (d, J = 5.5 Hz, 1H), |
| | | 4.51 (s, 2H), 3.63 (s, 2H), 1.70 (s, 6H), 1.38 (s, 6H). |
| 30 | 473.3 | 1H NMR (400 MHz, DMSO) (HCl salt) δ (ppm): 11.08 (s, 1H), 8.01 (d, J = |
| | | 5.6 Hz, 1H), 7.53-7.40 (m, 4H), 6.98 (d, J = 5.6 Hz, 1H), 5.74 (s, 1H), |
| | | 4.53 (s, 2H), 3.64 (s, 2H), 2.75-2.52 (m, 4H), 2.15-1.99 (m, 1H), 1.96- |
| | | 1.81 (m, 1H), 1.38 (s, 6H). |
| 31 | 473.3 | 1H NMR (500 MHz, DMSO) δ (ppm): 8.03 (d, J = 5.1 Hz, 1H), 7.63- |
| | | 7.56 (m, 2H), 7.51-7.46 (m, 2H), 6.98 (d, J = 5.5 Hz, 1H), 4.60 (s, 2H), |
| | | 3.76 (q, J = 7.5 Hz, 1H), 1.46-1.41 (m, 6H), 1.40-1.36 (m, 5H), 1.20- |
| | | 1.16 (m, 2H). |
| 32 | 417.4 | 1H NMR (400 MHz, DMSO) δ (ppm): 10.98 (s, 1H), 8.00 (d, J = 5.4 Hz, |
| | | 1H), 7.35 (d, J = 8.3 Hz, 2H), 7.30 (d, J = 8.3 Hz, 2H), 6.95 (d, J = 5.5 Hz, |
| | | 1H), 4.50 (s, 2H), 3.62 (s, 2H), 2.54 (s, 1H), 2.06 (s, 6H), 1.37 (s, 6H). |
| 33 | 445.3 | 1H NMR (400 MHz, CD3CN) δ (ppm): 7.72 (br s, 1H), 7.64 (d, J = 8.4 |
| | | Hz, 2H), 7.52-7.45 (m, 2H), 6.54 (d, J = 4.9 Hz, 1H), 5.15 (br s, 1H), |
| | | 4.46 (s, 2H), 3.60 (t, J = 8.4 Hz, 2H), 3.10 (t, J = 8.4 Hz, 2H), 1.44-1.39 |
| | | (m, 8H), 1.21-1.15 (m, 2H). |
| 34 | 459.4 | 1H NMR (400 MHz, DMSO) δ (ppm): 7.63 (d, J = 5.5 Hz, 1H), 7.57 (d, J = |
| | | 8.4 Hz, 2H), 7.49-7.40 (m, 2H), 6.50 (s, 1H), 6.40 (d, J = 5.5 Hz, 1H), |
| | | 4.37 (s, 2H), 3.95-3.83 (m, 1H), 3.17 (dd, J = 16.4, 9.0 Hz, 1H), 2.54 |
| | | (dd, J = 16.5, 6.7 Hz, 1H), 1.42-1.31 (m, 8H), 1.21-1.12 (m, 5H). |
| 35 | 421.4 | 1H NMR (400 MHz, DMSO-D6) δ (ppm): 11.01 (br s, 1H), 8.02 (d, J = |
| | | 5.5 Hz, 1H), 7.30 (d, J = 7.9 Hz, 1H), 6.96 (d, J = 5.5 Hz, 1H), 6.91 (dd, J = |
| | | 7.9, 1.8 Hz, 1H), 6.86 (d, J = 1.7 Hz, 1H), 4.51 (s, 2H), 4.28 (s, 2H), |
| | | 3.63 (s, 2H), 1.37 (s, 6H), 1.32 (s, 6H). |
| 36 | 407.2 | 1H NMR (400 MHz, DMSO) δ (ppm): 10.99 (s, 1H), 8.01 (d, J = 5.5 Hz, |
| | | 1H), 7.52-7.47 (m, 2H), 7.37-7.30 (m, 2H), 6.94 (d, J = 5.5 Hz, 1H), |
| | | 4.51 (s, 2H), 3.62 (s, 2H), 1.37 (s, 6H), 1.29 (s, 9H). |
| 37 | 498.3 | 1H NMR (500 MHz, DMSO) δ (ppm): 8.03 (d, J = 5.5 Hz, 1H), 7.39 (d, J = |
| | | 8.0 Hz, 1H), 7.33 (d, J = 1.7 Hz, 1H), 7.13 (dd, J = 8.0, 1.8 Hz, 1H), |
| | | 6.98 (d, J = 5.5 Hz, 1H), 4.53 (s, 2H), 3.74 (s, 2H), 3.64 (s, 2H), 3.07 (s, |
| | | 3H), 1.40 (s, 6H), 1.35 (s, 6H). |
| 38 | 473.1 | 1H NMR (400 MHz, DMSO) δ (ppm): 10.41 (s, 1H), 8.06 (d, J = 5.2 Hz, |
| | | 1H), 7.59 (d, J = 8.5 Hz, 2H), 7.48 (d, J = 8.6 Hz, 2H), 7.03 (d, J = 5.2 Hz, |
| | | 1H), 4.60 (s, 2H), 2.96 (t, J = 7.6 Hz, 2H), 2.53 (d, J = 8.6 Hz, 2H) |
| | | overlaps with solvent, 1.37 (m, 8H), 1.20-1.15 (m, 2H). |
| 39 | 488.4 | 1H NMR (500 MHz, DMSO) δ (ppm): 11.00 (s, 1H), 8.03 (d, J = 5.3 Hz, |
| | | 2H), 7.37 (d, J = 8.0 Hz, 1H), 7.07 (d, J = 7.1 Hz, 1H), 6.95 (d, J = 5.4 Hz, |
| | | 1H), 4.52 (s, 2H), 4.12 (s, 2H), 3.63 (s, 2H), 1.45-1.32 (m, 12H), 0.93- |
| | | 0.83 (m, 4H). |
| 40 | 430.4 | 1H NMR (400 MHz, DMSO) δ (ppm): 11.00 (s, 1H), 8.01 (d, J = 5.5 Hz, |
| | | 1H), 7.60-7.56 (m, 2H), 7.53-7.48 (m, 2H), 6.97 (d, J = 5.5 Hz, 1H), |
| | | 4.51 (s, 2H), 3.63 (s, 2H), 2.79-2.70 (m, 2H), 2.68-2.58 (m, 2H), 2.33- |
| | | 2.20 (m, 1H), 2.07-1.94 (m, 1H), 1.38 (s, 6H). |
| 41 | 602.3 | 1H NMR (400 MHz, DMSO-D6) δ (ppm): 11.02 (s, 1H), 8.06 (d, J = 8.8 |
| | | Hz, 1H), 8.04 (d, J = 5.5 Hz, 1H), 7.97 (d, J = 1.8 Hz, 1H), 7.40 (s, 1H), |
| | | 7.38 (dd, J = 8.6, 1.9 Hz, 1H), 7.01 (d, J = 5.5 Hz, 1H), 4.55 (s, 2H), 3.97 |
| | | (s, 2H), 3.66 (s, 2H), 3.42 (s, 3H), 1.53 (s, 6H), 1.43 (s, 6H). Trace of |
| | | aliphatic impurities present. |
| 42 | 395.4 | ¹H NMR (400 MHz, DMSO) δ 11.72 (s, 1H), 8.17 (d, J = 4.9 Hz, 1H), |
| | | 7.48 (dd, J = 3.2, 2.7 Hz, 1H), 7.09 (d, J = 4.9 Hz, 1H), 6.61 (dd, J = 3.4, |
| | | 1.9 Hz, 1H), 6.28 (s, 1H), 4.86 (s, 2H), 3.60 (s, 3H), 1.35 (s, 6H), 1.23 (s, 9H). |
| 43 | 510.2 | 1H NMR (400 MHz, DMSO) δ (ppm): 11.00 (s, 1H), 8.00 (d, J = 5.5 Hz, |
| | | 1H), 7.64 (d, J = 8.1 Hz, 1H), 7.28 (d, J = 1.7 Hz, 1H), 7.17 (dd, J = 8.0, |
| | | 1.8 Hz, 1H), 6.96 (d, J = 5.5 Hz, 1H), 4.50 (s, 2H), 4.05 (s, 2H), 3.62 (s, |
| | | 2H), 3.00 (s, 3H), 2.35-2.25 (m, 4H), 2.07-1.94 (m, 2H), 1.37 (s, 6H), |
| 44 | 495 | ¹H NMR (400 MHz, DMSO): δ 12.81 (s, 1H), 7.59 (d, J = 8.5 Hz, 2H), |
| | | 7.52-7.45 (m, 3H), 6.48 (d, J = 6.5 Hz, 1H), 4.44 (s, 2H), 4.32 (s, 2H), |
| | | 1.42 (s, 6H), 1.37 (dd, J = 6.7, 5.1 Hz, 2H), 1.20-1.14 (m, 2H). |
| 45 | 496.4 | 1H NMR (400 MHz, DMSO) δ (ppm): 10.95 (s, 1H), 7.97 (d, J = 5.5 Hz, |
| | | 1H), 7.27 (d, J = 1.7 Hz, 1H), 7.03 (dd, J = 8.1, 1.8 Hz, 1H), 6.92 (dd, J = |
| | | 8.4, 6.9 Hz, 2H), 4.47 (s, 2H), 3.96 (s, 2H), 3.59 (s, 2H), 3.02 (s, 3H), 1.34 |
| | | (s, 6H), 1.13 (t, J = 5.7 Hz, 2H), 1.03 (dd, J = 6.7, 4.5 Hz, 2H). |
| 46 | 486.4 | 1H NMR (400 MHz, DMSO) δ (ppm): 10.95 (s, 1H), 7.97 (d, J = 5.5 Hz, |
| | | 1H), 7.27 (d, J = 1.7 Hz, 1H), 7.03 (dd, J = 8.1, 1.8 Hz, 1H), 6.92 (dd, J = |
| | | 8.4, 6.9 Hz, 2H), 4.47 (s, 2H), 3.96 (s, 2H), 3.59 (s, 2H), 3.02 (s, 3H), 1.34 |
| | | (s, 6H), 1.13 (t, J = 5.7 Hz, 2H), 1.03 (dd, J = 6.7, 4.5 Hz, 2H). |
| 47 | 491.0 | 1H NMR (400 MHz, DMSO) δ 11.02 (s, 1H), 8.03 (d, J = 5.5 Hz, 1H), |
| | | 6.87 (d, J = 5.5 Hz, 1H), 4.63-4.46 (m, 2H), 3.70 (s, 3H), 3.69-3.53 (m, |
| | | 2H), 1.49 (s, 3H), 1.38 (s, 3H), 1.33 (s, 9H). |
| 48 | 405.4 | 1H NMR (400 MHz, DMSO) δ (ppm): 10.99 (s, 1H), 8.00 (d, J = 5.5 Hz, |
| | | 1H), 7.31 (s, 4H), 6.94 (d, J = 5.5 Hz, 1H), 4.50 (s, 2H), 3.62 (s, 2H), 1.39 |
| | | (s, 3H), 1.36 (s, 6H), 0.89-0.82 (m, 2H), 0.82-0.74 (m, 2H). |
| 49 | 494.6 (M − H) | 1H NMR (500 MHz, DMSO) δ (ppm): 8.29 (d, J = 5.6 Hz, 1H), 7.70- |
| | | 7.64 (m, 2H), 7.50-7.46 (m, 2H), 7.16 (d, J = 4.8 Hz, 1H), 4.68 (s, 2H), |
| | | 1.59 (s, 6H), 1.44 (s, 6H). |
| 50 | 524.3 | ¹H NMR (400 MHz, DMSO-D6) δ 11.00 (br s, 1H), 8.03 (d, J = 5.5 Hz, |
| | | 1H), 7.92 (d, J = 1.6 Hz, 1H), 7.77 (d, J = 8.4 Hz, 1H), 7.45 (s, 1H), 7.38 |
| | | (dd, J = 8.4, 1.7 Hz, 1H), 7.00 (d, J = 5.5 Hz, 1H), 4.55 (s, 2H), 3.65 (s, |
| | | 2H), 3.38 (s, 3H), 2.60 (d, J = 7.0 Hz, 2H), 2.05-1.93 (m, 1H), 1.43 (s, |
| | | 6H), 0.94 (d, J = 6.6 Hz, 6H). |
| 51 | 433.4 | 1H NMR (400 MHz, DMSO-D6) δ 10.99 (s, 1H), 8.02 (d, J = 5.5 Hz, |
| | | 1H), 7.57 (d, J = 7.9 Hz, 1H), 6.98-6.93 (m, 2H), 6.83 (d, J = 1.7 Hz, |
| | | 1H), 4.61 (s, 2H), 4.51 (s, 2H), 3.63 (s, 2H), 2.41-2.24 (m, 4H), 2.10- |
| | | 1.99 (m, 1H), 1.98-1.90 (m, 1H), 1.37 (s, 6H). |
| 52 | 511.2 | 1H NMR (400 MHz, DMSO-D6) δ 11.00 (s, 1H), 8.04 (d, J = 5.5 Hz, |
| | | 1H), 7.89 (s, 1H), 7.09 (s, 1H), 6.95 (d, J = 5.5 Hz, 1H), 4.66 (s, 2H), 4.62- |
| | | 4.48 (m, 2H), 3.71-3.54 (m, 2H), 2.46-2.35 (m, 2H), 2.35-2.23 (m, |
| | | 2H), 2.11-1.99 (m, 1H), 2.00-1.86 (m, 1H), 1.42 (s, 3H), 1.38 (s, 3H). |
| 53 | 499.2 | 1H NMR (400 MHz, DMSO-D6) δ 11.00 (s, 1H), 8.04 (d, J = 5.5 Hz, |
| | | 1H), 7.65 (s, 1H), 7.12 (s, 1H), 6.95 (d, J = 5.5 Hz, 1H), 4.60-4.47 (m, |
| | | 2H), 4.33 (s, 2H), 3.71-3.55 (m, 2H), 1.42 (s, 3H), 1.38 (s, 3H), 1.35 (s, |
| | | 3H), 1.34 (s, 3H). |
| 54 | 535.1 | 1H NMR (500 MHz, DMSO) δ 10.99 (s, 1H), 9.82 (s, 1H), 8.26 (d, J = |
| | | 2.5 Hz, 1H), 8.02 (d, J = 5.5 Hz, 1H), 7.59 (dd, J = 8.8, 1.4 Hz, 1H), 7.36 |
| | | (dd, J = 8.8, 2.5 Hz, 1H), 6.99 (d, J = 5.5 Hz, 1H), 4.53 (s, 2H), 3.65 (s, |
| | | 2H), 3.13 (s, 2H), 2.33 (s, 6H), 1.40 (s, 6H). |
| 55 | 473.5 | 1H NMR (500 MHz, DMSO) δ 7.74 (d, J = 5.2 Hz, 1H), 7.59 (d, J = 8.4 |
| | | Hz, 2H), 7.51-7.44 (m, 2H), 6.47 (d, J = 5.2 Hz, 1H), 6.29 (s, 1H), 4.44 |
| | | (s, 2H), 3.46-3.36 (m, 1H), 2.77 (dt, J = 16.6, 4.8 Hz, 1H), 2.69-2.61 |
| | | (m, 1H), 1.94-1.86 (m, 1H), 1.51-1.33 (m, 9H), 1.18 (m, 5H). |
| 56 | 500 | 1H NMR (500 MHz, DMSO): δ 7.62-7.56 (m, 3H), 7.50-7.44 (m, 2H), |
| | | 7.06 (d, J = 2.8 Hz, 1H), 6.61 (d, J = 5.1 Hz, 1H), 4.49 (s, 2H), 4.21 (dd, J = |
| | | 11.0, 3.0 Hz, 1H), 4.07 (dd, J = 11.0, 5.2 Hz, 1H), 3.91-3.86 (m, 1H), |
| | | 2.89-2.80 (m, 2H), 1.39 (s, 6H), 1.39-1.36 (m, 2H), 1.20-1.16 (m, 2H). |
| 57 | 461.3 | 1H NMR (400 MHz, DMSO-D6) δ 11.00 (s, 1H), 8.02 (d, J- = 5.5 Hz, |
| | | 1H), 7.69-7.65 (m, 2H), 7.52-7.46 (m, 2H), 6.97 (d, J = 5.5 Hz, 1H), |
| | | 4.53 (s, 2H), 3.64 (s, 2H), 1.59 (s, 6H), 1.40 (s, 6H). Contains ~1% |
| | | Acetonitrile. |
| 58 | 488.3 | 1H NMR (400 MHz, DMSO): δ 7.58 (d, J = 8.4 Hz, 2H), 7.53 (d, J = 5.2 |
| | | Hz, 1H), 7.48-7.44 (m, 2H), 6.83 (s, 1H), 6.55 (d, J = 5.2 Hz, 1H), 4.48 |
| | | (s, 2H), 4.00 (s, 2H), 1.40 (s, 6H), 1.37 (dd, J = 6.8, 5.2 Hz, 2H), 1.20- |
| | | 1.15 (m, 2H), 0.87-0.80 (m, 2H), 0.79-0.74 (m, 2H). |
| 59 | 491.3 | 1H NMR (400 MHz, DMSO) δ 10.99 (s, 1H), 8.02 (d, J = 5.5 Hz, 1H), |
| | | 7.59 (t, J = 8.0 Hz, 1H), 7.50-7.24 (m, 2H), 6.91 (d, J = 5.5 Hz, 1H), |
| | | 4.53 (s, 2H), 3.61 (s, 2H), 2.74-2.55 (m, 4H), 2.08 (d, J = 8.8 Hz, 1H), |
| | | 1.96-1.84 (m, 1H), 1.40 (s, J = 6.0 Hz, 6H). |
| 60 | 499.3 | 1H NMR (400 MHz, DMSO): δ 8.30 (d, J = 8.8 Hz, 2H), 8.06-8.01 (m, |
| | | 2H), 7.52 (d, J = 5.2 Hz, 1H), 6.75 (s, 1H), 6.60 (d, J = 5.2 Hz, 1H), 4.48 |
| | | (s, 2H), 4.24 (dd, J = 10.5, 2.0 Hz, 1H), 3.72 (dd, J = 10.5, 7.4 Hz, 1H), |
| | | 3.60-3.53 (m, 1H), 1.43 (s, 3H), 1.42 (s, 3H), 1.14 (d, J = 6.4 Hz, 3H). |
| 61 | 444.3 | 1H NMR (400 MHz, DMSO) δ 10.98 (s, 1H), 8.00 (d, J = 5.5 Hz, 1H), |
| | | 7.55-7.45 (m, 4H), 6.96 (d, J = 5.5 Hz, 1H), 4.51 (s, 2H), 3.63 (s, 2H), |
| | | 1.75 (d, J = 5.7 Hz, 1H), 1.50 (d, J = 5.7 Hz, 1H), 1.45 (s, 3H), 1.38 (s, |
| | | 6H), 0.78 (s, 3H). |
| 62 | 429.3 | 1H NMR (400 MHz, DMSO) δ 10.98 (br s, 1H), 8.08-8.02 (m, 2H), 8.01 |
| | | (d, J = 5.5 Hz, 1H), 7.80-7.74 (m, 2H), 7.00 (d, J = 5.5 Hz, 1H), 4.53 (s, |
| | | 2H), 3.64 (s, 2H), 3.25 (s, 3H), 1.40 (s, 6H). |
| 63 | 465.3 | 1H NMR (400 MHz, DMSO) δ 11.00 (s, 1H), 8.14 (d, J = 8.7 Hz, 2H), |
| | | 8.02 (d, J = 5.5 Hz, 1H), 7.96 (d, J = 8.9 Hz, 2H), 7.36 (t, J = 52.0 Hz, |
| | | 1H), 7.04 (d, J = 5.5 Hz, 1H), 4.55 (s, 2H), 3.66 (s, 2H), 1.42 (s, 6H). |
| 64 | 416.3 | 1H NMR (400 MHz, DMSO) δ 10.98 (s, 1H), 8.01 (d, J = 5.5 Hz, 1H), |
| | | 7.53-7.37 (m, 4H), 6.96 (d, J = 5.5 Hz, 1H), 4.51 (s, 2H), 3.62 (s, 2H), |
| | | 1.80-1.76 (m, 2H), 1.56-1.53 (m, 2H), 1.38 (s, 6H). |
| 65 | 444.4 | 1H NMR (400 MHz, DMSO) δ 7.64 (d, J = 7.1 Hz, 1H), 7.60-7.55 (m, |
| | | 2H), 7.53-7.47 (m, 2H), 6.62 (d, J = 7.1 Hz, 1H), 4.43 (s, 2H), 3.68 (s, |
| | | 3H), 3.37 (s, 2H), 2.83-2.70 (m, 2H), 2.70-2.59 (m, 2H), 2.33-2.20 |
| | | (m, 1H), 2.06-1.95 (m, 1H), 1.40 (s, 6H). |
| 66 | 443.9 | 1H NMR (500 MHz, DMSO) δ 11.03 (s, 1H), 8.37 (d, J = 2.0 Hz, 1H), |
| | | 8.23 (d, J = 8.6 Hz, 1H), 8.17-8.11 (m, 1H), 8.03 (d, J = 5.5 Hz, 1H), |
| | | 7.05 (d, J = 5.5 Hz, 1H), 4.56 (s, 2H), 3.67 (s, 2H), 1.43 (s, 6H). |
| 67 | 470.2 | 1H NMR (500 MHz, DMSO) δ 10.99 (s, 1H), 8.03 (d, J = 5.5 Hz, 1H), |
| | | 7.38 (d, J = 8.1 Hz, 1H), 7.33 (d, J = 1.7 Hz, 1H), 7.10 (dd, J = 7.9, 1.8 |
| | | Hz, 1H), 6.98 (d, J = 5.5 Hz, 1H), 4.53 (s, 2H), 4.00 (t, J = 8.4 Hz, 2H), |
| | | 3.65 (d, J = 6.2 Hz, 2H), 3.16 (t, J = 8.4 Hz, 2H), 3.03 (s, 3H), 1.40 (s, 6H). |
| 68 | 418.4 | 1H NMR (400 MHz, DMSO): δ 11.00 (s, 1H), 8.02 (d, J = 5.5 Hz, 1H), |
| | | 7.94-7.92 (m, J = 2.2 Hz, 1H), 7.81-7.77 (m, J = 2.2 Hz, 1H), 7.71- |
| | | 7.68 (m, 1H), 7.01 (d, J = 5.5 Hz, 1H), 4.53 (s, 2H), 3.65 (s, 2H), 3.30- |
| | | 3.24 (m, 1H), 1.40 (s, 6H), 1.30 (d, J = 6.9 Hz, 6H). |
| 69 | 442.3 | 1H NMR (400 MHz, DMSO) δ 10.98 (s, 1H), 8.00 (d, J = 5.5 Hz, 1H), |
| | | 7.50-7.43 (m, 2H), 7.43-7.34 (m, 2H), 6.96 (d, J = 5.5 Hz, 1H), 4.51 (s, |
| | | 2H), 3.63 (s, 2H), 2.29 (d, J = 5.1 Hz, 1H), 2.00 (d, J = 5.1 Hz, 1H), 1.38 |
| | | (s, 6H), 1.24-1.16 (m, 3H), 0.98-0.91 (m, 1H). |
| 70 | 455.3 | 1H NMR (400 MHz, DMSO-D6) δ 11.00 (s, 1H), 8.02 (d, J = 5.5 Hz, |
| | | 1H), 7.27 (t, J- = 1.0 Hz, 1H), 7.19-7.17 (m, 2H), 6.99 (d, J = 5.5 Hz, |
| | | 1H), 4.52 (s, 2H), 3.64 (s, 2H), 1.66 (q, J = 4.9 Hz, 2H), 1.51-1.45 (m, |
| | | 2H), 1.39 (s, 6H). |
| 71 | 434.2 | 1H NMR (400 MHz, DMSO) δ 10.98 (s, 1H), 8.00 (d, J = 5.5 Hz, 1H), |
| | | 7.59 (t, J = 8.4 Hz, 1H), 7.49 (dd, J = 11.4, 2.0 Hz, 1H), 7.36 (dd, J = 8.3, |
| | | 2.1 Hz, 1H), 6.98 (d, J = 5.5 Hz, 1H), 4.51 (s, 2H), 3.63 (s, 2H), 1.72 (dd, |
| | | J = 7.6, 4.9 Hz, 2H), 1.50 (dd, J = 7.8, 5.2 Hz, 2H), 1.38 (s, 6H). |
| 72 | 505.4 | 1H NMR (400 MHz, DMSO): δ 7.58 (d, J = 8.4 Hz, 2H), 7.53 (d, J = 4.9 |
| | | Hz, 1H), 7.48-7.43 (m, 2H), 6.78 (d, J = 2.0 Hz, 1H), 6.54 (d, J = 5.1 Hz, |
| | | 1H), 4.46 (s, 2H), 4.14-4.07 (m, 2H), 3.70-3.63 (m, 1H), 3.43-3.33 |
| | | (m, 2H), 3.30 (s, 3H), 1.39-1.35 (m, 8H), 1.19-1.16 (m, 2H). |
| 74 | 520.1 | 1H NMR(500 MHz, DMSO) δ 10.99 (s, 1H), 8.02 (d, J = 5.5 Hz, 1H), |
| | | 7.45-7.41 (m, 1H), 7.26 (d, J = 2.4 Hz, 1H), 7.19 (dd, J = 8.6, 2.4 Hz, |
| | | 1H), 6.99 (d, J = 5.5 Hz, 1H), 4.52 (s, 2H), 3.77-3.70 (m, 4H), 3.65 (s, |
| | | 2H), 3.03-2.97 (m, 4H), 1.40 (s, 6H) |
| 75 | 577.2 | 1H NMR(500 MHz, DMSO) δ 11.00 (s, 1H), 9.90 (s, 1H), 8.37 (d, J = 2.4 |
| | | Hz, 1H), 8.03 (d, J = 5.5 Hz, 1H), 7.61 (d, J = 7.6 Hz, 1H), 7.36 (dd, J = |
| | | 8.9, 2.5 Hz, 1H), 7.00 (d, J = 5.5 Hz, 1H), 4.53 (s, 2H), 3.68-3.62 (m, |
| | | 6H), 3.21 (s, 2H), 2.61-2.54 (m, 4H), 1.41 (s, 6H). |
| 76 | 444.3 | 1H NMR (500 MHz, DMSO) δ 11.01 (s, 1H), 8.35 (d, J = 8.4 Hz, 1H), |
| | | 8.25 (d, J = 1.8 Hz, 1H), 8.10 (dd, J = 8.4, 1.9 Hz, 1H), 8.03 (d, J = 5.5 |
| | | Hz, 1H), 7.05 (d, J = 5.5 Hz, 1H), 4.56 (s, 2H), 3.67 (s, 2H), 1.42 (s, 6H). |
| 77 | 543.5 | 1H NMR (500 MHz, DMSO) δ 12.14 (s, 1H), 8.14 (d, J = 5.0 Hz, 1H), |
| | | 7.60 (d, J = 8.4 Hz, 2H), 7.49 (d, J = 8.5 Hz, 2H), 7.14 (d, J = 5.0 Hz, 1H), |
| | | 6.35 (s, 1H), 4.83 (s, 2H), 1.41-1.30 (m, 17H), 1.21-1.15 (m, 2H). |
| 78 | 531.2 | 1H NMR (500 MHz, DMSO): δ 8.21 (d, J = 5.4 Hz, 1H), 7.60 (d, J = 8.4 |
| | | Hz, 2H), 7.51-7.47 (m, 2H), 7.23 (d, J = 5.4 Hz, 1H), 4.58 (s, 2H), 4.38 |
| | | (q, J = 7.1 Hz, 2H), 3.90 (s, 2H), 1.40 (s, 6H), 1.37 (dd, J = 6.7, 5.2 Hz, |
| | | 2H), 1.32 (t, J = 7.1 Hz, 3H), 1.20-1.16 (m, 2H). |
| 79 | 438.4 | 1H NMR (400 MHz, DMSO-D6) δ 11.00 (s, 1H), 8.01 (d, J = 5.5 Hz, |
| | | 1H), 7.67 (d, J = 7.5 Hz, 1H), 6.99 (d, J = 5.5 Hz, 1H), 6.65 (d, J = 2.3 Hz, |
| | | 1H), 6.52 (dd, J = 7.4, 2.3 Hz, 1H), 4.51 (s, 2H), 3.81 (s, 2H), 3.64 (s, |
| | | 2H), 1.37 (s, 6H), 0.91 (s, 9H). |
| 80 | 336.4 | 1H NMR (400 MHz, DMSO) δ 8.02-7.95 (m, 2H), 7.83 (d, J = 5.2 Hz, |
| | | 1H), 7.75-7.67 (m, 2H), 6.49 (dd, J = 5.3, 1.3 Hz, 1H), 6.42 (s, 1H), 5.85 |
| | | (s, 2H), 4.43 (s, 2H), 1.39 (s, 6H). |
| 81 | 394.6 | 1H NMR (400 MHz, DMSO) δ 8.12-8.02 (m, 2H), 7.95 (d, J = 3.2 Hz, |
| | | 1H), 7.87-7.79 (m, 2H), 7.64-7.54 (m, 2H), 6.49 (dd, J = 5.3, 1.3 Hz, |
| | | 1H), 6.43 (s, 1H), 5.87 (s, 2H), 4.43 (s, 2H), 1.39 (s, 6H). |
| 82 | 376.6 | 1H NMR (400 MHz, DMSO) δ (ppm): 7.82 (d, J = 5.2 Hz, 1H), 7.44 (s, |
| | | 4H), 6.47 (dd, J = 5.3, 1.3 Hz, 1H), 6.40 (s, 1H), 5.87 (s, 2H), 4.41 (s, |
| | | 2H), 1.83-1.73 (m, 2H), 1.60-1.50 (m, 2H), 1.37 (s, 6H). |
| 83 | 419.6 | 1H NMR (400 MHz, DMSO) δ (ppm): 7.84 (d, J = 5.3 Hz, 1H), 7.59 (d, J = |
| | | 8.4 Hz, 2H), 7.51-7.42 (m, 2H), 6.49 (dd, J = 5.3, 1.4 Hz, 1H), 6.42 (s, |
| | | 1H), 5.87 (s, 2H), 4.43 (s, 2H), 1.44-1.33 (m, 8H), 1.20-1.14 (m, 2H |
| 84 | 461.4 | 1H NMR (400 MHz, DMSO) δ (ppm): 7.87 (d, J = 5.2 Hz, 1H), 7.58 (d, J = |
| | | 8.2 Hz, 2H), 7.50-7.39 (m, 2H), 6.42 (d, J = 5.2 Hz, 1H), 6.38 (s, 1H), |
| | | 6.25 (d, J = 7.4 Hz, 1H), 4.41 (s, 2H), 4.03-3.86 (m, 1H), 1.43-1.32 (m, |
| | | 8H), 1.19-1.13 (m, 2H), 1.10 (d, J = 6.4 Hz, 6H). |
| 85 | 381.6 | 1H NMR (400 MHz, DMSO) δ (ppm): 7.82 (d, J = 5.2 Hz, 1H), 7.45- |
| | | 7.30 (m, 4H), 6.47 (d, J = 4.4 Hz, 1H), 6.40 (s, 1H), 5.85 (s, 2H), 4.41 (s, |
| | | 2H), 4.03 (t, J = 7.8 Hz, 1H), 3.99-3.90 (m, 1H), 3.79 (q, J = 7.8 Hz, |
| | | 1H), 3.56 (t, J = 7.8 Hz, 1H), 3.47-3.38 (m, 1H), 2.38-2.25 (m, 1H), |
| | | 2.00-1.86 (m, 1H), 1.36 (s, 6H). |
| 86 | 391.6 | 1H NMR (400 MHz, DMSO) δ 7.82 (d, J = 5.2 Hz, 1H), 7.54-7.47 (m, |
| | | 2H), 7.39-7.33 (m, 2H), 6.47 (dd, J = 5.3, 1.4 Hz, 1H), 6.41 (s, 1H), 6.24- |
| | | 6.18 (m, 1H), 5.88 (d, J = 18.2 Hz, 2H), 4.41 (s, 2H), 2.42-2.34 (m, |
| | | 2H), 2.23-2.13 (m, 2H), 1.77-1.67 (m, 2H), 1.66-1.55 (m, 2H), 1.37 |
| | | (s, 6H). |
| 87 | 393.3 | 1H NMR (400 MHz, DMSO) δ (ppm): 7.82 (d, J = 5.2 Hz, 1H), 7.37- |
| | | 7.26 (m, 4H), 6.46 (dd, J = 5.3, 1.3 Hz, 1H), 6.40 (s, 1H), 5.86 (s, 2H), |
| | | 4.41 (s, 2H), 2.61-2.52 (m, 1H), 1.79 (d, J = 9.6 Hz, 4H), 1.49-1.25 (m, 11H). |
| 88 | 369.4 | ¹H NMR (400 MHz, DMSO) δ (ppm): 7.82 (d, J = 5.2 Hz, 1H), 7.64- |
| | | 7.49 (m, 2H), 7.40-7.20 (m, 2H), 6.46 (dd, J = 5.3, 1.3 Hz, 1H), 6.40 (s, |
| | | 1H), 5.86 (s, 2H), 5.10 (s, 1H), 4.41 (s, 2H), 1.43 (s, 6H), 1.37 (s, 6H). |
| 89 | 425.6 | 1H NMR (500 MHz, DMSO) δ (ppm): 7.90 (d, J = 5.2 Hz, 1H), 7.65 (d, J = |
| | | 8.2 Hz, 2H), 7.42 (d, J = 8.2 Hz, 2H), 6.44 (d, J = 5.3 Hz, 1H), 6.41 (s, |
| | | 1H), 6.29 (d, J = 7.6 Hz, 1H), 4.43 (s, 2H), 4.02-3.93 (m, 1H), 1.41 (s, |
| | | 6H), 1.13 (d, J = 6.4 Hz, 6H), 0.29 (s, 9H). |
| 90 | 381.2 | 1H NMR (400 MHz, DMSO) δ (ppm): 7.85 (d, J = 5.3 Hz, 1H), 7.45- |
| | | 7.32 (m, 4H), 6.50 (dd, J = 5.3, 1.5 Hz, 1H), 6.44 (s, 1H), 5.88 (s, 2H), |
| | | 4.84 (d, J = 5.7 Hz, 2H), 4.58 (d, J = 5.8 Hz, 2H), 4.44 (s, 2H), 1.66 (s, |
| | | 3H), 1.40 (s, 6H). |
| 91 | 379.1 | 1H NMR (500 MHz, DMSO) δ 7.91 (d, J = 8.5 Hz, 2H), 7.85 (d, J = 5.2 |
| | | Hz, 1H), 7.74 (d, J = 8.3 Hz, 2H), 6.52 (dd, J = 5.3, 1.4 Hz, 1H), 6.45 (s, |
| | | 1H), 5.86 (s, 2H), 4.45 (s, 1H), 1.42 (s, 6H). |
| 92 | 479.2 | 1H NMR (400 MHz, DMSO) δ 7.90-7.81 (m, 3H), 7.28-7.21 (m, 2H), |
| | | 6.42 (dd, J = 5.3, 1.3 Hz, 1H), 6.38 (s, 1H), 6.25 (d, J = 7.6 Hz, 1H), 4.39 |
| | | (s, 2H), 4.00-3.88 (m, 1H), 1.37 (s, 6H), 1.10 (d, J = 6.4 Hz, 6H). |
| 93 | 459.3 | 1H NMR (400 MHz, DMSO) δ (ppm): 7.94-7.88 (m, 1H), 7.57 (d, J = |
| | | 8.5 Hz, 2H), 7.46-7.39 (m, 2H), 6.72 (d, J = 2.2 Hz, 1H), 6.58-6.49 (m, |
| | | 2H), 4.47 (s, 2H), 1.41-1.31 (m, 8H), 1.16 (s, 2H), 0.67-0.61 (m, 2H), |
| | | 0.40-0.34 (m, 2H). |
| 94 | 393.2 | 1H NMR (500 MHz, DMSO) δ 7.85 (d, J = 5.3 Hz, 1H), 7.53-7.45 (m, |
| | | 4H), 6.50 (dd, J = 5.3, 1.3 Hz, 1H), 6.43 (s, 1H), 5.87 (s 2H), 4.44 (s, 2H), |
| | | 3.73 (q, J = 11.5 Hz, 2H), 1.40 (s, 6H). |
| 95 | 421.4 | 1H NMR (500 MHz, DMSO) δ 7.85 (d, J = 5.2 Hz, 1H), 7.68 (d, J = 8.6 |
| | | Hz, 2H), 7.50-7.45 (m, 2H), 6.50 (dd, J = 5.3, 1.5 Hz, 1H), 6.43 (d, J = |
| | | 0.6 Hz, 1H), 5.87 (s, 2H), 4.44 (s, 2H), 1.60 (s, 6H), 1.40 (s, 6H). |
| 96 | 411.4 | 1H NMR (400 MHz, DMSO) δ 7.87 (d, J = 5.2 Hz, 1H), 7.30 (d, J = 8.4 |
| | | Hz, 2H), 7.20 (d, J = 8.4 Hz, 2H), 6.41 (d, J = 5.3 Hz, 1H), 6.37 (s, 1H), |
| | | 6.27 (d, J = 7.5 Hz, 1H), 5.03-4.77 (m, 1H), 4.39 (s, 2H), 4.02-3.85 (m, |
| | | 1H), 2.56-2.51 (m, 1H), 1.62-1.45 (m, 1H), 1.36 (s, 6H), 1.23-1.13 |
| | | (m, 1H), 1.10 (d, J = 6.4 Hz, 6H). |
| 97 | 419.4 | 1H NMR (400 MHz, DMSO) δ (ppm): 7.87 (d, J = 5.2 Hz, 1H), 7.28 (d, J = |
| | | 8.3 Hz, 2H), 7.20 (d, J = 8.4 Hz, 2H), 6.41 (d, J = 5.3 Hz, 1H), 6.38 (s, |
| | | 1H), 6.28 (d, J = 7.6 Hz, 1H), 4.39 (s, 2H), 4.00-3.86 (m, 1H), 2.33 (dd, |
| | | J = 7.9, 4.4 Hz, 1H), 1.53 (dd, J = 7.9, 4.2 Hz, 1H), 1.36 (s, 6H), 1.10 (d, J = |
| | | 6.4 Hz, 6H), 1.05 (t, J = 4.3 Hz, 1H), 0.98-0.86 (m, 3H), 0.65-0.58 |
| | | (m, 1H). |
| 98 | 407.4 | 1H NMR (400 MHz, DMSO) δ (ppm): 7.87 (d, J = 5.2 Hz, 1H), 7.36- |
| | | 7.25 (m, 4H), 6.41 (dd, J = 5.3, 1.3 Hz, 1H), 6.37 (s, 1H), 6.28 (d, J = 7.6 |
| | | Hz, 1H), 4.39 (s, 2H), 3.99-3.88 (m, 1H), 1.38 (s, 3H), 1.36 (s, 6H), 1.10 |
| | | (d, J = 6.4 Hz, 6H), 0.89-0.84 (m, 2H), 0.80-0.75 (m, 2H). |
| 99 | 411.4 | 1H NMR (400 MHz, DMSO) δ 7.87 (d, J = 5.2 Hz, 1H), 7.47-7.41 (m, |
| | | 2H), 7.40-7.35 (m, 2H), 6.42 (dd, J = 5.3, 1.4 Hz, 1H), 6.38 (s, 1H), 6.29- |
| | | 6.25 (m, 1H), 4.40 (s, 2H), 4.02-3.87 (m, 1H), 1.55-1.44 (m, 2H), |
| | | 1.38 (s, 6H), 1.23-1.14 (m, 2H), 1.10 (d, J = 6.4 Hz, 6H). |
| 100 | 381.4 | 1H NMR (500 MHz, DMSO) δ 7.85 (d, J = 5.2 Hz, 1H), 7.42 (d, J = 8.5 |
| | | Hz, 2H), 7.31 (d, J = 8.5 Hz, 2H), 6.49 (d, J = 5.2 Hz, 1H), 6.43 (s, 1H), |
| | | 5.87 (s, 2H), 4.73 (t, J = 5.4 Hz, 1H), 4.43 (s, 2H), 3.56 (d, J = 4.9 Hz, |
| | | 2H), 2.56-2.46 (m, 4H), 1.39 (s, 6H), 0.90-0.82 (m, 2H), 0.80-0.76 |
| | | (m, 2H). |
| 101 | 421.4 | 1H NMR (400 MHz, DMSO) δ 7.87 (d, J = 5.2 Hz, 1H), 7.35-7.29 (m, |
| | | 2H), 7.28-7.22 (m, 2H), 6.41 (dd, J = 5.3, 1.4 Hz, 1H), 6.37 (s, 1H), 6.28 |
| | | (d, J = 7.6 Hz, 1H), 4.40 (s, 2H), 4.00-3.89 (m, 1H), 2.34-2.24 (m, 2H), |
| | | 2.16-1.97 (m, 3H), 1.82-1.72 (m, 1H), 1.42 (s, 3H), 1.37 (s, 6H), 1.10 |
| | | (d, J = 6.4 Hz, 6H). |
| 102 | 443.3 | 1H NMR (400 MHz, DMSO) δ 7.87 (d, J = 5.2 Hz, 1H), 7.45-7.40 (m, |
| | | 2H), 7.40-7.32 (m, 2H), 6.41 (dd, J = 5.3, 1.4 Hz, 1H), 6.38 (s, 1H), 6.28 |
| | | (d, J = 7.5 Hz, 1H), 4.40 (s, 2H), 4.01-3.87 (m, 1H), 3.51-3.39 (m, 1H), |
| | | 3.08-2.93 (m, 2H), 2.80-2.60 (m, 2H), 1.37 (s, 6H), 1.10 (d, J = 6.4 Hz, 6H). |
| 103 | 483.3 | 1H NMR (400 MHz, DMSO) δ 7.92 (d, J = 5.2 Hz, 1H), 7.58 (d, J = 8.4 |
| | | Hz, 2H), 7.50-7.34 (m, 2H), 6.89 (t, J = 6.1 Hz, 1H), 6.61-6.47 (m, |
| | | 2H), 6.06 (tt, J = 56.6, 4.1 Hz, 1H), 4.45 (s, 2H), 3.73-3.55 (m, 2H), 1.39- |
| | | 1.32 (m, 8H), 1.16 (br s, 2H). |
| 104 | 423.4 | 1H NMR (400 MHz, DMSO) δ (ppm): 7.87 (d, J = 5.2 Hz, 1H), 7.40- |
| | | 7.32 (m, 4H), 6.42 (dd, J = 5.3, 1.4 Hz, 1H), 6.38 (s, 1H), 6.26 (d, J = 7.6 |
| | | Hz, 1H), 4.40 (s, 2H), 3.95 (dq, J = 13.0, 6.4 Hz, 1H), 3.17 (s, 3H), 1.38 |
| | | (s, 6H), 1.20-1.15 (m, 2H), 1.10 (d, J = 6.4 Hz, 6H), 1.03-0.96 (m, 2H). |
| 105 | 491.4 | 1H NMR (500 MHz, MeOD) δ 7.88 (d, J = 5.4 Hz, 1H), 7.61 (d, J = 8.4 |
| | | Hz, 2H), 7.50-7.45 (m, 2H), 6.57 (dd, J = 5.4, 1.4 Hz, 1H), 6.55 (s, 1H), |
| | | 4.52 (s, 2H), 4.11-4.03 (m, 1H), 3.44 (dd, J = 9.4, 5.3 Hz, 1H), 3.38- |
| | | 3.33 (m, 4H), 1.46 (s, 6H), 1.42-1.38 (m, 2H), 1.20 (d, J = 6.6 Hz, 3H), |
| | | 1.15-1.12 (m, 2H). |
| 106 | 503.3 | ¹H NMR (400 MHz, DMSO) δ (ppm): 7.87 (d, J = 5.3 Hz, 1H), 7.58 (d, J = |
| | | 8.4 Hz, 2H), 7.49-7.37 (m, 2H), 6.46-6.40 (m, 3H), 4.41 (s, 2H), |
| | | 3.89-3.81 (m, 3H), 3.39-3.31 (m, 2H), 1.83 (d, J = 10.3 Hz, 2H), 1.42- |
| | | 1.31 (m, 10H), 1.16 (br s, 2H). |
| 107 | 432.3 | 1H NMR (400 MHz, DMSO) δ (ppm): 7.87 (d, J = 5.2 Hz, 1H), 7.62- |
| | | 7.54 (m, 2H), 7.52-7.45 (m, 2H), 6.42 (d, J = 5.2 Hz, 1H), 6.38 (s, 1H), |
| | | 6.24 (d, J = 7.3 Hz, 1H), 4.40 (s, 2H), 3.99-3.89 (m, 1H), 2.78-2.58 (m, |
| | | 4H), 2.32-2.22 (m, 1H), 2.05-1.95 (m, 1H), 1.38 (s, 6H), 1.09 (d, J = |
| | | 6.4 Hz, 6H). |
| 108 | 421.4 | 1H NMR (400 MHz, DMSO) δ 7.86 (d, J = 5.1 Hz, 1H), 7.33-7.27 (m, |
| | | 2H), 7.27-7.20 (m, 2H), 6.40 (d, J = 5.3 Hz, 1H), 6.37 (s, 1H), 6.25 (d, J = |
| | | 7.7 Hz, 1H), 4.39 (s, 2H), 3.99-3.88 (m, 1H), 1.90 (dd, J = 8.4, 5.9 Hz, |
| | | 1H), 1.36 (s, 6H), 1.19 (s, 3H), 1.09 (d, J = 6.4 Hz, 6H), 0.92-0.87 (m, |
| | | 1H), 0.80-0.74 (m, 4H). |
| 109 | 486.4 | 1H NMR (400 MHz, DMSO) δ (ppm): 8.04 (d, J = 5.2 Hz, 1H), 7.67- |
| | | 7.41 (m, 4H), 7.00 (s, 1H), 6.67 (d, J = 5.2 Hz, 1H), 6.58 (s, 1H), 4.49 (s, |
| | | 2H), 1.68 (s, 6H), 1.41 (s, 6H), 1.37 (s, 2H), 1.18 (s, 2H). |
| 110 | 408.3 | 1H NMR (400 MHz, DMSO-d6) δ 7.85 (d, J = 5.2 Hz, 1H), 7.49-7.44 |
| | | (m, 1H), 7.10-7.05 (m, 2H), 6.50 (dd, J = 5.3, 1.4 Hz, 1H), 6.43 (s, 1H), |
| | | 5.88 (s, 2H), 4.44 (s, 2H), 3.14 (s, 3H), 1.40 (s, 6H), 1.30 (s, 6H). |
| 111 | 450.3 | 1H NMR (400 MHz, DMSO-d6) δ 7.90 (d, J = 5.3 Hz, 1H), 7.50-7.44 |
| | | (m, 1H), 7.10-7.05 (m, 2H), 6.44 (dd, J = 5.3, 1.3 Hz, 1H), 6.41 (s, 1H), |
| | | 6.30 (d, J = 7.6 Hz, 1H)- 4.43 (s, 2H), 4.01-3.92 (m, 1H), 3.14 (s, 3H), |
| | | 1.40 (s, 6H), 1.30 (s, 6H), 1.12 (d, J = 6.4 Hz, 6H). |
| 112 | 433.4 | 1H NMR (400 MHz, DMSO) δ 7.87 (d, J = 5.3 Hz, 1H), 7.33-7.26 (m, |
| | | 4H), 6.41 (dd, J = 5.3, 1.4 Hz, 1H), 6.37 (s, 1H), 6.27 (d, J = 7.6 Hz, 1H), |
| | | 4.39 (s, 2H), 4.00-3.89 (m, 1H), 1.91 (s, 6H), 1.37 (s, 6H), 1.22 (s, 3H), |
| | | 1.10 (d, J = 6.4 Hz, 6H). |
| 113 | 531.5 | 1H NMR (400 MHz, DMSO) δ 7.89 (d, J = 5.2 Hz, 1H), 7.59 (d, J = 8.5 |
| | | Hz, 2H), 7.50-7.42 (m, 2H), 6.46 (dd, J = 5.3, 1.4 Hz, 1H), 6.40 (s, 1H), |
| | | 6.33 (d, J = 7.7 Hz, 1H), 4.43 (s, 2H), 4.12-3.96 (m, 1H), 3.69-3.52 (m, |
| | | 2H), 1.93-1.75 (m, 2H), 1.39 (s, 6H), 1.38-1.35 (m, 2H), 1.27-1.20 |
| | | (m, 2H), 1.19 (s, 3H), 1.18 (s, 2H), 1.13 (s, 3H). |
| 114 | 491.3 | 1H NMR (400 MHz, DMSO) δ (ppm): 7.91-7.88 (m, 1H), 7.60 (d, J = |
| | | 8.4 Hz, 2H), 7.50-7.45 (m, 2H), 6.48-6.45 (m, 2H), 6.30 (d, J = 7.9 Hz, |
| | | 1H), 4.44 (s, 2H), 4.14-4.07 (m, 1H), 3.39 (dd, J = 9.2, 5.1 Hz, 1H), 3.26 |
| | | (s, 3H), 3.22 (dd, J = 9.2, 6.2 Hz, 1H), 1.43-1.36 (m, 8H), 1.20-1.17 |
| | | (m, 2H), 1.11 (d, J = 6.6 Hz, 3H). |
| 115 | 475.2 | 1H NMR (400 MHz, DMSO) δ 7.87 (d, J = 5.3 Hz, 1H), 7.49-7.40 (m, |
| | | 4H), 6.42 (dd, J = 5.3, 1.4 Hz, 1H), 6.38 (s, 1H), 6.27 (d, J = 7.6 Hz, 1H), |
| | | 4.41 (s, 2H), 3.99-3.89 (m, 1H), 2.74-2.53 (m, 4H), 2.16-2.02 (m, |
| | | 1H), 1.96-1.81 (m, 1H), 1.38 (s, 6H), 1.10 (d, J = 6.4 Hz, 6H). |
| 116 | 457.2 | 1H NMR (400 MHz, DMSO) δ 7.87 (d, J = 5.2 Hz, 1H), 7.41-7.35 (m, |
| | | 4H), 6.41 (d, J = 5.3 Hz, 1H), 6.38 (s, 1H), 6.28 (d, J = 7.5 Hz, 1H), 4.40 |
| | | (s, 2H), 3.99-3.87 (m, 1H), 3.02-2.89 (m, 2H), 2.83-2.70 (m, 2H), |
| | | 1.47 (s, 3H), 1.37 (s, 6H), 1.10 (d, J = 6.4 Hz, 6H). |
| 117 | 419.3 | 1H NMR (400 MHz, DMSO) δ 7.87 (d, J = 5.4 Hz, 1H), 7.36-7.27 (m, |
| | | 4H), 6.48-6.38 (m, 2H), 4.41 (s, 2H), 3.99-3.87 (m, 1H), 2.55 (s, 1H), |
| | | 2.06 (s, 6H), 1.37 (s, 6H), 1.11 (d, J = 6.4 Hz, 6H). |
| 118 | 489.2 | ¹H NMR (400 MHz, DMSO) δ (ppm): 7.89 (d, J = 5.3 Hz, 1H), 7.58 (d, J = |
| | | 8.4 Hz, 2H), 7.50-7.40 (m, 2H), 6.70 (d, J = 6.1 Hz, 1H), 6.48 (dd, J = |
| | | 5.3, 1.3 Hz, 1H), 6.44 (s, 1H), 4.43 (s, 2H), 4.36-4.26 (m, 1H), 3.86- |
| | | 3.74 (m, 2H), 3.70-3.65 (m, 1H), 3.48-3.45 (m, 1H), 2.20-2.04 (m, 1H), |
| | | 1.78-1.66 (m, 1H), 1.41-1.32 (m, 8H), 1.16 (br s, 2H). |
| 119 | 489.3 | ¹H NMR (400 MHz, DMSO) δ (ppm): 7.90 (s, 1H), 7.58 (d, J = 8.4 Hz, |
| | | 2H), 7.50-7.40 (m, 2H), 6.70 (d, J = 5.8 Hz, 1H), 6.54-6.41 (m, 2H), |
| | | 4.42 (s, 2H), 4.31 (s, 1H), 3.88-3.75 (m, 2H), 3.70-3.65 (m, 1H), 3.47 |
| | | (dd, J = 8.7, 3.9 Hz, 1H), 2.21-1.96 (m, 1H), 1.84-1.66 (m, 1H), 1.45- |
| | | 1.32 (m, 8H), 1.16 (br s, 2H). |
| 120 | 515.3 | ¹H NMR (400 MHz, DMSO) δ (ppm): 7.87 (d, J = 5.3 Hz, 1H), 7.57 (d, J = |
| | | 8.4 Hz, 2H), 7.47-7.41 (m, 2H), 6.68 (d, J = 6.8 Hz, 1H), 6.47 (dd, J = |
| | | 5.3, 1.3 Hz, 1H), 6.33 (s, 1H), 4.59 (s, 2H), 4.47 (s, 2H), 4.41 (s, 2H), |
| | | 4.05-3.99 (m, 1H), 2.61-2.52 (m, 2H), 2.07-1.91 (m, 2H), 1.41-1.32 |
| | | (m, 8H), 1.16 (s, 2H). |
| 121 | 503.3 | ¹H NMR (400 MHz, DMSO) δ (ppm): 7.87 (d, J = 5.6 Hz, 1H), 7.58 (d, J = |
| | | 8.4 Hz, 2H), 7.48-7.41 (m, 2H), 6.45 (d, J = 3.8 Hz, 2H), 6.40 (d, J = |
| | | 7.4 Hz, 1H), 4.41 (s, 2H), 3.84-3.81 (m, 2H), 3.70-3.67 (m, 1H), 3.05 (dd, |
| | | J = 11.5, 9.3 Hz, 1H), 1.94-1.86 (m, 1H), 1.71-1.64 (m, 1H), 1.58-1.39 |
| | | (m, 3H), 1.38 (s, 6H), 1.36-1.33 (m, 2H), 1.16 (br s, 2H). |
| 122 | 475.5 | 1H NMR (500 MHz, DMSO) δ (ppm): 7.83 (d, J = 5.4 Hz, 1H), 7.59 (d, J = |
| | | 8.4 Hz, 2H), 7.48-7.45 (m, 2H), 6.54 (d, J = 3.9 Hz, 1H), 5.53 (broad |
| | | s, 1H), 4.50 (s, 2H), 4.19 (m, 1H), 2.06 (s, 3H), 1.42-1.34 (m, 8H), 1.23- |
| | | 1.15 (m, 8H). |
| 123 | 468.4 | 1H NMR (400 MHz, DMSO) δ 7.87 (d, J = 5.3 Hz, 1H), 7.68-7.62 (m, |
| | | 2H), 7.56-7.51 (m, 2H), 6.43 (dd, J = 5.3, 1.4 Hz, 1H), 6.39 (s, 1H), 6.27 |
| | | (d, J = 7.7 Hz, 1H), 4.41 (s, 2H), 4.00-3.90 (m, 1H), 3.64-3.52 (m, 2H), |
| | | 3.48-3.35 (m, 2H), 1.39 (s, 6H), 1.10 (d, J = 6.4 Hz, 6H). |
| 124 | 443.4 | 1H NMR (400 MHz, DMSO) δ (ppm): 7.87 (d, J = 5.3 Hz, 1H), 7.47 (d, J = |
| | | 8.6 Hz, 2H), 7.40 (d, J = 8.6 Hz, 2H), 6.41 (d, J = 5.3 Hz, 1H), 6.38 (s, |
| | | 1H), 6.27 (d, J = 7.6 Hz, 1H), 4.40 (s, 2H), 3.99-3.89 (m, 1H), 2.01- |
| | | 1.91 (m, 1H), 1.69-1.59 (m, 1H), 1.49 (s, 3H), 1.37 (s, 6H), 1.10 (d, J = |
| | | 6.4 Hz, 6H). |
| 125 | 420.4 | 1H NMR (400 MHz, DMSO) δ (ppm): 7.87 (d, J = 5.2 Hz, 1H), 7.67- |
| | | 7.62 (m, 2H), 7.51-7.44 (m, 2H), 6.42 (d, J = 5.3 Hz, 1H), 6.38 (s, 1H), |
| | | 6.27 (d, J = 7.5 Hz, 1H), 4.41 (s, 2H), 3.99-3.88 (m, 1H), 1.70 (s, 6H), |
| | | 1.38 (s, 6H), 1.10 (d, J = 6.4 Hz, 6H). |
| 126 | 460.4 | 1H NMR (400 MHz, DMSO) δ (ppm): 7.87 (d, J = 5.3 Hz, 1H), 7.68- |
| | | 7.62 (m, 2H), 7.51-7.45 (m, 2H), 6.42 (dd, J = 5.3, 1.3 Hz, 1H), 6.38 (s, |
| | | 1H), 6.27 (d, J = 7.4 Hz, 1H), 4.41 (s, 2H), 3.99-3.89 (m, 1H), 2.13- |
| | | 2.03 (m, 2H), 1.91-1.52 (m, 8H), 1.38 (s, 6H), 1.10 (d, J = 6.4 Hz, 6H). |
| 127 | 517.4 | 1H NMR(400 MHz, DMSO) δ (ppm): 7.89 (d, J = 4.9 Hz, 1H), 7.59 (d, J = |
| | | 8.4 Hz, 2H), 7.46 (dd, J = 8.5, 2.1 Hz, 2H), 6.64 (d, J = 6.4 Hz, 1H), |
| | | 6.47-6.44 (m, 1H), 6.41 (s, 1H), 4.43 (s, 2H), 3.86 (m, 2H), 3.67 (d, J = |
| | | 10.4 Hz, 1H), 3.41 (m, 2H), 1.93 (d, J = 12.6 Hz, 1H), 1.83 (d, J = 15.0 |
| | | Hz, 1H), 1.39 (s, 6H), 1.37 (s, 2H), 1.23 (s, 1H), 1.18 (s, 2H), 1.08 (d, J = |
| | | 6.2 Hz, 3H). |
| 128 | 517.5 | 1H NMR (400 MHz, DMSO) δ (ppm): 7.87 (t, J = 5.8 Hz, 1H), 7.60 (d, J = |
| | | 8.4 Hz, 2H), 7.47 (d, J = 8.6 Hz, 2H), 6.49-6.36 (m, 3H), 4.43 (s, 2H), |
| | | 4.17-4.05 (m, 1H), 3.81-3.73 (m, 2H), 3.60-3.44 (m, 2H), 2.04 (dd, J = |
| | | 7.2, 3.3 Hz, 1H), 1.68-1.53 (m, 1H), 1.40 (s, 6H), 1.37 (d, J = 1.6 Hz, |
| | | 2H), 1.18 (s, 2H), 0.85 (d, J = 7.1 Hz, 3H). |
| 129 | 474.4 | 1H NMR (400 MHz, DMSO) δ (ppm): 7.88 (d, J = 5.2 Hz, 1H), 7.61- |
| | | 7.55 (m, 2H), 7.52-7.46 (m, 2H), 6.48-6.38 (m, 3H), 4.42 (s, 2H), 3.93- |
| | | 3.78 (m, 3H), 3.41-3.35 (m, 2H), 2.81-2.70 (m, 2H), 2.69-2.58 (m, |
| | | 2H), 2.33-2.19 (m, 1H), 2.05-1.93 (m, 1H), 1.88-1.76 (m, 2H), 1.47- |
| | | 1.26 (m, 8H). |
| 130 | 517.4 | ¹H NMR (400 MHz, DMSO) δ (ppm): 7.87 (d, J = 5.3 Hz, 1H), 7.50- |
| | | 7.38 (m, 4H), 6.47-6.40 (m, 3H), 4.42 (s, 2H), 3.94-3.67 (m, 3H), 3.41- |
| | | 3.33 (m, 2H), 2.74-2.63 (m, 2H), 2.60 (t, J = 8.9 Hz, 2H), 2.09 (dd, J = |
| | | 17.8, 8.3 Hz, 1H), 1.99-1.86 (m, 1H), 1.83 (dd, J = 12.5, 2.4 Hz, 2H), |
| | | 1.48-1.25 (m, 8H). |
| 131 | 446.4 | 1H NMR (400 MHz, DMSO) δ (ppm): 7.87 (d, J = 5.3 Hz, 1H), 7.66- |
| | | 7.59 (m, 2H), 7.50-7.44 (m, 2H), 6.42 (dd, J = 5.3, 1.4 Hz, 1H), 6.38 (s, |
| | | 1H), 6.28 (d, J = 7.5 Hz, 1H), 4.41 (s, 2H), 3.99-3.89 (m, 1H), 2.45- |
| | | 2.38 (m, 2H), 2.15-2.03 (m, 2H), 1.94-1.85 (m, 4H), 1.38 (s, 6H), 1.10 |
| | | (d, J = 6.4 Hz, 6H). |
| 132 | 531.4 | 1H NMR (400 MHz, DMSO) δ 7.86 (d, J = 5.3 Hz, 1H), 7.60 (d, J = 8.4 |
| | | Hz, 2H), 7.47 (d, J = 8.4 Hz, 2H), 6.54 (s, 1H), 6.44 (d, J = 5.4 Hz, 1H), |
| | | 6.19 (d, J = 9.0 Hz, 1H), 4.42 (s, 2H), 4.00 (td, J = 9.8, 5.0 Hz, 1H), 3.84 |
| | | (d, J = 10.7 Hz, 1H), 3.45-3.35 (m, 2H), 3.11 (d, J = 11.2 Hz, 1H), 1.65- |
| | | 1.48 (m, 2H), 1.40 (d, J = 3.3 Hz, 6H), 1.39-1.34 (m, 2H), 1.22-1.13 |
| | | (m, 2H), 0.95 (s, 3H), 0.76 (s, 3H). |
| 133 | 551.3 | 1H NMR(400 MHz, DMSO) δ (ppm): 7.92 (d, J = 5.3 Hz, 1H), 7.60 (d, J = |
| | | 8.4 Hz, 2H), 7.46 (d, J = 8.5 Hz, 2H), 6.65 (d, J = 7.6 Hz, 1H), 6.52 (d, J = |
| | | 5.4 Hz, 1H), 6.45 (s, 1H), 4.45 (s, 2H), 4.16-4.02 (m, 1H), 3.29-3.17 |
| | | (m, 2H), 3.17-3.04 (m, 2H), 2.25-2.11 (m, 2H), 2.03-1.82 (m, 2H), |
| | | 1.40 (s, 6H), 1.37 (s, 2H), 1.18 (s, 2H) |
| 134 | 517.3 | 1H NMR (400 MHz, DMSO) δ (ppm): 7.88 (d, J = 5.2 Hz, 1H), 7.60- |
| | | 7.54 (m, 2H), 7.46-7.40 (m, 2H), 6.52 (dd, J = 5.4, 1.5 Hz, 1H), 6.50 (s, |
| | | 1H), 6.44 (d, J = 7.2 Hz, 1H), 4.64 (q, J = 7.1 Hz, 1H), 3.94-3.79 (m, |
| | | 3H), 3.43-3.35 (m, 2H), 1.90-1.81 (m, 2H), 1.68 (d, J = 7.1 Hz, 3H), |
| | | 1.53 (s, 3H), 1.44-1.33 (m, 7H), 1.19-1.12 (m, 2H). |
| 135 | 433.3 | 1H NMR (400 MHz, DMSO) δ (ppm): 7.83 (d, J = 5.4 Hz, 1H), 7.61- |
| | | 7.53 (m, 2H), 7.46-7.39 (m, 2H), 6.54 (dd, J = 5.5, 1.4 Hz, 1H), 6.49 (s, |
| | | 1H), 5.85 (s, 2H), 4.65 (q, J = 7.2 Hz, 1H), 1.68 (d, J = 7.0 Hz, 3H), 1.53 |
| | | (s, 3H), 1.39 (s, 3H), 1.39-1.34 (m, 2H), 1.19-1.14 (m, 2H). |
| 136 | 537.2 | 1H NMR (400 MHz, DMSO) δ 7.94 (d, J = 5.3 Hz, 1H), 7.58 (d, J = 8.4 |
| | | Hz, 2H), 7.51-7.38 (m, 2H), 6.94 (d, J = 6.6 Hz, 1H), 6.57 (dd, J = 5.3, |
| | | 1.3 Hz, 1H), 6.48 (s, 1H), 4.58 (dd, J = 14.3, 6.9 Hz, 1H), 4.45 (s, 2H), |
| | | 3.50 (dd, J = 13.3, 7.3 Hz, 1H), 3.30-3.25 (m, 1H), 3.20-3.07 (m, 1H), |
| | | 2.85-2.82 (m, 1H), 2.47-2.38 (m, 1H), 2.14-2.00 (m, 1H), 1.43-1.30 |
| | | (m, 8H), 1.16 (br s, 2H). |
| 137 | 525.2 | ¹H NMR (400 MHz, acetone) δ (ppm): 8.08-7.93 (m, 1H), 7.61 (d, J = |
| | | 8.5 Hz, 2H), 7.56-7.48 (m, 2H), 6.66 (d, J = 5.1 Hz, 2H), 6.06 (t, J = 5.7 |
| | | Hz, 1H), 4.55 (s, 2H), 3.84 (dd, J = 12.8, 6.4 Hz, 2H), 3.37 (t, J = 6.6 Hz, |
| | | 2H), 2.96 (s, 3H), 1.46 (s, 6H), 1.41-1.38 (m, 2H), 1.20-1.15 (m, 2H). |
| 138 | 473.3 | ¹H NMR (400 MHz, DMSO) δ (ppm): 7.94-7.86 (m, 1H), 7.58 (d, J = |
| | | 8.4 Hz, 2H), 7.47-7.38 (m, 2H), 6.75 (s, 1H), 6.52 (d, J = 4.5 Hz, 2H), |
| | | 4.47 (s, 2H), 1.39 (s, 6H), 1.37-1.34 (m, 2H), 1.28 (s, 3H), 1.16 (s, 2H), |
| | | 0.64-0.57 (m, 4H). |
| 139 | 486.3 | 1H NMR (400 MHz, DMSO) δ (ppm): 7.94 (d, J = 5.3 Hz, 1H), 7.62- |
| | | 7.56 (m, 2H), 7.49-7.44 (m, 2H), 6.72 (d, J = 6.9 Hz, 1H), 6.54 (dd, J = |
| | | 5.3, 1.3 Hz, 1H), 6.51 (s, 1H), 4.46 (s, 2H), 4.21-4.08 (m, 1H), 2.85 (dd, |
| | | J = 16.7, 5.8 Hz, 1H), 2.73 (dd, J = 16.7, 4.8 Hz, 1H), 1.41-1.35 (m, 8H), |
| | | 1.26 (d, J = 6.7 Hz, 3H), 1.20-1.15 (m, 2H). |
| 140 | 539.3 | ¹H NMR (400 MHz, DMSO) δ (ppm): 7.93 (d, J = 5.3 Hz, 1H), 7.58 (d, J = |
| | | 8.4 Hz, 2H), 7.52-7.32 (m, 2H), 6.59 (d, J = 8.0 Hz, 1H), 6.52 (dd, J = |
| | | 5.3, 1.3 Hz, 1H), 6.44 (s, 1H), 4.49-4.30 (m, 3H), 3.40 (dd, J = 14.2, 5.6 |
| | | Hz, 1H), 3.16-3.11 (m, 1H), 2.96 (s, 3H), 1.42-1.32 (m, 8H), 1.27 (d, J = |
| | | 6.6 Hz, 3H), 1.16 (br s, 2H). |
| 141 | 523.2 | 1H NMR(400 MHz, DMSO) δ (ppm): 7.98 (d, J = 5.3 Hz, 1H), 7.60 (d, J = |
| | | 8.4 Hz, 2H), 7.52-7.41 (m, 2H), 7.31 (d, J = 5.0 Hz, 1H), 6.64 (dd, J = |
| | | 5.3, 1.3 Hz, 1H), 6.55 (s, 1H), 4.62-4.51 (m, 2H), 4.49 (s, 2H), 4.47- |
| | | 4.40 (m, 1H), 4.04-3.94 (m, 2H), 1.40 (s, 6H), 1.39-1.35 (m, 2H), 1.18 |
| | | (s, 2H) |
| 142 | 477.5 | 1H NMR (400 MHz, DMSO) δ (ppm): 7.95 (d, J = 5.7 Hz, 1H), 7.59 (d, J = |
| | | 8.4 Hz, 2H), 7.46 (d, J = 8.5 Hz, 2H), 6.80 (d, J = 2.9 Hz, 1H), 6.59 (d, J = |
| | | 2.0 Hz, 2H), 4.89-4.61 (m, 1H), 4.48 (s, 2H), 2.68-2.56 (m, 1H), |
| | | 1.40 (s, 6H), 1.39-1.34 (m, 2H), 1.18 (s, 2H), 1.14-1.02 (m, 1H), 0.95- |
| | | 0.81 (m, 1H). |
| 143 | 421.4 | 1H NMR (400 MHz, DMSO) δ 8.69 (d, J = 2.3 Hz, 1H), 7.99 (dd, J = 8.5, |
| | | 2.4 Hz, 1H), 7.88 (d, J = 5.2 Hz, 1H), 7.76 (d, J = 8.5 Hz, 1H), 6.44 (d, J = |
| | | 5.3 Hz, 1H), 6.39 (s, 1H), 6.25 (d, J = 7.6 Hz, 1H), 4.43 (s, 2H), 3.95 (dq, |
| | | J = 12.8, 6.4 Hz, 1H), 1.72 (s, 6H), 1.40 (s, 6H), 1.10 (d, J = 6.4 Hz, 6H). |
| 144 | 418.4 | 1H NMR (400 MHz, DMSO) δ (ppm): 7.87 (d, J = 5.3 Hz, 1H), 7.47- |
| | | 7.41 (m, 4H), 6.42 (dd, J = 5.3, 1.4 Hz, 1H), 6.38 (s, 1H), 6.27 (d, J = 7.6 |
| | | Hz, 1H), 4.40 (s, 2H), 4.00-3.89 (m, 1H), 1.81-1.75 (m, 2H), 1.57- |
| | | 1.52 (m, 2H), 1.38 (s, 6H), 1.10 (d, J = 6.4 Hz, 6H). |
| 145 | 446.4 | 1H NMR (400 MHz, DMSO) δ (ppm): 7.87 (d, J = 5.3 Hz, 1H), 7.52- |
| | | 7.44 (m, 4H), 6.42 (dd, J = 5.3, 1.2 Hz, 1H), 6.38 (s, 1H), 6.26 (d, J = 7.6 |
| | | Hz, 1H), 4.41 (s, 2H), 4.00-3.89 (m, 1H), 1.75 (d, J = 5.7 Hz, 1H), 1.51 |
| | | (d, J = 5.7 Hz, 1H), 1.45 (s, 3H), 1.38 (s, 6H), 1.10 (d, J = 6.4 Hz, 6H), |
| | | 0.77 (s, 3H). |
| 146 | 566.3 | 1H NMR (400 MHz, DMSO) δ (ppm): 7.94 (d, J = 5.3 Hz, 1H), 7.62- |
| | | 7.56 (m, 2H), 7.49-7.43 (m, 2H), 6.78 (d, J = 6.0 Hz, 1H), 6.55 (d, J = |
| | | 5.3 Hz, 1H), 6.48 (s, 1H), 4.46 (s, 2H), 4.39-4.29 (m, 1H), 3.54 (dd, J = |
| | | 10.2. 6.0 Hz, 1H), 3.46-3.36 (m, 1H), 3.33-3.26 (m, 1H), 3.08 (dd, J = |
| | | 10.2, 4.5 Hz, 1H), 2.86 (s, 3H), 2.24-2.13 (m, 1H), 1.92-1.77 (m, 1H), |
| | | 1.39 (s, 6H), 1.39-1.35 (m, , 2H), 1.20-1.15 (m, 2H). |
| 147 | 486.3 | 1H NMR (400 MHz, DMSO) δ (ppm): 7.94 (d, J = 5.3 Hz, 1H), 7.62- |
| | | 7.56 (m, 2H), 7.50-7.43 (m, 2H), 6.72 (d, J = 6.9 Hz, 1H), 6.54 (dd, J = |
| | | 5.3, 1.3 Hz, 1H), 6.51 (s, 1H), 4.46 (s, 2H), 4.20-4.08 (m, 1H), 2.85 (dd, |
| | | J = 16.7, 5.8 Hz, 1H), 2.73 (dd, J = 16.7, 4.8 Hz, 1H), 1.40 (s, 3H), 1.40 |
| | | (s, 3H), 1.37 (dd, J = 6.9, 5.3 Hz, 2H), 1.26 (d, J = 6.7 Hz, 3H), 1.20- |
| | | 1.15 (m, 2H). |
| 148 | 501.3 | 1H NMR (400 MHz, DMSO) δ (ppm): 7.94 (d, J = 5.2 Hz, 1H), 7.62- |
| | | 7.56 (m, 2H), 7.48-7.42 (m, 2H), 6.75 (d, J = 2.3 Hz, 1H), 6.59 (dd, J = |
| | | 5.2, 1.3 Hz, 1H), 6.51 (s, 1H), 4.50 (s, 2H), 3.86 (d, J = 8.4 Hz, 2H), 3.63 |
| | | (d, J = 8.2 Hz, 2H), 2.28-2.21 (m, 1H), 1.79-1.72 (m, 2H), 1.40 (s, 6H), |
| | | 1.37 (dd, J = 6.8, 5.2 Hz, 2H), 1.20-1.15 (m, 2H). |
| 149 | 418.4 | 1H NMR (400 MHz, DMSO) δ (ppm): 7.93-7.88 (m, 1H), 7.65-7.60 |
| | | (m, 2H), 7.50-7.44 (m, 2H), 6.76-6.70 (m, 1H), 6.57-6.52 (m, 2H), |
| | | 4.47 (s, 2H), 1.70 (s, 6H), 1.39 (s, 6H), 0.69-0.61 (m, 2H), 0.40-0.34 |
| | | (m, 2H). |
| 150 | 450.4 | 1H NMR (400 MHz, DMSO) δ (ppm): 7.90-7.85 (m, 1H), 7.66-7.61 |
| | | (m, 2H), 7.51-7.45 (m, 2H), 6.46-6.41 (m, 2H), 6.30 (d, J = 7.9 Hz, |
| | | 1H), 4.41 (s, 2H), 4.13-4.01 (m, 1H), 3.36 (dd, J = 9.2, 5.1 Hz, 1H), 3.23 |
| | | (s, 3H), 3.21-3.16 (m, 1H), 1.70 (s, 6H), 1.40-1.37 (m, 6H), 1.09 (d, J = |
| | | 6.6 Hz, 3H). |
| 151 | 445.4 | 1H NMR (400 MHz, DMSO) δ 7.92 (d, J = 5.3 Hz, 1H), 7.67-7.61 (m, |
| | | 2H), 7.51-7.46 (m, 2H), 6.70 (d, J = 6.9 Hz, 1H), 6.53 (dd, J = 5.3, 1.4 |
| | | Hz, 1H), 6.50 (s, 1H), 4.44 (s, 2H), 4.19-4.07 (m, 1H), 2.83 (dd, J = |
| | | 16.7, 5.8 Hz, 1H), 2.71 (dd, J = 16.7, 4.8 Hz, 1H), 1.70 (s, 6H), 1.41- |
| | | 1.36 (m, 6H), 1.24 (d, J = 6.7 Hz, 3H). |
| 152 | 448.4 | 1H NMR (400 MHz, DMSO) δ (ppm): 7.90 (d, J = 5.3 Hz, 1H), 7.66- |
| | | 7.61 (m, 2H), 7.50-7.45 (m, 2H), 6.70 (d, J = 6.1 Hz, 1H), 6.49 (dd, J = |
| | | 5.3, 1.3 Hz, 1H), 6.45 (s, 1H), 4.43 (s, 2H), 4.35-4.27 (m, 1H), 3.85- |
| | | 3.75 (m, 2H), 3.68 (td, J = 8.1, 5.6 Hz, 1H), 3.47 (dd, J = 8.7, 3.9 Hz, 1H), |
| | | 2.17-2.04 (m, 1H), 1.78-1.67 (m, 7H), 1.38 (s, 6H). |
| 153 | 462.4 | 1H NMR (400 MHz, DMSO) δ (ppm): 7.88 (d, J = 5.3 Hz, 1H), 7.66- |
| | | 7.61 (m, 2H), 7.51-7.45 (m, 2H), 6.47-6.39 (m, 3H), 4.42 (s, 2H), 3.92- |
| | | 3.78 (m, 3H), 3.41-3.33 (m, 2H), 1.87-1.79 (m, 2H), 1.70 (s, 6H), |
| | | 1.43-1.32 (m, 8H). |
| 154 | 442.3 | 1H NMR (400 MHz, DMSO) δ (ppm): 7.95 (d, J = 5.2 Hz, 1H), 7.68- |
| | | 7.63 (m, 2H), 7.52-7.47 (m, 2H), 6.92 (t, J = 6.1 Hz, 1H), 6.60-6.55 |
| | | (m, 2H), 6.08 (tt, J = 56.6, 4.1 Hz, 1H), 4.47 (s, 2H), 3.68 (tdd, J = 15.5, |
| | | 5.9, 4.2 Hz, 2H), 1.72 (s, 6H), 1.40 (s, 6H). |
| 155 | 432.4 | 1H NMR (400 MHz, DMSO) δ (ppm): 7.91 (d, J = 5.9 Hz, 1H), 7.67- |
| | | 7.61 (m, 2H), 7.49-7.43 (m, 2H), 6.75 (s, 1H), 6.56-6.49 (m, 2H), 4.47 |
| | | (s, 2H), 1.70 (s, 6H), 1.39 (s, 6H), 1.28 (s, 3H), 0.66-0.53 (m, 4H). |
| 156 | 464.2 | ¹H NMR (400 MHz, DMSO) δ (ppm): 7.86 (d, J = 5.2 Hz, 1H), 7.67- |
| | | 7.60 (m, 2H), 7.52-7.43 (m, 2H), 6.43 (dd, J = 5.3, 1.3 Hz, 1H), 6.38 (s, |
| | | 1H), 6.27 (d, J = 8.1 Hz, 1H), 4.41 (s, 2H), 3.99-3.92 (m, 1H), 3.36-3.32 |
| | | (m, 2H), 3.17 (s, 3H), 3.01-2.97 (m, 1H), 1.70 (s, 6H), 1.63-1.59 (m, 1H), |
| | | 1.38 (d, J = 1.0 Hz, 6H), 1.07 (d, J = 6.5 Hz, 3H). |
| 157 | 505.2 | ¹H NMR (400 MHz, DMSO) δ (ppm): 7.91-7.83 (m, 1H), 7.58 (d, J = |
| | | 8.4 Hz, 2H), 7.50-7.39 (m, 2H), 6.43 (d, J = 3.9 Hz, 2H), 6.27 (d, J = 7.8 |
| | | Hz, 1H), 4.41 (s, 2H), 4.07-4.02 (m, 1H), 3.44-3.37 (m, 3H), 3.20 (dd, J = |
| | | 9.2, 6.5 Hz, 1H), 1.43-1.33 (m, 8H), 1.16 (br s, 2H), 1.11-1.03 (m, 6H). |
| 158 | 519.3 | ¹H NMR (400 MHz, DMSO) δ (ppm): 7.92-7.82 (m, 1H), 7.58 (d, J = |
| | | 8.4 Hz, 2H), 7.53-7.40 (m, 2H), 6.45-6.43 (m, 2H), 6.23 (d, J = 7.9 Hz, |
| | | 1H), 4.41 (s, 2H), 4.00 (dt, J = 13.1, 6.7 Hz, 1H), 3.56-3.45 (m, 1H), |
| | | 3.40 (dd, J = 9.2, 4.8 Hz, 1H), 3.17 (dd, J = 9.2, 6.7 Hz, 1H), 1.43-1.31 |
| | | (m, 8H), 1.16 (br s, 2H), 1.12-1.01 (m, 9H). |
| 159 | 503.3 | 1H NMR (400 MHz, DMSO) δ (ppm): 7.91 (d, J = 5.2 Hz, 1H), 7.63- |
| | | 7.57 (m, 2H), 7.48-7.42 (m, 2H), 6.81 (s, 1H), 6.55 (s, 1H), 6.53 (dd, J = |
| | | 5.2, 1.3 Hz, 1H), 4.47 (s, 2H), 3.40 (s, 2H), 3.19 (s, 3H), 1.40 (s, 6H), 1.39- |
| | | 1.34 (m, 2H), 1.21-1.5 (m, 2H), 0.79-0.74 (m, 2H), 0.67-0.62 (m, 2H). |
| 160 | 498.4 | 1H NMR(400 MHz, DMSO) δ (ppm): 7.92 (d, J = 5.8 Hz, 1H), 7.60 (d, J = |
| | | 8.4 Hz, 2H), 7.47 (d, J = 8.5 Hz, 2H), 6.93 (t, J = 6.1 Hz, 1H), 6.55 (s, |
| | | 1H), 6.54 (d, J = 1.3 Hz, 1H), 4.46 (s, 2H), 3.47 (d, J = 6.1 Hz, 2H), 1.40 |
| | | (s, 6H), 1.37 (dd, J = 6.8, 5.3 Hz, 2H), 1.21-1.13 (m, J = 5.5, 2.0 Hz, |
| | | 4H), 1.09-1.03 (m, 2H). |
| 161 | 539.4 | 1H NMR (500 MHz, DMSO) δ (ppm): 12.27 (s, 1H), 7.92 (d, J = 5.7 Hz, |
| | | 1H), 7.59 (d, J = 8.4 Hz, 2H), 7.49-7.43 (m, 2H), 7.28 (broad s, 1H), |
| | | 6.56-6.45 (m, 3H), 4.44 (s, 2H), 4.17-4.05 (m, 1H), 2.98 (dd, J = 15.4, |
| | | 4.9 Hz, 1H), 2.65-2.59 (m, 1H), 2.04-1.96 (m, 1H), 1.69-1.55 (m, |
| | | 1H), 1.40 (s, 6H), 1.37 (dd, J = 6.7, 5.2 Hz, 2H), 1.20-1.15 (m, 2H). Two |
| | | protons overlap with the DMSO peak |
| 162 | 460.2 | ¹H NMR (400 MHz, DMSO) δ (ppm): 7.93 (d, J = 5.1 Hz, 1H), 7.68- |
| | | 7.60 (m, 2H), 7.51-7.43 (m, 2H), 6.74 (d, J = 1.9 Hz, 1H), 6.57 (d, J = |
| | | 5.2 Hz, 1H), 6.50 (s, 1H), 4.48 (s, 2H), 3.84 (d, J = 8.4 Hz, 2H), 3.61 (d, J = |
| | | 8.1 Hz, 2H), 2.23 (d, J = 1.9 Hz, 1H), 1.73 (s, 2H), 1.70 (s, 6H), 1.38 (s, 6H). |
| 163 | 491.4 | 1H NMR (400 MHz, DMSO) δ (ppm): 7.89 (d, J = 5.3 Hz, 1H), 7.63- |
| | | 7.56 (m, 2H), 7.50-7.43 (m, 2H), 6.51 (s, 1H), 6.50-6.44 (m, 2H), 4.43 |
| | | (s, 2H), 3.50-3.41 (m, 1H), 3.30-3.25 (m, 2H), 3.25 (s, 3H), 1.39 (s, |
| | | 6H), 1.38-1.35 (m, 2H), 1.22-1.13 (m, 2H), 1.08 (d, J = 6.2 Hz, 3H). |
| 164 | 566.4 | 1H NMR (400 MHz, DMSO-D6) δ (ppm): 7.94 (br d, J = 2.6 Hz, 1H), |
| | | 7.32 (d, J = 1.8 Hz, 1H), 7.15 (d, J = 8.2 Hz, 1H), 7.06 (dd, J = 8.1, 1.8 |
| | | Hz, 1H), 6.75 (s, 1H), 6.59 (d, J = 4.8 Hz, 1H), 6.50 (s, 1H), 4.49 (s, 2H), |
| | | 3.98 (s, 2H), 3.85 (dd, J = 8.4, 0.7 Hz, 2H), 3.62 (d, J = 7.7 Hz, 2H), 3.04 |
| | | (s, 3H), 2.24 (d, J = 1.5 Hz, 1H), 2.08 (residual acetone), 1.74 (app t, J = |
| | | 2.4 Hz, 2H), 1.39 (s, 6H), 1.23 (s, 3H), 1.17 (d, J = 5.0 Hz, 1H), 1.14 (s, |
| | | 3H), 0.94 (d, J = 5.1 Hz, 1H). |
| 165 | 524.3 | 1H NMR (400 MHz, DMSO) δ (ppm): 8.56 (d, J = 2.1 Hz, 1H), 8.38 (dd, |
| | | J = 4.7, 1.6 Hz, 1H), 7.84 (d, J = 5.2 Hz, 1H), 7.72 (dt, J = 7.9, 1.9 Hz, |
| | | 1H), 7.64-7.56 (m, 2H), 7.50-7.44 (m, 2H), 7.29 (ddd, J = 7.9, 4.8, 0.5 |
| | | Hz, 1H), 7.05 (d, J = 7.6 Hz, 1H), 6.49-6.44 (m, 2H), 5.02 (p, J = 6.8 Hz, |
| | | 1H), 4.42 (s, 2H), 1.43 (d, J = 7.0 Hz, 3H), 1.38-1.35 (m, 5H), 1.31 (s, |
| | | 3H), 1.21-1.13 (m, 2H). |
| 166 | 497.2 | 1H NMR (400 MHz, DMSO) δ (ppm): 8.15 (br. s, 1H), 7.63-7.57 (m, |
| | | 2H), 7.50-7.44 (m, 2H), 7.06-6.93 (m, 2H), 4.60 (s, 2H), 3.25 (br. s, |
| | | 3H), 1.41 (s, 6H), 1.39-1.33 (m, 2H), 1.21-1.13 (m, 2H). |
| 167 | 527.4 | 1H NMR (400 MHz, DMSO) δ 7.90 (d, J = 5.2 Hz, 1H), 7.62-7.57 (m, |
| | | 2H), 7.52 (s, 1H), 7.48-7.44 (m, 2H), 7.31 (s, 1H), 6.58 (d, J = 8.0 Hz, |
| | | 1H), 6.47 (dd, J = 5.3, 1.3 Hz, 1H), 6.44 (s, 1H), 5.01 (p, J = 6.7 Hz, 1H), |
| | | 4.43 (s, 2H), 3.74 (s, 3H), 1.40-1.35 (m, 11H), 1.21-1.15 (m, 2H). |
| 168 | 525.4 | 1H NMR (400 MHz, DMSO) δ 8.61 (s, 1H), 8.56-8.51 (m, 1H), 8.46 (d, |
| | | J = 2.5 Hz, 1H), 7.83 (d, J = 5.3 Hz, 1H), 7.63-7.57 (m, 2H), 7.50-7.45 |
| | | (m, 2H), 7.08 (d, J = 7.3 Hz, 1H), 6.55 (s, 1H), 6.49 (d, J = 5.2 Hz, 1H), |
| | | 5.12 (p, J = 6.9 Hz, 1H), 4.44 (s, 2H), 1.47 (d, J = 7.0 Hz, 3H), 1.41-1.32 |
| | | (m, 8H), 1.21-1.14 (m, 2H). |
| 169 | 513.3 | 1H NMR (400 MHz, DMSO) δ 7.93 (d, J = 5.3 Hz, 1H), 7.62-7.57 (m, |
| | | 2H), 7.56 (s, 1H), 7.48-7.44 (m, 2H), 7.33 (s, 1H), 6.66 (t, J = 5.6 Hz, |
| | | 1H), 6.50 (dd, J = 5.3, 1.3 Hz, 1H), 6.46 (s, 1H), 4.44 (s, 2H), 4.24 (d, J = |
| | | 5.5 Hz, 2H), 3.75 (s, 3H), 1.42-1.33 (m, 8H), 1.21-1.13 (m, 2H). |
| 170 | 486.3 | 1H NMR (400 MHz, DMSO) δ 7.92 (d, J = 5.1 Hz, 1H), 7.52-7.48 (m, |
| | | 2H), 7.48-7.44 (m, 2H), 6.70 (d, J = 2.3 Hz, 1H), 6.57 (dd, J = 5.2, 1.4 |
| | | Hz, 1H), 6.50 (s, 1H), 4.48 (s, 2H), 3.84 (d, J = 8.4 Hz, 2H), 3.61 (d, J = |
| | | 8.2 Hz, 2H), 2.26-2.21 (m, 1H), 1.77-1.70 (m, 3H), 1.50 (d, J = 5.7 Hz, |
| | | 1H), 1.45 (s, 3H), 1.38 (s, 6H), 0.77 (s, 3H). |
| 171 | 499.3 | 1H NMR (400 MHz, DMSO) δ 12.57 (br. s, 1H), 7.91 (d, J = 5.2 Hz, 1H), |
| | | 7.63-7.53 (m, 3H), 7.48-7.37 (m, 3H), 6.61 (t, J = 5.5 Hz, 1H), 6.49- |
| | | 6.46 (m, 1H), 6.46 (s, 1H), 4.42 (s, 2H), 4.26 (d, J = 5.4 Hz, 2H), 1.40- |
| | | 1.31 (m, 8H), 1.19-1.12 (m, 2H). |
| 172 | 602.2 | 1H NMR (400 MHz, DMSO) δ 7.88 (d, J = 5.3 Hz, 1H), 7.67 (d, J = 8.3 |
| | | Hz, 2H), 7.58 (d, J = 8.4 Hz, 2H), 7.48 (d, J = 8.3 Hz, 2H), 7.46-7.41 (m, |
| | | 2H), 7.36-7.32 (m, 1H), 7.15-7.12 (m, 1H), 6.54-6.47 (m, 2H), 4.53 (d, J = |
| | | 6.0 Hz, 2H), 4.43 (s, 2H), 2.36 (d, J = 5.0 Hz, 3H), 1.41-1.31 (m, 8H), |
| | | 1.16 (br s, 2H). |
| 173 | 499.3 | 1H NMR (400 MHz, DMSO) δ 11.77 (br. s, 1H), 7.94 (d, J = 5.2 Hz, 1H), |
| | | 7.62-7.57 (m, 2H), 7.51-7.45 (m, 2H), 6.98 (s, 1H), 6.84 (t, J = 5.5 Hz, |
| | | 1H), 6.78 (s, 1H), 6.56 (s, 1H), 6.53 (dd, J = 5.3, 1.4 Hz, 1H), 4.45 (s, |
| | | 2H), 4.42 (d, J = 5.5 Hz, 2H), 1.41-1.34 (m, 8H), 1.21-1.15 (m, 2H). |
| 174 | 500.3 | 1H NMR (400 MHz, DMSO) δ 8.12 (br. s, 1H), 7.91 (d, J = 5.3 Hz, 1H), |
| | | 7.63-7.56 (m, 2H), 7.50-7.44 (m, 2H), 6.93 (br. s, 1H), 6.56 (s, 1H), |
| | | 6.53 (d, J = 5.3 Hz, 1H), 4.51 (d, J = 5.7 Hz, 2H), 4.45 (s, 2H), 1.41-1.35 |
| | | (m, 8H), 1.21-1.15 (m, 2H). |
| 175 | 550.4 | 1H NMR (400 MHz, DMSO) δ 8.84 (dt, J = 6.8, 1.0 Hz, 1H), 7.90 (d, J = |
| | | 5.3 Hz, 1H), 7.72 (dt, J = 9.0, 1.1 Hz, 1H), 7.64-7.57 (m, 3H), 7.51- |
| | | 7.44 (m, 2H), 7.13 (td, J = 6.9, 1.3 Hz, 1H), 7.08 (t, J = 5.9 Hz, 1H), 6.60 |
| | | (s, 1H), 6.52 (dd, J = 5.3, 1.3 Hz, 1H), 4.70 (d, J = 5.9 Hz, 2H), 4.46 (s, |
| | | 2H), 1.43-1.32 (m, 8H), 1.23-1.14 (m, 2H). |
| 176 | 513.3 | 1H NMR (400 MHz, DMSO) δ 7.95 (d, J = 5.2 Hz, 1H), 7.63-7.57 (m, |
| | | 2H), 7.52 (d, J = 0.6 Hz, 1H), 7.48-7.44 (m, 2H), 6.79 (d, J = 0.9 Hz, |
| | | 1H), 6.76 (t, J = 5.4 Hz, 1H), 6.53 (dd, J = 5.3, 1.4 Hz, 1H), 6.51 (s, 1H), |
| | | 4.45 (s, 2H), 4.41 (d, J = 5.3 Hz, 2H), 3.58 (s, 3H), 1.42-1.33 (m, 8H), |
| | | 1.20-1.15 (m, 2H). |
| 177 | 511.3 | 1H NMR (400 MHz, DMSO) δ 8.73 (d, J = 4.9 Hz, 2H), 7.85 (d, J = 5.3 |
| | | Hz, 1H), 7.64-7.57 (m, 2H), 7.52-7.45 (m, 2H), 7.36 (t, J = 4.9 Hz, |
| | | 1H), 7.02 (t, J = 5.9 Hz, 1H), 6.61 (s, 1H), 6.49 (dd, J = 5.3, 1.3 Hz, 1H), |
| | | 4.66 (d, J = 5.9 Hz, 2H), 4.45 (s, 2H), 1.42-1.33 (m, 8H), 1.21-1.15 (m, 2H). |
| 178 | 479.2 | 1H NMR (400 MHz, DMSO) δ (ppm): 8.14-8.04 (m, 2H), 7.89 (d, J = |
| | | 5.3 Hz, 1H), 7.76 (d, J = 8.9 Hz, 2H), 6.46 (dd, J = 5.3, 1.2 Hz, 1H), 6.41 |
| | | (s, 1H), 6.25 (d, J = 7.6 Hz, 1H), 4.44 (s, 2H), 4.05-3.87 (m, 1H), 1.42 |
| | | (s, 6H), 1.12 (d, J = 6.4 Hz, 6H). |
| 179 | 437.3 | 1H NMR (400 MHz, DMSO) δ (ppm): 8.11-8.02 (m, 2H), 7.82 (d, J = |
| | | 5.2 Hz, 1H), 7.74 (d, J = 8.8 Hz, 2H), 6.50 (dd, J = 5.3, 1.2 Hz, 1H), 6.42 |
| | | (s, 1H), 5.85 (s, 2H), 4.43 (s, 2H), 1.39 (s, 6H). |
| 180 | 486.5 | 1H NMR (500 MHz, DMSO) δ 8.36 (d, J = 2.0 Hz, 1H), 8.21 (d, J = 8.6 |
| | | Hz, 1H), 8.15-8.09 (m, 1H), 7.95 (d, J = 5.2 Hz, 1H), 6.71 (d, J = 2.3 Hz, |
| | | 1H), 6.64 (dd, J = 5.2, 1.4 Hz, 1H), 6.55 (s, 1H), 4.52 (s, 2H), 3.88 (d, J = |
| | | 8.4 Hz, 2H), 3.64 (d, J = 8.2 Hz, 2H), 2.31-2.26 (m, 1H), 1.79-1.73 (m, |
| | | 2H), 1.44 (s, 6H). |
| 181 | 511.3 | 1H NMR (400 MHz, DMSO) δ 9.07 (d, J = 1.3 Hz, 1H), 8.67 (d, J = 5.2 |
| | | Hz, 1H), 7.87 (d, J = 5.3 Hz, 1H), 7.63-7.56 (m, 2H), 7.50-7.43 (m, |
| | | 2H), 7.35 (dd, J = 5.2, 1.3 Hz, 1H), 7.23 (t, J = 6.1 Hz, 1H), 6.59 (s, 1H), |
| | | 6.55 (dd, J = 5.3, 1.3 Hz, 1H), 4.54 (d, J = 6.0 Hz, 2H), 4.47 (s, 2H), 1.44- |
| | | 1.34 (m, 8H), 1.22-1.15 (m, 2H). |
| 182 | 511.3 | 1H NMR (400 MHz, DMSO) δ 8.57-8.56 (m, 1H), 8.56-8.54 (m, 1H), |
| | | 8.49 (d, J = 2.5 Hz, 1H), 7.89 (d, J = 5.3 Hz, 1H), 7.62-7.57 (m, 2H), |
| | | 7.50-7.43 (m, 2H), 7.20 (t, J = 6.0 Hz, 1H), 6.58 (s, 1H), 6.54 (dd, J = |
| | | 5.3, 1.0 Hz, 1H), 4.60 (d, J = 6.0 Hz, 2H), 4.46 (s, 2H), 1.40-1.36 (m, |
| | | 8H), 1.22-1.14 (m, 2H). |
| 183 | 513.3 | 1H NMR (400 MHz, DMSO) δ 7.94 (d, J = 5.3 Hz, 1H), 7.64-7.56 (m, |
| | | 2H), 7.53-7.46 (m, 2H), 7.05 (s, 1H), 6.86 (t, J = 5.3 Hz, 1H), 6.74 (s, |
| | | 1H), 6.62 (s, 1H), 6.52 (d, J = 5.3 Hz, 1H), 4.48-4.42 (m, 4H), 3.62 (s, |
| | | 3H), 1.40-1.34 (m, 8H), 1.21-1.15 (m, 2H). |
| 184 | 499.3 | 1H NMR (400 MHz, DMSO) δ 12.57 (s, 1H), 7.93 (d, J = 5.3 Hz, 1H), |
| | | 7.62-7.57 (m, 2H), 7.56-7.49 (m, 1H), 7.49-7.44 (m, 2H), 6.86-6.65 |
| | | (m, 1H), 6.62-6.39 (m, 2H), 6.13 (d, J = 1.9 Hz, 1H), 4.45 (s, 2H), 4.42 |
| | | (d, J = 5.6 Hz, 2H), 1.42-1.33 (m, 9H), 1.21-1.14 (m, 2H). |
| 185 | 448.4 | 1H NMR (500 MHz, DMSO) δ 7.91 (d, J = 5.2 Hz, 1H), 7.14 (d, J = 8.3 |
| | | Hz, 2H), 7.04 (dd, J = 7.8, 1.5 Hz, 1H), 6.46 (d, J = 5.3 Hz, 1H), 6.42 (s, |
| | | 1H), 6.29 (d, J = 7.6 Hz, 1H), 4.43 (s, 2H), 3.98 (dd, J = 13.4, 6.6 Hz, 1H), |
| | | 3.23 (s, 3H), 1.68 (t, J = 3.6 Hz, 2H), 1.57 (t, J = 3.6 Hz, 2H), 1.42 (s, |
| | | 6H), 1.13 (d, J = 6.4 Hz, 6H). |
| 186 | 450.4 | 1H NMR (500 MHz, DMSO) δ 7.90 (d, J = 5.3 Hz, 1H), 7.84 (d, J = 8.1 |
| | | Hz, 1H), 7.76 (d, J = 1.7 Hz, 1H), 7.67 (dd, J = 8.1, 1.9 Hz, 1H), 6.47 (d, J = |
| | | 5.3 Hz, 1H), 6.42 (s, 1H), 6.26 (d, J = 7.6 Hz, 1H), 4.44 (s, 2H), 4.02- |
| | | 3.95 (m, 1H), 2.96 (s, 3H), 1.47 (s, 6H), 1.42 (s, 6H), 1.13 (d, J = 6.4 Hz, 6H). |
| 187 | 434.4 | 1H NMR (400 MHz, DMSO) δ 10.67 (s, 1H), 7.84 (d, J = 5.1 Hz, 1H), |
| | | 7.02 (d, J = 8.3 Hz, 1H), 6.96-6.91 (m, 1H), 6.45-6.34 (m, 2H), 6.26 (s, |
| | | 1H), 4.37 (s, 2H), 3.92 (dd, J = 13.0, 6.7 Hz, 1H), 2.52-2.48 (m, 1H), |
| | | 1.56 (s, 2H), 1.46 (s, 2H), 1.35 (s, 6H), 1.08 (d, J = 6.4 Hz, 6H). |
| 188 | 436.4 | 1H NMR (400 MHz, DMSO-d6) δ (ppm): 7.89 (d, J = 5.2 Hz, 1H), 7.08 |
| | | (d, J = 7.7 Hz, 1H), 6.59 (dd, J = 7.7, 1.8 Hz, 1H), 6.46 (d, J = 1.7 Hz, |
| | | 1H), 6.42 (dd, J = 5.3, 1.4 Hz, 1H), 6.38 (s, 1H), 6.30 (d, J = 7.6 Hz, 1H), |
| | | 4.40 (s, 2H), 3.96 (dq, J = 12.9, 6.4 Hz, 1H), 3.10 (s, 2H), 2.71 (s, 3H), |
| | | 1.37 (s, 6H), 1.26 (s, 6H), 1.12 (d, J = 6.4 Hz, 6H). |
| 189 | 423.4 | 1H NMR (400 MHz, DMSO-d6) δ (ppm): 7.88 (d, J = 5.2 Hz, 1H), 7.30 |
| | | (d, J = 7.9 Hz, 1H), 6.89 (dd, J = 7.9, 1.8 Hz, 1H), 6.82 (d, J = 1.7 Hz, |
| | | 1H), 6.42 (dd, J = 5.3, 1.4 Hz, 1H), 6.38 (s, 1H), 6.29 (d, J = 7.5 Hz, 1H), |
| | | 4.40 (s, 2H)- 4.28 (s, 2H), 4.01-3.92 (m, 1H), 1.37 (s, 6H), 1.32 (s, 6H), |
| | | 1.12 (d, J = 6.4 Hz, 6H). |
| 190 | 471.2 | 1H NMR (500 MHz, DMSO) δ 7.90 (d, J = 5.3 Hz, 1H), 7.85 (d, J = 8.5 |
| | | Hz, 2H), 7.79 (dd, J = 8.3, 1.8 Hz, 1H), 6.48 (d, J = 5.3 Hz, 1H), 6.43 (s, |
| | | 1H), 6.23 (d, J = 7.5 Hz, 1H), 4.44 (s, 2H), 4.02-3.93 (m, 1H), 3.59 (s, |
| | | 2H), 1.51 (s, 6H), 1.42 (s, 7H), 1.13 (d, J = 6.4 Hz, 6H). |
| 191 | 448.4 | 1H NMR (500 MHz, DMSO) δ (ppm): 7.90 (d, J = 5.3 Hz, 1H), 7.34 (d, J = |
| | | 7.7 Hz, 1H), 6.64 (dd, J = 7.7, 1.8 Hz, 1H), 6.46 (t, J = 3.3 Hz, 1H), 6.43 |
| | | (dd, J = 5.3, 1.4 Hz, 1H), 6.40 (s, 1H), 6.29 (d, J = 7.6 Hz, 1H), 4.42 (d, J = |
| | | 14.4 Hz, 2H), 3.97 (dq, J = 12.9, 6.4 Hz, 1H), 3.41 (s, 2H), 2.71 (s, 3H), |
| | | 2.26 (dt, J = 11.6, 8.8 Hz, 2H), 2.18 (ddd, J = 11.6, 8.6, 5.7 Hz, 2H), 2.05- |
| | | 1.94 (m, 2H), 1.40 (d, J = 18.3 Hz, 6H), 1.13 (dd, J = 6.4, 3.3 Hz, 6H). |
| 192 | 511.3 | 1H NMR (400 MHz, DMSO) δ 8.04 (d, J = 8.5 Hz, 1H), 7.98 (t, J(H—F) = |
| | | 56 Hz, 1H), 7.90 (d, J = 5.2 Hz, 1H), 7.80 (d, J = 2.2 Hz, 1H), 7.62 (dd, J = |
| | | 8.5, 2.2 Hz, 1H), 6.47 (dd, J = 5.3, 1.4 Hz, 1H), 6.42 (s, 1H), 6.30 (d, J = |
| | | 7.6 Hz, 1H), 4.44 (s, 2H), 4.00-3.93 (m, 1H), 1.42 (s, 6H), 1.12 (d, J = |
| | | 6.4 Hz, 6H). |
| 193 | 511.3 | 1H NMR (400 MHz, DMSO) δ 8.04 (d, J = 8.5 Hz, 1H), 7.98 (t, J(H—F) = |
| | | 56 Hz, 1H), 7.90 (d, J = 5.2 Hz, 1H), 7.80 (d, J = 2.2 Hz, 1H), 7.62 (dd, J = |
| | | 8.5, 2.2 Hz, 1H), 6.47 (dd, J = 5.3, 1.4 Hz, 1H), 6.42 (s, 1H), 6.30 (d, J = |
| | | 7.6 Hz, 1H), 4.44 (s, 2H), 4.00-3.93 (m, 1H), 1.42 (s, 6H), 1.12 (d, J = |
| | | 6.4 Hz, 6H). |
| 194 | 500.4 | 1H NMR (400 MHz, DMSO) δ (ppm): 7.85 (d, J = 5.2 Hz, 1H), 7.35 (d, J = |
| | | 6.2 Hz, 1H), 7.24 (s, 1H), 7.06 (d, J = 7.9 Hz, 1H), 6.38 (s, 1H), 6.35 (s, |
| | | 1H), 6.26 (d, J = 6.9 Hz, 1H), 4.37 (s, 2H), 3.99-3.85 (m, 1H), 3.68 (s, |
| | | 2H), 3.03 (d, J = 1.8 Hz, 3H), 1.35 (s, 6H), 1.29 (s, 6H), 1.07 (d, J = 6.3, 6H). |
| 195 | 512.4 | 1H NMR (500 MHz, DMSO) δ (ppm): 7.90 (d, J = 5.3 Hz, 1H), 7.67 (d, J = |
| | | 8.0 Hz, 1H), 7.28 (d, J = 1.8 Hz, 1H), 7.17 (dd, J = 8.0, 1.8 Hz, 1H), |
| | | 6.45 (d, J = 5.3 Hz, 1H), 6.41 (s, 1H), 6.29 (d, J = 6.5 Hz, 1H), 4.43 (s, |
| | | 2H), 4.08 (s, 2H), 3.97 (dd, J = 13.9, 6.4 Hz, 1H), 3.02 (s, 3H), 2.34 (dd, J = |
| | | 11.5, 5.5 Hz, 4H), 2.04 (dd, J = 15.3, 7.9 Hz, 2H), 1.41 (s, 6H), 1.13 (d, |
| | | J = 6.4 Hz, 6H). |
| 196 | 518.4 | 1H NMR (500 MHz, DMSO) δ (ppm): 8.00 (s, 1H), 7.91 (d, J = 5.3 Hz, |
| | | 1H), 7.37 (d, J = 8.0 Hz, 1H), 7.04 (dd, J = 8.0, 1.8 Hz, 1H), 6.72 (d, J = |
| | | 6.1 Hz, 1H), 6.49 (d, J = 5.3 Hz, 1H), 6.45 (s, 1H), 4.43 (s, 2H), 4.37- |
| | | 4.29 (m, 1H), 4.12 (s, 2H), 3.88-3.77 (m, 2H), 3.70 (ddd, J = 8.1, 5.7 Hz, |
| | | 1H), 3.49 (dd, J = 8.7, 3.9 Hz, 1H), 2.18-2.09 (m, 1H), 2.01-1.92 (m, |
| | | 1H), 1.81-1.71 (m, 1H), 1.39 (s, 6H), 1.36 (s, 6H), 0.91-0.82 (m, 4H). |
| 197 | 437.4 | 1H NMR (400 MHz, DMSO-D6) δ 7.89 (d, J = 5.3 Hz, 1H), 7.70 (d, J = |
| | | 1.9 Hz, 1H), 6.83 (d, J = 1.9 Hz, 1H), 6.44 (dd, J = 5.3, 1.3 Hz, 1H), 6.40 |
| | | (s, 1H), 6.32 (d, J = 7.4 Hz, 1H)- 4.42 (s, 2H), 4.01-3.92 (m, 1H), 3.22 |
| | | (s, 2H), 2.76 (s, 3H), 1.39 (s, 6H), 1.27 (s, 6H), 1.12 (d, J = 6.4 Hz, 6H). |
| 198 | 453.4 | 1H NMR (400 MHz, DMSO-D6) δ (ppm): 7.91-7.87 (m, 1H), 7.30 (d, J = |
| | | 7.9 Hz, 1H), 6.90 (dd, J = 7.9, 1.8 Hz, 1H), 6.83 (d, J = 1.7 Hz, 1H), |
| | | 6.47-6.41 (m, 2H), 6.32 (d, J = 7.9 Hz, 1H), 4.41 (s, 2H), 4.28 (s, 2H), |
| | | 4.15-4.04 (m, 1H), 3.38 (dd, J = 9.2, 5.1 Hz, 1H), 3.25 (s, 3H), 3.21 (dd, |
| | | J = 9.2, 6.2 Hz, 1H), 1.37 (app d, J = 2.3 Hz, 6H), 1.32 (s, 6H), 1.10 (d, J = |
| | | 6.6 Hz, 6H). |
| 199 | 465.4 | 1H NMR (400 MHz, DMSO-D6) δ (ppm): 7.91 (br s, 1H), 7.30 (d, J = 7.9 |
| | | Hz, 1H), 6.90 (d, J = 1.8 Hz, 1H), 6.82 (d, J = 1.7 Hz, 1H), 6.49 (br s, 2H), |
| | | 4.43 (s, 2H), 4.28 (s, 2H), 3.89-3.82 (m, 3H), 3.42-3.35 (m, 2H), 1.89- |
| | | 1.82 (m, 2H), 1.45-1.39 (m, 2H), 1.38 (s, 6H), 1.32 (s, 6H). One acidic |
| | | proton is missing. |
| 200 | 498.3 | 1H NMR (500 MHz, DMSO) δ (ppm): 7.89 (d, J = 5.3 Hz, 1H), 7.30 (d, J = |
| | | 1.7 Hz, 1H), 7.06 (dd, J = 8.0, 1.8 Hz, 1H), 6.96 (d, J = 8.1 Hz, 1H), |
| | | 6.44 (dd, J = 5.3, 1.3 Hz, 1H), 6.40 (s, 1H), 6.28 (d, J = 7.6 Hz, 1H), 4.42 |
| | | (s, 2H), 4.01 (s, 2H), 3.96 (dt, J = 13.0, 6.5 Hz, 1H), 3.07 (s, 3H), 1.40 (s, |
| | | 6H), 1.19 (dd, J = 6.9, 4.5 Hz, 2H), 1.12 (d, J = 6.4 Hz, 6H), 1.09 (dd, J = |
| | | 6.8, 4.4 Hz, 2H). |
| 201 | 528.5 | 1H NMR (500 MHz, DMSO) δ (ppm): 7.92 (d, J = 5.5 Hz, 1H), 7.40 (d, J = |
| | | 8.0 Hz, 1H), 7.30 (d, J = 1.7 Hz, 1H), 7.11 (dd, J = 8.0, 1.8 Hz, 1H), |
| | | 6.70 (d, J = 6.2 Hz, 1H), 6.51 (dd, J = 5.3, 1.4 Hz, 1H), 6.47 (s, 1H), 4.44 |
| | | (s, 2H), 4.39-4.30 (m, 1H), 3.89-3.78 (m, 2H), 3.74 (s, 2H), 3.70 |
| | | (dddd, J = 8.1, 5.7 Hz, 1H), 3.49 (dd, J = 8.7, 3.9 Hz, 1H), 2.19-2.10 (m, |
| | | 1H), 1.81-1.72 (m, 1H), 1.40 (s, 6H), 1.35 (s, 6H). |
| 202 | 466.5 | 1H NMR (500 MHz, DMSO) δ (ppm): 7.90 (d, J = 5.9 Hz, 1H), 7.08 (d, J = |
| | | 7.7 Hz, 1H), 6.60 (dd, J = 7.7, 1.8 Hz, 1H), 6.46 (dd, J = 10.9, 3.0 Hz, |
| | | 3H), 6.32 (d, J = 7.8 Hz, 1H), 4.41 (s, 2H), 4.15-4.06 (m, 1H), 3.39 (dd, |
| | | J = 9.2, 5.1 Hz, 1H), 3.26 (s, 3H), 3.22 (dd, J = 9.2, 6.2 Hz, 1H), 3.11 (s, |
| | | 2H), 2.72 (s, 3H), 1.41-1.35 (m, 6H), 1.27 (s, 6H), 1.12 (d, J = 6.6 Hz, 3H). |
| 203 | 530.4 | ¹H NMR (500 MHz, DMSO) δ (ppm): 7.91-7.86 (m, 1H), 7.40 (d, J = |
| | | 8.0 Hz, 1H), 7.30 (d, J = 1.7 Hz, 1H), 7.11 (dd, J = 8.0, 1.8 Hz, 1H), 6.48- |
| | | 6.43 (m, 2H), 6.29 (d, J = 7.8 Hz, 1H), 4.43 (s, 2H), 4.15-4.04 (m, |
| | | 1H), 3.74 (s, 2H), 3.38 (dd, J = 9.2, 5.1 Hz, 1H), 3.25 (s, 3H), 3.22 (dd, J = |
| | | 9.2, 6.2 Hz, 1H), 3.07 (s, 3H), 1.43-1.38 (m, 6H), 1.35 (s, 6H), 1.11 (d, |
| | | J = 6.6 Hz, 3H). |
| 204 | 540.4 | ¹H NMR (500 MHz, DMSO) δ (ppm): 7.92 (d, J = 5.2 Hz, 1H), 7.67 (d, J = |
| | | 8.0 Hz, 1H), 7.28 (d, J = 1.7 Hz, 1H), 7.17 (dd, J = 8.0, 1.8 Hz, 1H), |
| | | 6.70 (d, J = 6.2 Hz, 1H), 6.51 (dd, J = 5.3, 1.4 Hz, 1H), 6.47 (s, 1H), 4.44 |
| | | (s, 2H), 4.39-4.31 (m, 1H)- 4.08 (s, 2H), 3.87-3.81 (m, 2H), 3.71 (td, J = |
| | | 8.1, 5.7 Hz, 1H), 3.49 (dd, J = 8.8, 3.9 Hz, 1H), 3.02 (s, 3H), 2.34 (dd, J = |
| | | 11.—, 5.5 Hz, 4H), 2.17-2.13 (m, 1H), 2.07-1.98 (m, 2H), 1.82-1.72 |
| | | (m, 1H), 1.41 (s, 6H). |
| 205 | 542.4 | ¹H NMR (500 MHz, DMSO) δ (ppm): 7.90 (d, J = 5.9 Hz, 1H), 7.67 (d, J = |
| | | 8.0 Hz, 1H), 7.29 (d, J = 1.7 Hz, 1H), 7.18 (dd, J = 8.0, 1.8 Hz, 1H), |
| | | 6.46 (d, J = 4.1 Hz, 2H), 6.30 (d, J = 7.9 Hz, 1H), 4.43 (s, 2H), 4.11 (dd, J = |
| | | 13.0, 6.5 Hz, 1H), 4.08 (s, 2H), 3.39 (dd, J = 9.2, 5.1 Hz, 1H), 3.26 (s, |
| | | 3H), 3.22 (dd, J = 9.2, 6.2 Hz, 1H), 3.02 (s, 3H), 2.34 (dd, J = 11.4, 5.6 |
| | | Hz, 4H), 2.08-1.99 (m, 2H), 1.41 (d, J = 2.6 Hz, 6H), 1.11 (d, J = 6.6 Hz, 3H). |
| 206 | 464.5 | ¹H NMR (500 MHz, DMSO) δ (ppm): 7.92 (d, J = 5.3 Hz, 1H), 7.08 (d, J = |
| | | 7.7 Hz, 1H), 6.73 (s, 1H), 6.60 (dd, J = 7.7, 1.8 Hz, 1H), 6.50 (d, J = 5.3 |
| | | Hz, 1H), 6.47 (d, J = 1.7 Hz, 2H), 4.43 (s, 2H), 4.38-4.31 (m, 1H), 3.88- |
| | | 3.79 (m, 2H), 3.71 (ddd, J = 8.1, 5.7 Hz, 1H), 3.50 (dd, J = 8.8, 3.9 Hz, |
| | | 1H), 3.11 (s, 2H), 2.72 (s, 3H), 2.20-2.11 (m, 1H), 1.82-1.73 (m, 1H), |
| | | 1.38 (s, 6H), 1.27 (s, 6H). |
| 207 | 540.3 | ¹H NMR (400 MHz, DMSO-D6) δ (ppm): 7.94 (d, J = 5.2 Hz, 1H), 7.39 |
| | | (d, J = 8.0 Hz, 1H), 7.28 (d, J = 1.8 Hz, 1H), 7.09 (dd, J = 8.0, 1.8 Hz, |
| | | 1H), 6.75 (d, J = 2.2 Hz, 1H), 6.58 (dd, J = 5.2, 1.2 Hz, 1H), 6.50 (s, 1H), |
| | | 4.49 (s, 2H), 3.85 (d, J = 8.4 Hz, 2H), 3.73 (s, 2H), 3.63 (d, J- = 8.2 Hz, |
| | | 2H), 3.17-3.11 (m, 1H), 3.06 (s, 3H), 2.24 (dd, J = 4.6, 2.5 Hz, 1H), 1.75 |
| | | (overlapped s, 1H), 1.39 (s, 6H), 1.34 (s, 6H). |
| 208 | 516.3 | ¹H NMR (400 MHz, DMSO-D6) δ (ppm): 7.98-7.92 (m, 1H), 7.39 (d, J = |
| | | 8.0 Hz, 1H), 7.28 (d, J = 1.7 Hz, 1H), 7.10 (dd, J = 8.0, 1.8 Hz, 1H), |
| | | 6.81 (d, J = 3.0 Hz, 1H), 6.61-6.56 (m, 2H), 4.75 (dddd, J = 65.1, 6.2, |
| | | 4.9, 3.1 Hz, 1H), 4.48 (s, 2H), 3.73 (s, 2H), 3.07 (s, 3H), 2.65-2.56 (m, |
| | | 1H), 1.39 (app d, J = 2.5 Hz, 6H), 1.34 (s, 6H), 1.15-1.03 (m, 1H), 0.88 |
| | | (dddd, J = 24.8, 7.6, 5.7, 3.0 Hz, 1H). |
| 209 | 542.5 | ¹H NMR (500 MHz, DMSO) δ (ppm): 7.90 (d, J = 5.3 Hz, 1H), 7.40 (d, J = |
| | | 8.0 Hz, 1H), 7.30 (d, J = 1.8 Hz, 1H), 7.11 (dd, J = 8.0, 1.8 Hz, 1H), |
| | | 6.49-6.39 (m, 3H), 4.43 (s, 2H), 3.93-3.81 (m, 3H), 3.74 (s, 2H), 3.44- |
| | | 3.34 (m, 2H), 3.07 (s, 3H), 1.90-1.82 (m, 2H), 1.45-1.30 (m, 14H) |
| 210 | 478.8 | 1H NMR (400 MHz, DMSO): δ (ppm): 7.89 (d, J = 5.2 Hz, 1H), 7.08 (d, J = |
| | | 7.7 Hz, 1H), 6.59 (dd, J = 7.7, 1.7 Hz, 1H), 6.48-6.41 (m, 4H), 4.41 (s, |
| | | 2H), 3.93-3.80 (m, 3H), 3.42-3.35 (m, 2H), 3.10 (s, 2H), 2.71 (s, 3H), |
| | | 1.85 (d, J = 10.6 Hz, 2H), 1.45-1.34 (m, 8H), 1.26 (s, 6H). |
| 211 | 554.5 | 1H NMR (500 MHz, DMSO) δ (ppm): 7.90 (d, J = 5.1 Hz, 1H), 7.67 (d, J = |
| | | 8.1 Hz, 1H), 7.28 (d, J = 1.7 Hz, 1H), 7.17 (dd, J = 8.0, 1.8 Hz, 1H), |
| | | 6.47 (dd, J = 5.3, 1.4 Hz, 1H), 6.44 (t, J = 3.7 Hz, 2H), 4.43 (s, 2H), 4.08 |
| | | (s, 2H), 3.94-3.88 (m, 1H), 3.88-3.82 (m, 2H), 3.39 (td, J = 11.5, 2.2 |
| | | Hz, 2H), 3.02 (s, 3H), 2.34 (dd, J = 11.4, 5.5 Hz, 4H), 2.04 (dd, J = 15.2, |
| | | 7.8 Hz, 2H), 1.90-1.81 (m, 2H), 1.45-1.35 (m, 8H). |
| 212 | 540.4 | 1H NMR (500 MHz, DMSO) δ (ppm): 7.90 (d, J = 5.1 Hz, 1H), 7.30 (d, J = |
| | | 1.7 Hz, 1H), 7.06 (dd, J = 8.1, 1.8 Hz, 1H), 6.96 (d, J = 8.0 Hz, 1H), |
| | | 6.47 (dd, J = 5.3, 1.4 Hz, 1H), 6.44 (t, J = 3.7 Hz, 2H), 4.42 (s, 2H), 4.01 |
| | | (s, 2H), 3.90 (dd, J = 7.4, 3.7 Hz, 1H), 3.88-3.81 (m, 2H), 3.39 (td, J = |
| | | 11.5, 2.2 Hz, 2H), 3.07 (s, 3H), 1.90-1.82 (m, 2H), 1.92-1.81 (m, 2H), |
| | | 1.46-1.36 (m, 8H), 1.19 (dd, J = 6.9, 4.5 Hz, 2H), 1.09 (dd, J = 6.8, 4.4 |
| | | Hz, 2H). |
| 213 | 528.4 | 1H NMR (500 MHz, DMSO) δ (ppm): 7.89 (d, J = 6.0 Hz, 1H), 7.30 (d, J = |
| | | 1.7 Hz, 1H), 7.06 (dd, J = 8.0, 1.8 Hz, 1H), 6.96 (d, J = 8.1 Hz, 1H), |
| | | 6.46 (d, J = 4.4 Hz, 2H), 6.30 (d, J = 7.8 Hz, 1H), 4.42 (s, 2H), 4.10 (dt, J = |
| | | 13.1, 6.5 Hz, 1H), 4.01 (s, 2H), 3.39 (dd, J = 9.2, 5.1 Hz, 1H), 3.25 (d, J = |
| | | 2.3 Hz, 3H), 3.22 (dd, J = 9.2, 6.2 Hz, 1H), 3.07 (s, 3H), 1.40 (d, J = 2.6 |
| | | Hz, 6H), 1.19 (dd, J = 6.9, 4.5 Hz, 2H), 1.11 (d, J = 6.6 Hz, 3H), 1.10- |
| | | 1.06 (m, 2H). |
| 214 | 490.4 | 1H NMR (500 MHz, DMSO) δ (ppm): 7.90 (d, J = 5.2 Hz, 1H), 7.34 (d, J = |
| | | 7.7 Hz, 1H), 6.64 (dd, J = 7.7, 1.8 Hz, 1H), 6.45 (dd, J = 7.7, 6.0 Hz, |
| | | 4H), 4.41 (s, 2H), 3.94-3.89 (m, 1H), 3.89-3.82 (m, 2H), 3.43-3.36 |
| | | (m, 4H), 2.71 (s, 3H), 2.30-2.22 (m, 2H), 2.22-2.14 (m, 2H), 2.03- |
| | | 1.95 (m, 2H), 1.86 (dd, J = 12.4, 2.3 Hz, 2H), 1.46-1.35 (m, 8H). |
| 215 | 525.4 | 1H NMR (400 MHz, DMSO-D6) δ (ppm): 8.80 (br s, 1H), 7.93 (d, J = 6.5 |
| | | Hz, 1H), 7.40 (d, J = 8.0 Hz, 1H), 7.31 (d, J = 1.7 Hz, 1H), 7.13 (dd, J = |
| | | 8.0, 1.7 Hz, 1H), 7.03 (s, 1H), 6.90 (d, J = 6.3 Hz, 1H), 4.64 (s, 2H), 4.33 |
| | | (dt, J = 12.6, 6.3 Hz, 1H), 3.73 (s, 2H)- 3.07 (s, 3H), 2.98-2.82 (m, 2H), |
| | | 1.44 (app d, J- = 2.6 Hz, 6H), 1.36-1.31 (m, 9H). |
| 216 | 552.5 | 1H NMR (500 MHz, DMSO) δ (ppm): 7.95 (d, J = 5.2 Hz, 1H), 7.66 (d, J = |
| | | 8.0 Hz, 1H), 7.28 (d, J = 1.8 Hz, 1H), 7.16 (dd, J = 8.0, 1.8 Hz, 1H), |
| | | 6.74 (d, J = 2.3 Hz, 1H), 6.59 (d, J = 5.2 Hz, 1H), 6.51 (s, 1H), 4.50 (s, |
| | | 2H), 4.07 (s, 2H), 3.86 (d, J = 8.4 Hz, 2H), 3.64 (d, J = 8.1 Hz, 2H), 3.02 |
| | | (s, 3H), 2.38-2.29 (m, 4H), 2.25 (d, J = 2.1 Hz, 1H), 2.07-1.98 (m, 2H), |
| | | 1.75 (s, 2H), 1.40 (s, 6H). |
| 217 | 461.3 | 1H NMR (500 MHz, DMSO): δ (ppm): 7.94 (d, J = 5.3 Hz, 1H), 7.08 (d, J = |
| | | 7.7 Hz, 1H), 6.72 (d, J = 7.0 Hz, 1H), 6.59 (dd, J = 7.7, 1.8 Hz, 1H), |
| | | 6.53 (dd, J = 5.3, 1.4 Hz, 1H), 6.50 (s, 1H), 6.46 (d, J = 1.7 Hz, 1H), 4.43 |
| | | (s, 2H), 4.20-4.10 (m, 1H), 3.10 (s, 2H), 2.85 (dd, J = 16.7, 5.9 Hz, 1H), |
| | | 2.76-2.70 (m, 4H), 1.38 (s, 3H), 1.37 (s, 3H), 1.28-1.24 (m, 9H). |
| 218 | 476.4 | 1H NMR (500 MHz, DMSO): δ (ppm): 7.94 (d, J = 5.2 Hz, 1H), 7.08 (d, J = |
| | | 7.7 Hz, 1H), 6.74 (d, J = 2.3 Hz, 1H), 6.59-6.54 (m, 2H), 6.50 (s, 1H), |
| | | 6.44 (d, J = 1.7 Hz, 1H), 4.47 (s, 2H), 3.87 (d, J = 8.4 Hz, 2H), 3.64 (d, J = |
| | | 8.2 Hz, 2H), 3.10 (s, 2H), 2.71 (s, 3H), 2.25 (d, J = 2.1 Hz, 1H), 1.76 (s, |
| | | 2H), 1.37 (s, 6H), 1.26 (s, 6H). |
| 219 | 544.3 | ¹H NMR (400 MHz, DMSO) δ (ppm): 7.86 (d, J = 5.2 Hz, 1H), 7.37 (d, J = |
| | | 8.0 Hz, 1H), 7.27 (d, J = 1.8 Hz, 1H), 7.08 (dd, J = 8.0, 1.8 Hz, 1H), |
| | | 6.42 (dd, J = 5.3, 1.3 Hz, 1H), 6.37 (s, 1H), 6.26 (d, J = 8.1 Hz, 1H), 4.40 |
| | | (s, 2H), 3.95 (dt, J = 13.7, 6.7 Hz, 1H), 3.71 (s, 2H), 3.35 (d, J = 6.6 Hz, |
| | | 2H), 3.16 (s, 3H), 3.05 (s, 3H), 1.74-1.67 (m, 1H), 1.64-1.57 (m, 1H), 1.38 |
| | | (d, J = 1.4 Hz, 6H), 1.32 (s, 6H), 1.07 (d, J = 6.5 Hz, 3H). |
| 220 | 544.3 | ¹H NMR (400 MHz, DMSO) δ (ppm): 7.91-7.81 (m, 1H), 7.37 (d, J = |
| | | 8.0 Hz, 1H), 7.27 (d, J = 1.8 Hz, 1H), 7.09 (dd, J = 8.0, 1.8 Hz, 1H), 6.43 |
| | | (d, J = 4.0 Hz, 2H), 6.27 (d, J = 7.9 Hz, 1H), 4.40 (s, 2H), 4.05 (dt, J = |
| | | 12.9, 6.5 Hz, 1H), 3.71 (s, 2H), 3.42-3.39 (m, 2H), 3.22-3.20 m, 2H), |
| | | 3.05 (s, 3H), 1.38 (d, J = 1.9 Hz, 6H), 1.32 (s, 6H), 1.13-1.04 (m, 6H). |
| 221 | 537.4 | 1H NMR (400 MHz, DMSO) δ (ppm): 7.92 (d, J = 5.3 Hz, 1H), 7.64 (d, J = |
| | | 8.1 Hz, 1H), 7.26 (d, J = 1.8 Hz, 1H), 7.15 (dd, J = 8.0, 1.8 Hz, 1H), |
| | | 6.72 (s, 1H), 6.57-6.44 (m, 2H), 4.44 (s, 2H), 4.19-4.09 (m, 1H), 4.05 |
| | | (s, 2H), 3.02-2.97 (m, 3H), 2.89-2.78 (m, 1H), 2.76-2.66 (m, 1H), |
| | | 2.35-2.26 (m, 4H), 2.05-1.96 (m, 2H), 1.41-1.34 (m, 6H), 1.28-1.18 |
| | | (m, 3H). |
| 222 | 538.4 | 1H NMR (500 MHz, DMSO) δ (ppm): 7.95 (d, J = 4.9 Hz, 1H), 7.30 (d, J = |
| | | 1.6 Hz, 1H), 7.05 (dd, J = 8.0, 1.8 Hz, 1H), 6.96 (d, J = 8.1 Hz, 1H), |
| | | 6.73 (d, J = 2.4 Hz, 1H), 6.59 (dd, J = 5.2, 1.4 Hz, 1H), 6.51 (s, 1H), 4.49 |
| | | (s, 2H), 4.01 (s, 2H), 3.86 (d, J = 8.4 Hz, 2H), 3.64 (d, J = 8.2 Hz, 2H), |
| | | 3.07 (s, 3H), 2.25 (d, J = 2.2 Hz, 1H), 1.76 (s, 2H), 1.40 (s, 6H), 1.19 (dd, |
| | | J = 6.9, 4.5 Hz, 2H), 1.09 (dd, J = 6.8, 4.4 Hz, 2H). |
| 223 | 526.4 | 1H NMR (500 MHz, DMSO) δ (ppm): 7.92 (d, J = 5.4 Hz, 1H), 7.30 (d, J = |
| | | 1.7 Hz, 1H), 7.06 (dd, J = 8.0, 1.8 Hz, 1H), 6.96 (d, J = 8.0 Hz, 1H), |
| | | 6.71 (d, J = 6.2 Hz, 1H), 6.51 (dd, J = 5.3, 1.4 Hz, 1H), 6.47 (s, 1H), 4.44 |
| | | (s, 2H), 4.34 (dd, J = 8.6, 4.8 Hz, 1H), 4.01 (s, 2H), 3.84 (ddd, J = 20.4, |
| | | 12.1, 6.6 Hz, 2H), 3.71 (td, J = 8.1, 5.6 Hz, 1H), 3.49 (dd, J = 8.7, 3.9 Hz, |
| | | 1H), 3.07 (s, 3H), 2.20-2.10 (m, 1H), 1.81-1.72 (m, 1H), 1.40 (s, 6H), |
| | | 1.19 (dd, J = 6.9, 4.5 Hz, 2H), 1.09 (dd, J = 6.8, 4.4 Hz, 2H). |
| 224 | 574.4 | 1H NMR (400 MHz, DMSO) δ (ppm): 7.89 (d, J = 5.8 Hz, 1H), 7.39 (d, J = |
| | | 8.0 Hz, 1H), 7.29 (d, J = 1.8 Hz, 1H), 7.10 (dd, J = 8.0, 1.8 Hz, 1H), |
| | | 6.45 (d, J = 5.2 Hz, 1H), 6.45 (s, 1H), 6.30 (d, J = 7.8 Hz, 1H), 4.42 (s, |
| | | 2H), 4.07 (dt, J = 12.9, 6.6 Hz, 1H), 3.73 (s, 2H), 3.54-3.39 (m, 4H), |
| | | 3.48-3.44 (m, 1H), 3.26 (dd, J = 9.3, 6.5 Hz, 1H), 3.23 (s, 3H), 3.07 (s, |
| | | 3H), 1.40 (s, 6H), 1.34 (s, 6H), 1.11 (d, J = 6.6 Hz, 3H). |
| 225 | 568.3 | 1H NMR (400 MHz, DMSO) δ (ppm): 7.87 (d, J = 5.2 Hz, 1H), 7.66 (d, J = |
| | | 8.1 Hz, 1H), 7.28 (d, J = 1.8 Hz, 1H), 7.16 (dd, J = 8.0, 1.8 Hz, 1H), |
| | | 6.46-6.35 (m, 3H), 4.42 (s, 2H), 4.07 (s, 2H), 4.01-3.86 (m, 1H), 3.77- |
| | | 3.66 (m, 2H), 3.64-3.52 (m, 1H), 3.44-3.34 (m, 1H), 3.02 (s, 3H), |
| | | 2.37-2.22 (m, 5H), 2.06-1.97 (m, 2H), 1.97-1.85 (m, 1H), 1.68-1.51 |
| | | (m, 1H), 1.40 (s, 3H), 1.40 (s, 3H), 1.07 (dd, J = 16.6, 6.4 Hz, 3H). |
| 226 | 554.3 | 1H NMR (400 MHz, DMSO) δ 7.88 (d, J = 5.3 Hz, 1H), 7.64 (d, J = 8.1 |
| | | Hz, 1H), 7.25 (d, J = 1.8 Hz, 1H), 7.14 (dd, J = 8.0, 1.8 Hz, 1H), 6.61- |
| | | 6.36 (m, 2H), 4.42 (s, 2H), 4.05 (s, 2H), 3.79-3.64 (m, 2H), 3.59 (dd, J = |
| | | 14.9, 7.9 Hz, 1H), 3.41 (dd, J = 8.5, 5.5 Hz, 1H), 3.24-3.09 (m, 2H), |
| | | 3.00 (s, 3H), 2.31 (t, J = 8.2 Hz, 4H), 2.08-1.86 (m, 3H), 1.58-1.52 (m |
| | | 1H), 1.38 (s, 6H). *missing 1 proton under DMSO peak |
| 227 | 522.4 | ¹H NMR (400 MHz, DMSO) δ (ppm): 7.87 (d, J = 5.2 Hz, 1H), 7.05 (d, J = |
| | | 7.7 Hz, 1H), 6.53 (dd, J = 7.7, 1.7 Hz, 1H), 6.50-6.36 (m, 4H), 4.39 (s, |
| | | 2H), 3.88-3.77 (m, 3H), 3.51 (t, J = 5.7 Hz, 2H), 3.36 (td, J = 11.4, 2.0 |
| | | Hz, 2H), 3.26 (s, 3H), 3.24-3.19 (m, 4H), 1.88-1.73 (m, 2H), 1.42- |
| | | 1.28 (m, 8H), 1.23 (s, 6H). |
| 228 | 586.4 | ¹H NMR (400 MHz, DMSO) δ (ppm): 7.87 (d, J = 5.3 Hz, 1H), 7.35 (d, J = |
| | | 8.0 Hz, 1H), 7.25 (d, J = 1.8 Hz, 1H), 7.04 (dd, J = 8.0, 1.8 Hz, 1H), |
| | | 6.50-6.32 (m, 3H), 4.41 (s, 2H), 3.88-3.81 (m, 3H), 3.73 (s, 2H), 3.64 (t, |
| | | J = 5.4 Hz, 3H), 3.54 (t, J = 5.4 Hz, 3H), 3.02 (s, 3H), 1.87-1.77 (m, |
| | | 2H), 1.41-1.35 (m, 8H), 1.31 (s, 6H). |
| 229 | 463.3 | 1H NMR (400 MHz, DMSO-D6) δ (ppm): 7.94 (d, J = 5.1 Hz, 1H), 7.30 |
| | | (d, J = 7.9 Hz, 1H), 6.88 (dd, J = 7.9, 1.8 Hz, 1H), 6.81 (d, J = 1.7 Hz, |
| | | 1H), 6.75 (d, J = 2.2 Hz, 1H), 6.57 (dd, J = 5.2, 1.3 Hz, 1H), 6.50 (s, 1H), |
| | | 4.48 (s, 2H), 4.28 (s, 2H), 3.86 (d, J = 8.4 Hz, 2H), 3.63 (d, J- = 8.2 Hz, |
| | | 2H), 2.26-2.23 (m, 1H), 1.75 (s, 2H), 1.37 (s, 6H), 1.32 (s, 6H). |
| 230 | 523.3 | 1H NMR (500 MHz, DMSO) δ (ppm): 7.94 (d, J = 5.3 Hz, 1H), 7.30 (d, J = |
| | | 1.8 Hz, 1H), 7.06 (dd, J = 8.0, 1.8 Hz, 1H), 6.96 (d, J = 8.1 Hz, 1H), |
| | | 6.72 (d, J = 7.0 Hz, 1H), 6.58-6.53 (m, 1H), 6.52 (s, 1H), 4.45 (s, 2H), |
| | | 4.16 (dt, J = 12.5, 6.4 Hz, 1H), 4.01 (s, 2H), 3.07 (s, 3H), 2.85 (dd, J = |
| | | 16.7, 5.9 Hz, 1H), 2.73 (dd, J = 16.7, 4.8 Hz, 1H), 1.41 (d, J = 3.0 Hz, |
| | | 6H), 1.26 (d, J = 6.7 Hz, 3H), 1.22-1.16 (m, 2H), 1.09 (dd, J = 6.8, 4.4 |
| | | Hz, 2H). |
| 231 | 528.4 | 1H NMR (500 MHz, DMSO) δ (ppm): 8.01 (s, 1H), 7.95 (d, J = 5.2 Hz, |
| | | 1H), 6.96 (dd, J = 8.0, 1.8 Hz, 1H), 6.91 (d, J = 8.0 Hz, 1H), 6.75 (s, 1H), |
| | | 6.58 (d, J = 5.1 Hz, 1H), 6.51 (s, 1H), 4.48 (s, 2H), 4.37 (s, 2H), 3.86 (d, J = |
| | | 8.4 Hz, 2H), 3.63 (d, J = 8.2 Hz, 2H), 2.26 (s, 1H), 1.88 (s, 1H), 1.76 (s, |
| | | 2H), 1.39 (s, 6H), 1.19 (t, J = 5.7 Hz, 2H), 1.12 (dd, J = 6.7, 4.5 Hz, 2H), |
| | | 0.88 (t, J = 5.8 Hz, 4H). |
| 232 | 560.2 | ¹H NMR (400 MHz, DMSO) δ (ppm): 7.91 (d, J = 5.3 Hz, 1H), 7.64 (d, J = |
| | | 8.1 Hz, 1H), 7.26 (d, J = 1.8 Hz, 1H), 7.15 (dd, J = 8.0, 1.8 Hz, 1H), |
| | | 6.75 (t, J = 5.7 Hz, 1H), 6.50 (dd, J = 8.6, 3.2 Hz, 2H), 4.42 (s, 2H), 4.05 |
| | | (s, 2H), 3.28-3.18 (m, 2H), 3.00 (s, 3H), 2.33-2.29 (m, 4H), 2.10-1.90 |
| | | (m, 3H), 1.57-1.48 (m, 1H), 1.38 (s, 6H), 1.29-1.21 (m, 1H). |
| 233 | 594.3 | ¹H NMR (400 MHz, DMSO) δ (ppm): 7.84 (d, J = 5.3 Hz, 1H), 7.65 (d, J = |
| | | 8.0 Hz, 1H), 7.26 (d, J = 1.8 Hz, 1H), 7.15 (dd, J = 8.0, 1.8 Hz, 1H), |
| | | 6.46 (s, 1H), 6.42 (dd, J = 5.3, 1.2 Hz, 1H), 6.35 (t, J = 6.1 Hz, 1H), 4.40 |
| | | (s, 2H), 4.05 (s, 2H), 3.61 (d, J = 3.9 Hz, 1H), 3.54 (s, 2H), 3.03 (d, J = |
| | | 6.1 Hz, 2H), 3.00 (s, 3H), 2.33-2.29 (m, 4H), 2.06-1.95 (m, 2H), 1.88- |
| | | 1.75 (m, 2H), 1.56-1.45 (m, 6H), 1.38 (s, 6H). |
| 234 | 542.2 | 1H NMR (400 MHz, DMSO) δ (ppm): 8.00-7.90 (m, 2H), 7.64 (d, J = |
| | | 8.0 Hz, 1H), 7.08 (d, J = 8.0 Hz, 1H), 6.74 (s, 1H), 6.65-6.56 (m, 1H), |
| | | 6.50 (s, 1H), 4.48 (s, 4H), 3.86 (d, J = 8.3 Hz, 2H), 3.63 (d, J = 7.8 Hz, |
| | | 2H), 2.44-2.28 (m, 4H), 2.25 (s, 1H), 2.04 (m, 3H), 1.75 (s, 2H), 1.39 (s, |
| | | 6H), 0.93-0.84 (m, 4H). |
| 235 | 556.4 | 1H NMR (400 MHz, DMSO-D6) δ (ppm): 7.87 (d, J = 5.3 Hz, 1H), 7.66 |
| | | (d, J = 8.1 Hz, 1H), 7.28 (d, J = 1.7 Hz, 1H), 7.17 (dd, J = 8.0, 1.8 Hz, |
| | | 1H), 6.60 (s, 1H), 6.44 (dd, J = 5.3, 1.2 Hz, 1H), 6.23 (t, J = 5.7 Hz, 1H), |
| | | 4.42 (s, 2H-, 4.07 (s, 2H), 3.3-3.31 (overlapped m, 2H), 3.12 (s, 3H)- |
| | | 3.02 (s, 3H), 2.36-2.29 (m, 4H), 2.09-1.97 (m, 2H), 1.40 (s, 6H), 1.11 |
| | | (s, 6H). |
| 236 | 530.2 | 1H NMR (400 MHz, DMSO) δ (ppm): 7.98 (s, 1H), 7.91 (d, J = 5.3 Hz, |
| | | 1H), 7.64 (d, J = 8.0 Hz, 1H), 7.09 (dd, J = 8.0, 1.9 Hz, 1H), 6.73 (d, J = |
| | | 6.0 Hz, 1H), 6.49 (dd, J = 5.3, 1.1 Hz, 1H), 6.46 (s, 1H), 4.47 (s, 2H), 4.43 |
| | | (s, 2H), 4.33 (dtt, J = 7.9, 6.2, 4.1 Hz, 1H), 3.85 (dd, J = 8.8, 5.9 Hz, 1H), |
| | | 3.82-3.78 (m, 1H), 3.70 (td, J = 8.1, 5.7 Hz, 1H), 3.49 (dd, J = 8.7, 3.9 |
| | | Hz, 1H), 2.43-2.28 (m, 4H), 2.20-2.09 (m, 1H), 2.09-1.95 (m, 3H), |
| | | 1.81-1.72 (m, 1H), 1.39 (s, 6H), 0.93-0.85 (m, 4H). |
| 237 | 544.2 | 1H NMR (400 MHz, DMSO) δ (ppm): 7.98 (s, 1H), 7.89 (d, J = 5.3 Hz, |
| | | 1H), 7.64 (d, J = 8.0 Hz, 1H), 7.09 (dd, J = 8.0, 1.9 Hz, 1H), 6.51 (s, 1H), |
| | | 6.46 (d, J = 5.4 Hz, 1H), 6.44 (s, 1H), 4.47 (s, 2H), 4.42 (s, 2H), 3.95- |
| | | 3.79 (m, 3H), 3.38 (td, J = 11.4, 1.9 Hz, 2H), 2.44-2.24 (m, 4H), 2.10- |
| | | 1.95 (m, 3H), 1.90-1.81 (m, 2H), 1.46-1.31 (m, 8H), 0.91-0.84 (m, 4H). |
| 238 | 542.4 | 1H NMR (400 MHz, DMSO-D6) δ 7.89 (d, J = 5.3 Hz, 1H), 7.66 (d, J = |
| | | 8.0 Hz, 1H), 7.28 (d, J = 1.8 Hz, 1H), 7.17 (dd, J = 8.0, 1.8 Hz, 1H)- 6.51 |
| | | (s, 1H), 6.48-6.43 (m, 2H), 4.43 (s, 2H), 4.07 (s, 2H), 3.48-3.42 (m, |
| | | 1H), 3.27 (t, J = 5.6 Hz, 2H), 3.25 (s, 3H), 3.01 (s, 3H), 2.37-2.28 (m, |
| | | 4H), 2.03 (dt, J = 8.9, 4.6 Hz, 2H), 1.40 (s, 6H), 1.08 (d, J = 6.2 Hz, 3H) |
| 239 | 542.4 | 1H NMR (400 MHz, DMSO-D6) δ 7.89 (d, J = 5.3 Hz, 1H), 7.66 (d, J = |
| | | 8.1 Hz, 1H), 7.28 (d, J = 1.7 Hz, 1H), 7.17 (dd, J = 8.0, 1.8 Hz, 1H)- 6.51 |
| | | (s, 1H), 6.48-6.43 (m, 2H), 4.43 (s, 2H), 4.07 (s, 2H), 3.50-3.41 (m, |
| | | 1H), 3.27 (dd, J = 8.6, 3.3 Hz, 2H), 3.25 (s, 3H), 3.01 (s, 3H), 2.37-2.29 |
| | | (m, 4H), 2.08-1.97 (m, 2H), 1.40 (s, 6H), 1.08 (d, J = 6.2 Hz, 3H). |
| 240 | 528.3 | 1H NMR (400 MHz, DMSO) δ 7.99-7.85 (m, 1H), 7.64 (d, J = 8.0 Hz, |
| | | 1H), 7.25 (d, J = 1.7 Hz, 1H), 7.15 (dd, J = 8.0, 1.8 Hz, 1H), 6.77 (d, J = |
| | | 3.0 Hz, 1H), 6.57 (d, J = 4.4 Hz, 2H), 4.85-4.60 (m, 1H), 4.46 (s, 2H), |
| | | 4.05 (s, 2H), 3.00 (s, 3H), 2.59 (dd, J = 8.5, 4.4 Hz, 1H), 2.33-2.29 (m, |
| | | 4H), 2.09-1.92 (m, 2H), 1.38 (2 s, 6H), 1.11-1.02 (m, 1H), 0.95-0.77 |
| | | (m, 1H). |
| 241 | 560.3 | 1H NMR (400 MHz, DMSO) δ 7.92 (d, J = 5.2 Hz, 1H), 7.39 (d, J = 8.0 |
| | | Hz, 1H), 7.30 (d, J = 1.7 Hz, 1H), 7.11 (dd, J = 8.0, 1.8 Hz, 1H), 6.72 (d, J = |
| | | 7.4 Hz, 1H), 6.52 (dd, J = 7.3, 2.1 Hz, 1H), 6.51 (s, 1H), 4.46 (dtd, J = |
| | | 18.4, 7.2, 3.7 Hz, 1H), 4.44 (s, 2H), 4.18 (dd, J = 9.5, 5.5 Hz, 1H), 3.93 |
| | | (ddd, J = 15.9, 11.8, 3.7 Hz, 1H), 3.82-3.74 (m, 1H), 3.73 (s, 2H), 3.56- |
| | | 3.39 (m, 2H), 3.07 (s, 3H), 2.10-2.00 (m, 1H), 1.55-1.42 (m, 1H), 1.40 |
| | | (d, J = 2.3 Hz, 6H), 1.34 (s, 6H). |
| 242 | 575.3 | 1H NMR (400 MHz, DMSO) δ 8.56 (d, J = 2.1 Hz, 1H), 8.38 (dd, J = 4.7, |
| | | 1.6 Hz, 1H), 7.85 (d, J = 5.3 Hz, 1H), 7.72 (dt, J = 7.9, 1.9 Hz, 1H), 7.66 |
| | | (d, J = 8.1 Hz, 1H), 7.32-7.26 (m, 2H), 7.17 (dd, J = 8.0, 1.8 Hz, 1H), |
| | | 7.04 (d, J = 7.6 Hz, 1H), 6.53-6.41 (m, 2H), 5.02 (p, J = 6.9 Hz, 1H), |
| | | 4.47-4.37 (m, 2H), 4.07 (s, 2H), 3.02 (s, 3H), 2.39-2.26 (m, 4H), 2.11- |
| | | 1.97 (m, 2H), 1.44 (d, J = 7.0 Hz, 3H), 1.37 (s, 3H), 1.31 (s, 3H). |
| 243 | 575.3 | 1H NMR (400 MHz, DMSO) δ 8.56 (d, J = 2.1 Hz, 1H), 8.38 (dd, J = 4.7, |
| | | 1.6 Hz, 1H), 7.85 (d, J = 5.2 Hz, 1H), 7.72 (dt, J = 7.9, 1.9 Hz, 1H), 7.66 |
| | | (d, J = 8.1 Hz, 1H), 7.31-7.26 (m, 2H), 7.18 (dd, J = 8.0, 1.8 Hz, 1H), |
| | | 7.04 (d, J = 7.6 Hz, 1H), 6.50-6.44 (m, 2H), 5.02 (p, J = 6.9 Hz, 1H), |
| | | 4.48-4.34 (m, 2H), 4.07 (s, 2H), 3.02 (s, 3H), 2.38-2.29 (m, 4H), 2.06- |
| | | 1.96 (m, 2H), 1.44 (d, J = 7.0 Hz, 3H), 1.37 (s, 3H), 1.31 (s, 3H). |
| 244 | 561.4 | 1H NMR (400 MHz, DMSO) δ 8.53 (d, J = 1.7 Hz, 1H), 8.42 (dd, J = 4.8, |
| | | 1.6 Hz, 1H), 7.91 (d, J = 5.3 Hz, 1H), 7.70 (dt, J = 7.8, 1.9 Hz, 1H), 7.66 |
| | | (d, J = 8.0 Hz, 1H), 7.31 (ddd, J = 7.8, 4.8, 0.7 Hz, 1H), 7.28 (d, J = 1.8 |
| | | Hz, 1H), 7.16 (dd, J = 8.0, 1.8 Hz, 1H), 7.10 (t, J = 6.0 Hz, 1H), 6.53 (dd, |
| | | J = 5.3, 1.3 Hz, 1H), 6.50 (s, 1H), 4.48 (d, J = 6.0 Hz, 2H), 4.44 (s, 2H), |
| | | 4.07 (s, 2H), 3.02 (s, 3H), 2.39-2.27 (m, 4H), 2.08-1.94 (m, 2H), 1.36 |
| | | (s, 6H). |
| 245 | 538.3 | 1H NMR (400 MHz, DMSO) δ 8.23 (s, 1H), 7.91 (d, J = 5.7 Hz, 1H), 7.50 |
| | | (s, 1H), 7.37 (d, J = 8.0 Hz, 1H), 7.27 (d, J = 1.7 Hz, 1H), 7.08 (dd, J = |
| | | 8.0, 1.8 Hz, 1H), 6.74 (s, 1H), 6.49 (d, J = 5.3 Hz, 2H), 6.11 (d, J = 2.0 |
| | | Hz, 1H), 4.42 (s, 2H), 4.39 (d, J = 5.6 Hz, 2H), 3.71 (s, 2H), 3.04 (s, 3H), |
| | | 1.35 (s, 6H), 1.32 (s, 6H). |
| 246 | 552.3 | 1H NMR (400 MHz, DMSO) δ 7.97-7.80 (m, 1H), 7.52 (d, J = 2.1 Hz, |
| | | 1H), 7.37 (d, J = 8.0 Hz, 1H), 7.28 (d, J = 1.8 Hz, 1H), 7.09 (dd, J = 8.0, |
| | | 1.8 Hz, 1H), 6.72 (t, J = 5.7 Hz, 1H), 6.51-6.40 (m, 2H), 6.07 (d, J = 2.2 |
| | | Hz, 1H), 4.41 (s, 2H), 4.32 (d, J = 5.7 Hz, 2H), 3.73 (s, 3H), 3.71 (s, 2H), |
| | | 3.04 (s, 3H), 1.34 (s, 6H), 1.32 (s, 6H). |
| 247 | 475.3 | 1H NMR (400 MHz, DMSO) δ 8.40 (s, 1H), 7.90 (d, J = 5.2 Hz, 1H), 7.37 |
| | | (d, J = 8.0 Hz, 1H), 7.27 (d, J = 1.7 Hz, 1H), 7.08 (dd, J = 8.0, 1.8 Hz, |
| | | 1H), 6.46 (d, J = 5.3 Hz, 1H), 6.39 (d, J = 7.4 Hz, 2H), 4.42 (s, 2H), 3.71 |
| | | (s, 2H), 3.05 (s, 3H), 1.37 (s, 6H), 1.32 (s, 6H). formate salt |
| 248 | 568.3 | 1H NMR (400 MHz, DMSO) δ 7.87 (d, J = 5.2 Hz, 1H), 7.36 (d, J = 8.0 |
| | | Hz, 1H), 7.32 (d, J = 1.8 Hz, 1H), 7.07 (dd, J = 8.0, 1.8 Hz, 1H), 6.45-6.41 |
| | | (m, 3H), 4.41 (s, 2H), 3.88-3.79 (m, 3H), 3.79 (s, 2H), 3.40-3.37 (m, |
| | | 2H), 2.82-2.71 (m, 1H), 1.83 (d, J = 10.1 Hz, 2H), 1.41-1.35 (m, 8H), |
| | | 1.33 (s, 6H), 1.13-1.08 (m, 2H), 0.99-0.93 (m, 2H). |
| 249 | 463.4 | 1H NMR (400 MHz, DMSO-D6) δ 7.94 (d, J = 5.2 Hz, 1H), 7.26 (d, J = |
| | | 7.9 Hz, 1H), 6.80 (dd, J = 7.—, 1.7 Hz, 1H), 6.75-6.71 (m, 2H), 6.57 (dd, |
| | | J = 5.2, 1.1 Hz, 1H), 6.50 (s, 1H), 4.47 (s, 2H), 3.86 (d, J = 8.4 Hz, 2H), |
| | | 3.63 (d, J = 8.2 Hz, 2H)- 3.04 (s, 2H), 2.26-2.23 (m, 1H), 1.75 (s, 2H), |
| | | 1.43 (s, 6H), 1.37 (s, 6H). Contains ~10% Compound 229 |
| 250 | 483.4 | 1H NMR (500 MHz, DMSO) δ 7.96 (d, J = 5.2 Hz, 1H), 7.91 (d, J = 8.3 |
| | | Hz, 1H), 7.64 (dd, J = 12.8, 4.5 Hz, 2H), 6.73 (d, J = 2.2 Hz, 1H), 6.62 (d, |
| | | J = 5.2 Hz, 1H), 6.54 (s, 1H), 4.51 (s, 2H), 3.88 (d, J = 8.3 Hz, 2H), 3.70- |
| | | 3.61 (m, 4H), 3.40 (dd, J = 15.7, 8.8 Hz, 2H), 2.29-2.27 (m, 1H), 1.77 (s, |
| | | 2H), 1.43 (s, 6H). |
| 251 | 511.9 | 1H NMR (400 MHz, DMSO) δ 7.89 (d, J = 5.2 Hz, 1H), 7.33 (d, J = 8.0 |
| | | Hz, 1H), 7.25 (d, J = 1.7 Hz, 1H), 7.02 (dd, J = 8.0, 1.8 Hz, 1H), 6.68 (d, J = |
| | | 2.0 Hz, 1H), 6.54 (d, J = 5.2 Hz, 1H), 6.46 (s, 1H), 4.44 (s, 2H), 3.94 (t, |
| | | J = 8.5 Hz, 2H), 3.80 (d, J = 8.4 Hz, 2H), 3.58 (d, J = 8.2 Hz, 2H), 3.10 (t, |
| | | J = 8.4 Hz, 2H), 2.97 (s, 3H), 2.20 (d, J = 2.1 Hz, 1H), 1.70 (s, 2H), 1.35 |
| | | (s, 6H). |
| 252 | 502 | 1H NMR (500 MHz, DMSO) δ 8.02 (s, 1H), 7.95 (d, J = 5.2 Hz, 1H), 7.34 |
| | | (d, J = 7.9 Hz, 1H), 6.99 (dd, J = 7.9, 1.9 Hz, 1H), 6.74 (d, J = 2.3 Hz, |
| | | 1H), 6.58 (dd, J = 5.2, 1.1 Hz, 1H), 6.51 (s, 1H), 4.48 (s, 2H), 4.36 (t, J = |
| | | 8.3 Hz, 2H), 3.86 (d, J = 8.4 Hz, 2H), 3.63 (d, J = 8.2 Hz, 2H), 3.23 (t, J = |
| | | 8.5 Hz, 2H), 2.26 (d, J = 2.1 Hz, 1H), 2.00-1.94 (m, 1H), 1.75 (s, 2H), |
| | | 1.39 (s, 6H), 0.88 (d, J = 6.3 Hz, 4H). |
| 253 | 505 | 1H NMR (500 MHz, DMSO) δ 7.95 (d, J = 5.3 Hz, 1H), 7.78 (d, J = 1.9 |
| | | Hz, 1H), 7.27 (d, J = 7.9 Hz, 1H), 6.87 (dd, J = 7.9, 1.9 Hz, 1H), 6.78 (s, |
| | | 1H), 6.59 (d, J = 5.2 Hz, 1H), 6.52 (s, 1H), 6.37 (s, 2H), 4.50 (s, 2H), 3.87 |
| | | (d, J = 8.4 Hz, 2H), 3.70 (s, 2H), 3.64 (d, J = 8.3 Hz, 2H), 2.29-2.23 (m, |
| | | 1H), 1.80-1.72 (m, 2H), 1.40 (s, 6H), 1.32 (s, 6H). |
| 254 | 419.4 | 1H NMR (500 MHz, DMSO) δ 7.93 (d, J = 8.7 Hz, 1H), 7.88-7.84 (m, |
| | | 2H), 7.43 (dd, J = 8.8, 1.7 Hz, 1H), 6.54 (dd, J = 5.3, 1.4 Hz, 1H), 6.47 (s, |
| | | 1H), 5.87 (s, 2H), 4.47 (s, 2H), 1.44 (s, 6H). |
| 255 | 433.4 | 1H NMR (500 MHz, DMSO) δ 7.98 (dd, J = 1.6, 0.7 Hz, 1H), 7.94 (d, J = |
| | | 8.7 Hz, 1H), 7.87 (d, J = 5.2 Hz, 1H), 7.44 (dd, J = 8.7, 1.6 Hz, 1H), 6.54 |
| | | (dd, J = 5.3, 1.5 Hz, 1H), 6.47 (s, 1H), 5.89 (s, 2H), 4.47 (s, 2H), 4.20 (s, |
| | | 3H), 1.45 (s, 6H). |
| 256 | 475.3 | 1H NMR (500 MHz, DMSO) δ 7.98 (dd, J = 1.6, 0.7 Hz, 1H), 7.94 (d, J = |
| | | 8.7 Hz, 1H), 7.92 (d, J = 5.2 Hz, 1H), 7.44 (dd, J = 8.7, 1.6 Hz, 1H), 6.48 |
| | | (dd, J = 5.3, 1.4 Hz, 1H), 6.45 (s, 1H), 6.28 (d, J = 7.6 Hz, 1H), 4.47 (s, |
| | | 2H), 4.20 (s, 3H), 4.03-3.95 (m, 1H), 1.46 (s, 6H), 1.14 (d, J = 6.4 Hz, 6H). |
| 257 | 434.4 | 1H NMR (500 MHz, MeOD) δ 7.79 (d, J = 5.4 Hz, 1H), 7.52 (d, J = 8.4 |
| | | Hz, 1H), 7.26 (d, J = 1.4 Hz, 1H), 6.95 (s, 1H), 6.91 (dd, J = 8.4, 1.8 Hz, |
| | | 1H), 6.47 (dd, J = 5.5, 1.5 Hz, 1H), 6.43 (s, 1H), 4.44 (s, 2H), 3.86 (dt, J = |
| | | 12.8, 6.5 Hz, 1H), 3.68 (s, 3H), 2.73-2.63 (m, 2H), 1.39 (s, 6H), 1.22 (t, |
| | | J = 7.5 Hz, 4H), 1.11 (d, J = 6.4 Hz, 6H). |
| 258 | 433.1 | 1H NMR (500 MHz, DMSO) δ 12.77 (s, 1H), 7.90 (d, J = 5.2 Hz, 1H), |
| | | 7.86 (d, J = 8.6 Hz, 1H), 7.53 (d, J = 1.0 Hz, 1H), 7.11 (dd, J = 8.6, 1.7 |
| | | Hz, 1H), 6.46 (dd, J = 5.3, 1.4 Hz, 1H), 6.43 (s, 1H), 6.31 (d, J = 7.5 Hz, |
| | | 1H), 4.45 (s, 2H), 4.02-3.95 (m, 1H), 2.31 (ddd, J = 16.8, 8.3, 5.1 Hz, |
| | | 1H), 1.43 (s, 6H), 1.13 (d, J = 6.4 Hz, 6H), 1.05-0.93 (m, 4H). |
| 259 | 443.3 | 1H NMR (400 MHz, DMSO) δ (ppm): 7.88 (d, J = 6.0 Hz, 1H), 6.47- |
| | | 6.39 (m, 2H), 6.31 (d, J = 7.9 Hz, 1H), 6.24 (s, 1H), 4.41 (s, 2H), 4.15- |
| | | 4.00 (m, 1H), 3.58 (s, 3H), 3.36 (dd, J = 9.2, 5.1 Hz, 1H), 3.23 (s, 3H), |
| | | 3.19 (dd, J = 9.2, 6.2 Hz, 1H), 1.41 (s, 3H), 1.40 (s, 3H), 1.23 (s, 9H), 1.09 |
| | | (d, J = 6.6 Hz, 3H). |
| 260 | 455.4 | 1H NMR (400 MHz, DMSO) δ 7.88 (d, J = 5.2 Hz, 1H), 6.50-6.42 (m, |
| | | 2H), 6.41 (s, 1H), 6.24 (s, 1H), 4.41 (s, 2H), 3.92-3.78 (m, 3H), 3.58 (s, |
| | | 3H), 3.43-3.33 (m, 2H), 1.84 (dd, J = 12.4, 2.2 Hz, 2H), 1.45-1.31 (m, |
| | | 8H), 1.23 (s, 9H). |
| 261 | 441.4 | 1H NMR (400 MHz, DMSO) δ 7.89 (d, J = 5.3 Hz, 1H), 6.69 (d, J = 5.9 |
| | | Hz, 1H), 6.47 (dd, J = 5.3, 1.3 Hz, 1H), 6.43 (s, 1H), 6.22 (s, 1H), 4.41 (s, |
| | | 2H), 4.34-4.26 (m, 1H), 3.86-3.74 (m, 2H), 3.71-3.62 (m, 1H), 3.57 |
| | | (s, 3H), 3.46 (dd, J = 8.8, 3.9 Hz, 1H), 2.12 (ddd, J = 14.8, 12.6, 7.6 Hz, |
| | | 1H), 1.79-1.68 (m, 1H), 1.38 (d, J = 8.0 Hz, 6H), 1.21 (s, 9H). |
| 262 | 438.4 | 1H NMR (400 MHz, DMSO) δ 7.93 (d, J = 5.3 Hz, 1H), 6.70 (d, J = 7.0 |
| | | Hz, 1H), 6.52 (dd, J = 5.3, 1.4 Hz, 1H), 6.49 (s, 1H), 6.24 (s, 1H), 4.43 (s, |
| | | 2H), 4.18-4.08 (m, 1H), 3.59 (s, 3H), 2.83 (dd, J = 16.7, 5.8 Hz, 1H), |
| | | 2.71 (dd, J = 16.7, 4.9 Hz, 1H), 1.41 (s, 3H), 1.40 (s, 3H), 1.25 (d, J = 6.7 |
| | | Hz, 3H), 1.23 (s, 9H). |
| 263 | 409.5 | 1H NMR (400 MHz, DMSO) δ (ppm): 7.87 (d, J = 5.3 Hz, 1H), 7.54- |
| | | 7.45 (m, 2H), 7.38-7.27 (m, 2H), 6.41 (dd, J = 5.3, 1.3 Hz, 1H), 6.38 (s, |
| | | 1H), 6.27 (d, J = 7.6 Hz, 1H), 4.40 (s, 2H), 4.01-3.88 (m, 1H), 1.37 (s, |
| | | 6H), 1.29 (s, 9H), 1.10 (d, J = 6.4 Hz, 6H). |
| 264 | 449.6 | 1H NMR (500 MHz, DMSO) δ (ppm): 7.88 (d, J = 5.2 Hz, 1H), 7.55- |
| | | 7.50 (m, 2H), 7.38-7.30 (m, 2H), 6.44-6.41 (m, 2H), 6.30 (d, J = 7.7 |
| | | Hz, 1H), 4.42 (s, 2H), 3.71-3.60 (m, 1H), 1.93-1.87 (m, 2H), 1.73- |
| | | 1.67 (m, 2H), 1.62-1.55 (m, 1H), 1.40 (s, 6H), 1.32 (s, 9H), 1.30-1.26 |
| | | (m, 2H), 1.20-1.12 (m, 3H). |
| 265 | 439.4 | 1H NMR (400 MHz, DMSO) δ (ppm): 8.06-7.67 (m, 1H), 7.59-7.38 |
| | | (m, 2H), 7.38-7.15 (m, 2H), 6.43 (d, J = 4.1 Hz, 2H), 6.30 (d, J = 7.9 Hz, |
| | | 1H), 4.40 (s, 2H), 4.11-4.05(m, 1H), 3.23 (s, 3H), 1.37 (d, J = 2.1 Hz, 6H), |
| | | 1.29 (s, 9H), 1.09 (d, J = 6.6 Hz, 3H). |
| 266 | 451.4 | ¹H NMR (400 MHz, DMSO) δ (ppm): 7.87 (d, J = 5.2 Hz, 1H), 7.54- |
| | | 7.45 (m, 2H), 7.37-7.27 (m, 2H), 6.48-6.37 (m, 3H), 4.41 (s, 2H), 3.89- |
| | | 3.82 (m, 3H), 3.40-3.36 (m, 2H), 1.84 (d, J = 10.4 Hz, 2H), 1.37 (s, 8H), |
| | | 1.29 (s, 9H). |
| 267 | 410.4 | 1H NMR (400 MHz, DMSO) δ (ppm): 8.59-8.53 (m, 1H), 7.87 (d, J = |
| | | 5.3 Hz, 1H), 7.80 (dd, J = 8.5, 2.6 Hz, 1H), 7.57 (dd, J = 8.5, 0.4 Hz, 1H), |
| | | 6.43 (dd, J = 5.3, 1.3 Hz, 1H), 6.38 (s, 1H), 6.25 (d, J = 7.6 Hz, 1H), 4.42 |
| | | (s, 2H), 4.03-3.88 (m, 1H), 1.39 (s, 6H), 1.32 (s, 9H), 1.10 (d, J = 6.4 |
| | | Hz, 6H). |
| 268 | 439.4 | 1H NMR (400 MHz, DMSO) δ 7.87 (d, J = 5.8 Hz, 1H), 7.50 (d, J = 8.6 |
| | | Hz, 2H), 7.32 (d, J = 8.6 Hz, 2H), 6.43 (d, J = 4.1 Hz, 2H), 6.31 (d, J = 7.8 |
| | | Hz, 1H), 4.40 (s, 2H), 4.14-4.00 (m, 1H), 3.36 (dd, J = 9.2, 5.1 Hz, 1H), |
| | | 3.23 (s, 3H), 3.19 (dd, J = 9.2, 6.2 Hz, 1H), 1.37 (d, J = 2.0 Hz, 6H), 1.29 |
| | | (s, 9H), 1.09 (d, J = 6.6 Hz, 3H). |
| 269 | 439.4 | 1H NMR (400 MHz, DMSO) δ 7.88 (s, 1H), 7.49 (d, J = 8.4 Hz, 2H), 7.32 |
| | | (d, J = 8.3 Hz, 2H), 6.44 (s, 2H), 6.31 (d, J = 6.7 Hz, 1H), 4.40 (s, 2H), |
| | | 4.09 (d, J = 5.7 Hz, 1H), 3.38-3.35 (m, 1H), 3.25-3.15 (m, 4H), 1.37 (s, |
| | | 6H), 1.29 (s, 9H), 1.09 (d, J = 6.5 Hz, 3H). |
| 270 | 457.3 | 1H NMR (400 MHz, acetone) δ 7.99 (d, J = 5.1 Hz, 1H), 7.57-7.46 (m, |
| | | 2H), 7.46-7.33 (m, 2H), 6.71-6.63 (m, 1H), 6.59 (s, 1H), 6.23 (d, J = |
| | | 5.6 Hz, 1H), 4.54 (s, 2H), 4.34-4.18 (m, 1H), 3.01-2.96 (m, 2H), 2.61- |
| | | 2.39 (m, 2H), 1.44 (s, 6H), 1.35 (s, 9H). |
| 271 | 437.4 | 1H NMR (400 MHz, DMSO) δ 7.89 (d, J = 5.0 Hz, 1H), 7.53-7.45 (m, |
| | | 2H), 7.33-7.25 (m, 2H), 6.77 (s, 1H), 6.55-6.45 (m, 2H), 4.44 (s, 2H), |
| | | 3.45 (s, 2H), 1.37 (s, 6H), 1.29 (s, 9H), 0.77-0.74 (m, 2H), 0.58-0.56 (m, 2H). |
| 272 | 566.1 | 1H NMR (400 MHz, DMSO) δ (ppm): 8.67 (s, 1H), 8.10 (d, J = 3.1 Hz, |
| | | 1H), 8.09 (s, 1H), 7.86 (d, J = 8.5 Hz, 2H), 7.71-7.61 (m, 2H), 7.38 (t, J = |
| | | 7.7 Hz, 1H), 7.26 (d, J = 6.7 Hz, 1H), 6.91 (s, 1H), 6.84 (dd, J = 5.3, 1.2 |
| | | Hz, 1H), 4.55 (s, 2H), 4.41 (s, 2H), 3.05 (s, 3H), 1.43 (s, 6H). |
| 273 | 421.4 | 1H NMR (400 MHz, DMSO) δ 7.91 (s, 1H), 7.53-7.45 (m, 2H), 7.33- |
| | | 7.25 (m, 2H), 6.76 (s, 1H), 6.51 (d, J = 5.2 Hz, 2H), 4.46 (s, 2H), 1.38 (s, |
| | | 6H), 1.29 (d, J = 3.9 Hz, 12H), 0.70-0.54 (m, 4H). |
| 274 | 453.4 | 1H NMR (400 MHz, DMSO) δ 7.85 (d, J = 5.3 Hz, 1H), 7.50 (d, J = 8.7 |
| | | Hz, 2H), 7.32 (d, J = 8.6 Hz, 2H), 6.47 (s, 1H), 6.40 (d, J = 5.3 Hz, 1H), |
| | | 6.03 (s, 1H), 4.38 (s, 2H), 3.52 (s, 2H), 3.21 (s, 3H), 1.38 (s, 6H), 1.30 (s, |
| | | 6H), 1.29 (s, 9H). |
| 275 | 423.3 | 1H NMR (400 MHz, DMSO) δ 7.88 (d, J = 5.2 Hz, 1H), 7.50 (d, J = 8.6 |
| | | Hz, 2H), 7.32 (d, J = 8.6 Hz, 2H), 7.23 (d, J = 5.8 Hz, 1H), 6.53 (d, J = 4.6 |
| | | Hz, 1H), 6.45 (s, 1H), 4.87-4.82 (m, 1H), 4.76 (t, J = 6.5 Hz, 2H), 4.44 (s, |
| | | 2H), 4.39 (t, J = 6.1 Hz, 2H), 1.37 (s, 6H), 1.29 (s, 9H). |
| 276 | 433.3 | 1H NMR (400 MHz, DMSO) δ 7.91 (s, 1H), 7.55-7.41 (m, 2H), 7.38- |
| | | 7.23 (m, 2H), 7.13 (s, 1H), 6.52 (d, J = 4.8 Hz, 1H), 6.46 (s, 1H), 4.44 (s, |
| | | 2H), 2.24 (s, 1H), 2.02 (s, 6H), 1.36 (s, 6H), 1.29 (s, 9H). |
| 277 | 410.4 | 1H NMR (400 MHz, DMSO) δ (ppm): 8.64 (dd, J = 2.6, 0.6 Hz, 1H), 8.04 |
| | | (dd, J = 8.3, 2.6 Hz, 1H), 7.91 (d, J = 5.2 Hz, 1H), 7.43 (dd, J = 8.3, 0.6 |
| | | Hz, 1H), 6.44 (dd, J = 5.3, 1.4 Hz, 1H), 6.41 (s, 1H), 6.32 (d, J = 7.6 Hz, |
| | | 1H), 4.43 (s, 2H), 4.02-3.94 (m, 1H), 1.41 (s, 7H), 1.37 (s, 9H), 1.13 (d, |
| | | J = 6.4 Hz, 6H). |
| 278 | 434.3 | ¹H NMR (400 MHz, DMSO) δ (ppm): 7.92 (d, J = 5.2 Hz, 1H), 7.62- |
| | | 7.41 (m, 2H), 7.39-7.19 (m, 2H), 6.71 (d, J = 6.9 Hz, 1H), 6.57-6.40 |
| | | (m, 2H), 4.43 (s, 2H), 4.16-4.10 (m, 1H), 2.86-2.68 (m, 2H), 1.37 (d, J = |
| | | 2.9 Hz, 6H), 1.29 (s, 9H), 1.24 (d, J = 6.7 Hz, 3H). |
| 279 | 460.5 | 1H NMR (500 MHz, DMSO): δ 7.94 (d, J = 5.2 Hz, 1H), 7.90 (d, J = 2.2 |
| | | Hz, 1H), 7.76 (dd, J = 8.5, 2.2 Hz, 1H), 7.69 (d, J = 8.5 Hz, 1H), 6.75 (s, |
| | | 1H), 6.61 (d, J = 5.3 Hz, 1H), 6.53 (s, 1H), 4.50 (s, 2H), 3.87 (d, J = 8.3 |
| | | Hz, 2H), 3.63 (d, J = 8.2 Hz, 2H), 3.30-3.24 (m, 1H), 2.28-2.24 (m, |
| | | 1H), 1.76 (s, 2H), 1.40 (s, 6H), 1.30 (d, J = 6.9 Hz, 6H). |
| 280 | 522.3 | 1H NMR (500 MHz, DMSO) δ 7.95 (d, J = 5.2 Hz, 1H), 7.29 (d, J = 8.1 |
| | | Hz, 1H), 7.00 (d, J = 1.9 Hz, 1H), 6.94-6.91 (m, 1H), 6.74 (d, J = 2.2 Hz, |
| | | 1H), 6.59 (dd, J = 5.2, 1.1 Hz, 1H), 6.52 (s, 1H), 4.49 (s, 2H), 4.05 (t, J = |
| | | 5.7 Hz, 2H), 3.87 (d, J = 8.4 Hz, 2H), 3.64 (d, J = 8.2 Hz, 2H), 3.28 (dt, J = |
| | | 13.7, 7.0 Hz, 1H), 2.69 (t, J = 5.6 Hz, 2H), 2.25 (s, 7H), 1.76 (s, 2H), |
| | | 1.40 (s, 6H), 1.19 (d, J = 6.9 Hz, 6H). |
| 281 | 577.1 | 1H NMR (500 MHz, MeOD) δ 8.44 (d, J = 2.5 Hz, 1H), 7.93 (d, J = 5.6 |
| | | Hz, 1H), 7.51 (dd, J = 8.8, 1.4 Hz, 1H), 7.34 (dd, J = 8.8, 2.5 Hz, 1H), |
| | | 6.69 (m, 2H), 4.58 (s, 2H), 4.56 (s, 1H), 4.00 (d, J = 8.4 Hz, 2H), 3.73 (d, |
| | | J = 8.3 Hz, 2H), 3.18 (s, 2H), 2.42 (s, 6H), 2.35 (t, J = 2.2 Hz, 1H), 1.82 |
| | | (s, 2H), 1.47 (s, 6H). |
| 282 | 564.6 | 1H NMR (500 MHz, DMSO) δ 7.95 (d, J = 5.2 Hz, 1H), 7.50 (dd, J = 8.6, |
| | | 1.2 Hz, 1H), 7.37 (d, J = 2.3 Hz, 1H), 7.11 (dd, J = 8.6, 2.3 Hz, 1H), 6.73 |
| | | (d, J = 2.3 Hz, 1H), 6.61 (dd, J = 5.2, 1.4 Hz, 1H), 6.54 (s, 1H), 4.50 (s, |
| | | 2H), 4.14 (t, J = 5.7 Hz, 2H), 3.87 (d, J = 8.4 Hz, 2H), 3.64 (d, J = 8.2 Hz, |
| | | 2H), 2.67 (t, J = 5.7 Hz, 2H), 2.29-2.21 (m, 7H), 1.82-1.72 (m, 2H), |
| | | 1.41 (s, 6H). |
| 283 | 528.3 | 1H NMR (500 MHz, DMSO) δ 11.00 (s, 1H), 9.92 (s, 1H), 8.03 (d, J = |
| | | 5.5 Hz, 1H), 7.68 (d, J = 2.3 Hz, 1H), 7.56 (d, J = 8.7 Hz, 1H), 7.41 (d, J = |
| | | 8.3 Hz, 1H), 7.00 (d, J = 5.5 Hz, 1H), 4.54 (s, 2H), 3.66 (s, 2H), 3.08 (s, |
| | | 3H), 1.41 (s, 6H). |
| 284 | 522.4 | 1H NMR (500 MHz, DMSO) δ 8.32 (br, 2H), 8.03 (d, J = 5.5 Hz, 1H), |
| | | 7.50 (d, J = 7.5 Hz, 1H), 7.41 (d, J = 2.3 Hz, 1H), 7.14 (dd, J = 8.6, 2.3 |
| | | Hz, 1H), 6.99 (d, J = 5.5 Hz, 1H), 4.54 (s, 2H), 4.15 (t, J = 5.7 Hz, 2H), |
| | | 3.66 (s, 2H), 2.68 (t, J = 5.7 Hz, 2H), 2.24 (s, 6H), 1.41 (s, 6H). |
| 285 | 435.27 | 1H NMR(500 MHz, DMSO) δ 11.00 (s, 1H), 8.02 (d, J = 5.5 Hz, 1H), |
| | | 7.66-7.58 (m, 2H), 7.52 (d, J = 8.7 Hz, 2H), 6.99 (d, J = 5.5 Hz, 1H), |
| | | 4.53 (s, 2H), 3.65 (s, 2H), 1.40 (s, 6H). |
| 286 | 452.8 | 1H NMR (500 MHz, DMSO) δ 10.99 (s, 1H), 8.02 (d, J = 5.5 Hz, 1H), |
| | | 7.79-7.71 (m, 2H), 7.55-7.49 (m, 1H), 7.01 (d, J = 5.5 Hz, 1H), 4.54 (s, |
| | | 2H), 3.65 (s, 2H), 1.40 (s, 6H). |
| 287 | 468.9 | 1H NMR(500 MHz, DMSO) δ 11.00 (s, 1H), 8.02 (d, J = 5.5 Hz, 1H), |
| | | 7.90 (d, J = 2.4 Hz, 1H), 7.75 (dd, J = 8.8, 1.1 Hz, 1H), 7.64 (dd, J = 8.9, |
| | | 2.4 Hz, 1H), 7.02 (d, J = 5.5 Hz, 1H), 4.53 (s, 2H), 3.65 (s, 2H), 1.40 (s, 6H). |
| 288 | 460.2 | 1H NMR(500 MHz, DMSO) δ 10.94 (s, 1H), 8.23 (d, J = 2.5 Hz, 1H), |
| | | 8.05-8.01 (m, 2H), 7.89 (dd, J = 9.0, 1.4 Hz, 1H), 7.02 (d, J = 5.5 Hz, |
| | | 1H), 4.54 (s, 2H), 3.66 (s, 2H), 1.41 (s, 6H) |
| 289 | 473.1 | 1H NMR (400 MHz, DMSO) δ 7.85 (d, J = 5.2 Hz, 1H), 7.77 (d, J = 8.5 |
| | | Hz, 2H), 7.30-7.19 (m, 5H), 7.17-7.10 (m, 2H), 6.44 (dd, J = 5.2, 1.2 |
| | | Hz, 1H), 6.35 (s, 1H), 5.91 (s, 2H), 4.69 (d, J = 16.3 Hz, 1H), 4.50 (t, J = |
| | | 4.3 Hz, 1H), 4.35 (d, J = 16.3 Hz, 1H), 3.31-3.26 (m, 1H), 3.12 (dd, J = |
| | | 14.3, 3.8 Hz, 1H). some signal is under the w“ter peak |
| 290 | 425.5 | ″1H NMR (400 MHz, DMSO) δ 7.89-7.81 (m, 3H), 7.62-7.55 (m, 2H), |
| | | 6.45 (dd, J = 5.2, 1.1 Hz, 1H), 6.37 (s, 1H), 5.92 (s, 2H), 4.51 (d, J = 16.4 |
| | | Hz, 1H), 4.35 (d, J = 16.4 Hz, 1H), 4.10 (d, J = 3.1 Hz, 1H), 2.27-2.08 |
| | | (m, 1H), 1.06 (d, J = 7.0 Hz, 3H), 0.81 (d, J = 6.8 Hz, 3H). |
| 291 | 437.2 | 1H NMR (400 MHz, DMSO) δ (ppm): 7.85-7.81 (m, 3H), 7.65 (d, J = 8.6 |
| | | Hz, 2H), 6.45 (dd, J = 5.3, 1.1 Hz, 1H), 6.37 (s, 1H), 5.89 (s, 2H), 4.42 (s, |
| | | 2H), 2.05-1.99 (m, 2H), 1.95-1.81 (m, 2H), 1.75-1.70 (m, 4H). |
| 292 | 423.3 | 1H NMR (400 MHz, DMSO) δ 7.84 (t, J = 6.6 Hz, 3H), 7.64 (d, J = 8.5 |
| | | Hz, 2H), 6.46 (d, J = 4.7 Hz, 1H), 6.35 (s, 1H), 5.89 (s, 2H), 4.61 (s, 2H), |
| | | 2.58-2.49 (m, 4H), 2.40-2.33 (m, 2H), 2.02-1.94 (m, 1H), 1.76-1.70 (m, 1H). |
| 293 | 451.3 | 1H NMR (400 MHz, DMSO) δ 7.84 (d, J = 8.5 Hz, 2H), 7.80 (d, J = 5.2 |
| | | Hz, 1H), 7.63 (d, J = 8.6 Hz, 2H), 6.45 (d, J = 5.2 Hz, 1H), 6.38 (s, 1H), |
| | | 5.85 (s, 2H), 4.43 (s, 2H), 1.90-1.83 (m, 4H), 1.75-1.53 (m, 5H), 1.27- |
| | | 1.03 (m, 1H). |
| 294 | 459.2 | 1H NMR (400 MHz, DMSO) δ 9.42 (br s, 1H), 7.89-7.55 (m, 6H), 7.51- |
| | | 7.37 (m, 3H), 7.24 (d, J = 8.1 Hz, 2H), 6.45-6.26 (m, 2H), 5.93 (s, 2H), |
| | | 3.50 (d, J = 13.1 Hz, 1H), 2.97 (d, J = 13.2 Hz, 1H). |
| 295 | 425.2 | 1H NMR (400 MHz, DMSO) δ 7.92-7.77 (m, 3H), 7.58 (d, J = 8.6 Hz, |
| | | 2H), 6.46 (dd, J = 5.2, 1.2 Hz, 1H), 6.37 (s, 1H), 5.93 (s, 2H), 4.51 (d, J = |
| | | 16.4 Hz, 1H), 4.36 (d, J = 16.4 Hz, 1H), 4.11 (d, J = 3.1 Hz, 1H), 2.26- |
| | | 2.10 (m, 1H), 1.06 (d, J = 7.0 Hz, 3H), 0.81 (d, J = 6.8 Hz, 3H). |
| 296 | 465.3 | 1H NMR (400 MHz, DMSO) δ 7.90-7.79 (m, 3H), 7.57 (d, J = 8.6 Hz, |
| | | 2H), 6.46 (dd, J = 5.2, 1.1 Hz, 1H), 6.38 (s, 1H), 5.92 (s, 2H), 4.50 (d, J = |
| | | 16.3 Hz, 1H), 4.38 (d, J = 16.3 Hz, 1H), 4.07 (d, J = 2.9 Hz, 1H), 1.91- |
| | | 1.80 (m, 1H), 1.74-1.50 (m, 6H), 1.24-0.81 (m, 4H). |
| 297 | 425.2 | 1H NMR (400 MHz, DMSO) δ (ppm): 7.84 (dd, J = 10.6, 7.0 Hz, 3H), |
| | | 7.67-7.56 (m, 2H), 6.52 (dd, J = 5.3, 1.4 Hz, 1H), 6.45 (s, 1H), 5.87 (s, |
| | | 2H), 4.47 (d, J = 16.5 Hz, 1H), 4.32 (d, J = 16.5 Hz, 1H), 1.93-1.68 (m, |
| | | 2H), 1.37 (s, 3H), 0.68 (t, J = 7.3 Hz, 3H). |
| 298 | 455.4 | 1H NMR (400 MHz, DMSO) δ 7.91 (d, J = 5.2 Hz, 1H), 7.87 (d, J = 8.5 |
| | | Hz, 2H), 7.63-7.58 (m, 2H), 6.45 (dd, J = 5.3, 1.3 Hz, 1H), 6.37 (s, 1H), |
| | | 6.27 (d, J = 7.6 Hz, 1H), 5.31 (t, J = 5.3 Hz, 1H), 4.47 (dd, J = 215.9, 16.5 |
| | | Hz, 2H), 4.20 (t, J = 2.4 Hz, 1H), 3.98 (dd, J = 13.8, 6.5 Hz, 1H), 3.90- |
| | | 3.77 (m, 2H), 1.12 (d, J = 6.4 Hz, 6H). |
| 299 | 469.3 | 1H NMR (400 MHz, DMSO-D6) δ 7.91-7.84 (m, 3H), 7.64-7.58 (m, |
| | | 2H), 6.47 (dd, J = 5.3, 1.3 Hz, 1H), 6.43 (s, 1H), 6.25 (d, J = 7.6 Hz, 1H), |
| | | 5.49 (t, J = 5.4 Hz, 1H), 4.59 (d, J = 16.9 Hz, 1H), 4.24 (d, J = 16.9 Hz, |
| | | 1H), 4.03-3.90 (m, 1H), 3.74 (dd, J = 11.6, 5.0 Hz, 1H), 3.63 (dd, J = |
| | | 11.6, 5.8 Hz, 1H), 1.22 (s, 3H), 1.11 (d, J = 6.4 Hz, 6H). |
| 300 | 457.4 | 1H NMR (400 MHz, DMSO) δ 8.29 (d, J = 8.7 Hz, 2H), 7.96 (d, J = 8.8 |
| | | Hz, 2H), 7.84 (d, J = 5.2 Hz, 1H), 6.48 (d, J = 5.2 Hz, 1H), 6.39 (s, 1H), |
| | | 5.91 (s, 2H), 4.53 (d, J = 16.4 Hz, 1H), 4.38 (d, J = 16.4 Hz, 1H), 4.14 (d, |
| | | J = 3.1 Hz, 1H), 2.28-2.12 (m, 1H), 1.07 (d, J = 7.0 Hz, 3H), 0.81 (d, J = |
| | | 6.8 Hz, 3H). |
| 301 | 505.3 | ¹H NMR (400 MHz, DMSO) δ (ppm): 7.91 (d, J = 5.3 Hz, 1H), 7.85 (d, J = |
| | | 8.5 Hz, 2H), 7.71-7.60 (m, 2H), 7.54 (s, 1H), 7.31 (s, 1H), 6.62 (s, |
| | | 1H), 6.49 (d, J = 5.3 Hz, 1H), 6.45 (s, 1H), 4.43 (s, 2H), 4.22 (d, J = 5.5 |
| | | Hz, 2H), 3.73 (s, 3H), 1.37 (s, 6H). |
| 302 | 534.4 | 1H NMR (400 MHz, DMSO) δ (ppm): 10.42 (s, 1H), 8.35 (s, 1H), 8.22 |
| | | (d, J = 5.1 Hz, 1H), 8.13 (s, 1H), 7.85 (d, J = 8.5 Hz, 2H), 7.64 (d, J = 8.6 |
| | | Hz, 2H), 7.12 (d, J = 4.2 Hz, 1H), 4.61 (s, 2H), 3.83 (d, J = 25.4 Hz, 4H), |
| | | 3.21-3.08 (m, 1H), 2.42-2.27 (m, 4H), 1.40 (s, 6H). |
| 303 | 524.2 | 1H NMR (400 MHz, DMSO) δ (ppm): 9.12 (s, 1H), 8.40 (d, J = 5.6 Hz, |
| | | 1H), 8.11 (d, J = 5.3 Hz, 1H), 7.86 (d, J = 8.5 Hz, 2H), 7.65 (d, J = 8.5 Hz, |
| | | 2H), 7.34 (s, 1H), 6.95 (d, J = 5.2 Hz, 1H), 4.57 (s, 2H), 4.33-4.17 (m, |
| | | 1H), 3.86-3.63 (m, 3H), 3.50 (dd, J = 8.9, 3.3 Hz, 1H), 2.21-2.08 (m, |
| | | 1H), 1.80-1.65 (m, 1H), 1.40 (s, 6H). |
| 304 | 548.2 | 1H NMR (400 MHz, DMSO) δ (ppm): 9.00 (s, 1H), 8.43 (s, 1H), 8.09 (d, |
| | | J = 5.3 Hz, 1H), 7.85 (d, J = 8.5 Hz, 2H), 7.73-7.61 (m, 2H), 7.35 (s, |
| | | 1H), 6.92 (dd, J = 5.3, 1.4 Hz, 1H), 4.56 (s, 2H), 2.14-2.07 (m, 1H), 1.82- |
| | | 1.52 (m, 8H), 1.40 (s, 6H), 1.37-1.28 (m, 2H). |
| 305 | 562.6 | 1H NMR (400 MHz, DMSO) δ 10.05 (s, 1H), 8.24 (s, 1H), 8.16 (s, 1H), |
| | | 7.85 (d, J = 8.5 Hz, 2H), 7.73-7.60 (m, 2H), 7.17 (dd, J = 5.2, 1.3 Hz, |
| | | 1H), 4.65 (s, 2H), 3.84 (s, 1H), 3.32 (d, J = 7.3 Hz, 1H), 3.05 (d, J = 7.3 |
| | | Hz, 1H), 1.80 (d, J = 13.3 Hz, 1H), 1.68-1.03 (m, 16H). One compound |
| | | signal is under the water peak |
| 306 | 538.2 | ¹H NMR (400 MHz, DMSO) δ (ppm): 9.10 (s, 1H), 8.27 (s, 1H), 8.11 (d, |
| | | J = 5.3 Hz, 1H), 7.85 (d, J = 8.5 Hz, 2H), 7.71-7.59 (m, 2H), 7.33 (s, |
| | | 1H), 6.94 (dd, J = 5.4, 1.3 Hz, 1H), 4.56 (s, 2H), 3.84-3.64 (m, 3H), 3.45- |
| | | 3.32 (m, 2H), 1.81 (d, J = 9.7 Hz, 2H), 1.48-1.30 (m, 8H). |
| 307 | 619.2 | ¹H NMR (400 MHz, DMSO) δ (ppm): 9.10 (s, 1H), 8.30 (s, 1H), 8.10 (d, |
| | | J = 5.3 Hz, 1H), 7.85 (d, J = 8.5 Hz, 2H), 7.75-7.58 (m, 2H), 7.31 (s, |
| | | 1H), 6.94 (dd, J = 5.3, 1.4 Hz, 1H), 4.56 (s, 2H), 3.55 (d, J = 7.8 Hz, 1H), |
| | | 3.15 (q, J = 10.2 Hz, 2H), 2.82 (d, J = 12.2 Hz, 2H), 2.44 (d, J = 9.6 Hz, |
| | | 2H), 1.80 (d, J = 9.7 Hz, 2H), 1.49-1.32 (m, 8H). |
| 308 | 510.5 | 1H NMR (400 MHz, DMSO) δ (ppm): 9.26 (s, 1H), 8.84 (d, J = 6.3 Hz, |
| | | 1H), 8.16 (d, J = 5.3 Hz, 1H), 7.86 (d, J = 8.5 Hz, 2H), 7.72-7.60 (m, |
| | | 2H), 7.33 (s, 1H), 6.98 (dd, J = 5.3, 1.3 Hz, 1H), 4.86-4.77 (m, 1H), 4.74 |
| | | (t, J = 6.6 Hz, 2H), 4.58 (s, 2H), 4.45 (t, J = 6.2 Hz, 2H), 1.40 (s, 6H). |
| 309 | 551.2 | 1H NMR (400 MHz, DMSO) δ 9.09 (s, 1H), 8.26 (s, 2H), 8.11 (d, J = 5.3 |
| | | Hz, 1H), 7.86 (d, J = 8.5 Hz, 2H), 7.71-7.60 (m, 2H), 7.33 (s, 1H), 6.94 |
| | | (dd, J = 5.3, 1.2 Hz, 1H), 4.57 (s, 2H), 3.55-3.49 (m, 1H), 2.64-2.58 (m, |
| | | 2H), 2.16 (s, 3H), 2.06 (t, J = 10.5 Hz, 2H), 1.83-1.80 (m, 2H), 1.47-1.35 |
| | | (m, 8H)* formate salt. |
| 310 | 495.4 | 1H NMR (400 MHz, DMSO) δ (ppm): 7.88-7.84 (m, 3H), 7.69-7.56 (m, |
| | | 2H), 6.55-6.33 (m, 3H), 4.42 (s, 2H), 3.93-3.73 (m, 3H), 3.41-3.34 |
| | | (m, 2H), 1.85-1.82 (m, 2H), 1.45-1.26 (m, 8H). |
| 311 | 465.3 | ¹H NMR (400 MHz, DMSO) δ (ppm): 7.87-7.84 (m, 3H), 7.69-7.61 (m, |
| | | 2H), 6.50 (t, J = 5.5 Hz, 1H), 6.49-6.45 (m, 2H), 4.43 (s, 2H), 3.11-3.01 |
| | | (m, 2H), 1.39 (s, 6H), 1.03-0.96 (m, 1H), 0.50-0.34 (m, 2H), 0.25-0.12 |
| | | (m, 2H). |
| 312 | 491.1 | 1H NMR (400 MHz, DMSO) δ (ppm): 11.75 (s, 1H), 7.92 (d, J = 5.2 Hz, |
| | | 1H), 7.85 (d, J = 8.5 Hz, 2H), 7.67-7.63 (m, 2H), 6.96 (s, 1H), 6.81 (t, J = |
| | | 5.5 Hz, 1H), 6.75 (s, 1H), 6.57-6.51 (m, 2H), 4.44 (s, 2H), 4.40 (d, J = |
| | | 5.5 Hz, 2H), 1.37 (s, 6H). |
| 313 | 493.2 | ¹H NMR (400 MHz, DMSO) δ (ppm): 7.95 (d, J = 5.3 Hz, 1H), 7.85 (d, J = |
| | | 8.5 Hz, 2H), 7.68-7.62 (m, 2H), 7.07 (t, J = 6.6 Hz, 1H), 6.65-6.61 |
| | | (m, 1H), 6.60 (s, 1H), 4.47 (s, 2H), 4.18-4.07 (m, 2H), 1.40 (s, 6H). |
| 314 | 467.6 | 1H NMR (400 MHz, DMSO) δ 8.42 (dd, J = 7.0, 0.6 Hz, 1H), 7.91-7.84 |
| | | (m, 2H), 7.71 (s, 1H), 7.67 (d, J = 8.8 Hz, 2H), 7.46 (s, 1H), 6.82 (dd, J = |
| | | 7.0, 1.6 Hz, 1H), 6.57 (d, J = 7.7 Hz, 1H), 5.25 (d, J = 7.5 Hz, 1H), 5.11 (t, |
| | | J = 5.7 Hz, 1H), 4.54 (d, J = 5.4 Hz, 2H), 4.49-4.43 (m, 2H), 1.17 (s, 3H), |
| | | 1.14 (s, 3H). |
| 315 | 475.3 | ¹H NMR (400 MHz, DMSO) δ (ppm): 7.92 (d, J = 5.2 Hz, 1H), 7.85 (d, J = |
| | | 8.5 Hz, 2H), 7.75-7.54 (m, 2H), 6.87 (t, J = 6.1 Hz, 1H), 6.69-6.45 |
| | | (m, 2H), 6.06 (tt, J = 56.6, 4.1 Hz, 1H), 4.46 (s, 2H), 3.78-3.55 (m, 2H), |
| | | 1.39 (s, 6H). |
| 316 | 527.2 | ¹H NMR (400 MHz, DMSO) δ (ppm): 8.30 (d, J = 8.8 Hz, 2H), 8.14- |
| | | 7.94 (m, 2H), 7.88 (d, J = 5.3 Hz, 1H), 6.48 (dd, J = 5.3, 1.4 Hz, 1H), 6.44 |
| | | (s, 1H), 6.39 (d, J = 7.5 Hz, 1H), 4.44 (s, 2H), 3.97-3.76 (m, 3H), 3.39- |
| | | 3.31 (m, 2H), 1.87-1.77 (m, 2H), 1.41 (s, 6H), 1.40-1.31 (m, 2H). |
| 317 | 507.3 | 1H NMR (400 MHz, DMSO) δ (ppm): 8.34-8.26 (m, 2H), 8.08-7.99 |
| | | (m, 2H), 7.94 (d, J = 5.3 Hz, 1H), 6.87 (br. t, J = 6.1 Hz, 1H), 6.62 (dd, J = |
| | | 5.3, 1.4 Hz, 1H), 6.59 (s, 1H), 6.08 (tt, J = 56.5, 4.1 Hz, 1H), 4.49 (s, 2H), |
| | | 3.74-3.60 (m, 2H), 1.43 (s, 6H). |
| 318 | 515.3 | 1H NMR (400 MHz, DMSO) δ (ppm): 8.34-8.28 (m, 2H), 8.08-8.00 |
| | | (m, 2H), 7.89 (d, J = 5.1 Hz, 1H), 6.52-6.44 (m, 2H), 6.26 (d, J = 7.9 Hz, |
| | | 1H), 4.46 (s, 2H), 4.15-4.02 (m, 1H), 3.37 (dd, J = 9.2, 5.1 Hz, 1H), 3.24 |
| | | (s, 3H), 3.21 (dd, J = 9.2, 6.2 Hz, 1H), 1.43 (s, 3H), 1.43 (s, 3H), 1.10 (d, J = |
| | | 6.6 Hz, 3H). |
| 319 | 525.2 | 1H NMR (400 MHz, DMSO) δ (ppm): 8.34-8.28 (m, 2H), 8.06-8.01 |
| | | (m, 2H), 7.94 (d, J = 5.2 Hz, 1H), 6.73 (d, J = 2.3 Hz, 1H), 6.63 (dd, J = |
| | | 5.2, 1.2 Hz, 1H), 6.53 (s, 1H), 4.52 (s, 2H), 3.86 (d, J = 8.4 Hz, 2H), 3.65- |
| | | 3.58 (d, J = 8.2 Hz, 2H), 2.29-2.23 (m, 1H), 1.78-1.70 (m, 2H), 1.43 |
| | | (s, 6H). |
| 320 | 513.2 | 1H NMR (400 MHz, DMSO) δ (ppm): 8.36-8.27 (m, 2H), 8.07-8.00 |
| | | (m, 2H), 7.92 (d, J = 5.3 Hz, 1H), 6.68 (d, J = 6.2 Hz, 1H), 6.54 (dd, J = |
| | | 5.3, 1.3 Hz, 1H), 6.49 (s, 1H), 4.47 (s, 2H), 4.39-4.27 (m, 1H), 3.88- |
| | | 3.77 (m, 2H), 3.69 (td, J = 8.1, 5.6 Hz, 1H), 3.48 (dd, J = 8.7, 3.9 Hz, 1H), |
| | | 2.14 (dq, J = 12.6, 7.5 Hz, 1H), 1.81-1.70 (m, 1H), 1.43 (s, 6H). |
| 321 | 510.2 | 1H NMR (400 MHz, DMSO) δ (ppm): 8.34-8.29 (m, 2H), 8.07-8.01 |
| | | (m, 2H), 7.94 (d, J = 5.3 Hz, 1H), 6.68 (d, J = 7.0 Hz, 1H), 6.58 (dd, J = |
| | | 5.3, 1.4 Hz, 1H), 6.53 (s, 1H), 4.48 (s, 2H), 4.20-4.08 (m, 1H), 2.85 (dd, |
| | | J = 16.7, 5.8 Hz, 1H), 2.72 (dd, J = 16.7, 4.9 Hz, 1H), 1.44 (s, 3H), 1.43 |
| | | (s, 3H), 1.25 (d, J = 6.7 Hz, 3H). |
| 322 | 604.1 | 1H NMR (400 MHz, DMSO) δ (ppm): 8.34-8.29 (m, 2H), 8.06-8.02 |
| | | (m, 2H), 7.91 (d, J = 5.3 Hz, 1H), 6.52 (dd, J = 5.3, 1.4 Hz, 1H), 6.49 (d, J = |
| | | 7.4 Hz, 1H), 6.47 (s, 1H), 4.47 (s, 2H), 3.88-3.77 (m, 1H), 3.50 (dt, J = |
| | | 11.8, 3.3 Hz, 2H), 2.91-2.81 (m, 5H), 1.98 (dd, J = 13.1, 3.3 Hz, 2H), |
| | | 1.43 (s, 8H). |
| 323 | 540.1 | 1H NMR (400 MHz, DMSO) δ 8.30-8.24 (m, 2H), 8.02-7.95 (m, 2H), |
| | | 7.89 (d, J = 5.3 Hz, 1H), 6.83 (d, J = 6.0 Hz, 1H), 6.54 (dd, J = 5.3, 1.4 |
| | | Hz, 1H), 6.44 (s, 1H), 4.44 (s, 2H), 4.38-4.29 (m, 1H), 3.64 (dd, J = |
| | | 10.1, 6.7 Hz, 1H), 3.11 (dd, J = 10.1, 3.7 Hz, 1H), 2.67 (s, 3H), 2.60 (dd, J = |
| | | 16.8, 7.8 Hz, 1H), 2.11 (dd, J = 16.8, 4.4 Hz, 1H), 1.39 (s, 6H). |
| 324 | 540.3 | 1H NMR (400 MHz, DMSO) δ 8.32 (d, J = 8.8 Hz, 2H), 8.06-7.98 (m, |
| | | 2H), 7.93 (d, J = 5.2 Hz, 1H), 6.87 (d, J = 6.0 Hz, 1H), 6.58 (dd, J = 5.3, |
| | | 1.4 Hz, 1H), 6.48 (s, 1H), 4.48 (s, 2H), 4.42-4.33 (m, 1H), 3.68 (dd, J = |
| | | 10.1, 6.7 Hz, 1H), 3.16 (dd, J = 10.1, 3.6 Hz, 1H), 2.71 (s, 3H), 2.64 (dd, J = |
| | | 16.6, 7.9 Hz, 1H), 2.15 (dd, J = 16.7, 4.3 Hz, 1H), 1.43 (s, 6H). |
| 325 | 563.4 | 1H NMR (400 MHz, DMSO) δ 12.20 (s, 1H), 8.27 (d, J = 8.8 Hz, 2H), |
| | | 8.03-7.96 (m, 2H), 7.88 (d, J = 6.0 Hz, 1H), 7.24 (s, 1H), 6.46 (dd, J = |
| | | 13.3, 6.1 Hz, 3H), 4.43 (s, 2H), 4.14-4.01 (m, 1H), 2.93 (dd, J = 15.6, |
| | | 5.1 Hz, 1H), 2.59-2.52 (m, 1H), 1.99-1.89 (m, 1H), 1.66-1.52 (m, |
| | | 1H), 1.39 (s, 6H). Two protons overlap with the solvent signals. |
| 326 | 554.3 | 1H NMR(400 MHz, DMSO) δ 8.31 (d, J = 8.8 Hz, 2H), 8.04 (d, J = 8.8 |
| | | Hz, 2H), 7.92 (d, J = 5.3 Hz, 1H), 6.56 (s, 1H), 6.54 (s, 1H), 6.49 (s, 1H), |
| | | 4.47 (s, 2H), 4.17-4.06 (m, 1H), 3.32-3.23 (m, 2H), 2.81 (s, 3H), 2.57 |
| | | (dd, J = 16.9, 5.0 Hz, 1H), 2.17 (dd, J = 17.0, 8.5 Hz, 1H), 2.13-2.00 (m, |
| | | 1H), 1.79-1.65 (m, 1H), 1.43 (s, 6H). |
| 327 | 547.3 | 1H NMR (500 MHz, DMSO) δ 8.35-8.27 (m, 2H), 8.08-8.02 (m, 2H), |
| | | 7.99 (d, J = 5.4 Hz, 1H), 7.27 (d, J = 5.1 Hz, 1H), 6.69 (d, J = 5.3 Hz, 1H), |
| | | 6.58 (s, 1H), 4.60-4.50 (m, 4H), 4.50-4.42 (m, 1H), 4.06-3.95 (m, |
| | | 2H), 1.44 (s, 6H). |
| 328 | 554.4 | 1H NMR(400 MHz, DMSO) δ 8.30 (d, J = 8.8 Hz, 2H), 8.03 (d, J = 8.8 |
| | | Hz, 2H), 7.87 (d, J = 5.3 Hz, 1H), 6.58 (s, 1H), 6.56 (d, J = 6.8 Hz, 1H), |
| | | 6.52 (d, J = 5.3 Hz, 1H), 4.46 (s, 2H), 4.36 (dt, J = 10.8, 6.0 Hz, 1H), 3.30- |
| | | 3.25 (m, 3H), 2.81 (s, 3H), 2.23-2.13 (m, 1H), 1.90-1.77 (m, 1H), |
| | | 1.68-1.53 (m, 1H), 1.42 (s, 6H) |
| 329 | 590.3 | 1H NMR (400 MHz, DMSO) δ 8.35-8.26 (m, 2H), 8.07-8.01 (m, 2H), |
| | | 7.95 (d, J = 5.3 Hz, 1H), 6.75 (d, J = 6.0 Hz, 1H), 6.59 (dd, J = 5.3, 1.3 |
| | | Hz, 1H), 6.51 (s, 1H), 4.49 (s, 2H), 4.39-4.30 (m, 1H), 3.53 (dd, J = |
| | | 10.2, 6.0 Hz, 1H), 3.45-3.37 (m, 1H), 3.33-3.28 (m, 1H), 3.08 (dd, J = |
| | | 10.2, 4.5 Hz, 1H), 2.86 (s, 3H), 2.24-2.12 (m, 1H), 1.91-1.80 (m, 1H), |
| | | 1.43 (s, 6H). |
| 330 | 554.3 | 1H NMR(400 MHz, DMSO) δ 8.32 (d, J = 8.8 Hz, 2H), 8.04 (d, J = 8.8 |
| | | Hz, 2H), 7.93 (d, J = 5.3 Hz, 1H), 6.64 (d, J = 7.1 Hz, 1H), 6.56 (d, J = 5.3 |
| | | Hz, 1H), 6.53 (s, 1H), 4.48 (s, 2H), 4.27-4.12 (m, 1H), 3.53 (dd, J = |
| | | 11.9, 4.8 Hz, 1H), 3.12 (dd, J = 11.9, 7.2 Hz, 1H), 2.78 (s, 3H), 2.30 (m, |
| | | 2H), 2.01-1.71 (m, 2H), 1.44 (s, 6H). |
| 331 | 510.3 | 1H NMR (400 MHz, DMSO) δ (ppm): 8.30 (d, J = 8.8 Hz, 2H), 8.06- |
| | | 7.99 (m, 2H), 7.92 (d, J = 5.3 Hz, 1H), 6.66 (d, J = 7.0 Hz, 1H), 6.56 (dd, |
| | | J = 5.3, 1.4 Hz, 1H), 6.52 (s, 1H), 4.47 (s, 2H), 4.18-4.08 (m, 1H), 2.83 |
| | | (dd, J = 16.7, 5.8 Hz, 1H), 2.71 (dd, J = 16.7, 4.9 Hz, 1H), 1.42 (s, J = 2.2 |
| | | Hz, 3H), 1.42 (s, 3H), 1.24 (d, J = 6.7 Hz, 3H). |
| 332 | 540.3 | 1H NMR (500 MHz, DMSO) δ 8.32 (d, J = 8.8 Hz, 2H), 8.07-8.03 (m, |
| | | 2H), 7.90 (d, J = 5.2 Hz, 1H), 6.67 (d, J = 7.2 Hz, 1H), 6.60 (s, 1H), 6.57 |
| | | (dd, J = 5.3, 1.4 Hz, 1H), 4.56-4.46 (m, 3H), 3.32-3.29 (m, overlaps |
| | | with solvent, 2H), 2.77 (s, 3H), 2.45 (m, 1H), 1.75 (m, 1H), 1.45 (two |
| | | singlets, J = 1.2 Hz, 6H). |
| 333 | 540.2 | 1H NMR(400 MHz, DMSO) δ 8.31 (d, J = 8.7 Hz, 2H), 8.04 (d, J = 8.8 |
| | | Hz, 2H), 7.92 (d, J = 5.3 Hz, 1H), 7.53 (s, 1H), 6.57-6.51 (m, 2H), 6.50 |
| | | (s, 1H), 4.47 (s, 2H), 4.16-4.01 (m, 1H), 3.24-3.07 (m, 2H), 2.11 (dd, J = |
| | | 17.1, 8.4 Hz, 1H), 2.04-1.91 (m, 1H), 1.72-1.52 (m, 1H), 1.44 (s, 6H). |
| 334 | 545.3 | 1H NMR (400 MHz, DMSO) δ (ppm): 8.32 (d, J = 8.8 Hz, 2H), 8.04 (d, J = |
| | | 8.8 Hz, 2H), 7.92 (d, J = 5.3 Hz, 1H), 6.70 (d, J = 7.5 Hz, 1H), 6.57 (d, J = |
| | | 5.4 Hz, 1H), 6.53 (s, 1H), 4.49 (s, 2H), 4.57-4.35 (m, 1H), 4.25-4.10 |
| | | (m, 1H), 3.93 (ddd, J = 15.6, 11.8, 3.7 Hz, 1H), 3.84-3.70 (m, 1H), 3.57- |
| | | 3.38 (m, 2H), 2.09-1.97 (m, 1H), 1.54-1.47 (m, 1H), 1.44 (s, 6H). |
| 335 | 525.3 | 1H NMR (400 MHz, DMSO) δ (ppm): 8.35-8.28 (m, 2H), 8.07-8.01 |
| | | (m, 2H), 7.92 (d, J = 5.2 Hz, 1H), 6.62 (s, 1H), 6.59 (dd, J = 5.2, 1.3 Hz, |
| | | 1H), 6.24 (d, J = 3.1 Hz, 1H), 4.50 (s, 2H), 3.84-3.77 (m, 2H), 3.76-3.66 |
| | | (m, 2H), 2.82 (td, J = 6.7, 3.1 Hz, 1H), 2.02-1.94 (m, 2H), 1.43 (s, 6H). |
| 336 | 503.2 | 1H NMR (400 MHz, DMSO) δ 8.30 (d, J = 8.8 Hz, 2H), 8.07-7.95 (m, |
| | | 2H), 7.89 (d, J = 5.3 Hz, 1H), 6.51 (dd, J = 9.3, 3.9 Hz, 2H), 6.45 (d, J = |
| | | 7.6 Hz, 1H), 4.48-4.11 (m, 5H), 1.43 (d, J = 6.8 Hz, 6H), 1.17-1.08 (m, 3H). |
| 337 | 471.2 | 1H NMR (400 MHz, DMSO) δ 8.07 (d, J = 8.8 Hz, 2H), 7.95 (d, J = 5.2 |
| | | Hz, 1H), 7.77 (d, J = 8.8 Hz, 2H), 6.73 (d, J = 2.3 Hz, 1H), 6.61 (dd, J = |
| | | 5.2, 1.3 Hz, 1H), 6.53 (s, 1H), 4.51 (s, 2H), 3.86 (d, J = 8.4 Hz, 2H), 3.63 |
| | | (d, J = 8.2 Hz, 2H), 3.27 (s, 3H), 2.26 (d, J = 2.2 Hz, 1H), 1.75 (s, 2H), |
| | | 1.42 (s, 6H). |
| 338 | 515.3 | 1H NMR (400 MHz, DMSO) δ 8.34-8.28 (m, 2H), 8.07-8.01 (m, 2H), |
| | | 7.89 (d, J = 5.2 Hz, 1H), 6.54 (s, 1H), 6.50 (dd, J = 5.3, 1.3 Hz, 1H), 6.39 |
| | | (t, J = 5.8 Hz, 1H), 4.46 (s, 2H), 3.50-3.41 (m, 1H), 3.30-3.26 (m, 2H), |
| | | 3.24 (s, 3H), 1.43 (s, 6H), 1.08 (d, J = 6.2 Hz, 3H). |
| 339 | 540.5 | 1H NMR (400 MHz, DMSO) δ 8.31-8.28 m, 2H), 8.13-7.95 (m, 2H), |
| | | 7.85 (d, J = 5.7 Hz, 1H), 6.53-6.37 (m, 2H), 6.23 (d, J = 8.8 Hz, 1H), |
| | | 4.43 (s, 2H), 4.01-3.95 (m, 1H), 3.77-3.65 (m, 2H), 3.65-3.49 (m, 1H), |
| | | 1.87-1.71 (m, 3H), 1.66-1.53 (m, 1H), 1.42/1.41 (2 s, 6H 1.05 (d, J = |
| | | 6.5 Hz, 3H). |
| 340 | 541.4 | 1H NMR (400 MHz, DMSO) δ 8.31-8.28 (m, 2H), 8.05-8.00 (m, 2H), |
| | | 7.85 (d, J = 5.3 Hz, 1H), 6.51 (s, 1H), 6.45 (dd, J = 5.3, 1.3 Hz, 1H), 6.10 |
| | | (d, J = 8.4 Hz, 1H), 4.43 (s, 2H), 4.11-4.06 (m, 1H), 3.83-3.70 (m, 2H), |
| | | 3.60-3.55 (m, 1H), 1.79-1.74 (m, 3H), 1.61-1.50 (m, 1H), 1.42/1.42 (2 s, |
| | | 6H), 1.06 (d, J = 6.7 Hz, 3H). |
| 341 | 541.4 | 1H NMR (400 MHz, DMSO) δ 8.31-8.28 (m, 2H), 8.08-7.99 (m, 2H), |
| | | 7.85 (d, J = 5.3 Hz, 1H), 6.50 (s, 1H), 6.45 (dd, J = 5.3, 1.3 Hz, 1H), 6.09 |
| | | (d, J = 8.4 Hz, 1H), 4.43 (s, 2H), 4.11-4.06 (m, 1H), 3.82-3.71 (m, 2H), |
| | | 3.58 (dd, J = 14.7, 6.7 Hz, 1H), 1.84-1.73 (m, 3H), 1.62-1.51 (m, 1H), |
| | | 1.42/1.42 (2 s, 6H), 1.05 (d, J = 6.7 Hz, 3H). |
| 342 | 541.4 | 1H NMR (400 MHz, DMSO) δ 8.31-8.28 (m, 2H), 8.09-7.96 (m, 2H), |
| | | 7.85 (d, J = 5.3 Hz, 1H), 6.47-6.45 (m 2H), 6.23 (d, J = 8.8 Hz, 1H), 4.43 |
| | | (s, 2H), 4.06-3.89 (m, 1H), 3.77-3.65 (m, 2H), 3.62-3.57 (m, 1H), 1.90- |
| | | 1.70 (m, 3H), 1.68-1.53 (m, 1H), 1.42/1.41 (2 s, 6H), 1.05 (d, J = 6.5 |
| | | Hz, 3H). |
| 343 | 563.2 | 1H NMR (400 MHz, DMSO) δ 8.32 (d, J = 8.8 Hz, 2H), 8.05 (d, J = 8.8 |
| | | Hz, 2H), 7.91 (d, J = 5.3 Hz, 1H), 6.70 (d, J = 9.0 Hz, 1H), 6.63 (s, 1H), |
| | | 6.58 (d, J = 5.3 Hz, 1H), 4.49 (s, 2H), 3.98-3.80 (m, 2H), 3.74-3.50 (m, |
| | | 2H), 1.97-1.83 (m, 1H), 1.78-1.61 (m, 1H), 1.45 (d, J = 2.8 Hz, 6H). |
| 344 | 537.3 | 1H NMR (400 MHz, DMSO) δ 8.34-8.28 (m, 2H), 8.06-8.01 (m, 2H), |
| | | 7.93 (d, J = 5.3 Hz, 1H), 7.56 (s, 1H), 7.33 (s, 1H), 6.61 (t, J = 5.6 Hz, |
| | | 1H), 6.53 (dd, J = 5.3, 1.4 Hz, 1H), 6.48 (s, 1H), 4.47 (s, 2H), 4.24 (d, J = |
| | | 5.5 Hz, 2H), 3.75 (s, 3H), 1.41 (s, 6H). |
| 345 | 436.4 | 1H NMR (400 MHz, DMSO) δ 7.87 (d, J = 5.3 Hz, 1H), 7.56-7.46 (m, |
| | | 2H), 7.37-7.27 (m, 2H), 6.89 (d, J = 7.1 Hz, 1H), 6.47 (d, J = 5.3 Hz, |
| | | 1H), 6.39 (s, 1H), 4.42 (s, 2H), 4.32-4.27 (m, 1H), 3.55 (t, J = 7.1 Hz, |
| | | 2H), 2.74 (t, J = 7.1 Hz, 2H), 2.20 (s, 3H), 1.36 (s, 6H), 1.29 (s, 9H). |
| 346 | 428.4 | ¹H NMR (400 MHz, DMSO) δ (ppm): 8.61 (d, J = 5.0 Hz, 1H), 8.10- |
| | | 8.03 (m, 2H), 7.95 (s, 1H), 7.51-7.48 (m, 4H), 7.47-7.42 (m, 1H), 7.40- |
| | | 7.33 (m, 3H), 4.68 (s, 2H), 1.41 (s, 6H), 1.30 (s, 9H). |
| 347 | 418.4 | ¹H NMR (400 MHz, DMSO) δ (ppm): 8.51 (d, J = 5.1 Hz, 1H), 7.92 (s, |
| | | 1H), 7.73 (s, 1H), 7.56-7.44 (m, 2H), 7.40-7.29 (m, 3H), 6.82 (d, J = |
| | | 2.1 Hz, 1H), 4.66 (s, 2H), 1.40 (s, 6H), 1.30 (s, 9H). |
| 348 | 496.3 | 1H NMR (400 MHz, DMSO) δ (ppm): 9.89 (s, 1H), 8.24 (d, J = 5.1 Hz, |
| | | 1H), 8.12 (s, 1H), 7.85 (d, J = 8.5 Hz, 2H), 7.68-7.61 (m, 2H), 7.16 (dd, |
| | | J = 5.1, 1.2 Hz, 1H), 4.64 (s, 2H), 3.10 (s, 2H), 2.27 (s, 6H), 1.41 (s, 6H). |
| 349 | 572.4 | 1H NMR (400 MHz, DMSO) δ (ppm): 8.75 (s, 1H), 8.02 (d, J = 5.1 Hz, |
| | | 1H), 7.86 (d, J = 8.5 Hz, 2H), 7.73-7.58 (m, 2H), 7.50 (d, J = 9.0 Hz, |
| | | 2H), 6.74-6.69 (m, 4H), 4.63 (t, J = 5.7 Hz, 1H), 4.50 (s, 2H), 3.79-3.62 |
| | | (m, 2H), 2.92-2.88 (m, 2H), 2.26 (s, 3H), 1.41 (s, 6H) |
| 350 | 534.5 | 1H NMR (400 MHz, DMSO) δ (ppm): 10.37 (s, 1H), 9.44 (s, 1H), 8.18 |
| | | (d, J = 5.3 Hz, 1H), 7.86 (d, J = 8.4 Hz, 2H), 7.81 (s, 1H), 7.66 (d, J = 8.5 |
| | | Hz, 2H), 7.42 (s, 1H), 7.35 (s, 1H), 7.01 (d, J = 5.1 Hz, 1H), 4.60 (s, 2H), |
| | | 3.77 (s, 3H), 1.41 (s, 6H). |
| 351 | 673.6 | 1H NMR (400 MHz, DMSO) δ (ppm): 10.41 (s, 1H), 9.33 (s, 1H), 8.20 |
| | | (d, J = 5.3 Hz, 1H), 7.86 (d, J = 8.5 Hz, 2H), 7.73-7.60 (m, 2H), 7.45 (s, |
| | | 1H), 7.19 (d, J = 2.4 Hz, 1H), 7.02 (dd, J = 5.4, 1.3 Hz, 1H), 6.91 (dd, J = |
| | | 8.6, 2.4 Hz, 1H), 6.75 (d, J = 8.7 Hz, 1H), 4.61 (s, 2H), 4.30 (p, J = 7.2 |
| | | Hz, 1H), 3.73 (s, 3H), 2.59-2.50 (m, 2H), 2.36-2.26 (m, 1H), 2.04 (s, |
| | | 6H), 1.85-1.72 (m, 2H), 1.41 (s, 6H). |
| 352 | 477.5 | 1H NMR (400 MHz, DMSO) δ (ppm): 11.51 (s, 1H), 9.97 (s, 1H), 8.10 |
| | | (d, J = 5.3 Hz, 1H), 7.87 (d, J = 8.5 Hz, 2H), 7.70-7.63 (m, 2H), 6.99 (s, |
| | | 1H), 6.81 (d, J = 5.4 Hz, 1H), 6.76 (s, 1H), 6.60 (s, 1H), 4.56 (s, 2H), 1.42 |
| | | (s, 6H). |
| 353 | 457.3 | 1H NMR (400 MHz, DMSO) δ 8.05-8.00 (m, 2H), 7.87 (d, J = 5.2 Hz, |
| | | 1H), 7.78-7.73 (m, 2H), 6.44 (d, J = 5.3 Hz, 1H), 6.40 (s, 1H), 6.25 (d, J = |
| | | 7.6 Hz, 1H), 4.43 (s, 2H), 4.00-3.89 (m, 1H), 2.90 (tt, J = 7.9, 4.8 Hz, |
| | | 1H), 1.40 (s, 6H), 1.17-1.02 (m, 10H). |
| 354 | 473.4 | 1H NMR (400 MHz, DMSO) δ 7.98-7.93 (m, 2H), 7.88 (br s, 1H), 7.83- |
| | | 7.77 (m, 2H), 6.45 (d, J = 4.6 Hz, 1H), 6.41 (s, 1H), 6.24 (d, J = 7.3 Hz, |
| | | 1H), 4.42 (s, 2H), 4.02-3.82 (m, 1H), 1.40 (s, 6H), 1.24 (s, 9H), 1.10 (d, |
| | | J = 6.4 Hz, 6H). |
| 355 | 497.1 | ¹H NMR (400 MHz, DMSO) δ (ppm): 8.30 (d, J = 8.8 Hz, 2H), 8.11- |
| | | 7.97 (m, 2H), 7.91 (d, J = 5.2 Hz, 1H), 6.74 (s, 1H), 6.57-6.54 (m, 2H), |
| | | 4.49 (s, 2H), 1.42 (s, 6H), 1.28 (s, 3H), 0.70-0.48 (m, 4H). |
| 356 | 483.2 | 1H NMR (400 MHz, DMSO) δ 8.29 (d, J = 8.8 Hz, 2H), 8.04-7.99 (m, |
| | | 2H), 7.91 (d, J = 5.2 Hz, 1H), 6.71 (d, J = 2.3 Hz, 1H), 6.59-6.57 (m, 2H), |
| | | 4.50 (s, 2H), 1.42 (s, 6H), 0.71-0.53 (m, 2H), 0.48-0.27 (m, 2H). |
| | | *missing C—H under DMSO signal |
| 357 | 501.3 | 1H NMR(400 MHz, DMSO) δ (ppm): 8.31 (d, J = 8.8 Hz, 2H), 8.04 (d, J = |
| | | 8.9 Hz, 2H), 7.95 (d, J = 5.1 Hz, 1H), 6.77 (d, J = 3.2 Hz, 1H), 6.62 (d, J = |
| | | 5.3 Hz, 1H), 6.61 (s, 1H), 4.88-4.64 (m, 1H), 4.51 (s, 2H), 2.66-2.57 |
| | | (m, 1H), 1.44 (s, 6H), 1.18-0.97 (m, 1H), 0.97-0.78 (m, 1H). |
| 358 | 502.1 | 1H NMR (400 MHz, DMSO) δ (ppm): 8.70 (s, 1H), 8.32 (s, 1H), 8.12 (d, |
| | | J = 4.8 Hz, 1H), 8.03-7.95 (m, 1H), 7.86 (d, J = 8.5 Hz, 2H), 7.69-7.61 |
| | | (m, 2H), 7.20 (d, J = 4.9 Hz, 1H), 6.75 (d, J = 4.7 Hz, 2H), 4.52 (s, 2H), |
| | | 2.19 (s, 3H), 1.42 (s, 6H). |
| 359 | 559.5 | 1H NMR (400 MHz, DMSO) δ (ppm): 9.39 (s, 1H), 8.73 (d, J = 2.5 Hz, |
| | | 1H), 8.38-8.32 (m, 1H), 8.15 (d, J = 5.1 Hz, 1H), 8.07 (d, J = 1.8 Hz, |
| | | 1H), 7.86 (d, J = 8.5 Hz, 2H), 7.75-7.61 (m, 2H), 6.94-6.82 (m, 2H), |
| | | 4.57 (s, 2H), 2.98 (s, 3H), 2.93 (s, 3H), 1.43 (s, 6H). |
| 360 | 516.5 | 1H NMR (400 MHz, DMSO) δ (ppm): 11.10 (s, 1H), 9.11 (dd, J = 2.3, 0.7 |
| | | Hz, 1H), 8.74 (dd, J = 4.8, 1.6 Hz, 1H), 8.38-8.27 (m, 2H), 8.23 (s, 1H), |
| | | 7.86 (d, J = 8.5 Hz, 2H), 7.72-7.59 (m, 2H), 7.53 (ddd, J = 8.0, 4.8, 0.8 |
| | | Hz, 1H), 7.24 (dd, J = 5.1, 1.4 Hz, 1H), 4.68 (s, 2H), 1.44 (s, 6H). |
| 361 | 468.2 | 1H NMR (400 MHz, DMSO) δ (ppm): 9.19 (s, 1H), 8.19 (s, 1H), 8.10 (d, |
| | | J = 5.3 Hz, 1H), 7.86 (d, J = 8.5 Hz, 2H), 7.71-7.50 (m, 2H), 7.25 (s, |
| | | 1H), 6.93 (dd, J = 5.3, 1.3 Hz, 1H), 4.56 (s, 2H), 2.71 (d, J = 4.6 Hz, 3H), |
| | | 1.40 (s, 6H). |
| 362 | 504.1 | 1H NMR (400 MHz, DMSO) δ (ppm): 11.24, 10.25 (2 singlets, 1H), 8.20 |
| | | (d, J = 5.4 Hz, H), 8.09 (d, J = 7.8 Hz, H), 7.86 (d, J = 8.5 Hz, 2H), 7.69- |
| | | 7.64 (m, 2H), 7.46 (d, J = 8.1 Hz, 2H), 7.40-7.31 (m, 2H), 7.13 (t, J = |
| | | 7.6, 1.0 Hz, 2H), 4.60 (s, 212H), 1.43 (s, 6H). |
| 363 | 560.4 | 1H NMR (400 MHz, DMSO) δ (ppm): 10.46 (s, 1H), 9.36 (s, 1H), 8.20 |
| | | (d, J = 5.3 Hz, 1H), 7.86 (d, J = 8.5 Hz, 2H), 7.70-7.63 (m, 2H), 7.47- |
| | | 7.37 (m, 3H), 7.02 (dd, J = 5.3, 1.3 Hz, 1H), 6.92-6.82 (m, 2H), 4.61 (s, |
| | | 2H), 3.71 (s, 3H), 1.42 (s, 6H). |
| 364 | 560.5 | 1H NMR (400 MHz, DMSO) δ (ppm): 10.53 (s, 1H), 9.39 (s, 1H), 8.22 |
| | | (d, J = 5.3 Hz, 1H), 7.86 (d, J = 8.5 Hz, 2H), 7.74-7.62 (m, 2H), 7.50 (s, |
| | | 1H), 7.24 (t, J = 2.2 Hz, 1H), 7.19 (t, J = 8.1 Hz, 1H), 7.04 (dd, J = 5.3, |
| | | 1.4 Hz, 1H), 6.98 (dd, J = 8.1, 1.2 Hz, 1H), 6.64-6.55 (m, 1H), 4.62 (s, |
| | | 2H), 3.73 (s, 3H), 1.42 (s, 6H). |
| 365 | 454.1 | 1H NMR (400 MHz, DMSO-d6) δ (ppm): 10.95 (s, 1H), 8.82 (d, J = 1.2 |
| | | Hz, 1H), 8.12 (d, J = 0.8 Hz, 1H), 7.89-7.84 (m, 2H), 7.65-7.61 (m, |
| | | 2H), 4.67 (s, 2H), 2.13 (s, 3H), 1.45 (s, 6H). |
| 366 | 544.5 | 1H NMR (400 MHz, DMSO) δ (ppm): 10.98 (s, 1H), 9.73 (s, 1H), 8.23 |
| | | (d, J = 5.3 Hz, 1H), 8.03 (d, J = 7.5 Hz, 1H), 7.88 (d, J = 8.5 Hz, 2H), 7.74- |
| | | 7.63 (m, 2H), 7.27 (s, 1H), 7.21 (d, J = 7.4 Hz, 1H), 7.16 (t, J = 7.7 Hz, |
| | | 1H), 7.05 (dd, J = 5.4, 1.3 Hz, 1H), 6.96 (td, J = 7.4, 1.1 Hz, 1H), 4.62 (s, |
| | | 2H), 2.31 (s, 3H), 1.43 (s, 6H). |
| 367 | 582.5 | 1H NMR (400 MHz, DMSO) δ 11.35 (s, 1H), 8.42 (d, J = 5.3 Hz, 1H), |
| | | 7.87 (d, J = 8.5 Hz, 2H), 7.71-7.64 (m, 2H), 7.55 (d, J = 7.7 Hz, 2H), |
| | | 7.45 (d, J = 4.6 Hz, 1H), 7.36 (t, J = 8.0 Hz, 2H), 7.14 (d, J = 5.3 Hz, 1H), |
| | | 7.07 (t, J = 7.4 Hz, 1H), 4.65 (s, 2H), 1.44 (s, 6H). |
| 368 | 548.4 | 1H NMR (400 MHz, DMSO) δ (ppm): 10.65 (s, 1H), 9.45 (s, 1H), 8.22 |
| | | (d, J = 5.3 Hz, 1H), 7.86 (d, J = 8.5 Hz, 2H), 7.69-7.63 (m, 2H), 7.56- |
| | | 7.49 (m, 2H), 7.44 (s, 1H), 7.18-7.09 (m, 2H), 7.04 (dd, J = 5.3, 1.2 Hz, |
| | | 1H), 4.61 (s, 2H), 1.42 (s, 6H). |
| 369 | 479.2 | ¹H NMR (400 MHz, DMSO) δ (ppm): 7.88-8.84 (m, 3H), 7.75-7.56 (m, |
| | | 2H), 6.45-6.40 (m, 3H), 4.42 (s, 2H), 4.08-4.03 (m, 1H), 1.89-1.85 (m, |
| | | 2H), 1.65-1.62 (m, 2H), 1.52-1.49 (m, 2H), 1.43-1.34 (m, 8H). |
| 370 | 496.3 | ¹H NMR (400 MHz, DMSO) δ (ppm): 9.04 (s, 1H), 8.10-8.08 (m, 2H), |
| | | 7.86 (d, J = 8.5 Hz, 2H), 7.71-7.59 (m, 2H), 7.32 (s, 1H), 6.93 (dd, J = |
| | | 5.3, 1.4 Hz, 1H), 4.56 (s, 2H), 3.84-3.76 (m, 1H), 1.40 (s, 6H), 1.11 (d, J = |
| | | 6.5 Hz, 6H). |
| 371 | 544.4 | 1H NMR (400 MHz, DMSO) δ (ppm): 10.52 (s, 1H), 9.38 (s, 1H), 8.21 |
| | | (d, J = 5.3 Hz, 1H), 7.86 (d, J = 8.5 Hz, 2H), 7.72-7.63 (m, 2H), 7.44 (s, |
| | | 1H), 7.39 (d, J = 8.4 Hz, 2H), 7.09 (d, J = 8.4 Hz, 2H), 7.03 (dd, J = 5.4, |
| | | 1.2 Hz, 1H), 4.61 (s, 2H), 2.24 (s, 3H), 1.42 (s, 6H). |
| 372 | 544.5 | 1H NMR (400 MHz, DMSO) δ (ppm): 10.52 (s, 1H), 9.38 (s, 1H), 8.19 |
| | | (d, J = 5.3 Hz, 1H), 7.83 (d, J = 8.5 Hz, 2H), 7.68-7.59 (m, 2H), 7.42 (s, |
| | | 1H), 7.32-7.23 (m, 2H), 7.13 (t, J = 7.8 Hz, 1H), 7.00 (dd, J = 5.3, 1.2 |
| | | Hz, 1H), 6.79 (d, J = 7.5 Hz, 1H), 4.58 (s, 2H), 2.24 (s, 3H), 1.38 (s, 6H). |
| 373 | 548.2 | 1H NMR (400 MHz, DMSO) δ (ppm): 10.79 (s, 1H), 9.50 (s, 1H), 8.23 |
| | | (d, J = 5.3 Hz, 1H), 7.86 (d, J = 8.5 Hz, 2H), 7.72-7.63 (m, 2H), 7.56 (dt, |
| | | J = 11.9, 2.3 Hz, 1H), 7.48 (s, 1H), 7.38-7.25 (m, 1H), 7.19 (dd, J = 8.2, |
| | | 1.1 Hz, 1H), 7.06 (dd, J = 5.3, 1.3 Hz, 1H), 6.83 (td, J = 8.2, 1.9 Hz, 1H), |
| | | 4.62 (s, 2H), 1.42 (s, 6H). |
| 374 | 558.3 | 1H NMR (400 MHz, DMSO) δ (ppm): 9.16 (s, 1H), 8.45 (d, J = 6.4 Hz, |
| | | 1H), 8.11 (d, J = 5.3 Hz, 1H), 7.86 (d, J = 8.5 Hz, 2H), 7.72-7.59 (m, |
| | | 2H), 7.33 (s, 1H), 6.95 (dd, J = 5.3, 1.4 Hz, 1H), 4.57 (s, 2H), 4.26-4.13 |
| | | (m, 1H), 2.47-2.39 (m, 1H), 2.26-1.93 (m, 4H), 1.75-1.61 (m, 1H), |
| | | 1.40 (s, 6H) |
| 375 | 548.2 | 1H NMR (400 MHz, DMSO) δ (ppm): 11.06 (s, 1H), 9.79 (s, 1H), 8.26- |
| | | 8.15 (m, 2H), 7.86 (d, J = 8.5 Hz, 2H), 7.71-7.62 (m, 2H), 7.37 (s, 1H), |
| | | 7.30-7.20 (m, 1H), 7.14 (t, J = 7.7 Hz, 1H), 7.10-6.99 (m, 2H), 4.62 (s, |
| | | 2H), 1.42 (s, 6H). |
| 376 | 555.5 | 1H NMR (400 MHz, DMSO) δ (ppm): 10.97 (s, 1H), 9.61 (s, 1H), 8.24 |
| | | (d, J = 5.3 Hz, 1H), 7.86 (d, J = 8.5 Hz, 2H), 7.80-7.60 (m, 6H), 7.51 (s, |
| | | 1H), 7.08 (dd, J = 5.3, 1.3 Hz, 1H), 4.62 (s, 2H), 1.42 (s, 6H) |
| 377 | 564.5 | 1H NMR (400 MHz, DMSO) δ (ppm): 10.72 (s, 1H), 9.48 (s, 1H), 8.22 |
| | | (d, J = 5.3 Hz, 1H), 7.86 (d, J = 8.5 Hz, 2H), 7.70-7.63 (m, 2H), 7.60- |
| | | 7.51 (m, 2H), 7.46 (s, 1H), 7.41-7.29 (m, 2H), 7.05 (dd, J = 5.3, 1.3 Hz, |
| | | 1H), 4.61 (s, 2H), 1.42 (s, 6H). |
| 378 | 465.3 | ¹H NMR (400 MHz, DMSO) δ (ppm): 7.87-7.84 (m, 3H), 7.64 (d, J = 8.5 |
| | | Hz, 2H), 6.71-6.69 (m, 1H), 6.48 (s, 1H), 6.37 (s, 1H), 4.42 (s, 2H), 4.26- |
| | | 4.20 (m, 1H), 2.31-2.11 (m, 2H), 1.84-1.79 (m, 2H), 1.71-1.53 (m, |
| | | 2H), 1.38 (s, 6H). |
| 379 | 454.3 | 1H NMR (400 MHz, DMSO-d6) δ (ppm): 8.35 (s, 1H), 7.90-7.84 (m, |
| | | 2H), 7.68-7.63 (m, 2H), 7.21 (d, J = 7.5 Hz, 1H), 6.43 (s, 1H), 4.40 (s, |
| | | 2H), 4.07 (br s, 1H), 1.44 (s, 6H), 1.13 (d, J = 6.5 Hz, 6H). |
| 380 | 429.3 | 1H NMR (400 MHz, DMSO-d6) δ (ppm): 7.89-7.83 (m, 2H), 7.69 (d, J = |
| | | 5.2 Hz, 1H), 7.67-7.63 (m, 2H), 6.61 (t, J = 5.0 Hz, 1H), 6.19 (s, 2H), |
| | | 4.58 (s, 2H), 1.42 (s, 6H). |
| 381 | 467.6 | 1H NMR (400 MHz, DMSO) δ (ppm): 7.89-7.80 (m, 3H), 7.69-7.61 |
| | | (m, 2H), 6.46-6.36 (m, 2H), 6.20 (d, J = 8.0 Hz, 1H), 4.41 (s, 2H), 3.89- |
| | | 3.72 (m, 1H), 1.56-1.41 (m, 2H), 1.39 (s, 6H), 1.06 (d, J = 6.5 Hz, 3H), |
| | | 0.84 (t, J = 7.4 Hz, 3H). |
| 382 | 471.3 | 1H NMR (400 MHz, DMSO-d6) δ 7.89-7.83 (m, 2H), 7.75 (d, J = 5.2 |
| | | Hz, 1H), 7.68-7.62 (m, 2H), 6.57 (t, J = 5.0 Hz, 1H), 6.33 (d, J = 7.7 Hz, |
| | | 1H), 4.57 (s, 2H), 4.23-4.11 (m, 1H), 1.43 (s, 6H), 1.16 (d, J = 6.5 Hz, 6H). |
| 383 | 471.3 | 1H NMR (400 MHz, DMSO-d6) δ 7.89-7.84 (m, 2H), 7.69-7.64 (m, |
| | | 2H), 6.75 (d, J = 7.5 Hz, 1H), 6.31 (s, 1H), 6.11 (s, 1H), 4.46 (s, 2H), 3.91- |
| | | 3.81 (m, 1H), 1.42 (s, 6H), 1.11 (d, J = 6.4 Hz, 6H). |
| 384 | 471.3 | 1H NMR (400 MHz, DMSO-d6) δ (ppm): 7.90-7.85 (m, 3H), 7.68- |
| | | 7.64 (m, 2H), 6.46 (d, J = 5.3 Hz, 1H), 6.21 (d, J = 7.4 Hz, 1H), 4.53 (s, |
| | | 2H), 3.94-3.84 (m, 1H), 1.45 (s, 6H), 1.10 (d, J = 6.4 Hz, 6H). |
| 385 | 477.2 | 1H NMR (400 MHz, DMSO) δ 7.99 (d, J = 2.1 Hz, 1H), 7.86-7.80 (m, |
| | | 2H), 7.65-7.61 (m, 2H), 7.51 (d, J = 2.3 Hz, 1H), 6.02 (s, 2H), 4.38 (s, |
| | | 2H), 2.81 (hept, J = 6.9 Hz, 1H), 1.36 (s, 6H), 1.19 (d, J = 6.9 Hz, 6H). |
| 386 | 454.2 | 1H NMR (400 MHz, DMSO) δ 8.42 (d, J = 1.9 Hz, 1H), 7.91-7.81 (m, |
| | | 2H), 7.69-7.61 (m, 2H), 6.69 (d, J = 1.8 Hz, 1H), 6.55 (d, J = 7.4 Hz, |
| | | 1H), 4.48 (s, 2H), 4.15-4.01 (m, 1H), 1.41 (s, 6H), 1.15 (d, J = 6.4 Hz, 6H). |
| 387 | 467.3 | 1H NMR (400 MHz, DMSO) δ 7.91-7.77 (m, 3H), 7.69-7.57 (m, 2H), |
| | | 6.44 (s, 1H), 6.41 (d, J = 5.3 Hz, 1H), 6.10 (s, 1H), 4.39 (s, 2H), 1.40 (s, |
| | | 6H), 1.34 (s, 9H). |
| 388 | 489 | 1H NMR (400 MHz, DMSO-D6) δ (ppm): 7.90-7.84 (m, 3H), 7.70- |
| | | 7.65 (m, 2H), 6.68 (d, J = 5.1 Hz, 1H), 6.27 (s, 2H), 4.50 (s, 2H), 1.44 (s, 6H). |
| 389 | 487.3 | 1H NMR (400 MHz, DMSO) δ 7.99 (dd, J = 5.2, 2.1 Hz, 1H), 7.88-7.83 |
| | | (m, 2H), 7.68-7.63 (m, 2H), 7.14 (s, 2H), 6.67 (dd, J = 5.2, 3.0 Hz, 1H), |
| | | 4.58 (s, 2H), 1.90 (s, 3H), 1.87 (s, 3H), 1.47 (s, 6H). |
| 390 | 531.2 | 1H NMR (400 MHz, DMSO-d6) δ (ppm): 7.96 (d, J = 5.1 Hz, 1H), 7.89- |
| | | 7.84 (m, 2H), 7.70-7.64 (m, 2H), 6.68 (d, J = 5.1 Hz, 1H), 5.74 (d, J = |
| | | 7.9 Hz, 1H), 4.50 (s, 2H), 4.26-4.15 (m, 1H), 1.44 (s, 6H), 1.19 (d, J = |
| | | 6.5 Hz, 6H). |
| 391 | 436.3 | 1H NMR (400 MHz, DMSO-D6) δ (ppm): 8.14 (d, J = 5.2 Hz, 1H), 7.90- |
| | | 7.84 (m, 2H), 7.69-7.62 (m, 2H), 6.95 (s, 2H), 6.77 (d, J = 5.2 Hz, 1H), |
| | | 4.62 (s, 2H), 1.46 (s, 6H). |
| 392 | 454.3 | 1H NMR (400 MHz, DMSO-D6) δ 8.81 (dd, J = 2.5, 0.5 Hz, 1H), 8.10 |
| | | (dd, J = 8.4, 2.6 Hz, 1H), 7.95 (d, J = 8.5 Hz, 1H), 7.89 (d, J = 5.3 Hz, |
| | | 1H), 6.47 (dd, J = 5.3, 1.3 Hz, 1H), 6.41 (s, 1H), 6.25 (d, J = 7.6 Hz, 1H), |
| | | 4.44 (s, 2H), 4.03-3.90 (m, 1H), 1.42 (s, 6H), 1.11 (d, J = 6.4 Hz, 6H). |
| 393 | 427.2 | 1H NMR (400 MHz, DMSO) δ (ppm): 8.02 (d, J = 8.6 Hz, 2H), 7.87- |
| | | 7.80 (m, 3H), 6.49 (dd, J = 5.3, 1.2 Hz, 1H), 6.42 (s, 1H), 5.87 (s, 2H), |
| | | 4.44 (s, 2H), 1.40 (s, 6H). |
| 394 | 473 | 1H NMR (500 MHz, DMSO) δ (ppm): 8.30 (d, J = 8.8 Hz, 2H), 8.04 (d, J = |
| | | 9.0 Hz, 2H), 7.64 (d, J = 5.1 Hz, 1H), 6.58 (d, J = 5.1 Hz, 1H), 5.88 (s, |
| | | 2H), 4.55 (s, 2H), 3.72 (s, 3H), 1.43 (s, 6H). |
| 395 | 357.2 | 1H NMR (400 MHz, DMSO) δ 7.82 (d, J = 5.2 Hz, 1H), 7.35 (app. s, 4H), |
| | | 6.47 (dd, J = 5.3, 1.2 Hz, 1H), 6.40 (s, 1H), 5.86 (s, 2H), 4.40 (s, 2H), 2.49 |
| | | (s, 3H), 1.36 (s, 6H). |
| 396 | 412.1 | 1H NMR (400 MHz, DMSO-d6) δ (ppm): 8.30 (d, J = 0.9 Hz, 1H), 7.89- |
| | | 7.84 (m, 2H), 7.68-7.63 (m, 2H), 6.81 (s, 2H), 6.44 (s, 1H), 4.41 (s, 2H), |
| | | 1.44 (s, 6H). |
| 397 | 531.4 | 1H NMR (400 MHz, DMSO) δ (ppm): 10.77 (s, 1H), 9.57 (s, 1H), 8.67 |
| | | (d, J = 2.5 Hz, 1H), 8.27-8.19 (m, 2H), 8.01 (ddd, J = 8.4, 2.6, 1.5 Hz, |
| | | 1H), 7.87 (d, J = 8.5 Hz, 2H), 7.70-7.63 (m, 2H), 7.48 (s, 1H), 7.33 (dd, |
| | | J = 8.3, 4.7 Hz, 1H), 7.07 (dd, J = 5.3, 1.3 Hz, 1H), 4.63 (s, 2H), 1.43 (s, 6H). |
| 398 | 531.4 | 1H NMR (400 MHz, DMSO) δ (ppm): 10.88 (s, 1H), 9.64 (s, 1H), 8.41 |
| | | (d, J = 6.3 Hz, 2H), 8.25 (d, J = 5.3 Hz, 1H), 7.87 (d, J = 8.5 Hz, 2H), 7.72- |
| | | 7.62 (m, 2H), 7.60-7.50 (m, 3H), 7.10 (dd, J = 5.3, 1.2 Hz, 1H), 4.64 |
| | | (s, 2H), 1.43 (s, 6H). |
| 399 | 489.2 | ¹H NMR (400 MHz, DMSO) δ (ppm): 9.82 (s, 1H), 8.50 (d, J = 4.8 Hz, |
| | | 2H), 8.25 (s, 1H), 8.20 (d, J = 5.1 Hz, 1H), 7.85 (d, J = 8.5 Hz, 2H), 7.71- |
| | | 7.58 (m, 2H), 7.01 (dd, J = 5.1, 1.3 Hz, 1H), 6.94-6.91 (m, 1H), 4.63 (s, |
| | | 2H), 1.44 (s, 6H). |
| 400 | 399.2 | 1H NMR (400 MHz, DMSO-d6) δ 12-59 (br s, 1H), 7.86-7.82 (m, 2H), |
| | | 7.66-7.58 (m, 2H), 7.52 (br s, 2H), 3.50-3.42 (m, 2H), 2.83-2.76 (m, |
| | | 2H), 1.41 (s, 6H). |
| 401 | 380.3 | 1H NMR (400 MHz, DMSO-d6) δ 7.83 (d, J = 5.3 Hz, 1H), 7.05 (d, J = |
| | | 7.7 Hz, 1H), 6.52 (dd, J = 7.—, 1.8 Hz, 1H), 6.47-6.44 (m, 2H), 6.40 (s, |
| | | 1H), 5.89 (s, 2H), 5.70 (s, 1H), 4.41 (s, 2H), 3.23 (d, J = 1.6 Hz, 2H), 1.35 |
| | | (s, 6H), 1.24 (s, 6H). Contains a trace of acetonitrile at 2.07 ppm. |
| 402 | 406.4 | 1H NMR (500 MHz, DMSO) δ 7.87 (d, J = 5.4 Hz, 1H), 7.14 (dd, J = 4.7, |
| | | 3.0 Hz, 2H), 7.04 (dd, J = 7.9, 1.6 Hz, 1H), 6.55 (d, J = 5.4 Hz, 1H), 6.51 |
| | | (s, 1H), 6.14 (s, 2H), 4.47 (s, 2H), 3.23 (s, 3H), 1.68 (dd, J = 7.7, 3.9 Hz, |
| | | 2H), 1.57 (dd, J = 7.6, 3.7 Hz, 2H), 1.42 (s, 6H). |
| 403 | 519.1 | 1H NMR (400 MHz, DMSO) δ 7.96 (d, J = 1.8 Hz, 1H), 7.84 (d, J = 8.5 |
| | | Hz, 2H), 7.67-7.59 (m, 2H), 7.49 (s, 1H), 6.77 (s, 1H), 4.52-4.37 (m, |
| | | 2H), 3.69 (dd, J = 10.6, 1.1 Hz, 1H), 3.39 (d, J = 10.8 Hz, 1H), 1.47 (s, |
| | | 3H), 1.36 (s, 3H), 1.35 (s, 3H). |
| 405 | 465.2 | 1H NMR (400 MHz, DMSO) δ 7.86-7.82 (m, 2H), 7.78 (d, J = 1.7 Hz, |
| | | 1H), 7.68-7.61 (m, 2H), 7.27 (d, J = 1.9 Hz, 1H), 6.37 (s, 1H), 4.41 (s, |
| | | 2H), 3.18 (s, 2H), 1.35 (s, 6H), 1.20 (s, 6H). |
| 406 | 449.2 | 1H NMR (400 MHz, DMSO) δ 11.24 (s, 1H), 8.27 (d, J = 2.0 Hz, 1H), |
| | | 7.95 (d, J = 1.8 Hz, 1H), 7.87-7.79 (m, 2H), 7.69-7.62 (m, 2H), 7.20 |
| | | (d, J = 1.0 Hz, 1H), 4.68 (s, 2H), 2.22 (d, J = 1.0 Hz, 3H), 1.37 (s, 6H). |
| 407 | 407.3 | 1H NMR (400 MHz, DMSO) δ 11.69 (s, 1H), 8.11 (d, J = 4.8 Hz, 1H), |
| | | 7.51-7.45 (m, 2H), 7.43 (dd, J = 3.4, 2.6 Hz, 1H), 7.25-7.14 (m, 2H), |
| | | 7.09 (d, J = 4.9 Hz, 1H), 6.55 (dd, J = 3.5, 1.8 Hz, 1H), 4.60 (s, 2H), 1.36 |
| | | (s, 6H), 1.25 (s, 9H). |
| 408 | 460.2 | 1H NMR (400 MHz, DMSO) δ 8.29 (d, J = 8.8 Hz, 2H), 8.06-7.99 (m, |
| | | 2H), 7.89 (d, J = 5.5 Hz, 1H), 6.57 (d, J = 5.5 Hz, 1H), 6.52 (s, 1H), 4.49 |
| | | (s, 2H), 1.41 (s, 6H). N—H missing |

### II. Biologic and Pharmacologic Assays and Methods

The activity of the compounds as IGF-1R inhibitors is illustrated in the following assays.

### Example B-1: IGF-1R Assay

50K PC3 cells were seeded on day 1 in 50 μL of media in 96-well plate and incubated overnight at 37° C., 5% CO₂. On day 2, all cells were expected to be at 80-90% confluency. Cells were starved for two hours by changing the media to 90 μL of EMEM serum-free media and incubated for 2 hours. Next, cells were treated with 10 μL of 10× concentrated and 3× serially diluted compounds for two hours. It makes 1× of compounds in 100 μL media. This was followed by stimulation of cells with 11×IGF1 for 30 min in 10 μL of serum-free EMEM media at the final concentration of 200 ng/mL.

The media was removed and replaced by 50 μL of 1× lysis buffer from the AlphaLISA kit (with 1× of cOmplete™ protease inhibitor). The plates were sealed, and cells were allowed to lyse for 10 min in the shaker before storing the plate at −20° C. until used. The AlphaLISA assay with processed cell lysates were performed as described in the protocol. Briefly, 10 μL of the undiluted sample were loaded to 384 well Optiplates and 5 μL of the acceptor mix (as described in the AlphaLISA kit) was added to each well. The plates were sealed and briefly vortexed followed by the incubation at RT for 3 hours. In the dark, 5 μL of donor mix was added to each well. All plates were sealed and stored at RT overnight. The plates were read in EnVision reader using standard AlphaLISA settings.

Compound activity was analyzed using CBIS data analysis suite (Cheminnovation, CA). Percentage inhibition was calculated using the following formula: % I-hibition=100%×(1−(mea−RLU of test sample−mean RLU of vehicle control)/(mean-RLU of EC80 control−mean RLU of vehicle control)). The activity for representative examples of the invention is shown in the following table, wherein A: IC₅₀≤500 nM; B: 500 nM<IC₅₀≤2 μM; C: 2 μM<IC₅₀≤10 μM; D: IC₅₀>10 μM.

TABLE B1
Biological Activity against IGF-1R

| Cmpd # | IC₅₀ |
|---|---|
| 1 | A |
| 2 | A |
| 3 | A |
| 4 | A |
| 5 | A |
| 6 | A |
| 7 | A |
| 8 | A |
| 9 | A |
| 10 | A |
| 11 | A |
| 12 | A |
| 13 | A |
| 14 | D |
| 15 | D |
| 16 | A |
| 17 | A |
| 18 | A |
| 19 | A |
| 20 | A |
| 21 | A |
| 22 | A |
| 23 | A |
| 24 | A |
| 25 | A |
| 80 | B |
| 81 | C |
| 82 | A |
| 83 | A |
| 84 | A |
| 85 | B |
| 86 | B |
| 87 | B |
| 88 | B |
| 89 | A |
| 90 | B |
| 91 | C |
| 92 | B |
| 93 | A |
| 94 | A |
| 95 | A |
| 96 | B |
| 97 | A |
| 98 | A |
| 99 | B |
| 100 | B |
| 101 | A |
| 102 | B |
| 103 | A |
| 104 | A |
| 105 | A |
| 106 | A |
| 107 | A |
| 108 | A |
| 109 | A |
| 110 | B |
| 111 | C |
| 112 | C |
| 113 | A |
| 114 | A |
| 115 | A |
| 116 | A |
| 117 | A |
| 118 | A |
| 119 | A |
| 120 | A |
| 121 | A |
| 122 | D |
| 123 | B |
| 124 | A |
| 125 | A |
| 126 | B |
| 127 | A |
| 128 | A |
| 129 | A |
| 130 | A |
| 131 | A |
| 132 | C |
| 133 | B |
| 134 | A |
| 135 | A |
| 136 | B |
| 137 | A |
| 138 | A |
| 139 | A |
| 178 | A |
| 179 | A |
| 185 | C |
| 186 | C |
| 187 | C |
| 188 | A |
| 189 | A |
| 190 | C |
| 191 | A |
| 254 | B |
| 255 | B |
| 256 | B |
| 257 | B |
| 258 | C |
| 263 | A |
| 264 | A |
| 265 | A |
| 266 | A |
| 267 | A |
| 268 | A |
| 269 | A |
| 270 | A |
| 271 | B |
| 272 | A |
| 273 | A |
| 274 | B |
| 275 | A |
| 276 | A |
| 277 | A |
| 278 | A |
| 289 | B |
| 290 | D |
| 291 | B |
| 292 | B |
| 293 | C |
| 294 | D |
| 295 | C |
| 296 | D |
| 297 | A |
| 298 | D |
| 299 | C |
| 300 | C |
| 301 | A |
| 302 | A |
| 303 | A |
| 304 | B |
| 305 | C |
| 306 | A |
| 307 | A |
| 308 | A |
| 309 | A |
| 310 | A |
| 311 | A |
| 312 | B |
| 313 | B |
| 314 | D |
| 315 | A |
| 316 | A |
| 317 | A |
| 318 | A |
| 319 | A |
| 345 | B |
| 346 | B |
| 347 | A |
| 348 | A |
| 349 | A |
| 350 | A |
| 351 | A |
| 352 | A |
| 358 | A |
| 359 | A |
| 360 | A |
| 361 | A |
| 362 | B |
| 363 | A |
| 364 | A |
| 365 | A |
| 366 | A |
| 367 | B |
| 368 | A |
| 369 | A |
| 370 | A |
| 371 | A |
| 372 | A |
| 373 | A |
| 374 | A |
| 375 | A |
| 376 | A |
| 377 | B |
| 378 | A |
| 379 | B |
| 380 | A |
| 381 | A |
| 382 | C |
| 383 | C |
| 384 | A |
| 385 | B |
| 386 | C |
| 387 | B |
| 388 | A |
| 389 | B |
| 390 | C |
| 391 | A |
| 392 | B |
| 393 | A |
| 394 | A |
| 395 | C |
| 396 | A |
| 397 | A |
| 398 | A |
| 399 | A |
| 400 | C |
| 401 | A |
| 402 | C |
| 405 | C |
| 406 | C |

Example B-2: IGF-1R Assay

PathHunter cell lines were expanded from freezer stocks according to standard procedures. Cells were seeded in a total volume of 20 μL into white walled, 384-well microplates and incubated for the appropriate time prior to testing.

Cells are pre-incubated with compound followed by EC80 agonist challenge as follows:
R. 1. Intermediate dilution of sample stocks was performed to generate 5× sample in assay buffer.
2. 5 μL of 5× sample was added to cells and incubated at 37° C. for 60 minutes. Vehicle concentration was 1%.
3. 5 IL of 6× EC80 agonist in assay buffer was added to the cells and incubated at room temperature for 3 hours.

Assay signal was generated through a single addition of 12.5 or 15 μL (50% v/v) of PathHunter Detection reagent cocktail for agonist and antagonist assays respectively, followed by a one hour incubation at room temperature. Microplates were read following signal generation with a PerkinElmer Envision™ instrument for chemilumine-scent signal detection.

Compound activity was analyzed using CBIS data analysis suite (ChemInnovation, CA). Percentage inhibition was calculated using the following formula: % Inhibition=100%×(1−(mean RLU of test sample−mean RLU of vehicle control)/(mean RLU of EC80 control−mean RLU of vehicle control)). The activity for representative examples of the invention is shown in the following table, wherein A: IC₅₀≤500 nM; B: 500 nM<IC₅₀≤2 μM; C: 2 μM<IC₅₀≤10 μM; D: IC₅₀>10 μM.

TABLE B2
Biological Activity against IGF-1R

| Cmpd # | IC₅₀ |
|---|---|
| 1 | A |
| 12 | A |
| 13 | A |
| 16 | A |
| 17 | A |
| 18 | A |
| 19 | A |
| 20 | A |
| 21 | A |
| 22 | A |
| 23 | A |
| 24 | A |
| 25 | A |
| 26 | A |
| 27 | A |
| 28 | A |
| 29 | A |
| 30 | A |
| 31 | A |
| 32 | A |
| 33 | A |
| 34 | A |
| 35 | A |
| 36 | A |
| 37 | A |
| 38 | A |
| 39 | A |
| 40 | A |
| 41 | A |
| 42 | D |
| 43 | A |
| 44 | D |
| 45 | A |
| 46 | A |
| 47 | C |
| 48 | A |
| 49 | A |
| 50 | A |
| 51 | A |
| 52 | A |
| 53 | B |
| 54 | A |
| 55 | A |
| 56 | A |
| 57 | A |
| 58 | A |
| 59 | A |
| 60 | A |
| 61 | A |
| 62 | D |
| 63 | B |
| 64 | B |
| 65 | D |
| 66 | D |
| 67 | D |
| 68 | B |
| 69 | A |
| 70 | C |
| 71 | B |
| 72 | A |
| 74 | A |
| 75 | A |
| 76 | C |
| 77 | A |
| 78 | A |
| 79 | D |
| 84 | A |
| 88 | C |
| 93 | A |
| 97 | A |
| 98 | A |
| 104 | B |
| 106 | A |
| 107 | A |
| 109 | A |
| 110 | C |
| 111 | C |
| 114 | A |
| 118 | A |
| 119 | A |
| 120 | A |
| 121 | A |
| 122 | A |
| 123 | B |
| 124 | A |
| 125 | A |
| 126 | B |
| 127 | A |
| 128 | A |
| 129 | A |
| 130 | A |
| 131 | A |
| 132 | B |
| 133 | B |
| 134 | B |
| 135 | A |
| 136 | B |
| 137 | A |
| 138 | A |
| 139 | A |
| 140 | B |
| 141 | B |
| 142 | A |
| 143 | B |
| 144 | B |
| 145 | A |
| 146 | B |
| 147 | A |
| 148 | A |
| 149 | A |
| 150 | A |
| 151 | B |
| 152 | B |
| 153 | A |
| 154 | B |
| 155 | A |
| 156 | A |
| 157 | A |
| 158 | B |
| 159 | A |
| 160 | A |
| 161 | A |
| 162 | A |
| 163 | A |
| 164 | A |
| 165 | A |
| 166 | B |
| 167 | A |
| 168 | A |
| 169 | A |
| 170 | A |
| 171 | A |
| 172 | A |
| 173 | A |
| 174 | A |
| 175 | A |
| 176 | A |
| 177 | A |
| 180 | D |
| 181 | A |
| 182 | A |
| 183 | A |
| 184 | A |
| 185 | C |
| 186 | C |
| 187 | C |
| 188 | A |
| 189 | A |
| 190 | C |
| 191 | A |
| 192 | D |
| 193 | C |
| 194 | A |
| 195 | A |
| 196 | A |
| 197 | C |
| 198 | A |
| 199 | A |
| 200 | A |
| 201 | A |
| 202 | A |
| 203 | A |
| 204 | A |
| 205 | A |
| 206 | B |
| 207 | A |
| 208 | A |
| 209 | A |
| 210 | A |
| 211 | A |
| 212 | A |
| 213 | A |
| 214 | A |
| 215 | A |
| 216 | A |
| 217 | A |
| 218 | A |
| 219 | A |
| 220 | A |
| 221 | A |
| 222 | A |
| 223 | A |
| 224 | A |
| 225 | A |
| 226 | A |
| 227 | A |
| 228 | A |
| 229 | A |
| 230 | A |
| 231 | A |
| 232 | A |
| 233 | A |
| 234 | A |
| 235 | B |
| 236 | A |
| 237 | A |
| 238 | A |
| 239 | A |
| 240 | A |
| 241 | A |
| 242 | A |
| 243 | A |
| 244 | A |
| 245 | A |
| 246 | A |
| 247 | A |
| 248 | A |
| 249 | A |
| 250 | D |
| 251 | D |
| 252 | D |
| 253 | A |
| 255 | C |
| 256 | C |
| 258 | C |
| 259 | A |
| 260 | C |
| 261 | D |
| 262 | D |
| 264 | B |
| 267 | A |
| 277 | A |
| 278 | B |
| 279 | A |
| 280 | A |
| 281 | A |
| 282 | A |
| 283 | D |
| 284 | B |
| 285 | B |
| 286 | C |
| 287 | C |
| 288 | D |
| 298 | D |
| 299 | C |
| 310 | A |
| 316 | A |
| 317 | A |
| 318 | A |
| 319 | A |
| 320 | A |
| 321 | B |
| 322 | B |
| 323 | C |
| 324 | B |
| 325 | A |
| 326 | C |
| 327 | C |
| 328 | D |
| 329 | B |
| 330 | B |
| 331 | B |
| 332 | C |
| 333 | B |
| 334 | B |
| 335 | B |
| 336 | B |
| 337 | D |
| 338 | A |
| 339 | B |
| 340 | C |
| 341 | B |
| 342 | C |
| 343 | C |
| 344 | A |
| 353 | B |
| 354 | B |
| 355 | A |
| 356 | A |
| 357 | A |
| 373 | A |
| 388 | A |
| 389 | B |
| 390 | A |
| 391 | A |
| 392 | B |
| 394 | A |
| 402 | C |
| 407 | B |
| 408 | A |

### III. Pharmaceutical Compositions

### Example C-1: Oral Solution

To prepare a pharmaceutical composition for oral delivery, a sufficient amount of a compound disclosed herein, or a pharmaceutically acceptable salt or solvate thereof, is added to water (with optional solubilizer(s), optional buffer(s) and taste masking excipients) to provide a 20 mg/mL solution.

### Example C-2: Oral Tablet

A tablet is prepared by mixing 20-50% by weight of a compound disclosed herein, or a pharmaceutically acceptable salt or solvate thereof, 20-50% by weight of microcrystalline cellulose, 1-10% by weight of low-substituted hydroxypropyl cellulose, and 1-10% by weight of magnesium stearate or other appropriate excipients. Tablets are prepared by direct compression. The total weight of the compressed tablets is maintained at 100−500 mg.

### Example C-3: Oral Capsule

To prepare a pharmaceutical composition for oral delivery, 10-500 mg of a compound disclosed herein, or a pharmaceutically acceptable salt or solvate thereof, is mixed with starch or other suitable powder blend. The mixture is incorporated into an oral dosage unit such as a hard gelatin capsule, which is suitable for oral administration.

In another embodiment, 10-500 mg of a compound disclosed herein, or a pharmaceutically acceptable salt or solvate thereof, is placed into size 4 capsule, or size 1 capsule (hypromellose or hard gelatin) and the capsule is closed.

From the foregoing description, one skilled in the art can easily ascertain the essential characteristics of this invention, and without departing from the spirit and scope thereof, can make various changes and modifications of the invention to adapt it to various usages and conditions.

We claim:

1. A compound of Formula (I): 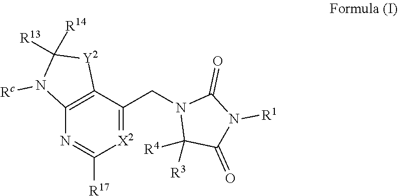
or a pharmaceutically acceptable salt thereof, wherein:
R¹ is unsubstituted or substituted phenyl, unsubstituted or substituted monocyclic heteroaryl, or unsubstituted or substituted bicyclic heteroaryl; wherein if R¹ is substituted, then it is substituted with R^{A} and 1-3 R⁵;
R^{A} is unsubstituted or substituted C₁-C₆ alkyl, unsubstituted or substituted C₁-C₆ fluoroalkyl, unsubstituted or substituted phenyl, unsubstituted or substituted 5- or 6-membered heteroaryl, unsubstituted or substituted C₃-C₆ cycloalkyl, unsubstituted or substituted C₃-C₆ cycloalkenyl, unsubstituted or substituted 3- to 6-membered heterocycloalkyl, halogen, —CN, —SR^{A1}, —S(═O)R^{A1}, —S(═O)₂R^{A1}, —SF₅, or —SiMe₃;
R^{A1} is C₁-C₆ alkyl, C₁-C₆ fluoroalkyl, C₃-C₆ cycloalkyl, or 3- to 6-membered heterocycloalkyl;
or R^{A} is 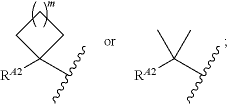
R^{A2} is halogen, —OH, —CN, C₁-C₄ alkyl, C₁-C₄ fluoroalkyl, C₁-C₄ alkoxy, C₁-C₄ fluoroalkoxyl, or C₁-C₄ hydroxyalkyl;
m is 0, 1, 2, or 3;
each R⁵ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²¹, —CO₂R²², —N(R²²)₂, —C(═O)N(R²²)₂, —NR²²C(═O)R²¹, —S(═O)₂N(R²²)₂, or —NR²²S(═O)₂R²¹;
or R¹ is 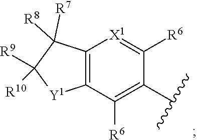
X¹ is CR⁶ or N;
each R⁶ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²¹, —CO₂R²², —N(R²²)₂, —C(═O)N(R²²)₂, —NR²²C(═O)R²¹, —S(═O)₂N(R²²)₂, or —NR²²S(═O)₂R²¹,
Y¹ is —O—, —NR¹¹—, or —SO₂—;
R¹¹ is hydrogen, C₁-C₆ alkyl, C₁-C₆ fluoroalkyl, —C(═O)NH₂, —C(═O)R¹², or —S(═O)₂R¹²;
R¹² is unsubstituted or substituted C₁-C₆ alkyl, unsubstituted or substituted C₃-C₆ cycloalkyl, or unsubstituted or substituted 3- to 6-membered heterocycloalkyl;
R⁷ and R⁸ are each independently hydrogen, —OH, C₁-C₆ alkyl, C₁-C₆ fluoroalkyl, or C₁-C₆ alkoxy;
or R⁷ and R⁸ are taken together with the carbon atom to which they are attached to form an unsubstituted or substituted C₃-C₆ cycloalkyl or unsubstituted or substituted 3- to 6-membered heterocycloalkyl;
R⁹ and R¹⁰ are each independently hydrogen, halogen, C₁-C₆ alkyl, C₁-C₆ fluoroalkyl, or C₁-C₆ alkoxy;
or R⁹ and R¹⁰ are taken together with the carbon atom to which they are attached to form a —C(═O)—;
or R⁹ and R¹⁰ are taken together with the carbon atom to which they are attached to form an unsubstituted or substituted C₃-C₆ cycloalkyl or unsubstituted or substituted 3- to 6-membered heterocycloalkyl;
R³ is hydrogen, unsubstituted or substituted C₁-C₆ alkyl, unsubstituted or substituted C₁-C₆ fluoroalkyl, unsubstituted or substituted C₁-C₆ alkoxy, unsubstituted or substituted phenyl, unsubstituted or substituted benzyl, unsubstituted or substituted monocyclic heteroaryl, unsubstituted or substituted C₃-C₆ cycloalkyl, or unsubstituted or substituted 3- to 6-membered heterocycloalkyl;
R⁴ is hydrogen or C₁-C₆ alkyl;
or R³ and R⁴ are taken together with the carbon atom to which they are attached to form a C₃-C₆ cycloalkyl or 3- to 6-membered heterocycloalkyl;
R^{c} is hydrogen, C₁-C₆ alkyl, C₁-C₆ fluoroalkyl, —C(═O)N(R²²)₂, —C(═O)R²¹, —C(═O)OR²², or —S(═O)₂R²¹;
X² is CR¹⁷ or N;
each R¹⁷ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²¹, —CO₂R²², —N(R²²)₂, —C(═O)N(R²²)₂, —OC(═O)R²¹, —NR²²C(═O)R²¹, —S(═O)₂N(R²²)₂, or —NR²²S(═O)₂R²¹;
R¹³ and R¹⁴ are each independently hydrogen, halogen, unsubstituted or substituted C₁-C₆ alkyl, C₁-C₆ fluoroalkyl, phenyl, monocyclic heteroaryl, C₃-C₆ cycloalkyl, 3- to 6-membered heterocycloalkyl, —CN, —OH, —OR²¹, —CO₂R²², —N(R²²)₂, —C(═O)N(R²²)₂, or —OC(═O)R²¹;
or R¹³ and R¹⁴ are taken together with the carbon atom to which they are attached to form a —C(═O)—;
or R¹³ and R¹⁴ are taken together with the carbon atom to which they are attached to form a C₃-C₆ cycloalkyl or 3- to 6-membered heterocycloalkyl;
Y² is —(CR¹⁵R¹⁶)ₙ—, —O—, or —O—CR¹⁵R¹⁶—;
each R¹⁵ and R¹⁶ is independently hydrogen, halogen, cyano, C₁-C₆ alkyl, C₁-C₆ fluoroalkyl, C₁-C₆ alkoxy, phenyl, monocyclic heteroaryl, C₃-C₆ cycloalkyl, or 3- to 6-membered heterocycloalkyl;
or R¹⁴ and one R¹⁶ on an adjacent carbon atom are taken together to form an alkene bond;
n is 1 or 2;
each R²¹ is independently unsubstituted or substituted C₁-C₆ alkyl, unsubstituted or substituted C₃-C₆ cycloalkyl, unsubstituted or substituted 3- to 6-membered heterocycloalkyl, unsubstituted or substituted aryl, or unsubstituted or substituted heteroaryl;
each R²² is independently hydrogen, unsubstituted or substituted C₁-C₆ alkyl, unsubstituted or substituted C₃-C₆ cycloalkyl, unsubstituted or substituted 3- to 6-membered heterocycloalkyl, unsubstituted or substituted aryl, or unsubstituted or substituted heteroaryl; or two R²² on the same N atom are taken together with the N atom to which they are attached to form an unsubstituted or substituted N-containing heterocycloalkyl;
wherein each substituted alkyl, substituted fluoroalkyl, substituted aryl, substituted heteroaryl, substituted cycloalkyl, and substituted heterocycloalkyl is substituted with one or more R¹ groups independently selected from the group consisting of halogen, C₁-C₆ alkyl, C₁-C₆ fluoroalkyl, C₃-C₆ cycloalkyl, 3- to 6-membered heterocycloalkyl, phenyl, benzyl, 5-membered heteroaryl, 6-membered heteroaryl, —CN, —OR²³, —CO₂R²³, —C(═O)N(R²³)₂, —N(R²³)₂, —NR²³C(═O)R²⁴, —SR²³, —S(═O)R²⁴, —SO₂R²⁴, or —SO₂N(R²³)₂;
each R²³ is independently selected from hydrogen, C₁-C₆ alkyl, C₃-C₆ cycloalkyl, 3- to 6-membered heterocycloalkyl, phenyl, benzyl, 5-membered heteroaryl and 6-membered heteroaryl; or two R² groups are taken together with the N atom to which they are attached to form a 3- to 6-membered N-containing heterocycloalkyl;
each R²⁴ is independently selected from C₁-C₆ alkyl, C₃-C₆ cycloalkyl, 3- to 6-membered heterocycloalkyl, phenyl, benzyl, 5-membered heteroaryl and 6-membered heteroaryl.

2. The compound of claim 1, or a pharmaceutically acceptable salt thereof, wherein:
R^{c} is hydrogen, C₁-C₆ alkyl, or —C(═O)OR²²;
X² is CR¹⁷;
each R¹⁷ is independently hydrogen, halogen, C₁-C₆ alkyl, or C₁-C₆ fluoroalkyl;
R³ and R¹⁴ are each independently hydrogen, halogen, C₁-C₆ alkyl, C₁-C₆ fluoroalkyl, C₃-C₆ cycloalkyl, or 3- to 6-membered heterocycloalkyl;
or R¹³ and R¹⁴ are taken together with the carbon atom to which they are attached to form a —C(═O)—;
or R¹³ and R¹⁴ are taken together with the carbon atom to which they are attached to form a C₃-C₄ cycloalkyl;
Y² is —(CR¹⁵R¹⁶)ₙ—, —O—, or —O—CR¹⁵R¹⁶—; and
each R⁵ and R¹⁶ is independently hydrogen, halogen, C₁-C₆ alkyl, C₁-C₆ fluoroalkyl, or C₃-C₆ cycloalkyl;
or R¹⁴ and one R¹⁶ on an adjacent carbon atom are taken together to form an alkene bond.

3. The compound of claim 1 or claim 2, or a pharmaceutically acceptable salt thereof, wherein:
R^{c} is hydrogen;
X² is CR¹⁷;
each R¹⁷ is hydrogen;
R¹³ and R¹⁴ are each hydrogen;
or R¹³ and R¹⁴ are taken together with the carbon atom to which they are attached to form a —C(═O)—; and
Y² is —(CR¹⁵R¹⁶)ₙ—, —O—, or —O—CR¹⁵R¹⁶—.

4. The compound of any one of claims 1-3, or a pharmaceutically acceptable salt thereof, wherein:
each R¹⁵ and R¹⁶ is independently hydrogen, C₁-C₆ alkyl, C₁-C₆ fluoroalkyl, or C₃-C₆ cycloalkyl.

5. The compound of any one of claims 1-4, or a pharmaceutically acceptable salt thereof, wherein: 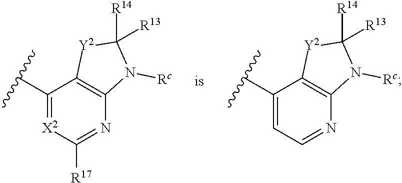
Y² is —CR¹⁵R¹⁶— or —CR¹⁵R¹⁶—CR¹⁵R¹⁶—.

6. The compound of any one of claims 1-5, or a pharmaceutically acceptable salt thereof, wherein:

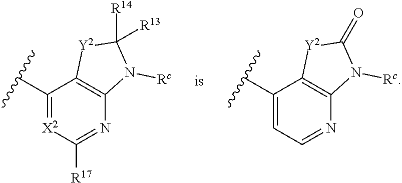

7. The compound of any one of claims 1-5, or a pharmaceutically acceptable salt thereof, wherein:
R¹⁴ and one R¹⁶ on an adjacent carbon atom are taken together to form an alkene bond.

8. The compound of any one of claims 1-7, or a pharmaceutically acceptable salt thereof, wherein:
R³ is C₁-C₆ alkyl; and
R⁴ is C₁-C₆ alkyl;
or R³ and R⁴ are taken together with the carbon atom to which they are attached to form a C₃-C₆ cycloalkyl.

9. The compound of any one of claims 1-8, or a pharmaceutically acceptable salt thereof, wherein:
R¹ is unsubstituted or substituted phenyl or unsubstituted or substituted monocyclic heteroaryl; wherein if R¹ is substituted, then it is substituted with R^{A} and 1-3 R⁵.

10. The compound of any one of claims 1-8, or a pharmaceutically acceptable salt thereof, wherein:
R¹ is 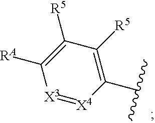
wherein
X³ and X⁴ are each independently CR⁵ or N; and
each R⁵ is independently hydrogen, halogen, C₁-C₆ alkyl, C₁-C₆ fluoroalkyl, C₃-C₆ cycloalkyl, 3- to 6-membered heterocycloalkyl, —CN, or —OR²¹.

11. The compound of claim 10, or a pharmaceutically acceptable salt thereof, wherein:
X¹ and X⁴ are each CR⁵; and
each R⁵ is hydrogen.

12. The compound of any one of claims 1-11, or a pharmaceutically acceptable salt thereof, wherein:
R^{A} is C₁-C₆ alkyl, C₁-C₆ fluoroalkyl, halogen, —CN, —SR^{A1}, —S(═O)R^{A1}, —S(═O)₂R^{A1}, —SF₅, or —SiMe₃;
R^{A1} is C₁-C₄ alkyl, or C₁-C₄ fluoroalkyl;
or R^{A} is 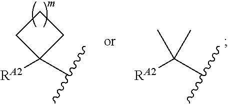
R^{A2} is halogen, —CN, C₁-C₄ alkyl, or C₁-C₄ fluoroalkyl; and
m is 0 or 1.

13. The compound of any one of claims 1-12, or a pharmaceutically acceptable salt thereof, wherein:
R^{A} is —CH₃, —CH₂CH₃, —CH(CH₃)₂, —C(CH₃)₃—CF₃, —F, —Cl, —Br, —CN, —SCF₃, —S(═O)CF₃, —S(═O)₂CF₃, —S(═O)₂CHF₂, —S(═O)₂C(CH₃)₃, —S(═O)₂-(cyclopropyl), —SF₅, or —SiMe₃;
or R^{A} is

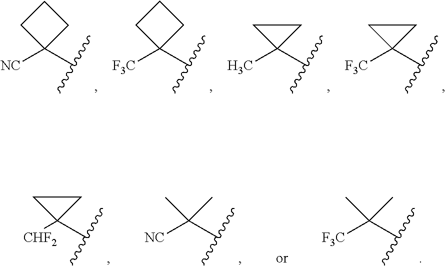

14. The compound of any one of claims 1-8, or a pharmaceutically acceptable salt thereof, wherein: R¹ is 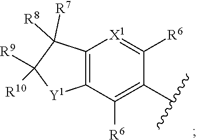
X¹ is CR⁶ or N; and
each R⁶ is independently hydrogen, halogen, C₁-C₆ alkyl, C₁-C₆ fluoroalkyl, unsubstituted or substituted C₃-C₆ cycloalkyl, unsubstituted or substituted 3- to 6-membered heterocycloalkyl, —CN, —OH, —OR²¹, —CO₂R²², —N(R²²)₂, —C(═O)N(R²²)₂, —NR²²C(═O)R²¹, —S(═O)₂N(R²²)₂, or —NR²²S(═O)₂R²¹.

15. The compound of claim 14, or a pharmaceutically acceptable salt, wherein:
Y¹ is —NR¹¹—;
R¹¹ is hydrogen, C₁-C₆ alkyl, C₁-C₆ fluoroalkyl, —C(═O)NH₂, —C(═O)R¹², or —S(═O)₂R¹²; and
R¹² is unsubstituted or substituted C₁-C₆ alkyl or unsubstituted or substituted C₃-C₆ cycloalkyl.

16. The compound of claim 14 or claim 15, or a pharmaceutically acceptable salt thereof, wherein:
R⁷ and R⁸ are each independently hydrogen, —OH, C₁-C₆ alkyl, C₁-C₆ fluoroalkyl, or C₁-C₆ alkoxy;
or R⁷ and R⁸ are taken together with the carbon atom to which they are attached to form an unsubstituted or substituted C₃-C₄ cycloalkyl;
R⁹ and R¹⁰ are each independently hydrogen, halogen, C₁-C₆ alkyl, C₁-C₆ fluoroalkyl, or C₁-C₆ alkoxy;
or R⁹ and R¹⁰ are taken together with the carbon atom to which they are attached to form a —C(═O)—;
or R⁹ and R¹⁰ are taken together with the carbon atom to which they are attached to form an unsubstituted or substituted C₃-C₄ cycloalkyl.

17. A compound of Formula (II): 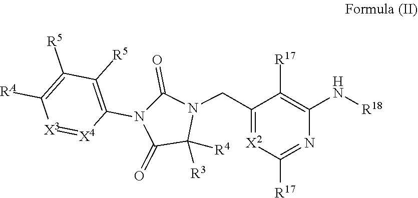
or a pharmaceutically acceptable salt thereof, wherein:
R^{A} is unsubstituted or substituted C₁-C₆ fluoroalkyl, unsubstituted or substituted phenyl, unsubstituted or substituted 5- or 6-membered heteroaryl, unsubstituted or substituted C₃-C₆ cycloalkyl, unsubstituted or substituted C₃-C₆ cycloalkenyl, or unsubstituted or substituted 3- to 6-membered heterocycloalkyl, halogen, —CN, —SF₅, or —SiMe₃;
or R^{A} is 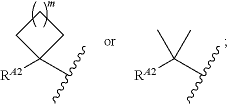
R^{A2} is halogen, —OH, —CN, C₁-C₄ alkyl, C₁-C₄ fluoroalkyl, C₁-C₄ alkoxy, C₁-C₄ fluoroalkoxyl, or C₁-C₄ hydroxyalkyl;
m is 0, 1, 2, or 3;
X³ and X⁴ are each independently CR⁵ or N;
R³ is hydrogen, unsubstituted or substituted C₁-C₆ alkyl, unsubstituted or substituted C₁-C₆ fluoroalkyl, unsubstituted or substituted C₁-C₆ alkoxy, unsubstituted or substituted phenyl, unsubstituted or substituted benzyl, unsubstituted or substituted monocyclic heteroaryl, unsubstituted or substituted C₃-C₆ cycloalkyl, or unsubstituted or substituted 3- to 6-membered heterocycloalkyl;
R⁴ is hydrogen or C₁-C₆ alkyl;
or R³ and R⁴ are taken together with the carbon atom to which they are attached to form a C₃-C₆ cycloalkyl or 3- to 6-membered heterocycloalkyl;
each R is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²¹, —CO₂R²², —N(R²²)₂, —C(═O)N(R²²)₂, —NR²²C(═O)R²¹, —S(═O)₂N(R²²)₂, or —NR²²S(═O)₂R²¹;
X² is CR¹⁷ or N;
each R¹⁷ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²¹, —CO₂R²², —N(R²²)₂, —C(═O)N(R²²)₂, —NR²²C(═O)R²¹, —S(═O)₂N(R²²)₂, or —NR²²S(═O)₂R²¹;
R¹⁸ is hydrogen, unsubstituted or substituted C₁-C₆ alkyl, unsubstituted or substituted C₁-C₆ fluoroalkyl, unsubstituted or substituted C₁-C₆ heteroalkyl, unsubstituted or substituted C₁-C₆ alkenyl, unsubstituted or substituted C₁-C₆ alkynyl, unsubstituted or substituted carbocycle, unsubstituted or substituted heterocycle, —CHR²⁰-(unsubstituted or substituted carbocycle), —CHR²⁰-(unsubstituted or substituted heterocycle), —C(═O)R¹⁹, —CO₂R¹⁹, or —C(═O)NHR¹⁹;
R¹⁹ is hydrogen, unsubstituted or substituted C₁-C₆ alkyl, unsubstituted or substituted C₃-C₁₀ cycloalkyl, unsubstituted or substituted 3- to 10-membered heterocycloalkyl;
R²⁰ is hydrogen or —CH₃;
each R²¹ is independently unsubstituted or substituted C₁-C₆ alkyl, unsubstituted or substituted C₃-C₆ cycloalkyl, unsubstituted or substituted 3- to 6-membered heterocycloalkyl, unsubstituted or substituted aryl, or unsubstituted or substituted heteroaryl;
each R²² is independently hydrogen, unsubstituted or substituted C₁-C₆ alkyl, unsubstituted or substituted C₃-C₆ cycloalkyl, unsubstituted or substituted 3- to 6-membered heterocycloalkyl, unsubstituted or substituted aryl, or unsubstituted or substituted heteroaryl; or two R²² on the same N atom are taken together with the N atom to which they are attached to form an unsubstituted or substituted N-containing heterocycloalkyl;
wherein each substituted alkyl, substituted fluoroalkyl, substituted aryl, substituted heteroaryl, substituted cycloalkyl, and substituted heterocycloalkyl is substituted with one or more R¹ groups independently selected from the group consisting of halogen, C₁-C₆ alkyl, C₁-C₆ fluoroalkyl, C₃-C₆ cycloalkyl, 3- to 6-membered heterocycloalkyl, phenyl, benzyl, 5-membered heteroaryl, 6-membered heteroaryl, —CN, —OR²³, —CO₂R³, —C(═O)N(R²³)₂, —N(R²³)₂, —NR²³C(═O)R²⁴, —SR²³, —S(═O)R²⁴, —SO₂R²⁴, or —SO₂N(R²³)₂;
each R²³ is independently selected from hydrogen, C₁-C₆ alkyl, C₃-C₆ cycloalkyl, 3- to 6-membered heterocycloalkyl, phenyl, benzyl, 5-membered heteroaryl and 6-membered heteroaryl; or two R³ groups are taken together with the N atom to which they are attached to form a 3- to 6-membered N-containing heterocycloalkyl;
each R²⁴ is independently selected from C₁-C₆ alkyl, C₃-C₆ cycloalkyl, 3- to 6-membered heterocycloalkyl, phenyl, benzyl, 5-membered heteroaryl and 6-membered heteroaryl.

18. The compound of claim 17, or a pharmaceutically acceptable salt thereof, wherein:
R³ is C₁-C₆ alkyl; and
R⁴ is C₁-C₆ alkyl;
or R³ and R⁴ are taken together with the carbon atom to which they are attached to form a C₃-C₆ cycloalkyl.

19. The compound of claim 17 or claim 18, or a pharmaceutically acceptable salt thereof, wherein:
each R⁵ is independently hydrogen, halogen, C₁-C₆ alkyl, C₁-C₆ fluoroalkyl, C₃-C₆ cycloalkyl, 3- to 6-membered heterocycloalkyl, —CN, or —OR²¹.

20. The compound of any one of claims 17-19, or a pharmaceutically acceptable salt thereof, wherein:
X³ and X⁴ are each CR⁵; and
each R⁵ is hydrogen.

21. The compound of any one of claims 17-20, or a pharmaceutically acceptable salt thereof, wherein:
R^{A} is C₁-C₄ fluoroalkyl, halogen, —CN, —SF₅, or —SiMe₃;
or R^{A} is 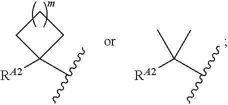
R^{A2} is halogen, —CN, C₁-C₄ alkyl, or C₁-C₄ fluoroalkyl; and
m is 1.

22. The compound of any one of claims 17-21, or a pharmaceutically acceptable salt thereof, wherein:
R^{A} is —CF₃, —F, —C₁, —Br, —CN, —SCF₃, —SF₅, or —SiMe₃;
or R^{A} is

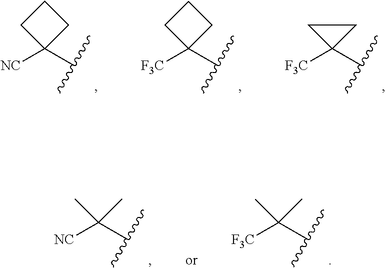

23. The compound of any one of claims 17-22, or a pharmaceutically acceptable salt thereof, wherein:
X² is CR¹⁷; and
each R¹⁷ is independently hydrogen, halogen, C₁-C₆ alkyl, or C₁-C₆ fluoroalkyl.

24. The compound of any one of claims 17-23, or a pharmaceutically acceptable salt thereof, wherein:
X² is CR¹⁷; and
each R¹⁷ is hydrogen.

25. The compound of any one of claims 17-24, or a pharmaceutically acceptable salt thereof, wherein:
R¹⁸ is hydrogen, unsubstituted or substituted C₁-C₆ alkyl, unsubstituted or substituted C₁-C₆ fluoroalkyl, unsubstituted or substituted C₁-C₆ heteroalkyl, unsubstituted or substituted C₁-C₆ alkenyl, unsubstituted or substituted C₁-C₆ alkynyl.

26. The compound of claim 25, or a pharmaceutically acceptable salt thereof, wherein:
R¹⁸ is substituted C₁-C₆ alkyl, unsubstituted or substituted C₁-C₆ fluoroalkyl, unsubstituted or substituted C₁-C₆ heteroalkyl.

27. The compound of claim 26, or a pharmaceutically acceptable salt thereof, wherein:
R¹⁸ is C₁-C₆ alkyl which is substituted by —CN, —OH, or —OMe.

28. The compound of any one of claims 17-24, or a pharmaceutically acceptable salt thereof, wherein:
R¹⁸ is unsubstituted or substituted C₃-C₁₀ cycloalkyl, unsubstituted or substituted 3- to 10-membered heterocycloalkyl, unsubstituted or substituted aryl, or unsubstituted or substituted heteroaryl.

29. The compound of claim 28, or a pharmaceutically acceptable salt thereof, wherein:
R¹⁸ is unsubstituted or substituted C₃-C₆ monocyclic cycloalkyl, unsubstituted or substituted C₅-C₁₀ bicyclic cycloalkyl, or unsubstituted or substituted 3- to 10-membered monocyclic or bicyclic heterocycloalkyl.

30. The compound of claim 29, or a pharmaceutically acceptable salt thereof, wherein:
R¹⁸ is unsubstituted or substituted 3- to 10-membered monocyclic or bicyclic heterocycloalkyl containing an oxygen atom;
or R¹⁸ is unsubstituted or substituted 5- to 10-membered bicyclic heterocycloalkyl which is a bridged, fused, or spirocyclic heterocycloalkyl.

31. The compound of any one of claims 17-24, or a pharmaceutically acceptable salt thereof, wherein:
R¹⁸ is —CHR²⁰-(unsubstituted or substituted C₃-C₆ cycloalkyl), —CHR²⁰-(unsubstituted or substituted 3- to 6-membered heterocycloalkyl), —CHR²⁰-(unsubstituted or substituted phenyl), or —CHR²⁰-(unsubstituted or substituted 5- or 6-membered heteroaryl); and
R²⁰ is hydrogen or —CH₃.

32. The compound of any one of claims 17-24, or a pharmaceutically acceptable salt thereof, wherein:
R¹⁸ is —C(═O)R¹⁹, —CO₂R¹⁹, or —C(═O)NHR¹⁹; and
R¹⁹ is unsubstituted or substituted C₁-C₆ alkyl, unsubstituted or substituted C₃-C₁₀ cycloalkyl, unsubstituted or substituted 3- to 10-membered heterocycloalkyl.

33. A compound of Formula (III): 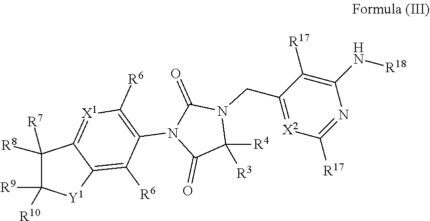
or a pharmaceutically acceptable salt thereof, wherein:
X¹ is CR⁶ or N;
each R⁶ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²¹, —CO₂R²², —N(R²²)₂, —C(═O)N(R²²)₂, —NR²²C(═O)R²¹, —S(═O)₂N(R²²)₂, or —NR²²S(═O)₂R²¹;
Y¹ is —O—, —NR¹¹—, or —SO₂—;
R¹¹ is hydrogen, C₁-C₆ alkyl, C₁-C₆ fluoroalkyl, —C(═O)NH₂, —C(═O)R¹², or —S(═O)₂R¹²,
R¹² is unsubstituted or substituted C₁-C₆ alkyl, unsubstituted or substituted C₃-C₆ cycloalkyl, or unsubstituted or substituted 3- to 6-membered heterocycloalkyl;
R⁷ and R⁸ are each independently hydrogen, —OH, C₁-C₆ alkyl, C₁-C₆ fluoroalkyl, or C₁-C₆ alkoxy;
or R⁷ and R⁸ are taken together with the carbon atom to which they are attached to form an unsubstituted or substituted C₃-C₆ cycloalkyl or unsubstituted or substituted 3- to 6-membered heterocycloalkyl;
R⁹ and R¹⁰ are each independently hydrogen, halogen, C₁-C₆ alkyl, C₁-C₆ fluoroalkyl, or C₁-C₆ alkoxy;
or R⁹ and R¹⁰ are taken together with the carbon atom to which they are attached to form a —C(═O)—;
or R⁹ and R¹⁰ are taken together with the carbon atom to which they are attached to form an unsubstituted or substituted C₃-C₆ cycloalkyl or unsubstituted or substituted 3- to 6-membered heterocycloalkyl;
R³ is hydrogen, unsubstituted or substituted C₁-C₆ alkyl, unsubstituted or substituted C₁-C₆ fluoroalkyl, unsubstituted or substituted C₁-C₆ alkoxy, unsubstituted or substituted phenyl, unsubstituted or substituted benzyl, unsubstituted or substituted monocyclic heteroaryl, unsubstituted or substituted C₃-C₆ cycloalkyl, or unsubstituted or substituted 3- to 6-membered heterocycloalkyl;
R⁴ is hydrogen or C₁-C₆ alkyl,
or R³ and R⁴ are taken together with the carbon atom to which they are attached to form a C₁-C₆ cycloalkyl or 3- to 6-membered heterocycloalkyl;
X² is CR¹⁷ or N;
each R¹⁷ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²², —CO₂R²², —N(R²²)₂, —C(═O)N(R²²)₂, —NR²²C(═O)R²¹, —S(═O)₂N(R²²)₂, or —NR²²S(═O)₂R²¹;
R¹⁸ is unsubstituted or substituted C₁-C₆ alkyl, unsubstituted or substituted C₁-C₆ fluoroalkyl, unsubstituted or substituted C₁-C₆ heteroalkyl, unsubstituted or substituted C₁-C₆ alkenyl, unsubstituted or substituted C₁-C₆ alkynyl, unsubstituted or substituted C₃-C₁₀ cycloalkyl, unsubstituted or substituted 3- to 10-membered heterocycloalkyl, —CHR²⁰-(unsubstituted or substituted carbocycle), —CHR²⁰-(unsubstituted or substituted heterocycle), —C(═O)R¹⁹, —CO₂R¹⁹, or —C(═O)NHR¹⁹;
R¹⁹ is hydrogen, unsubstituted or substituted C₁-C₆ alkyl, unsubstituted or substituted C₃-C₁₀ cycloalkyl, or unsubstituted or substituted 3- to 10-membered heterocycloalkyl;
R²⁰ is hydrogen or —CH₃;
each R²¹ is independently unsubstituted or substituted C₁-C₆ alkyl, unsubstituted or substituted C₃-C₆ cycloalkyl, unsubstituted or substituted 3- to 6-membered heterocycloalkyl, unsubstituted or substituted aryl, or unsubstituted or substituted heteroaryl;
each R²² is independently hydrogen, unsubstituted or substituted C₁-C₆ alkyl, unsubstituted or substituted C₃-C₆ cycloalkyl, unsubstituted or substituted 3- to 6-membered heterocycloalkyl, unsubstituted or substituted aryl, or unsubstituted or substituted heteroaryl; or two R²² on the same N atom are taken together with the N atom to which they are attached to form an unsubstituted or substituted N-containing heterocycloalkyl;
wherein each substituted alkyl, substituted fluoroalkyl, substituted aryl, substituted heteroaryl, substituted cycloalkyl, and substituted heterocycloalkyl is substituted with one or more R^{Y} groups independently selected from the group consisting of halogen, C₁-C₆ alkyl, C₁-C₆ fluoroalkyl, C₃-C₆ cycloalkyl, 3- to 6-membered heterocycloalkyl, phenyl, benzyl, 5-membered heteroaryl, 6-membered heteroaryl, —CN, —OR²³, —CO₂R²³, —C(═O)N(R²³)₂, —N(R²³)₂, —NR²³C(═O)R²⁴, —SR²³, —S(═O)R²⁴, —SO₂R²⁴, or —SO₂N(R²³)₂;
each R²³ is independently selected from hydrogen, C₁-C₆ alkyl, C₃-C₆ cycloalkyl, 3- to 6-membered heterocycloalkyl, phenyl, benzyl, 5-membered heteroaryl and 6-membered heteroaryl; or two R²³ groups are taken together with the N atom to which they are attached to form a 3- to 6-membered N-containing heterocycloalkyl;
each R²⁴ is independently selected from C₁-C₆ alkyl, C₃-C₆ cycloalkyl, 3- to 6-membered heterocycloalkyl, phenyl, benzyl, 5-membered heteroaryl and 6-membered heteroaryl.

34. The compound of claim 33, or a pharmaceutically acceptable salt thereof, wherein:
R³ is C₁-C₆ alkyl; and
R⁴ is C₁-C₆ alkyl;
or R³ and R⁴ are taken together with the carbon atom to which they are attached to form a C₃-C₆ cycloalkyl.

35. The compound of claim 33 or claim 34, or a pharmaceutically acceptable salt thereof, wherein:
X is CR⁶ or N; and
each R⁶ is independently hydrogen, halogen, C₁-C₆ alkyl, C₁-C₆ fluoroalkyl, unsubstituted or substituted C₃-C₆ cycloalkyl, unsubstituted or substituted 3- to 6-membered heterocycloalkyl, —CN, —OH, —OR²¹, —CO₂R²², —N(R²²)₂, —C(═O)N(R²²)₂, —NR²²C(═O)R²¹, —S(═O)₂N(R²²)₂, or —NR²²S(═O)₂R²¹.

36. The compound of any one of claims 33-35, or a pharmaceutically acceptable salt thereof, wherein:
Y¹ is —NR¹¹—;
R¹¹ is hydrogen, C₁-C₆ alkyl, C₁-C₆ fluoroalkyl, —C(═O)NH₂, —C(═O)R¹², or —S(═O)₂ and
R¹² is unsubstituted or substituted C₁-C₆ alkyl or unsubstituted or substituted C₃-C₆ cycloalkyl.

37. The compound of any one of claims 33-36, or a pharmaceutically acceptable salt thereof, wherein:
R⁷ and R⁸ are each independently hydrogen, —OH, C₁-C₆ alkyl, C₁-C₆ fluoroalkyl, or C₁-C₆ alkoxy;
or R⁷ and R⁸ are taken together with the carbon atom to which they are attached to form an unsubstituted or substituted C₃-C₄ cycloalkyl;
R⁹ and R¹⁰ are each independently hydrogen, halogen, C₁-C₆ alkyl, C₁-C₆ fluoroalkyl, or C₁-C₆ alkoxy;
or R⁹ and R¹⁰ are taken together with the carbon atom to which they are attached to form a —C(═O)—;
or R⁹ and R¹⁰ are taken together with the carbon atom to which they are attached to form an unsubstituted or substituted C₃-C₄ cycloalkyl.

38. The compound of any one of claims 33-37, or a pharmaceutically acceptable salt thereof, wherein:
X² is CR¹⁷; and
each R¹⁷ is independently hydrogen, halogen, C₁-C₆ alkyl, or C₁-C₆ fluoroalkyl.

39. The compound of any one of claims 33-38, or a pharmaceutically acceptable salt thereof, wherein:
X² is CR¹⁷; and
each R¹⁷ is hydrogen.

40. The compound of any one of claims 33-39, or a pharmaceutically acceptable salt thereof, wherein:
R¹⁸ is unsubstituted or substituted C₁-C₆ alkyl, unsubstituted or substituted C₁-C₆ fluoroalkyl, unsubstituted or substituted C₁-C₆ heteroalkyl, unsubstituted or substituted C₁-C₆ alkenyl, unsubstituted or substituted C₁-C₆ alkynyl.

41. The compound of claim 40, or a pharmaceutically acceptable salt thereof, wherein:
R¹⁸ is substituted C₁-C₆ alkyl, unsubstituted or substituted C₁-C₆ fluoroalkyl, unsubstituted or substituted C₁-C₆ heteroalkyl.

42. The compound of claim 41, or a pharmaceutically acceptable salt thereof, wherein:
R¹⁸ is C₁-C₆ alkyl which is substituted by —CN, —OH, or —OMe.

43. The compound of any one of claims 33-39, or a pharmaceutically acceptable salt thereof, wherein:
R¹⁸ is unsubstituted or substituted C₃-C₁₀ cycloalkyl or unsubstituted or substituted 3- to 10-membered heterocycloalkyl.

44. The compound of claim 43, or a pharmaceutically acceptable salt thereof, wherein:
R¹⁸ is unsubstituted or substituted C₃-C₆ monocyclic cycloalkyl, unsubstituted or substituted C₅-C₁₀ bicyclic cycloalkyl, or unsubstituted or substituted 3- to 10-membered monocyclic or bicyclic heterocycloalkyl.

45. The compound of claim 44, or a pharmaceutically acceptable salt thereof, wherein:
R¹⁸ is unsubstituted or substituted 3- to 10-membered monocyclic or bicyclic heterocycloalkyl containing an oxygen atom;
or R¹⁸ is unsubstituted or substituted 5- to 10-membered bicyclic heterocycloalkyl which is a bridged, fused, or spirocyclic heterocycloalkyl.

46. The compound of any one of claims 33-39, or a pharmaceutically acceptable salt thereof, wherein:
R¹⁸ is —CHR²⁰-(unsubstituted or substituted C₃-C₆ cycloalkyl), —CHR²⁰-(unsubstituted or substituted 3- to 6-membered heterocycloalkyl), —CHR²⁰-(unsubstituted or substituted phenyl), or —CHR²⁰-(unsubstituted or substituted 5- or 6-membered heteroaryl); and
R²⁰ is hydrogen or —CH₃.

47. The compound of any one of claims 33-39, or a pharmaceutically acceptable salt thereof, wherein:
R¹⁸ is —C(═O)R¹⁹, —CO₂R¹⁹, or —C(═O)NHR¹⁹; and
R¹⁹ is unsubstituted or substituted C₁-C₆ alkyl, unsubstituted or substituted C₃-C₁₀ cycloalkyl, unsubstituted or substituted 3- to 10-membered heterocycloalkyl.

48. A compound of Formula (IV): 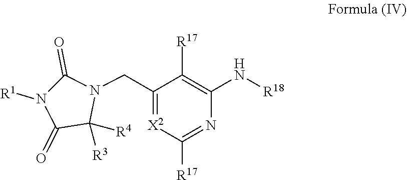
or a pharmaceutically acceptable salt thereof, wherein:
R¹ is unsubstituted or substituted bicyclic heteroaryl; wherein if R¹ is substituted, then it is substituted with R^{A} and 1-3 R⁵;
R^{A} is unsubstituted or substituted C₁-C₆ alkyl, unsubstituted or substituted C₁-C₆ fluoroalkyl, unsubstituted or substituted C₃-C₆ cycloalkyl, unsubstituted or substituted 3- to 6-membered heterocycloalkyl, halogen, —CN, —SR^{A1}, —S(═O)R^{A1}, —S(═O)₂R^{A1}, —OR^{A1}, —SF₅, or —SiMe₃;
R^{A1} is C₁-C₆ alkyl, C₁-C₆ fluoroalkyl, C₃-C₆ cycloalkyl, or 3- to 6-membered heterocycloalkyl;
each R⁵ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²¹, —CO₂R²², —N(R²²)₂, —C(═O)N(R²²)₂, —NR²²C(═O)R²¹, —S(═O)₂N(R²²)₂, or —NR²²S(═O)₂R²¹;
R³ is hydrogen, unsubstituted or substituted C₁-C₆ alkyl, unsubstituted or substituted C₁-C₆ fluoroalkyl, unsubstituted or substituted C₁-C₆ alkoxy, unsubstituted or substituted phenyl, unsubstituted or substituted benzyl, unsubstituted or substituted monocyclic heteroaryl, unsubstituted or substituted C₃-C₆ cycloalkyl, or unsubstituted or substituted 3- to 6-membered heterocycloalkyl;
R⁴ is hydrogen or C₁-C₆ alkyl;
or R³ and R⁴ are taken together with the carbon atom to which they are attached to form a C₃-C₆ cycloalkyl or 3- to 6-membered heterocycloalkyl;
X² is CR¹⁷ or N;
each R¹⁷ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²¹, —CO₂R²², —N(R²²)₂, —C(═O)N(R²²)₂, —NR²²C(═O)R²¹, —S(═O)₂N(R²²)₂, or —NR²²S(═O)₂R²¹;
R¹⁸ is hydrogen, unsubstituted or substituted C₁-C₆ alkyl, unsubstituted or substituted C₁-C₆ fluoroalkyl, unsubstituted or substituted C₁-C₆ heteroalkyl, unsubstituted or substituted C₁-C₆ alkenyl, unsubstituted or substituted C₁-C₆ alkynyl, unsubstituted or substituted carbocycle, unsubstituted or substituted heterocycle, —CHR²⁰-(unsubstituted or substituted carbocycle), —CHR²⁰-(unsubstituted or substituted heterocycle), —C(═O)R¹⁹, —CO₂R¹⁹, or —C(═O)NHR¹⁹;
R¹⁹ is hydrogen, unsubstituted or substituted C₁-C₆ alkyl, unsubstituted or substituted C₃-C₁₀ cycloalkyl, or unsubstituted or substituted 3- to 10-membered heterocycloalkyl;
R²⁰ is hydrogen or —CH₃;
each R²¹ is independently unsubstituted or substituted C₁-C₆ alkyl, unsubstituted or substituted C₃-C₆ cycloalkyl, unsubstituted or substituted 3- to 6-membered heterocycloalkyl, unsubstituted or substituted aryl, or unsubstituted or substituted heteroaryl;
each R²² is independently hydrogen, unsubstituted or substituted C₁-C₆ alkyl, unsubstituted or substituted C₃-C₆ cycloalkyl, unsubstituted or substituted 3- to 6-membered heterocycloalkyl, unsubstituted or substituted aryl, or unsubstituted or substituted heteroaryl; or two R²² on the same N atom are taken together with the N atom to which they are attached to form an unsubstituted or substituted N-containing heterocycloalkyl;
wherein each substituted alkyl, substituted fluoroalkyl, substituted aryl, substituted heteroaryl, substituted cycloalkyl, and substituted heterocycloalkyl is substituted with one or more R groups independently selected from the group consisting of halogen, C₁-C₆ alkyl, C₁-C₆ fluoroalkyl, C₃-C₆ cycloalkyl, 3- to 6-membered heterocycloalkyl, phenyl, benzyl, 5-membered heteroaryl, 6-membered heteroaryl, —CN, —OR²³, —CO₂R²³, —C(═O)N(R²³)₂, —N(R²³)₂, —NR²³C(═O)R²⁴, —SR²³, —S(═O)R²⁴, —SO₂R²⁴, or —SO₂N(R²³)₂;
each R²³ is independently selected from hydrogen, C₁-C₆ alkyl, C₃-C₆ cycloalkyl, 3- to 6-membered heterocycloalkyl, phenyl, benzyl, 5-membered heteroaryl and 6-membered heteroaryl; or two R³ groups are taken together with the N atom to which they are attached to form a 3- to 6-membered N-containing heterocycloalkyl;
each R²⁴ is independently selected from C₁-C₆ alkyl, C₃-C₆ cycloalkyl, 3- to 6-membered heterocycloalkyl, phenyl, benzyl, 5-membered heteroaryl and 6-membered heteroaryl.

49. The compound of claim 48, or a pharmaceutically acceptable salt thereof, wherein:
R³ is C₁-C₆ alkyl; and
R⁴ is C₁-C₆ alkyl;
or R³ and R⁴ are taken together with the carbon atom to which they are attached to form a C₃-C₆ cycloalkyl.

50. The compound of claim 48 or claim 49, or a pharmaceutically acceptable salt thereof, wherein:
R^{A} is C₁-C₄ alkyl, C₁-C₄ fluoroalkyl, C₃-C₆ cycloalkyl, 3- to 6-membered heterocycloalkyl, halogen, —CN, —SR^{A1}, —S(═O)R^{A1}, —S(═O)₂R^{A1}, —OR^{A1}, —SF₅, or —SiMe₃;
R^{A1} is C₁-C₆ alkyl, C₁-C₆ fluoroalkyl, C₃-C₆ cycloalkyl, or 3- to 6-membered heterocycloalkyl; and
each R⁵ is independently hydrogen, halogen, C₁-C₆ alkyl, C₁-C₆ fluoroalkyl, C₃-C₆ cycloalkyl, 3- to 6-membered heterocycloalkyl, —CN, or —OR²¹.

51. The compound of any one of claims 48-50, or a pharmaceutically acceptable salt thereof, wherein:
R^{A} is C₁-C₄ alkyl, C₁-C₄ fluoroalkyl, or C₃-C₆ cycloalkyl; and
each R⁵ is independently hydrogen or C₁-C₆ alkyl.

52. The compound of any one of claims 48-51, or a pharmaceutically acceptable salt thereof, wherein:
X² is CR⁷; and
each R¹⁷ is hydrogen.

53. The compound of any one of claims 48-52, or a pharmaceutically acceptable salt thereof, wherein:
R¹⁸ is hydrogen, unsubstituted or substituted C₁-C₆ alkyl, unsubstituted or substituted C₁-C₆ fluoroalkyl, unsubstituted or substituted C₁-C₆ heteroalkyl, unsubstituted or substituted C₁-C₆ alkenyl, unsubstituted or substituted C₁-C₆ alkynyl;
or R¹⁸ is substituted C₁-C₆ alkyl, unsubstituted or substituted C₁-C₆ fluoroalkyl, unsubstituted or substituted C₁-C₆ heteroalkyl;
or R¹⁸ is C₁-C₆ alkyl which is substituted by —CN, —OH, or —OMe;
or R¹⁸ is unsubstituted or substituted C₃-C₁₀ cycloalkyl, unsubstituted or substituted 3- to 10-membered heterocycloalkyl, unsubstituted or substituted aryl, or unsubstituted or substituted heteroaryl;
or R¹⁸ is unsubstituted or substituted C₃-C₆ monocyclic cycloalkyl, unsubstituted or substituted C₅-C₁₀ bicyclic cycloalkyl, or unsubstituted or substituted 3- to 10-membered monocyclic or bicyclic heterocycloalkyl;
or R¹⁸ is unsubstituted or substituted 3- to 10-membered monocyclic or bicyclic heterocycloalkyl containing an oxygen atom;
or R¹⁸ is unsubstituted or substituted 5- to 10-membered bicyclic heterocycloalkyl which is a bridged, fused, or spirocyclic heterocycloalkyl;
or R¹⁸ is —CHR²⁰-(unsubstituted or substituted C₃-C₆ cycloalkyl), —CHR²⁰-(unsubstituted or substituted 3- to 6-membered heterocycloalkyl), —CHR²⁰-(unsubstituted or substituted phenyl), or —CHR²⁰-(unsubstituted or substituted 5- or 6-membered heteroaryl); and R²⁰ is hydrogen or —CH₃;
or R¹⁸ is —C(═O)R¹⁹, —CO₂R¹⁹, or —C(═O)NHR¹⁹; and R¹⁹ is unsubstituted or substituted C₁-C₆ alkyl, unsubstituted or substituted C₃-C₁₀ cycloalkyl, unsubstituted or substituted 3- to 10-membered heterocycloalkyl.

54. A compound of Formula (V): 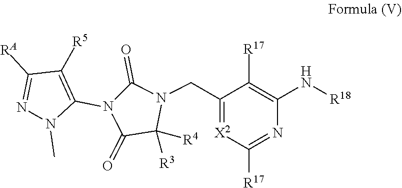
or a pharmaceutically acceptable salt thereof, wherein:
R^{A} is unsubstituted or substituted C₁-C₆ alkyl, unsubstituted or substituted C₁-C₆ fluoroalkyl, unsubstituted or substituted C₃-C₆ cycloalkyl, unsubstituted or substituted 3- to 6-membered heterocycloalkyl, halogen, —CN, —SR^{A1}, —S(═O)R^{A1}, —S(═O)₂R^{A1}, —OR^{A1}, —SF₅, or —SiMe₃;
R^{A1} is C₁-C₆ alkyl, C₁-C₆ fluoroalkyl, C₃-C₆ cycloalkyl, or 3- to 6-membered heterocycloalkyl;
R³ is hydrogen, unsubstituted or substituted C₁-C₆ alkyl, unsubstituted or substituted C₁-C₆ fluoroalkyl, unsubstituted or substituted C₁-C₆ alkoxy, unsubstituted or substituted phenyl, unsubstituted or substituted benzyl, unsubstituted or substituted monocyclic heteroaryl, unsubstituted or substituted C₃-C₆ cycloalkyl, or unsubstituted or substituted 3- to 6-membered heterocycloalkyl;
R⁴ is hydrogen or C₁-C₆ alkyl;
or R³ and R⁴ are taken together with the carbon atom to which they are attached to form a C₃-C₆ cycloalkyl or 3- to 6-membered heterocycloalkyl;
R⁵ is hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²¹, —CO₂R²², —N(R²²)₂, —C(═O)N(R²²)₂, —NR²²C(═O)R²¹, —S(═O)₂N(R²²)₂, or —NR²²S(═O)₂R²¹;
X² is CR¹⁷ or N;
each R¹⁷ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²¹, —CO₂R²², —N(R²²)₂, —C(═O)N(R²²)₂, —NR²²C(═O)R²¹, —S(═O)₂N(R²²)₂, or —NR²²S(═O)₂R²¹;
R¹⁸ is hydrogen, unsubstituted or substituted C₁-C₆ alkyl, unsubstituted or substituted C₁-C₆ fluoroalkyl, unsubstituted or substituted C₁-C₆ heteroalkyl, unsubstituted or substituted C₁-C₆ alkenyl, unsubstituted or substituted C₁-C₆ alkynyl, unsubstituted or substituted carbocycle, unsubstituted or substituted heterocycle, —CHR²⁰-(unsubstituted or substituted carbocycle), —CHR²⁰-(unsubstituted or substituted heterocycle), —C(═O)R¹⁹, —CO₂R¹⁹, or —C(═O)NHR¹⁹;
R¹⁹ is hydrogen, unsubstituted or substituted C₁-C₆ alkyl, unsubstituted or substituted C₃-C₁₀ cycloalkyl, or unsubstituted or substituted 3- to 10-membered heterocycloalkyl;
R²⁰ is hydrogen or —CH₃;
each R²¹ is independently unsubstituted or substituted C₁-C₆ alkyl, unsubstituted or substituted C₃-C₆ cycloalkyl, unsubstituted or substituted 3- to 6-membered heterocycloalkyl, unsubstituted or substituted aryl, or unsubstituted or substituted heteroaryl;
each R²² is independently hydrogen, unsubstituted or substituted C₁-C₆ alkyl, unsubstituted or substituted C₃-C₆ cycloalkyl, unsubstituted or substituted 3- to 6-membered heterocycloalkyl, unsubstituted or substituted aryl, or unsubstituted or substituted heteroaryl; or two R²² on the same N atom are taken together with the N atom to which they are attached to form an unsubstituted or substituted N-containing heterocycloalkyl;
wherein each substituted alkyl, substituted fluoroalkyl, substituted aryl, substituted heteroaryl, substituted cycloalkyl, and substituted heterocycloalkyl is substituted with one or more R groups independently selected from the group consisting of halogen, C₁-C₆ alkyl, C₁-C₆ fluoroalkyl, C₃-C₆ cycloalkyl, 3- to 6-membered heterocycloalkyl, phenyl, benzyl, 5-membered heteroaryl, 6-membered heteroaryl, —CN, —OR²², —CO₂R²³, —C(═O)N(R²³)₂, —N(R²³)₂, —NR²³C(═O)R²⁴, —SR²³, —S(═O)R²⁴, —SO₂R²⁴, or —SO₂N(R²³)₂;
each R²³ is independently selected from hydrogen, C₁-C₆ alkyl, C₃-C₆ cycloalkyl, 3- to 6-membered heterocycloalkyl, phenyl, benzyl, 5-membered heteroaryl and 6-membered heteroaryl; or two R²³ groups are taken together with the N atom to which they are attached to form a 3- to 6-membered N-containing heterocycloalkyl;
each R²⁴ is independently selected from C₁-C₆ alkyl, C₃-C₆ cycloalkyl, 3- to 6-membered heterocycloalkyl, phenyl, benzyl, 5-membered heteroaryl and 6-membered heteroaryl.

55. The compound of claim 54, or a pharmaceutically acceptable salt thereof, wherein:
R³ is C₁-C₆ alkyl; and
R⁴ is C₁-C₆ alkyl;
or R³ and R⁴ are taken together with the carbon atom to which they are attached to form a C₃-C₆ cycloalkyl.

56. The compound of claim 54 or claim 55, or a pharmaceutically acceptable salt thereof, wherein:
R^{A} is C₁-C₄ alkyl, C₁-C₄ fluoroalkyl, C₃-C₆ cycloalkyl, 3- to 6-membered heterocycloalkyl, halogen, —CN, —SR^{A1}, —S(═O)R^{A1}, —S(═O)₂R^{A1}—OR^{A1}, —SF₅, or —SiMe₃;
R^{A1} is C₁-C₆ alkyl, C₁-C₆ fluoroalkyl, C₃-C₆ cycloalkyl, or 3- to 6-membered heterocycloalkyl; and
each R⁵ is independently hydrogen, halogen, C₁-C₆ alkyl, C₁-C₆ fluoroalkyl, C₃-C₆ cycloalkyl, 3- to 6-membered heterocycloalkyl, —CN, or —OR²¹.

57. The compound of any one of claims 54-56, or a pharmaceutically acceptable salt thereof, wherein:
R^{A} is C₁-C₄ alkyl, C₁-C₄ fluoroalkyl, or C₃-C₆ cycloalkyl; and
each R⁵ is independently hydrogen or C₁-C₆ alkyl.

58. The compound of any one of claims 54-57, or a pharmaceutically acceptable salt thereof, wherein:
X² is CR¹⁷; and
each R¹⁷ is hydrogen.

59. The compound of any one of claims 54-58, or a pharmaceutically acceptable salt thereof, wherein:
R¹⁸ is hydrogen, unsubstituted or substituted C₁-C₆ alkyl, unsubstituted or substituted C₁-C₆ fluoroalkyl, unsubstituted or substituted C₁-C₆ heteroalkyl, unsubstituted or substituted C₁-C₆ alkenyl, unsubstituted or substituted C₁-C₆ alkynyl;
or R¹⁸ is substituted C₁-C₆ alkyl, unsubstituted or substituted C₁-C₆ fluoroalkyl, unsubstituted or substituted C₁-C₆ heteroalkyl;
or R¹⁸ is C₁-C₆ alkyl which is substituted by —CN, —OH, or —OMe;
or R¹⁸ is unsubstituted or substituted C₃-C₁₀ cycloalkyl, unsubstituted or substituted 3- to 10-membered heterocycloalkyl, unsubstituted or substituted aryl, or unsubstituted or substituted heteroaryl;
or R¹⁸ is unsubstituted or substituted C₃-C₆ monocyclic cycloalkyl, unsubstituted or substituted C₅-C₁₀ bicyclic cycloalkyl, or unsubstituted or substituted 3- to 10-membered monocyclic or bicyclic heterocycloalkyl;
or R¹⁸ is unsubstituted or substituted 3- to 10-membered monocyclic or bicyclic heterocycloalkyl containing an oxygen atom;
or R¹⁸ is unsubstituted or substituted 5- to 10-membered bicyclic heterocycloalkyl which is a bridged, fused, or spirocyclic heterocycloalkyl;
or R¹⁸ is —CHR²⁰-(unsubstituted or substituted C₃-C₆ cycloalkyl), —CHR²⁰-(unsubstituted or substituted 3- to 6-membered heterocycloalkyl), —CHR²⁰-(unsubstituted or substituted phenyl), or —CHR²⁰-(unsubstituted or substituted 5- or 6-membered heteroaryl); and R²⁰ is hydrogen or —CH₃;
or R¹⁸ is —C(═O)R¹⁹, —CO₂R¹⁹, or —C(═O)NHR¹⁹; and R¹⁹ is unsubstituted or substituted C₁-C₆ alkyl, unsubstituted or substituted C₃-C₁₀ cycloalkyl, unsubstituted or substituted 3- to 10-membered heterocycloalkyl.

60. A compound of Formula (VI): 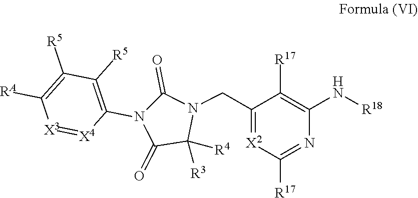
or a pharmaceutically acceptable salt thereof, wherein:
R^{A} is unsubstituted or substituted C₁-C₆ alkyl;
X³ and X⁴ are each independently CR or N;
R³ is hydrogen, unsubstituted or substituted C₁-C₆ alkyl, unsubstituted or substituted C₁-C₆ fluoroalkyl, unsubstituted or substituted C₁-C₆ alkoxy, unsubstituted or substituted phenyl, unsubstituted or substituted benzyl, unsubstituted or substituted monocyclic heteroaryl, unsubstituted or substituted C₃-C₆ cycloalkyl, or unsubstituted or substituted 3- to 6-membered heterocycloalkyl;
R⁴ is hydrogen or C₁-C₆ alkyl;
or R³ and R⁴ are taken together with the carbon atom to which they are attached to form a C₃-C₆ cycloalkyl or 3- to 6-membered heterocycloalkyl;
each R⁵ is independently hydrogen, halogen, unsubstituted or substituted C₁-C₆ alkyl, unsubstituted or substituted C₁-C₆ fluoroalkyl, —CN, —OH, —OR²¹, —CO₂R²², —N(R²²)₂, —C(═O)N(R²²)₂, —NR²²C(═O)R²¹, —S(═O)₂N(R²²)₂, or —NR²²S(═O)₂R²¹;
X² is CR¹⁷ or N;
each R¹⁷ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²¹, —CO₂R²², —N(R²²)₂, —C(═O)N(R²²)₂, —NR²²C(═O)R²¹, —S(═O)₂N(R²²)₂, or —NR²²S(═O)₂R²¹;
R¹⁸ is unsubstituted or substituted C₁-C₆ alkyl, unsubstituted or substituted C₁-C₆ fluoroalkyl, unsubstituted or substituted C₁-C₆ heteroalkyl, unsubstituted or substituted C₁-C₆ alkenyl, or unsubstituted or substituted C₁-C₆ alkynyl;
or R¹⁸ is substituted cyclopropyl, unsubstituted or substituted cyclobutyl, unsubstituted or substituted cyclopentyl, or unsubstituted or substituted cyclohexyl;
or R¹⁸ is unsubstituted or substituted C₅-C₁₀ bicyclic cycloalkyl;
or R¹⁸ is unsubstituted or substituted 3- to 10-membered monocyclic or bicyclic heterocycloalkyl containing an oxygen atom;
or R¹⁸ is —CHR²⁰-(unsubstituted or substituted carbocycle) or —CHR²⁰-(unsubstituted or substituted heterocycle);
R²⁰ is hydrogen or —CH₃;
each R²¹ is independently unsubstituted or substituted C₁-C₆ alkyl, unsubstituted or substituted C₃-C₆ cycloalkyl, unsubstituted or substituted 3- to 6-membered heterocycloalkyl, unsubstituted or substituted aryl, or unsubstituted or substituted heteroaryl;
each R²² is independently hydrogen, unsubstituted or substituted C₁-C₆ alkyl, unsubstituted or substituted C₃-C₆ cycloalkyl, unsubstituted or substituted 3- to 6-membered heterocycloalkyl, unsubstituted or substituted aryl, or unsubstituted or substituted heteroaryl; or two R²² on the same N atom are taken together with the N atom to which they are attached to form an unsubstituted or substituted N-containing heterocycloalkyl;
wherein each substituted alkyl, substituted fluoroalkyl, substituted aryl, substituted heteroaryl, substituted cycloalkyl, and substituted heterocycloalkyl is substituted with one or more R^{s} groups independently selected from the group consisting of halogen, C₁-C₆ alkyl, C₁-C₆ fluoroalkyl, C₃-C₆ cycloalkyl, 3- to 6-membered heterocycloalkyl, phenyl, benzyl, 5-membered heteroaryl, 6-membered heteroaryl, —CN, —OR²³, —CO₂R²³, —C(═O)N(R²³)₂, —N(R²³)₂, —NR²³C(═O)R²⁴, —SR²³—S(═O)R²⁴, —SO₂R²⁴, or —SO₂N(R²³)₂;
each R²³ is independently selected from hydrogen, C₁-C₆ alkyl, C₃-C₆ cycloalkyl, 3- to 6-membered heterocycloalkyl, phenyl, benzyl, 5-membered heteroaryl and 6-membered heteroaryl; or two R²³ groups are taken together with the N atom to which they are attached to form a 3- to 6-membered N-containing heterocycloalkyl;
each R²⁴ is independently selected from C₁-C₆ alkyl, C₃-C₆ cycloalkyl, 3- to 6-membered heterocycloalkyl, phenyl, benzyl, 5-membered heteroaryl and 6-membered heteroaryl.

61. The compound of claim 60, or a pharmaceutically acceptable salt thereof, wherein:
R³ is C₁-C₆ alkyl; and
R⁴ is C₁-C₆ alkyl;
or R³ and R⁴ are taken together with the carbon atom to which they are attached to form a C₃-C₆ cycloalkyl.

62. The compound of claim 60 or claim 61, or a pharmaceutically acceptable salt thereof, wherein:
each R⁵ is independently hydrogen, halogen, C₁-C₆ alkyl, C₁-C₆ fluoroalkyl, C₃-C₆ cycloalkyl, 3- to 6-membered heterocycloalkyl, —CN, or —OR²¹.

63. The compound of any one of claims 60-62, or a pharmaceutically acceptable salt thereof, wherein:
X³ and X⁴ are each CR⁵; and
each R⁵ is hydrogen.

64. The compound of any one of claims 60-63, or a pharmaceutically acceptable salt thereof, wherein:
R^{A} is C₁-C₄ alkyl.

65. The compound of any one of claims 60-64, or a pharmaceutically acceptable salt thereof, wherein:
R^{A} is isopropyl or tert-butyl.

66. The compound of any one of claims 60-65, or a pharmaceutically acceptable salt thereof, wherein:
X² is CR¹⁷; and
each R¹⁷ is independently hydrogen, halogen, C₁-C₆ alkyl, or C₁-C₆ fluoroalkyl.

67. The compound of any one of claims 60-66, or a pharmaceutically acceptable salt thereof, wherein:
X² is CR¹⁷; and
each R¹⁷ is hydrogen.

68. The compound of any one of claims 60-67, or a pharmaceutically acceptable salt thereof, wherein:
R⁸ is unsubstituted or substituted C₁-C₆ alkyl, unsubstituted or substituted C₁-C₆ fluoroalkyl, unsubstituted or substituted C₁-C₆ heteroalkyl, unsubstituted or substituted C₁-C₆ alkenyl, unsubstituted or substituted C₁-C₆ alkynyl.

69. The compound of claim 68, or a pharmaceutically acceptable salt thereof, wherein:
R¹⁸ is substituted C₁-C₆ alkyl, unsubstituted or substituted C₁-C₆ fluoroalkyl, unsubstituted or substituted C₁-C₆ heteroalkyl;
or R¹⁸ is C₁-C₆ alkyl which is substituted by —CN, —OH, or —OMe.

70. The compound of any one of claims 60-67, or a pharmaceutically acceptable salt thereof, wherein:
R¹⁸ is substituted cyclopropyl, unsubstituted or substituted cyclobutyl, unsubstituted or substituted cyclopentyl, or unsubstituted or substituted cyclohexyl.

71. The compound of any one of claims 60-67, or a pharmaceutically acceptable salt thereof, wherein:
R¹⁸ is unsubstituted or substituted C₅-C₁₀ bicyclic cycloalkyl which is a bridged, fused, or spirocyclic cycloalkyl.

72. The compound of any one of claims 60-67, or a pharmaceutically acceptable salt thereof, wherein:
R¹⁸ is unsubstituted or substituted 3- to 10-membered monocyclic or bicyclic heterocycloalkyl containing an oxygen atom;
or R¹⁸ is unsubstituted or substituted 5- to 10-membered bicyclic heterocycloalkyl containing an oxygen atom which is a bridged, fused, or spirocyclic heterocycloalkyl.

73. The compound of any one of claims 60-67, or a pharmaceutically acceptable salt thereof, wherein:
R¹⁸ is —CHR²⁰-(unsubstituted or substituted C₃-C₆ cycloalkyl), —CHR²⁰-(unsubstituted or substituted 3- to 6-membered heterocycloalkyl), —CHR²⁰-(unsubstituted or substituted phenyl), or —CHR²⁰-(unsubstituted or substituted 5- or 6-membered heteroaryl); and
R²⁰ is hydrogen or —CH₃.

74. A compound of Formula (VII): 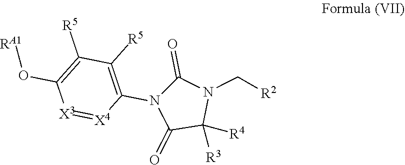
or a pharmaceutically acceptable salt thereof, wherein:
R^{A1} is C₁-C₆ alkyl, C₁-C₆ fluoroalkyl, C₃-C₆ cycloalkyl, or 3- to 6-membered heterocycloalkyl;
X³ and X¹ are each independently CR⁵ or N;
R² is 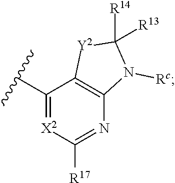
R^{c} is hydrogen, C₁-C₆ alkyl, C₁-C₆ fluoroalkyl, —C(═O)N(R²²)₂, —C(═O)R²¹, —C(═O)OR²², or —S(═O)₂R²¹;
X² is CR¹⁷ or N;
each R¹⁷ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²¹, —CO₂R²², —N(R²²)₂, —C(═O)N(R²²)₂, —OC(═O)R²¹, —NR²²C(═O)R²¹, —S(═O)₂N(R²²)₂, or —NR²²S(═O)₂R²¹,
R¹³ and R¹⁴ are each independently hydrogen, halogen, unsubstituted or substituted C₁-C₆ alkyl, C₁-C₆ fluoroalkyl, phenyl, monocyclic heteroaryl, C₃-C₆ cycloalkyl, 3- to 6-membered heterocycloalkyl, —CN, —OH, —OR^{2′}, —CO₂R²², —N(R²²)₂, —C(═O)N(R²)₂, or —OC(═O)R²¹;
or R¹³ and R¹⁴ are taken together with the carbon atom to which they are attached to form a —C(═O)—;
or R¹³ and R¹⁴ are taken together with the carbon atom to which they are attached to form a C₃-C₆ cycloalkyl or 3- to 6-membered heterocycloalkyl;
Y² is —(CR¹⁵R¹⁶)ₙ—, —O—, or —O—CR¹⁵R¹⁶—;
each R¹⁵ and R¹⁶ is independently hydrogen, halogen, cyano, C₁-C₆ alkyl, C₁-C₆ fluoroalkyl, C₁-C₆ alkoxy, phenyl, monocyclic heteroaryl, C₃-C₆ cycloalkyl, or 3- to 6-membered heterocycloalkyl;
or R¹⁴ and one R¹⁶ on an adjacent carbon atom are taken together to form an alkene bond;
n is 1 or 2;
or R² is 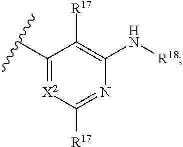
X² is CR¹⁷ or N;
each R¹⁷ is independently hydrogen, halogen, unsubstituted or substituted C₁-C₆ alkyl, unsubstituted or substituted C₁-C₆ fluoroalkyl, unsubstituted or substituted phenyl, unsubstituted or substituted monocyclic heteroaryl, unsubstituted or substituted C₃-C₆ cycloalkyl, unsubstituted or substituted 3- to 6-membered heterocycloalkyl, —CN, —OH, —OR²¹, —CO₂R²², —N(R²²)₂, —C(═O)N(R²²)₂, —NR²²C(═O)R^{2′}, —S(═O)₂N(R²²)₂, or —NR²²S(═O)₂R²¹;
R¹⁸ is substituted C₁-C₆ alkyl, unsubstituted or substituted C₁-C₆ fluoroalkyl, unsubstituted or substituted C₁-C₆ heteroalkyl, unsubstituted or substituted C₁-C₆ alkenyl, or unsubstituted or substituted C₁-C₆ alkynyl;
or R¹⁸ is unsubstituted or substituted cyclopropyl, unsubstituted or substituted cyclobutyl, unsubstituted or substituted cyclopentyl, or substituted cyclohexyl;
or R¹⁸ is unsubstituted or substituted C₅-C₁₀ bicyclic cycloalkyl;
or R¹⁸ is unsubstituted or substituted 3- to 10-membered monocyclic or bicyclic heterocycloalkyl;
or R¹⁸ is —CHR²⁰-(unsubstituted or substituted carbocycle) or —CHR²⁰-(unsubstituted or substituted heterocycle);
R²⁰ is hydrogen or —CH₃;
R³ is hydrogen, unsubstituted or substituted C₁-C₆ alkyl, unsubstituted or substituted C₁-C₆ fluoroalkyl, unsubstituted or substituted C₁-C₆ alkoxy, unsubstituted or substituted phenyl, unsubstituted or substituted benzyl, unsubstituted or substituted monocyclic heteroaryl, unsubstituted or substituted C₃-C₆ cycloalkyl, or unsubstituted or substituted 3- to 6-membered heterocycloalkyl;
R⁴ is hydrogen or C₁-C₆ alkyl;
or R³ and R⁴ are taken together with the carbon atom to which they are attached to form a C₃-C₆ cycloalkyl or 3- to 6-membered heterocycloalkyl;
each R⁵ is independently hydrogen, halogen, unsubstituted or substituted C₁-C₆ alkyl, unsubstituted or substituted C₁-C₆ fluoroalkyl, —CN, —OH, —OR²¹, —CO₂R²², —N(R²²)₂, —C(═O)N(R²²)₂, —NR²²C(═O)R²¹, —S(═O)₂N(R²²)₂, or —NR²²S(═O)₂R²¹;
each R²¹ is independently unsubstituted or substituted C₁-C₆ alkyl, unsubstituted or substituted C₃-C₆ cycloalkyl, unsubstituted or substituted 3- to 6-membered heterocycloalkyl, unsubstituted or substituted aryl, or unsubstituted or substituted heteroaryl;
each R²² is independently hydrogen, unsubstituted or substituted C₁-C₆ alkyl, unsubstituted or substituted C₃-C₆ cycloalkyl, unsubstituted or substituted 3- to 6-membered heterocycloalkyl, unsubstituted or substituted aryl, or unsubstituted or substituted heteroaryl; or two R²² on the same N atom are taken together with the N atom to which they are attached to form an unsubstituted or substituted N-containing heterocycloalkyl;
wherein each substituted alkyl, substituted fluoroalkyl, substituted aryl, substituted heteroaryl, substituted cycloalkyl, and substituted heterocycloalkyl is substituted with one or more R^{s} groups independently selected from the group consisting of halogen, C₁-C₆ alkyl, C₁-C₆ fluoroalkyl, C₃-C₆ cycloalkyl, 3- to 6-membered heterocycloalkyl, phenyl, benzyl, 5-membered heteroaryl, 6-membered heteroaryl, —CN, —OR², —CO₂R²³, —C(═O)N(R²³)₂, —N(R²³)₂, —NR²³C(═O)R²⁴, —SR²³—S(═O)R²⁴, —SO₂R²⁴, or —SO₂N(R²³)₂;
each R²³ is independently selected from hydrogen, C₁-C₆ alkyl, C₃-C₆ cycloalkyl, 3- to 6-membered heterocycloalkyl, phenyl, benzyl, 5-membered heteroaryl and 6-membered heteroaryl; or two R²³ groups are taken together with the N atom to which they are attached to form a 3- to 6-membered N-containing heterocycloalkyl;
each R²⁴ is independently selected from C₁-C₆ alkyl, C₃-C₆ cycloalkyl, 3- to 6-membered heterocycloalkyl, phenyl, benzyl, 5-membered heteroaryl and 6-membered heteroaryl.

75. The compound of claim 74, or a pharmaceutically acceptable salt thereof, wherein:
R³ is C₁-C₆ alkyl; and
R⁴ is C₁-C₆ alkyl;
or R³ and R⁴ are taken together with the carbon atom to which they are attached to form a C₃-C₆ cycloalkyl.

76. The compound of claim 74 or claim 75, or a pharmaceutically acceptable salt thereof, wherein:
each R⁵ is independently hydrogen, halogen, C₁-C₆ alkyl, C₁-C₆ fluoroalkyl, C₃-C₆ cycloalkyl, 3- to 6-membered heterocycloalkyl, —CN, or —OR²¹.

77. The compound of any one of claims 74-76, or a pharmaceutically acceptable salt thereof, wherein:
X³ and X⁴ are each CR⁵; and
each R⁵ is hydrogen.

78. The compound of any one of claims 74-77, or a pharmaceutically acceptable salt thereof, wherein:
R^{A1} is C₁-C₄ alkyl or C₁-C₄ fluoroalkyl.

79. The compound of any one of claims 74-78, or a pharmaceutically acceptable salt thereof, wherein:
R² is

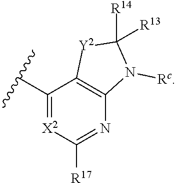

80. The compound of claim 79, or a pharmaceutically acceptable salt thereof, wherein:
R^{c} is hydrogen, C₁-C₆ alkyl, or —C(═O)OR²²;
X² is CR¹⁷;
each R¹⁷ is independently hydrogen, halogen, C₁-C₆ alkyl, or C₁-C₆ fluoroalkyl;
R¹³ and R¹⁴ are each independently hydrogen, halogen, C₁-C₆ alkyl, C₁-C₆ fluoroalkyl, C₃-C₆ cycloalkyl, or 3- to 6-membered heterocycloalkyl;
or R¹³ and R¹⁴ are taken together with the carbon atom to which they are attached to form a —C(═O)—;
or R¹³ and R¹⁴ are taken together with the carbon atom to which they are attached to form a C₃-C₄ cycloalkyl;
Y² is —(CR¹⁵R¹⁶)ₙ—, —O—, or —O—CR¹⁵R¹⁶—; and
each R¹⁵ and R¹⁶ is independently hydrogen, halogen, C₁-C₆ alkyl, C₁-C₆ fluoroalkyl, or C₃-C₆ cycloalkyl;
or R¹⁴ and one R¹⁶ on an adjacent carbon atom are taken together to form an alkene bond.

81. The compound of claim 79 or claim 80, or a pharmaceutically acceptable salt thereof, wherein:
R^{c} is hydrogen;
X² is CR¹⁷;
each R¹⁷ is hydrogen;
R¹³ and R⁴ are each hydrogen;
or R³ and R¹⁴ are taken together with the carbon atom to which they are attached to form a —C(═O)—; and
Y² is —(CR¹⁵R¹⁶)ₙ—, —O—, or —O—CR¹⁵R¹⁶—.

82. The compound of any one of claims 79-81, or a pharmaceutically acceptable salt thereof, wherein:
each R¹⁵ and R¹⁶ is independently hydrogen, C₁-C₆ alkyl, C₁-C₆ fluoroalkyl, or C₃-C₆ cycloalkyl.

83. The compound of any one of claims 79-82, or a pharmaceutically acceptable salt thereof, wherein: 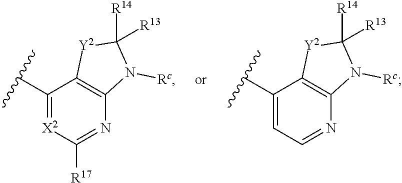
and
Y² is —CR¹⁵R¹⁶— or —CR¹⁵R¹⁶—CR¹⁵R¹⁶—.

84. The compound of any one of claims 79-83, or a pharmaceutically acceptable salt thereof, wherein:

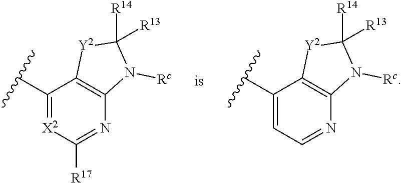

85. The compound of any one of claims 79-83, or a pharmaceutically acceptable salt thereof, wherein:
R¹⁴ and one R¹⁶ on an adjacent carbon atom are taken together to form an alkene bond.

86. The compound of any one of claims 74-78, or a pharmaceutically acceptable salt thereof, wherein:
R² is

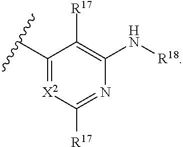

87. The compound of claim 86, or a pharmaceutically acceptable salt thereof, wherein:
X² is CR¹⁷; and
each R¹⁷ is independently hydrogen, halogen, C₁-C₆ alkyl, or C₁-C₆ fluoroalkyl.

88. The compound of claim 86 or claim 87, or a pharmaceutically acceptable salt thereof, wherein:
X² is CR¹⁷; and
each R¹⁷ is hydrogen.

89. The compound of any one of claims 86-88, or a pharmaceutically acceptable salt thereof, wherein:
R¹⁸ is substituted C₁-C₆ alkyl, unsubstituted or substituted C₁-C₆ fluoroalkyl, unsubstituted or substituted C₁-C₆ heteroalkyl, unsubstituted or substituted C₁-C₆ alkenyl, unsubstituted or substituted C₁-C₆ alkynyl.

90. The compound of claim 89, or a pharmaceutically acceptable salt thereof, wherein:
R¹⁸ is substituted C₁-C₆ alkyl, unsubstituted or substituted C₁-C₆ fluoroalkyl, unsubstituted or substituted C₁-C₆ heteroalkyl.
or R¹⁸ is C₁-C₆ alkyl which is substituted by —CN, —OH, or —OMe.

91. The compound of any one of claims 86-88, or a pharmaceutically acceptable salt thereof, wherein:
R¹⁸ is unsubstituted or substituted cyclopropyl, unsubstituted or substituted cyclobutyl, unsubstituted or substituted cyclopentyl, or substituted cyclohexyl.

92. The compound of any one of claims 86-88, or a pharmaceutically acceptable salt thereof, wherein:
R¹⁸ is unsubstituted or substituted C₅-C₁₀ bicyclic cycloalkyl which is a bridged, fused, or spirocyclic heterocycloalkyl.

93. The compound of any one of claims 86-88, or a pharmaceutically acceptable salt thereof, wherein:
R¹⁸ is unsubstituted or substituted 3- to 10-membered monocyclic or bicyclic heterocycloalkyl.

94. The compound of claim 93, or a pharmaceutically acceptable salt thereof, wherein:
R¹⁸ is unsubstituted or substituted 3- to 10-membered monocyclic or bicyclic heterocycloalkyl containing an oxygen atom;
or R¹⁸ is unsubstituted or substituted 5- to 10-membered bicyclic heterocycloalkyl which is a bridged, fused, or spirocyclic heterocycloalkyl.

95. The compound of any one of claims 86-88, or a pharmaceutically acceptable salt thereof, wherein:
R¹⁸ is —CHR²⁰-(unsubstituted or substituted C₃-C₆ cycloalkyl), —CHR²⁰-(unsubstituted or substituted 3- to 6-membered heterocycloalkyl), —CHR²⁰-(unsubstituted or substituted phenyl), or —CHR²⁰-(unsubstituted or substituted 5- or 6-membered heteroaryl); and
R²⁰ is hydrogen or —CH₃.

96. A compound of Formula (VIII): 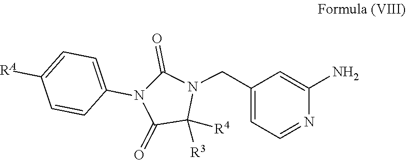
or a pharmaceutically acceptable salt thereof, wherein:
R^{A} is —SCF₃, —S(═O)CF₃, or —S(═O)₂CF₃;
R³ is C₂-C₆ alkyl which is unsubstituted or substituted by —OH, phenyl, benzyl, C₃-C₆ cycloalkyl, or —CH₂OH; and
R⁴ is hydrogen or methyl;
or R³ and R⁴ are taken together with the carbon atom to which they are attached to form a cyclobutyl, cyclopentyl, or cyclohexyl.

97. The compound of claim 96, or a pharmaceutically acceptable salt thereof, wherein:
R^{A} is —SCF₃ or —S(═O)₂CF₃.

98. The compound of claim 96 or claim 97, or a pharmaceutically acceptable salt thereof, wherein:
R³ is C₂-C₆ alkyl which is unsubstituted or substituted by —OH, phenyl, benzyl, C₃-C₆ cycloalkyl, or —CH₂OH; and
R⁴ is hydrogen or methyl.

99. The compound of claim 98, or a pharmaceutically acceptable salt thereof, wherein:
R³ is ethyl, propyl, isopropyl, butyl, isobutyl, sec-butyl, phenyl, benzyl, cyclopropyl, cyclobutyl, cyclopentyl, cyclohexyl, or —CH₂OH; and
R⁴ is hydrogen or methyl.

100. The compound of claim 96 or claim 97, or a pharmaceutically acceptable salt thereof, wherein:
R³ and R⁴ are taken together with the carbon atom to which they are attached to form a cyclobutyl, cyclopentyl, or cyclohexyl.

101. A compound of Formula (IX): 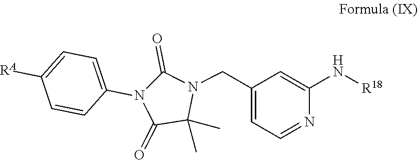
or a pharmaceutically acceptable salt thereof, wherein:
R^{A} is —SR^{A1}, —S(═O)R^{A1}, or —S(═O)₂R^{A1};
R^{A1} is C₁-C₄ alkyl, C₁-C₄ fluoroalkyl, or C₃-C₆ cycloalkyl;
R¹⁸ is C₁-C₆ alkyl which is substituted by —CN, —OH, or —OMe;
or R¹⁸ is unsubstituted or substituted C₁-C₆ fluoroalkyl or unsubstituted or substituted C₁-C₆ heteroalkyl;
or R¹⁸ is unsubstituted or substituted 3- to 10-membered monocyclic or bicyclic heterocycloalkyl;
or R¹⁸ is —CHR²⁰-(unsubstituted or substituted C₃-C₆ cycloalkyl), —CHR²⁰-(unsubstituted or substituted 3- to 6-membered heterocycloalkyl), or —CHR²⁰-(unsubstituted or substituted 5-membered heteroaryl);
R²⁰ is hydrogen or —CH₃;
or R¹⁸ is —C(═O)R¹⁹, where R¹⁹ is unsubstituted or substituted 3- to 10-membered heterocycloalkyl;
or R¹⁸ is —C(═O)NHR¹⁹, where R¹⁹ is unsubstituted or substituted C₃-C₁₀ cycloalkyl or unsubstituted or substituted 3- to 10-membered heterocycloalkyl;
wherein each substituted alkyl, substituted fluoroalkyl, substituted aryl, substituted heteroaryl, substituted cycloalkyl, and substituted heterocycloalkyl is substituted with one or more R^{s} groups independently selected from the group consisting of halogen, C₁-C₆ alkyl, C₁-C₆ fluoroalkyl, C₃-C₆ cycloalkyl, 3- to 6-membered heterocycloalkyl, phenyl, benzyl, 5-membered heteroaryl, 6-membered heteroaryl, —CN, —OR²³, —CO₂R³, —C(═O)N(R²³)₂, —N(R²³)₂, —NR²³C(═O)R²⁴, —SR²³—S(═O)R²⁴, —SO₂R²⁴, or —SO₂N(R²³)₂;
each R²³ is independently selected from hydrogen, C₁-C₆ alkyl, C₃-C₆ cycloalkyl, 3- to 6-membered heterocycloalkyl, phenyl, benzyl, 5-membered heteroaryl and 6-membered heteroaryl; or two R³ groups are taken together with the N atom to which they are attached to form a 3- to 6-membered N-containing heterocycloalkyl;
each R²⁴ is independently selected from C₁-C₆ alkyl, C₃-C₆ cycloalkyl, 3- to 6-membered heterocycloalkyl, phenyl, benzyl, 5-membered heteroaryl and 6-membered heteroaryl.

102. The compound of claim 101, or a pharmaceutically acceptable salt thereof, wherein:
R^{A1} is C₁-C₄ alkyl or C₁-C₄ fluoroalkyl.

103. The compound of claim 101 or claim 102, or a pharmaceutically acceptable salt thereof, wherein:
R^{A} is —SCF₃, —S(═O)CF₃, —S(═O)₂CF₃, —S(═O)₂CHF₂, —S(═O)₂C(CH₃)₃, or —S(═O)₂-(cyclopropyl).

104. The compound of any one of claims 101-103, or a pharmaceutically acceptable salt thereof, wherein:
R¹⁸ is C₁-C₆ alkyl which is substituted by —CN, —OH, or —OMe.

105. The compound of any one of claims 101-103, or a pharmaceutically acceptable salt thereof, wherein:
R¹⁸ is unsubstituted or substituted C₁-C₆ fluoroalkyl or unsubstituted or substituted C₁-C₆ heteroalkyl.

106. The compound of any one of claims 101-103, or a pharmaceutically acceptable salt thereof, wherein:
or R¹⁸ is unsubstituted or substituted 3- to 10-membered monocyclic or bicyclic heterocycloalkyl.

107. The compound of claim 106, or a pharmaceutically acceptable salt thereof, wherein:
R¹⁸ is unsubstituted or substituted 3- to 10-membered monocyclic or bicyclic heterocycloalkyl containing an oxygen atom;
or R¹⁸ is unsubstituted or substituted 5- to 10-membered bicyclic heterocycloalkyl which is a bridged, fused, or spirocyclic heterocycloalkyl.

108. The compound of any one of claims 101-103, or a pharmaceutically acceptable salt thereof, wherein:
or R¹⁸ is —CHR²⁰-(unsubstituted or substituted C₃-C₆ cycloalkyl), —CHR²⁰-(unsubstituted or substituted 3- to 6-membered heterocycloalkyl), or —CHR²¹-(unsubstituted or substituted 5-membered heteroaryl); and R²⁰ is hydrogen or —CH₃.

109. The compound of any one of claims 101-103, or a pharmaceutically acceptable salt thereof, wherein:
R¹⁸ is —C(═O)R¹⁹, where R¹⁹ is unsubstituted or substituted 3- to 10-membered heterocycloalkyl.

110. The compound of any one of claims 101-103, or a pharmaceutically acceptable salt thereof, wherein:
R¹⁸ is —C(═O)NHR¹⁹, where R⁹ is unsubstituted or substituted C₃-C₁₀ cycloalkyl or unsubstituted or substituted 3- to 10-membered heterocycloalkyl.

111. A compound selected from any one of Table 1, Table 2, Table 3, Table 4, Table 5, Table 6, Table 7, Table 8, Table 9, or Table 10, or a pharmaceutically acceptable salt thereof.

112. A pharmaceutical composition comprising a compound of any one of claims 1-111, or a pharmaceutically acceptable salt thereof, and at least one pharmaceutically acceptable excipient.

113. The pharmaceutical composition of claim 112, wherein the pharmaceutical composition is formulated for administration to a mammal by intravenous administration, subcutaneous administration, oral administration, inhalation, nasal administration, dermal administration, or ophthalmic administration.

114. The pharmaceutical composition of claim 112, wherein the pharmaceutical composition is in the form of a tablet, a pill, a capsule, a liquid, a suspension, a gel, a dispersion, a solution, an emulsion, an ointment, or a lotion.

115. Use of a compound of any one of claims 1-111, or a pharmaceutically acceptable salt hereof, in the treatment of a disease or condition in a mammal that would benefit from the modulation of insulin-like growth factor-1 receptor (IGF-1R).

116. The use of claim 115, wherein the disease or condition is thyroid eye disease (TED).
